(19) United States
(12) Patent Application Publication
(10) Pub. No.: US 2023/0265098 A1
(43) Pub. Date: Aug. 24, 2023

(54) ALKYNE QUINAZOLINE DERIVATIVES AS INHIBITORS OF ERBB2

(71) Applicant: EnLiven Therapeutics, Inc., Boulder, CO (US)

(72) Inventor: Joseph P. LYSSIKATOS, Boulder, CO (US)

(21) Appl. No.: 18/012,488

(22) PCT Filed: Jul. 1, 2021
(86) PCT No.: PCT/US2021/040071

§ 371 Date: Dec. 22, 2022

Related U.S. Application Data
(60) Provisional application No. 63/160,606, filed on Mar. 12, 2021.
(60) Provisional application No. 63/047,776, filed on Jul. 2, 2020.

(51) Int. Cl. C07D 487/04; C07D 471/04; C07D 401/12; C07D 403/12; A61P 35/00

(52) U.S. Cl. CPC C07D 487/04; C07D 471/04; C07D 401/12; C07D 403/12; A61P 35/00

(57) ABSTRACT

The present disclosure relates generally to compounds and compositions thereof for inhibition of ErbB2, including mutant forms of ErbB2, particularly those harboring an Exon 20 mutation, methods of preparing said compounds and compositions, and their use in the treatment or prophylaxis of various cancers, such as lung, glioma, skin, head neck, salivary gland, breast, esophageal, liver, stomach (gastric), uterine, cervical, biliary tract, pancreatic, colorectal, renal, bladder or prostate cancer.

### CROSS-REFERENCE TO RELATED APPLICATIONS

This application claims priority to and the benefit of U.S. Provisional Application No. 63/047,776, filed on Jul. 2, 2020, and U.S. Provisional Application No. 63/160,606, filed on Mar. 12, 2021, the disclosures of each of which are incorporated herein by reference in their entireties.

### FIELD OF THE INVENTION

The present disclosure relates generally to compounds and compositions thereof for inhibition of ErbB2, including mutant forms of ErbB2, particularly those harboring an Exon 20 mutation, methods of preparing said compounds and compositions, and their use in the treatment or prophylaxis of various cancers, such as lung, glioma, skin, head and neck, salivary gland, breast, esophageal, liver, stomach (gastric), uterine, cervical, biliary tract, pancreatic, colorectal, renal, bladder or prostate cancer.

### BACKGROUND

ErbB2 (or HER2) is a member of the ErbB receptor tyrosine kinase family consisting of four related receptors, including ErbB1 (also known as epidermal growth factor receptor, or EGFR), ErbB3 and ErbB4. Although there are no known ligands that bind to monomeric ErbB2, it can dimerize with other ErbB receptors, particularly ErbB3, and regulate downstream signaling cascades including, but not limited to, the MAPK and PI3K pathways, that promote cell proliferation and survival. Aberrant overexpression of ErbB2 or certain genetic alterations (including point mutations that lead to certain amino acid substitutions or small in-frame insertions in Exon 20 that lead to the deletion and/or insertion of certain small stretches of amino acids) are known to confer elevated or constitutive tyrosine kinase activation to the receptor. Accordingly, the overexpression or mutation of ErbB2 is highly associated with aggressive forms of solid cancers, including breast, ovarian, stomach, and lung cancer (NSCLC).

Currently, there are few approved treatments for cancers associated with ErbB2 overexpression, including tyrosine kinase inhibitors (TKIs) such as tucatinib. Although these TKIs can be effective at ameliorating cancers associated with ErbB2 overexpression, their therapeutic utility is often limited by inadequate selectivity for ErbB2 over EGFR, and consequently are dose-limited by toxicity concerns related to EGFR inhibition (especially gastrointestinal and skin-related toxicities). These toxicities necessitate restrictive dosing regimens, leading to suboptimal target engagement and thus limited therapeutic benefit. Moreover, while current TKIs provide therapeutic benefit for cancers driven by ErbB2 overexpression, they may have limited efficacy in patients harboring specific genetic alterations, such as EGFR or ERBB2 exon 20 insertions, specific point mutations or genetic alterations associated with ErbB family ligands, such as NRG1 gene fusions.

For example, in a small proportion of lung cancer patients, certain especially pernicious mutations in EGFR and ErbB2 known as EGFR exon 20 insertions/ErbB2 insertions are markedly less sensitive to first and second generation reversible TKIs. An added challenge to the development of viable therapies for these specific ErbB Exon 20 mutants (20ins or E20I) is the fact these alterations are heterogeneous, encompassing a diversity of amino acid insertions/deletions. In addition to E20I mutations, a number of other genetic alterations of the receptor, specifically point mutations leading to single amino acid substitutions, have been associated with the development of a variety of cancers, including lung cancer. Although the resistance mechanisms associated with each of these mutations are not fully understood, it is believed that the mutations may share a commonality in promoting ligand-independent activation of the kinases. Further investigation of the underlying mechanisms and development of TKIs tailored to these mutants are needed.

Other aggressive, refractory cancers exhibiting ErbB2 overexpression have been observed to harbor NRG1 gene re-arrangements resulting in novel fusion proteins. NRG1 gene fusions may induce overproduction of neuregulin-1, the cognate ligand for ErbB3. The simultaneous overexpression of ErbB2 and overproduction of neuregulin-1 may lead to excess activation of ErbB2-ErbB3 heterodimers and resultant hyperplasia.

Accordingly, there remains a need for new therapeutics for the treatment of cancers driven by dysregulated ErbB2 receptor kinase activity, not only with improved safety and selectivity for ErbB2 over EGFR, but also for addressing mutation-associated sub-variants of ErbB2 (e.g., E20I mutations and NRG1 gene fusions) with enhanced potency.

### SUMMARY OF THE INVENTION

In one aspect, provided is a compound of formula (I)

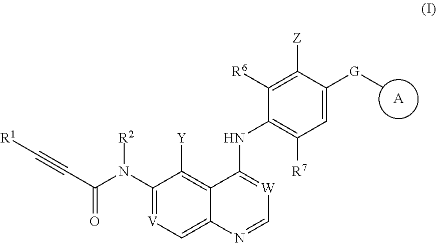

or a pharmaceutically acceptable salt thereof, wherein:
ring A is

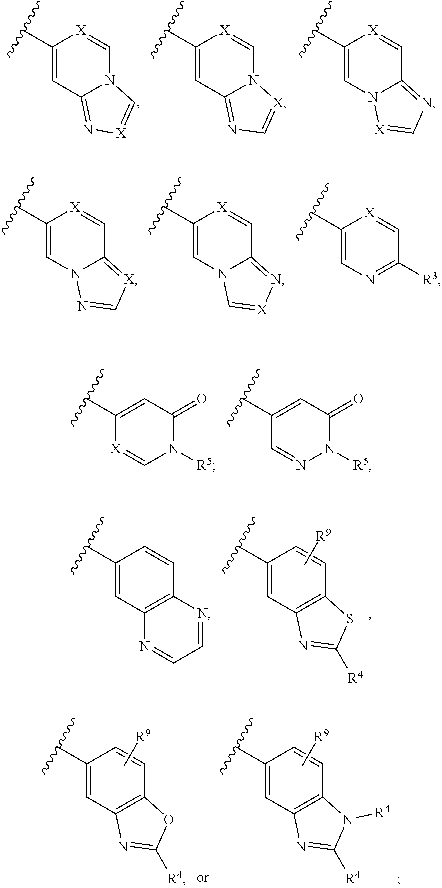

V is N or C—R⁸; W is N or C—CN; each X is independently N or CH; G is CH₂, CH(CH₃),

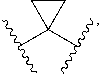 [SMILES: *C1(*)CC1]

O, or S; Y is H, F, or —O(C₁-C₃ alkyl); Z is H, F, Cl, C₁-C₂ alkyl, or cyclopropyl; R¹ is H, C₁-C₄ alkyl, C₃-C₆ cycloalkyl, or 4- to 6-membered heterocyclyl containing 1-2 ring heteroatoms selected from the group consisting of N, O and S, wherein the C₁-C₃ alkyl is optionally substituted by 1-3 substituents selected from the group consisting of F, —OH, —OPO₃²⁻, —N(C₁-C₃ alkyl)(C₁-C₃ alkyl), and 4- to 6-membered heterocyclyl containing 1-2 ring heteroatoms selected from the group consisting of N and O; and wherein the C₃-C₆ cycloalkyl and 4- to 6-membered heterocyclyl are optionally substituted by 1-3 substituents selected from the group consisting of F, —OH, C₁-C₃ alkyl, —N(C₁-C₃ alkyl)(C₁-C₃ alkyl), and 4- to 6-membered heterocyclyl containing 1-2 ring heteroatoms selected from the group consisting of N and O; R² is H, C₁-C₃ alkyl or cyclopropyl; R³ is H, —CD₃, C₁-C₃ alkyl, —CF₂H, —CF₃, —CFH—CFH₂, allyl, —CH₂-cyclopropyl, cyclopropyl, —CN, —SR⁴, —S(O)(C₁-C₃ alkyl), or —S(O)₂(C₁-C₃ alkyl); each R⁴ is independently H, C₁-C₃ alkyl, —CF₂H, —CF₃, or cyclopropyl; R⁵ is C₁-C₃ alkyl, —CD₃, —CF₂H, —CF₃, allyl, —CH₂-cyclopropyl, cyclopropyl, or —OR⁴; R⁶ is H or F; R⁷ is H or F; R⁸ is H or —O(C₁-C₃ alkyl), wherein the C₁-C₃ alkyl is optionally substituted by 1-4 substituents selected from the group consisting of —F, —OH, —OR^{8a} and —NR^{8a}R^{8b}, wherein each R^{8a} and R^{8b} are independently H or C₁-C₃ alkyl or wherein each pair of geminal R^{8a} and R^{8b} may be taken together with the nitrogen atom to which they are attached to form an N-heterocycloalkyl wherein the N-heterocycloalkyl is optionally substituted by C₁-C₃ alkyl; and R⁹ is H, halogen C₁-C₃ alkyl —CF₂H, —CF₃, cyclopropyl, —CN or —OR⁴.

In some embodiments, R⁸ is —O(C₂-C₃ alkyl), wherein the C₂-C₃ alkyl is substituted by 1-2 substituents selected from the group consisting of —OH and —NR^{8a}R^{8b}, wherein each R^{8a} and R^{8b} are independently H or C₁-C₃ alkyl. In other embodiments, R⁸ is —OCH₂CH₂OH, —OCH₂CH₂N(CH₃)₂, or —OCH₂CH₂CH₂N(CH₃)₂. In yet other embodiments, R⁸ is H. In some embodiments, which may be combined with any of the preceding embodiments, W is N. In other embodiments, W is C—CN.

In some embodiments of the present aspect, the compound of formula (I) is a compound of formula (II)

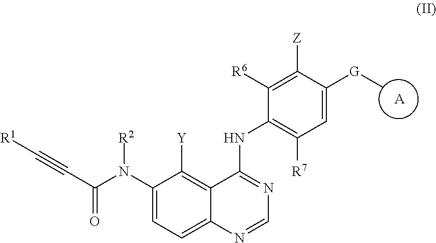

or a pharmaceutically acceptable salt thereof, wherein:
ring A is

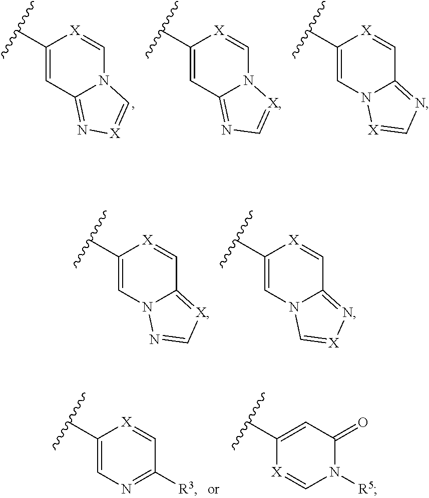

each X is independently N or CH; G is CH₂, O, or S; Y is H, F, or —O(C₁-C₃ alkyl); Z is H, F, C₁, C₁-C₂ alkyl, or cyclopropyl; R¹ is H, C₁-C₃ alkyl, C₃-C₆ cycloalkyl, or 4- to 6-membered heterocyclyl containing 1-2 ring heteroatoms selected from the group consisting of N, O and S, wherein the C₁-C₃ alkyl, C₃-C₆ cycloalkyl, and 4- to 6-membered heterocyclyl are optionally substituted by 1-3 substituents selected from the group consisting of F, —OH, —N(C₁-C₃ alkyl)(C₁-C₃ alkyl), and 4- to 6-membered heterocyclyl containing 1-2 ring heteroatoms selected from the group consisting of N and O; R² is H, C₁-C₃ alkyl or cyclopropyl; R³ is H, —CD₃, C₁-C₃ alkyl, —CF₂H, —CF₃, allyl, —CH₂-cyclopropyl, cyclopropyl, —CN, —SR⁴, —S(O)(C₁-C₃ alkyl), or —S(O)₂(C₁-C₃ alkyl); R⁴ is H, C₁-C₃ alkyl, —CF₂H, —CF₃, or cyclopropyl; R⁵ is C₁-C₃ alkyl, —CD₃, —CF₂H, —CF₃, allyl, —CH₂-cyclopropyl, cyclopropyl, or —OR⁴; R⁶ is H or F; and R⁷ is H or F.

In some embodiments of the present aspect, ring A is

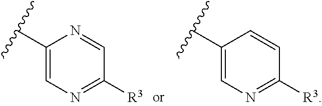

In yet another embodiment of this aspect, R³ is H, —CD₃, C₁-C₂ alkyl, CF₂H, CF₃, cyclopropyl, —CN, —SR⁴, —S(O)(C₁-C₂ alkyl), or —S(O)₂(C₁-C₂ alkyl). In still further embodiments, R³ is H, —CD₃, —CH₃, —CF₂H, —CF₃, cyclopropyl, —CN, —SR⁴, —S(O)(CH₃), or —S(O)₂(CH₃). In still yet another embodiment, R³ is —CH₃ or —CD₃. In still yet another embodiment, R³ is cyclopropyl. In still yet another embodiment, R³ is —CFH—CFH₂. In other embodiments of the present aspect, ring A is

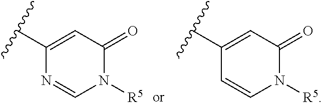

In further embodiments of the preceding embodiment, R⁵ is C₁-C₂ alkyl, —CD₃, CF₂H, CF₃, allyl, —CH₂-cyclopropyl, cyclopropyl, or —OR⁴. In still other embodiments, R⁵ is —CH₃ or —CD₃. In still other embodiments, R⁵ is —CH₂-cyclopropyl or cyclopropyl. In other embodiments of the present aspect, ring A is

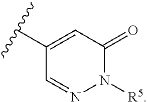

In further embodiments of the preceding embodiment, R⁵ is C₁-C₂ alkyl, —CD₃, CF₂H, CF₃, allyl, —CH₂-cyclopropyl, cyclopropyl, or —OR⁴. In still other embodiments, R⁵ is —CH₃ or —CD₃. In still other embodiments, R⁵ is —CH₂-cyclopropyl or cyclopropyl. In other embodiments of the present aspect, ring A is

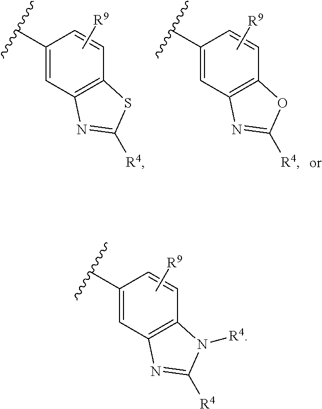

In further embodiments of the preceding embodiment, R⁹ is H, halogen, C₁-C₃ alkyl, —CF₂H, —CF₃, cyclopropyl, —CN or —OR⁴. In still other embodiments, R⁹ is —CH₃.

In yet other embodiments which may be combined with any of the preceding embodiments, each R⁴ is independently H, C₁-C₂ alkyl, —CF₂H, —CF₃, or cyclopropyl. In certain embodiments, each R⁴ is independently H, —CH₃, —CHF₂, or cyclopropyl.

In additional embodiments of the present aspect, ring A is

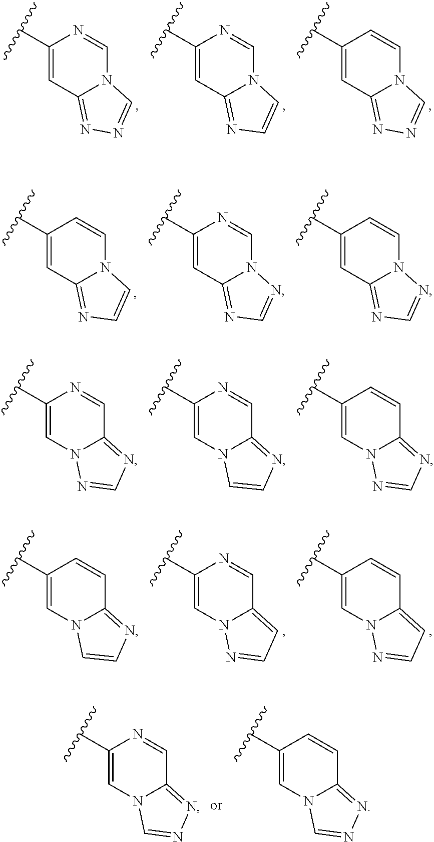

In still other embodiments of the present aspect, ring A is

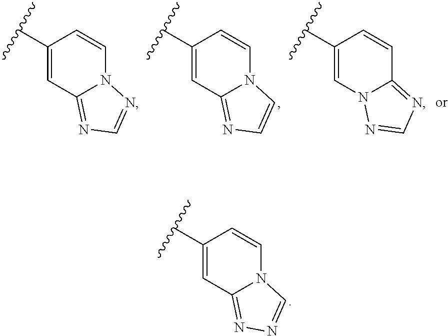 [SMILES: *c1ccn2ncnc2c1 ; *c1ccn2ccnc2c1 ; *c1ccc2ncnn2c1 ; *c1ccn2cnnc2c1]

In certain embodiments, ring A is

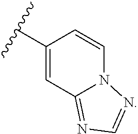 [SMILES: *c1ccn2ncnc2c1]

In still other embodiments, ring A is

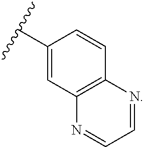 [SMILES: *c1ccc2nccnc2c1]

In some embodiments of the present aspect, which may be combined with any of the preceding embodiments, Z is H, F, Cl, or —CH₃. In certain embodiments, Z is —CH₃.

In still other embodiments, which may be combined with any of the preceding embodiments, R¹ is H, C₁-C₂ alkyl, cyclopropyl, or 4- to 6-membered heterocyclyl containing 1-2 ring heteroatoms selected from the group consisting of N, O and S, wherein the C₁-C₂ alkyl, cyclopropyl, and 4- to 6-membered heterocyclyl are optionally substituted by 1-3 substituents selected from the group consisting of F, —OH, —N(C₁-C₃ alkyl)(C₁-C₃ alkyl), and 4- to 6-membered heterocyclyl containing 1-2 ring heteroatoms selected from the group consisting of N and O. In some embodiments, R¹ is C₁-C₂ alkyl optionally substituted by —OH or —N(C₁-C₃ alkyl)(C₁-C₃ alkyl). In certain embodiments, R¹ is —CH₃, —CH₂OH, —(CH₂)₂OH or —CH₂N(CH₃)₂. In still other embodiments, R¹ is —CH₃ or —(CH₂)₂OH.

In yet further embodiments, which may be combined with any of the preceding embodiments, Y is is H, F, or —O(C₁-C₂ alkyl). In some embodiments, Y is H, F, or —OCH₃. In certain embodiments, Y is H. In still additional embodiments, which may be combined with any of the preceding embodiments, R² is H or C₁-C₃ alkyl. In some embodiments, R² is H, —CH₃, or —CH₂CH₃. In certain embodiments, R² is H. In some embodiments, which may be combined with any of the preceding embodiments, G is O. In other embodiments, G is CH₂. In other embodiments, G is CH(CH₃). In other embodiments, G is

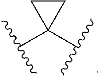 [SMILES: *C1(*)CC1]

In yet other embodiments, G is S. In other embodiments, R⁶ is H. In some embodiments, R⁶ is F. In still further embodiments, R⁷ is H. In yet other embodiments, R⁷ is F.

In another aspect, provided herein are compounds as described herein in Table 1. In yet another aspect, provided herein are pharmaceutical compositions comprising a compound of formula (I) or formula (II) as described herein, or a pharmaceutically acceptable salt thereof, and at least one pharmaceutically acceptable excipient.

In one aspect, provided herein is a method of inhibiting kinase activity of a human receptor tyrosine kinase ErbB2 or a mutant form of human ErbB2 comprising contacting the ErbB2 or the mutant form with a therapeutically effective amount of a compound of formula (I) or formula (II), or a pharmaceutically acceptable salt thereof, as described herein, or a therapeutically effective amount of the pharmaceutical composition as described herein. In some embodiments of the present aspect, the mutant form of human ErbB2 comprises a mutation in Exon 20. In further embodiments of the present aspect, the mutant form of human ErbB2 comprises one or more mutations that introduce amino acid deletions and/or insertions selected from the group consisting of: A775_A776insYVMA, G778_P780insGSP, G776delinsVC, P780_Y781insGSP, M774delinsWLV, A775_G776insSVMA, A775_G776insI, G776delinsLC, G778_S779InsCPG, and V777_G778insGSP. In other embodiments of the present aspect, the mutant form of human ErbB2 comprises a disease-associated point mutation in ErbB2. In still further embodiments of the present aspect, the mutant form of human ErbB2 comprises one or more point mutations in ErbB2 that introduce amino acid substitutions selected from the group consisting of P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, N1219S, and A1232fs. In still further embodiments of the present aspect, the mutant form of human ErbB2 comprises one or more point mutations in ErbB2 that introduce (a) an amino acid substitution selected from the group consisting of P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, and N1219S; or (b) a frameshift at A1232.

In yet another aspect, provided herein is a method of treating a patient having a cancer, comprising administering to the patient a therapeutically effective amount of a compound of formula (I) or formula (II), or a pharmaceutically acceptable salt thereof, as described herein, or a therapeutically effective amount of the pharmaceutical composition as described herein. In some embodiments of the present aspect, the cancer comprises cells or cell tissue having increased ErbB2 kinase activity. In some embodiments of the present aspect, the cancer comprises cells or cell tissue having increased ErbB2 kinase activity as compared to a control. In certain embodiments, the cancer comprises cells or cell tissue having increased ErbB2 kinase activity as compared to ErbB2 kinase activity in control cell or in control cell tissue. In some embodiments, the increased ErbB2 kinase activity is associated with a mutant form of human ErbB2. In further embodiments of the present aspect, the cancer comprises cells or cell tissue having one or more mutations in Exon 20 of the ErbB2. In certain embodiments, the cancer comprises cells or cell tissue having one or more mutations in Exon 20 of the ErbB2 that introduce amino acid deletions and/or insertions selected from the group consisting of A775_A776insYVMA, G778_P780insGSP, G776delinsVC, P780_Y781insGSP, M774delinsWLV, A775_G776insSVMA, A775_G776insI, G776delinsLC, G778_S779InsCPG, and V777_G778insGSP. In other embodiments, the cancer comprises cells or cell tissue having one or more disease-associated point mutations in ErbB2. In certain other embodiments, the cancer comprises cells or cell tissue having one or more point mutations that introduce amino acid substitutions selected from the group consisting of P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, N1219S, and A1232fs. In certain other embodiments, the cancer comprises cells or cell tissue having one or more point mutations that introduce (a) an amino acid substitution selected from the group consisting of P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, and N1219S; or (b) a frameshift at A1232.

In some embodiments of the present aspect, which may be combined with any of the preceding embodiments, the cancer is lung, glioma, skin, head and neck, salivary gland, breast, esophageal, liver, stomach (gastric), uterine, cervical, biliary tract, pancreatic, colorectal, renal, bladder or prostate cancer. In certain embodiments, the cancer is non-small cell lung cancer. In still other embodiments, which may be combined with any of the preceding embodiments, the patient has received at least one, at least two, or at least three prior therapies for the cancer. In certain embodiments, one or more of the prior therapies selected from the group consisting of lapatinib, neratinib, afatinib, pyrotinib, poziotinib, TAK-788, and tucatinib.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

### I. Definitions

As used herein, the following definitions shall apply unless otherwise indicated. Further, if any term or symbol used herein is not defined as set forth below, it shall have its ordinary meaning in the art.

The term “excipient” as used herein means an inert or inactive substance that may be used in the production of a drug or pharmaceutical, such as a tablet containing a compound of the present disclosure as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

The terms “individual”, “subject” and “patient” refer to mammals and includes humans and non-human mammals. Examples of patients include, but are not limited to, mice, rats, hamsters, guinea pigs, pigs, rabbits, cats, dogs, goats, sheep, cows, and humans. In some embodiments, patient refers to a human.

As used herein, the term “mammal” includes, but is not limited to, humans, mice, rats, guinea pigs, monkeys, dogs, cats, horses, cows, pigs, and sheep.

“Pharmaceutically acceptable” refers to safe and non-toxic, and suitable for in vivo or for human administration.

As used herein, the term “alkyl”, by itself or as part of another substituent, means, unless otherwise stated, a straight or branched chain hydrocarbon radical, having the number of carbon atoms designated (i.e., C₁-C₆ means one to six carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. In some embodiments, the term “alkyl” may encompass C₁-C₆ alkyl, C₂-C₆ alkyl, C₃-C₆ alkyl, C₄-C₆ alkyl, C₅-C₆ alkyl, C₁-C₅ alkyl, C₂-C₅ alkyl, C₃-C₅ alkyl, C₄-C₅ alkyl, C₁-C₄ alkyl, C₂-C₄ alkyl, C₃-C₄ alkyl, C₁-C₃ alkyl, C₂-C₃ alkyl, or C₁-C₂ alkyl.

The term “cycloalkyl” refers to hydrocarbon rings having the indicated number of ring atoms (e.g., C₃-C₆ cycloalkyl means 3-6 carbons) and being fully saturated or having no more than one double bond between ring vertices. In some embodiments, “cycloalkyl” encompasses C₃-C₇ cycloalkyl, C₄-C₇ cycloalkyl, C₅-C₇ cycloalkyl, C₅-C₇ cycloalkyl, C₃-C₆ cycloalkyl, C₄-C₆ cycloalkyl, C₅-C₆ cycloalkyl, C₃-C₅ cycloalkyl, C₄-C₅ cycloalkyl, or C₃-C₄ cycloalkyl.

The term “heterocycloalkyl”, “heterocyclic”, “heterocyclyl”, or “heterocycle” refers to a cycloalkyl radical group having the indicated number of ring atoms (e.g., 5-6 membered heterocycloalkyl) that contain from one to five heteroatoms selected from the group consisting of N, O, and S, wherein the nitrogen and sulfur atoms are optionally oxidized, nitrogen atom(s) are optionally quaternized, as ring atoms. Unless otherwise stated, a “heterocycloalkyl,” “heterocyclic,” or “heterocycle” ring can be a monocyclic, a bicyclic, spirocyclic or a polycylic ring system. Non-limiting examples of “heterocycloalkyl,” “heterocyclic,” or “heterocycle” rings include pyrrolidine, piperidine, N-methylpiperidine, imidazolidine, pyrazolidine, butyrolactam, valerolactam, imidazolidinone, hydantoin, dioxolane, phthalimide, piperidine, pyrimidine-2,4(1H,3H)-dione, 1,4-dioxane, morpholine, thiomorpholine, thiomorpholine-5-oxide, thiomorpholine-S,S-oxide, piperazine, pyran, pyridone, 3-pyrroline, thiopyran, pyrone, tetrahydrofuran, tetrahydrothiophene, quinuclidine, tropane and the like. A “heterocycloalkyl,” “heterocyclic,” or “heterocycle” group can be attached to the remainder of the molecule through one or more ring carbons or heteroatoms. In some embodiments, “heterocycloalkyl” encompasses 4- to 8-membered heterocycloalkyl, 5- to 8-membered heterocycloalkyl, 6- to 8-membered heterocycloalkyl, 7- to 8-membered heterocycloalkyl, 4- to 7-membered heterocycloalkyl, 5- to 7-membered heterocycloalkyl, 6 to 7-membered heterocycloalkyl, 4- to 6-membered heterocycloalkyl, 5- to 6-membered heterocycloalkyl, or 4- to 5-membered heterocycloalkyl. The term “alkylene” by itself or as part of another substituent means a divalent radical derived from an alkane, as exemplified by —CH₂CH₂CH₂CH₂—. Typically, an alkyl (or alkylene) group will have from 1 to 24 carbon atoms. In some embodiments, an alkyl (or alkylene) group will have 10 or fewer carbon atoms.

The term “heteroalkylene” by itself or as part of another substituent means a divalent radical, saturated or unsaturated or polyunsaturated, derived from heteroalkyl, as exemplified by —CH₂—CH₂—S—CH₂CH₂—, —CH₂—S—CH₂—CH₂—NH—CH₂—, —CH₂—CH(H)CH₂—O—CH₂— and —S—CH₂—C≡C—. For heteroalkylene groups, heteroatoms can also occupy either or both of the chain termini (e.g., alkyleneoxy, alkylenedioxy, alkyleneamino, alkylenediamino, and the like).

The term “heterocycloalkylene” by itself or as part of another substituent means a divalent radical, saturated or unsaturated or polyunsaturated, derived from heterocycloalkyl. For heterocycloalkylene groups, heteroatoms can also occupy either or both of the chain termini.

The terms “alkoxy” and “alkylamino” are used in their conventional sense, and refer to those alkyl groups attached to the remainder of the molecule via an oxygen atom or an amino group, respectively.

The term “heterocycloalkoxy” refers to a heterocycloalkyl-O— group in which the heterocycloalkyl group is as previously described herein.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “C₁-C₄ haloalkyl” is mean to include trifluoromethyl, 2,2,2-trifluoroethyl, 4-chlorobutyl, 3-bromopropyl, difluoromethyl, and the like.

The term “haloalkyl-OH” refers to a haloalkyl group as described above which is also substituted by one or more hydroxyl groups. The term “haloalkyl-OH” is meant to include haloalkyl substituted by one hydroxyl group, as well as haloalkyl substituted by multiple hydroxyl groups. For example, the term “haloalkyl-OH” includes —CH(F)OH, —CH₂CFHCH₂OH, —CH(OH)CF₃, and the like.

The term “alkyl-OH” refers to an alkyl substituted by one or more hydroxyl groups. The term “alkyl-OH” is meant to include alkyl substituted by one hydroxyl group, as well as alkyl substituted by multiple hydroxyl groups. For example, the term “alkyl-OH” includes —CH₂OH, —CH(OH)CH₃, —CH₂CH₂OH, and the like.

The term “aryl” means, unless otherwise stated, a polyunsaturated, typically aromatic, hydrocarbon group, which can be a single ring or multiple rings (up to three rings) which are fused together. In some embodiments, “aryl” encompasses C₆-C₁₄ aryl, C₈-C₁₄ aryl, C₁₀-C₁₄ aryl, C₁₂-C₁₄ aryl, C₆-C₁₂ aryl, C₈-C₁₂ aryl, C₁₀-C₁₂ aryl, C₆-C₁₀ aryl, C₈-C₁₀ aryl, or C₆-C₈ aryl. The term “heteroaryl” refers to aryl groups (or rings) that contain from one to five heteroatoms selected from the group consisting of N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl groups include phenyl, naphthyl and biphenyl, while non-limiting examples of heteroaryl groups include pyridyl, pyridazinyl, pyrazinyl, pyrimindinyl, triazinyl, quinolinyl, quinoxalinyl, quinazolinyl, cinnolinyl, phthalaziniyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like. In some embodiments, the term “heteroaryl” encompasses 5- to 10-membered heteroaryl, 6- to 10-membered heteroaryl, 7- to 10-membered heteroaryl, 8- to 10-membered heteroaryl, 9- to 10-membered heteroaryl, 5- to 9-membered heteroaryl, 6- to 9-membered heteroaryl, 7- to 9-membered heteroaryl, 8- to 9-membered heteroaryl, 5- to 8-membered heteroaryl, 6- to 8-membered heteroaryl, 7- to 8-membered heteroaryl, 5- to 7-membered heteroaryl, 6- to 7-membered heteroaryl, or 5- to 6-membered heteroaryl.

The above terms (e.g., “alkyl,” “aryl” and “heteroaryl”), in some embodiments, will include both substituted and unsubstituted forms of the indicated radical.

As used herein, the term “heteroatom” is meant to include oxygen (O), nitrogen (N), sulfur (S), boron (B), and silicon (Si).

As used herein, the term “chiral” refers to molecules which have the property of non-superimposability of the mirror image partner, while the term “achiral” refers to molecules which are superimposable on their mirror image partner.

As used herein, the term “stereoisomers” refers to compounds which have identical chemical constitution, but differ with regard to the arrangement of the atoms or groups in space.

As used herein, a wavy line “ [symbol] ” that intersects a bond in a chemical structure indicates the point of attachment of the atom to which the wavy bond is connected in the chemical structure to the remainder of a molecule, or to the remainder of a fragment of a molecule.

As used herein, the representation of a group (e.g., X^{a}) in parenthesis followed by a subscript integer range (e.g., (X^{a})₀₋₁) means that the group can have the number of occurrences as designated by the integer range. For example, (X^{a})₀₋₁ means the group X^{a} can be absent or can occur one time.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers can separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the present disclosure can contain asymmetric or chiral centers, and therefore exist in different stereoisomeric forms. It is intended that all stereoisomeric forms of the compounds of the present disclosure, including but not limited to, diastereomers, enantiomers and atropisomers, as well as mixtures thereof such as racemic mixtures, form part of the present disclosure. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

As used herein, the term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

As used herein, the term “solvate” refers to an association or complex of one or more solvent molecules and a compound of the present disclosure. Examples of solvents that form solvates include, but are not limited to, water, isopropanol, ethanol, methanol, DMSO, ethyl acetate, acetic acid, and ethanolamine. The term “hydrate” refers to the complex where the solvent molecule is water. Certain compounds of the present disclosure can exist in unsolvated forms as well as solvated forms, including hydrated forms. In general, the solvated forms are equivalent to unsolvated forms and are intended to be encompassed within the scope of the present disclosure.

The term “co-crystal” as used herein refers to a solid that is a crystalline single phase material composed of two or more different molecular or ionic compounds generally in a stoichiometric ratio which are neither solvates nor simple salts. A co-crystal consists of two or more components that form a unique crystalline structure having unique properties. Co-crystals are typically characterized by a crystalline structure, which is generally held together by freely reversible, non-covalent interactions. As used herein, a co-crystal refers to a compound of the present disclosure and at least one other component in a defined stoichiometric ratio that form a crystalline structure.

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4th edition, Wiley-Interscience, New York, 2006.

As used herein, the term “pharmaceutically acceptable salts” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

The neutral forms of the compounds can be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

Certain compounds of the present disclosure possess asymmetric carbon atoms (optical centers) or double bonds; the racemates, diastereomers, geometric isomers, regioisomers and individual isomers (e.g., separate enantiomers) are all intended to be encompassed within the scope of the present disclosure.

The compounds of the present disclosure can also contain unnatural proportions of atomic isotopes at one or more of the atoms that constitute such compounds. For example, the present disclosure also embraces isotopically-labeled variants of the present disclosure which are identical to those recited herein, but for the fact that one or more atoms are replaced by an atom having the atomic mass or mass number different from the predominant atomic mass or mass number usually found in nature for the atom. All isotopes of any particular atom or element as specified are contemplated within the scope of the compounds of the present disclosure and include isotopes of hydrogen, carbon, nitrogen, oxygen, phosphorous, sulfur, fluorine, chlorine and iodine, such as ²H (“D”), ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Certain isotopically labeled compounds of the present disclosure (e.g., those labeled with 3H or 14C) are useful in compound and/or substrate tissue distribution assays. Tritiated (³H) and carbon-14 (¹⁴C) isotopes are useful for their ease of preparation and detectability. Further substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labeled compounds of the present disclosure can generally be prepared by following procedures analogous to those disclosed in the Schemes and/or in the Examples herein below, by substituting an isotopically labeled reagent for a non-isotopically labeled reagent.

“Treating” or “treatment” of a disease in a patient refers to inhibiting the disease or arresting its development; or ameliorating or causing regression of the disease. As used herein, “treatment” or “treating” is an approach for obtaining beneficial or desired results including clinical results. For purposes of this disclosure, beneficial or desired results include, but are not limited to, one or more of the following: decreasing one more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delay or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of the present disclosure contemplate any one or more of these aspects of treatment.

“Preventing”, “prevention”, or “prophylaxis” of a disease in a patient refers to preventing the disease from occurring in a patient that is predisposed or does not yet display symptoms of the disease.

The phrase “therapeutically effective amount” means an amount of a compound of the present disclosure that (i) i treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The terms “cancer” and “cancerous” refer to or describe the physiological condition in mammals that is typically characterized by unregulated cell growth.

It is appreciated that certain features of the present disclosure, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the chemical groups represented by the variables are specifically embraced by the present invention and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present invention and are disclosed herein just as if each and every such sub-combination of chemical groups was individually and explicitly disclosed herein.

### II. Compounds

In one aspect, provided herein is a compound of formula (I)

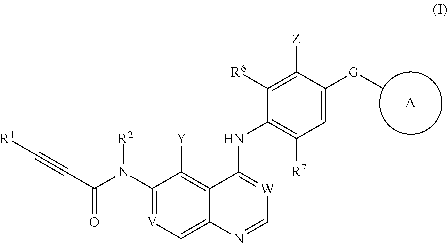

or a pharmaceutically acceptable salt thereof, wherein:
ring A is

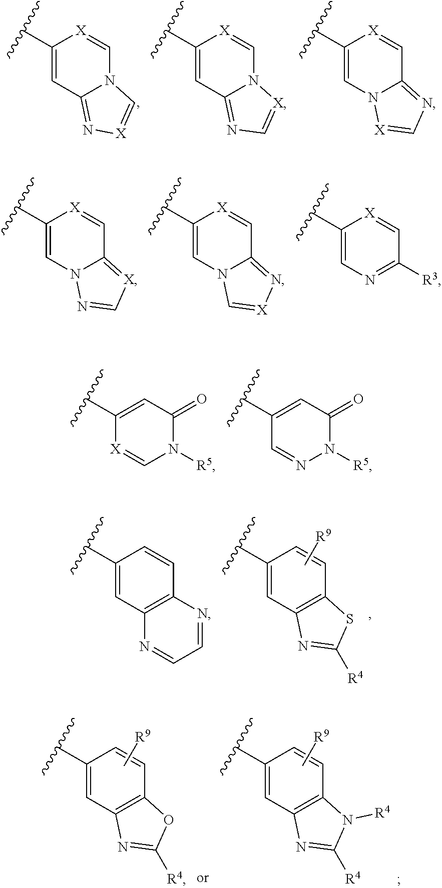

V is N or C—R⁸;
W is N or C—CN;
each X is independently N or CH;
G is CH₂, CH(CH₃),

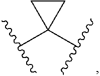 [SMILES: *C1(*)CC1]

O, or S;
Y is H, F, or —O(C₁-C₃ alkyl);
Z is H, F, Cl, C₁-C₂ alkyl, or cyclopropyl;
R¹ is H, C₁-C₄ alkyl, C₃-C₆ cycloalkyl, or 4- to 6-membered heterocyclyl containing 1-2 ring heteroatoms selected from the group consisting of N, O and S,
   wherein the C₁-C₃ alkyl is optionally substituted by 1-3 substituents selected from the group consisting of F, —OH, —OPO₃²⁻, —N(C₁-C₃ alkyl)(C₁-C₃ alkyl), and 4- to 6-membered heterocyclyl containing 1-2 ring heteroatoms selected from the group consisting of N and O; and wherein the C₃-C₆ cycloalkyl and 4- to 6-membered heterocyclyl are optionally substituted by 1-3 substituents selected from the group consisting of F, —OH, C₁-C₃ alkyl, —N(C₁-C₃ alkyl)(C₁-C₃ alkyl), and 4- to 6-membered heterocyclyl containing 1-2 ring heteroatoms selected from the group consisting of N and O;
R² is H, C₁-C₃ alkyl or cyclopropyl;
R³ is H, —CD₃, C₁-C₃ alkyl, —CF₂H, —CF₃, —CFH—CFH₂, allyl, —CH₂-cyclopropyl, cyclopropyl, —CN, —OR⁴, —SR⁴, —S(O)(C₁-C₃ alkyl), or —S(O)₂(C₁-C₃ alkyl);
each R⁴ is independently H, C₁-C₃ alkyl, —CF₂H, —CF₃, or cyclopropyl;
R⁵ is C₁-C₃ alkyl, —CD₃, —CF₂H, —CF₃, allyl, —CH₂-cyclopropyl, cyclopropyl, or —OR⁴;
R⁶ is H or F;
R⁷ is H or F;
R⁸ is H or —O(C₁-C₃ alkyl), wherein the C₁-C₃ alkyl is optionally substituted by 1-4 substituents selected from the group consisting of —F, —OH, —OR^{8a} and —NR^{8a}R^{8b}, wherein each R^{8a} and R^{8b} are independently H or C₁-C₃ alkyl or wherein each pair of geminal R^{8a} and R^{8b} may be taken together with the nitrogen atom to which they are attached to form an N-heterocycloalkyl wherein the N-heterocycloalkyl is optionally substituted by C₁-C₃ alkyl; and
R⁹ is H, halogen, C₁-C₃ alkyl, —CF₂H, —CF₃, cyclopropyl, —CN or —OR⁴.

In some embodiments wherein V is C-le, R⁸ is H and W is N, the compound of formula (I) is a compound of formula (II)

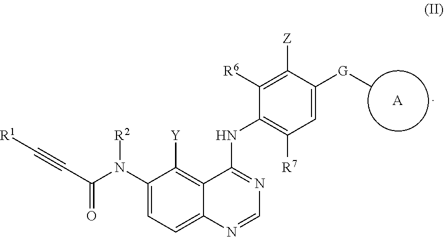

In another aspect, provided herein is a compound of formula (II)

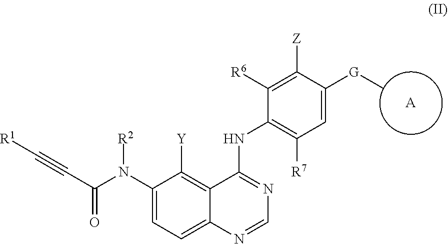

or a pharmaceutically acceptable salt thereof, wherein:
ring A is

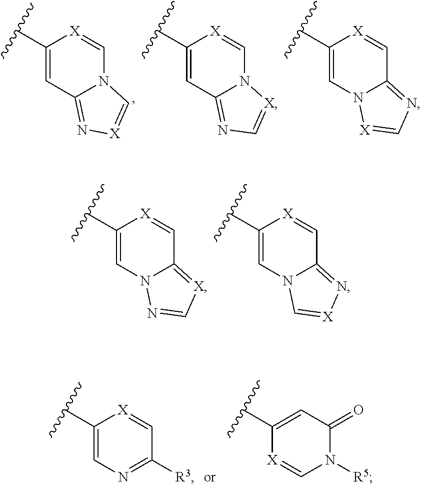

each X is independently N or CH;
G is CH₂, O, or S;
Y is H, F, or —O(C₁-C₃ alkyl);
Z is H, F, Cl, C₁-C₂ alkyl, or cyclopropyl;
R¹ is H, C₁-C₃ alkyl, C₃-C₆ cycloalkyl, or 4- to 6-membered heterocyclyl containing 1-2 ring heteroatoms selected from the group consisting of N, O and S,
   wherein the C₁-C₃ alkyl, C₃-C₆ cycloalkyl, and 4- to 6-membered heterocyclyl are optionally substituted by 1-3 substituents selected from the group consisting of F, —OH, —N(C₁-C₃ alkyl)(C₁-C₃ alkyl), and 4- to 6-membered heterocyclyl containing 1-2 ring heteroatoms selected from the group consisting of N and O;
R² is H, C₁-C₃ alkyl or cyclopropyl;
R³ is H, —CD₃, C₁-C₃ alkyl, —CF₂H, —CF₃, allyl, —CH₂-cyclopropyl, cyclopropyl, —CN, —SR⁴, —S(O)(C₁-C₃ alkyl), or —S(O)₂(C₁-C₃ alkyl);
R⁴ is H, C₁-C₃ alkyl, —CF₂H, —CF₃, or cyclopropyl;
R⁵ is C₁-C₃ alkyl, —CD₃, —CF₂H, —CF₃, allyl, —CH₂-cyclopropyl, cyclopropyl, or —OR⁴;
R⁶ is H or F; and
R⁷ is H or F.

In some embodiments, ring A is

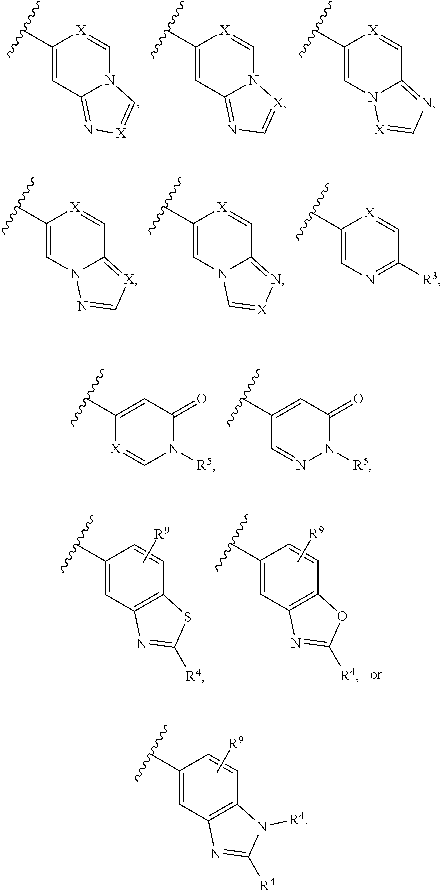

In some embodiments, ring A is

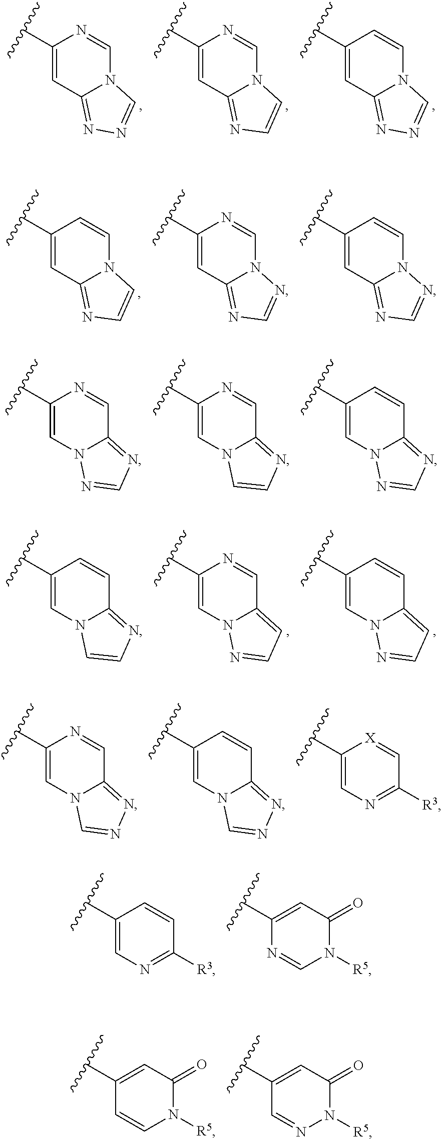

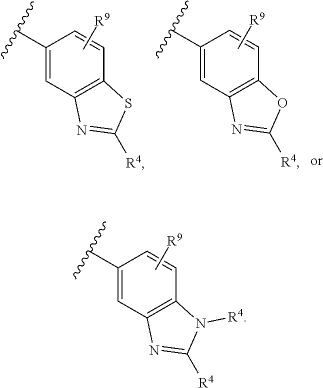

In some embodiments, ring A is

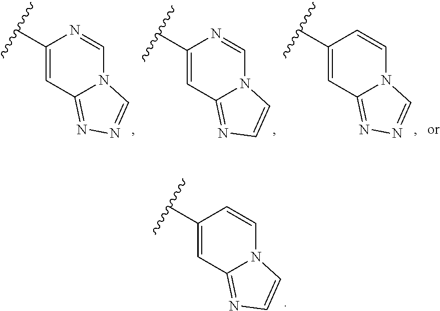 [SMILES: *c1cc2nncn2cn1 ; *c1cc2nccn2cn1 ; *c1ccn2cnnc2c1 ; *c1ccn2ccnc2c1]

In some embodiments, ring A is

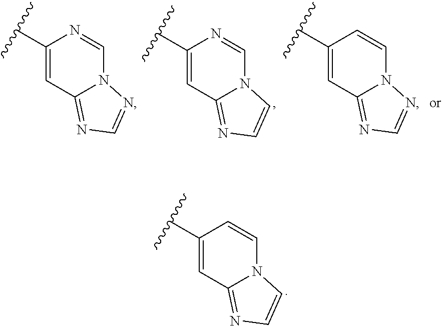 [SMILES: *c1cc2ncnn2cn1 ; *c1cc2nccn2cn1 ; *c1ccn2ncnc2c1 ; *c1ccn2ccnc2c1]

In some embodiments, ring A is

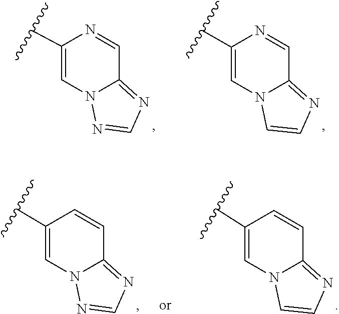 [SMILES: *c1cn2ncnc2cn1 ; *c1cn2ccnc2cn1 ; *c1ccc2ncnn2c1 ; *c1ccc2nccn2c1]

In some embodiments, ring A is

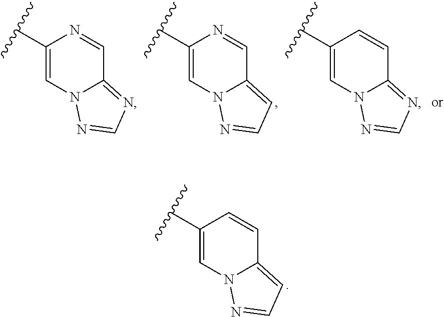 [SMILES: *c1cn2ncnc2cn1 ; *c1cn2nccc2cn1 ; *c1ccc2ncnn2c1 ; *c1ccc2ccnn2c1]

In some embodiments, ring A is

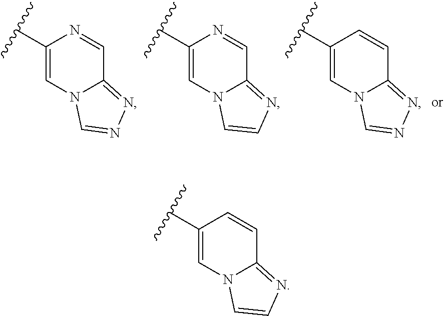 [SMILES: *c1cn2cnnc2cn1 ; *c1cn2ccnc2cn1 ; *c1ccc2nncn2c1 ; *c1ccc2nccn2c1]

In other embodiments, ring A is

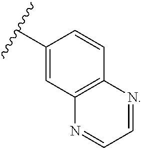 [SMILES: *c1ccc2nccnc2c1]

In other embodiments, ring A is

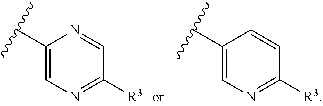

In some embodiments, ring A is

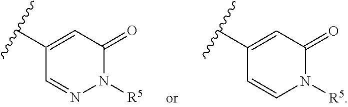

In some embodiments, ring A is

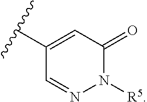

In some embodiments ring A is

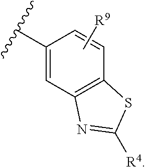

In some embodiments ring A is

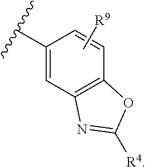

In some embodiments, ring A is

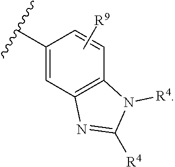

In some embodiments, ring A is

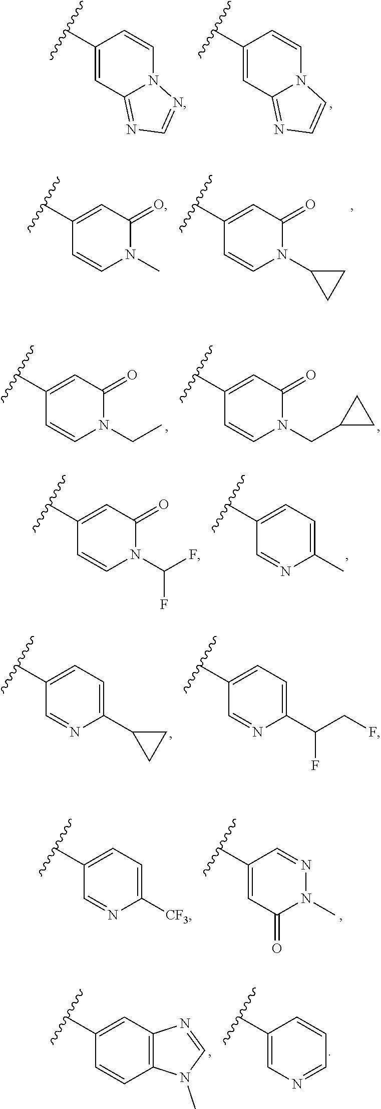

In some embodiments, ring A is

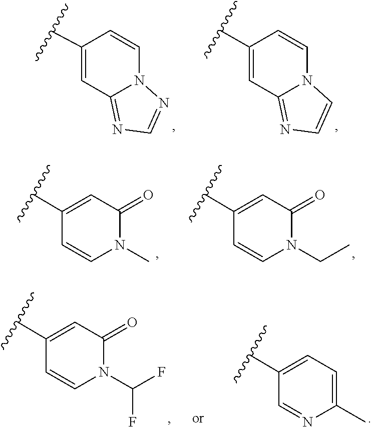

In some embodiments, ring A is

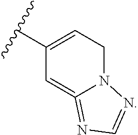

In some embodiments, ring A is

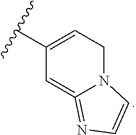

In some embodiments, ring A is

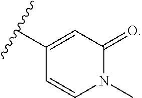 [SMILES: *c1ccn(C)c(=O)c1]

In some embodiments, ring A is

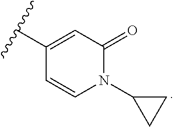 [SMILES: *c1ccn(C2CC2)c(=O)c1]

In some embodiments, ring A is

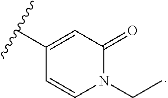 [SMILES: *c1ccn(CC)c(=O)c1]

In some embodiments, ring A is

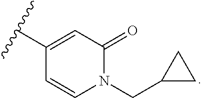 [SMILES: *c1ccn(CC2CC2)c(=O)c1]

In some embodiments, ring A is

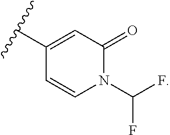 [SMILES: *c1ccn(C(F)F)c(=O)c1]

In some embodiments, ring A is

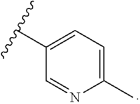 [SMILES: *c1ccc(C)nc1]

In some embodiments, ring A is

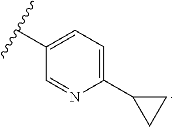 [SMILES: *c1ccc(C2CC2)nc1]

In some embodiments, ring A is

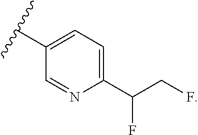 [SMILES: *c1ccc(C(F)CF)nc1]

In some embodiments, ring A is

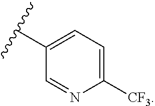 [SMILES: *c1ccc(C(F)(F)F)nc1]

In some embodiments, ring A is

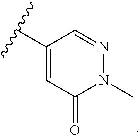 [SMILES: *c1cnn(C)c(=O)c1]

In some embodiments ring A is

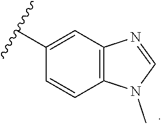 [SMILES: *c1ccc2c(c1)ncn2C]

In some embodiments, ring A is

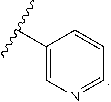 [SMILES: *c1cccnc1]

In some embodiments ring A is

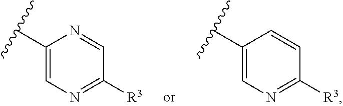

and R³ is H, —CD₃, C₁-C₃ alkyl, —CF₂H, —CF₃, —CFH—CFH₂, allyl, —CH₂-cyclopropyl, cyclopropyl, —CN, —OR⁴, —S(O)(C₁-C₃ alkyl), or —S(O)₂(C₁-C₃ alkyl). In some embodiments, ring A is

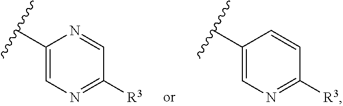

and R³ is H, —CD₃, C₁-C₃ alkyl, —CF₂H, —CF₃, allyl, —CH₂— cyclopropyl, cyclopropyl, —CN, —SR⁴, —S(O)(C₁-C₃ alkyl), or —S(O)₂(C₁-C₃ alkyl). In some embodiments, R³ is H, —CD₃, C₁-C₂ alkyl, CF₂H, CF₃, —CFH—CFH₂, allyl, —CH₂— cyclopropyl, cyclopropyl, —CN, —SR⁴, —S(O)(C₁-C₂ alkyl), or —S(O)₂(C₁-C₂ alkyl). In some embodiments, R³ is H, —CD₃, C₁-C₂ alkyl, CF₂H, CF₃, allyl, —CH₂-cyclopropyl, cyclopropyl, —CN, —SR⁴, —S(O)(C₁-C₂ alkyl), or —S(O)₂(C₁-C₂ alkyl). In some embodiments, R³ is H, —CD₃, —CH₃, —CF₂H, —CF₃, allyl, —CH₂-cyclopropyl, cyclopropyl, —CN, —SR⁴, —S(O)(CH₃), or —S(O)₂(CH₃). In some embodiments, R³ is —CH₃, —CF₃, —CFH—CFH₂, or —CD₃. In some embodiments, R³ is —CH₃ or —CD₃. In some embodiments, R³ is C₁-C₃ alkyl. In some embodiments, R³ is methyl, ethyl, n-propyl, or isopropyl. In some embodiments, R³ is —CFH—CFH₂. In some embodiments, R³ is —CF₃. In some embodiments, R³ is allyl. In some embodiments, R³ is —CH₂-cyclopropyl. In some embodiments, R³ is cyclopropyl. In some embodiments, R³ is —S(O)(methyl), —S(O)(ethyl), —S(O)(n-propyl), or —S(O)(isopropyl). In some embodiments, R³ is —S(O)₂(methyl), —S(O)₂(ethyl), —S(O)₂(n-propyl), or —S(O)₂(isopropyl).

In some embodiments, ring A is

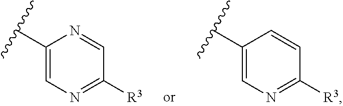

R³ is —OR⁴ or —SR⁴, and R⁴ is H, C₁-C₃ alkyl, —CF₂H, —CF₃, or cyclopropyl. In some embodiments, R³ is —OR⁴ or —SR⁴, and R⁴ is H, C₁-C₂ alkyl, —CF₂H, —CF₃, or cyclopropyl. In some embodiments, R³ is —OR⁴ or —SR⁴, and R⁴ is H, —CH₃, —CHF₂, or cyclopropyl. In some embodiments, R³ is —OH or —SH. In some embodiments, R³ is —O(C₁-C₃ alkyl) or —S(C₁-C₃ alkyl). In some embodiments, R³ is —O(methyl), —O(ethyl), —O(n-propyl), —O(isopropyl), —S(methyl), —S(ethyl), —S(n-propyl), or —S(isopropyl). In some embodiments, R³ is —O(cyclopropyl). In some embodiments, R³ is —O(CF₂H) or —S(CF₂H). In some embodiments, R³ is —O(CF₃) or —S(CF₃).

In some embodiments, ring A is

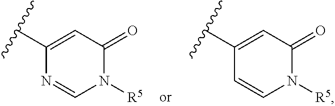

and R⁵ is C₁-C₃ alkyl, —CD₃, —CF₂H, —CF₃, allyl, —CH₂-cyclopropyl, cyclopropyl, or —OR⁴. In some embodiments, R⁵ is C₁-C₂ alkyl, —CD₃, CF₂H, CF₃, allyl, —CH₂-cyclopropyl, cyclopropyl, or —OR⁴. In some embodiments, R⁵ is —CH₃ or —CD₃. In some embodiments, R⁵ is C₁-C₃ alkyl. In some embodiments, R⁵ is methyl, ethyl, n-propyl, or isopropyl. In some embodiments, R⁵ is —CH₂-cyclopropyl or cyclopropyl. In some embodiments, R⁵ is cyclopropyl.

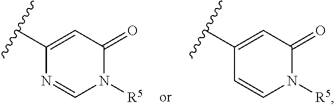

In some embodiments, ring A is R⁵ is —OR⁴, and R⁴ is H, C₁-C₃ alkyl, —CF₂H, —CF₃, or cyclopropyl. In some embodiments, R⁵ is —OR⁴, and R⁴ is H, C₁-C₂ alkyl, —CF₂H, —CF₃, or cyclopropyl. In some embodiments, R⁵ is —OR⁴, and R⁴ is H, —CH₃, —CHF₂, or cyclopropyl. In some embodiments, R⁵ is —OH. In some embodiments, R⁵ is —O(C₁-C₃ alkyl). In some embodiments, R⁵ is —O(methyl), —O(ethyl), —O(n-propyl), or —O(isopropyl). In some embodiments, R⁵ is —O(cyclopropyl). In some embodiments, R⁵ is —O(CF₂H). In some embodiments, R⁵ is —O(CF₃).

In some embodiments, ring A is

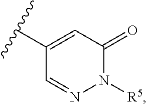

R⁵ is —OR⁴, and R⁴ is H, C₁-C₃ alkyl, —CF₂H, —CF₃, or cyclopropyl. In some embodiments, R⁵ is —OR⁴, and R⁴ is H, C₁-C₂ alkyl, —CF₂H, —CF₃, or cyclopropyl. In some embodiments, R⁵ is —OR⁴, and R⁴ is H, —CH₃, —CHF₂, or cyclopropyl. In some embodiments, R⁵ is —OH. In some embodiments, R⁵ is —O(C₁-C₃ alkyl). In some embodiments, R⁵ is —O(methyl), —O(ethyl), —O(n-propyl), or —O(isopropyl). In some embodiments, R⁵ is —O(cyclopropyl). In some embodiments, R⁵ is —O(CF₂H). In some embodiments, R⁵ is —O(CF₃).

In some embodiments, ring A is

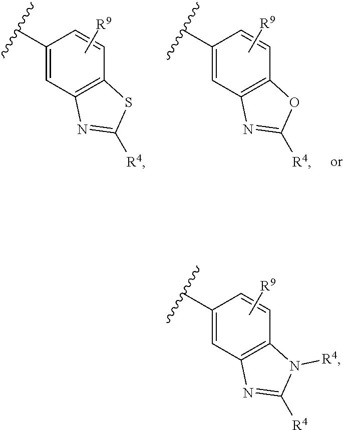

and each R⁴ is independently H, C₁-C₃ alkyl, —CF₂H, —CF₃, or cyclopropyl. In some embodiments, each R⁴ is independently H, C₁-C₂ alkyl, —CF₂H, —CF₃, or cyclopropyl. In some embodiments, each R⁴ is independently H, —CH₃, —CHF₂, or cyclopropyl. In some embodiments, each R⁴ is independently H. In some embodiments, each R⁴ is independently (C₁-C₃ alkyl). In some embodiments, each R⁴ is independently methyl, ethyl, -n-propyl, or -isopropyl. In some embodiments, R⁴ is cyclopropyl. In some embodiments, R⁴ is CF₂H. In some embodiments, R⁴ is CF₃.

In some embodiments, ring A is

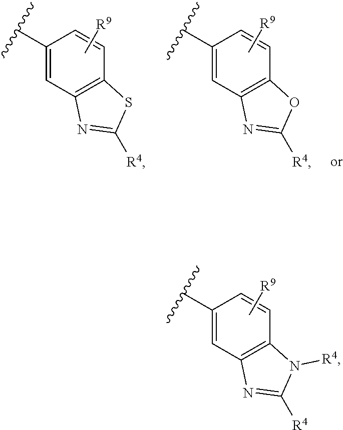

and R⁹ is H, halogen, C₁-C₃ alkyl, —CF₂H, —CF₃, cyclopropyl, —CN or —OR⁴. In some embodiments, R⁹ is H, halogen C₁-C₂ alkyl CF₂H, —CF₃, cyclopropyl, —CN or —OR⁴. In some embodiments, R⁹ is H. In some embodiments, R⁹ is halogen. In some embodiments, R⁹ is C₁-C₃ alkyl. In some embodiments, R⁹ is methyl, ethyl, n-propyl, or isopropyl. In some embodiments, R⁹ is cyclopropyl. In some embodiments, R⁹ is —CN. In some embodiments, R⁹ is —OR⁴.

In some embodiments, ring A is

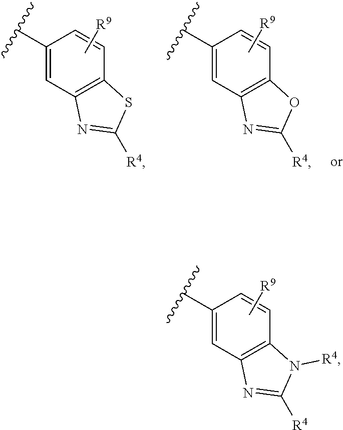

R⁹ is —OR⁴, and each R⁴ is independently H, C₁-C₃ alkyl, —CF₂H, —CF₃, or cyclopropyl. In some embodiments, R⁹ is —OR⁴, and each R⁴ is independently H, C₁-C₂ alkyl, —CF₂H, —CF₃, or cyclopropyl. In some embodiments, R⁹ is —OR⁴, and each R⁴ is independently H, —CH₃, —CHF₂, or cyclopropyl. In some embodiments, R⁹ is —OH. In some embodiments, R⁹ is —O(C₁-C₃ alkyl). In some embodiments, R⁹ is —O(methyl), —O(ethyl), —O(n-propyl), or —O(isopropyl). In some embodiments, R⁹ is —O(cyclopropyl). In some embodiments, R⁹ is —O(CF₂H). In some embodiments, R⁹ is —O(CF₃).

In some embodiments, G is CH₂, CH(CH₃),

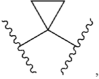 [SMILES: *C1(*)CC1]

O, or S. In some embodiments, G is CH₂, O, or S. In some embodiments, G is CH₂ or O. In some embodiments, G is CH₂ or S. In some embodiments, G is O or S. In some embodiments, G is CH₂. In some embodiments, G is CH(CH₃). In some embodiments, G is

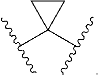 [SMILES: *C1(*)CC1]

In some embodiments, G is O. In some embodiments, G is S.

In some embodiments, Z is H, F, Cl, C₁-C₂ alkyl, or cyclopropyl. In some embodiments, Z is H, F, Cl, or —CH₃. In some embodiments, Z is —CH₃. In some embodiments, Z is H. In some embodiments, Z is F. In some embodiments, Z is C₁. In some embodiments, Z is —CH₂CH₃. In some embodiments, Z is cyclopropyl.

In some embodiments, R¹ is H, C₁-C₄ alkyl, C₃-C₆ cycloalkyl, or 4- to 6-membered heterocyclyl containing 1-2 ring heteroatoms selected from the group consisting of N, O and S, wherein the C₁-C₃ alkyl is optionally substituted by 1-3 substituents selected from the group consisting of F, —OH, —OPO₃²—, —N(C₁-C₃ alkyl)(C₁-C₃ alkyl), and 4- to 6-membered heterocyclyl containing 1-2 ring heteroatoms selected from the group consisting of N and O; and wherein the C₃-C₆ cycloalkyl and 4- to 6-membered heterocyclyl are optionally substituted by 1-3 substituents selected from the group consisting of F, —OH, C₁-C₃ alkyl, —N(C₁-C₃ alkyl)(C₁-C₃ alkyl), and 4- to 6-membered heterocyclyl containing 1-2 ring heteroatoms selected from the group consisting of N and O. In some embodiments, R¹ is H, C₁-C₃ alkyl, C₃-C₆ cycloalkyl, or 4- to 6-membered heterocyclyl containing 1-2 ring heteroatoms selected from the group consisting of N, O and S, wherein the C₁-C₃ alkyl, C₃-C₆ cycloalkyl, and 4- to 6-membered heterocyclyl are optionally substituted by 1-3 substituents selected from the group consisting of F, —OH, —N(C₁-C₃ alkyl)(C₁-C₃ alkyl), and 4- to 6-membered heterocyclyl containing 1-2 ring heteroatoms selected from the group consisting of N and O.

In some embodiments, R¹ is H.

In some embodiments, R¹ is C₁-C₄ alkyl optionally substituted by 1-3 substituents selected from the group consisting of F, —OH, —N(C₁-C₃ alkyl)(C₁-C₃ alkyl), and 4- to 6-membered heterocyclyl containing 1-2 ring heteroatoms selected from the group consisting of N and O. In some embodiments, R¹ is C₁-C₃ alkyl optionally substituted by 1-3 substituents selected from the group consisting of F, —OH, —N(C₁-C₃ alkyl)(C₁-C₃ alkyl), and 4- to 6-membered heterocyclyl containing 1-2 ring heteroatoms selected from the group consisting of N and O. In some embodiments, R¹ is C₁-C₃ alkyl substituted by a 4- to 6-membered heterocyclyl containing 1-2 ring heteroatoms selected from the group consisting of N and O, wherein the 4- to 6-membered heterocyclyl is optionally substituted by 1-3 substituents selected from the group consisting of F, —OH, C₁-C₃ alkyl, —N(C₁-C₃ alkyl)(C₁-C₃ alkyl).

In some embodiments, R¹ is unsubstituted C₁-C₄ alkyl. In some embodiments, IV is unsubstituted C₁-C₃ alkyl. In some embodiments, R¹ is methyl, ethyl, n-propyl, isopropyl, n-butyl, sec-butyl, isobutyl, or t-butyl, each of which is unsubstituted. In some embodiments, IV is methyl, ethyl, n-propyl, or isopropyl, each of which is unsubstituted. In some embodiments, R¹ is —CH₃. In some embodiments, R¹ is n-butyl, sec-butyl, isobutyl, or t-butyl, each of which is unsubstituted. In some embodiments, R¹ is t-butyl.

In some embodiments, R¹ is C₁-C₃ alkyl substituted by 1-3 substituents selected from the group consisting of F, —OH, —OPO₃²—, —N(C₁-C₃ alkyl)(C₁-C₃ alkyl), and 4- to 6-membered heterocyclyl containing 1-2 ring heteroatoms selected from the group consisting of N and O. In some embodiments, R¹ is C₁-C₂ alkyl substituted by 1-3 substituents selected from the group consisting of F, —OH, —OPO₃²—, —N(C₁-C₃ alkyl)(C₁-C₃ alkyl), and 4- to 6-membered heterocyclyl containing 1-2 ring heteroatoms selected from the group consisting of N and O. In some embodiments, R¹ is methyl, ethyl, n-propyl, or isopropyl, each of which is substituted by 1-3 substituents selected from the group consisting of F, —OH, —OPO₃²—, —N(C₁-C₃ alkyl)(C₁-C₃ alkyl), and 4- to 6-membered heterocyclyl containing 1-2 ring heteroatoms selected from the group consisting of N and O. In some embodiments, R¹ is C₁-C₂ alkyl optionally substituted by —OH, —OPO₃²—, or —N(C₁-C₃ alkyl)(C₁-C₃ alkyl). In still other embodiments, R¹ is —(CH₂)₂OPO₃²—.

In some embodiments, R¹ is C₁-C₃ alkyl substituted by 1-3 substituents selected from the group consisting of F, —OH, —N(C₁-C₃ alkyl)(C₁-C₃ alkyl), and 4- to 6-membered heterocyclyl containing 1-2 ring heteroatoms selected from the group consisting of N and O. In some embodiments, R¹ is C₁-C₂ alkyl substituted by 1-3 substituents selected from the group consisting of F, —OH, —N(C₁-C₃ alkyl)(C₁-C₃ alkyl), and 4- to 6-membered heterocyclyl containing 1-2 ring heteroatoms selected from the group consisting of N and O. In some embodiments, R¹ is methyl, ethyl, n-propyl, or isopropyl, each of which is substituted by 1-3 substituents selected from the group consisting of F, —OH, —N(C₁-C₃ alkyl)(C₁-C₃ alkyl), and 4- to 6-membered heterocyclyl containing 1-2 ring heteroatoms selected from the group consisting of N and O.

In some embodiments, R¹ is methyl, ethyl, n-propyl, or isopropyl, each of which is substituted by 1-3 F groups. In some embodiments, R¹ is —CH₂F, —CHF₂, —CF₃, —CH₂CH₂F, —CH₂CHF₂, —CH₂CF₃, —CHFCH₃, —CF₂CH₃, —CHFCH₂F, —CF₂CH₂F, —CHFCHF₂, —CH₂CH₂CH₂F, —CH₂CH₂CHF₂, —CH₂CH₂CF₃, —CH₂CHFCH₂F, —CH₂CHFCHF₂, —CH₂CF₂CH₂F, —CHFCHFCH₂F, —CF₂CH₂CH₂F, —CHFCH₂CH₃, —CH₂CHFCH₃, —CHFCHFCH₃, —CHFCF₂CH₃, or —CF₂CHFCH₃.

In some embodiments, R¹ is methyl, ethyl, n-propyl, or isopropyl, each of which is substituted by 1-3 —OH groups. In some embodiments, R¹ is methyl-OH, ethyl-OH, n-propyl-OH, or isopropyl-OH. In some embodiments, R¹ is —CH₂OH, —CH(OH)CH₃, —CH₂CH₂OH, —CH(OH)CH₂CH₃, —CH₂CH(OH)CH₃, —CH₂CH₂CH₂OH, —C(CH₃)₂OH, or —CH(CH₂OH)(CH₃).

In some embodiments, R¹ is methyl, ethyl, n-propyl, or isopropyl, each of which is substituted by —N(C₁-C₃ alkyl)(C₁-C₃ alkyl). In some embodiments, R¹ is methyl, ethyl, n-propyl, or isopropyl, each of which is substituted by —N(methyl)(methyl), —N(methyl)(ethyl), —N(methyl)(n-propyl), —N(methyl)(isopropyl), —N(ethyl)(ethyl), —N(ethyl)(n-propyl), —N(ethyl)(isopropyl), —N(n-propyl)(n-propyl), —N(n-propyl)(isopropyl), or —N(isopropyl)(isopropyl). In some embodiments, R¹ is methyl substituted by —N(methyl)(methyl), —N(methyl)(ethyl), —N(methyl)(n-propyl), —N(methyl)(isopropyl), —N(ethyl)(ethyl), —N(ethyl)(n-propyl), —N(ethyl)(isopropyl), —N(n-propyl)(n-propyl), —N(n-propyl)(isopropyl), or —N(isopropyl)(isopropyl). In some embodiments, R¹ is methyl, ethyl, n-propyl, or isopropyl, each of which is substituted by —N(methyl)(methyl). In some embodiments, R¹ is methyl substituted by —N(methyl)(methyl).

In some embodiments, R¹ is methyl, ethyl, n-propyl, or isopropyl, each of which is substituted by 4- to 6-membered heterocyclyl containing 1-2 ring heteroatoms selected from the group consisting of N and O. In some embodiments, R¹ is methyl, ethyl, n-propyl, or isopropyl, each of which is substituted by 4- to 6-membered heterocyclyl containing 1 nitrogen atom. In some embodiments, R¹ is methyl, ethyl, n-propyl, or isopropyl, each of which is substituted by 4- to 6-membered heterocyclyl containing 1 oxygen atom. In some embodiments, R¹ is methyl, ethyl, n-propyl, or isopropyl, each of which is substituted by 4- to 6-membered heterocyclyl containing 2 oxygen atoms. In some embodiments, R¹ is methyl, ethyl, n-propyl, or isopropyl, each of which is substituted by 4- to 6-membered heterocyclyl containing 2 nitrogen atoms. In some embodiments, R¹ is methyl, ethyl, n-propyl, or isopropyl, each of which is substituted by 4- to 6-membered heterocyclyl containing 1 oxygen atom and 1 nitrogen atom. In some embodiments, R¹ is methyl, ethyl, n-propyl, or isopropyl, each of which is substituted by azetidinyl, pyrrolidinyl, piperazinyl, piperidinyl, oxetanyl, tetrahydrofuranyl, or tetrahydropyranyl.

In some embodiments, R¹ is C₃-C₆ cycloalkyl optionally substituted by 1-3 substituents selected from the group consisting of F, —OH, C₁-C₃ alkyl, —N(C₁-C₃ alkyl)(C₁-C₃ alkyl), and 4- to 6-membered heterocyclyl containing 1-2 ring heteroatoms selected from the group consisting of N and O.

In some embodiments, R¹ is unsubstituted C₃-C₆ cycloalkyl. In some embodiments, R¹ is cyclopropyl, cyclobutyl, cyclopentyl, or cyclohexyl, each of which is unsubstituted. In some embodiments, R¹ is unsubstituted cyclopropyl.

In some embodiments, R¹ is C₃-C₆ cycloalkyl substituted by 1-3 substituents selected from the group consisting of F, —OH, C₁-C₃ alkyl, —N(C₁-C₃ alkyl)(C₁-C₃ alkyl), and 4- to 6-membered heterocyclyl containing 1-2 ring heteroatoms selected from the group consisting of N and O. In some embodiments, R¹ is cyclopropyl, cyclobutyl, cyclopentyl, or cyclohexyl, each of which is substituted by 1-3 substituents selected from the group consisting of F, —OH, C₁-C₃ alkyl, —N(C₁-C₃ alkyl)(C₁-C₃ alkyl), and 4- to 6-membered heterocyclyl containing 1-2 ring heteroatoms selected from the group consisting of N and O. In some embodiments, R¹ is cyclopropyl substituted by 1-3 substituents selected from the group consisting of F, —OH, C₁-C₃ alkyl, —N(C₁-C₃ alkyl)(C₁-C₃ alkyl), and 4- to 6-membered heterocyclyl containing 1-2 ring heteroatoms selected from the group consisting of N and O.

In some embodiments, R¹ is 4- to 6-membered heterocyclyl containing 1-2 ring heteroatoms selected from the group consisting of N, O and S, wherein the 4- to 6-membered heterocyclyl is optionally substituted by 1-3 substituents selected from the group consisting of F, —OH, C₁-C₃ alkyl, —N(C₁-C₃ alkyl)(C₁-C₃ alkyl), and 4- to 6-membered heterocyclyl containing 1-2 ring heteroatoms selected from the group consisting of N and O. In some embodiments, R¹ is 4- to 6-membered heterocyclyl containing 1 nitrogen atom. In some embodiments, R¹ is 4- to 6-membered heterocyclyl containing 1 oxygen atom. In some embodiments, R¹ is 4- to 6-membered heterocyclyl containing 1 sulfur atom. In some embodiments, R¹ is 4- to 6-membered heterocyclyl containing 2 nitrogen atoms. In some embodiments, R¹ is 4- to 6-membered heterocyclyl containing 2 oxygen atoms. In some embodiments, R¹ is 4- to 6-membered heterocyclyl containing 2 sulfur atoms. In some embodiments, R¹ is 4- to 6-membered heterocyclyl containing 1 nitrogen atom and 1 oxygen atom. In some embodiments, R¹ is 4- to 6-membered heterocyclyl containing 1 nitrogen atom and 1 sulfur atom. In some embodiments, R¹ is 4- to 6-membered heterocyclyl containing 1 oxygen atom and 1 sulfur atom. In some embodiments, R¹ is azetidinyl, pyrrolidinyl, piperidinyl, oxetanyl, tetrahydrofuranyl, tetrahydropyranyl, thietanyl, tetrahydrothiophenyl, or tetrahydrothiopyranyl.

In some embodiments, R¹ is unsubstituted 4- to 6-membered heterocyclyl containing 1-2 ring heteroatoms selected from the group consisting of N, O and S. In some embodiments, R¹ is azetidinyl, pyrrolidinyl, piperidinyl, oxetanyl, tetrahydrofuranyl, tetrahydropyranyl, thietanyl, tetrahydrothiophenyl, or tetrahydrothiopyranyl, each of which is unsubstituted.

In some embodiments, R¹ is 4- to 6-membered heterocyclyl containing 1-2 ring heteroatoms selected from the group consisting of N, O and S, wherein the 4- to 6-membered heterocyclyl is substituted by 1-3 substituents selected from the group consisting of F, —OH, C₁-C₃ alkyl, —N(C₁-C₃ alkyl)(C₁-C₃ alkyl), and 4- to 6-membered heterocyclyl containing 1-2 ring heteroatoms selected from the group consisting of N and O. In some embodiments, R¹ is 4- to 6-membered heterocyclyl containing 1 nitrogen atom. In some embodiments, R¹ is 4- to 6-membered heterocyclyl containing 1 oxygen atom. In some embodiments, R¹ is 4- to 6-membered heterocyclyl containing 1 sulfur atom. In some embodiments, R¹ is azetidinyl, pyrrolidinyl, piperidinyl, oxetanyl, tetrahydrofuranyl, tetrahydropyranyl, thietanyl, tetrahydrothiophenyl, or tetrahydrothiopyranyl, each of which is substituted by 1-3 substituents selected from the group consisting of F, —OH, C₁-C₃ alkyl, —N(C₁-C₃ alkyl)(C₁-C₃ alkyl), and 4- to 6-membered heterocyclyl containing 1-2 ring heteroatoms selected from the group consisting of N and O.

In some embodiments, Y is H, F, or —O(C₁-C₃ alkyl). In some embodiments, Y is H, F, or —O(C₁-C₂ alkyl). In some embodiments, Y is H, F, or —OCH₃. In some embodiments, Y is H. In some embodiments, Y is F. In some embodiments, Y is —O(C₁-C₃ alkyl). In some embodiments, Y is —O(methyl), —O(ethyl), —O(n-propyl), or —O(isopropyl).

In some embodiments, R² is H, C₁-C₃ alkyl or cyclopropyl. In some embodiments, R² is C₁-C₃ alkyl. In some embodiments, R² is H. In some embodiments, R² is methyl, ethyl, n-propyl, or isopropyl. In some embodiments, R² is cyclopropyl.

In some embodiments, R⁶ is H or F. In some embodiments, R⁶ is H. In some embodiments, R⁶ is F.

In some embodiments, R⁷ is H or F. In some embodiments, R⁷ is H. In some embodiments, R⁷ is F.

In some embodiments, both R⁶ and R⁷ are H. In some embodiments, both R⁶ and R⁷ are F. In some embodiments, R⁶ is H and R⁷ is F. In some embodiments, R⁶ is F and R⁷ is H.

In some embodiments, V is N or CR⁸. In some embodiments, V is N.

In some embodiments, V is CR⁸ and R⁸ is H or —O(C₁-C₃-alkyl), wherein the C₁-C₃-alkyl of the —O(C₁-C₃-alkyl) is optionally substituted by 1-4 substituents selected from the group consisting of —F, —OH, —OR^{8a}, and —NR^{8a}R^{8b}, wherein each R^{8a} and R^{8b} are independently H or C₁-C₃ alkyl or wherein each pair of geminal R^{8a} and R^{8b} may be taken together with the nitrogen atom to which they are attached to form an N-heterocycloalkyl wherein the N-heterocycloalkyl is optionally substituted by C₁-C₃ alkyl. In some embodiments, R⁸ is —H. In some embodiments, R⁸ is optionally substituted —O(C₁-C₃-alkyl).

In some embodiments, R⁸ is optionally substituted methoxy, ethoxy, n-propoxy, or isopropoxy. In some embodiments, R⁸ is —(O)C₁-C₃ alkyl. In some embodiments, R⁸ is —OCH₃, —OCH₂CH₃, —OCH₂CH₂CH₃, or —OCH(CH₃)₂. In certain embodiments, R⁸ is —OCH₃. In certain embodiments, R⁸ is —OCH₂CH₃. In certain embodiments, R⁸ is —OCH₂CH₂CH₃. In certain embodiments, R⁸ is —OCH(CH₃)₂.

In some embodiments, V is C R⁸ and R⁸ is —O(C₁-C₃ alkyl), wherein the —O(C₁-C₃ alkyl) is substituted by 1-4 substituents selected from the group consisting of —F, —OH, a 3- to 7-membered carbon-linked N-heterocycloalkyl, and —NR^{8a}R^{8b}, wherein each R^{8a} and R^{8b} are independently H or C₁-C₃ alkyl or wherein each pair of geminal R^{8a} and R^{8b} may be taken together with the nitrogen atom to which they are attached to form an N-heterocycloalkyl wherein the N-heterocycloalkyl is optionally substituted by C₁-C₃ alkyl.

In some embodiments, V is CR⁸ and R⁸ is —O(C₁-C₃-alkyl) substituted by 1 substituent selected from the group consisting of —F, —OH, —OR^{8a} and —NR^{8a}R^{8b}. In some embodiments, R⁸ is —O(C₁-C₃-alkyl) substituted by 2 substituents selected from the group consisting of —F, —OH, —OR^{8a} and —NR^{8a}R^{8b}. In some embodiments, R⁸ is —O(C₁-C₃-alkyl) substituted by 3 substituents selected from the group consisting of —F, —OH, —OR^{8a} and —NR^{8a}R^{8b}. In some embodiments, R⁸ is —O(C₁-C₃-alkyl) substituted by 4 substituents selected from the group consisting of —F, —OH, —OR^{8a} and —NR^{8a}R^{8b}.

In some embodiments, V is CR⁸ and R⁸ is —O(C₁-C₃-alkyl) substituted by —F. In some embodiments, R⁸ is methoxy, ethoxy, n-propoxy, or isopropoxy, each of which is substituted by 1-4 F groups. In some embodiments, R⁸ is —OCH₂F, —OCHF₂, —OCF₃, —OCH₂CH₂F, —OCH₂CHF₂, —OCH₂CF₃, —OCHFCH₃, —OCF₂CH₃, —OCHFCH₂F, —OCF₂CH₂F, —OCHFCHF₂, —OCH₂CH₂CH₂F, —OCH₂CH₂CHF₂, —OCH₂CH₂CF₃, —OCH₂CHFCH₂F, —OCH₂CHFCHF₂, —OCH₂CF₂CH₂F, —OCHFCHFCH₂F, —OCF₂CH₂CH₂F, —OCHFCH₂CH₃, —OCH₂CHFCH₃, —OCHFCHFCH₃, —OCHFCF₂CH₃, or —OCF₂CHFCH₃.

In some embodiments, V is CR⁸ and R⁸ is —O(C₁-C₃-alkyl) substituted by —OH. In some embodiments, R⁸ is methoxy, ethoxy, n-propoxy, or isopropoxy, each of which is substituted by 1-4 —OH groups. In some embodiments, R⁸ is —OCH₂OH, —OCH(OH)CH₃, —OCH₂CH₂OH, —OCH(OH)CH₂CH₃, —OCH₂CH(OH)CH₃, —OCH₂CH₂CH₂OH, —OC(CH₃)₂OH, or —OCH(CH₂OH)(CH₃). In some embodiments, R⁸ is —OCH₂CH₂OH.

In some embodiments, V is CR⁸ and R⁸ is —O(C₁-C₃-alkyl) substituted by —R^{8a}, wherein R^{8a} is C₁-C₃ alkyl. In some embodiments, R⁸ is methoxy, ethoxy, n-propoxy, or isopropoxy, each of which is substituted by 1-4 —OR^{8a} groups. In some embodiments, R⁸ is —O(C₁-C₃-alkyl) substituted by —R^{8a}, wherein R^{8a} is —CH₃, —CH₂CH₃, —CH₂CH₂CH₃, or —CH(CH₃)₂. In some embodiments, R⁸ is methoxy substituted by —OR^{8a}. In some embodiments, R⁸ is ethoxy substituted by —OR^{8a}. In some embodiments, R⁸ is n-propoxy substituted by —R^{8a}. In some embodiments, R⁸ is isopropoxy substituted by —OR^{8a}. In certain embodiments, R⁸ is —O(C₁-C₃-alkyl) substituted by —OR^{8a}, wherein R^{8a} is —CH₃. In certain embodiments, R⁸ is —O(C₁-C₃-alkyl) substituted by —OR^{8a}, wherein R^{8a} is —CH₂CH₃. In certain embodiments, R⁸ is —O(C₁-C₃-alkyl) substituted by —OR^{8a}, wherein R^{8a} is —CH₂CH₂CH₃. In certain embodiments, R⁸ is —O(C₁-C₃-alkyl) substituted by —OR^{8a}, wherein R^{8a} is —CH(CH₃)₂. In some embodiments, R⁸ is —OCH₂OCH₃, —OCH₂OCH₂CH₃, —OCH₂OCH₂CH₂CH₃, —OCH₂OCH(CH₃)₂, —OCH(OCH₃)CH₃, —OCH(OCH₂CH₃)CH₃, —OCH(OCH₂CH₂CH₃)CH₃, —OCH(CH₃)OCH(CH₃)₂, —OCH₂CH₂OCH₃, —OCH₂CH₂OCH₂CH₃, —OCH₂CH₂OCH₂CH₂CH₃, —OCH₂CH₂OCH(CH₃)₂, —OCH(OCH₃)CH₂CH₃, —OCH(CH₂CH₃)OCH₂CH₃, —OCH(OCH₂CH₂CH₃)CH₂CH₃, —OCH(OCH(CH₃)₂)CH₂CH₃, —OCH₂CH(CH₃)OCH₃, —OCH₂CH(CH₃)OCH₂CH₃, —OCH₂CH(CH₃)OCH₂CH₂CH₃, —OCH₂CH(CH₃)OCH(CH₃)₂, —OCH₂CH₂CH₂OCH₃, —OCH₂CH₂CH₂OCH₂CH₃, —OCH₂CH₂CH₂OCH₂CH₂CH₃, —OCH₂CH₂CH₂OCH(CH₃)₂, —OC(OCH₃)(CH₃)₂, —OC(OCH₂CH₃)(CH₃)₂, —OC(OCH₂CH₂CH₃)(CH₃)₂, —OC(OCH(CH₃)₂)(CH₃)₂, —OCH(CH₃)CH₂OCH₃, —OCH(CH₃)CH₂OCH₂CH₃, —OCH(CH₃)CH₂OCH₂CH₂CH₃, or —OCH(CH₃)CH₂OCH(CH₃)₂.

In some embodiments, V is C R⁸ and R⁸ is —O(C₁-C₃-alkyl) substituted by 1-4 —NR^{8a}R^{8b} groups. In some embodiments, R⁸ is —O(C₁-C₃-alkyl) substituted by —NR^{8a}R^{8b}, wherein each R^{8a} and R^{8b} are independently H or C₁-C₃ alkyl. In some embodiments, R⁸ is methoxy, ethoxy, n-propoxy, or isopropoxy, each of which is substituted by —NR^{8a}R^{8b}, wherein each R^{8a} and R^{8b} are independently H or C₁-C₃ alkyl. In some embodiments, R⁸ is methoxy, ethoxy, n-propoxy, or isopropoxy, each of which is substituted by —NH₂, —NH(C₁-C₃ alkyl), or —N(C₁-C₃ alkyl)(C₁-C₃ alkyl). In some embodiments, R⁸ is methoxy, ethoxy, n-propoxy, or isopropoxy, each of which is substituted by —NH₂, —NH(methyl), —NH(ethyl), —NH(n-propyl), —NH(isopropyl), —N(methyl)(methyl), —N(methyl)(ethyl), —N(methyl)(n-propyl), —N(methyl)(isopropyl), —N(ethyl)(ethyl), —N(ethyl)(n-propyl), —N(ethyl)(isopropyl), —N(n-propyl)(n-propyl), —N(n-propyl)(isopropyl), or —N(isopropyl)(isopropyl). In some embodiments, R⁸ is methoxy substituted by —NH₂, —NH(methyl), —NH(ethyl), —NH(n-propyl), —NH(isopropyl), —N(methyl)(methyl), —N(methyl)(ethyl), —N(methyl)(n-propyl), —N(methyl)(isopropyl), —N(ethyl)(ethyl), —N(ethyl)(n-propyl), —N(ethyl)(isopropyl), —N(n-propyl)(n-propyl), —N(n-propyl)(isopropyl), or —N(isopropyl)(isopropyl). In some embodiments, R⁸ is methoxy, ethoxy, n-propoxy, or isopropoxy, each of which is substituted by —N(methyl)(methyl). In certain embodiments, R⁸ is —O(C₁-C₃-alkyl) substituted by —NR^{8a}R^{8b}, wherein each R^{8a} and R^{8b} are —CH₃. In some embodiments, R⁸ is methoxy substituted by —N(methyl)(methyl). In some embodiments, R⁸ is ethoxy or n-propoxy substituted by —N(C₁-C₃ alkyl)(C₁-C₃ alkyl). In some embodiments, R⁸ is ethoxy or n-propoxy substituted by —N(methyl)(methyl). In some embodiments, R⁸ is —OCH₂CH₂N(CH₃)₂, or —OCH₂CH₂CH₂N(CH₃)₂. In some embodiments, R⁸ is ethoxy substituted by —NH(C₁-C₃ alkyl). In some embodiments, R⁸ is ethoxy substituted by —NH(methyl). In some embodiments, R⁸ is —OCH₂CH₂NH(CH₃).

In other embodiments, V is C R⁸ and R⁸ is —O(C₁-C₃-alkyl) substituted by —NR^{8a}R^{8b}, wherein each pair of geminal R^{8a} and R^{8b} may be taken together with the nitrogen atom to which they are attached to form an N-heterocycloalkyl wherein the N-heterocycloalkyl is optionally substituted by C₁-C₃ alkyl. In some embodiments, R⁸ is methoxy, ethoxy, n-propoxy, or isopropoxy, each of which is substituted by —NR^{8a}R^{8b}, wherein each pair of geminal R^{8a} and R^{8b} may be taken together with the nitrogen atom to which they are attached to form an N-heterocycloalkyl wherein the N-heterocycloalkyl is optionally substituted by C₁-C₃ alkyl. In some embodiments, each pair of geminal R^{8a} and R^{8b} may be taken together to form a 3- to 7-membered N-heterocycloalkyl wherein the 3- to 7-membered N-heterocycloalkyl is optionally substituted by C₁-C₃ alkyl. In some embodiments, R⁸ is methoxy, ethoxy, n-propoxy, or isopropoxy, each of which is substituted by 3- to 7-membered nitrogen-linked N-heterocycloalkyl wherein the 3- to 7-membered nitrogen-linked N-heterocycloalkyl is optionally substituted by C₁-C₃ alkyl. In some embodiments, R⁸ is methoxy, ethoxy, n-propoxy, or isopropoxy, each of which is substituted by 4- to 6-membered nitrogen-linked N-heterocycloalkyl wherein the 4- to 6-membered nitrogen-linked N-heterocycloalkyl is optionally substituted by C₁-C₃ alkyl. In some embodiments, R⁸ is methoxy, ethoxy, n-propoxy, or isopropoxy, each of which is substituted by 3- to 7-membered nitrogen-linked N-heterocycloalkyl containing 1 nitrogen atom wherein the 3- to 7-membered nitrogen-linked N-heterocycloalkyl is optionally substituted by C₁-C₃ alkyl. In some embodiments, R⁸ is methoxy, ethoxy, n-propoxy, or isopropoxy, each of which is substituted by 3- to 7-membered nitrogen-linked N-heterocycloalkyl containing 2 nitrogen atoms wherein the 3- to 7-membered nitrogen-linked N-heterocycloalkyl is optionally substituted by C₁-C₃ alkyl. In some embodiments, R⁸ is methoxy, ethoxy, n-propoxy, or isopropoxy, each of which is substituted by 4- to 6-membered nitrogen-linked heterocycloalkyl containing 1 nitrogen atom wherein the 4- to 6-membered nitrogen-linked heterocycloalkyl is optionally substituted by C₁-C₃ alkyl. In some embodiments, R⁸ is methoxy, ethoxy, n-propoxy, or isopropoxy, each of which is substituted by 4- to 6-membered nitrogen-linked heterocycloalkyl containing 2 nitrogen atoms wherein the 4- to 6-membered nitrogen-linked heterocycloalkyl is optionally substituted by C₁-C₃ alkyl. In some embodiments, R⁸ is methoxy, ethoxy, n-propoxy, or isopropoxy, each of which is substituted by nitrogen-linked azetidinyl, diazetidinyl, pyrrolidinyl, pyrazolidinyl, imidazolidinyl, piperidinyl, or piperazinyl. In some embodiments, R⁸ is methoxy, ethoxy, n-propoxy, or isopropoxy, each of which is substituted by nitrogen-linked azetidinyl, pyrrolidinyl, or piperidinyl. In some embodiments, R⁸ is methoxy, ethoxy, n-propoxy, or isopropoxy, each of which is substituted by nitrogen-linked azetidinyl, diazetidinyl, pyrrolidinyl, pyrazolidinyl, imidazolidinyl, piperidinyl, or piperazinyl wherein the nitrogen-linked azetidinyl, diazetidinyl, pyrrolidinyl, pyrazolidinyl, imidazolidinyl, piperidinyl, and piperazinyl are substituted by C₁-C₃ alkyl. In some embodiments, R⁸ is methoxy, ethoxy, n-propoxy, or isopropoxy, each of which is substituted by nitrogen-linked azetidinyl, pyrrolidinyl, or piperidinyl wherein the nitrogen-linked azetidinyl, pyrrolidinyl, and piperidinyl are substituted by C₁-C₃ alkyl. In some embodiments, R⁸ is methoxy, ethoxy, n-propoxy, or isopropoxy, each of which is substituted by nitrogen-linked azetidinyl, pyrrolidinyl, or piperidinyl wherein the nitrogen-linked azetidinyl, pyrrolidinyl, and piperidinyl are substituted by CH₃.

In some embodiments, V is C R⁸ and R⁸ is methoxy, ethoxy, n-propoxy, or isopropoxy, each of which is substituted by 4- to 6-membered nitrogen-linked N-heterocycloalkyl containing 1-2 additional ring heteroatoms selected from the group consisting of N and O wherein the 4- to 6-membered nitrogen-linked N-heterocycloalkyl is optionally substituted by C₁-C₃ alkyl. In some embodiments, R⁸ is methoxy, ethoxy, n-propoxy, or isopropoxy, each of which is substituted by 4- to 6-membered nitrogen-linked N-heterocycloalkyl containing 1 oxygen atom wherein the 4- to 6-membered nitrogen-linked N-heterocycloalkyl is optionally substituted by C₁-C₃ alkyl. In some embodiments, R⁸ is methyl, ethyl, n-propyl, or isopropyl, each of which is substituted by 4- to 6-membered nitrogen-linked N-heterocycloalkyl containing 2 nitrogen atoms wherein the 4- to 6-membered nitrogen-linked N-heterocycloalkyl is optionally substituted by C₁-C₃ alkyl. In some embodiments, R⁸ is methoxy, ethoxy, n-propoxy, or isopropoxy, each of which is substituted by diazetidinyl, pyrazolidinyl, imidazolidinyl, piperazinyl, oxetanyl, or morpholinyl. In some embodiments, R⁸ is methoxy, ethoxy, n-propoxy, or isopropoxy, each of which is substituted by diazetidinyl, pyrazolidinyl, imidazolidinyl, piperazinyl, oxetanyl, or morpholinyl wherein the diazetidinyl, pyrazolidinyl, imidazolidinyl, piperazinyl, oxetanyl, or morpholinyl are substituted by C₁-C₃ alkyl. In some embodiments, R⁸ is methoxy, ethoxy, n-propoxy, or isopropoxy, each of which is substituted by diazetidinyl, pyrazolidinyl, imidazolidinyl, piperazinyl, oxetanyl, or morpholinyl wherein the diazetidinyl, pyrazolidinyl, imidazolidinyl, piperazinyl, oxetanyl, or morpholinyl are substituted by CH₃.

In other embodiments, V is C R⁸ and R⁸ is H. In other embodiments, R⁸ is —O(C₂-C₃ alkyl), wherein the C₂-C₃ alkyl is substituted by 1-2 substituents selected from the group consisting of —OH and —NR^{8a}R^{8b}, wherein each R^{8a} and R^{8b} are independently H, or C₁-C₃ alkyl. In other embodiments, R⁸ is —OCH₂CH₂OH, —OCH₂CH₂N(CH₃)₂, or —OCH₂CH₂CH₂N(CH₃)₂.

In some embodiments, W is N. In other embodiments, W is C—CN. In yet other embodiments, V is C R⁸ and W is N. In still other embodiments, V is CR⁸ and W is C—CN. In still yet other embodiments, V is N and W is N. In some embodiments, V is N and W is C—CN.

In some embodiments, G is O and ring A is

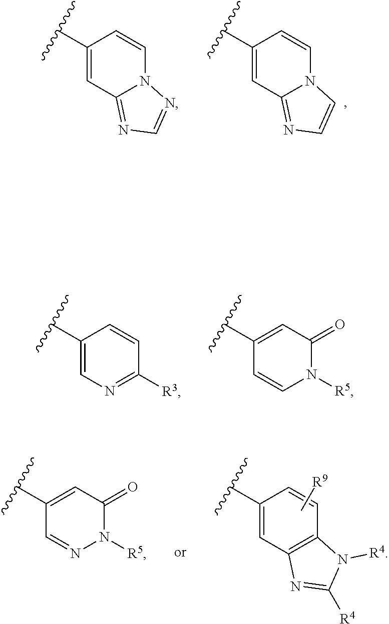

In some embodiments, G is O and ring A is

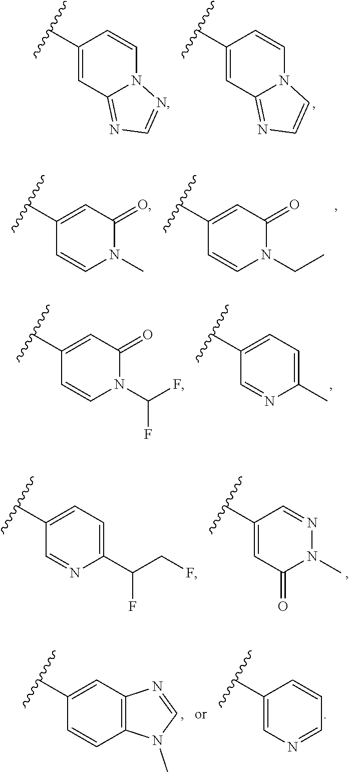

In some embodiments, G is O; ring A is

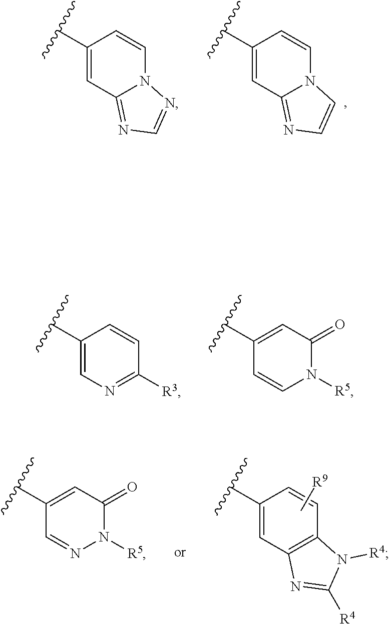

and R¹ is C₁-C₃ alkyl optionally substituted by 1-3 substituents selected from the group consisting of F, —OH, —N(C₁-C₃ alkyl)(C₁-C₃ alkyl), and 4- to 6-membered heterocyclyl containing 1-2 ring heteroatoms selected from the group consisting of N and O. In some embodiments, G is O; ring A is

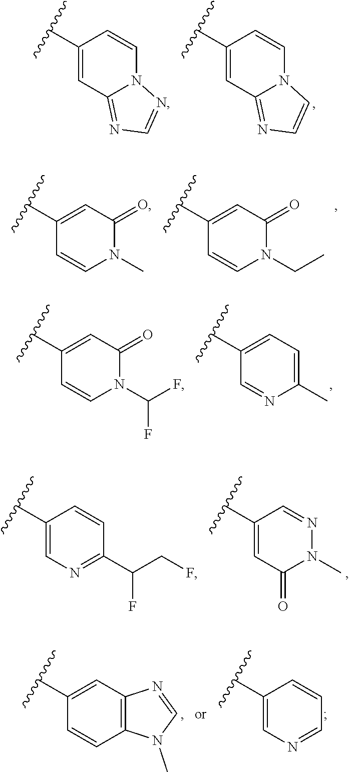

and R¹ is methyl, ethyl, tert-butyl, methyl-OH, or ethyl-OH. In some embodiments, G is O; ring A is

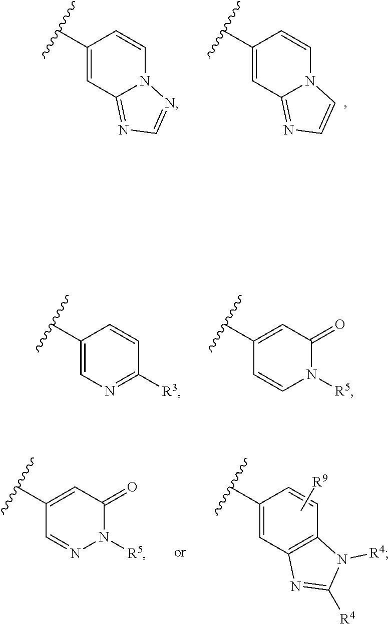

and R¹ is C₃-C₆ cycloalkyl optionally substituted by 1-3 substituents selected from the group consisting of F, —OH, C₁-C₃ alkyl, —N(C₁-C₃ alkyl)(C₁-C₃ alkyl), and 4- to 6-membered heterocyclyl containing 1-2 ring heteroatoms selected from the group consisting of N and O. In some embodiments, G is O; ring A is

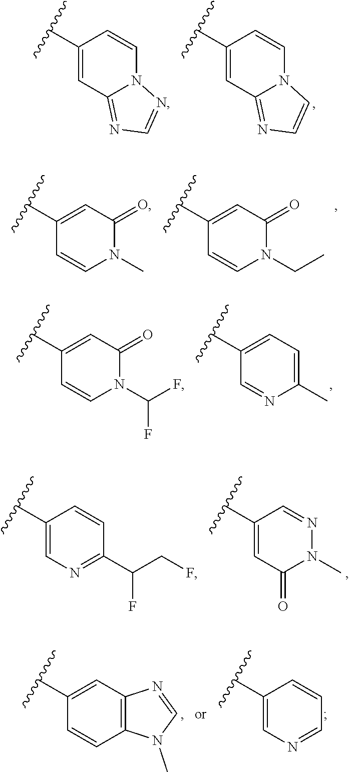

and R¹ is cyclopropyl or cyclopropyl substituted by —CH₃.

In some embodiments, the compound of formula (I) is a compound of formula (I-a):

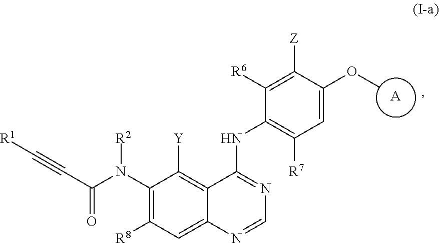

wherein R¹, R², R⁶, R⁷, R⁸, Y, Z, and ring A are as defined for formula (I).

In some embodiments, the compound of formula (I) is a compound of formula (I-b):

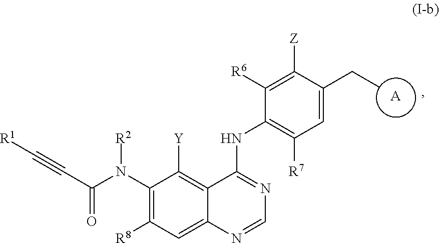

wherein R¹, R², R⁶, R⁷, R⁸, Y, Z, and ring A are as defined for formula (I).

In some embodiments, the compound of formula (I) is a compound of formula (I-c):

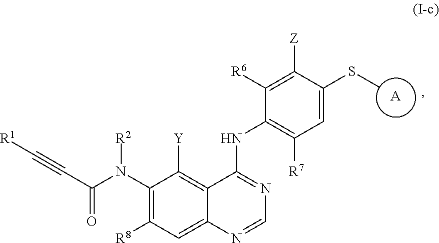

wherein R¹, R², R⁶, R⁷, R⁸, Y, Z, and ring A are as defined for formula (I).

In some embodiments, the compound of formula (I) is a compound of formula (I-d):

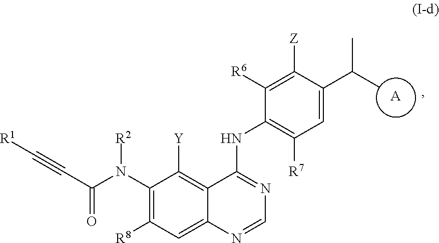

wherein R¹, R², R⁶, R⁷, R⁸, Y, Z, and ring A are as defined for formula (I).

In some embodiments, the compound of formula (I) is a compound of formula (I-e):

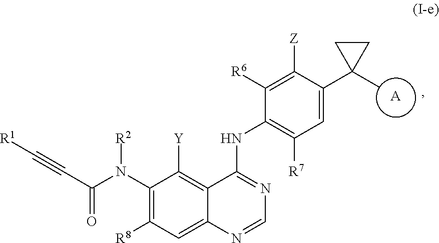

wherein R¹, R², R⁶, R⁷, R⁸, Y, Z, and ring A are as defined for formula (I).

In some embodiments, the compound of formula (I-a) is a compound of formula (I-a-1), (I-a-2), (I-a-3), (I-a-4), (I-a-5), (I-a-6), or (I-a-7):

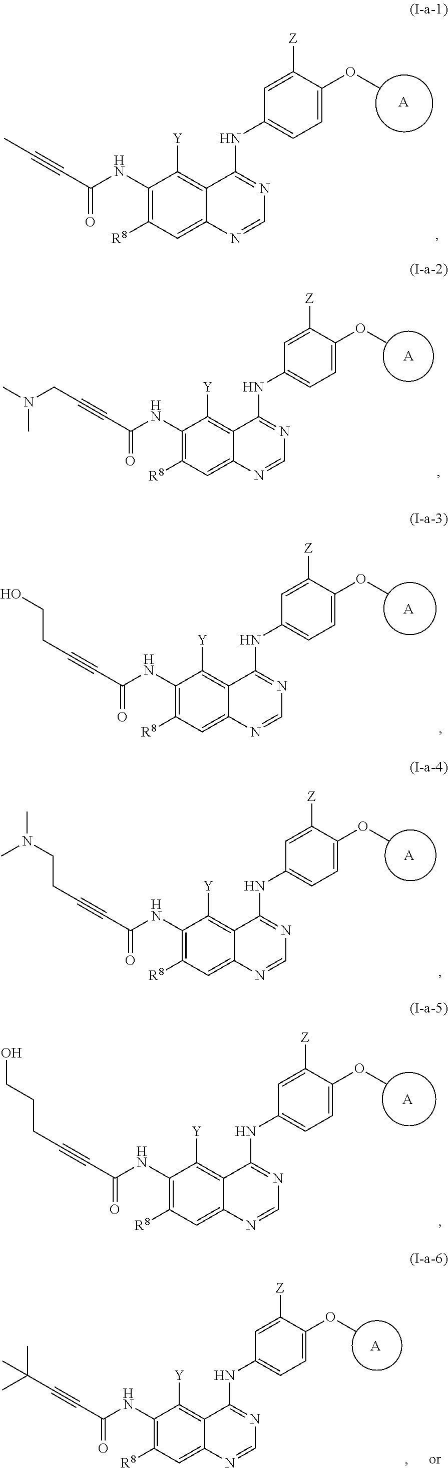

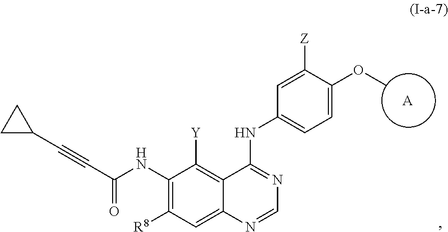

wherein ring A, R⁸, Y and Z are as defined for formula (I). In some embodiments, the compound is a compound of formula (I-a-1). In some embodiments, the compound is a compound of formula (I-a-2). In some embodiments, the compound is a compound of formula (I-a-3). In some embodiments, the compound is a compound of formula (I-a-4). In some embodiments, the compound is a compound of formula (I-a-5). In some embodiments, the compound is a compound of formula (I-a-6). In some embodiments, the compound is a compound of formula (I-a-7). In any variation of formula (I-a-1), (I-a-2), (I-a-3), (I-a-4), (I-a-5), (I-a-6), or (I-a-7), Z is H, F, Cl, or —CH₃, R⁸ is H, —OCH₂CH₂OH, —OCH₂CH₂N(CH₃)₂, or —OCH₂CH₂CH₂N(CH₃)₂, Y is H, F, or —OCH₃, and Ring A is

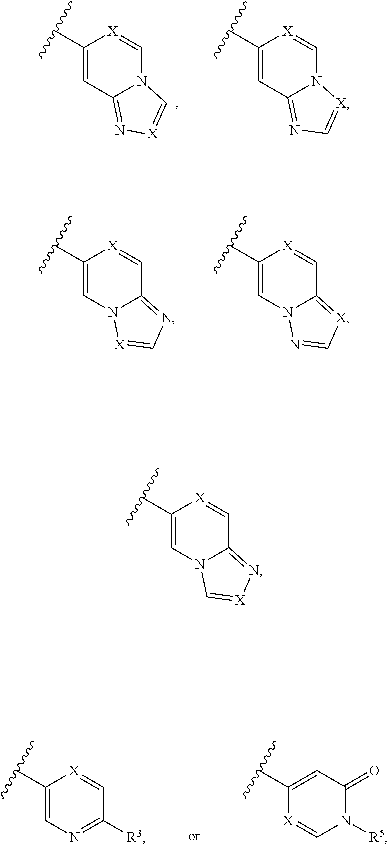

wherein each X is independently N or CH. In some embodiments, Ring A is

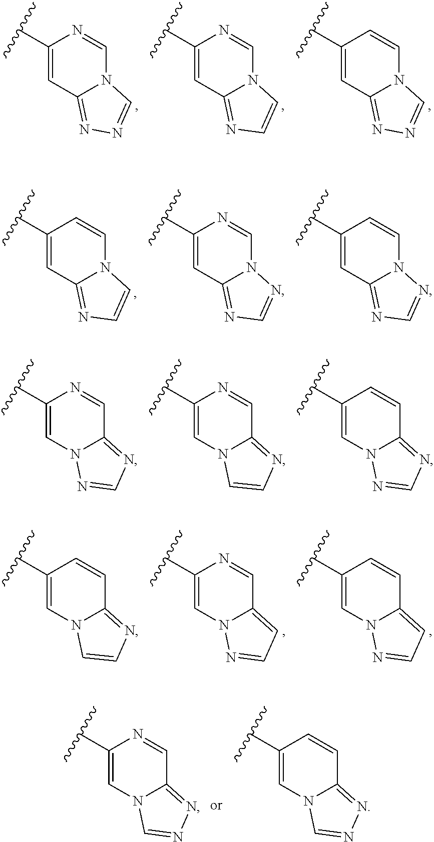

In some embodiments, Ring A is

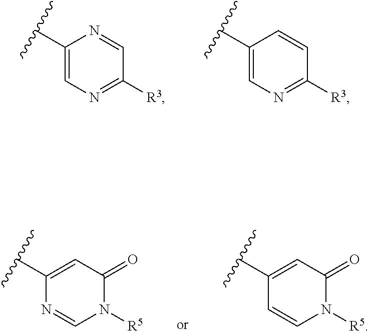

In some embodiments, Ring A is

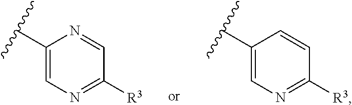

and R³ is cyclopropyl, —CH₃, —CH₂CH₃, —CHF₂, —CF₃, —CHF—CH₂F, or —CD₃. In some embodiments, Ring A is

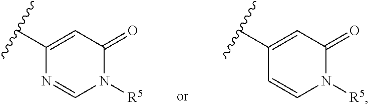

and R⁵ is cyclopropyl, —CH₂-cyclopropyl, —CH₃, —CH₂CH₃, —CHF₂, or —CD₃.

In some embodiments, R⁸ is H. In other embodiments, R⁸ is H, —OCH₂CH₂OH, —OCH₂CH₂N(CH₃)₂, or —OCH₂CH₂CH₂N(CH₃)₂.

In some embodiments, the compound of formula (I-b) is a compound of formula (I-b-3), (I-b-4), (I-b-6), or (I-b-7):

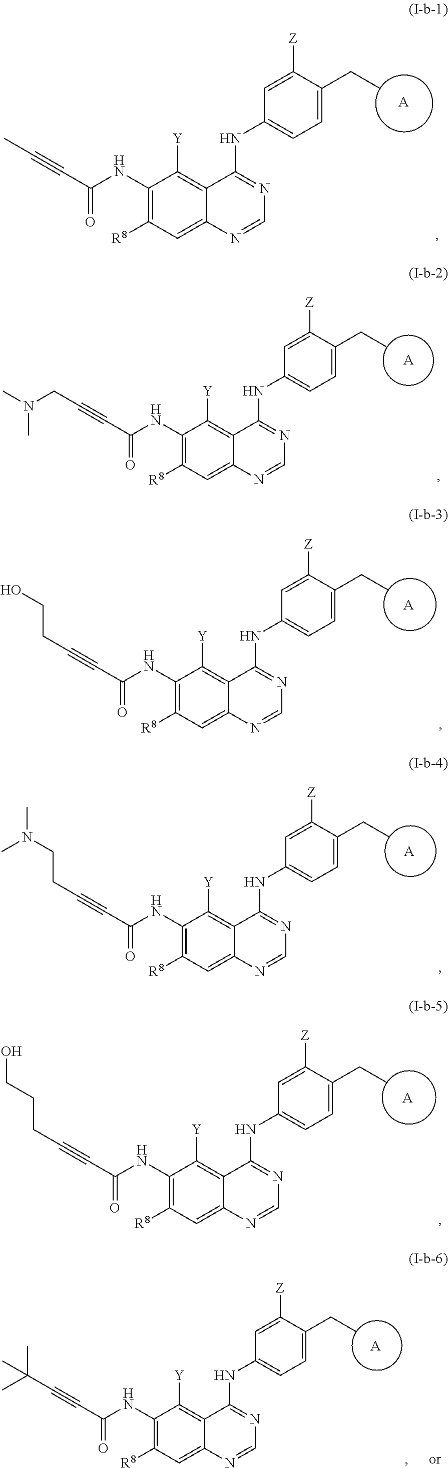

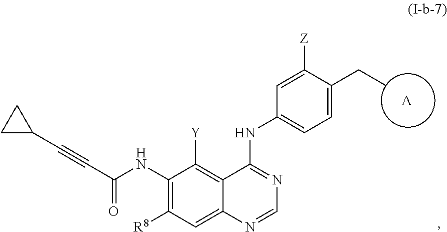

wherein ring A, R⁸, Y and Z are as defined for formula (I). In some embodiments, the compound is a compound of formula (I-b-1). In some embodiments, the compound is a compound of formula (I-b-2). In some embodiments, the compound is a compound of formula (I-b-3). In some embodiments, the compound is a compound of formula (I-b-4). In some embodiments, the compound is a compound of formula (I-b-5). In some embodiments, the compound is a compound of formula (I-b-6). In some embodiments, the compound is a compound of formula (I-b-7). In any variation of formula (I-b-1), (I-b-2), (I-b-3), (I-b-4), (I-b-5), (I-b-6), or (I-b-7), Z is H, F, Cl, or —CH₃, R⁸ is H, —OCH₂CH₂OH, —OCH₂CH₂N(CH₃)₂, or —OCH₂CH₂CH₂N(CH₃)₂, Y is H, F, or —OCH₃, and Ring A is

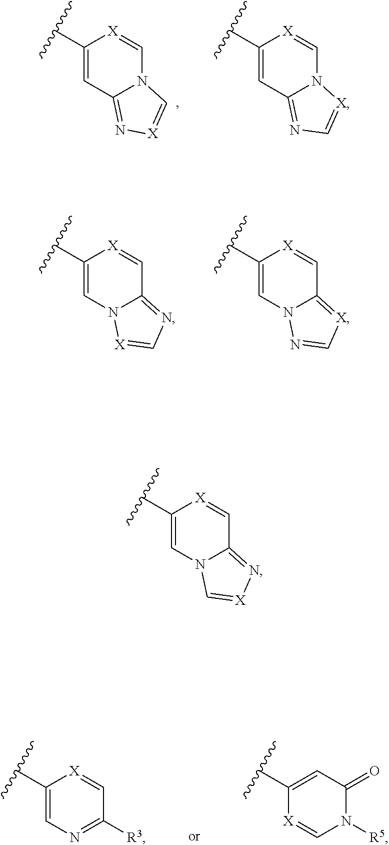

wherein each X is independently N or CH. In some embodiments, Ring A is

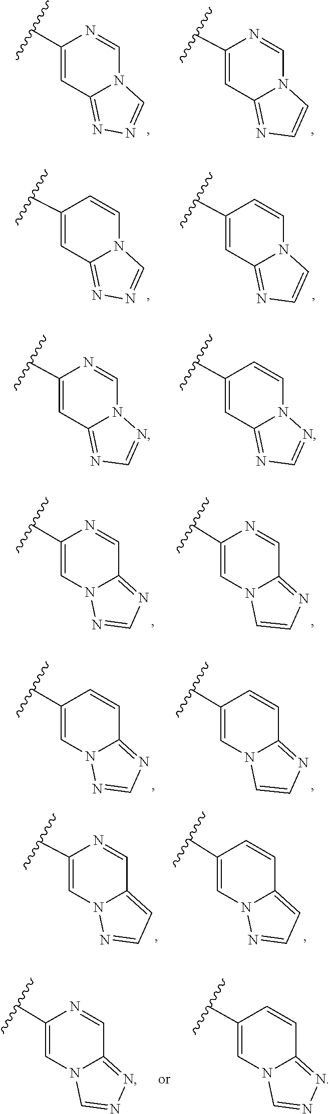

In some embodiments, Ring A is

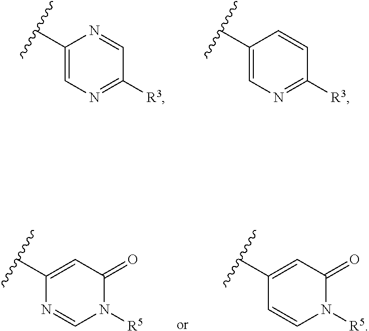

In some embodiments, Ring A is

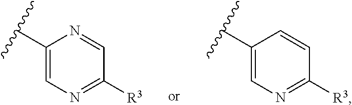

and R³ is cyclopropyl, —CH₃, —CH₂CH₃, —CHF₂, —CHF—CH₂F, —CF₃, or —CD₃. In some embodiments, Ring A is

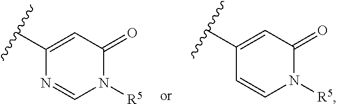

and R⁵ is cyclopropyl, —CH₂-cyclopropyl, —CH₃, —CH₂CH₃, —CHF₂, or —CD₃.

In some embodiments, R⁸ is H. In other embodiments, R⁸ is H, —OCH₂CH₂OH, —OCH₂CH₂N(CH₃)₂, or —OCH₂CH₂CH₂N(CH₃)₂.

In some embodiments, the compound of formula (I-c) is a compound of formula (I-c-1), (I-c-2), (I-c-3), (I-c-4), (I-c-5), (I-c-6), or (I-c-7):

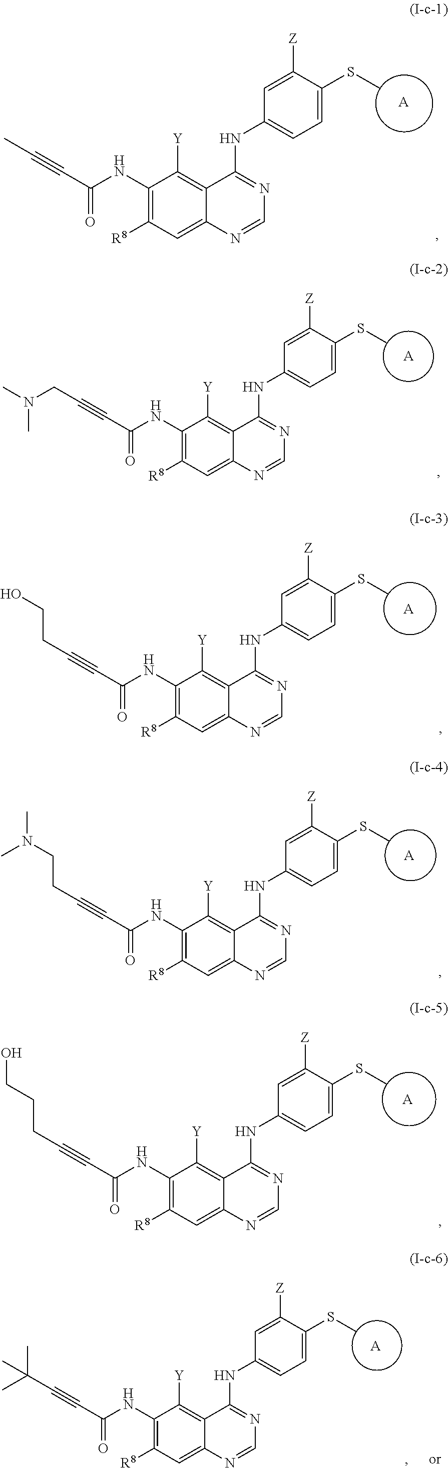

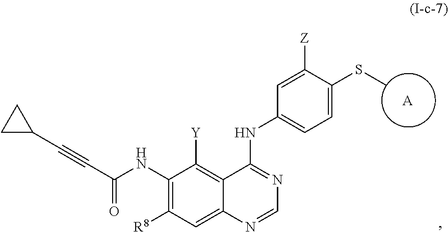

wherein ring A, R⁸, Y and Z are as defined for formula (I)(I). In some embodiments, the compound is a compound of formula (I-c-1). In some embodiments, the compound is a compound of formula (I-c-2). In some embodiments, the compound is a compound of formula (I-c-3). In some embodiments, the compound is a compound of formula (I-c-4). In some embodiments, the compound is a compound of formula (I-c-5). In some embodiments, the compound is a compound of formula (I-c-6). In some embodiments, the compound is a compound of formula (I-c-7). In any variation of formula (I-c-1), (I-c-5), (I-c-6), or (I-c-7), Z is H, F, Cl, or —CH₃, R⁸ is H, —OCH₂CH₂OH, —OCH₂CH₂N(CH₃)₂, or —OCH₂CH₂CH₂N(CH₃)₂, Y is H, F, or —OCH₃, and Ring A is

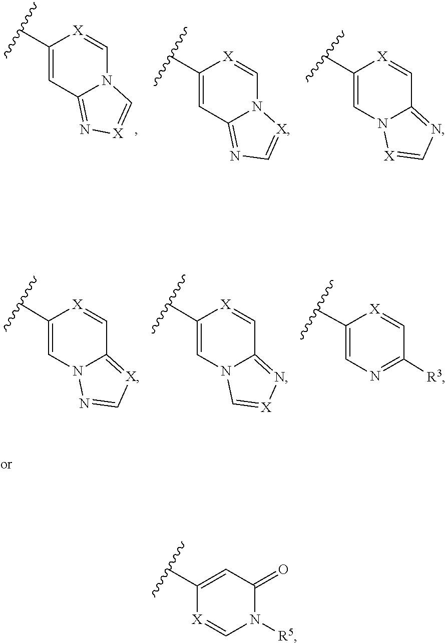

wherein each X is independently N or CH. In some embodiments, Ring A is

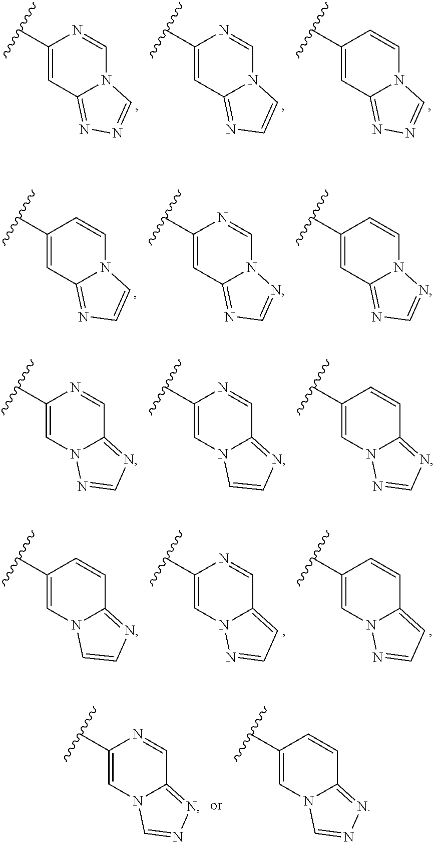

In some embodiments, Ring A is

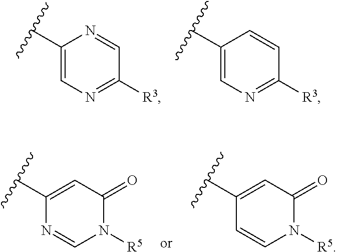

In some embodiments, Ring A is

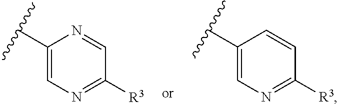

and R³ is cyclopropyl, —CH₃, —CH₂CH₃, —CHF₂, —CF₃, —CHF—CH₂F, or —CD₃. In some embodiments, Ring A is

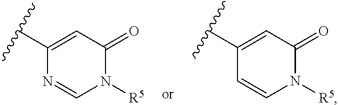

and R⁵ is cyclopropyl, —CH₂-cyclopropyl, —CH₃, —CH₂CH₃, —CHF₂, or —CD₃.

In some embodiments, R⁸ is H. In other embodiments, R⁸ is H, —OCH₂CH₂OH, —OCH₂CH₂N(CH₃)₂, or —OCH₂CH₂CH₂N(CH₃)₂.

In some embodiments, the compound of formula (I-d) is a compound of formula (I-d-1), (I-d-2), (I-d-3), (I-d-4), (I-d-5), (I-d-6), or (I-d-7):

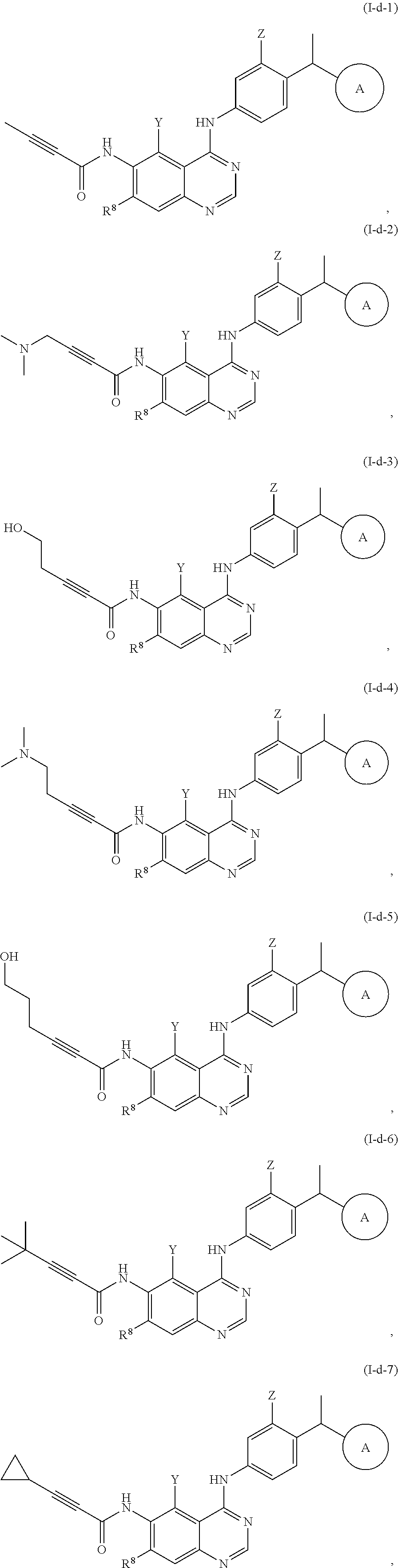

wherein ring A, R⁸, Y and Z are as defined for formula (I). In some embodiments, the compound is a compound of formula (I-d-1). In some embodiments, the compound is a compound of formula (I-d-2). In some embodiments, the compound is a compound of formula (I-d-3). In some embodiments, the compound is a compound of formula (I-d-4). In some embodiments, the compound is a compound of formula (I-d-5). In some embodiments, the compound is a compound of formula (I-d-6). In some embodiments, the compound is a compound of formula (I-d-7). In any variation of formula (I-d-1), (I-d-2), (I-d-3), (I-d-4), (I-d-5), (I-d-6), or (I-d-7), Z is H, F, Cl, or —CH₃, R⁸ is H, —OCH₂CH₂OH, —OCH₂CH₂N(CH₃)₂, or —OCH₂CH₂CH₂N(CH₃)₂, Y is H, F, or —OCH₃, and Ring A is

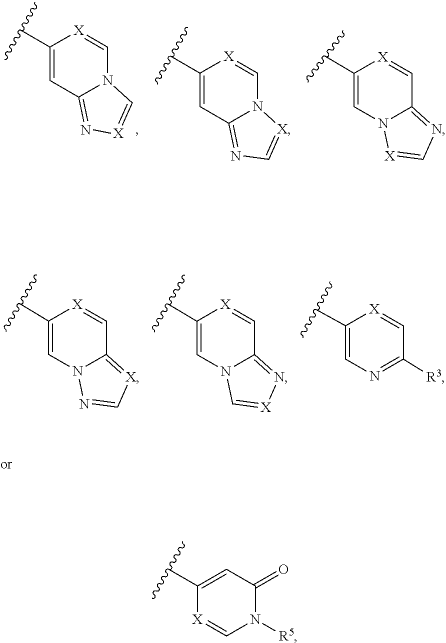

wherein each X is independently N or CH. In some embodiments, Ring A is

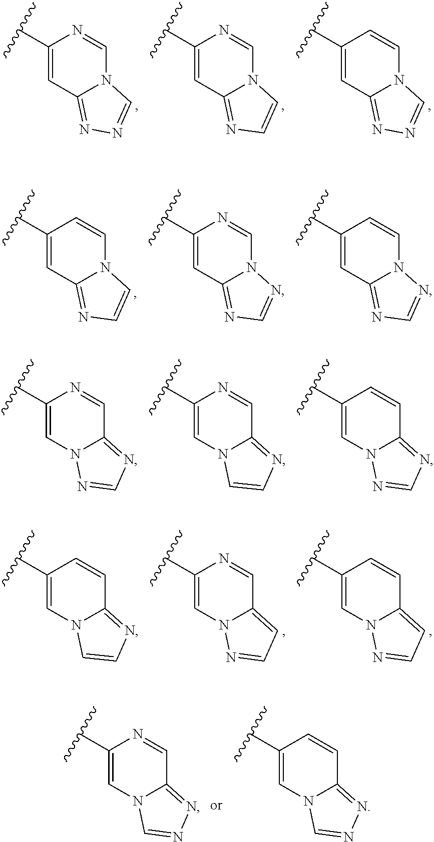

In some embodiments, Ring A is

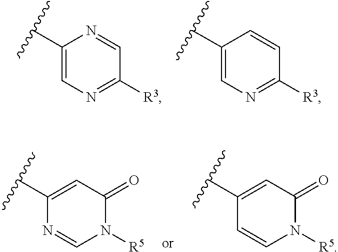

In some embodiments, Ring A is

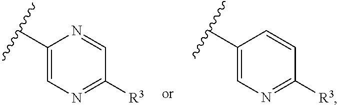

and R³ is cyclopropyl, —CH₃, —CH₂CH₃, —CHF₂, —CF₃, —CHF—CH₂F, or —CD₃. In some embodiments, Ring A is

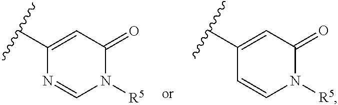

and R⁵ is cyclopropyl, —CH₂-cyclopropyl, —CH₃, —CH₂CH₃, —CHF₂, or —CD₃. In some embodiments, R⁸ is H. In other embodiments, R⁸ is H, —OCH₂CH₂OH, —OCH₂CH₂N(CH₃)₂, or —OCH₂CH₂CH₂N(CH₃)₂.

In some embodiments, the compound of formula (I-e) is a compound of formula (I-e-1), (I-e-2), (I-e-3), (I-e-4), (I-e-5), (I-e-6), or (I-e-7):

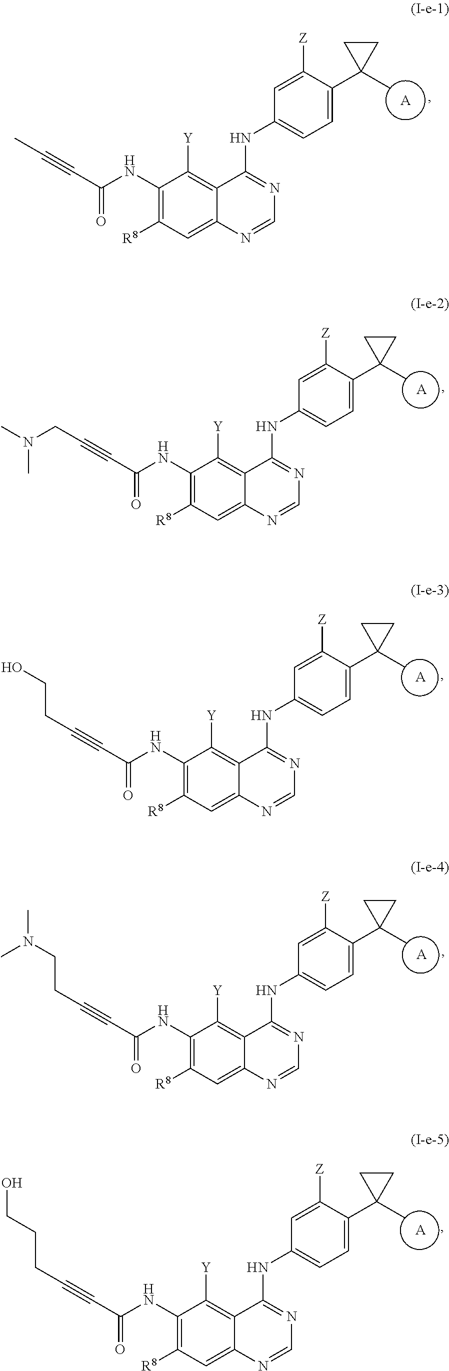

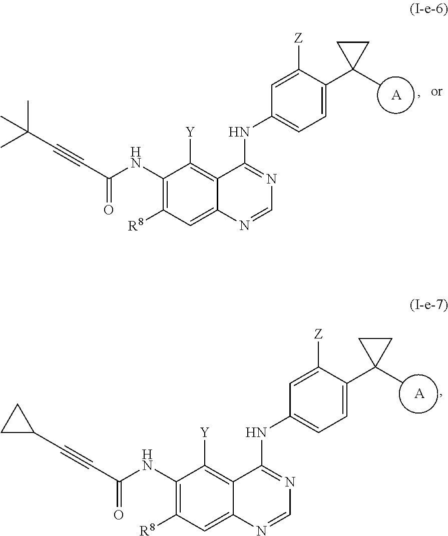

wherein ring A, R⁸, Y and Z are as defined for formula (I). In some embodiments, the compound is a compound of formula (I-e-1). In some embodiments, the compound is a compound of formula (I-e-2). In some embodiments, the compound is a compound of formula (I-e-3). In some embodiments, the compound is a compound of formula (I-e-4). In some embodiments, the compound is a compound of formula (I-e-5). In some embodiments, the compound is a compound of formula (I-e-6). In some embodiments, the compound is a compound of formula (I-e-7). In any variation of formula (I-e-1), (I-e-5) (I-e-6), or (I-e-7), Z is H, F, Cl, or —CH₃, R⁸ is H, —OCH₂CH₂OH, —OCH₂CH₂N(CH₃)₂, or —OCH₂CH₂CH₂N(CH₃)₂, Y is H, F, or —OCH₃, and Ring A is

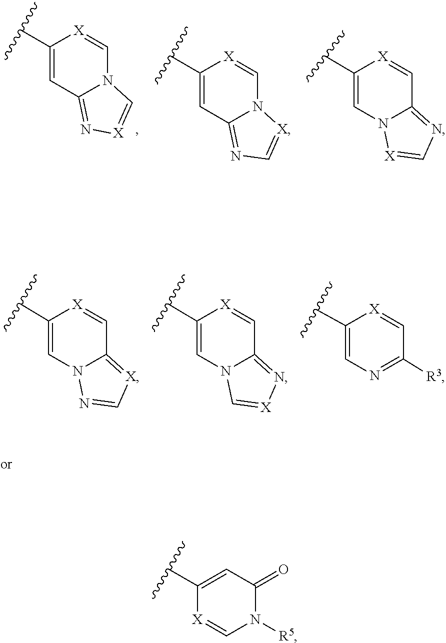

wherein each X is independently N or CH. In some embodiments, Ring A is

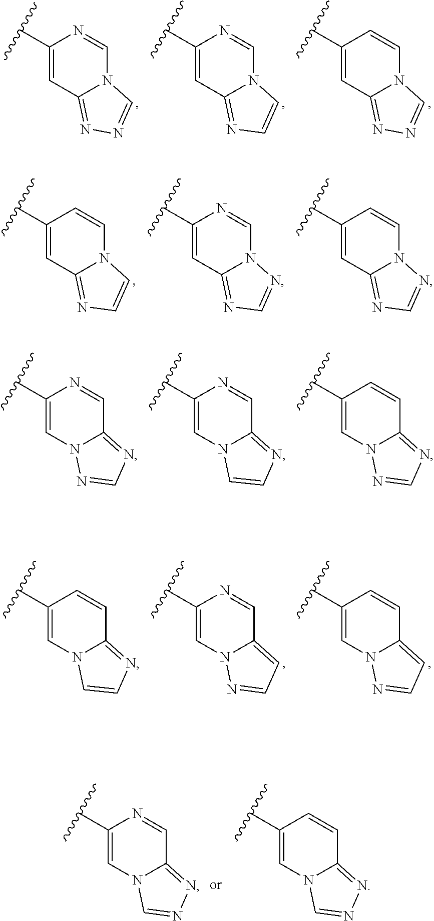

In some embodiments, Ring A is

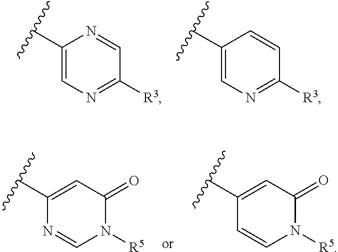

In some embodiments, Ring A is

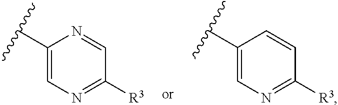

and R³ is cyclopropyl, —CH₃, —CH₂CH₃, —CHF₂, —CF₃, —CHF—CH₂F, or —CD₃. In some embodiments, Ring A is

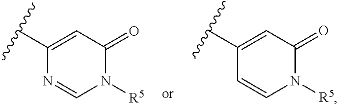

and R⁵ is cyclopropyl, —CH₂-cyclopropyl, —CH₃, —CH₂CH₃, —CHF₂, or —CD₃. In some embodiments, R⁸ is H. In other embodiments, R⁸ is H, —OCH₂CH₂OH, —OCH₂CH₂N(CH₃)₂, or —OCH₂CH₂CH₂N(CH₃)₂.

In some embodiments, the compound of formula (II) is a compound of formula (II-a):

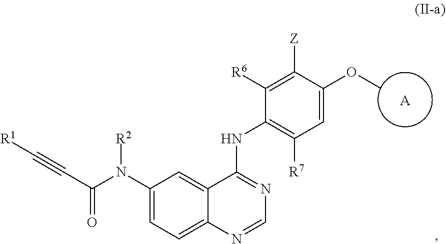

wherein R¹, R², R⁶, R⁷, Z, and ring A are as defined for formula (II).

In some embodiments, the compound of formula (II) is a compound of formula (II-b):

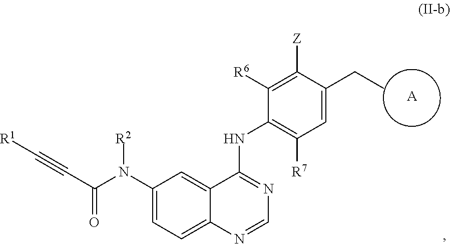

wherein R¹, R², R⁶, R⁷, Z, and ring A are as defined for formula (II).

In some embodiments, the compound of formula (II) is a compound of formula (II-c):

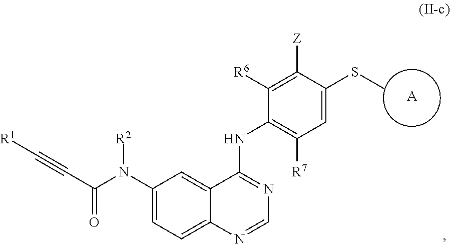

wherein R¹, R², R⁶, R⁷, Z, and ring A are as defined for formula (II).

In some embodiments, the compound of formula (II-a) is a compound of formula (II-a-1), (II-a-2), or (II-a-3):

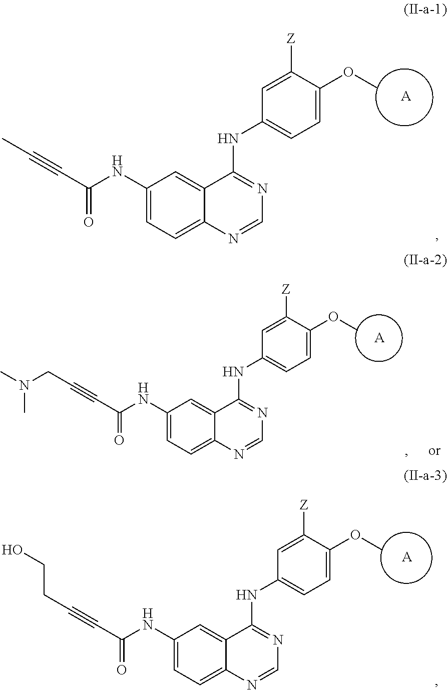

wherein ring A and Z are as defined for formula (II). In some embodiments, the compound is a compound of formula (II-a-1). In some embodiments, the compound is a compound of formula (II-a-2). In some embodiments, the compound is a compound of formula (II-a-3). In any variation of formula (II-a-1), (II-a-2), or (II-a-3), Z is H, F, Cl, or —CH₃, and Ring A is

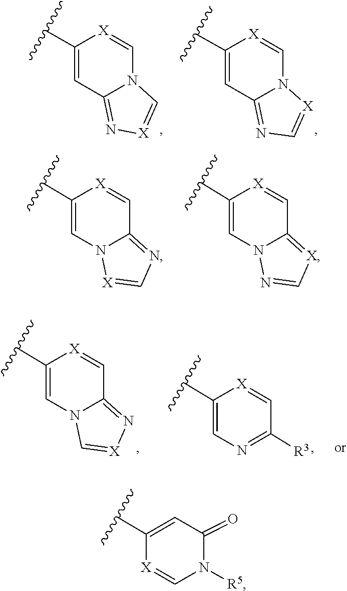

wherein each X is independently N or CH. In some embodiments, Ring A is

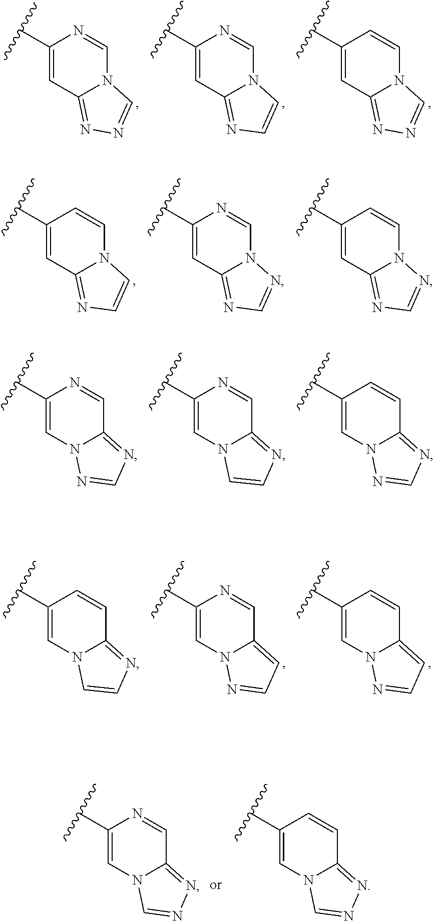

In some embodiments, Ring A is

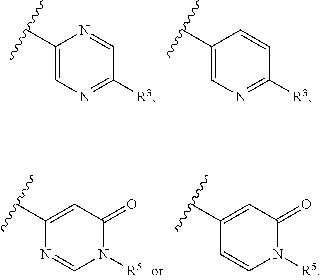

In some embodiments, Ring A is

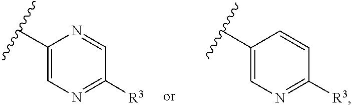

and R³ is —CH₃, —CH₂CH₃, —CHF₂, or —CD₃. In some embodiments, Ring A is

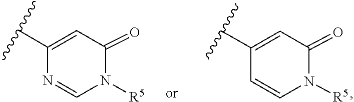

and R⁵ is —CH₃, —CH₂CH₃, —CHF₂, or —CD₃.

In some embodiments, the compound of formula (II-b) is a compound of formula (II-b-1), (II-b-2), or (II-b-3):

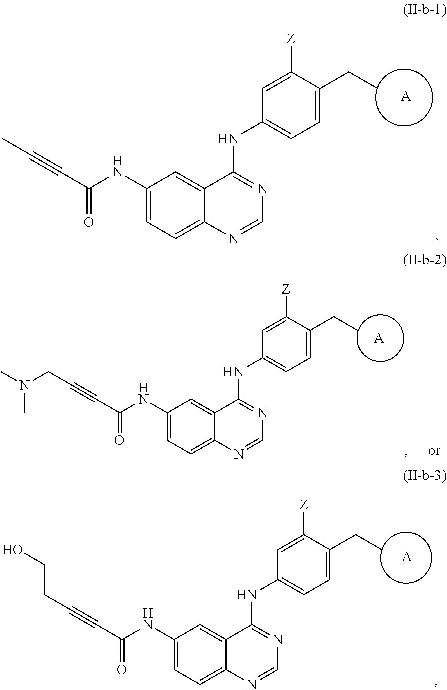

wherein ring A and Z are as defined for formula (II). In some embodiments, the compound is a compound of formula (II-b-1). In some embodiments, the compound is a compound of formula (II-b-2). In some embodiments, the compound is a compound of formula (II-b-3). In any variation of formula (II-b-1), (II-b-2), or (II-b-3), Z is H, F, Cl, or —CH₃, and Ring A is

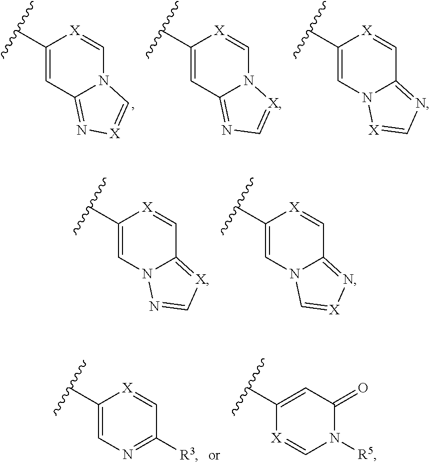

wherein each X is independently N or CH. In some embodiments, Ring A is

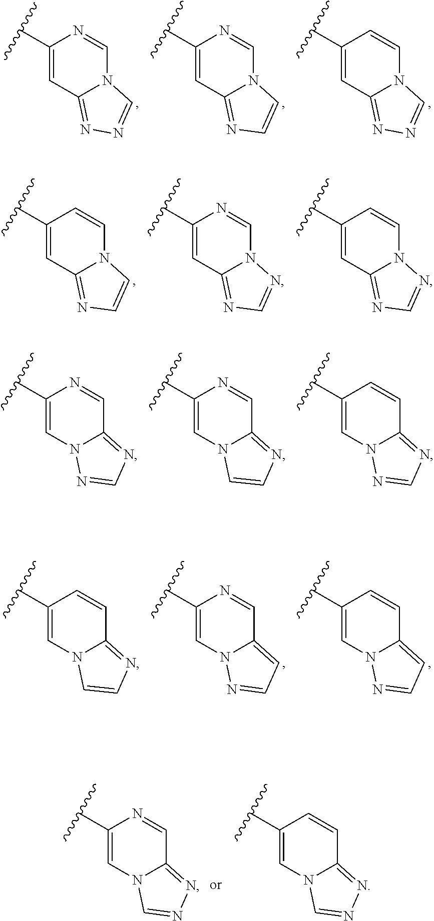

In some embodiments, Ring A is

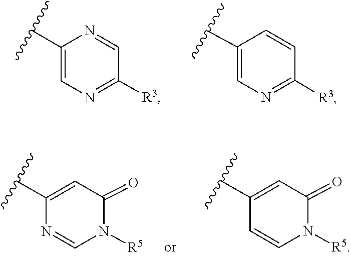

In some embodiments, Ring A is

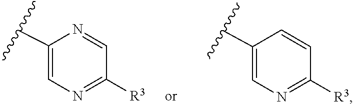

and R³ is —CH₃, —CH₂CH₃, —CHF₂, or —CD₃. In some embodiments, Ring A is

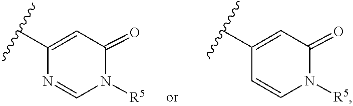

and R⁵ is —CH₃, —CH₂CH₃, —CHF₂, or —CD₃.

In some embodiments, the compound of formula (II-c) is a compound of formula (II-c-1), (II-c-2), or (II-c-3):

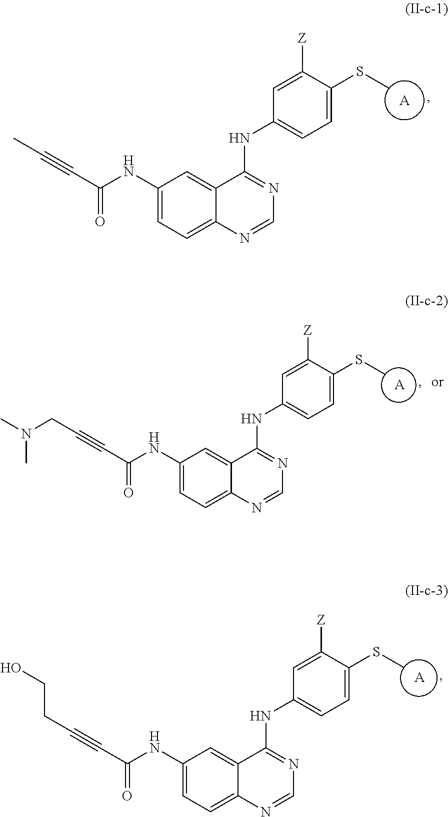

wherein ring A and Z are as defined for formula (II). In some embodiments, the compound is a compound of formula (II-c-1). In some embodiments, the compound is a compound of formula (II-c-2). In some embodiments, the compound is a compound of formula (II-c-3). In any variation of formula (II-c-1), (II-c-2), or (II-c-3), Z is H, F, Cl, or —CH₃, and Ring A is

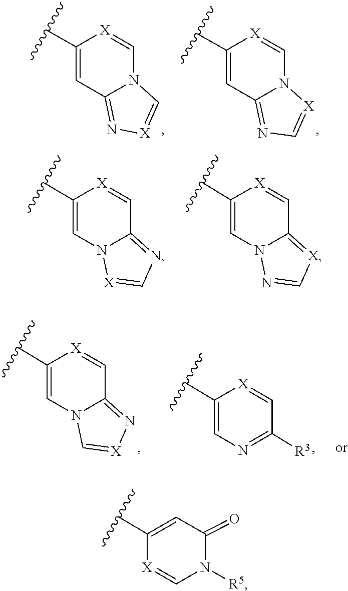

wherein each X is independently N or CH. In some embodiments, Ring A is

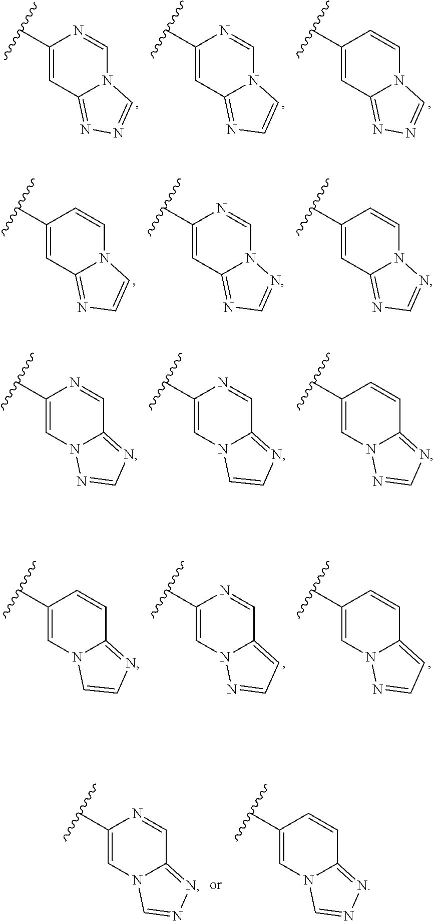

In some embodiments, Ring A is

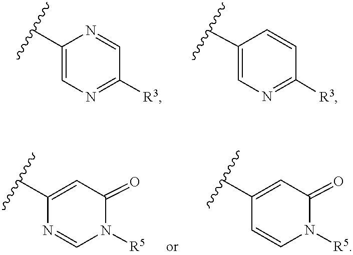

In some embodiments, Ring A is

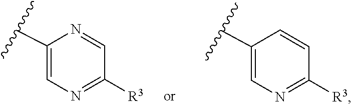

and R³ is —CH₃, —CH₂CH₃, —CHF₂, or —CD₃. In some embodiments, Ring A is

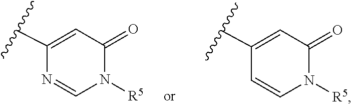

and R⁵ is —CH₃, —CH₂CH₃, —CHF₂, or —CD₃.

In some embodiments, the compound of formula (II-a) is a compound of formula (II-d-1), (II-d-2), (II-d-3), (II-d-4), (II-d-5), (II-d-6), (II-d-7), (II-d-8), (II-d-9), (II-d-10), (II-d-11), (II-d-12), (II-d-13), (II-d-14), (II-d-15), (II-d-16), (II-d-17), or (II-d-18):

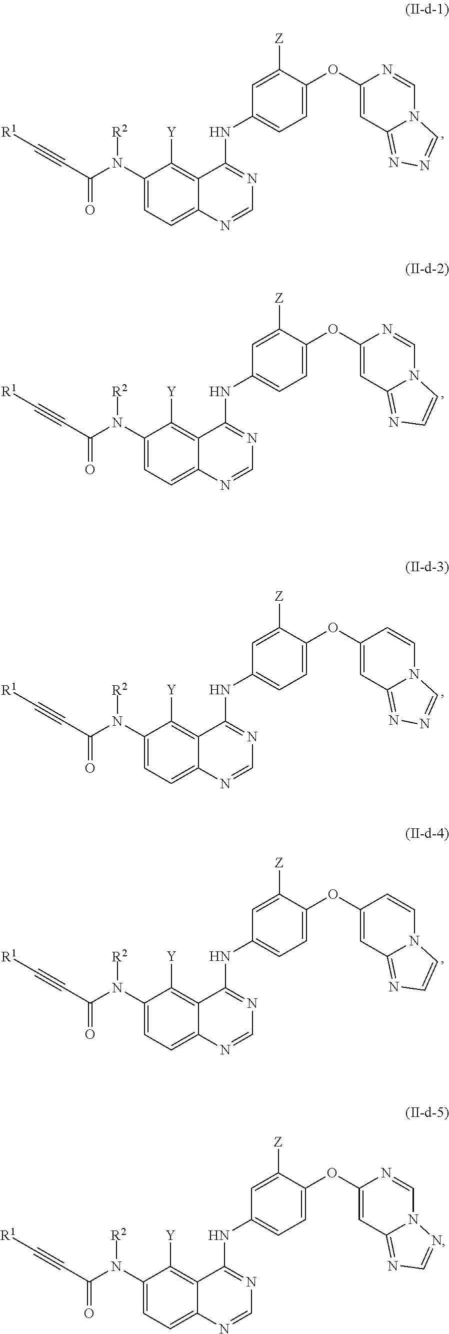

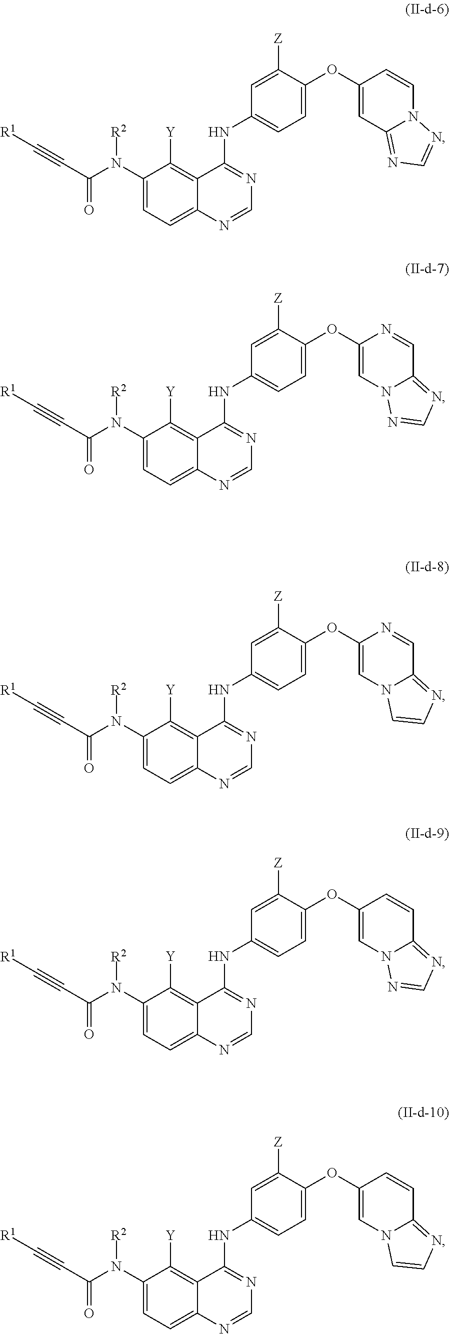

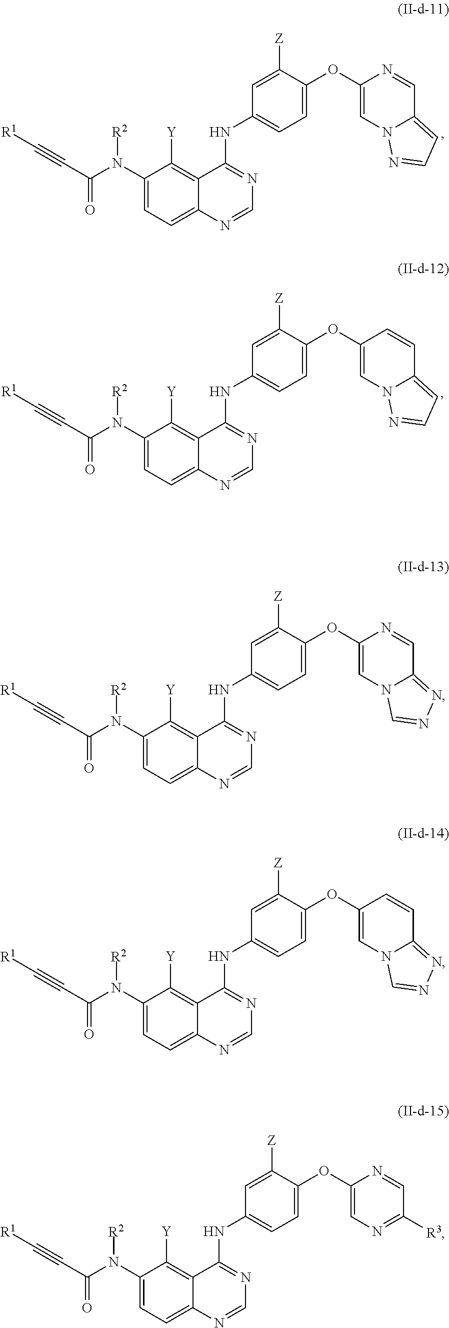

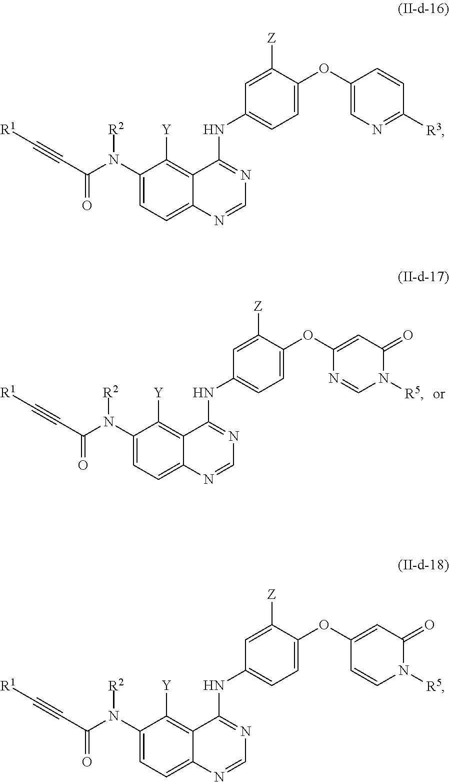

wherein R¹, R², R³, R⁵, Y, and Z are as defined for formula (II). In some embodiments, the compound is a compound of formula (II-d-1). In some embodiments, the compound is a compound of formula (II-d-2). In some embodiments, the compound is a compound of formula (II-d-3). In some embodiments, the compound is a compound of formula (II-d-4). In some embodiments, the compound is a compound of formula (II-d-5). In some embodiments, the compound is a compound of formula (II-d-6). In some embodiments, the compound is a compound of formula (II-d-7). In some embodiments, the compound is a compound of formula (II-d-8). In some embodiments, the compound is a compound of formula (II-d-9). In some embodiments, the compound is a compound of formula (II-d-10). In some embodiments, the compound is a compound of formula (II-d-11). In some embodiments, the compound is a compound of formula (II-d-12). In some embodiments, the compound is a compound of formula (II-d-13). In some embodiments, the compound is a compound of formula (II-d-14). In some embodiments, the compound is a compound of formula (II-d-15). In some embodiments, the compound is a compound of formula (II-d-16). In some embodiments, the compound is a compound of formula (II-d-17). In some embodiments, the compound is a compound of formula (II-d-18). In some embodiments, R¹ is C₁-C₂ alkyl optionally substituted by —OH or —N(C₁-C₃ alkyl)(C₁-C₃ alkyl). In some embodiments, R¹ is unsubstituted C₁-C₂ alkyl. In some embodiments, R¹ is —CH₃. In some embodiments, R¹ is C₁-C₂ alkyl substituted by —N(C₁-C₂ alkyl)(C₁-C₂ alkyl). In some embodiments, R¹ is —CH₂N(CH₃)₂. In some embodiments, R¹ is C₁-C₂ alkyl substituted by —OH. In some embodiments, R¹ is —CH₂CH₂OH. In some embodiments, R² is H, —CH₃, or —CH₂CH₃. In some embodiments, R² is H. In some embodiments, R² is —CH₃. In some embodiments, Y is H, F, or —OCH₃. In some embodiments, Y is H. In some embodiments of the compound of formula (II-d-15) or (II-d-16), R³ is —CH₃, —CH₂CH₃, —CHF₂, or —CD₃. In some embodiments of the compound of formula (II-d-17) or (II-d-18), R⁵ is —CH₃, —CH₂CH₃, —CHF₂, or —CD₃.

In some embodiments, the compound of formula (II-b) is a compound of formula (II-e-1), (II-e-2), (II-e-3), (II-e-4), (II-e-5), (II-e-6), (II-e-7), (II-e-8), (II-e-9), (II-e-10), (II-e-11), (II-e-12), (II-e-13), (II-e-14), (II-e-15), (II-e-16), (II-e-17), or (II-e-18):

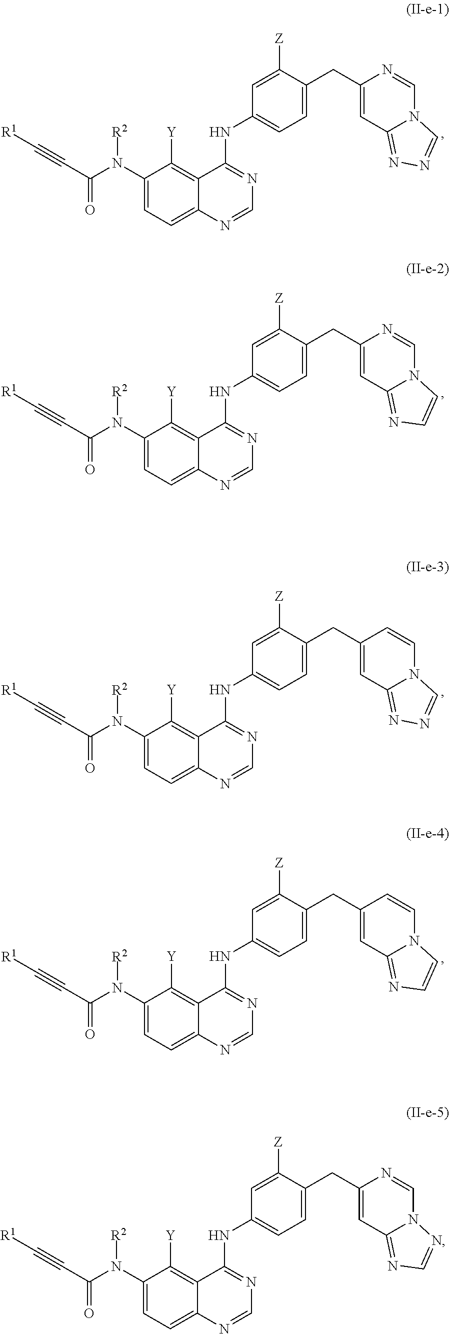

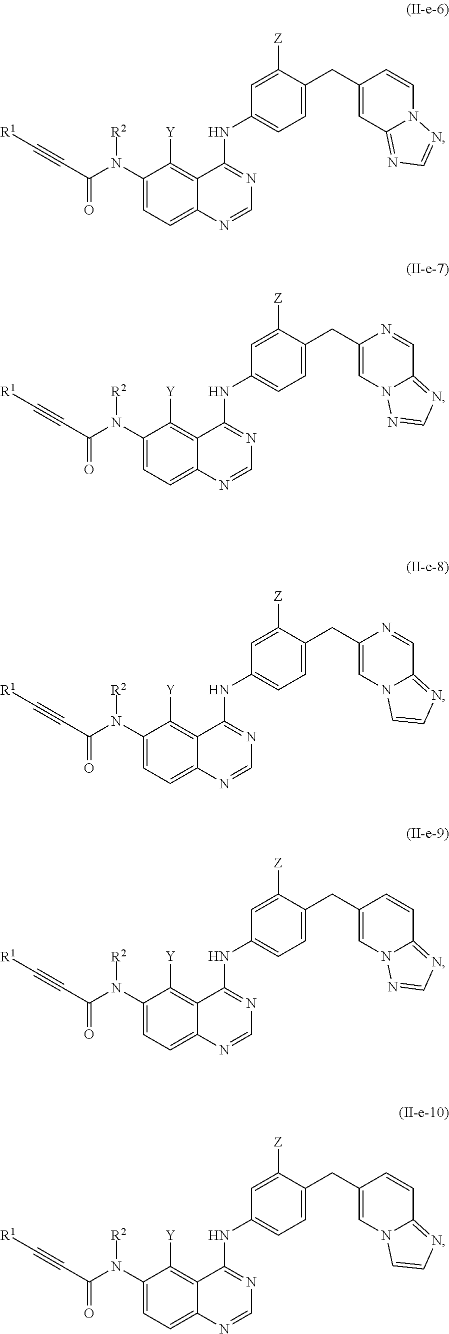

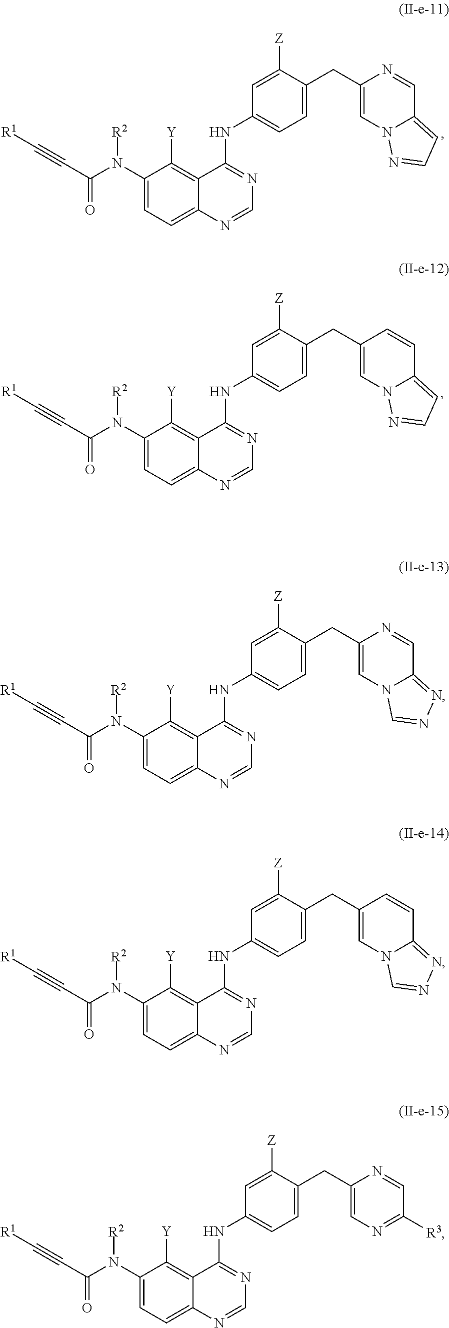

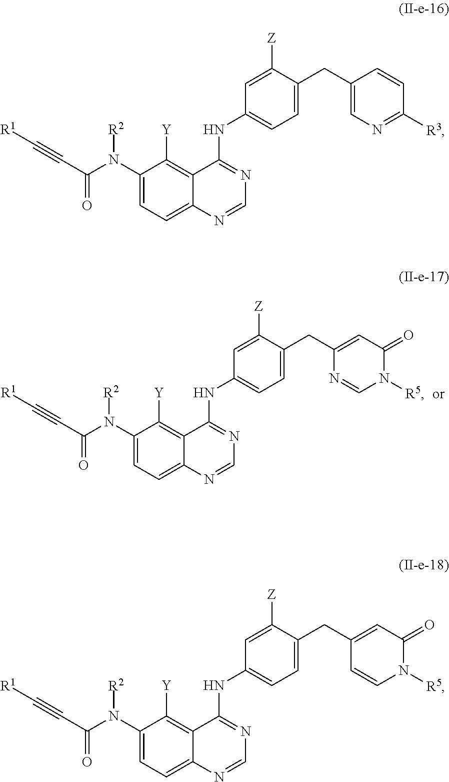

wherein R¹, R², R³, R⁵, Y, and Z are as defined for formula (II). In some embodiments, the compound is a compound of formula (II-e-1). In some embodiments, the compound is a compound of formula (II-e-2). In some embodiments, the compound is a compound of formula (II-e-3). In some embodiments, the compound is a compound of formula (II-e-4). In some embodiments, the compound is a compound of formula (II-e-5). In some embodiments, the compound is a compound of formula (II-e-6). In some embodiments, the compound is a compound of formula (II-e-7). In some embodiments, the compound is a compound of formula (II-e-8). In some embodiments, the compound is a compound of formula (II-e-9). In some embodiments, the compound is a compound of formula (II-e-10). In some embodiments, the compound is a compound of formula (II-e-11). In some embodiments, the compound is a compound of formula (II-e-12). In some embodiments, the compound is a compound of formula (II-e-13). In some embodiments, the compound is a compound of formula (II-e-14). In some embodiments, the compound is a compound of formula (II-e-15). In some embodiments, the compound is a compound of formula (II-e-16). In some embodiments, the compound is a compound of formula (II-e-17). In some embodiments, the compound is a compound of formula (II-e-18). In some embodiments, R¹ is C₁-C₂ alkyl optionally substituted by —OH or —N(C₁-C₃ alkyl)(C₁-C₃ alkyl). In some embodiments, R¹ is unsubstituted C₁-C₂ alkyl. In some embodiments, R¹ is —CH₃. In some embodiments, R¹ is C₁-C₂ alkyl substituted by —N(C₁-C₂ alkyl)(C₁-C₂ alkyl). In some embodiments, R¹ is —CH₂N(CH₃)₂. In some embodiments, R¹ is C₁-C₂ alkyl substituted by —OH. In some embodiments, R¹ is —CH₂CH₂OH. In some embodiments, R² is H, —CH₃, or —CH₂CH₃. In some embodiments, R² is H. In some embodiments, R² is —CH₃. In some embodiments, Y is H, F, or —OCH₃. In some embodiments, Y is H. In some embodiments of the compound of formula (II-e-15) or (II-e-16), R³ is —CH₃, —CH₂CH₃, —CHF₂, or —CD₃. In some embodiments of the compound of formula (II-e-17) or (II-e-18), R⁵ is —CH₃, —CH₂CH₃, —CHF₂, or —CD₃.

In some embodiments, the compound of formula (II-c) is a compound of formula (II-f-1), (II-f-2), (II-f-3), (II-f-4), (II-f-5), (II-f-6), (II-f-7), (II-f-8), (II-f-9), (II-f-10), (II-f-11), (II-f-12), (II-f-13), (II-f-14), (II-f-15), (II-f-16), (II-f-17), or (II-f-18):

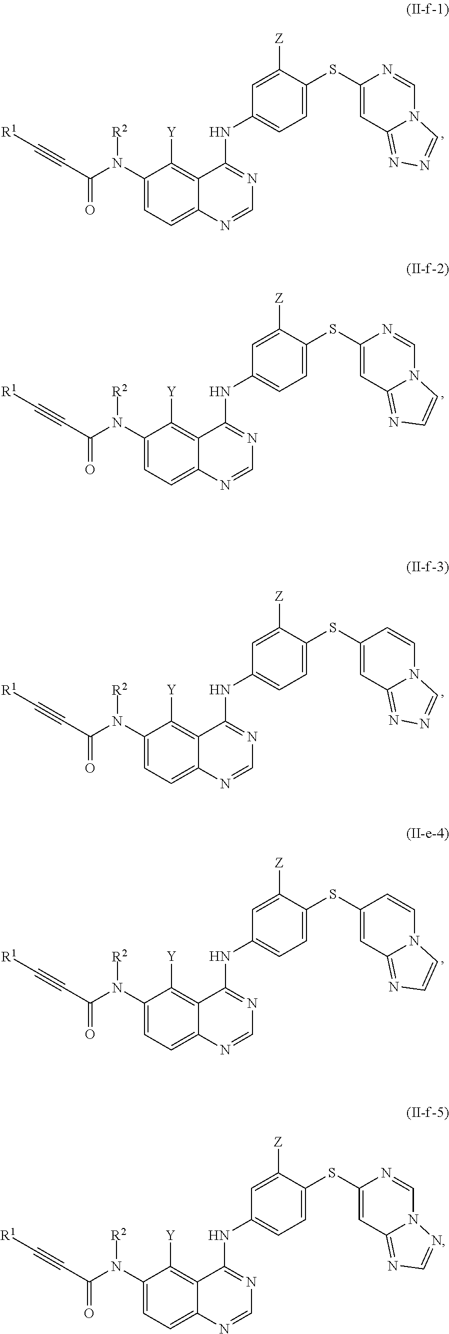

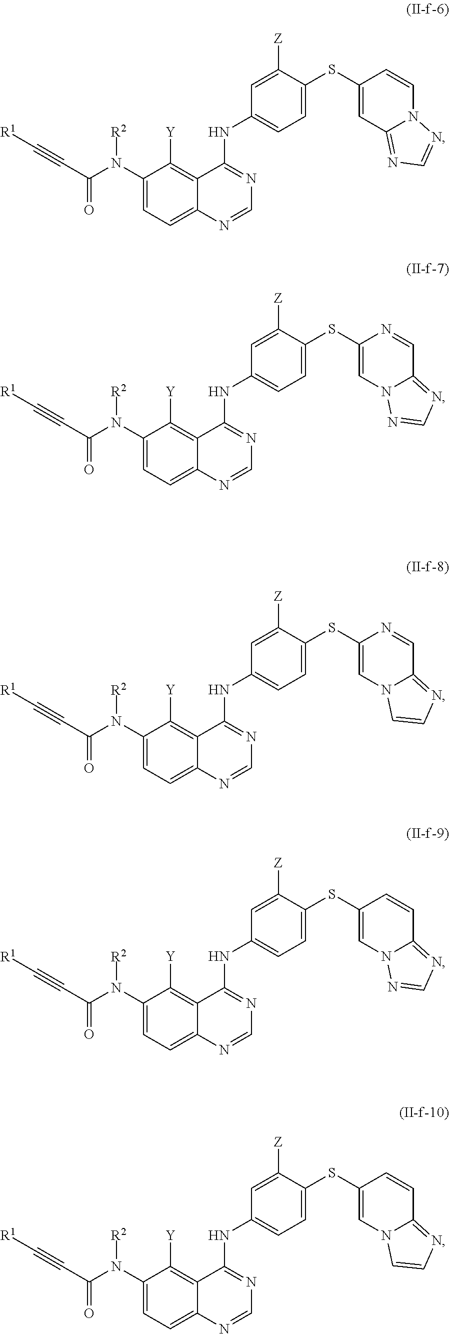

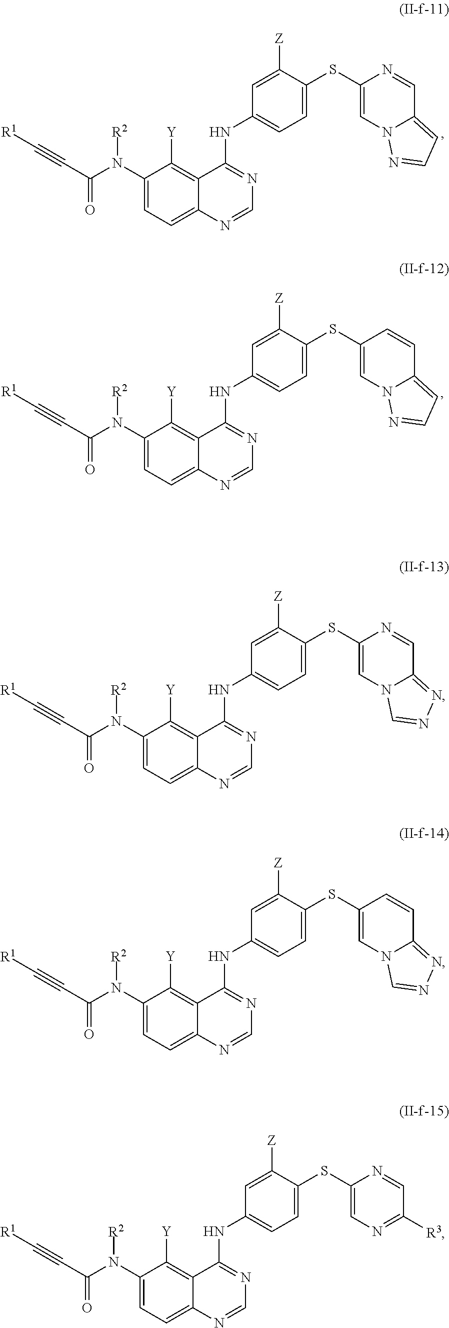

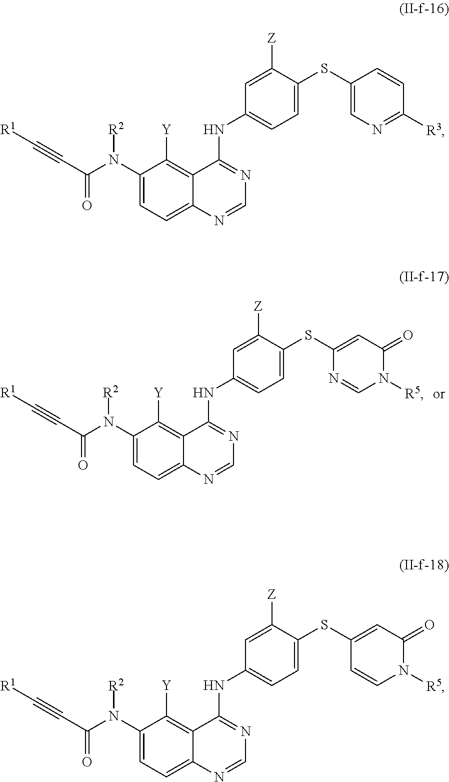

wherein R¹, R², R³, R⁵, Y, and Z are as defined for formula (II). In some embodiments, the compound is a compound of formula (II-f-1). In some embodiments, the compound is a compound of formula (II-f-2). In some embodiments, the compound is a compound of formula (II-f-3). In some embodiments, the compound is a compound of formula (II-f-4). In some embodiments, the compound is a compound of formula (II-f-5). In some embodiments, the compound is a compound of formula (II-f-6). In some embodiments, the compound is a compound of formula (II-f-7). In some embodiments, the compound is a compound of formula (II-f-8). In some embodiments, the compound is a compound of formula (II-f-9). In some embodiments, the compound is a compound of formula (II-f-10). In some embodiments, the compound is a compound of formula (II-f-11). In some embodiments, the compound is a compound of formula (II-f-12). In some embodiments, the compound is a compound of formula (II-f-13). In some embodiments, the compound is a compound of formula (II-f-14). In some embodiments, the compound is a compound of formula (II-f-15). In some embodiments, the compound is a compound of formula (II-f-16). In some embodiments, the compound is a compound of formula (II-f-17). In some embodiments, the compound is a compound of formula (II-f-18). In some embodiments, R¹ is C₁-C₂ alkyl optionally substituted by —OH or —N(C₁-C₃ alkyl)(C₁-C₃ alkyl). In some embodiments, R¹ is unsubstituted C₁-C₂ alkyl. In some embodiments, R¹ is —CH₃. In some embodiments, R¹ is C₁-C₂ alkyl substituted by —N(C₁-C₂ alkyl)(C₁-C₂ alkyl). In some embodiments, R¹ is —CH₂N(CH₃)₂. In some embodiments, R¹ is C₁-C₂ alkyl substituted by —OH. In some embodiments, R¹ is —CH₂CH₂OH. In some embodiments, R² is H, —CH₃, or —CH₂CH₃. In some embodiments, R² is H. In some embodiments, R² is —CH₃. In some embodiments, Y is H, F, or —OCH₃. In some embodiments, Y is H. In some embodiments of the compound of formula (II-f-15) or (II-f-16), R³ is —CH₃, —CH₂CH₃, —CHF₂, or —CD₃. In some embodiments of the compound of formula (II-f-17) or (II-f-18), R⁵ is —CH₃, —CH₂CH₃, —CHF₂, or —CD₃.

In some embodiments, the compound of formula (II-a) is a compound of formula (II-g-1), (II-g-2), (II-g-3), (II-g-4), (II-g-5), or (II-g-6):

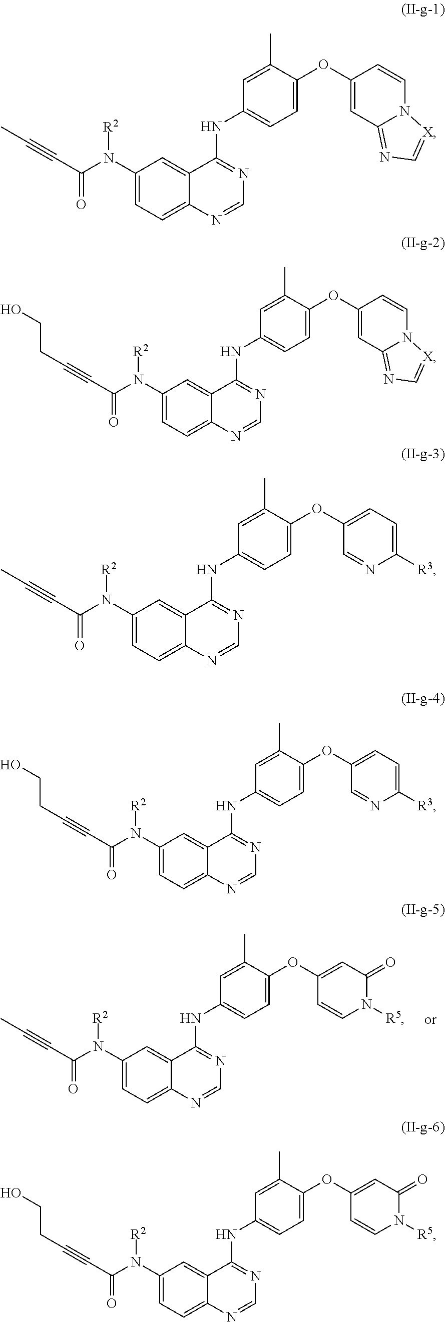

wherein R¹, R², R³, R⁵, and X are as defined for formula (II). In some embodiments, the compound is a compound of formula (II-g-1). In some embodiments, the compound is a compound of formula (II-g-2). In some embodiments, the compound is a compound of formula (II-g-3). In some embodiments, the compound is a compound of formula (II-g-4). In some embodiments, the compound is a compound of formula (II-g-5). In some embodiments, the compound is a compound of formula (II-g-6). In some embodiments, R² is H, —CH₃, or —CH₂CH₃. In some embodiments, R² is H. In some embodiments, R² is —CH₃. In some embodiments of the compound of formula (II-g-1) or (II-g-2), X is CH. In some embodiments of the compound of formula (II-g-1) or (II-g-2), X is N. In some embodiments of the compound of formula (II-g-3) or (II-g-4), R³ is —CH₃, —CH₂CH₃, —CHF₂, or —CD₃. In some embodiments of the compound of formula (II-g-5) or (II-g-6), R⁵ is —CH₃, —CH₂CH₃, —CHF₂, or —CD₃.

In some embodiments, provided is a compound of formula (II) selected from the compounds in Table 1, or pharmaceutically acceptable salt thereof. Although certain compounds described in Table 1 may be presented as specific stereoisomers and/or in a non-stereochemical form, it is understood that any or all stereochemical forms, including any enantiomeric or diastereomeric forms, and any tautomers or other forms of any of the compounds of Table 1 are herein described.

TABLE 1

| Compound | |
|---|---|
| No. | Structure |
| 1 | 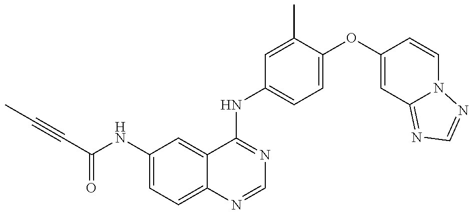 [SMILES: CC#CC(=O)Nc1ccc2ncnc(Nc3ccc(Oc4ccn5ncnc5c4)c(C)c3)c2c1] |
| 2 | 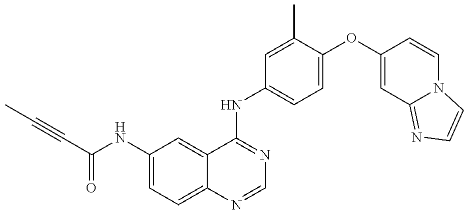 [SMILES: CC#CC(=O)Nc1ccc2ncnc(Nc3ccc(Oc4ccn5ccnc5c4)c(C)c3)c2c1] |
| 3 | 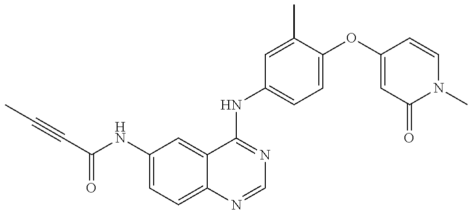 [SMILES: CC#CC(=O)Nc1ccc2ncnc(Nc3ccc(Oc4ccn(C)c(=O)c4)c(C)c3)c2c1] |
| 4 | 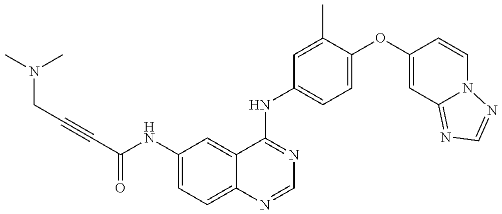 [SMILES: Cc1cc(Nc2ncnc3ccc(NC(=O)C#CCN(C)C)cc23)ccc1Oc1ccn2ncnc2c1] |
| 5 | 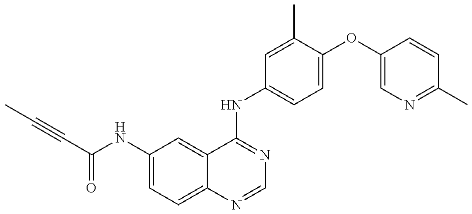 [SMILES: CC#CC(=O)Nc1ccc2ncnc(Nc3ccc(Oc4ccc(C)nc4)c(C)c3)c2c1] |
| 6 | 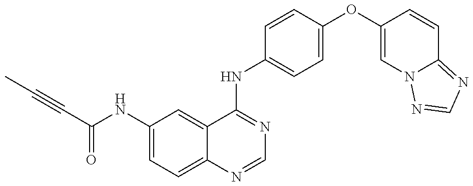 [SMILES: CC#CC(=O)Nc1ccc2ncnc(Nc3ccc(Oc4ccc5ncnn5c4)cc3)c2c1] |
| 7 | 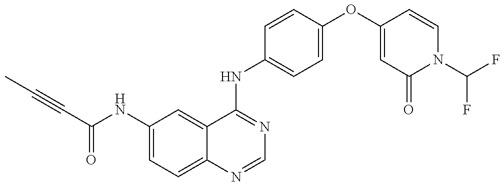 [SMILES: CC#CC(=O)Nc1ccc2ncnc(Nc3ccc(Oc4ccn(C(F)F)c(=O)c4)cc3)c2c1] |
| 8 | 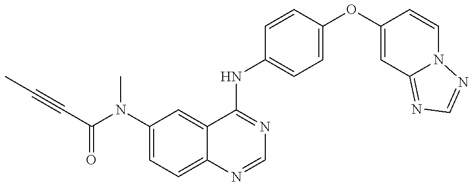 [SMILES: CC#CC(=O)N(C)c1ccc2ncnc(Nc3ccc(Oc4ccn5ncnc5c4)cc3)c2c1] |
| 9 | 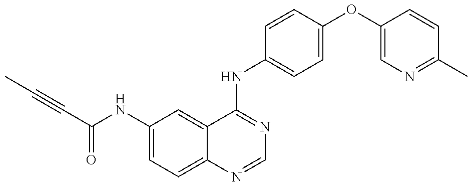 [SMILES: CC#CC(=O)Nc1ccc2ncnc(Nc3ccc(Oc4ccc(C)nc4)cc3)c2c1] |
| 10 | 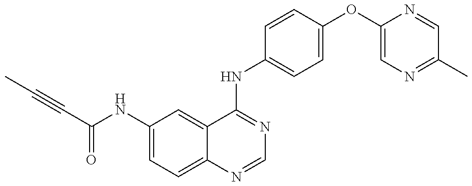 [SMILES: CC#CC(=O)Nc1ccc2ncnc(Nc3ccc(Oc4cnc(C)cn4)cc3)c2c1] |
| 11 | 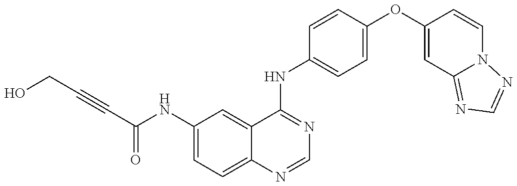 [SMILES: O=C(C#CCO)Nc1ccc2ncnc(Nc3ccc(Oc4ccn5ncnc5c4)cc3)c2c1] |
| 12 | 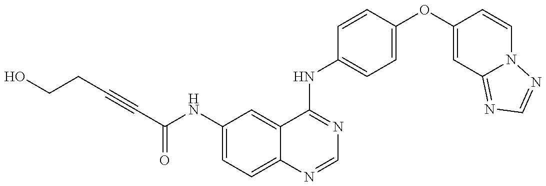 [SMILES: O=C(C#CCCO)Nc1ccc2ncnc(Nc3ccc(Oc4ccn5ncnc5c4)cc3)c2c1] |
| 13 | 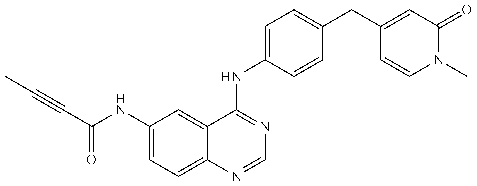 [SMILES: CC#CC(=O)Nc1ccc2ncnc(Nc3ccc(Cc4ccn(C)c(=O)c4)cc3)c2c1] |
| 14 | 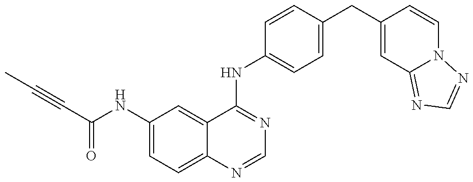 [SMILES: CC#CC(=O)Nc1ccc2ncnc(Nc3ccc(Cc4ccn5ncnc5c4)cc3)c2c1] |
| 15 | 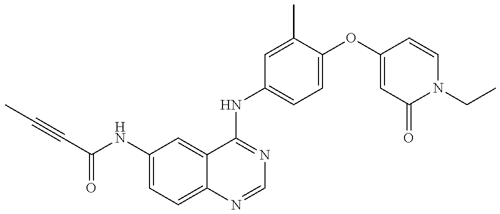 [SMILES: CC#CC(=O)Nc1ccc2ncnc(Nc3ccc(Oc4ccn(CC)c(=O)c4)c(C)c3)c2c1] |
| 16 | 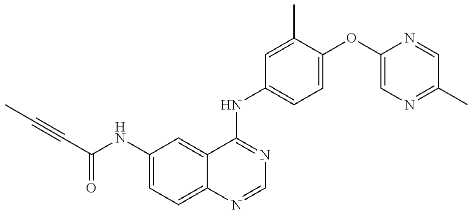 [SMILES: CC#CC(=O)Nc1ccc2ncnc(Nc3ccc(Oc4cnc(C)cn4)c(C)c3)c2c1] |
| 17 | 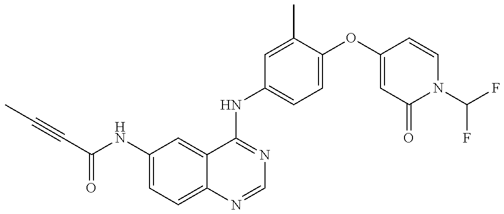 [SMILES: CC#CC(=O)Nc1ccc2ncnc(Nc3ccc(Oc4ccn(C(F)F)c(=O)c4)c(C)c3)c2c1] |
| 18 | 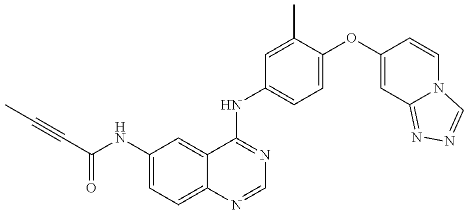 [SMILES: CC#CC(=O)Nc1ccc2ncnc(Nc3ccc(Oc4ccn5cnnc5c4)c(C)c3)c2c1] |
| 19 | 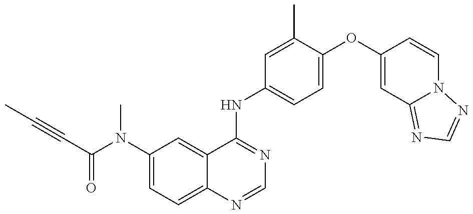 [SMILES: CC#CC(=O)N(C)c1ccc2ncnc(Nc3ccc(Oc4ccn5ncnc5c4)c(C)c3)c2c1] |
| 20 | 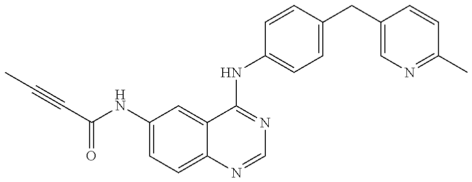 [SMILES: CC#CC(=O)Nc1ccc2ncnc(Nc3ccc(Cc4ccc(C)nc4)cc3)c2c1] |
| 21 | 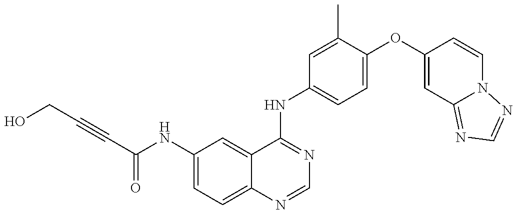 [SMILES: Cc1cc(Nc2ncnc3ccc(NC(=O)C#CCO)cc23)ccc1Oc1ccn2ncnc2c1] |
| 22 | 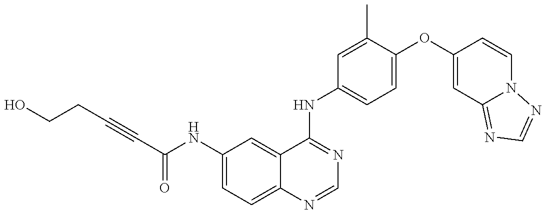 [SMILES: Cc1cc(Nc2ncnc3ccc(NC(=O)C#CCCO)cc23)ccc1Oc1ccn2ncnc2c1] |
| 23 | 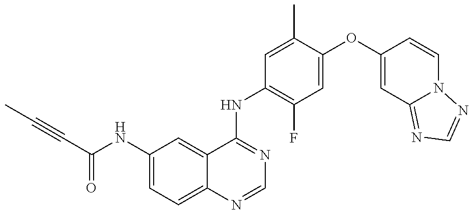 [SMILES: CC#CC(=O)Nc1ccc2ncnc(Nc3cc(C)c(Oc4ccn5ncnc5c4)cc3F)c2c1] |
| 24 | 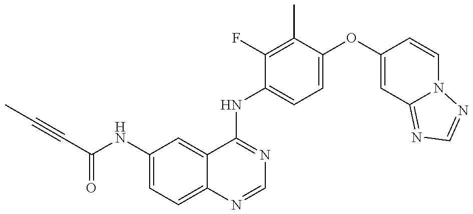 [SMILES: CC#CC(=O)Nc1ccc2ncnc(Nc3ccc(Oc4ccn5ncnc5c4)c(C)c3F)c2c1] |
| 25 | 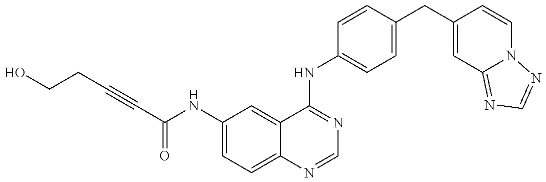 [SMILES: O=C(C#CCCO)Nc1ccc2ncnc(Nc3ccc(Cc4ccn5ncnc5c4)cc3)c2c1] |
| 26 | 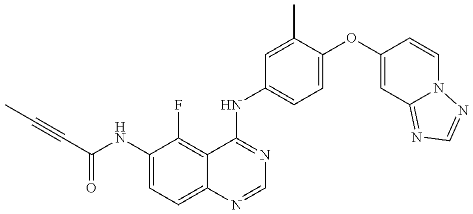 [SMILES: CC#CC(=O)Nc1ccc2ncnc(Nc3ccc(Oc4ccn5ncnc5c4)c(C)c3)c2c1F] |
| 27 | 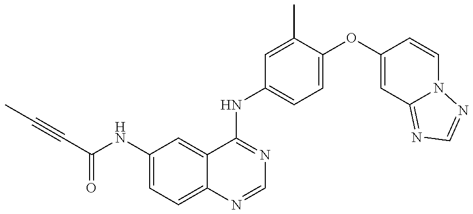 [SMILES: CC#CC(=O)Nc1ccc2ncnc(Nc3ccc(Oc4ccn5ncnc5c4)c(C)c3)c2c1] |
| 28 | 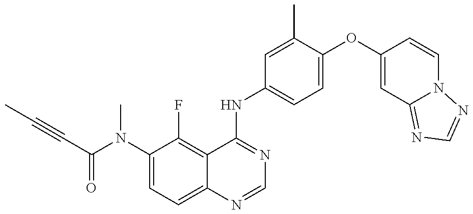 [SMILES: CC#CC(=O)N(C)c1ccc2ncnc(Nc3ccc(Oc4ccn5ncnc5c4)c(C)c3)c2c1F] |
| 29 | 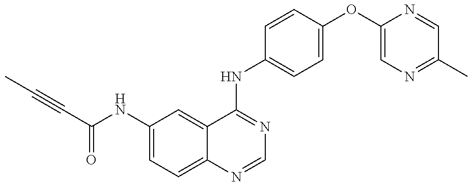 [SMILES: CC#CC(=O)Nc1ccc2ncnc(Nc3ccc(Oc4cnc(C)cn4)cc3)c2c1] |
| 30 | 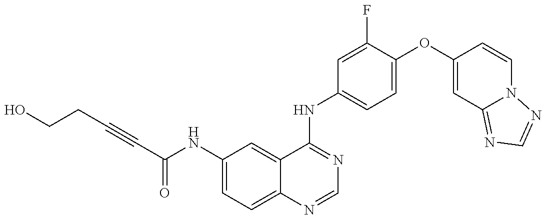 [SMILES: O=C(C#CCCO)Nc1ccc2ncnc(Nc3ccc(Oc4ccn5ncnc5c4)c(F)c3)c2c1] |
| 31 | 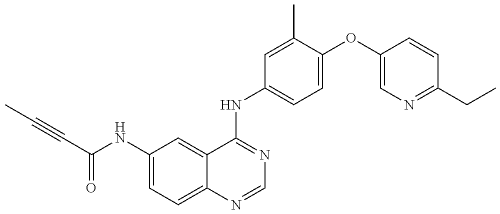 [SMILES: CC#CC(=O)Nc1ccc2ncnc(Nc3ccc(Oc4ccc(CC)nc4)c(C)c3)c2c1] |
| 32 | 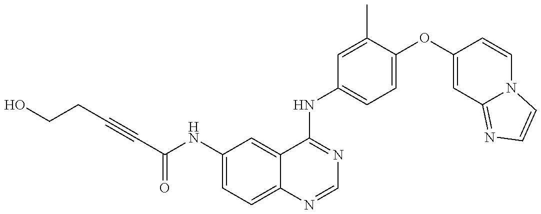 [SMILES: Cc1cc(Nc2ncnc3ccc(NC(=O)C#CCCO)cc23)ccc1Oc1ccn2ccnc2c1] |
| 33 | 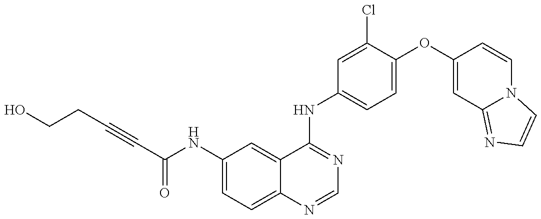 [SMILES: O=C(C#CCCO)Nc1ccc2ncnc(Nc3ccc(Oc4ccn5ccnc5c4)c(Cl)c3)c2c1] |
| 34 | 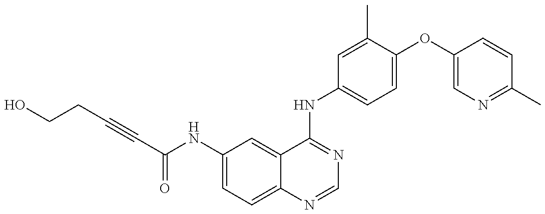 [SMILES: Cc1ccc(Oc2ccc(Nc3ncnc4ccc(NC(=O)C#CCCO)cc34)cc2C)cn1] |
| 35 | 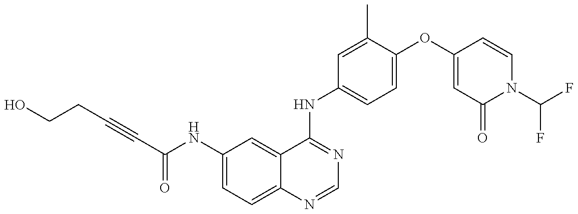 [SMILES: Cc1cc(Nc2ncnc3ccc(NC(=O)C#CCCO)cc23)ccc1Oc1ccn(C(F)F)c(=O)c1] |
| 36 | 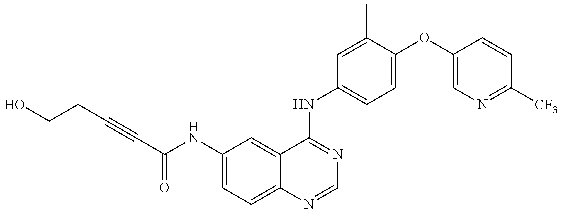 [SMILES: Cc1cc(Nc2ncnc3ccc(NC(=O)C#CCCO)cc23)ccc1Oc1ccc(C(F)(F)F)nc1] |
| 37 | 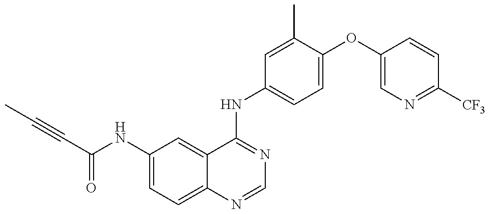 [SMILES: CC#CC(=O)Nc1ccc2ncnc(Nc3ccc(Oc4ccc(C(F)(F)F)nc4)c(C)c3)c2c1] |
| 38 | 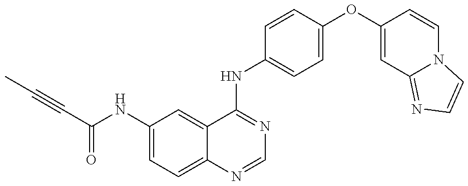 [SMILES: CC#CC(=O)Nc1ccc2ncnc(Nc3ccc(Oc4ccn5ccnc5c4)cc3)c2c1] |
| 39 | 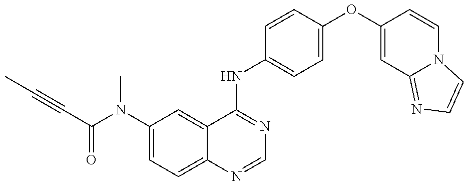 [SMILES: CC#CC(=O)N(C)c1ccc2ncnc(Nc3ccc(Oc4ccn5ccnc5c4)cc3)c2c1] |
| 40 | 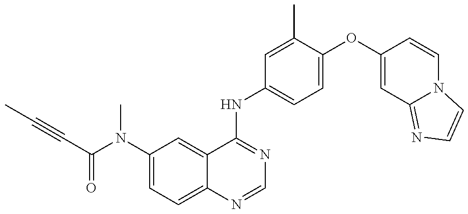 [SMILES: CC#CC(=O)N(C)c1ccc2ncnc(Nc3ccc(Oc4ccn5ccnc5c4)c(C)c3)c2c1] |
| 41 | 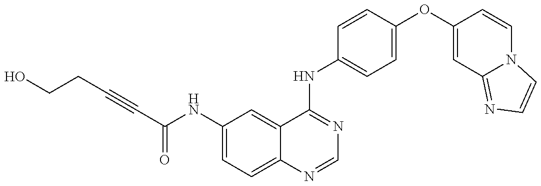 [SMILES: O=C(C#CCCO)Nc1ccc2ncnc(Nc3ccc(Oc4ccn5ccnc5c4)cc3)c2c1] |
| 42 | 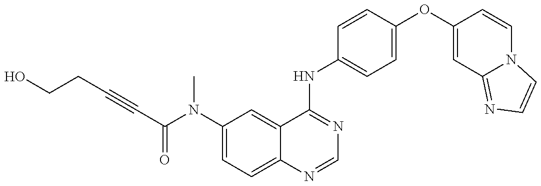 [SMILES: CN(C(=O)C#CCCO)c1ccc2ncnc(Nc3ccc(Oc4ccn5ccnc5c4)cc3)c2c1] |
| 43 | 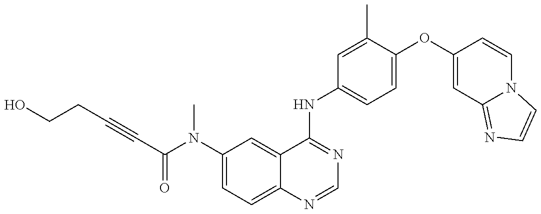 [SMILES: Cc1cc(Nc2ncnc3ccc(N(C)C(=O)C#CCCO)cc23)ccc1Oc1ccn2ccnc2c1] |
| 44 | 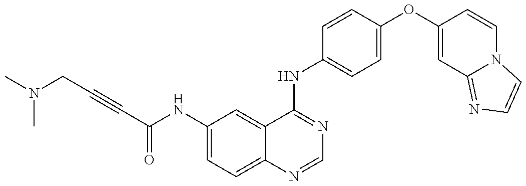 [SMILES: CN(C)CC#CC(=O)Nc1ccc2ncnc(Nc3ccc(Oc4ccn5ccnc5c4)cc3)c2c1] |
| 45 | 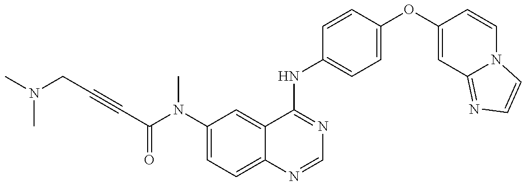 [SMILES: CN(C)CC#CC(=O)N(C)c1ccc2ncnc(Nc3ccc(Oc4ccn5ccnc5c4)cc3)c2c1] |
| 46 | 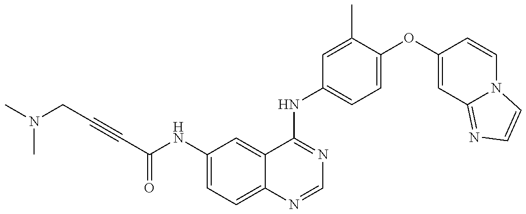 [SMILES: Cc1cc(Nc2ncnc3ccc(NC(=O)C#CCN(C)C)cc23)ccc1Oc1ccn2ccnc2c1] |
| 47 | 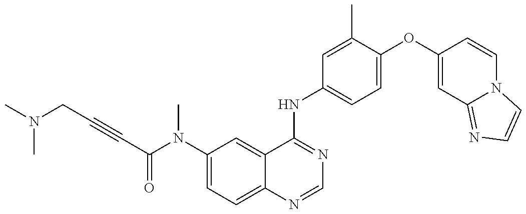 [SMILES: Cc1cc(Nc2ncnc3ccc(N(C)C(=O)C#CCN(C)C)cc23)ccc1Oc1ccn2ccnc2c1] |
| 48 | 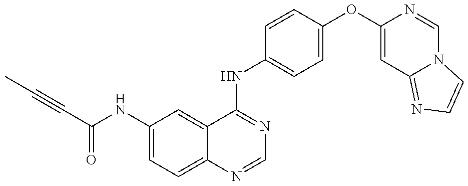 [SMILES: CC#CC(=O)Nc1ccc2ncnc(Nc3ccc(Oc4cc5nccn5cn4)cc3)c2c1] |
| 49 | 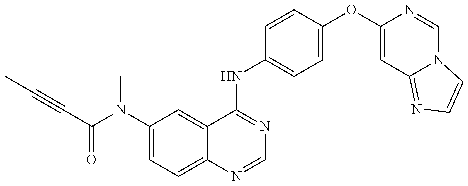 [SMILES: CC#CC(=O)N(C)c1ccc2ncnc(Nc3ccc(Oc4cc5nccn5cn4)cc3)c2c1] |
| 50 | 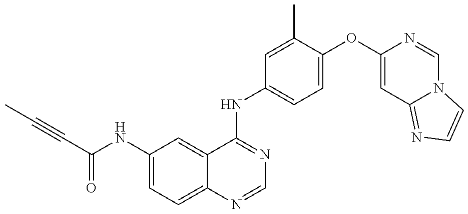 [SMILES: CC#CC(=O)Nc1ccc2ncnc(Nc3ccc(Oc4cc5nccn5cn4)c(C)c3)c2c1] |
| 51 | 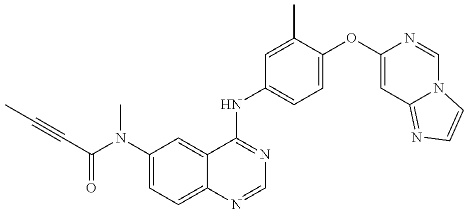 [SMILES: CC#CC(=O)N(C)c1ccc2ncnc(Nc3ccc(Oc4cc5nccn5cn4)c(C)c3)c2c1] |
| 52 | 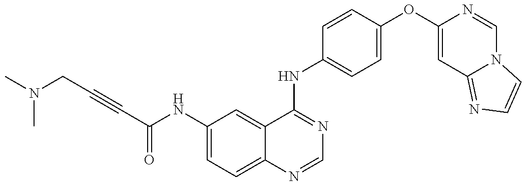 [SMILES: CN(C)CC#CC(=O)Nc1ccc2ncnc(Nc3ccc(Oc4cc5nccn5cn4)cc3)c2c1] |
| 53 | 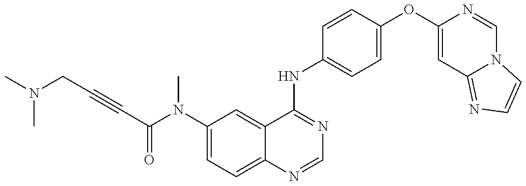 [SMILES: CN(C)CC#CC(=O)N(C)c1ccc2ncnc(Nc3ccc(Oc4cc5nccn5cn4)cc3)c2c1] |
| 54 | 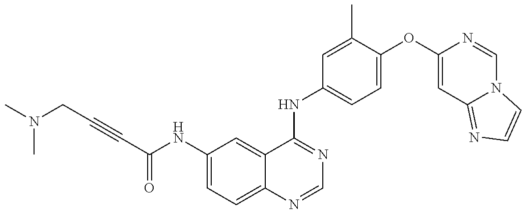 [SMILES: Cc1cc(Nc2ncnc3ccc(NC(=O)C#CCN(C)C)cc23)ccc1Oc1cc2nccn2cn1] |
| 55 | 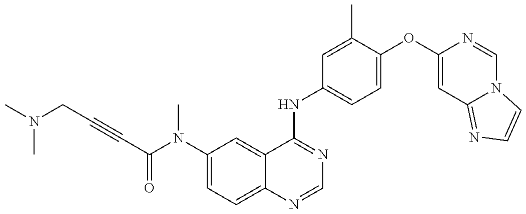 [SMILES: Cc1cc(Nc2ncnc3ccc(N(C)C(=O)C#CCN(C)C)cc23)ccc1Oc1cc2nccn2cn1] |
| 56 | 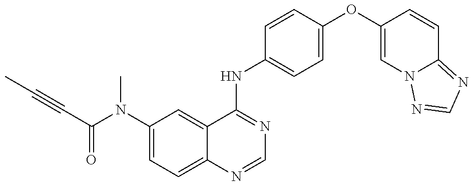 [SMILES: CC#CC(=O)N(C)c1ccc2ncnc(Nc3ccc(Oc4ccc5ncnn5c4)cc3)c2c1] |
| 57 | 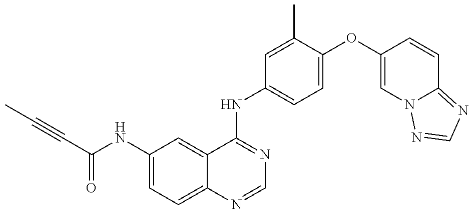 [SMILES: CC#CC(=O)Nc1ccc2ncnc(Nc3ccc(Oc4ccc5ncnn5c4)c(C)c3)c2c1] |
| 58 | 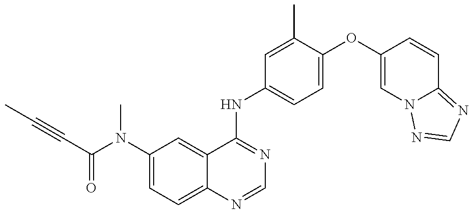 [SMILES: CC#CC(=O)N(C)c1ccc2ncnc(Nc3ccc(Oc4ccc5ncnn5c4)c(C)c3)c2c1] |
| 59 | 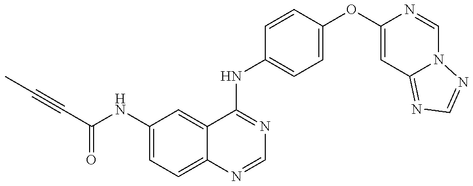 [SMILES: CC#CC(=O)Nc1ccc2ncnc(Nc3ccc(Oc4cc5ncnn5cn4)cc3)c2c1] |
| 60 | 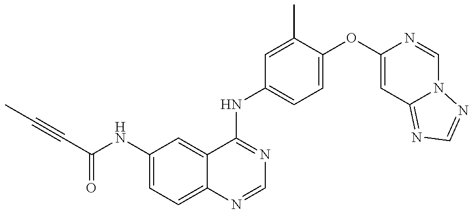 [SMILES: CC#CC(=O)Nc1ccc2ncnc(Nc3ccc(Oc4cc5ncnn5cn4)c(C)c3)c2c1] |
| 61 | 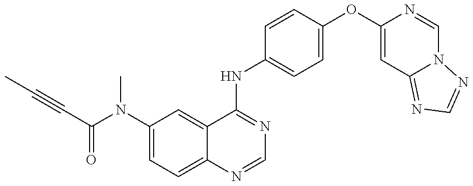 [SMILES: CC#CC(=O)N(C)c1ccc2ncnc(Nc3ccc(Oc4cc5ncnn5cn4)cc3)c2c1] |
| 62 | 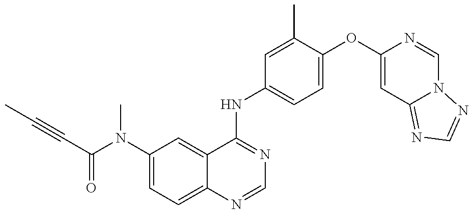 [SMILES: CC#CC(=O)N(C)c1ccc2ncnc(Nc3ccc(Oc4cc5ncnn5cn4)c(C)c3)c2c1] |
| 63 | 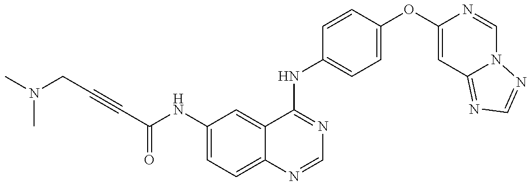 [SMILES: CN(C)CC#CC(=O)Nc1ccc2ncnc(Nc3ccc(Oc4cc5ncnn5cn4)cc3)c2c1] |
| 64 | 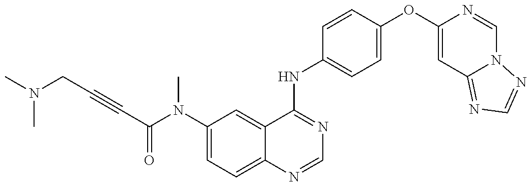 [SMILES: CN(C)CC#CC(=O)N(C)c1ccc2ncnc(Nc3ccc(Oc4cc5ncnn5cn4)cc3)c2c1] |
| 65 | 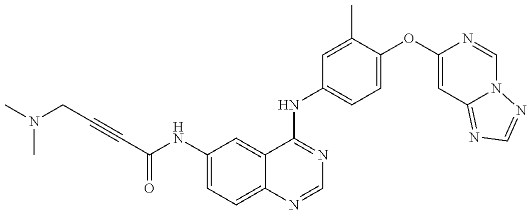 [SMILES: Cc1cc(Nc2ncnc3ccc(NC(=O)C#CCN(C)C)cc23)ccc1Oc1cc2ncnn2cn1] |
| 66 | 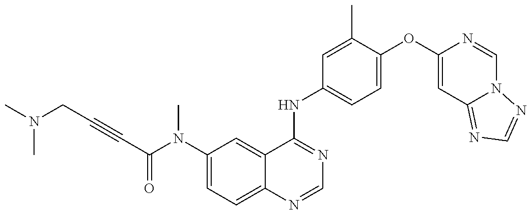 [SMILES: Cc1cc(Nc2ncnc3ccc(N(C)C(=O)C#CCN(C)C)cc23)ccc1Oc1cc2ncnn2cn1] |
| 67 | 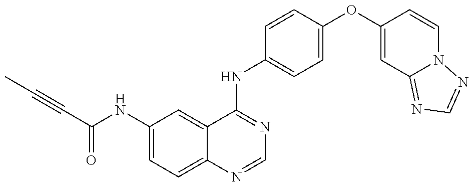 [SMILES: CC#CC(=O)Nc1ccc2ncnc(Nc3ccc(Oc4ccn5ncnc5c4)cc3)c2c1] |
| 68 | 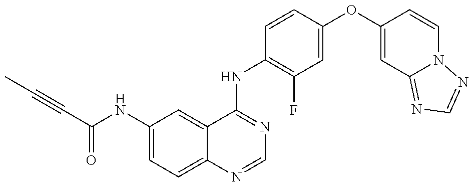 [SMILES: CC#CC(=O)Nc1ccc2ncnc(Nc3ccc(Oc4ccn5ncnc5c4)cc3F)c2c1] |
| 69 | 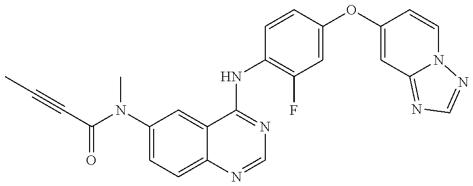 [SMILES: CC#CC(=O)N(C)c1ccc2ncnc(Nc3ccc(Oc4ccn5ncnc5c4)cc3F)c2c1] |
| 70 | 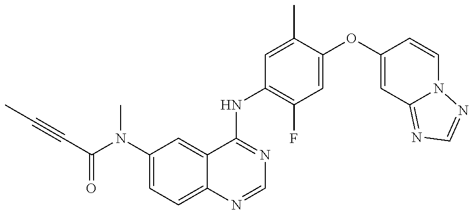 [SMILES: CC#CC(=O)N(C)c1ccc2ncnc(Nc3cc(C)c(Oc4ccn5ncnc5c4)cc3F)c2c1] |
| 71 | 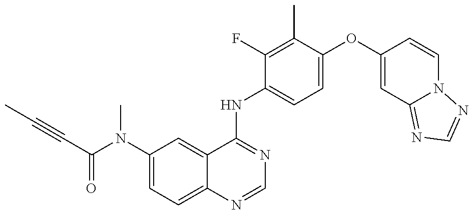 [SMILES: CC#CC(=O)N(C)c1ccc2ncnc(Nc3ccc(Oc4ccn5ncnc5c4)c(C)c3F)c2c1] |
| 72 | 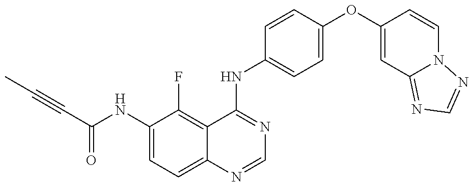 [SMILES: CC#CC(=O)Nc1ccc2ncnc(Nc3ccc(Oc4ccn5ncnc5c4)cc3)c2c1F] |
| 73 | 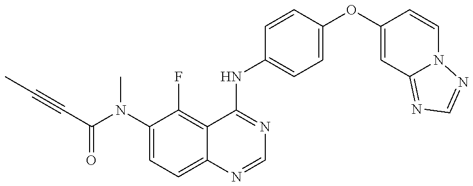 [SMILES: CC#CC(=O)N(C)c1ccc2ncnc(Nc3ccc(Oc4ccn5ncnc5c4)cc3)c2c1F] |
| 74 | 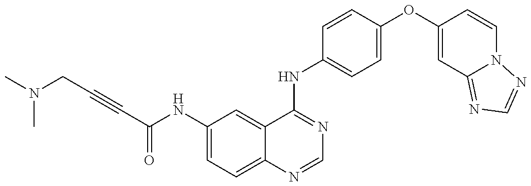 [SMILES: CN(C)CC#CC(=O)Nc1ccc2ncnc(Nc3ccc(Oc4ccn5ncnc5c4)cc3)c2c1] |
| 75 | 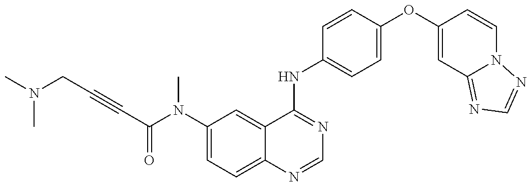 [SMILES: CN(C)CC#CC(=O)N(C)c1ccc2ncnc(Nc3ccc(Oc4ccn5ncnc5c4)cc3)c2c1] |
| 76 | 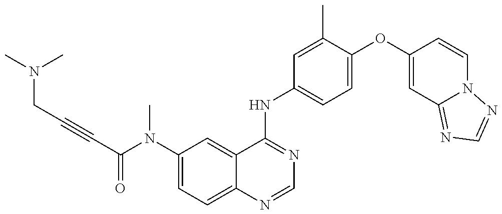 [SMILES: Cc1cc(Nc2ncnc3ccc(N(C)C(=O)C#CCN(C)C)cc23)ccc1Oc1ccn2ncnc2c1] |
| 77 | 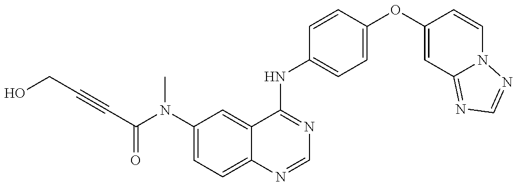 [SMILES: CN(C(=O)C#CCO)c1ccc2ncnc(Nc3ccc(Oc4ccn5ncnc5c4)cc3)c2c1] |
| 78 | 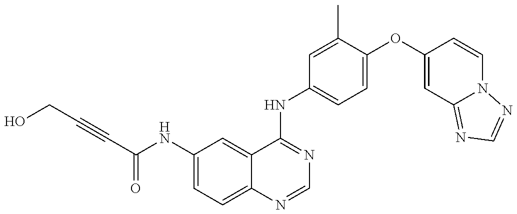 [SMILES: Cc1cc(Nc2ncnc3ccc(NC(=O)C#CCO)cc23)ccc1Oc1ccn2ncnc2c1] |
| 79 | 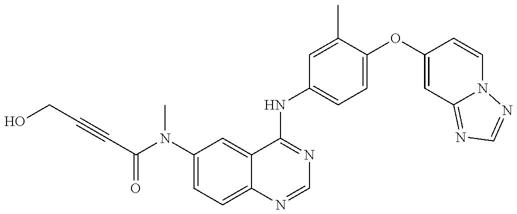 [SMILES: Cc1cc(Nc2ncnc3ccc(N(C)C(=O)C#CCO)cc23)ccc1Oc1ccn2ncnc2c1] |
| 80 | 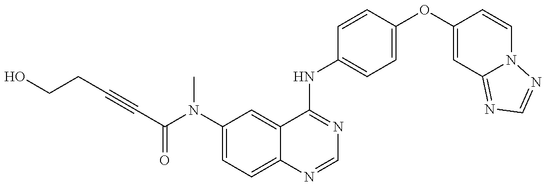 [SMILES: CN(C(=O)C#CCCO)c1ccc2ncnc(Nc3ccc(Oc4ccn5ncnc5c4)cc3)c2c1] |
| 81 | 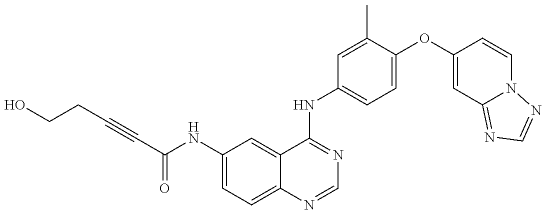 [SMILES: Cc1cc(Nc2ncnc3ccc(NC(=O)C#CCCO)cc23)ccc1Oc1ccn2ncnc2c1] |
| 82 | 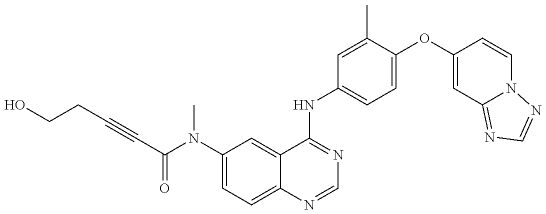 [SMILES: Cc1cc(Nc2ncnc3ccc(N(C)C(=O)C#CCCO)cc23)ccc1Oc1ccn2ncnc2c1] |
| 83 | 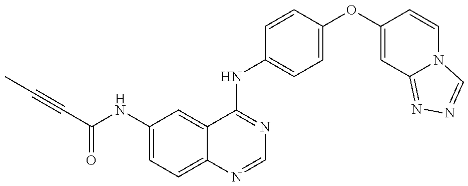 [SMILES: CC#CC(=O)Nc1ccc2ncnc(Nc3ccc(Oc4ccn5cnnc5c4)cc3)c2c1] |
| 84 | 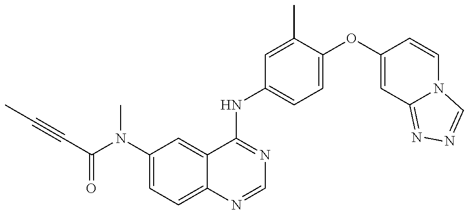 [SMILES: CC#CC(=O)N(C)c1ccc2ncnc(Nc3ccc(Oc4ccn5cnnc5c4)c(C)c3)c2c1] |
| 85 | 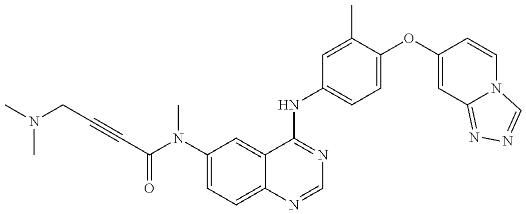 [SMILES: Cc1cc(Nc2ncnc3ccc(N(C)C(=O)C#CCN(C)C)cc23)ccc1Oc1ccn2cnnc2c1] |
| 86 | 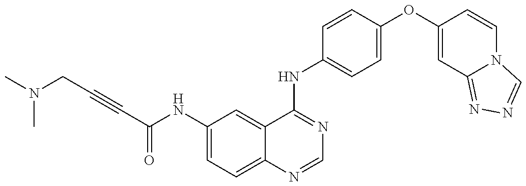 [SMILES: CN(C)CC#CC(=O)Nc1ccc2ncnc(Nc3ccc(Oc4ccn5cnnc5c4)cc3)c2c1] |
| 87 | 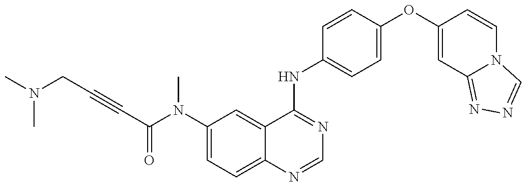 [SMILES: CN(C)CC#CC(=O)N(C)c1ccc2ncnc(Nc3ccc(Oc4ccn5cnnc5c4)cc3)c2c1] |
| 88 | 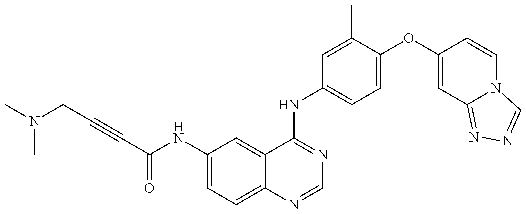 [SMILES: Cc1cc(Nc2ncnc3ccc(NC(=O)C#CCN(C)C)cc23)ccc1Oc1ccn2cnnc2c1] |
| 89 | 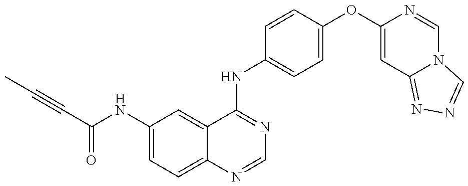 [SMILES: CC#CC(=O)Nc1ccc2ncnc(Nc3ccc(Oc4cc5nncn5cn4)cc3)c2c1] |
| 90 | 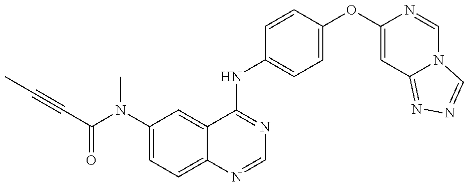 [SMILES: CC#CC(=O)N(C)c1ccc2ncnc(Nc3ccc(Oc4cc5nncn5cn4)cc3)c2c1] |
| 91 | 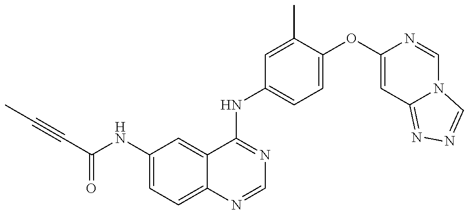 [SMILES: CC#CC(=O)Nc1ccc2ncnc(Nc3ccc(Oc4cc5nncn5cn4)c(C)c3)c2c1] |
| 92 | 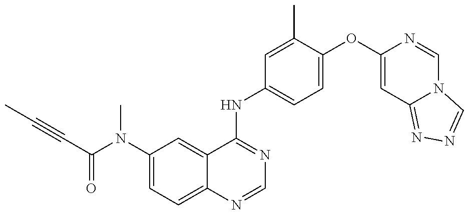 [SMILES: CC#CC(=O)N(C)c1ccc2ncnc(Nc3ccc(Oc4cc5nncn5cn4)c(C)c3)c2c1] |
| 93 | 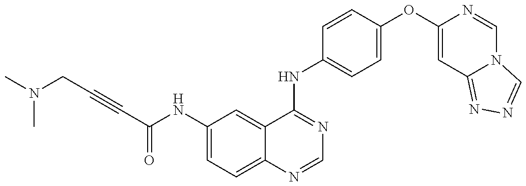 [SMILES: CN(C)CC#CC(=O)Nc1ccc2ncnc(Nc3ccc(Oc4cc5nncn5cn4)cc3)c2c1] |
| 94 | 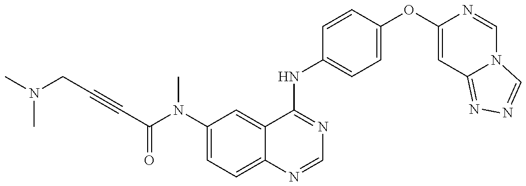 [SMILES: CN(C)CC#CC(=O)N(C)c1ccc2ncnc(Nc3ccc(Oc4cc5nncn5cn4)cc3)c2c1] |
| 95 | 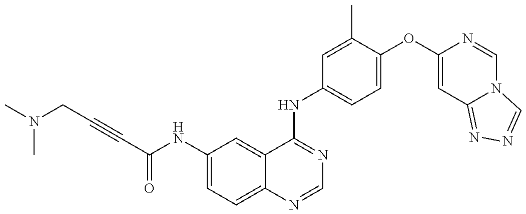 [SMILES: Cc1cc(Nc2ncnc3ccc(NC(=O)C#CCN(C)C)cc23)ccc1Oc1cc2nncn2cn1] |
| 96 | 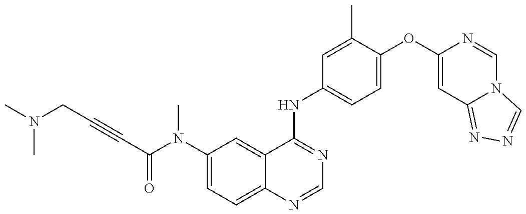 [SMILES: Cc1cc(Nc2ncnc3ccc(N(C)C(=O)C#CCN(C)C)cc23)ccc1Oc1cc2nncn2cn1] |
| 97 | 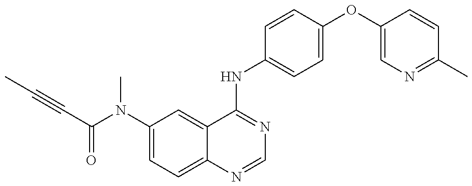 [SMILES: CC#CC(=O)N(C)c1ccc2ncnc(Nc3ccc(Oc4ccc(C)nc4)cc3)c2c1] |
| 98 | 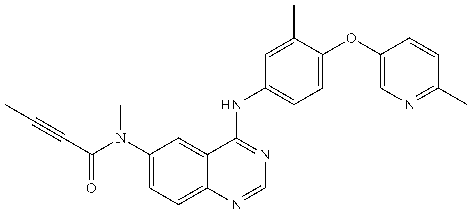 [SMILES: CC#CC(=O)N(C)c1ccc2ncnc(Nc3ccc(Oc4ccc(C)nc4)c(C)c3)c2c1] |
| 99 | 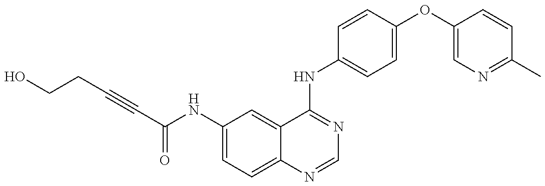 [SMILES: Cc1ccc(Oc2ccc(Nc3ncnc4ccc(NC(=O)C#CCCO)cc34)cc2)cn1] |
| 100 | 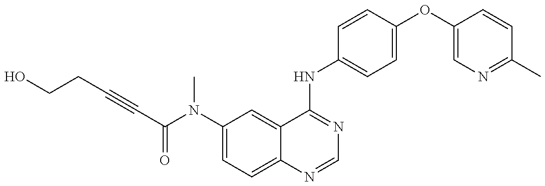 [SMILES: Cc1ccc(Oc2ccc(Nc3ncnc4ccc(N(C)C(=O)C#CCCO)cc34)cc2)cn1] |
| 101 | 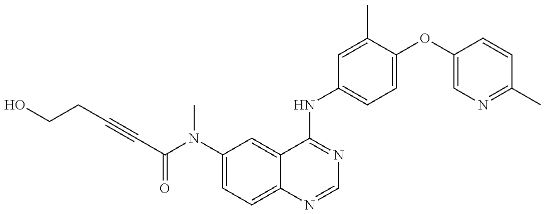 [SMILES: Cc1ccc(Oc2ccc(Nc3ncnc4ccc(N(C)C(=O)C#CCCO)cc34)cc2C)cn1] |
| 102 | 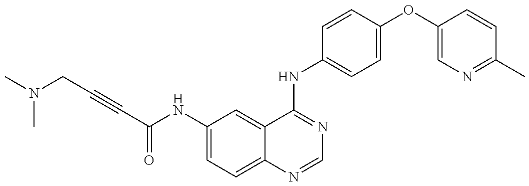 |
| 103 | 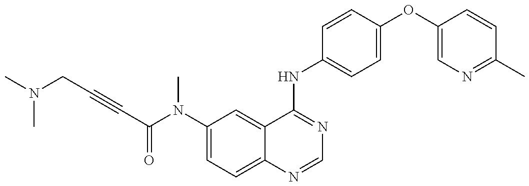 |
| 104 | 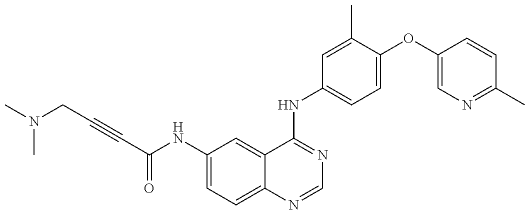 |
| 105 | 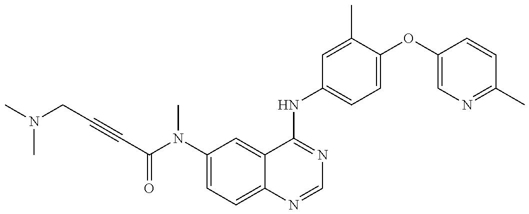 |
| 106 | 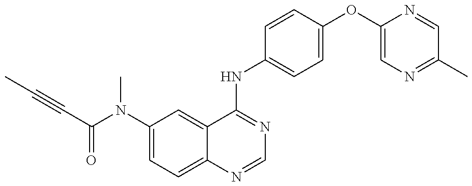 [SMILES: CC#CC(=O)N(C)c1ccc2ncnc(Nc3ccc(Oc4cnc(C)cn4)cc3)c2c1] |
| 107 | 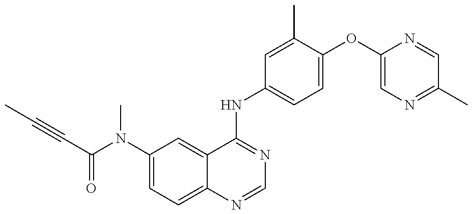 [SMILES: CC#CC(=O)N(C)c1ccc2ncnc(Nc3ccc(Oc4cnc(C)cn4)c(C)c3)c2c1] |
| 108 | 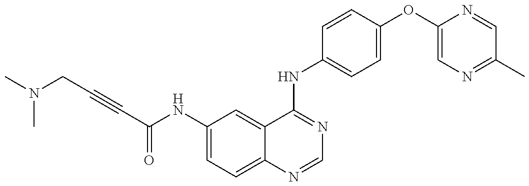 |
| 109 | 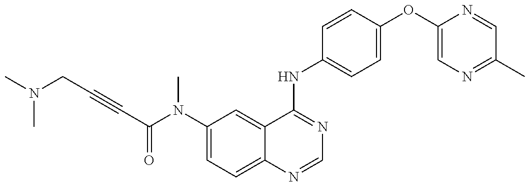 |
| 110 | 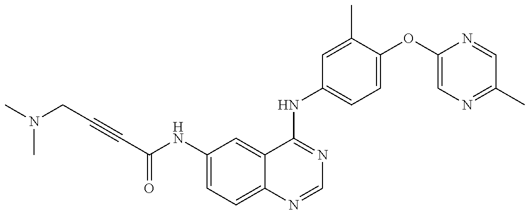 |
| 111 | 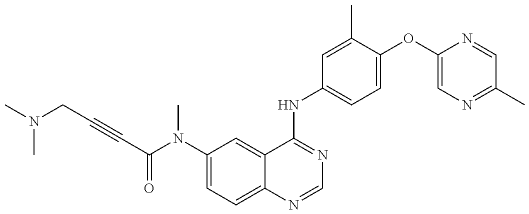 |
| 112 | 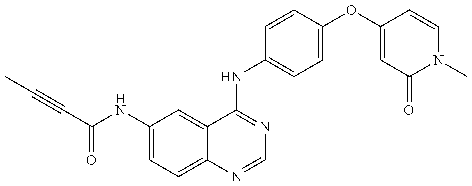 [SMILES: CC#CC(=O)Nc1ccc2ncnc(Nc3ccc(Oc4ccn(C)c(=O)c4)cc3)c2c1] |
| 113 | 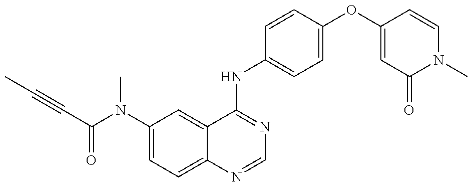 [SMILES: CC#CC(=O)N(C)c1ccc2ncnc(Nc3ccc(Oc4ccn(C)c(=O)c4)cc3)c2c1] |
| 114 | 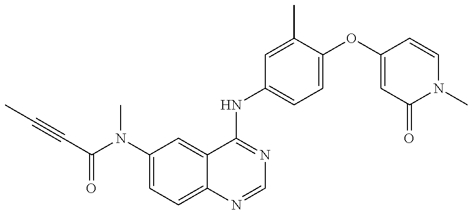 [SMILES: CC#CC(=O)N(C)c1ccc2ncnc(Nc3ccc(Oc4ccn(C)c(=O)c4)c(C)c3)c2c1] |
| 115 | 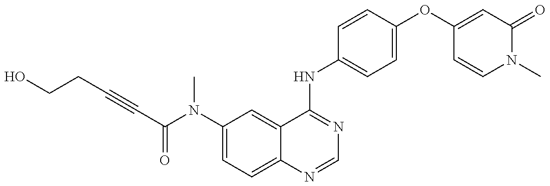 [SMILES: CN(C(=O)C#CCCO)c1ccc2ncnc(Nc3ccc(Oc4ccn(C)c(=O)c4)cc3)c2c1] |
| 116 | 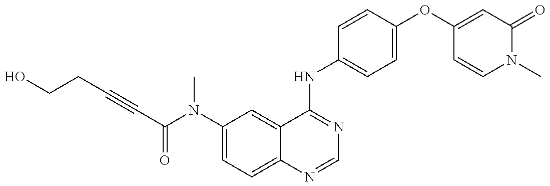 [SMILES: CN(C(=O)C#CCCO)c1ccc2ncnc(Nc3ccc(Oc4ccn(C)c(=O)c4)cc3)c2c1] |
| 117 | 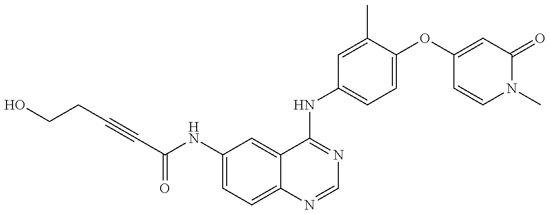 [SMILES: Cc1cc(Nc2ncnc3ccc(NC(=O)C#CCCO)cc23)ccc1Oc1ccn(C)c(=O)c1] |
| 118 | 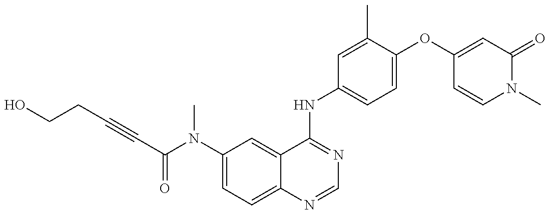 [SMILES: Cc1cc(Nc2ncnc3ccc(N(C)C(=O)C#CCCO)cc23)ccc1Oc1ccn(C)c(=O)c1] |
| 119 | 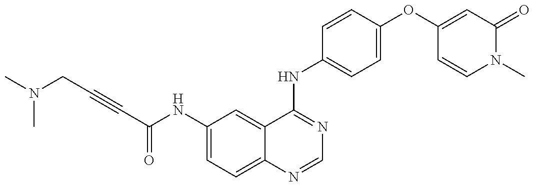 [SMILES: CN(C)CC#CC(=O)Nc1ccc2ncnc(Nc3ccc(Oc4ccn(C)c(=O)c4)cc3)c2c1] |
| 120 | 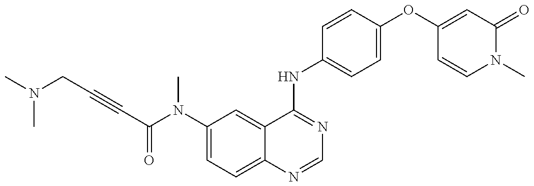 [SMILES: CN(C)CC#CC(=O)N(C)c1ccc2ncnc(Nc3ccc(Oc4ccn(C)c(=O)c4)cc3)c2c1] |
| 121 | 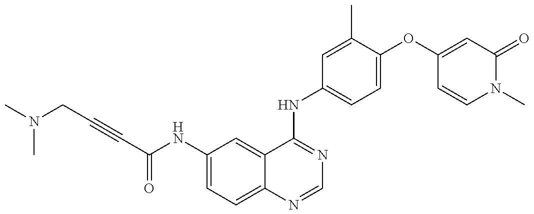 [SMILES: Cc1cc(Nc2ncnc3ccc(NC(=O)C#CCN(C)C)cc23)ccc1Oc1ccn(C)c(=O)c1] |
| 122 | 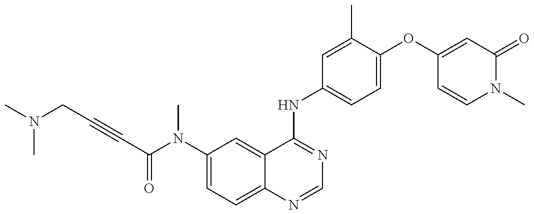 [SMILES: Cc1cc(Nc2ncnc3ccc(N(C)C(=O)C#CCN(C)C)cc23)ccc1Oc1ccn(C)c(=O)c1] |
| 123 | 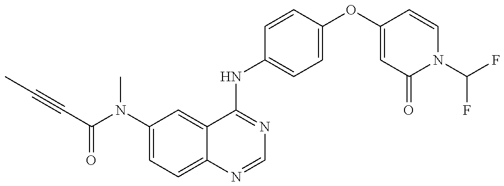 [SMILES: CC#CC(=O)N(C)c1ccc2ncnc(Nc3ccc(Oc4ccn(C(F)F)c(=O)c4)cc3)c2c1] |
| 124 | 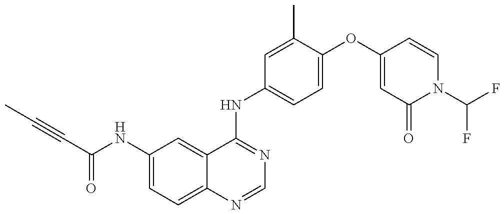 [SMILES: CC#CC(=O)Nc1ccc2ncnc(Nc3ccc(Oc4ccn(C(F)F)c(=O)c4)c(C)c3)c2c1] |
| 125 | 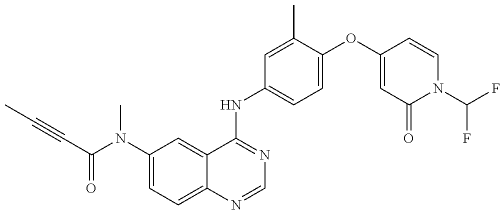 [SMILES: CC#CC(=O)N(C)c1ccc2ncnc(Nc3ccc(Oc4ccn(C(F)F)c(=O)c4)c(C)c3)c2c1] |
| 126 | 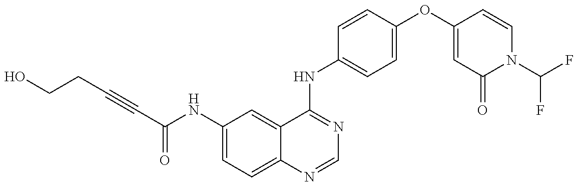 [SMILES: O=C(C#CCCO)Nc1ccc2ncnc(Nc3ccc(Oc4ccn(C(F)F)c(=O)c4)cc3)c2c1] |
| 127 | 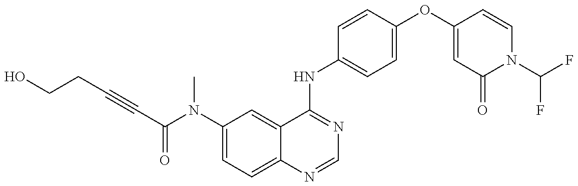 [SMILES: CN(C(=O)C#CCCO)c1ccc2ncnc(Nc3ccc(Oc4ccn(C(F)F)c(=O)c4)cc3)c2c1] |
| 128 | 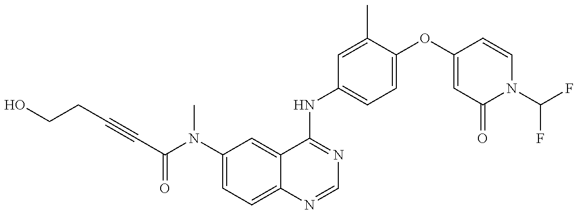 [SMILES: Cc1cc(Nc2ncnc3ccc(N(C)C(=O)C#CCCO)cc23)ccc1Oc1ccn(C(F)F)c(=O)c1] |
| 129 | 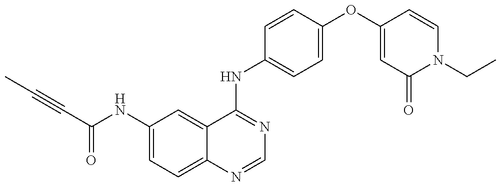 [SMILES: CC#CC(=O)Nc1ccc2ncnc(Nc3ccc(Oc4ccn(CC)c(=O)c4)cc3)c2c1] |
| 130 | 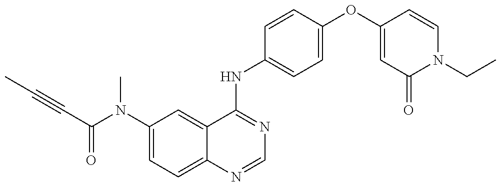 [SMILES: CC#CC(=O)N(C)c1ccc2ncnc(Nc3ccc(Oc4ccn(CC)c(=O)c4)cc3)c2c1] |
| 131 | 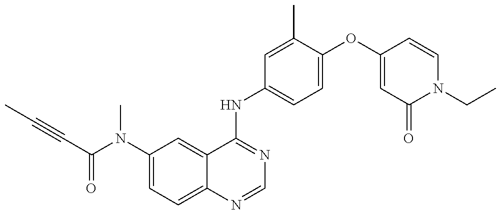 [SMILES: CC#CC(=O)N(C)c1ccc2ncnc(Nc3ccc(Oc4ccn(CC)c(=O)c4)c(C)c3)c2c1] |
| 132 | 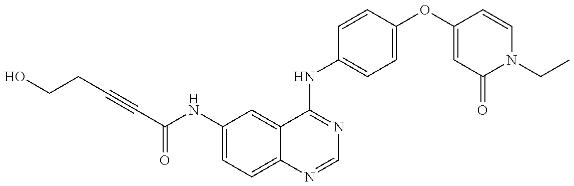 [SMILES: CCn1ccc(Oc2ccc(Nc3ncnc4ccc(NC(=O)C#CCCO)cc34)cc2)cc1=O] |
| 133 | 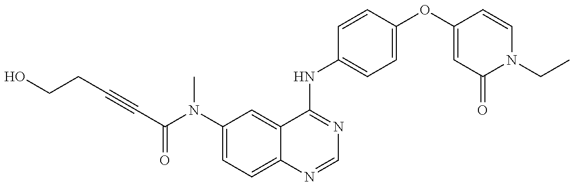 [SMILES: CCn1ccc(Oc2ccc(Nc3ncnc4ccc(N(C)C(=O)C#CCCO)cc34)cc2)cc1=O] |
| 134 | 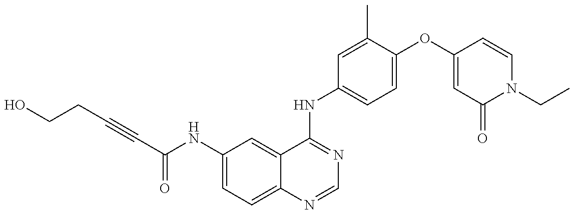 [SMILES: CCn1ccc(Oc2ccc(Nc3ncnc4ccc(NC(=O)C#CCCO)cc34)cc2C)cc1=O] |
| 135 | 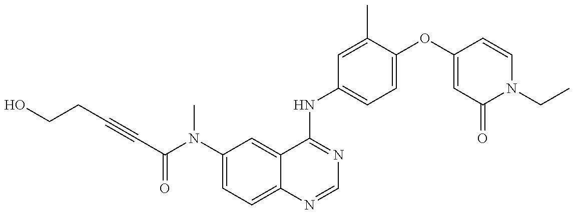 [SMILES: CCn1ccc(Oc2ccc(Nc3ncnc4ccc(N(C)C(=O)C#CCCO)cc34)cc2C)cc1=O] |
| 136 | 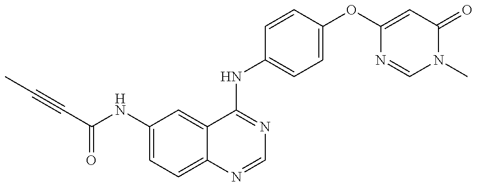 [SMILES: CC#CC(=O)Nc1ccc2ncnc(Nc3ccc(Oc4cc(=O)n(C)cn4)cc3)c2c1] |
| 137 | 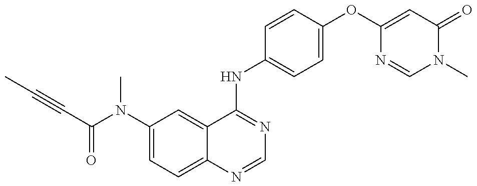 [SMILES: CC#CC(=O)N(C)c1ccc2ncnc(Nc3ccc(Oc4cc(=O)n(C)cn4)cc3)c2c1] |
| 138 | 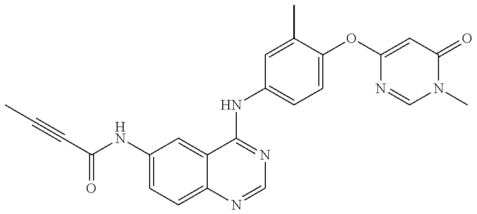 [SMILES: CC#CC(=O)Nc1ccc2ncnc(Nc3ccc(Oc4cc(=O)n(C)cn4)c(C)c3)c2c1] |
| 139 | 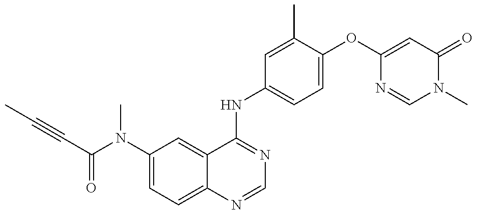 [SMILES: CC#CC(=O)N(C)c1ccc2ncnc(Nc3ccc(Oc4cc(=O)n(C)cn4)c(C)c3)c2c1] |
| 140 | 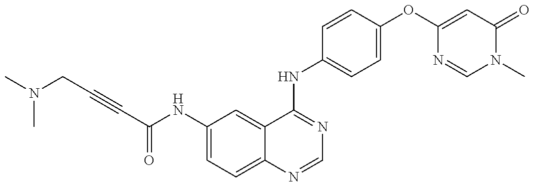 [SMILES: CN(C)CC#CC(=O)Nc1ccc2ncnc(Nc3ccc(Oc4cc(=O)n(C)cn4)cc3)c2c1] |
| 141 | 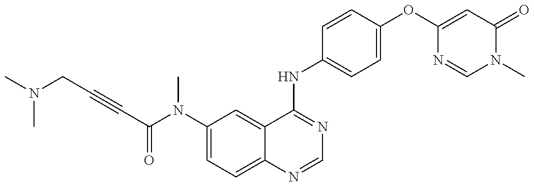 [SMILES: CN(C)CC#CC(=O)N(C)c1ccc2ncnc(Nc3ccc(Oc4cc(=O)n(C)cn4)cc3)c2c1] |
| 142 | 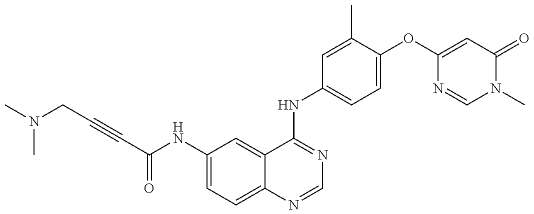 [SMILES: Cc1cc(Nc2ncnc3ccc(NC(=O)C#CCN(C)C)cc23)ccc1Oc1cc(=O)n(C)cn1] |
| 143 | 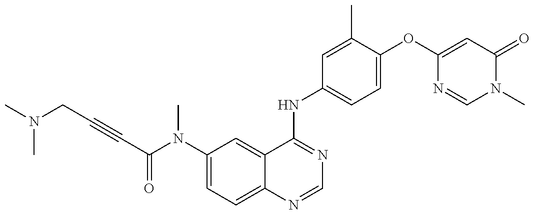 [SMILES: Cc1cc(Nc2ncnc3ccc(N(C)C(=O)C#CCN(C)C)cc23)ccc1Oc1cc(=O)n(C)cn1] |
| 144 | 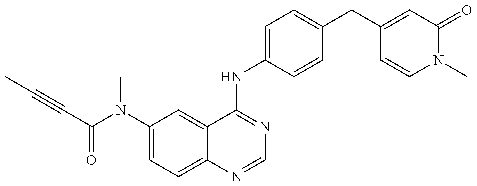 [SMILES: CC#CC(=O)N(C)c1ccc2ncnc(Nc3ccc(Cc4ccn(C)c(=O)c4)cc3)c2c1] |
| 145 | 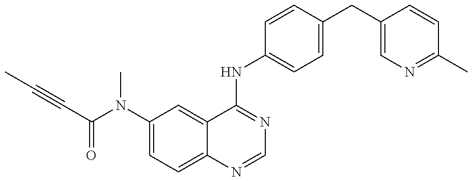 [SMILES: CC#CC(=O)N(C)c1ccc2ncnc(Nc3ccc(Cc4ccc(C)nc4)cc3)c2c1] |
| 146 | 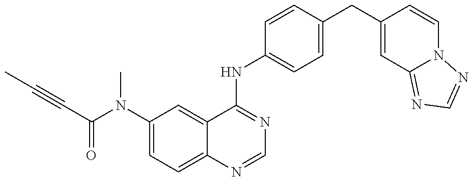 [SMILES: CC#CC(=O)N(C)c1ccc2ncnc(Nc3ccc(Cc4ccn5ncnc5c4)cc3)c2c1] |
| 147 | 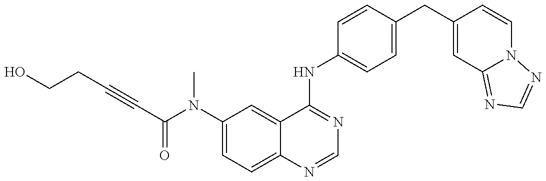 [SMILES: CN(C(=O)C#CCCO)c1ccc2ncnc(Nc3ccc(Cc4ccn5ncnc5c4)cc3)c2c1] |
| 148 | 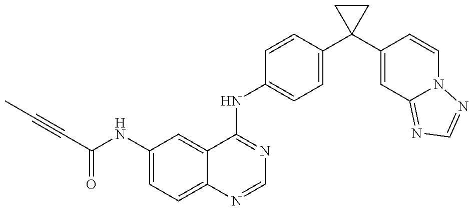 [SMILES: CC#CC(=O)Nc1ccc2ncnc(Nc3ccc(C4(c5ccn6ncnc6c5)CC4)cc3)c2c1] |
| 149 | 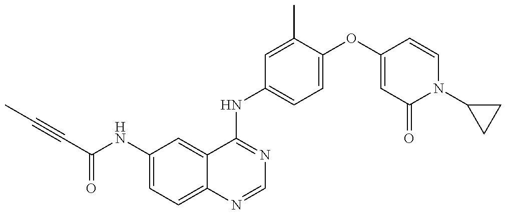 [SMILES: CC#CC(=O)Nc1ccc2ncnc(Nc3ccc(Oc4ccn(C5CC5)c(=O)c4)c(C)c3)c2c1] |
| 150 | 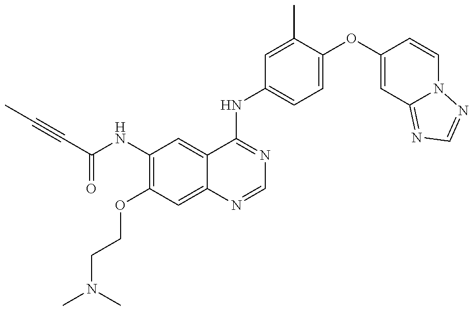 [SMILES: CC#CC(=O)Nc1cc2c(Nc3ccc(Oc4ccn5ncnc5c4)c(C)c3)ncnc2cc1OCCN(C)C] |
| 151 | 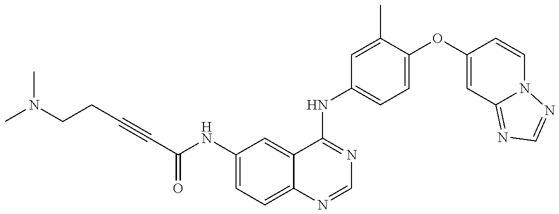 [SMILES: Cc1cc(Nc2ncnc3ccc(NC(=O)C#CCCN(C)C)cc23)ccc1Oc1ccn2ncnc2c1] |
| 152 | 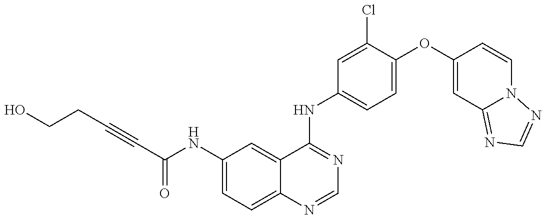 [SMILES: O=C(C#CCCO)Nc1ccc2ncnc(Nc3ccc(Oc4ccn5ncnc5c4)c(Cl)c3)c2c1] |
| 153 | 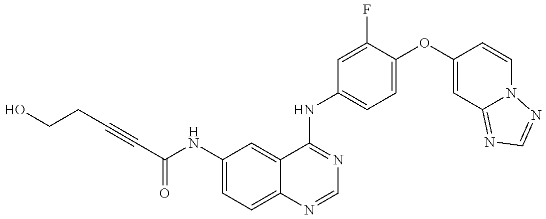 [SMILES: O=C(C#CCCO)Nc1ccc2ncnc(Nc3ccc(Oc4ccn5ncnc5c4)c(F)c3)c2c1] |
| 154 | 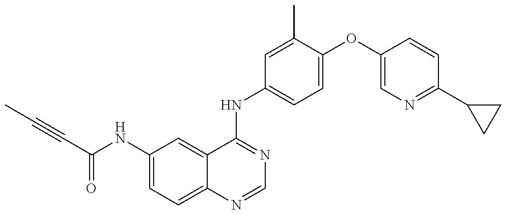 [SMILES: CC#CC(=O)Nc1ccc2ncnc(Nc3ccc(Oc4ccc(C5CC5)nc4)c(C)c3)c2c1] |
| 155 | 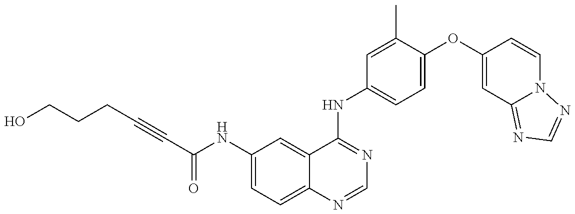 [SMILES: Cc1cc(Nc2ncnc3ccc(NC(=O)C#CCCCO)cc23)ccc1Oc1ccn2ncnc2c1] |
| 156 | 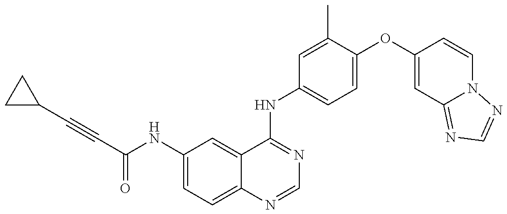 [SMILES: Cc1cc(Nc2ncnc3ccc(NC(=O)C#CC4CC4)cc23)ccc1Oc1ccn2ncnc2c1] |
| 157 | 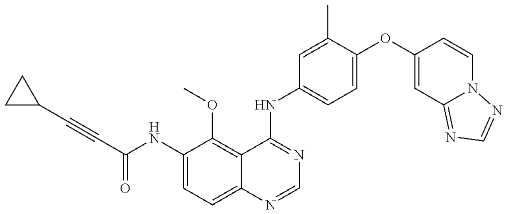 [SMILES: COc1c(NC(=O)C#CC2CC2)ccc2ncnc(Nc3ccc(Oc4ccn5ncnc5c4)c(C)c3)c12] |
| 158 | 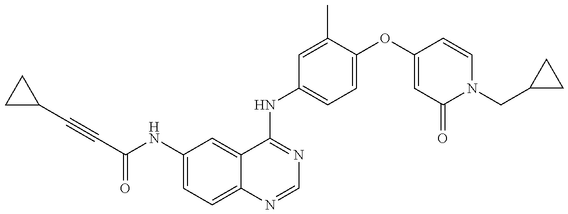 [SMILES: Cc1cc(Nc2ncnc3ccc(NC(=O)C#CC4CC4)cc23)ccc1Oc1ccn(CC2CC2)c(=O)c1] |
| 159 | 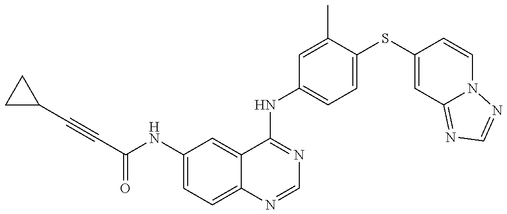 [SMILES: Cc1cc(Nc2ncnc3ccc(NC(=O)C#CC4CC4)cc23)ccc1Sc1ccn2ncnc2c1] |
| 160 | 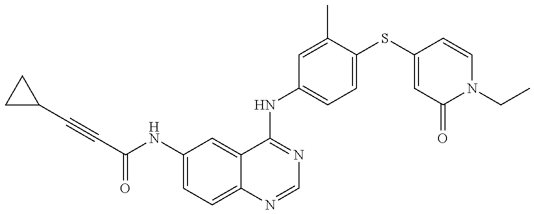 [SMILES: CCn1ccc(Sc2ccc(Nc3ncnc4ccc(NC(=O)C#CC5CC5)cc34)cc2C)cc1=O] |
| 161 | 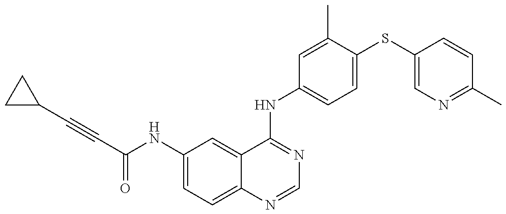 [SMILES: Cc1ccc(Sc2ccc(Nc3ncnc4ccc(NC(=O)C#CC5CC5)cc34)cc2C)cn1] |
| 162 | 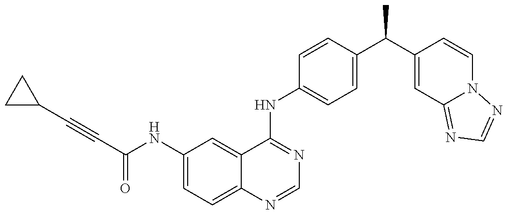 [SMILES: C[C@H](c1ccc(Nc2ncnc3ccc(NC(=O)C#CC4CC4)cc23)cc1)c1ccn2ncnc2c1] |
| 163 | 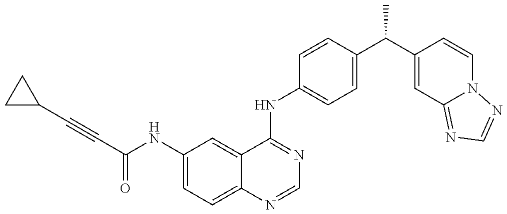 [SMILES: C[C@@H](c1ccc(Nc2ncnc3ccc(NC(=O)C#CC4CC4)cc23)cc1)c1ccn2ncnc2c1] |
| 164 | 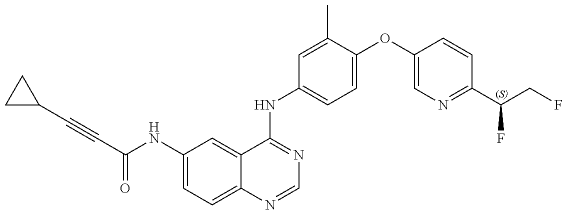 |
| 165 | 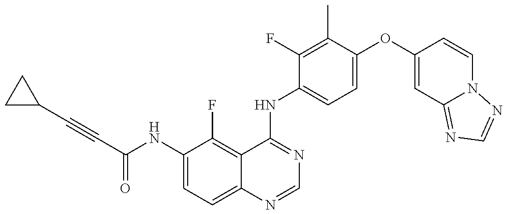 [SMILES: Cc1c(Oc2ccn3ncnc3c2)ccc(Nc2ncnc3ccc(NC(=O)C#CC4CC4)c(F)c23)c1F] |
| 166 | 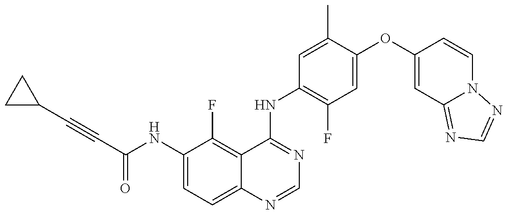 [SMILES: Cc1cc(Nc2ncnc3ccc(NC(=O)C#CC4CC4)c(F)c23)c(F)cc1Oc1ccn2ncnc2c1] |
| 167 | 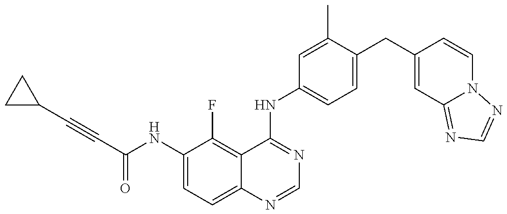 [SMILES: Cc1cc(Nc2ncnc3ccc(NC(=O)C#CC4CC4)c(F)c23)ccc1Cc1ccn2ncnc2c1] |
| 168 | 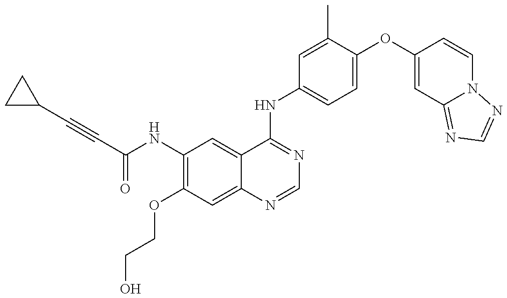 [SMILES: Cc1cc(Nc2ncnc3cc(OCCO)c(NC(=O)C#CC4CC4)cc23)ccc1Oc1ccn2ncnc2c1] |
| 169 | 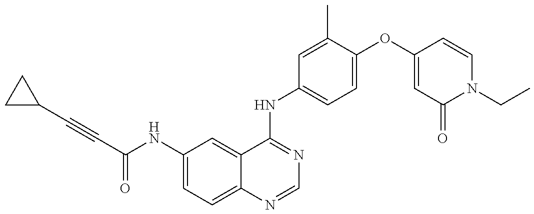 [SMILES: CCn1ccc(Oc2ccc(Nc3ncnc4ccc(NC(=O)C#CC5CC5)cc34)cc2C)cc1=O] |
| 170 | 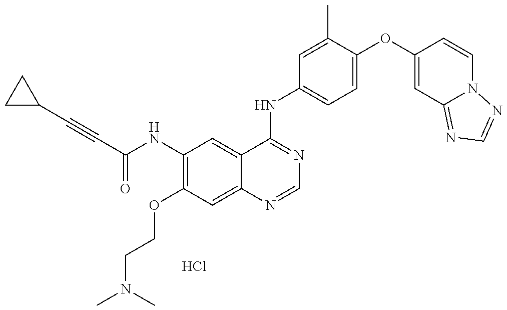 [SMILES: Cc1cc(Nc2ncnc3cc(OCCN(C)C)c(NC(=O)C#CC4CC4)cc23)ccc1Oc1ccn2ncnc2c1 ; Cl] |
| 171 | 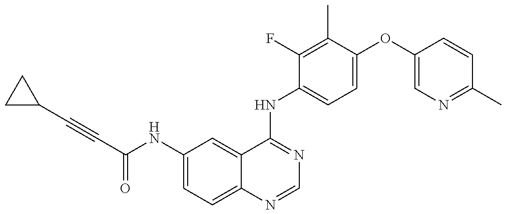 [SMILES: Cc1ccc(Oc2ccc(Nc3ncnc4ccc(NC(=O)C#CC5CC5)cc34)c(F)c2C)cn1] |
| 172 | 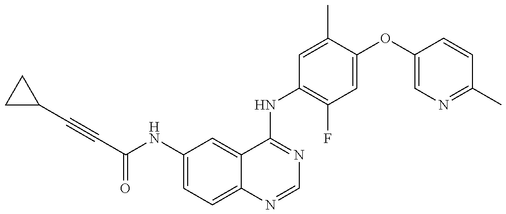 [SMILES: Cc1ccc(Oc2cc(F)c(Nc3ncnc4ccc(NC(=O)C#CC5CC5)cc34)cc2C)cn1] |
| 173 | 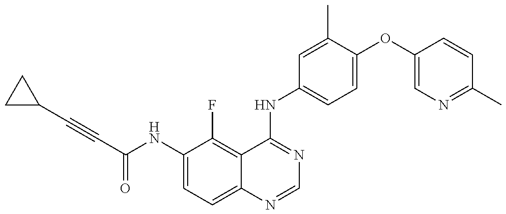 [SMILES: Cc1ccc(Oc2ccc(Nc3ncnc4ccc(NC(=O)C#CC5CC5)c(F)c34)cc2C)cn1] |
| 174 | 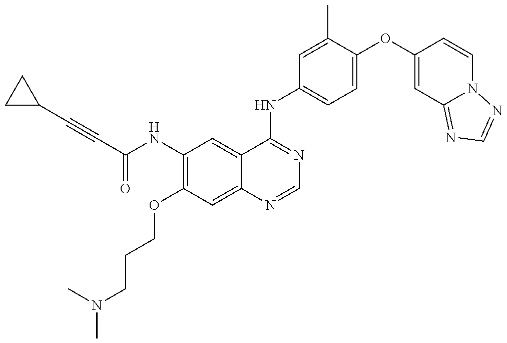 [SMILES: Cc1cc(Nc2ncnc3cc(OCCCN(C)C)c(NC(=O)C#CC4CC4)cc23)ccc1Oc1ccn2ncnc2c1] |
| 175 | 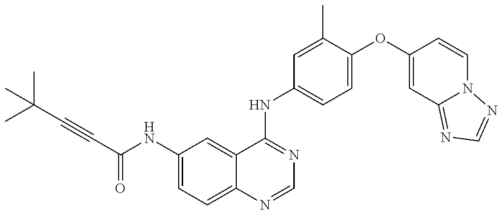 [SMILES: Cc1cc(Nc2ncnc3ccc(NC(=O)C#CC(C)(C)C)cc23)ccc1Oc1ccn2ncnc2c1] |
| 176 | 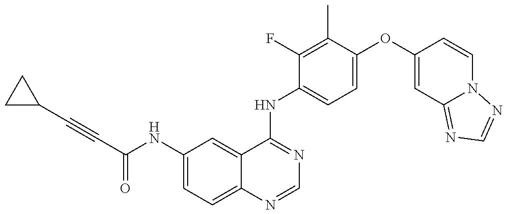 [SMILES: Cc1c(Oc2ccn3ncnc3c2)ccc(Nc2ncnc3ccc(NC(=O)C#CC4CC4)cc23)c1F] |
| 177 | 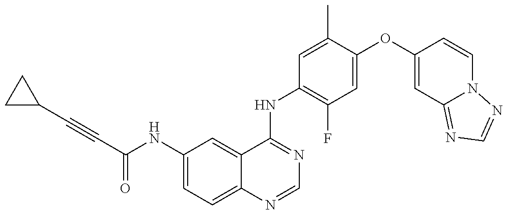 [SMILES: Cc1cc(Nc2ncnc3ccc(NC(=O)C#CC4CC4)cc23)c(F)cc1Oc1ccn2ncnc2c1] |
| 178 | 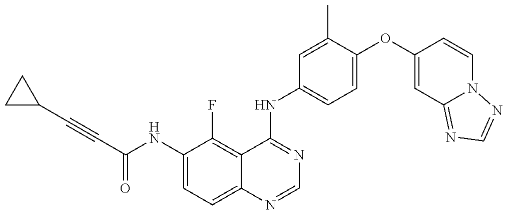 [SMILES: Cc1cc(Nc2ncnc3ccc(NC(=O)C#CC4CC4)c(F)c23)ccc1Oc1ccn2ncnc2c1] |
| 179 | 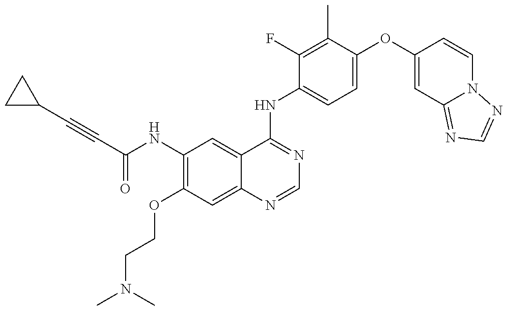 [SMILES: Cc1c(Oc2ccn3ncnc3c2)ccc(Nc2ncnc3cc(OCCN(C)C)c(NC(=O)C#CC4CC4)cc23)c1F] |
| 180 | 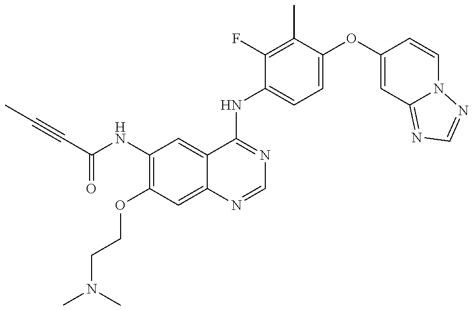 [SMILES: CC#CC(=O)Nc1cc2c(Nc3ccc(Oc4ccn5ncnc5c4)c(C)c3F)ncnc2cc1OCCN(C)C] |
| 181 | 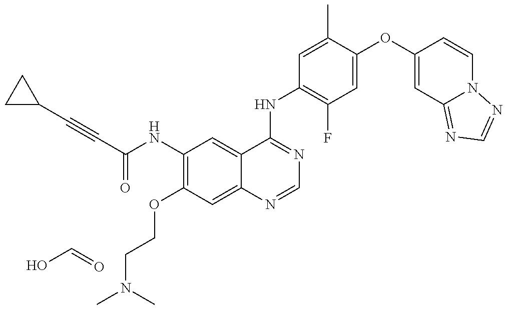 |
| 182 | 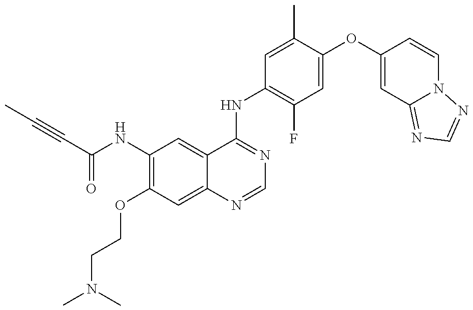 [SMILES: CC#CC(=O)Nc1cc2c(Nc3cc(C)c(Oc4ccn5ncnc5c4)cc3F)ncnc2cc1OCCN(C)C] |
| 183 | 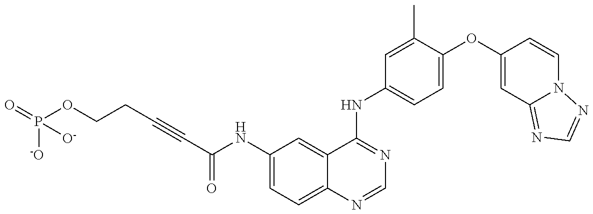 |
| 184 | 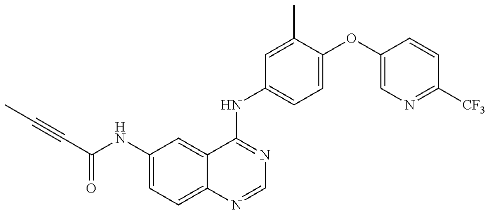 [SMILES: CC#CC(=O)Nc1ccc2ncnc(Nc3ccc(Oc4ccc(C(F)(F)F)nc4)c(C)c3)c2c1] |
| 185 | 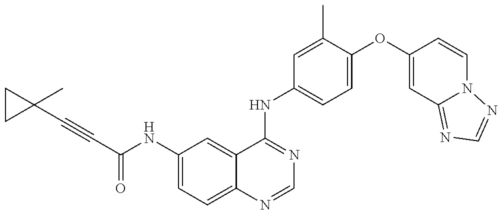 [SMILES: Cc1cc(Nc2ncnc3ccc(NC(=O)C#CC4(C)CC4)cc23)ccc1Oc1ccn2ncnc2c1] |
| 186 | 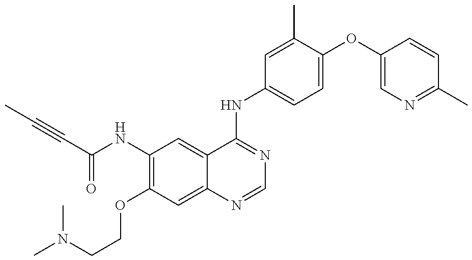 [SMILES: CC#CC(=O)Nc1cc2c(Nc3ccc(Oc4ccc(C)nc4)c(C)c3)ncnc2cc1OCCN(C)C] |
| 187 | 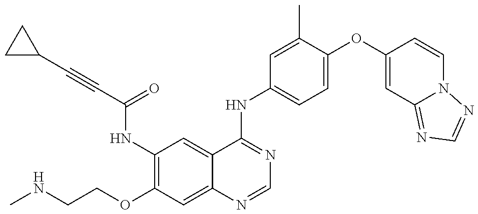 [SMILES: CNCCOc1cc2ncnc(Nc3ccc(Oc4ccn5ncnc5c4)c(C)c3)c2cc1NC(=O)C#CC1CC1] |
| 188 | 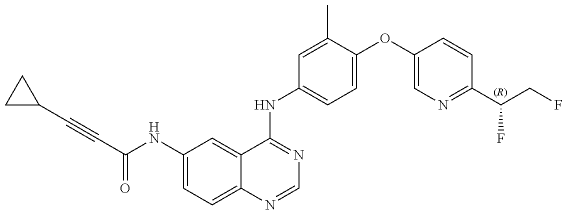 |
| 189 | 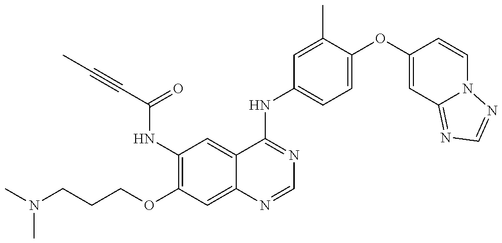 [SMILES: CC#CC(=O)Nc1cc2c(Nc3ccc(Oc4ccn5ncnc5c4)c(C)c3)ncnc2cc1OCCCN(C)C] |
| 190 | 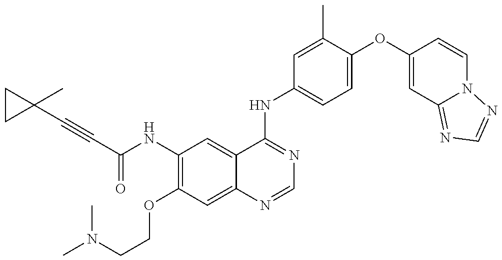 [SMILES: Cc1cc(Nc2ncnc3cc(OCCN(C)C)c(NC(=O)C#CC4(C)CC4)cc23)ccc1Oc1ccn2ncnc2c1] |
| 191 | 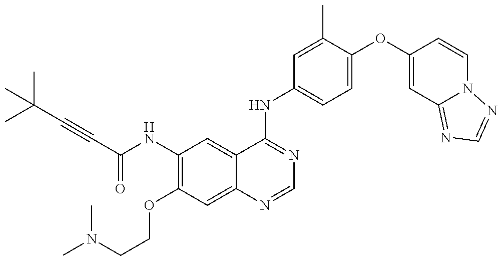 [SMILES: Cc1cc(Nc2ncnc3cc(OCCN(C)C)c(NC(=O)C#CC(C)(C)C)cc23)ccc1Oc1ccn2ncnc2c1] |
| 192 | 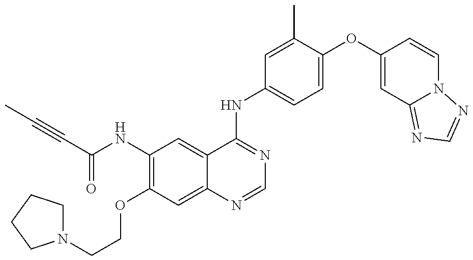 [SMILES: CC#CC(=O)Nc1cc2c(Nc3ccc(Oc4ccn5ncnc5c4)c(C)c3)ncnc2cc1OCCN1CCCC1] |
| 193 | 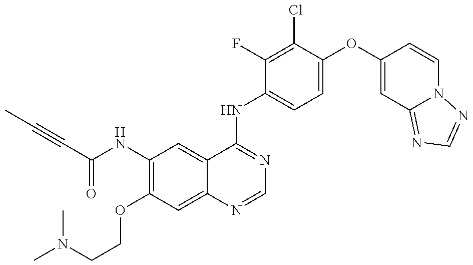 [SMILES: CC#CC(=O)Nc1cc2c(Nc3ccc(Oc4ccn5ncnc5c4)c(Cl)c3F)ncnc2cc1OCCN(C)C] |
| 194 | 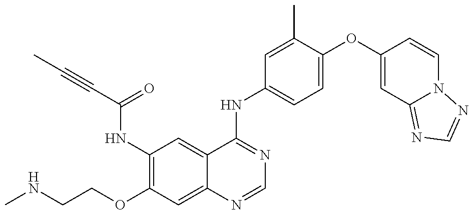 [SMILES: CC#CC(=O)Nc1cc2c(Nc3ccc(Oc4ccn5ncnc5c4)c(C)c3)ncnc2cc1OCCNC] |
| 195 | 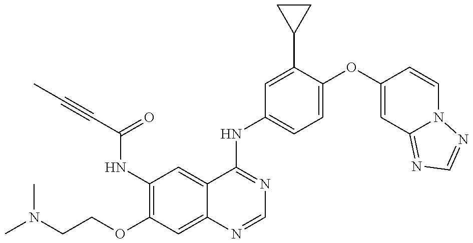 [SMILES: CC#CC(=O)Nc1cc2c(Nc3ccc(Oc4ccn5ncnc5c4)c(C4CC4)c3)ncnc2cc1OCCN(C)C] |
| 196 | 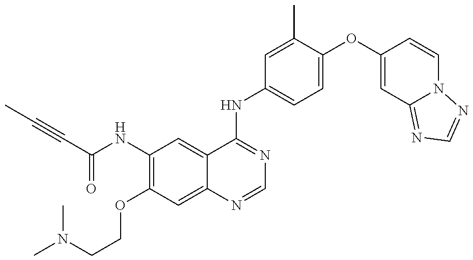 [SMILES: CC#CC(=O)Nc1cc2c(Nc3ccc(Oc4ccn5ncnc5c4)c(C)c3)ncnc2cc1OCCN(C)C] |
| 197 | 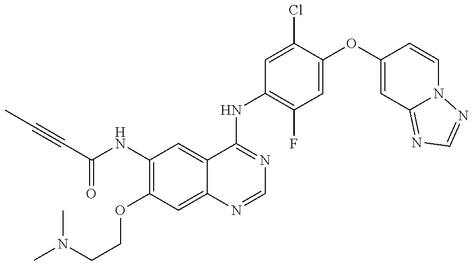 [SMILES: CC#CC(=O)Nc1cc2c(Nc3cc(Cl)c(Oc4ccn5ncnc5c4)cc3F)ncnc2cc1OCCN(C)C] |
| 198 | 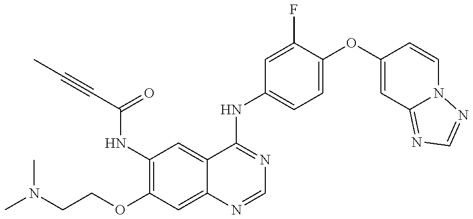 [SMILES: CC#CC(=O)Nc1cc2c(Nc3ccc(Oc4ccn5ncnc5c4)c(F)c3)ncnc2cc1OCCN(C)C] |
| 199 | 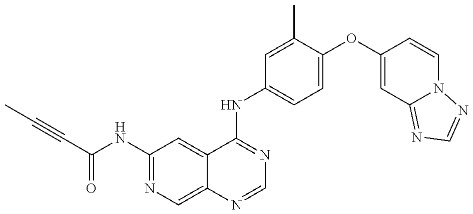 [SMILES: CC#CC(=O)Nc1cc2c(Nc3ccc(Oc4ccn5ncnc5c4)c(C)c3)ncnc2cn1] |
| 200 | 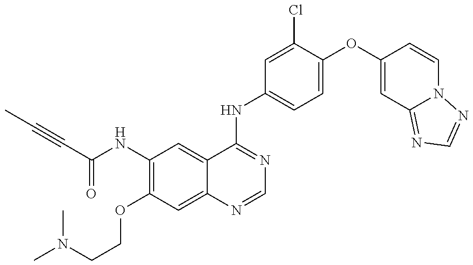 [SMILES: CC#CC(=O)Nc1cc2c(Nc3ccc(Oc4ccn5ncnc5c4)c(Cl)c3)ncnc2cc1OCCN(C)C] |
| 201 | 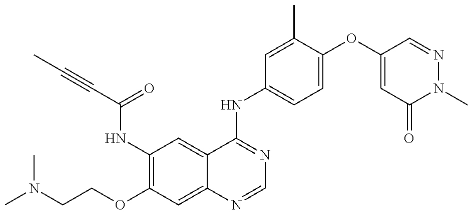 |
| 202 | 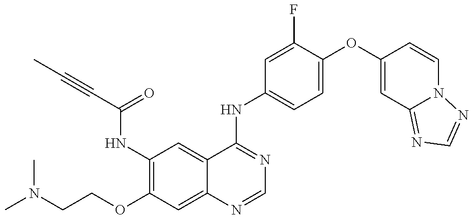 [SMILES: CC#CC(=O)Nc1cc2c(Nc3ccc(Oc4ccn5ncnc5c4)c(F)c3)ncnc2cc1OCCN(C)C] |
| 203 | 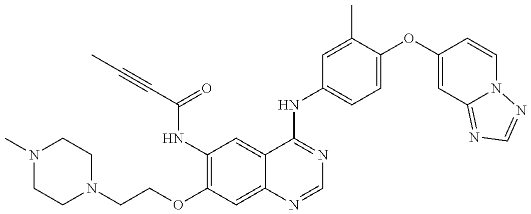 [SMILES: CC#CC(=O)Nc1cc2c(Nc3ccc(Oc4ccn5ncnc5c4)c(C)c3)ncnc2cc1OCCN1CCN(C)CC1] |
| 204 | 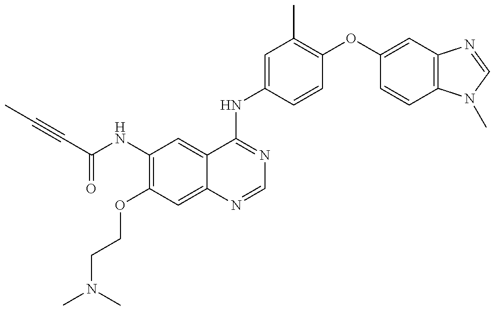 [SMILES: CC#CC(=O)Nc1cc2c(Nc3ccc(Oc4ccc5c(c4)ncn5C)c(C)c3)ncnc2cc1OCCN(C)C] |
| 205 | 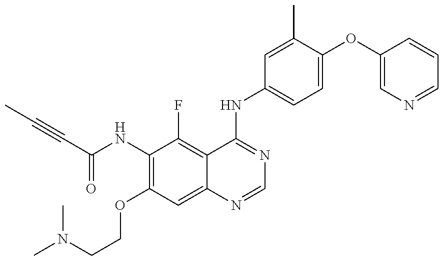 [SMILES: CC#CC(=O)Nc1c(OCCN(C)C)cc2ncnc(Nc3ccc(Oc4cccnc4)c(C)c3)c2c1F] |
| 206 | 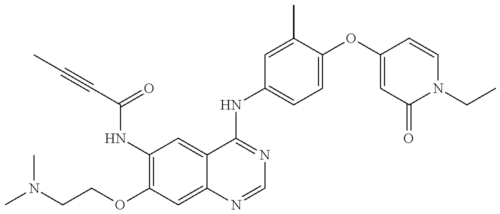 [SMILES: CC#CC(=O)Nc1cc2c(Nc3ccc(Oc4ccn(CC)c(=O)c4)c(C)c3)ncnc2cc1OCCN(C)C] |
| 207 | 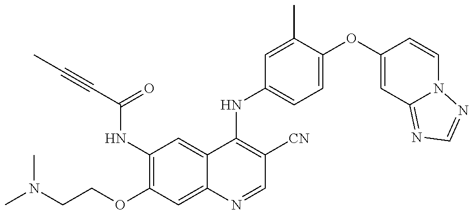 [SMILES: CC#CC(=O)Nc1cc2c(Nc3ccc(Oc4ccn5ncnc5c4)c(C)c3)c(C#N)cnc2cc1OCCN(C)C] |
| 208 | 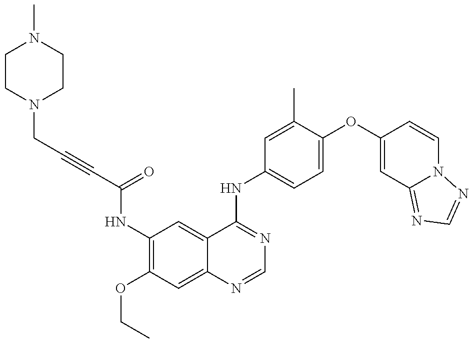 [SMILES: CCOc1cc2ncnc(Nc3ccc(Oc4ccn5ncnc5c4)c(C)c3)c2cc1NC(=O)C#CCN1CCN(C)CC1] |
| 209 | 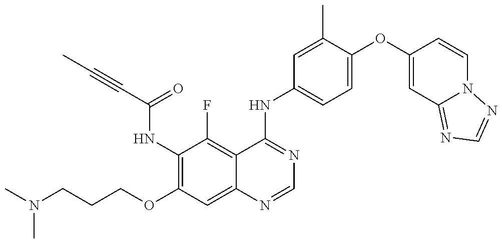 [SMILES: CC#CC(=O)Nc1c(OCCCN(C)C)cc2ncnc(Nc3ccc(Oc4ccn5ncnc5c4)c(C)c3)c2c1F] |
| 210 | 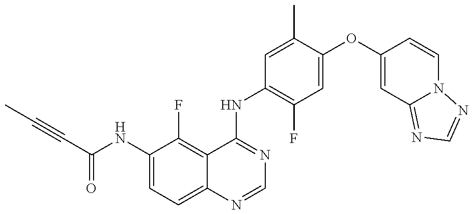 [SMILES: CC#CC(=O)Nc1ccc2ncnc(Nc3cc(C)c(Oc4ccn5ncnc5c4)cc3F)c2c1F] |
| 211 | 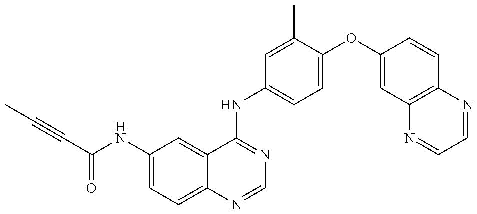 [SMILES: CC#CC(=O)Nc1ccc2ncnc(Nc3ccc(Oc4ccc5nccnc5c4)c(C)c3)c2c1] |
| 212 | 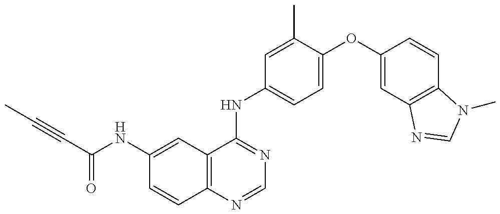 [SMILES: CC#CC(=O)Nc1ccc2ncnc(Nc3ccc(Oc4ccc5c(c4)ncn5C)c(C)c3)c2c1] |
| 213 | 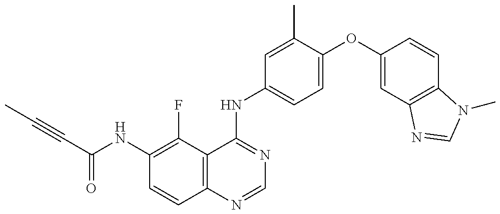 [SMILES: CC#CC(=O)Nc1ccc2ncnc(Nc3ccc(Oc4ccc5c(c4)ncn5C)c(C)c3)c2c1F] |

### III. Pharmaceutical Compositions and Formulations

Any of the compounds described herein may be formulated as a pharmaceutically acceptable composition.

Pharmaceutical compositions of any of the compounds detailed herein are embraced by this disclosure. Thus, the present disclosure includes pharmaceutical compositions comprising a compound as detailed herein, or a pharmaceutically acceptable salt, solvate, hydrate, or co-crystal thereof, or a mixture of any of the foregoing, and a pharmaceutically acceptable carrier or excipient. In one aspect, the pharmaceutically acceptable salt is an acid addition salt, such as a salt formed with an inorganic or organic acid. Pharmaceutical compositions may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound, or a pharmaceutically acceptable salt, solvate, hydrate, or co-crystal thereof, or a mixture of any of the foregoing, as detailed herein are provided, such as compositions of substantially pure compounds. In some embodiments, a composition containing a compound, or a pharmaceutically acceptable salt, solvate, hydrate, or co-crystal thereof, or a mixture of any of the foregoing, as detailed herein is in substantially pure form. In one variation, “substantially pure” intends a composition that contains no more than 35% impurity, wherein the impurity denotes a compound other than the compound comprising the majority of the composition or a salt thereof. For example, a composition of a substantially pure compound selected from a compound of Table 1 intends a composition that contains no more than 35% impurity, wherein the impurity denotes a compound other than the compound of Table 1. In one variation, a composition of substantially pure compound, or a pharmaceutically acceptable salt, solvate, hydrate, or co-crystal thereof, or a mixture of any of the foregoing, is provided wherein the composition contains no more than 25% impurity. In another variation, a composition of substantially pure compound, or a pharmaceutically acceptable salt, solvate, hydrate, or co-crystal thereof, or a mixture of any of the foregoing, is provided wherein the composition contains or no more than 20% impurity. In still another variation, a composition of substantially pure compound, or a pharmaceutically acceptable salt, solvate, hydrate, or co-crystal thereof, or a mixture of any of the foregoing, is provided wherein the composition contains or no more than 10% impurity. In a further variation, a composition of substantially pure compound, or a pharmaceutically acceptable salt, solvate, hydrate, or co-crystal thereof, or a mixture of any of the foregoing, is provided wherein the composition contains no more than 5% impurity. In another variation, a composition of substantially pure compound, or a pharmaceutically acceptable salt, solvate, hydrate, or co-crystal thereof, or a mixture of any of the foregoing, is provided wherein the composition contains no more than 3% impurity. In still another variation, a composition of substantially pure compound, or a pharmaceutically acceptable salt, solvate, hydrate, or co-crystal thereof, or a mixture of any of the foregoing, is provided wherein the composition contains no more than 1% impurity. In a further variation, a composition of substantially pure compound, or a pharmaceutically acceptable salt, solvate, hydrate, or co-crystal thereof, or a mixture of any of the foregoing, is provided wherein the composition contains no more than 0.5% impurity. In yet other variations, a composition of substantially pure compound means that the composition contains no more than 15%, no more than 10%, no more than 5%, no more than 3%, or no more than 1% impurity, which impurity may be the compound in a different stereochemical form. For instance, and without limitation, a composition of substantially pure (S) compound means that the composition contains no more than 15% or no more than 10% or no more than 5% or no more than 3% or no more than 1% of the (R) form of the compound.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

A compound detailed herein, or a pharmaceutically acceptable salt, solvate, hydrate, or co-crystal thereof, or a mixture of any of the foregoing, may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound, or a pharmaceutically acceptable salt, solvate, hydrate, or co-crystal thereof, or a mixture of any of the foregoing, may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

A compound detailed herein, or a pharmaceutically acceptable salt, solvate, hydrate, or co-crystal thereof, or a mixture of any of the foregoing, can be used in the preparation of a formulation, such as a pharmaceutical formulation, by combining the compound or compounds, or a pharmaceutically acceptable salt, solvate, hydrate, or co-crystal thereof, or a mixture of any of the foregoing, with a pharmaceutically acceptable carrier. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, Pa., 20th ed. (2000), which is incorporated herein by reference.

A compound detailed herein, or a pharmaceutically acceptable salt, solvate, hydrate, or co-crystal thereof, or a mixture of any of the foregoing, may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds, or a pharmaceutically acceptable salt, solvate, hydrate, or co-crystal thereof, or a mixture of any of the foregoing, described herein can be formulated in a tablet in any dosage form described, for example, a compound as described herein, or a pharmaceutically acceptable salt, solvate, hydrate, or co-crystal thereof, or a mixture of any of the foregoing, can be formulated as a 10 mg tablet.

Compositions comprising a compound, or a pharmaceutically acceptable salt, solvate, hydrate, or co-crystal thereof, or a mixture of any of the foregoing, provided herein are also described. In one variation, the composition comprises a compound, or a pharmaceutically acceptable salt, solvate, hydrate, or co-crystal thereof, or a mixture of any of the foregoing, and a pharmaceutically acceptable carrier or excipient. In another variation, a composition of substantially pure compound, or a pharmaceutically acceptable salt, solvate, hydrate, or co-crystal thereof, or a mixture of any of the foregoing, is provided. In some embodiments, the composition is for use as a human or veterinary medicament. In some embodiments, the composition is for use in a method described herein. In some embodiments, the composition is for use in the treatment of a disease or disorder described herein.

Compositions formulated for co-administration of a compound provided herein and one or more additional pharmaceutical agents are also described. The co-administration can be simultaneous or sequential in any order. A compound provided herein may be formulated for co-administration with the one or more additional pharmaceutical agents in the same dosage form (e.g., single tablet or single i.v.) or separate dosage forms (e.g., two separate tablets, two separate i.v., or one tablet and one i.v.). Furthermore, co-administration can be, for example, 1) concurrent delivery, through the same route of delivery (e.g., tablet or i.v.), 2) sequential delivery on the same day, through the same route or different routes of delivery, or 3) delivery on different days, through the same route or different routes of delivery.

### IV. Methods of Use

Compounds and compositions detailed herein, such as a pharmaceutical composition containing a compound of formula (I) or formula (II) or any variation thereof provided herein, or a pharmaceutically acceptable salt, solvate, hydrate, or co-crystal thereof, or a mixture of any of the foregoing, and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

In one aspect, provided herein is a method of inhibiting kinase activity of a human receptor tyrosine kinase ErbB2 or a mutant form of human ErbB2, comprising contacting the ErbB2 or the mutant form with a therapeutically effective amount of a compound or composition provided herein. In some embodiments, provided herein is a method of inhibiting kinase activity of a human receptor tyrosine kinase ErbB2 or a mutant form of human ErbB2 in a cell, comprising administering an effective amount of a compound or composition of the disclosure to the cell. In some embodiments, provided herein is a method of inhibiting kinase activity of a human receptor tyrosine kinase ErbB2 or a mutant form of human ErbB2 in an individual in need thereof, comprising administering an effective amount of a compound or composition of the disclosure to the individual.

In some embodiments, the mutant form of human ErbB2 comprises a mutation in Exon 20 that introduces certain amino acid deletions and/or insertions selected from the group consisting of: A775_A776insYVMA, G778_P780insGSP, G776delinsVC, P780_Y781insGSP, M774delinsWLV, A775_G776insSVMA, A775_G776insI, G776delinsLC, G778_S779InsCPG, V777_G778insGSP. In other embodiments, the mutant form of human ErbB2 comprises one or more mutations that introduce certain amino acid substitutions selected from the group consisting of: P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, N1219S, and A1232fs. In some embodiments, the mutant form of human ErbB2 comprises one or more mutations that introduce (a) an amino acid substitution selected from the group consisting of: P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, and N1219S; or (b) a frameshift at A1232.

In some variations, the compounds provided herein are selective for inhibiting human receptor tyrosine kinase ErbB2. As such, in some embodiments, provided herein is a method of selectively inhibiting human receptor tyrosine kinase ErbB2, as compared to other receptor tyrosine kinases, including but not limited to ErbB1 (EGFR), ErbB3, or ErbB4.

The compounds and compositions described herein may be used in a method of treating a disease or disorder in an individual, wherein the individual has cells or cell tissue having increased ErbB2 kinase activity, for example, as compared to the ErbB2 kinase activity in a corresponding cell type or cell tissue from a healthy individual. In some embodiments, the compound or composition is administered according to a dosage described herein.

In some embodiments, provided herein is a method for treating a disease or disorder in an individual, wherein the individual has cells or cell tissue having increased ErbB2 kinase activity, comprising administering to an individual in need of treatment a therapeutically effective amount of a compound of formula (I) or formula (II) or any variation thereof, or a pharmaceutically acceptable salt, solvate, hydrate, or co-crystal thereof, or a mixture of any of the foregoing, or a therapeutically effective amount of a composition as described herein. In some embodiments, the disease or disorder is cancer. In some embodiments, the disease or disorder is lung cancer, glioma, head and/or neck cancer, salivary gland cancer, breast cancer, esophageal cancer, liver cancer, stomach (gastric) cancer, uterine cancer, cervical cancer, biliary tract cancer, pancreatic cancer, colorectal cancer, renal cancer, bladder cancer, or prostate cancer. In some embodiments, the cancer is non-small cell lung cancer. In some embodiments, the individual has received at least one, at least two or at least three prior therapies for the cancer. In certain embodiments, the one or more prior therapies are selected from the group consisting of lapatinib, neratinib, afatinib, pyrotinib, poziotinib, TAK-788 and tucatinib.

In some embodiments, the disease or disorder is refractory or resistant to first-line treatment, second-line treatment, and/or third-line treatment. In certain embodiments, the condition having increased activation of ErbB2 kinase activity is refractory or resistant to treatment with one or more tyrosine kinase inhibitors selected from the group consisting of lapatinib, neratinib, afatinib, pyrotinib, poziotinib, TAK-788, and tucatinib.

Resistant subtypes of tyrosine kinase-mediated diseases or disorders may be associated with any number of ErbB2 independent resistance mechanisms. In some embodiments wherein the disease or disorder in the individual having cells or cell tissue with increased ErbB2 kinase activity is refractory to treatment, the disease or disorder is characterized as being associated with one or more ErbB2 dependent resistance mechanisms. ErbB2-dependent resistance mechanisms include, but are not limited to, one or more mutations in Exon 20 of ErbB2 or other disease-associated point mutations. The one or more mutations of ErbB2 introduce certain amino acid deletions and/or insertions, for example, A775_A776insYVMA, G778_P780insGSP, G776delinsVC, P780_Y781insGSP, M774delinsWLV, A775_G776insSVMA, A775_G776insI, G776delinsLC, G778_S779InsCPG, and/or V777_G778insGSP. In other variations, the mutations introduce certain amino acid substitutions, for example, P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, N1219S, and/or A1232fs. In some variations, the mutations introduce certain (a) amino acid substitutions, for example, P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, and N1219S, and/or (b) frameshifts, such as a frameshift at A1232. In some variations, the refractory disease or disorder in an individual having increased activation of the ErbB2 kinase activity is associated with one or more mutations in Exon 20 of the ErbB2. In certain variations, the one or more mutations in Exon 20 of the ErbB2 that introduce certain amino acid deletions and/or insertions selected from the group consisting of: A775_A776insYVMA, G778_P780insGSP, G776delinsVC, P780_Y781insGSP, M774delinsWLV, A775_G776insSVMA, A775_G776insI, G776delinsLC, G778_S779InsCPG, and V777_G778insGSP. In other variations, the refractory disease or disorder in an individual having increased activation of the ErbB2 kinase activity is associated with one or more disease-associated point mutations. In certain variations, the one or more point mutations introduce certain amino acid substitutions selected from the group consisting of: P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, N1219S, and A1232fs. In certain variations, the one or more point mutations introduce (a) an amino acid substitution selected from the group consisting of: P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, and N1219S, or (b) a frameshift at A1232.

In some embodiments, provided is a method for treating cancer in an individual in need thereof, comprising administering to the individual a therapeutically effective amount of a compound of formula (I) or formula (II), or any variation thereof as described herein, or a therapeutically effective amount of a composition as described herein. In some embodiments, the cancer comprises cells or cell tissue having increased ErbB2 kinase activity, for example, as compared to the ErbB2 kinase activity in a corresponding cell type or cell tissue from a healthy individual. In some embodiments, the cancer comprises cells or cell tissue having one or more mutations in Exon 20 of the ErbB2. In certain embodiments, the one or more mutations in Exon 20 of the ErbB2 introduce certain amino acid deletions and/or insertions selected from the group consisting of A775_A776insYVMA, G778_P780insGSP, G776delinsVC, P780_Y781insGSP, M774delinsWLV, A775_G776insSVMA, A775_G776insI, G776delinsLC, G778_S779InsCPG, and V777_G778insGSP. In some embodiments, the cancer comprises cells or cell tissue comprising one or more disease-associated point mutations. In certain embodiments, the one or more point mutations introduce certain amino acid substitutions selected from the group consisting of: P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, N1219S, and A1232fs. In certain embodiments, the one or more point mutations introduce (a) an amino acid substitution selected from the group consisting of: P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, and N1219S; or (b) a frameshift at A1232. In some embodiments, the disease or disorder is lung cancer, glioma, head and/or neck cancer, salivary gland cancer, breast cancer, esophageal cancer, liver cancer, stomach (gastric) cancer, uterine cancer, cervical cancer, biliary tract cancer, pancreatic cancer, colorectal cancer, renal cancer, bladder cancer, or prostate cancer. In some embodiments, the cancer is non-small cell lung cancer.

In one aspect, provided herein is a method of treating cancer in an individual in need thereof, wherein modulation of ErbB2 kinase activity inhibits or ameliorates the pathology and/or symptomology of the cancer, comprising administering to the individual a therapeutically effective amount of a compound or composition provided herein. In one embodiment, provided herein is a method of treating cancer, wherein modulation of ErbB2 kinase activity inhibits the pathology and/or symptomology of the cancer, in an individual, comprising administering to the individual a therapeutically effective amount of a compound or composition provided herein. In one embodiment, provided herein is a method of treating a cancer, wherein modulation of ErbB2 kinase activity ameliorates the pathology and/or symptomology of the cancer, in an individual, comprising administering to the individual a therapeutically effective amount of a compound or composition provided herein.

In another aspect, provided herein is a method of preventing cancer, wherein modulation of ErbB2 kinase activity prevents the pathology and/or symptomology of the cancer, in an individual, comprising administering to the individual a therapeutically effective amount of a compound or composition provided herein. In another aspect, provided herein is a method of delaying the onset and/or development of a cancer in an individual (such as a human) who is at risk for developing the cancer, e.g., an individual who has cells or cell tissue having increased ErbB2 kinase activity. It is appreciated that delayed development may encompass prevention in the event the individual does not develop the cancer.

In one aspect, provided herein is a method of delaying the onset and/or development of cancer in an individual having cells or cell tissue having increased ErbB2 kinase activity in need thereof, comprising administering to the individual a therapeutically effective amount of a compound or composition provided herein. In some embodiments, the cancer is lung cancer, glioma, head and/or neck cancer, salivary gland cancer, breast cancer, esophageal cancer, liver cancer, stomach (gastric) cancer, uterine cancer, cervical cancer, biliary tract cancer, pancreatic cancer, colorectal cancer, renal cancer, bladder cancer, or prostate cancer. In some embodiments, the cancer is non-small cell lung cancer.

In one aspect, provided herein is a compound of formula (I) or formula (II) or any variation thereof, or a pharmaceutically acceptable salt, solvate, hydrate, or co-crystal thereof, or a mixture of any of the foregoing, for use in therapy. In some embodiments, provided herein is a compound of formula (I) or formula (II) or any variation thereof, or a pharmaceutically acceptable salt, solvate, hydrate, or co-crystal thereof, or a mixture of any of the foregoing, or pharmaceutical composition comprising such compound, for use in the treatment of cancer. In some embodiments, provided is a compound of formula (I) or formula (II) or any variation thereof, or a pharmaceutically acceptable salt, solvate, hydrate, or co-crystal thereof, or a mixture of any of the foregoing, or a pharmaceutical composition comprising such compound, for use in the treatment of cancer. In some embodiments, provided is a compound of formula (I) or formula (II) or any variation thereof, or a pharmaceutically acceptable salt, solvate, hydrate, or co-crystal thereof, or a mixture of any of the foregoing, or a pharmaceutical composition comprising such compound, for use in the treatment of cancer, wherein the cancer comprises cells or cell tissue having increased activation of ErbB2 kinase activity. In some embodiments, provided is a compound of formula (I) or formula (II) or any variation thereof, or a pharmaceutically acceptable salt, solvate, hydrate, or co-crystal thereof, or a mixture of any of the foregoing, or a pharmaceutical composition comprising such compound, for use in the treatment of cancer, wherein the cancer comprises cells or cell tissue having one or more mutations in Exon 20 of the ErbB2. In some embodiments, provided is a compound of formula (I) or formula (II) or any variation thereof, or a pharmaceutically acceptable salt, solvate, hydrate, or co-crystal thereof, or a mixture of any of the foregoing, or a pharmaceutical composition comprising such compound, for use in the treatment of cancer, wherein the cancer cells comprise one or more genetic alterations in Exon 20 of the ErbB2 that introduce certain amino acid deletions and/or insertions selected from the group consisting of A775_A776insYVMA, G778_P780insGSP, G776delinsVC, P780_Y781insGSP, M774delinsWLV, A775_G776insSVMA, A775_G776insI, G776delinsLC, G778_S779InsCPG, and V777_G778insGSP. In some embodiments, provided is a compound of formula (I) or formula (II) or any variation thereof, or a pharmaceutically acceptable salt, solvate, hydrate, or co-crystal thereof, or a mixture of any of the foregoing, or a pharmaceutical composition comprising such compound, for use in the treatment of cancer, wherein the cancer comprises cells or cell tissue having one or more disease-associated point mutations in ErbB2. In certain embodiments, the cancer comprises cells or cell tissue having one or more point mutations that introduce certain amino acid substitutions selected from the group consisting of P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, N1219S, and A1232fs. In certain embodiments, the cancer comprises cells or cell tissue having one or more point mutations that introduce (a) an amino acid substitution selected from the group consisting of P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, and N1219S; or (b) a frameshift at A1232. In some embodiments, provided is a compound of formula (I) or formula (II) or any variation thereof, or a pharmaceutically acceptable salt, solvate, hydrate, or co-crystal thereof, or a mixture of any of the foregoing, or a pharmaceutical composition comprising such compound, for use in the treatment of lung cancer, glioma, head and/or neck cancer, salivary gland cancer, breast cancer, esophageal cancer, liver cancer, stomach (gastric) cancer, uterine cancer, cervical cancer, biliary tract cancer, pancreatic cancer, colorectal cancer, renal cancer, bladder cancer, or prostate cancer. In some embodiments, the lung cancer is non-small cell lung cancer.

In another embodiment, provided herein is a compound of formula (I) or formula (II) or any variation thereof, or a pharmaceutically acceptable salt, solvate, hydrate, or co-crystal thereof, or a mixture of any of the foregoing, for use in the manufacture of a medicament for the treatment of cancer. In another embodiment, provided herein is a compound of formula (I) or formula (II) or any variation thereof, or a pharmaceutically acceptable salt, solvate, hydrate, or co-crystal thereof, or a mixture of any of the foregoing, for use in the manufacture of a medicament for the treatment of cancer, wherein the cancer comprises cells or cell tissue having increased ErbB2 kinase activity. In another embodiment, provided herein is a compound of formula (I) or formula (II) or any variation thereof, or a pharmaceutically acceptable salt, solvate, hydrate, or co-crystal thereof, or a mixture of any of the foregoing, for use in the manufacture of a medicament for the treatment of cancer, wherein the cancer cells or cancer cell tissue comprise one or more mutations in Exon 20 of the ErbB2. In some embodiments, the medicament is for the treatment of cancer, wherein the cancer cells comprise one or more genetic alterations in Exon 20 of the ErbB2 that introduce certain amino acid deletions and/or insertions selected from the group consisting of A775_A776insYVMA, G778_P780insGSP, G776delinsVC, P780_Y781insGSP, M774delinsWLV, A775_G776insSVMA, A775_G776insI, G776delinsLC, G778_S779InsCPG, and V777_G778insGSP. In another embodiment, provided herein is a compound of formula (I) or formula (II) or any variation thereof, or a pharmaceutically acceptable salt, solvate, hydrate, or co-crystal thereof, or a mixture of any of the foregoing, for use in the manufacture of a medicament for the treatment of cancer, wherein the cancer cells or cancer cell tissue comprise one or more disease-associated point mutations in ErbB2. In some embodiments, the medicament is for the treatment of cancer, wherein the cancer cells comprise one or more point mutations that introduce certain amino acid substitutions selected from the group consisting of: P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, N1219S, and A1232fs. In some embodiments, the medicament is for the treatment of cancer, wherein the cancer cells comprise one or more point mutations that introduce (a) an amino acid substitution selected from the group consisting of: P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, and N1219S; or (b) a frameshift at A1232. In some embodiments, the medicament is for the treatment of lung cancer, glioma, head and/or neck cancer, salivary gland cancer, breast cancer, esophageal cancer, liver cancer, stomach (gastric) cancer, uterine cancer, cervical cancer, biliary tract cancer, pancreatic cancer, colorectal cancer, renal cancer, bladder cancer, or prostate cancer. In some embodiments, the medicament is for the treatment of non-small cell lung cancer.

In some embodiments, the individual is a mammal. In some embodiments, the individual is a primate, dog, cat, rabbit, or rodent. In some embodiments, the individual is a primate. In some embodiments, the individual is a human. In some embodiments, the human is at least about or is about any of 18, 21, 30, 50, 60, 65, 70, 75, 80, or 85 years old. In some embodiments, the human is a child. In some embodiments, the human is less than about or about any of 21, 18, 15, 10, 5, 4, 3, 2, or 1 years old.

In some embodiments, the method further comprises administering one or more additional pharmaceutical agents. In some embodiments, the method further comprises administering one or more additional anti-cancer agents to the patient. In some embodiments, the method further comprises administering radiation. In some embodiments, the method further comprises administering one or more additional pharmaceutical agents and radiation.

### V. Dosing and Method of Administration

The dose of a compound described herein, or a pharmaceutically acceptable salt, solvate, hydrate, or co-crystal thereof, or a mixture of any of the foregoing, administered to an individual (such as a human) may vary with the particular compound or salt thereof, the method of administration, and the particular cancer, such as type and stage of cancer, being treated. In some embodiments, the amount of the compound, or a pharmaceutically acceptable salt, solvate, hydrate, or co-crystal thereof, or a mixture of any of the foregoing, is a therapeutically effective amount.

The compounds provided herein, or a pharmaceutically acceptable salt, solvate, hydrate, or co-crystal thereof, or a mixture of any of the foregoing, may be administered to an individual via various routes, including, e.g., intravenous, intramuscular, subcutaneous, oral, and transdermal.

The effective amount of the compound may in one aspect be a dose of between about 0.01 and about 100 mg/kg. Effective amounts or doses of the compounds of the present disclosure may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

Any of the methods provided herein may in one aspect comprise administering to an individual a pharmaceutical composition that contains an effective amount of a compound provided herein, or a pharmaceutically acceptable salt, solvate, hydrate, or co-crystal thereof, or a mixture of any of the foregoing, and a pharmaceutically acceptable excipient.

A compound or composition provided herein may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

### VI. Articles of Manufacture and Kits

The present disclosure further provides articles of manufacture comprising a compound described herein, or a pharmaceutically acceptable salt, solvate, hydrate, or co-crystal thereof, or a mixture of any of the foregoing, a composition described herein, or one or more unit dosages described herein in suitable packaging. In certain embodiments, the article of manufacture is for use in any of the methods described herein. Suitable packaging is known in the art and includes, for example, vials, vessels, ampules, bottles, jars, flexible packaging and the like. An article of manufacture may further be sterilized and/or sealed.

The present disclosure further provides kits for carrying out the methods of the present disclosure, which comprises one or more compounds described herein or a composition comprising a compound described herein. The kits may employ any of the compounds disclosed herein. In one variation, the kit employs a compound described herein, or a pharmaceutically acceptable salt, solvate, hydrate, or co-crystal thereof, or a mixture of any of the foregoing, thereof. The kits may be used for any one or more of the uses described herein, and, accordingly, may contain instructions for the treatment of any disease or described herein, for example for the treatment of cancer, including lung, glioma, skin, head and neck, salivary gland, breast, esophageal, liver, stomach (gastric), uterine, cervical, biliary tract, pancreatic, colorectal, renal, bladder or prostate cancer. In some embodiments, the kit may contain instructions for the treatment of non-small cell lung cancer.

In certain embodiments of the foregoing, the cancer comprises cells or cell tissue having one or more mutations in Exon 20 of the ErbB2. In still further embodiments, the cancer cells or cancer cell tissue comprise one or more mutations in Exon 20 of the ErbB2 that introduce certain amino acid deletions and/or insertions selected from the group consisting of A775_A776insYVMA, G778_P780insGSP, G776delinsVC, P780_Y781insGSP, M774delinsWLV, A775_G776insSVMA, A775_G776insI, G776delinsLC, G778_S779InsCPG, and V777_G778insGSP. In certain other embodiments of the cancer comprises cells or cell tissue having one or more disease-associated point mutations in ErbB2. In still further embodiments, the cancer cells or cancer cell tissue comprise the one or more point mutations that introduce amino acid substitutions selected from the group consisting of P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, N1219S, and A1232fs. In still further embodiments, the cancer cells or cancer cell tissue comprise the one or more point mutations that introduce (a) an amino acid substitution selected from the group consisting of P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, and N1219S; or (b) a frameshift at A1232.

The kits optionally further comprise a container comprising one or more additional pharmaceutical agents and which kits further comprise instructions on or in the package insert for treating the subject with an effective amount of the one or more additional pharmaceutical agents.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

### VII. General Synthetic Methods

The compounds of the present disclosure may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

The intermediates described in the following preparations may contain a number of nitrogen, hydroxy, and acid protecting groups such as esters. The variable protecting group may be the same or different in each occurrence depending on the particular reaction conditions and the particular transformations to be performed. The protection and deprotection conditions are well known to the skilled artisan and are described in the literature. See. e.g., Greene and Wuts, Protective Groups in Organic Synthesis, (T. Greene and P. Wuts, eds., 2d ed. 1991).

Certain stereochemical centers have been left unspecified and certain substituents have been eliminated in the following schemes for the sake of clarity and are not intended to limit the teaching of the schemes in any way. Furthermore, individual isomers, enantiomers, and diastereomers may be separated or resolved by one of ordinary skill in the art at any convenient point in the synthesis of compounds of the invention, by methods such as selective crystallization techniques or chiral chromatography (See for example, J. Jacques, et al., “Enantiomers, Racemates, and Resolutions”, John Wiley and Sons, Inc., 1981, and E. L. Eliel and S. H. Wilen, “Stereochemistry of Organic Compounds”, Wiley-Interscience, 1994).

The compounds of the present invention, or salts thereof, may be prepared by a variety of procedures known in the art, some of which are illustrated in the Examples below. The specific synthetic steps for each of the routes described may be combined in different ways, to prepare compounds of the invention, or salts thereof. The products of each step can be recovered by conventional methods well known in the art, including extraction, evaporation, precipitation, chromatography, filtration, trituration, and crystallization. The reagents and starting materials are readily available to one of ordinary skill in the art. Others may be made by standard techniques of organic and heterocyclic chemistry which are analogous to the syntheses of known structurally-similar compounds and the procedures described in the Examples which follow including any novel procedures.

Compounds of formulae (I), (I-a), (I-b), (I-c), (I-d), (I-e), (I-a-1), (I-a-2), (I-a-3), (I-a-4), (I-a-5), (I-a-6), (I-a-7), (I-b-1), (I-b-2), (I-b-3), (I-b-4), (I-b-5), (I-b-6), (I-b-7), (I-c-1), (I-c-2), (I-c-3), (I-c-4), (I-c-5), (I-c-6), (I-c-7), (I-d-1), (I-d-2), (I-d-3), (I-d-4), (I-d-5), (I-d-6), (I-d-7), (I-e-1), (I-e-2), (I-e-3), (I-e-4), (I-e-5), (I-e-6), (I-e-7), (II), (II-a), (II-b), (II-c), (II-a-1), (II-a-2), (II-a-3), (II-b-1), (II-b-2), (II-b-3), (II-c-1), (II-c-2), (II-c-3), (II-d-1), (II-d-2), (II-d-3), (II-d-4), (II-d-5), (II-d-6), (II-d-7), (II-d-8), (II-d-9), (II-d-10), (II-d-11), (II-d-12), (II-d-13), (II-d-14), (II-d-15), (II-d-16), (II-d-17), (II-d-18), (II-e-1), (II-e-2), (II-e-3), (II-e-4), (II-e-5), (II-e-6), (II-e-7), (II-e-8), (II-e-9), (II-e-10), (II-e-11), (II-e-12), (II-e-13), (II-e-14), (II-e-15), (II-e-16), (II-e-17), (II-e-18), (II-f-1), (II-f-2), (II-f-3), (II-f-4), (II-f-5), (II-f-6), (II-f-7), (II-f-8), (II-f-9), (II-f-10), (II-f-11), (II-f-12), (II-f-13), (II-f-14), (II-f-15), (II-f-16), (II-f-17), (II-f-18), (II-g-1), (II-g-2), (II-g-3), (II-g-4), (II-g-5), or (II-g-6) can be prepared according to Scheme A, Scheme B, Scheme C, Scheme D, Scheme E, Scheme F, Scheme G, Scheme H, Scheme I, Scheme J, Scheme K, Scheme L, Scheme M, or Scheme N, wherein the Ring A moiety, R¹, R², R³, R⁴, R⁵, R⁶, R⁷, R⁸, G, X, Y, and Z are as defined for formula (I) or formula (II) or any applicable variation thereof as detailed herein.

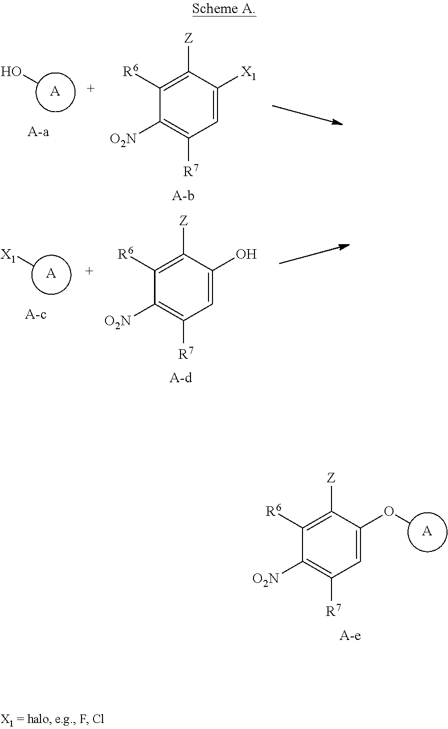

As shown in Scheme A, nucleophilic substitution by hydroxylated Ring A-containing heterocycles of general formula A-a of nitrosylated benzenes of general formula A-b provides the coupled ether compounds of general formula A-e. Alternatively, compounds of general formula A-c may be reacted with compounds of general formula A-d to yield the coupled ether compounds of general formula A-e.

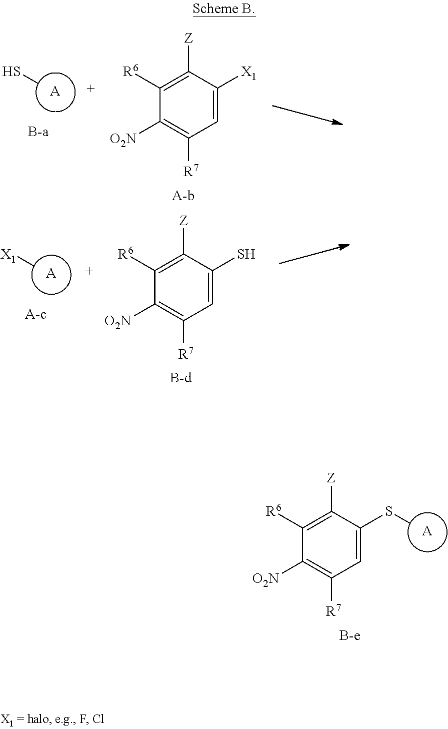

As shown in Scheme B, nucleophilic substitution by thiolated Ring A-containing heterocycles of general formula B-a of nitrosylated benzenes of general formula A-b provides the coupled thioether compounds of general formula B-e. Alternatively, compounds of general formula A-c may be reacted with compounds of general formula B-d to yield the coupled thioether compounds of general formula B-e.

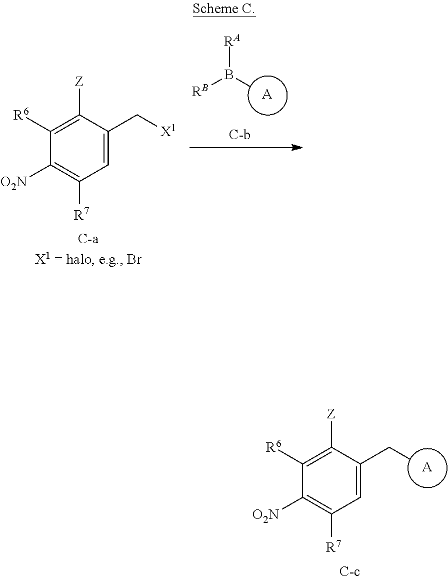

As shown in Scheme C, compounds of general formula C-a can be coupled with a suitable Ring A-substituted boronic acid derivative C-b, wherein R^{A} and R^{B} are independently selected from the group consisting of OH and O—(C₁-C₆ alkyl), or R^{A} and R^{B} are taken together with the boron atom to which they are attached to form a 5-10 membered heterocycle, to afford compounds of general formula C-c. Compounds of C-a may also be substituted with suitably substituted analogs (i.e., wherein the methylene carbon is substituted by —CH₃ or spirocyclopropyl) to provide analogous precursors of formula C-c for compounds of formula (I) wherein G is —CH(CH₃) or spirocyclopropyl.

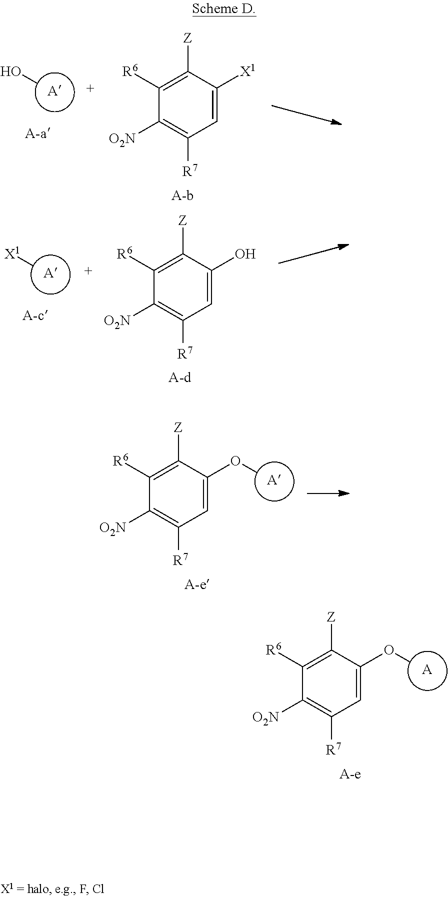

In one variation of Scheme A, as shown in Scheme D, the final Ring A may be prepared or formed from Ring A′, a precursor to Ring A, as part of the synthetic scheme. With reference to Scheme C, nucleophilic substitution by hydroxylated Ring A′-containing heterocycles of general formula A-a′ of nitrosylated benzenes of general formula A-b provides the coupled ether compounds of general formula A-e′. Alternatively, compounds of general formula A-c′ may be reacted with compounds of general formula A-d to yield the coupled ether compounds of general formula A-e′. Ring A′ may be converted to Ring A, for example, by the conversion of formula A-e′ to formula A-e.

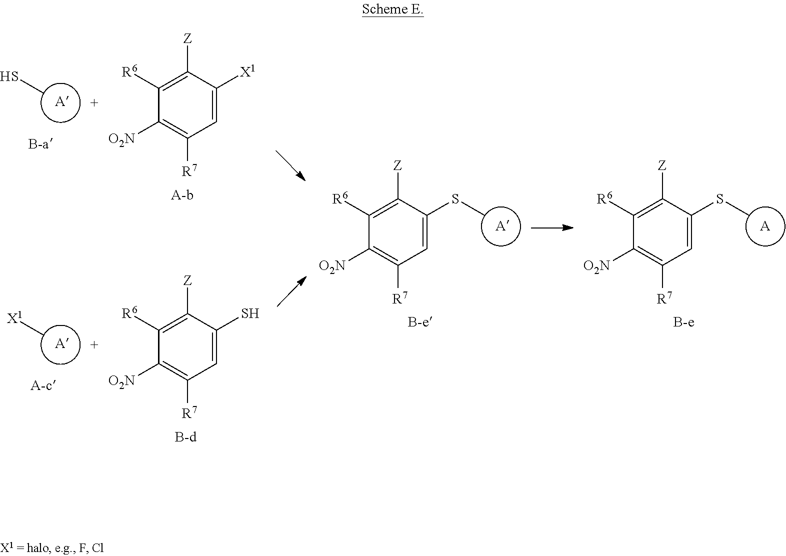

In one variation of Scheme B, as shown in Scheme E, the final Ring A may be prepared or formed from Ring A′, a precursor to Ring A, as part of the synthetic scheme. With reference to Scheme E, nucleophilic substitution by thiolated Ring A′-containing heterocycles of general formula B-a′ of nitrosylated benzenes of general formula A-b provides the coupled thioether compounds of general formula B-e′. Alternatively, compounds of general formula A-c′ may be reacted with compounds of general formula B-d to yield the coupled thioether compounds of general formula B-e′. Ring A′ may be converted to Ring A, for example, by the conversion of formula B-e′ to formula B-e.

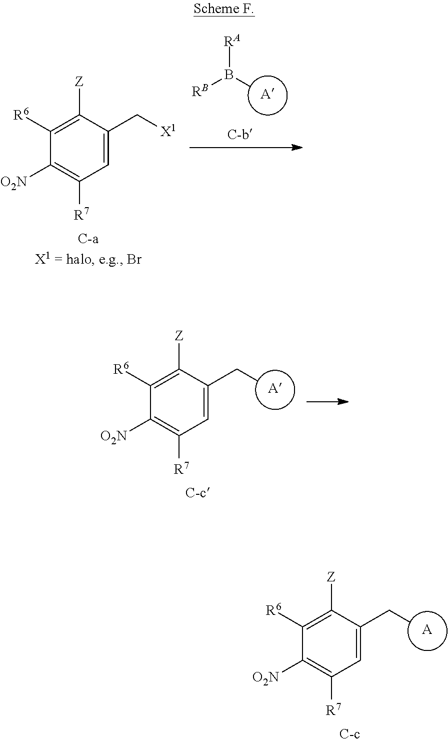

In one variation of Scheme C, as shown in Scheme F, the final Ring A may be prepared or formed from Ring A′, a precursor to Ring A, as part of the synthetic scheme. With reference to Scheme F, compounds of general formula C-a can be coupled with a suitable Ring A′-substituted boronic acid derivative C-b′, wherein R^{A} and R^{B} are independently selected from the group consisting of OH and O—(C₁-C₆ alkyl), or R^{A} and R^{B} are taken together with the boron atom to which they are attached to form a 5-10 membered heterocycle, to afford compounds of general formula C-c′. Ring A′ may be converted to Ring A, for example, by the conversion of formula C-c′ to formula C-c.

Ring A′ may encompass, but is not limited to, monocyclic heteroaryl rings such as pyridyl, pyrimidine, pyrazine. Exemplary reactions to convert Ring A′ to Ring A (in Scheme D, Scheme E, or Scheme F) may include, for example, reacting a monocyclic Ring A′ with a suitable substrate and cyclizing the substrate to form a bicyclic Ring A as shown in Scheme G, Scheme H, or Scheme I below, or adding one or more substituents to Ring A′ to give Ring A.

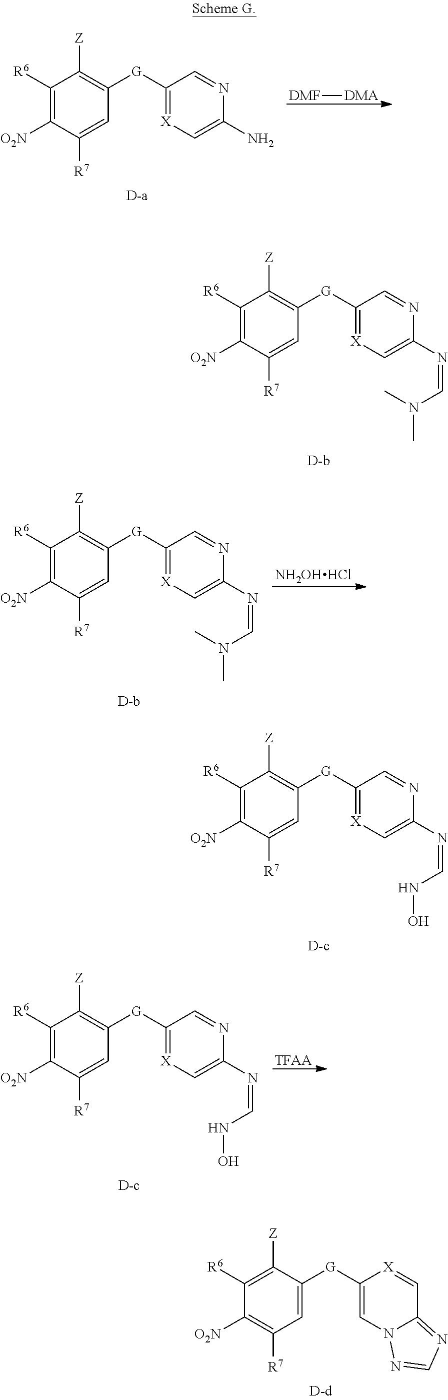

As shown in Scheme G, compounds of general formula D-a (corresponding to compounds of general formulae A-e′, B-e′, or C-c′) are reacted with N,N-dimethylformamide-dimethyl acetal (DMF-DMA) to give compounds of general formula D-b, and further reacting compounds of general formula D-b with ammonium hydroxide to yield compounds of general formula D-c. Compounds of general formula D-c are further cyclized, for example, in the presence of trifluoroacetic anhydride, to provide compounds of general formula D-d (corresponding to compounds of general formulae A-e, B-e, or C-c) having bicyclic Ring A.

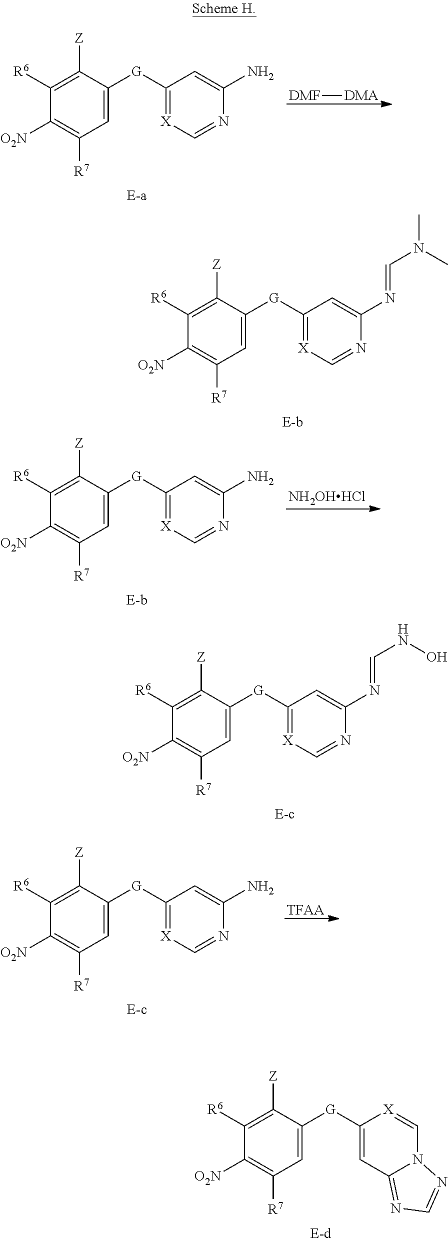

As shown in Scheme H, compounds of general formula E-a (corresponding to compounds of general formulae A-e′, B-e′, or C-c′) are reacted with N,N-dimethylformamide-dimethyl acetal to give compounds of general formula E-b, and further reacting compounds of general formula E-b with ammonium hydroxide to yield compounds of general formula E-c. Compounds of general formula E-c are further cyclized, for example, in the presence of trifluoroacetic anhydride, to provide compounds of general formula E-d (corresponding to compounds of general formulae A-e, B-e, or C-c) having bicyclic Ring A.

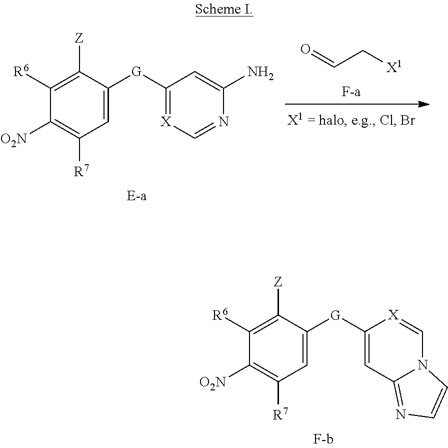

As shown in Scheme I, compounds of general formula E-a (corresponding to compounds of general formulae A-e′, B-e′, or C-c′) are reacted with compounds of general formula F-a to give compounds of general formula F-b (corresponding to compounds of general formulae A-e, B-e, or C-c) having bicyclic Ring A.

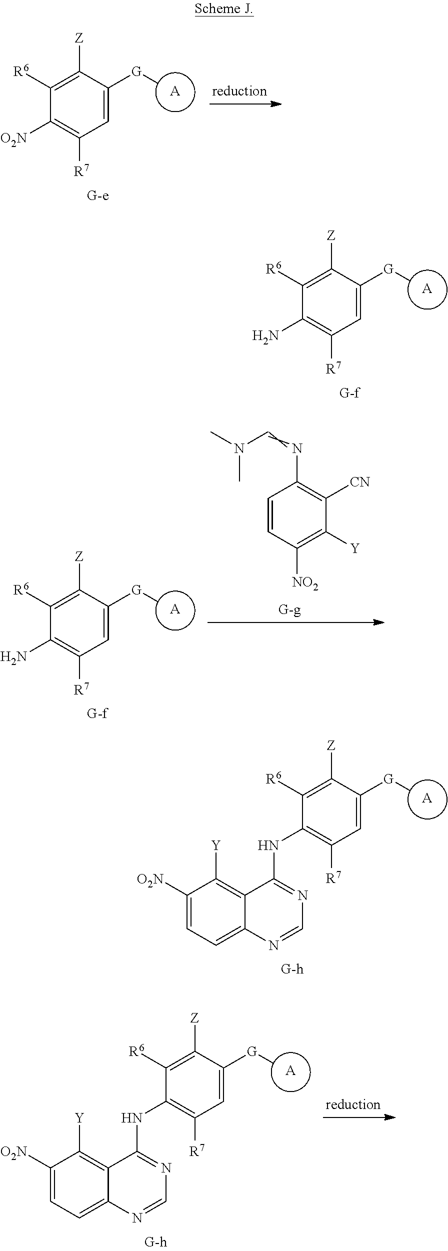

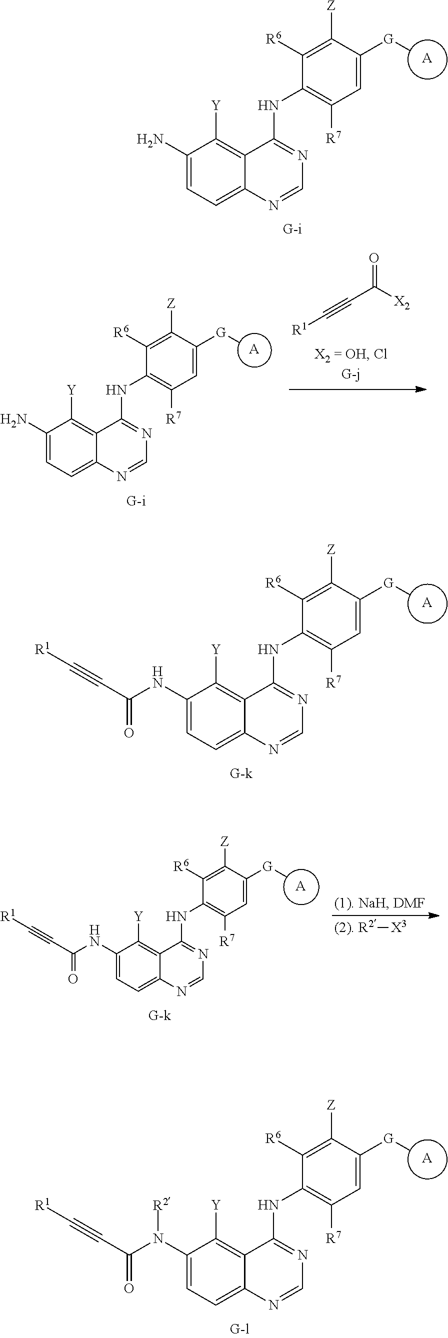

Compounds of formula G-1 may be prepared according to the general synthetic scheme shown in Scheme J. The nitrosyl group on the compounds of general formula G-e (corresponding to compounds of general formulae A-e, B-e, or C-c) is reduced (for example, in the presence of H₂ and Pd/C) to give compounds of general formula G-f. The compounds of general formula G-f are reacted with compounds of general formula G-g to yield intermediate compounds of general formula G-h. Compounds of general formula G-h are reduced (for example, in the presence of H₂ and Pd/C) to give compounds of general formula G-i. The compounds of general formula G-i are reacted with alkynyl carboxylic acids or alkynyl acid halides of formula G-j to yield compounds of formula G-k (corresponding to compounds of formula (II) wherein R² is H, or formula (I) wherein R⁸ is H and R² is H). Compounds of general formula G-k can be deprotonated with sodium hydride to allow further substitution of the amide nitrogen (e.g., alkylation with an R²′ substrate having a suitable leaving group X³, such as methyl iodide) to afford compounds of general formula G-1 (corresponding to compounds of formula (I) or formula (II), wherein R²′ is equivalent to R² except that R²′ does not include hydrogen).

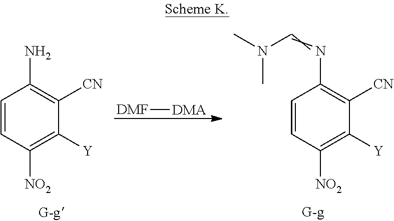

The compounds of formula G-g may be prepared from the reaction of a suitable precursor G-g′ with N,N-dimethylformamide dimethyl acetal (DMF-DMA), as shown in Scheme K above.

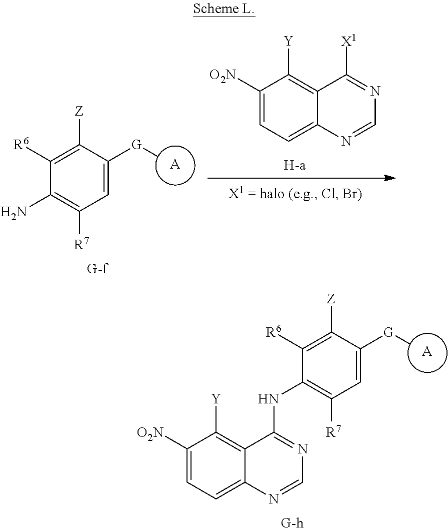

An alternative to the synthesis of compounds of general formula G-h is provided in Scheme L above. The compounds of general formula G-f are reacted with compounds of general formula H-a to give compounds of general formula G-h.

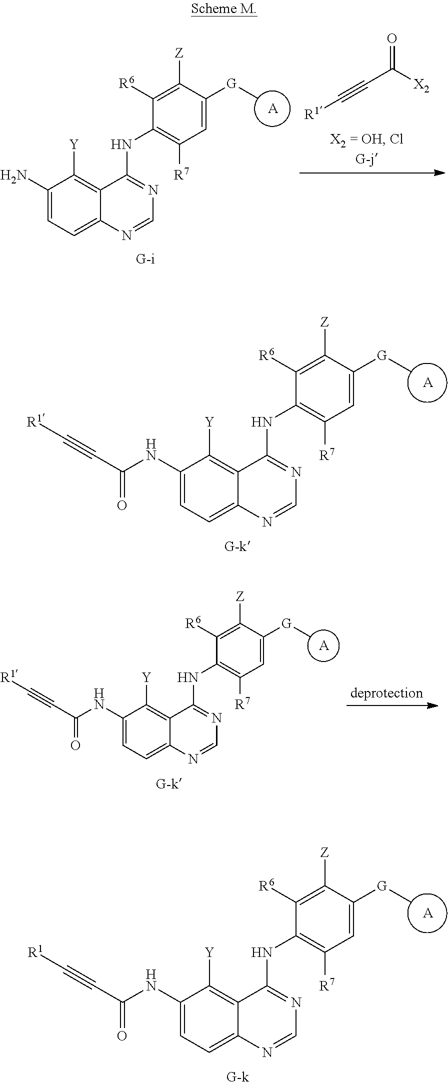

As shown in Scheme M, compounds of general formula G-i are reacted with alkynyl carboxylic acids or alkynyl acid halides of formula G-j′ (wherein R¹′ is a precursor to R¹) to yield compounds of formula G-k′. The compounds of formula G-j′ may be obtained from commercially available starting materials. For example, the protected R¹′ alkynes may be reacted with protected acid halides or carboxylic acid to give alkynyl carboxylic acids or alkynyl acid halides. Compounds of general formula G-k′ can be converted (for example, via deprotection) to afford compounds of general formula G-k (corresponding to compounds of formula (I), wherein R² is H and R⁸ is H, or formula (II) wherein R² is H).

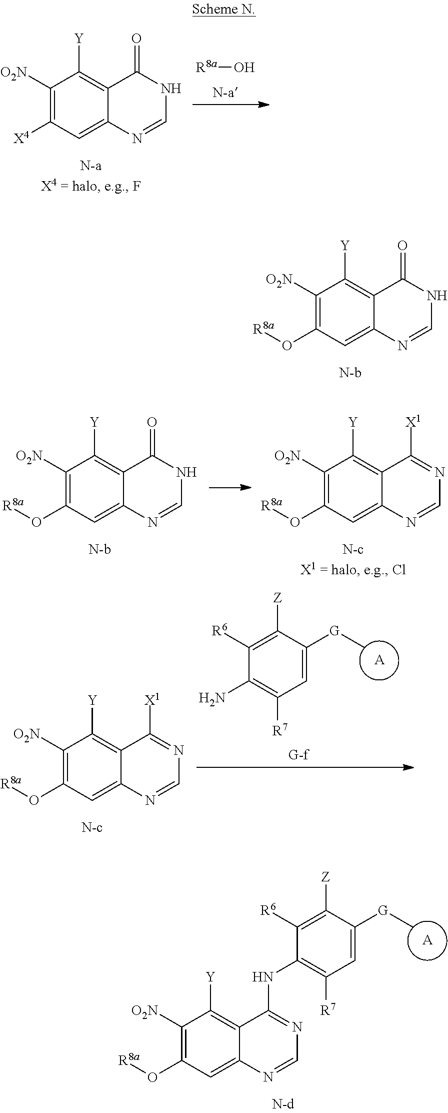

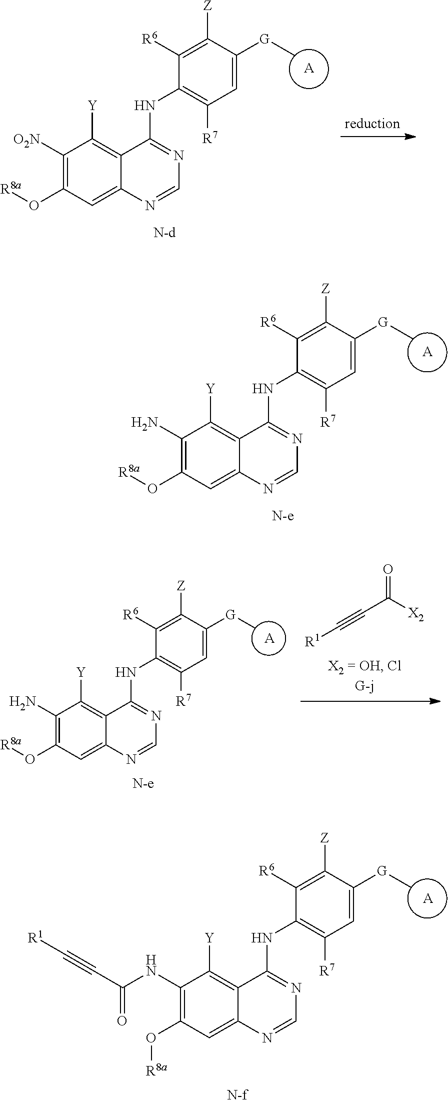

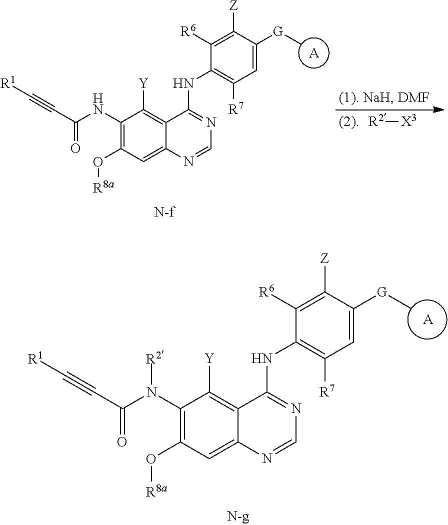

Compounds of formula N-g, wherein R^{8a} is C₁-C₃ alkyl optionally substituted by 1-4 substituents selected from the group consisting of —F, —OH, —R^{8a}, and —NR^{8a}R^{8b}, wherein each R^{8a} and R^{8b} are independently C₁-C₃ alkyl or wherein each pair of geminal R^{8a} and R^{8b} may be taken together with the nitrogen atom to which they are attached to form an N-heterocycloalkyl, may be prepared according to the general synthetic scheme shown in Scheme N. The compounds of general formula N-a are reacted with compounds of general formula N-a′ to yield intermediate compounds of general formula N-b. Compounds of general formula N-b are halogenated (for example, in the presence of SOCl₂ and POCl₃) to give compounds of general formula N-c. The compounds of general formula N-c are reacted with compounds of general formula G-f to yield intermediate compounds of general formula N-d. Compounds of general formula N-d are reduced (for example, in the presence of H₂ and Pd/C) to give compounds of general formula N-e. The compounds of general formula N-e are reacted with alkynyl carboxylic acids or alkynyl acid halides of formula G-j to yield compounds of formula N-f (corresponding to compounds of formula (I), wherein R² is H and R⁸ is —OR^{8a}). Compounds of general formula N-f can be deprotonated with sodium hydride to allow further substitution of the amide nitrogen (e.g., alkylation with an R²′ substrate having a suitable leaving group X³, such as methyl iodide) to afford compounds of general formula N-g (corresponding to compounds of formula (I), wherein R²′ is equivalent to R² except that R²′ does not include hydrogen and R⁸ is —OR^{8a}).

### EXAMPLES

It is understood that the present disclosure has been made only by way of example, and that numerous changes in the combination and arrangement of parts can be resorted to by those skilled in the art without departing from the spirit and scope of present disclosure.

The chemical reactions in the Examples described can be readily adapted to prepare a number of other compounds disclosed herein, and alternative methods for preparing the compounds of this disclosure are deemed to be within the scope of this disclosure. For example, the synthesis of non-exemplified compounds according to the present disclosure can be successfully performed by modifications apparent to those skilled in the art, e.g., by appropriately protecting interfering groups, by utilizing other suitable reagents known in the art other than those described, or by making routine modifications of reaction conditions, reagents, and starting materials. Alternatively, other reactions disclosed herein or known in the art will be recognized as having applicability for preparing other compounds of the present disclosure.

Abbreviations used in the Examples include the following: AcOH: acetic acid; BSA: bovine serum albumin; DCM: dichloromethane; DIEA: diisopropylethylamine; DMF: dimethylformamide; DMF-DMA: dimethylformamide-dimethyl acetal; DMSO: dimethyl sulfoxide; DTT: dithiothreitol; ESI: electrospray ionization; EGTA: ethylene glycol-bis(O-aminoethyl ether)-N,N,N′,N′-tetraacetic acid; EtOAc: ethyl acetate; EtOH: ethanol or ethyl alcohol; ¹H NMR: proton nuclear magnetic resonance; HATU: 1-[Bis(dimethylamino)methylene]-1H-1,2,3-triazolo[4,5-b]pyridinium 3-oxide hexafluorophosphate (Hexafluorophosphate Azabenzotriazole Tetramethyl Uronium); LCMS: liquid chromatography—mass spectrometry; LiHMDS: lithium hexamethyldisilazide; MeOH: methanol or methyl alcohol; n-BuLi: n-butyllithium; NMP: N-methylpyrrolidone; PBS: phosphate-buffered saline; PBST: PBS with Tween 20; Py: pyridine; TFAA: trifluoroacetic anhydride; THF: tetrahydrofuran; and TLC: thin-layer chromatography.

### Example S1. Synthesis of N-[4-[(3-methyl-4-[[1,2,4]triazolo[1,5-a]pyridin-7-yloxy]phenyl)amino]quinazolin-6-yl]but-2-ynamide (Compound 1)

### Step 1: Synthesis of 4-(2-methyl-4-nitrophenoxy)pyridin-2-amine

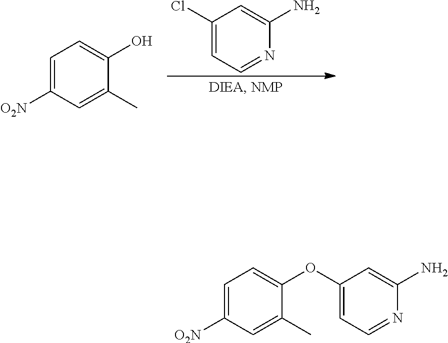 [SMILES: Cc1cc([N+](=O)[O-])ccc1O ; Nc1cc(Cl)ccn1 ; Cc1cc([N+](=O)[O-])ccc1Oc1ccnc(N)c1]

To a solution of 4-nitro-o-cresol (10.0 g, 65.30 mmol) in NMP (50.0 mL) was added 4-chloropyridin-2-amine (8.4 g, 65.30 mmol) and DIEA (16.9 g, 130.60 mmol) at room temperature. The resulting mixture was stirred at 150° C. for 24 h. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/8, v/v) to afford 4-(2-methyl-4-nitrophenoxy)pyridin-2-amine (2.9 g, 18%) as a brown solid. LCMS (ESI, m/z): [M+H]⁺=246.1.

### Step 2: Synthesis of (E)-N-hydroxy-N′-(4-(2-methyl-4-nitrophenoxy)pyridin-2-yl)formimidamide

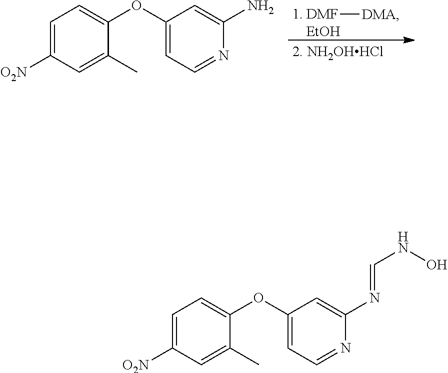

To a solution of 4-(2-methyl-4-nitrophenoxy)pyridin-2-amine (2.9 g, 11.83 mmol) in EtOH (30.0 mL) was added DMF-DMA (2.4 g, 20.10 mmol) at room temperature. The resulting mixture was stirred at 75° C. for 3 h. Then hydroxylamine hydrochloride (2.9 g, 41.39 mmol) was added to the mixture at 40° C. The resulting mixture was stirred at 50° C. for 0.5 h. After the reaction was completed, the reaction mixture was cooled to room temperature and then filtered. The solid was washed with EtOH and dried to afford (E)-N-hydroxy-N′-(4-(2-methyl-4-nitrophenoxy)pyridin-2-yl)formimidamide (2.7 g, crude) as a light brown solid. LCMS (ESI, m/z): [M+H]⁺=289.1.

### Step 3: Synthesis of 7-(2-methyl-4-nitrophenoxy)-[1,2,4]triazolo[1,5-a]pyridine

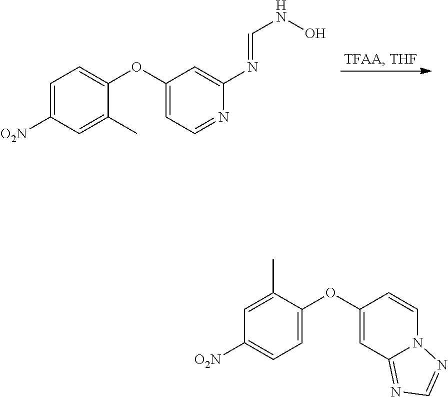 [SMILES: Cc1cc([N+](=O)[O-])ccc1Oc1ccnc(/N=C/NO)c1 ; Cc1cc([N+](=O)[O-])ccc1Oc1ccn2ncnc2c1]

To a solution of (E)-N-hydroxy-N′-(4-(2-methyl-4-nitrophenoxy)pyridin-2-yl)formimidamide (2.8 g, crude) in THF (40.0 mL) was added TFAA (8.0 g, 38.16 mmol) at 0° C. The resulting mixture was stirred at room temperature for 5 h. After the reaction was completed, the reaction was diluted with EtOAc and washed with NaHCO₃(aq), dried over anhydrous sodium sulfate and filtered. The filtrate was concentrated under reduced pressure. The residue was purified by flash column chromatography with petroleum ether/ethyl acetate (2/4, v/v) to afford 7-(2-methyl-4-nitrophenoxy)-[1,2,4]triazolo[1,5-a]pyridine (460.0 mg, 17%) as a white solid. LCMS (ESI, m/z): [M+H]⁺=271.1.

### Step 4: Synthesis of 3-methyl-4-[[1,2,4]triazolo[1,5-a]pyridin-7-yloxy]aniline

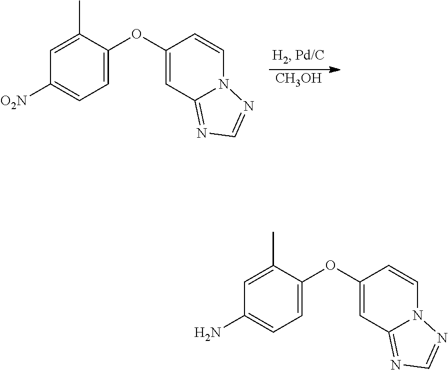

To a solution of 7-(2-methyl-4-nitrophenoxy)-[1,2,4]triazolo[1,5-a]pyridine (410.0 mg, 1.52 mmol) in MeOH (10.0 mL) was added Pd/C (120.0 mg, dry). The mixture was stirred at at room temperature for 3 h under H₂. After the reaction was completed, the resulting mixture was filtered. The filtrate was concentrated under reduced pressure to afford 3-methyl-4-[[1,2,4]triazolo[1,5-a]pyridin-7-yloxy]aniline (360.0 mg, crude) as a yellow solid. LCMS (ESI, m/z): [M+H]⁺=241.1.

### Step 5: Synthesis of N′-(2-cyano-4-nitrophenyl)-N,N-dimethylformimidamide

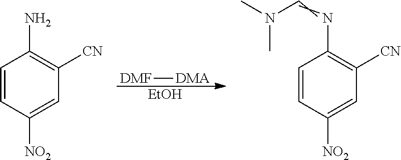

To a solution of 2-amino-5-nitrobenzonitrile (5.0 g, 30.65 mmol) in EtOH (20.0 mL) was added DMF-DMA (5.8 g, 52.11 mmol) at room temperature. The resulting mixture was stirred at 75° C. for 3 h. After the reaction was completed, the reaction mixture was cooled to room temperature and then filtered. The solid was collected and dried to afford N′-(2-cyano-4-nitrophenyl)-N,N-dimethylformimidamide (5.8 g, crude) as a yellow solid. LCMS (ESI, m/z): [M+H]⁺=219.1.

### Step 6: Synthesis of N-(3-methyl-4-[[1,2,4]triazolo[1,5-a]pyridin-7-yloxy]phenyl)-6-nitroquinazolin-4-amine

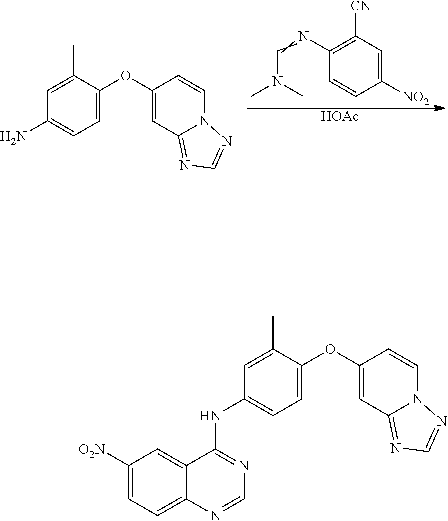 [SMILES: Cc1cc(N)ccc1Oc1ccn2ncnc2c1 ; CN(C)C=Nc1ccc([N+](=O)[O-])cc1C#N ; Cc1cc(Nc2ncnc3ccc([N+](=O)[O-])cc23)ccc1Oc1ccn2ncnc2c1 ; "HOAc"]

To a solution of 3-methyl-4-[[1,2,4]triazolo[1,5-a]pyridin-7-yloxy]aniline (310.0 mg, 1.29 mmol) in CH₃COOH (5.0 mL) was added N′-(2-cyano-4-nitrophenyl)-N,N-dimethylformimidamide (281.6 mg, 1.29 mmol) at room temperature. The resulting mixture was stirred at 85° C. for 2 h. After the reaction was completed, the resulting mixture was concentrated under reduced pressure. The resulting mixture was diluted with ice water and filtered. The solid was washed with H₂O and dried to afford N-(3-methyl-4-[[1,2,4]triazolo[1,5-a]pyridin-7-yloxy]phenyl)-6-nitroquinazolin-4-amine (530.0 mg, crude) as a yellow solid. LCMS (ESI, m/z): [M+H]⁺=414.1.

### Step 7: Synthesis of N4-(3-methyl-4-[[1,2,4]triazolo[1,5-a]pyridin-7-yloxy]phenyl)quinazoline-4,6-diamine

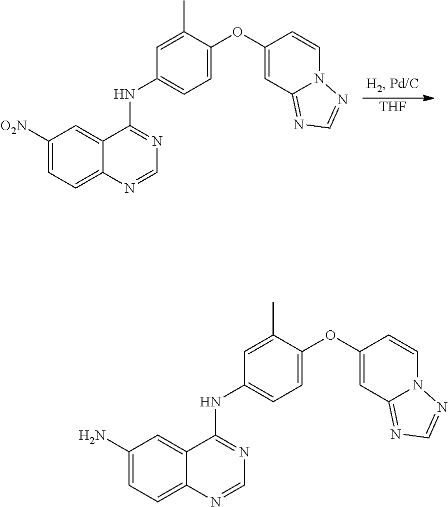 [SMILES: Cc1cc(Nc2ncnc3ccc([N+](=O)[O-])cc23)ccc1Oc1ccn2ncnc2c1 ; Cc1cc(Nc2ncnc3ccc(N)cc23)ccc1Oc1ccn2ncnc2c1 ; "THF"]

To a solution of N-(3-methyl-4-[[1,2,4]triazolo[1,5-a]pyridin-7-yloxy]phenyl)-6-nitroquinazolin-4-amine (535.0 mg, 1.29 mmol) in THF (20.0 mL) was added Pd/C (161.1 mg, dry). The resulting mixture was stirred at room temperature for 3 h under H₂. After the reaction was completed, the resulting mixture was filtered. The filtrate was concentrated under reduced pressure to afford N4-(3-methyl-4-[[1,2,4]triazolo[1,5-a]pyridin-7-yloxy]phenyl)quinazoline-4,6-diamine (360.0 mg, crude) as a yellow solid. LCMS (ESI, m/z): [M+H]⁺=384.1.

### Step 8a: Synthesis of N-[4-[(3-methyl-4-[[1,2,4]triazolo[1,5-a]pyridin-7-yloxy]phenyl)amino]quinazolin-6-yl]but-2-ynamide (Compound 1)

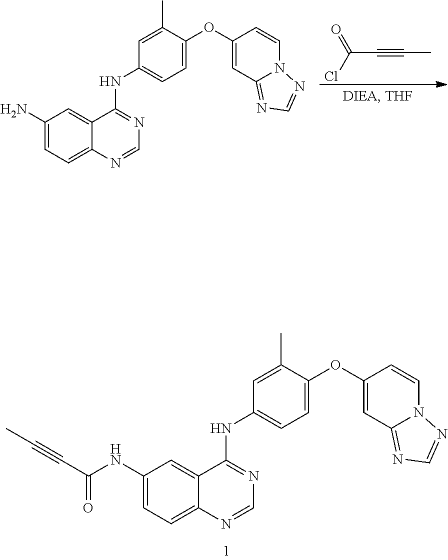

To a solution of N4-(3-methyl-4-[[1,2,4]triazolo[1,5-a]pyridin-7-yloxy]phenyl)quinazoline-4,6-diamine (260.0 mg, 0.68 mmol) in THF (10.0 mL) was added but-2-ynoyl chloride (76.5 mg, 0.75 mmol) and DIEA (105.2 mg, 0.81 mmol) at room temperature. The resulting mixture was stirred at room temperature for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 19×250 mm, 5 um; Mobile Phase A: Water (10 mmol/L NH₄HCO₃), Mobile Phase B: MeOH-HPLC; Flow rate: 25 mL/min; Gradient: 55% B to 85% B in 7 min; 254 nm) to afford N-[4-[(3-methyl-4-[[1,2,4]triazolo[1,5-a]pyridin-7-yloxy]phenyl)amino]quinazolin-6-yl]but-2-ynamide (Compound 1) (17.4 mg, 5%) as a yellow solid. LCMS (ESI, m/z): [M+H]⁺=450.2. ¹H NMR (300 MHz, DMSO-d₆): δ 10.95 (s, 1H), 9.89 (s, 1H), 8.96-8.93 (m, 1H), 8.77-8.66 (m, 1H), 8.55 (s, 1H), 8.38 (s, 1H), 7.94-7.79 (m, 4H), 7.21 (d, J=8.7 Hz, 1H), 7.12-7.03 (m, 1H), 6.81 (s, 1H), 2.40-2.10 (m, 6H).

### Step 8b: Alternative Synthesis of N-[4-[(3-methyl-4-[[1,2,4]triazolo[1,5-a]pyridin-7-yloxy]phenyl)amino]quinazolin-6-yl]but-2-ynamide (Compound 1)

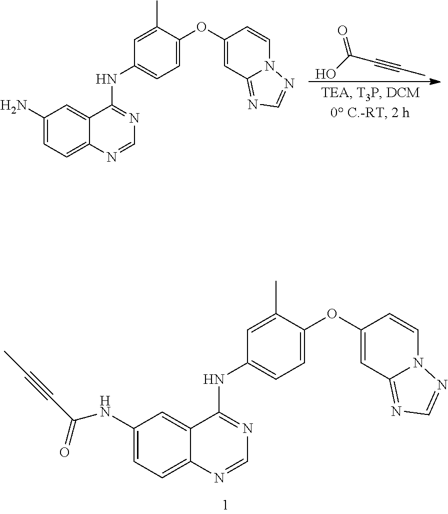

In an alternative to Step 8a: To a solution of N4-(3-methyl-4-[[1,2,4]triazolo[1,5-a]pyridin-7-yloxy]phenyl)quinazoline-4,6-diamine (12 g, 31.3 mmol), but-2-ynoic acid (5.26 g, 62.6 mmol) and TEA (15.8 g, 156.5 mmol) in DCM (300 mL) was added T₃P (59.7 g, 93.9 mmol, 50% ethyl acetate solution) at 0° C. and stirred at room temperature for 2 h. Then, the resulting mixture was quenched with sodium bicarbonate solution. The resulting solution was extracted with DCM and the organic layers were combined. The organic layer was dried over anhydrous sodium sulfate and concentrated in vacuum. The residue was purified by flash chromatography on silica gel eluting with DCM/MeOH (10/1) to afford crude product. Then, the crude product was washed with acetonitrile. After filtration, the solid was collected to afford N-[4-[(3-methyl-4-[[1,2,4]triazolo[1,5-a]pyridin-7-yloxy]phenyl)amino]quinazolin-6-yl]but-2-ynamide (Compound 1) (6.0242 g, 42.8% yield) as a light yellow solid. LCMS (ESI, m/z): [M+H]⁺=450.35; ¹H NMR (400 MHz, DMSO-d₆) δ 10.94 (s, 1H), 9.88 (s, 1H), 8.94 (dd, J=7.5, 0.7 Hz, 1H), 8.81-8.72 (m, 1H), 8.55 (s, 1H), 8.39 (s, 1H), 7.86-7.73 (m, 4H), 7.20 (d, J=8.7 Hz, 1H), 7.04 (dd, J=7.5, 2.6 Hz, 1H), 6.81 (d, J=2.5 Hz, 1H), 2.19 (s, 3H), 2.10 (s, 3H).

### Example S2. Synthesis of N-[4-[(4-[imidazo[1,2-a]pyridin-7-yloxy]-3-methylphenyl)amino]quinazolin-6-yl]but-2-ynamide (Compound 2)

### Step 1: Synthesis of 7-(2-methyl-4-nitrophenoxy)imidazo[1,2-a]pyridine

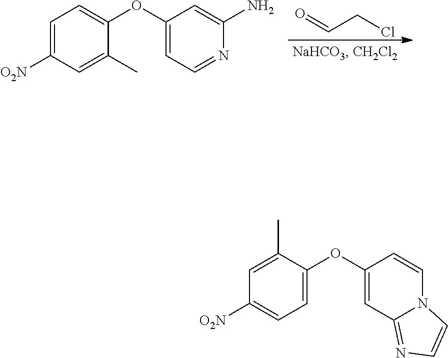

To a solution of 4-(2-methyl-4-nitrophenoxy)pyridin-2-amine (4.8 g, 19.57 mmol) in CH₂Cl₂ (40.0 mL) was added aq·NaHCO₃ (40.0 mL) and chloroacetaldehyde (3.8 g, 48.93 mmol) at room temperature. The resulting mixture was stirred at room temperature for 16 h. After the reaction was completed, the reaction was extracted with CH₂Cl₂. The combined organic layer was washed with brine, dried over anhydrous sodium sulfate and filtered. The filtrate was concentrated under reduced pressure. The residue was purified by reverse phase flash column chromatography with ACN/H₂O (20/80, v/v) to afford 7-(2-methyl-4-nitrophenoxy)imidazo[1,2-a]pyridine (1.2 g, 22%) as a yellow oil. LCMS (ESI, m/z): [M+H]⁺=270.1.

### Step 2: Synthesis of 4-[imidazo[1,2-a]pyridin-7-yloxy]-3-methylaniline

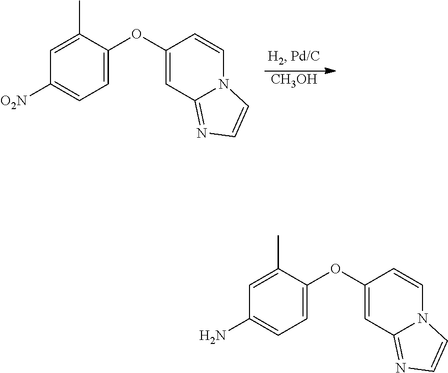

To a solution of 7-(2-methyl-4-nitrophenoxy)imidazo[1,2-a]pyridine (600.0 mg, 2.23 mmol) in MeOH (10.0 mL) was added Pd/C (213.4 mg, dry) at room temperature. The resulting mixture was stirred at room temperature for 2 h under H₂. After the reaction was completed, the resulting mixture was filtered. The filtrate was concentrated under reduced pressure to afford 4-[imidazo[1,2-a]pyridin-7-yloxy]-3-methylaniline (440.0 mg, crude) as a yellow solid. LCMS (ESI, m/z): [M+H]⁺=240.1.

### Step 3: Synthesis of N-(4-[imidazo[1,2-a]pyridin-7-yloxy]-3-methylphenyl)-6-nitroquinazolin-4-amine

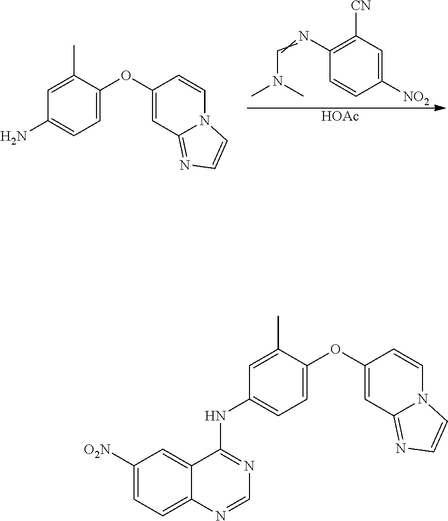 [SMILES: Cc1cc(N)ccc1Oc1ccn2ccnc2c1 ; CN(C)C=Nc1ccc([N+](=O)[O-])cc1C#N ; Cc1cc(Nc2ncnc3ccc([N+](=O)[O-])cc23)ccc1Oc1ccn2ccnc2c1 ; "HOAc"]

To a solution of 4-[imidazo[1,2-a]pyridin-7-yloxy]-3-methylaniline (1.7 g, 1.29 mmol) in HOAc (30.0 mL) was added N′-(2-cyano-4-nitrophenyl)-N,N-dimethylformimidamide (1.5 g, 6.89 mmol) at room temperature. The resulting mixture was stirred at 85° C. for 2 h. After the reaction was completed, the resulting mixture was concentrated under reduced pressure. The pH value of the mixture was adjusted to pH 7 with aq·NaHCO₃ and then filtered. The solid was collected and dried to afford N-(4-[imidazo[1,2-a]pyridin-7-yloxy]-3-methylphenyl)-6-nitroquinazolin-4-amine (2.5 g, crude) as a yellow solid. LCMS (ESI, m/z): [M+H]⁺=413.1.

### Step 4: Synthesis of N4-(4-[imidazo[1,2-a]pyridin-7-yloxy]-3-methylphenyl)quinazoline-4,6-diamine

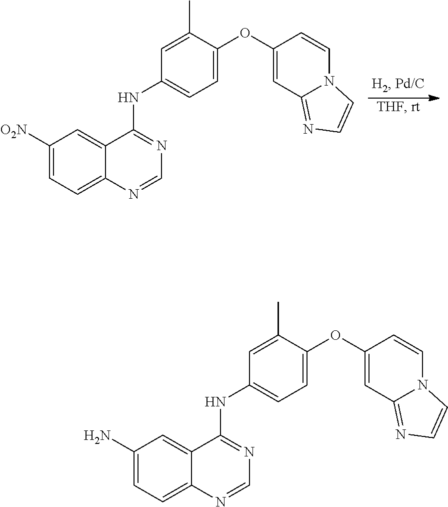

To a solution of N-(4-[imidazo[1,2-a]pyridin-7-yloxy]-3-methylphenyl)-6-nitroquinazolin-4-amine (450.0 mg, 1.10 mmol) in THF (20.0 mL) was added Pd/C (134.7 mg, dry) at room temperature. The resulting mixture was stirred at room temperature for 2 h under H₂. After the reaction was completed, the resulting mixture was filtered. The filtrate was concentrated under reduced pressure to afford N4-(4-[imidazo[1,2-a]pyridin-7-yloxy]-3-methylphenyl)quinazoline-4,6-diamine (380.0 mg, crude) as a yellow solid. LCMS (ESI, m/z): [M+H]⁺=383.2.

### Step 5: Synthesis of N-[4-[(4-[imidazo[1,2-a]pyridin-7-yloxy]-3-methylphenyl)amino]quinazolin-6-yl]but-2-ynamide (Compound 2)

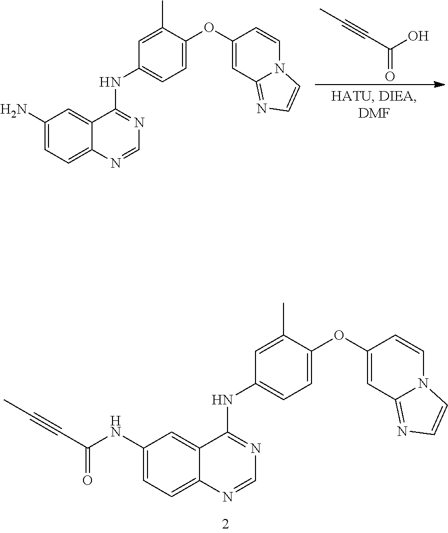

To a solution of N4-(4-[imidazo[1,2-a]pyridin-7-yloxy]-3-methylphenyl)quinazoline-4,6-diamine (200.0 mg, 0.52 mmol) in DMF (12.0 mL) was added 2-butynoic acid (52.8 mg, 0.63 mmol), DIEA (202.8 mg, 1.57 mmol) and HATU (278.4 mg, 0.73 mmol) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep Phenyl OBD Column, 5 um, 19×250 mm; Mobile Phase A: Water (10 mmol/L NH₄HCO₃), Mobile Phase B: MeOH-HPLC; Flow rate: 25 mL/min; Gradient: 40% B to 63% B in 10 min; 254 nm) to afford N-[4-[(4-[imidazo[1,2-a]pyridin-7-yloxy]-3-methylphenyl)amino]quinazolin-6-yl]but-2-ynamide (Compound 2) (9.0 mg, 3%) as a white solid. LCMS (ESI, m/z): [M+H]⁺=449.2. ¹H NMR (300 MHz, DMSO-d₆): δ 10.94 (s, 1H), 9.87 (s, 1H), 8.77 (s, 1H), 8.56-8.54 (m, 2H), 7.84-7.73 (m, 5H), 7.44 (d, J=0.9 Hz, 1H), 7.13 (d, J=8.7 Hz, 1H), 6.82-6.79 (m, 1H), 6.56 (d, J=2.4 Hz, 1H), 2.20 (s, 3H), 2.09 (s, 3H).

### Example S3. Synthesis of N-[4-([3-methyl-4-[(1-methyl-2-oxopyridin-4-yl)oxy]phenyl]amino)quinazolin-6-yl]but-2-ynamide (Compound 3)

### Step 1: Synthesis of 1-methyl-4-(2-methyl-4-nitrophenoxy)pyridin-2-one

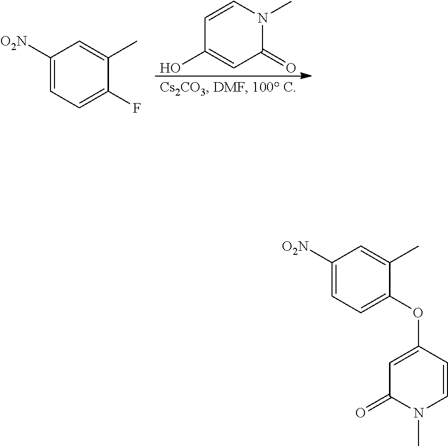

A mixture of 1-fluoro-2-methyl-4-nitrobenzene (1.0 g, 6.70 mmol), 4-hydroxy-1-methylpyridin-2-one (0.9 g, 7.11 mmol) and Cs₂CO₃ (4.4 g, 13.41 mmol) in DMF (20.0 mL) was stirred at 100 C for 4 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum to afford 1-methyl-4-(2-methyl-4-nitrophenoxy)pyridin-2-one (1.6 g, crude) as a yellow solid. LCMS (ESI, m/z): [M+H]⁺=261.0.

### Step 2: Synthesis of 4-(4-amino-2-methylphenoxy)-1-methylpyridin-2-one

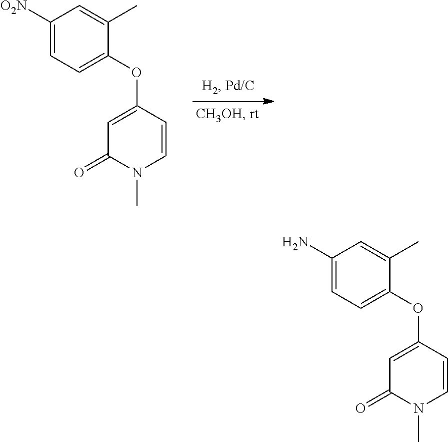 [SMILES: Cc1cc([N+](=O)[O-])ccc1Oc1ccn(C)c(=O)c1 ; Cc1cc(N)ccc1Oc1ccn(C)c(=O)c1]

A mixture of 1-methyl-4-(2-methyl-4-nitrophenoxy)pyridin-2-one (1.9 g, 5.99 mmol) and Pd/C (307.4 mg, dry) in CH₃OH (14.0 mL) was stirred at room temperature for 4 h under H₂. After the reaction was completed, the resulting mixture was filtered. The filtrate was concentrated under reduced pressure to afford 4-(4-amino-2-methylphenoxy)-1-methylpyridin-2-one (1.3 g, crude) as a brown yellow oil. LCMS (ESI, m/z): [M+H]⁺=231.1.

### Step 3: Synthesis of 1-methyl-4-[2-methyl-4-[(6-nitroquinazolin-4-yl)amino]phenoxy]pyridin-2-one

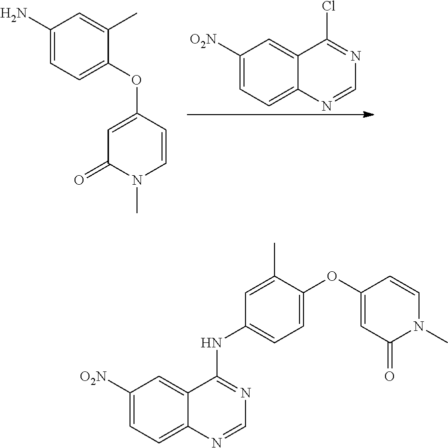 [SMILES: Cc1cc(N)ccc1Oc1ccn(C)c(=O)c1 ; O=[N+]([O-])c1ccc2ncnc(Cl)c2c1 ; Cc1cc(Nc2ncnc3ccc([N+](=O)[O-])cc23)ccc1Oc1ccn(C)c(=O)c1]

A mixture of 4-(4-amino-2-methylphenoxy)-1-methylpyridin-2-one (665.8 mg, 2.89 mmol), 4-chloro-6-nitroquinazoline (495.0 mg, 2.36 mmol) and DIEA (1.1 g, 8.53 mmol) in CH₃CH₂OH (15.0 mL) was stirred at 90 C for 2 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/MeOH (91/9, v/v) to afford 1-methyl-4-[2-methyl-4-[(6-nitroquinazolin-4-yl)amino]phenoxy]pyridin-2-one (600.0 mg, 62%) as a yellow solid. LCMS (ESI, m/z): [M+H]⁺=404.1.

### Step 4: Synthesis of 4-[4-[(6-aminoquinazolin-4-yl)amino]-2-methylphenoxy]-1-methylpyridin-2-one

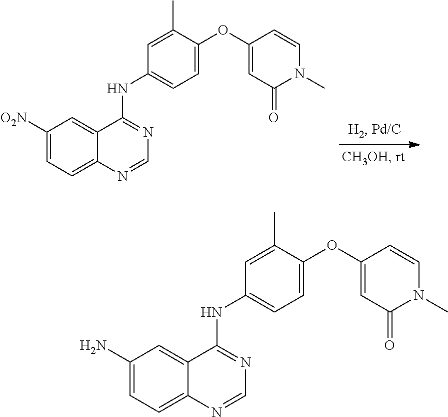 [SMILES: Cc1cc(Nc2ncnc3ccc([N+](=O)[O-])cc23)ccc1Oc1ccn(C)c(=O)c1 ; Cc1cc(Nc2ncnc3ccc(N)cc23)ccc1Oc1ccn(C)c(=O)c1]

A mixture of 1-methyl-4-[2-methyl-4-[(6-nitroquinazolin-4-yl)amino]phenoxy]pyridin-2-one (450.0 mg, 1.12 mmol) and Pd/C (150.8 mg, dry) in CH₃OH (12.0 mL) was stirred at room temperature for 4 h under H₂. After the reaction was completed, the resulting mixture was filtered. The filtrate was concentrated under reduced pressure to afford 4-[4-[(6-aminoquinazolin-4-yl)amino]-2-methylphenoxy]-1-methylpyridin-2-one (358.1 mg, crude) as a yellow solid. LCMS (ESI, m/z): [M+H]⁺=374.1.

### Step 5: Synthesis of N-[4-([3-methyl-4-[(1-methyl-2-oxopyridin-4-yl)oxy]phenyl]amino)quinazolin-6-yl]but-2-ynamide (Compound 3)

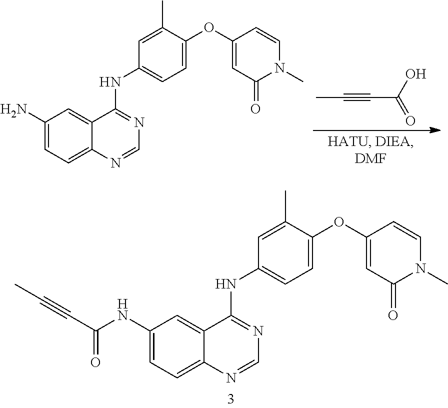

A mixture of 4-[4-[(6-aminoquinazolin-4-yl)amino]-2-methylphenoxy]-1-methylpyridin-2-one (500.0 mg, 1.34 mmol), 2-butynoic acid (135.1 mg, 1.61 mmol), HATU (1018.2 mg, 2.67 mmol) and DIEA (865.3 mg, 6.70 mmol) in DMF (15.0 mL) was stirred at 0° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (89/11, v/v) and then purified by Prep-HPLC with the following conditions (Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 19% B to 49% B in 10 min; 254 nm; retention time (RT) 1:9.60 min) to afford N-[4-([3-methyl-4-[(1-methyl-2-oxopyridin-4-yl)oxy]phenyl]amino)quinazolin-6-yl]but-2-ynamide (Compound 3) (28.5 mg, 4%) as a yellow solid. LCMS (ESI, m/z): [M+H]⁺=440.2. ¹H NMR (300 MHz, DMSO-d₆): δ 10.91 (s, 1H), 9.85 (s, 1H), 8.74 (s, 1H), 8.54 (s, 1H), 7.78-7.70 (m, 5H), 7.11 (d, J=8.7 Hz, 1H), 6.11-6.07 (m, 1H), 5.35 (d, J=2.4 Hz, 1H), 3.36 (s, 3H), 2.14 (s, 3H), 2.09 (s, 3H).

### Example S4. Synthesis of 4-(dimethylamino)-N-[4-[(3-methyl-4-[[1,2,4]triazolo[1,5-a]pyridin-7-yloxy]phenyl)amino]quinazolin-6-yl]but-2-ynamide (Compound 4)

### Step 1: Synthesis of 4-(dimethylamino)but-2-ynoic acid

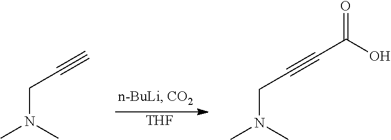

To a solution of dimethyl(prop-2-yn-1-yl)amine (1.0 g, 12.03 mmol) in THF (10.0 mL) was added dropwise n-BuLi (5.0 mL, 53.08 mmol) at −78° C. under N₂. The mixture was stirred at −78° C. for 1 h under N₂. Then dry carbon dioxide was pass through overnight at −78° C. After the reaction was completed, the reaction was quenched with H₂O and extracted with ethyl acetate. The combined organic layer was washed with brine, dried over anhydrous sodium sulfate and filtered. The filtrate was concentrated under vacuum. The residue was purified by reverse phase flash column chromatography with acetonitrile/water (1/9, v/v) to afford 4-(dimethylamino)but-2-ynoic acid (500.0 mg, 32%) as a yellow oil. LCMS (ESI, m/z): [M+H]⁺=128.1.

### Step 2: Synthesis of 4-(dimethylamino)-N-[4-[(3-methyl-4-[[1,2,4]triazolo[1,5-a]pyridin-7-yloxy]phenyl)amino]quinazolin-6-yl]but-2-ynamide (Compound 4)

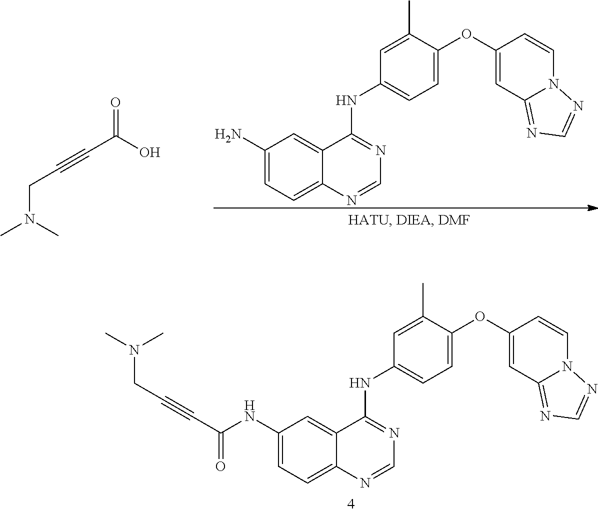

To a solution of N4-(3-methyl-4-[[1,2,4]triazolo[1,5-a]pyridin-7-yloxy]phenyl)quinazoline-4,6-diamine (170.0 mg, 0.44 mmol) in DMF (5.0 mL) was added 4-(dimethylamino)but-2-ynoic acid (281.9 mg, 2.22 mmol), DIEA (171.9 mg, 1.33 mmol) and HATU (252.9 mg, 0.66 mmol) at room temperature. The resulting mixture was stirred at room temperature for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with acetonitrile/water (2/3, v/v). and then purified by Prep-HPLC with the following conditions (Xselect CSH OBD Column 30×150 mm, 5 um; Mobile Phase A: Water (0.1% FA), Mobile Phase B: ACN; Flow rate: 60 mL/min; Gradient: 22% B to 46 B % in 7 min; 254 nm) to afford 4-(dimethylamino)-N-[4-[(3-methyl-4-[[1,2,4]triazolo[1,5-a]pyridin-7-yloxy]phenyl)amino]quinazolin-6-yl]but-2-ynamide (Compound 4) (12.3 mg, 5%) as a white solid. LCMS (ESI, m/z): [M+H]⁺=493.0. ¹H NMR (300 MHz, DMSO-d₆): δ 11.00 (s, 1H), 9.88 (s, 1H), 8.94 (d, J=7.8 Hz, 1H), 8.75 (s, 1H), 8.57 (s, 1H), 8.39 (s, 1H), 7.84-7.78 (m, 4H), 7.21 (d, J=6.0 Hz, 1H), 7.05-7.02 (m, 1H), 6.82 (s, 1H), 3.51 (s, 2H), 2.29 (s, 6H), 2.20 (s, 3H).

### Example S5. Synthesis of N-[4-([3-methyl-4-[(6-methylpyridin-3-yl)oxy]phenyl]amino)quinazolin-6-yl]but-2-ynamide (Compound 5)

### Step 1: Synthesis of 2-methyl-5-(2-methyl-4-nitrophenoxy)pyridine

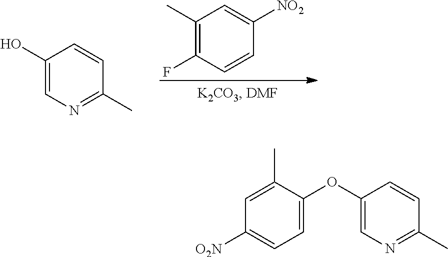

To a solution of 6-methylpyridin-3-ol (5.0 g, 45.82 mmol) in DMF (30.0 mL) was added 1-fluoro-2-methyl-4-nitrobenzene (8.5 g, 54.98 mmol) and K₂CO₃ (12.7 g, 91.64 mmol) at room temperature. The resulting mixture was stirred at 50° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse flash column chromatography with acetonitrile/water (55/45, v/v) to afford 2-methyl-5-(2-methyl-4-nitrophenoxy)pyridine (4.0 g, 35%) as a yellow oil. LCMS (ESI, m/z): [M+H]⁺=245.1.

### Step 2a: Synthesis of 3-methyl-4-[(6-methylpyridin-3-yl)oxy]aniline

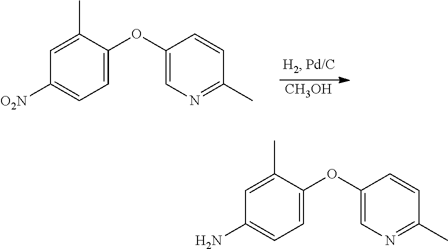

To a solution of 2-methyl-5-(2-methyl-4-nitrophenoxy)pyridine (4.0 g, 16.38 mmol) in CH₃OH (30.0 mL) was added Pd/C (1.7 g, dry) at room temperature. The resulting mixture was stirred at room temperature for 16 h under H₂. After the reaction was completed, the resulting mixture was filtered. The filtrate was concentrated under reduced pressure. The residue was purified by flash column chromatography with petroleum ether/ethyl acetate (36/64, v/v) to afford 3-methyl-4-[(6-methylpyridin-3-yl)oxy]aniline (3.5 g, 99%) as a white solid. LCMS (ESI, m/z): [M+H]⁺=215.1.

### Step 2b: Alternative Synthesis of 3-methyl-4-[(6-methylpyridin-3-yl)oxy]aniline

In an alternative to Step 2a: To a solution of 2-methyl-5-(2-methyl-4-nitrophenoxy)pyridine (30.10 g, 123.2 mmol) in MeOH (300.0 mL) was added Pd/C (3.0 g, dry) at 0° C. and was stirred for 19 h at room temperature under H₂ atmosphere. After the reaction was completed, the resulting mixture was filtered. The filtrate was concentrated under reduced pressure to afford 27.1 g (crude) of 3-methyl-4-((6-methylpyridin-3-yl)oxy)aniline as a yellow solid. LCMS (ESI, m/z): [M+H]⁺=215.

### Step 3a: Synthesis of N-[3-methyl-4-[(6-methylpyridin-3-yl)oxy]phenyl]-6-nitroquinazolin-4-amine

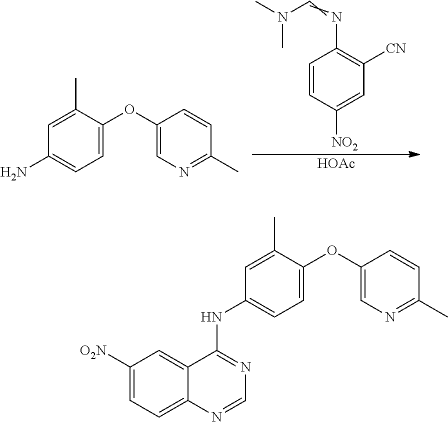 [SMILES: Cc1ccc(Oc2ccc(N)cc2C)cn1 ; CN(C)C=Nc1ccc([N+](=O)[O-])cc1C#N ; Cc1ccc(Oc2ccc(Nc3ncnc4ccc([N+](=O)[O-])cc34)cc2C)cn1 ; "HOAc"]

To a solution of 3-methyl-4-[(6-methylpyridin-3-yl)oxy]aniline ((2.0 g, 9.33 mmol) in HOAc (20.0 mL) was added N′-(2-cyano-4-nitrophenyl)-N,N-dimethylformimidamide (2.0 g, 9.33 mmol) at room temperature. The resulting mixture was stirred at 85° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (19/81, v/v) to afford N-[3-methyl-4-[(6-methylpyridin-3-yl)oxy]phenyl]-6-nitroquinazolin-4-amine (3.0 g, 82%) as a red solid. LCMS (ESI, m/z): [M+H]⁺=388.1.

### Step 3b: Alternative Synthesis of N-[3-methyl-4-[(6-methylpyridin-3-yl)oxy]phenyl]-6-nitroquinazolin-4-amine

In an alternative to Step 3a: To a solution of placed 3-methyl-4-[(6-methylpyridin-3-yl)oxy]aniline (12.00 g, 56.0 mmol) in HOAc (100.0 mL) was added N′-(2-cyano-4-nitrophenyl)-N,N-dimethylformimidamide (12.83 g, 58.8 mmol) at room temperature and was stirred for 2 h at 100° C. LCMs show reaction was completed. The mixture was cooled to room temperature and was diluted with of H2O. The resulting solution was extracted with ethyl acetate. The resulting mixture was washed with H2O and brine. The mixture was dried over anhydrous sodium sulfate and concentrated. The residue was applied onto a silica gel column with ethyl acetate/petroleum ether (8:2) to afford 17.9 g (82%) of N-(3-methyl-4-((6-methylpyridin-3-yl)oxy)phenyl)-6-nitroquinazolin-4-amine as a red solid. LCMS (ESI, m/z): [M+H]⁺=388.

### Step 4a: Synthesis of N4-[3-methyl-4-[(6-methylpyridin-3-yl)oxy]phenyl]quinazoline-4,6-diamine

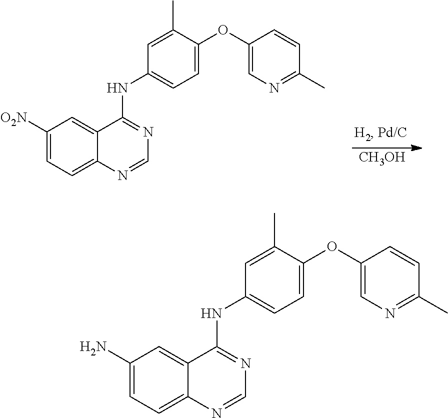 [SMILES: Cc1ccc(Oc2ccc(Nc3ncnc4ccc([N+](=O)[O-])cc34)cc2C)cn1 ; "CH3OH" ; Cc1ccc(Oc2ccc(Nc3ncnc4ccc(N)cc34)cc2C)cn1]

To a solution of N-[3-methyl-4-[(6-methylpyridin-3-yl)oxy]phenyl]-6-nitroquinazolin-4-amine (1.5 g, 3.87 mmol) in CH₃OH (10.0 mL) was added Pd/C (412.1 mg, dry) at room temperature. The resulting mixture was stirred at room temperature for 2 h under H₂. After the reaction was completed, the resulting mixture was filtered. The filtrate was concentrated under reduced pressure. The residue was purified by flash column chromatography with CH₂Cl₂/CH₃OH (86/14, v/v) to afford N4-[3-methyl-4-[(6-methylpyridin-3-yl)oxy]phenyl]quinazoline-4,6-diamine (800.0 mg, 57%) as a yellow solid. LCMS (ESI, m/z): [M+H]⁺=358.2.

### Step 4b: Alternative Synthesis of N4-[3-methyl-4-[(6-methylpyridin-3-yl)oxy]phenyl]quinazoline-4,6-diamine

In an alternative to Step 4a: To a solution of N-(3-methyl-4-((6-methylpyridin-3-yl)oxy)phenyl)-6-nitroquinazolin-4-amine (14.00 g, 36.14 mmol) in MeOH (200.0 mL) was added Pd/C (2.00 g, dry) and was stirred for 18 h at room temperature. After the reaction was completed, the resulting mixture was filtered. The filtrate was concentrated under reduced pressure. The residue was applied onto a silica gel column with dichloromethane/methanol (92:8) to afford 10.7 g (82%) of N4-(3-methyl-4-((6-methylpyridin-3-yl)oxy)phenyl)quinazoline-4,6-diamine as a yellow solid. LCMS (ESI, m/z): [M+H]⁺=358.

### Step 5a: Synthesis of N-[4-([3-methyl-4-[(6-methylpyridin-3-yl)oxy]phenyl]amino)quinazolin-6-yl]but-2-ynamide (Compound 5)

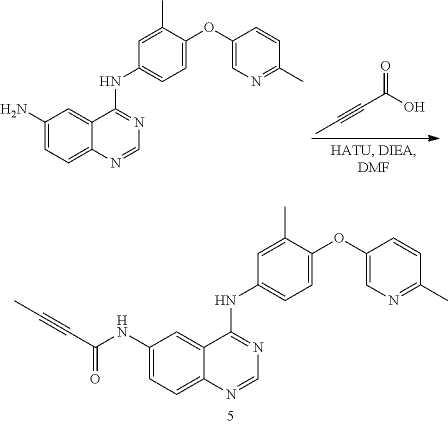

To a mixture of N4-[3-methyl-4-[(6-methylpyridin-3-yl)oxy]phenyl]quinazoline-4,6-diamine (800.0 mg, 2.24 mmol) and 2-butynoic acid (244.6 mg, 2.91 mmol) in DMF (5.0 mL) was added DIEA (2.9 g, 22.38 mmol) and HATU (1.4 g, 3.58 mmol) at room temperature. The resulting mixture was stirred at room temperature for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with acetonitrile/water (68/32, v/v) and then purified by Prep-HPLC with the following conditions (Column, 19×250 mm, 5 um; Mobile Phase A: Water (10 mmol/L NH₄HCO₃), Mobile Phase B: MeOH; Preparative; Flow rate: 25 mL/min; Gradient: 64% B to 74% B in 9 min; 254 nm) to afford N-[4-([3-methyl-4-[(6-methylpyridin-3-yl)oxy]phenyl]amino)quinazolin-6-yl]but-2-ynamide (Compound 5) (128.6 mg, 13%) as a white solid. LCMS (ESI, m/z): [M+H]⁺=424.2. ¹H NMR (300 MHz, DMSO-d₆): δ 10.91 (s, 1H), 9.80 (s, 1H), 8.74 (s, 1H), 8.52 (s, 1H), 8.19 (d, J=1.8 Hz, 1H), 7.81-7.74 (m, 3H), 7.67-7.64 (m, 1H), 7.27-7.19 (m, 2H), 6.96 (d, J=8.7 Hz, 1H), 2.39 (s, 3H), 2.21 (s, 3H), 2.09 (s, 3H).

### Step 5b: Alternative Synthesis of N-[4-([3-methyl-4-[(6-methylpyridin-3-yl)oxy]phenyl]amino)quinazolin-6-yl]but-2-ynamide (Compound 5)

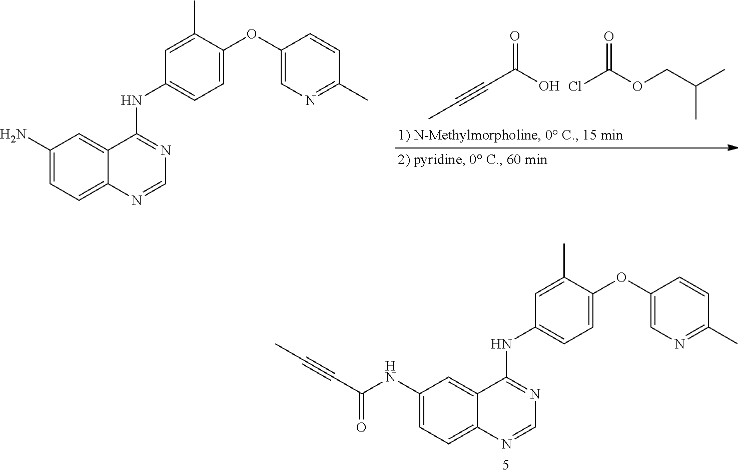

In an alternative to Step 5a: Under N2 atmosphere, to a solution of but-2-ynoic acid (1.41 g, 16.78 mmol) in THF (50.0 mL) was dropped wise isobutyl carbonochloridate (2.67 g, 19.59 mmol) at 0° C. and was stirred for 5 min at 0° C. Then N-methylmorpholine (2.26 g, 22.38 mmol) was dropped wise at 0° C. and was stirred for 10 min at 0° C. Then a solution of N4-[3-methyl-4-[(6-methylpyridin-3-yl)oxy]phenyl]quinazoline-4,6-diamine (2.00 g, 5.596 mmol) in pyridine (5.0 mL) was dropped wise at 0° C. and was stirred for 5 min at 0° C. LCMS show reaction was completed. The reaction was then quenched by the addition of water. The resulting solution was extracted with ethyl acetate. The resulting mixture was washed with brine. The mixture was dried over anhydrous sodium sulfate and concentrated. The residue was applied onto a silica gel column with dichloromethane/methanol (95:5) to afford 2.1 g crude product. The 2.1 g crude product was purified by reverse flash column chromatography with water (0.05% NH₄HCO₃ in H₂O)/MeOH (40:60) to afford 1.5510 g (65%) of N-[4-([3-methyl-4-[(6-methylpyridin-3-yl)oxy]phenyl]amino)quinazolin-6-yl]but-2-ynamide (Compound 5) as a solid. LCMS (ESI, m/z): [M+H]⁺=424. ¹H NMR (300 MHz, DMSO-d₆) δ 10.92 (s, 1H), 9.81 (s, 1H), 8.74 (s, 1H), 8.51 (s, 1H), 8.19-8.17 (m, 1H), 7.80-7.77 (m, 3H), 7.67-7.62 (m, 1H), 7.25-7.17 (m, 2H), 6.95 (d, J=8.7 Hz, 1H), 2.43 (s, 3H), 2.20 (s, 3H), 2.08 (s, 3H).

### Example S6. Synthesis of N-[4-[(4-[[1,2,4]triazolo[1,5-a]pyridin-6-yloxy]phenyl)amino]quinazolin-6-yl]but-2-ynamide (Compound 6)

### Step 1: Synthesis of 5-(4-nitrophenoxy)pyridin-2-amine

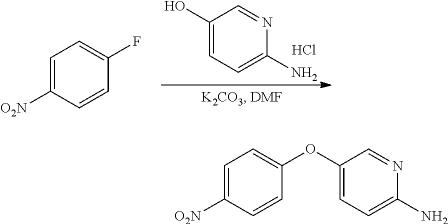 [SMILES: O=[N+]([O-])c1ccc(F)cc1 ; Nc1ccc(O)cn1 ; Cl ; Nc1ccc(Oc2ccc([N+](=O)[O-])cc2)cn1]

To a mixture of 4-fluoronitrobenzene (1000.0 mg, 7.09 mmol) and K₂CO₃ (2484.0 mg, 17.97 mmol) in DMF (15.0 mL) was added 6-aminopyridin-3-ol (660.0 mg, 5.99 mmol). The resulting mixture was stirred at 50° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford 5-(4-nitrophenoxy)pyridin-2-amine (730.0 mg, 53%) as a yellow solid. LCMS (ESI, m/z): [M+H]⁺=232.1.

### Step 2: Synthesis of (Z)—N,N-dimethyl-N′-[5-(4-nitrophenoxy)pyridin-2-yl]methanimidamide

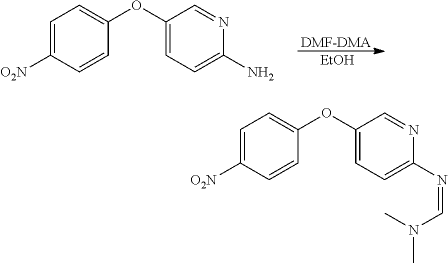

A mixture of 5-(4-nitrophenoxy)pyridin-2-amine (730.0 mg, 3.16 mmol) and DMF-DMA (564.3 mg, 4.74 mmol) in EtOH (10.0 mL) was stirred at 70° C. for 3 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum to afford (Z)—N,N-dimethyl-N′-[5-(4-nitrophenoxy)pyridin-2-yl]methanimidamide (765.0 mg, crude) as a yellow solid. LCMS (ESI, m/z): [M+H]⁺=287.1.

### Step 3: Synthesis of (Z)—N-hydroxy-N′-[5-(4-nitrophenoxy)pyridin-2-yl]methanimidamide

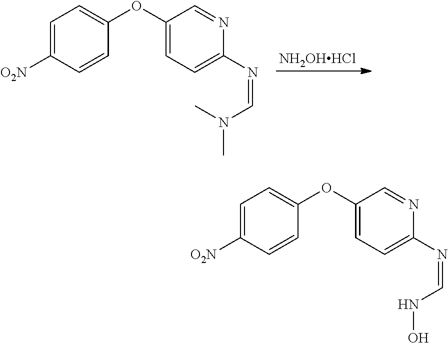

A solution of (Z)—N,N-dimethyl-N′-[5-(4-nitrophenoxy)pyridin-2-yl]methanimidamide (6.0 g, 20.96 mmol) and NH₂OH·HCl (5.1 g, 73.35 mmol) in CH₃CH₂OH (100.0 mL) was stirred at 50° C. for 3 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum to afford (Z)—N-hydroxy-N-[5-(4-nitrophenoxy)pyridin-2-yl]methanimidamide (4.3 g, crude) as a yellow solid. LCMS (ESI, m/z): [M+H]⁺=275.1.

### Step 4: Synthesis of 6-(4-nitrophenoxy)-[1,2,4]triazolo[1,5-a]pyridine

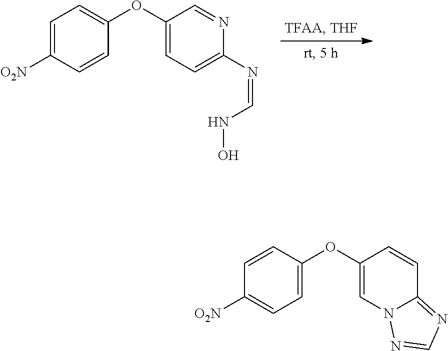 [SMILES: O=[N+]([O-])c1ccc(Oc2ccc(/N=C\NO)nc2)cc1 ; O=[N+]([O-])c1ccc(Oc2ccc3ncnn3c2)cc1]

A solution of (Z)—N-hydroxy-N′-[5-(4-nitrophenoxy)pyridin-2-yl]methanimidamide (2.0 g, 7.29 mmol) and TFAA (1.7 g, 8.02 mmol) in THF (30.0 mL) was stirred for at room temperature 5 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum to afford 6-(4-nitrophenoxy)-[1,2,4]triazolo[1,5-a]pyridine (1.8 g, crude) as a yellow solid. LCMS (ESI, m/z): [M+H]⁺=257.1.

### Step 5: Synthesis of 4-[[1,2,4]triazolo[1,5-a]pyridin-6-yloxy]aniline

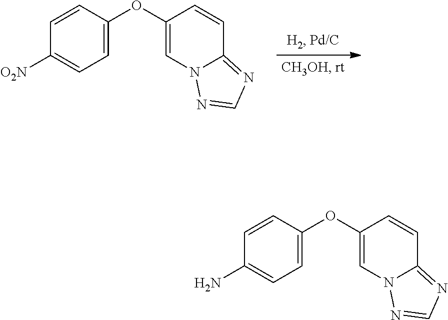 [SMILES: O=[N+]([O-])c1ccc(Oc2ccc3ncnn3c2)cc1 ; Nc1ccc(Oc2ccc3ncnn3c2)cc1]

To a solution of 6-(4-nitrophenoxy)-[1,2,4]triazolo[1,5-a]pyridine (3.0 g, 11.71 mmol) in CH₃OH (40.0 mL) was added Pd/C (1.0 g, dry) at room temperature. The mixture was stirred at room temperature for 3 h under H₂. After the reaction was completed, the resulting mixture was filtered. The filtrate was concentrated under vacuum. The residue was purified by flash column chromatography with CH₂Cl₂/CH₃OH (91/9, v/v) to afford 4-[[1,2,4]triazolo[1,5-a]pyridin-6-yloxy]aniline (2.3 g, 85%) as a yellow oil. LCMS (ESI, m/z): [M+H]⁺=227.1.

### Step 6: Synthesis of 6-nitro-N-(4-[[1,2,4]triazolo[1,5-a]pyridin-6-yloxy]phenyl)quinazolin-4-amine

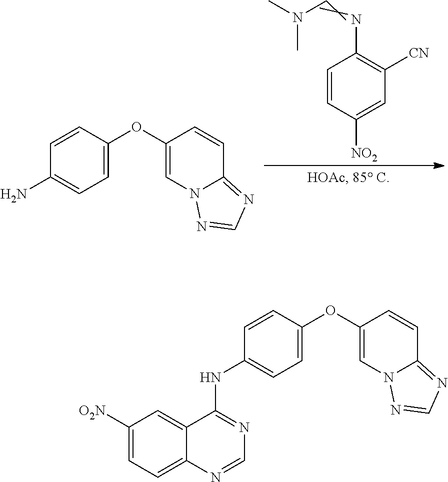

A mixture of 4-[[1,2,4]triazolo[1,5-a]pyridin-6-yloxy]aniline (500.0 mg, 2.21 mmol) and N′-(2-cyano-4-nitrophenyl)-N,N-dimethylformimidamide (482.3 mg, 2.21 mmol) in HOAc (10.0 mL) was stirred at 85° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford 6-nitro-N-(4-[[1,2,4]triazolo[1,5-a]pyridin-6-yloxy]phenyl)quinazolin-4-amine (616.0 mg, 70%) as a yellow solid. LCMS (ESI, m/z): [M+H]⁺=400.1.

### Step 7: Synthesis of N4-(4-[[1,2,4]triazolo[1,5-a]pyridin-6-yloxy]phenyl)quinazoline-4,6-diamine

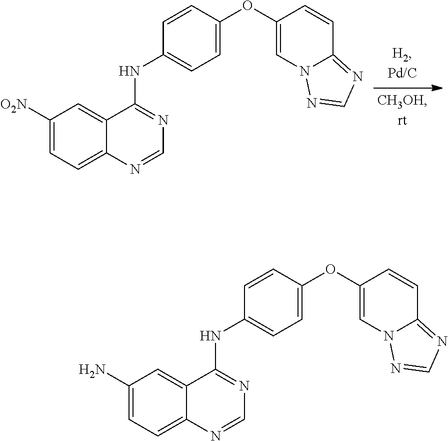 [SMILES: O=[N+]([O-])c1ccc2ncnc(Nc3ccc(Oc4ccc5ncnn5c4)cc3)c2c1 ; Nc1ccc2ncnc(Nc3ccc(Oc4ccc5ncnn5c4)cc3)c2c1]

To a solution of 6-nitro-N-(4-[[1,2,4]triazolo[1,5-a]pyridin-6-yloxy]phenyl)quinazolin-4-amine (560.0 mg, 1.40 mmol) in CH₃OH (15.0 mL) was added Pd/C (246.2 mg, dry). The mixture was stirred at room temperature for 16 h under H₂. After the reaction was completed, the resulting mixture was filtered. The filtrate was concentrated under vacuum to afford N4-(4-[[1,2,4]triazolo[1,5-a]pyridin-6-yloxy]phenyl)quinazoline-4,6-diamine (463.0 mg, crude). LCMS (ESI, m/z): [M+H]⁺=370.1.

### Step 8: Synthesis of N-[4-[(4-[[1,2,4]triazolo[1,5-a]pyridin-6-yloxy]phenyl)amino]quinazolin-6-yl]but-2-ynamide (Compound 6)

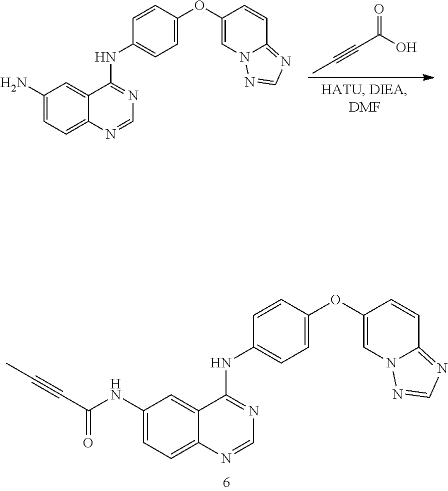

A mixture of N4-(4-[[1,2,4]triazolo[1,5-a]pyridin-6-yloxy]phenyl)quinazoline-4,6-diamine (440.0 mg, 1.19 mmol), 2-butynoic acid (200.3 mg, 2.38 mmol), DIEA (461.9 mg, 3.57 mmol) and HATU (1358.7 mg, 3.57 mmol) in DMF (20.0 mL) was stirred at 0° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) and then purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 9% B to 30% B in 7 min; 254/220 nm) to afford N-[4-[(4-[[1,2,4]triazolo[1,5-a]pyridin-6-yloxy]phenyl)amino]quinazolin-6-yl]but-2-ynamide (Compound 6) (51.8 mg, 10%) as a white solid. LCMS (ESI, m/z): [M+H]⁺=436.2. ¹H NMR (400 MHz, DMSO-d₆): δ 10.92 (s, 1H), 9.86 (s, 1H), 8.91 (s, 1H), 8.73 (s, 1H), 8.51 (d, J=5.2 Hz, 2H), 7.93-7.90 (m, 1H), 7.83-7.75 (m, 4H), 7.62-7.59 (m, 1H), 7.18-7.14 (m, 2H), 2.12 (s, 3H).

### Example S7. Synthesis of N-(4-((4-((1-(difluoromethyl)-2-oxo-1,2-dihydropyridin-4-yl)oxy)phenyl)amino)quinazolin-6-yl)but-2-ynamide (Compound 7)

### Step 1: Synthesis of 2-chloro-4-(4-nitrophenoxy)pyridine

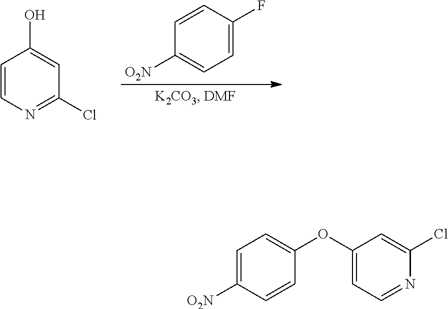 [SMILES: Oc1ccnc(Cl)c1 ; O=[N+]([O-])c1ccc(F)cc1 ; O=[N+]([O-])c1ccc(Oc2ccnc(Cl)c2)cc1]

A mixture of 2-chloropyridin-4-ol (3.0 g, 23.15 mmol), 4-fluoronitrobenzene (3.9 g, 27.79 mmol) and K₂CO₃ (6.4 g, 46.31 mmol) in DMF (30.0 mL) was stirred at 50° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (7/3, v/v) to afford 2-chloro-4-(4-nitrophenoxy)pyridine (4.3 g, 74%) as a light yellow solid. LCMS (ESI, m/z): [M+H]⁺=251.0.

### Step 2: Synthesis of 1-(difluoromethyl)-4-(4-nitrophenoxy)pyridin-2-one

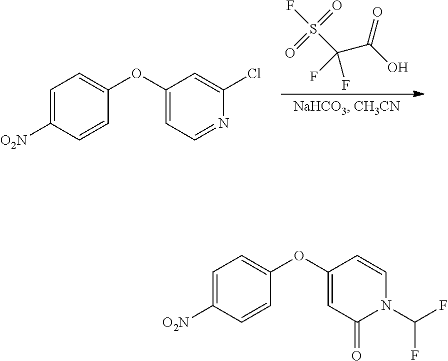

To a solution of 2-chloro-4-(4-nitrophenoxy)pyridine (3.1 g, 12.56 mmol) in CH₃CN (20.0 mL) was added NaHCO₃ (2.1 g, 25.11 mmol). The resulting mixture was heated at 80° C. for 30 minutes. A solution of 2,2-difluoro-2-(fluorosulfonyl)acetic acid (2.2 g, 12.56 mmol) in CH₃CN (5.00 mL) was added to the mixture over 10 minutes. The reaction mixture was heated at 80° C. for another 2 h. After the reaction was completed, the resulting mixture was concentrated under vacuum. The residue was purified by flash column chromatography with petroleum ether/ethyl acetate (7/3, v/v) to afford 1-(difluoromethyl)-4-(4-nitrophenoxy)pyridin-2-one (1.4 g, 40%) as a light yellow solid. LCMS (ESI, m/z): [M+H]⁺=283.0.

### Step 3: Synthesis of 4-(4-aminophenoxy)-1-(difluoromethyl)pyridin-2(1H)-one

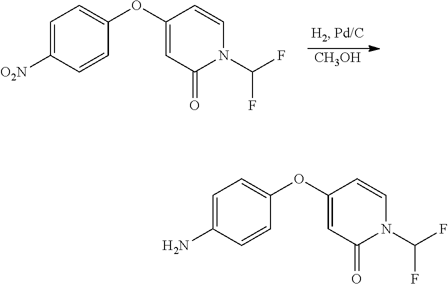

To a solution of 1-(difluoromethyl)-4-(4-nitrophenoxy)pyridin-2-one (1.3 g, 4.78 mmol) in CH₃OH (20.0 mL) was added Pd/C (448.0 mg, dry). The resulting mixture was stirred at room temperature for 2 h under H₂. After the reaction was completed, the resulting mixture was filtered. The filtrate was concentrated under reduced pressure to afford 4-(4-aminophenoxy)-1-(difluoromethyl)pyridin-2(1H)-one (996.0 mg, crude) as a yellow solid. LCMS (ESI, m/z): [M+H]⁺=253.0.

### Step 4: Synthesis of 1-(difluoromethyl)-4-[4-[(6-nitroquinazolin-4-yl)amino]phenoxy]pyridin-2-one

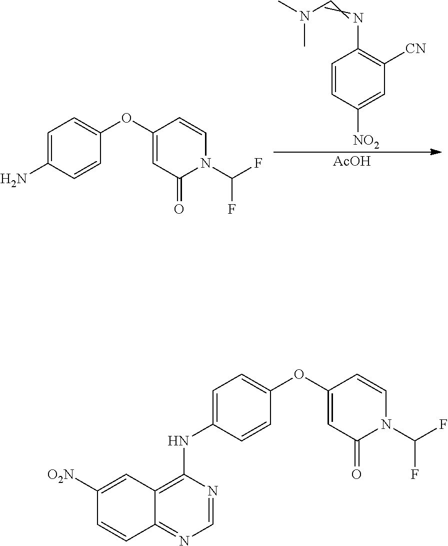

A mixture of 4-(4-aminophenoxy)-1-(difluoromethyl)pyridin-2(1H)-one (960.0 mg, 3.80 mmol) and N′-(2-cyano-4-nitrophenyl)-N,N-dimethylformimidamide (830.5 mg, 3.80 mmol) in HOAc (20.0 mL) was stirred at 85° C. for 2 h. After the reaction was completed, the mixture was concentrated under reduced pressure. The residue was purified by flash column chromatography with CH₂Cl₂/MeOH (10/1, v/v) to afford 1-(difluoromethyl)-4-[4-[(6-nitroquinazolin-4-yl)amino]phenoxy]pyridin-2-one (900.0 mg, 33%) as a yellow solid. LCMS (ESI, m/z): [M+H]⁺=426.1.

### Step 5: Synthesis of 4-[4-[(6-aminoquinazolin-4-yl)amino]phenoxy]-1-(difluoromethyl)pyridin-2-one

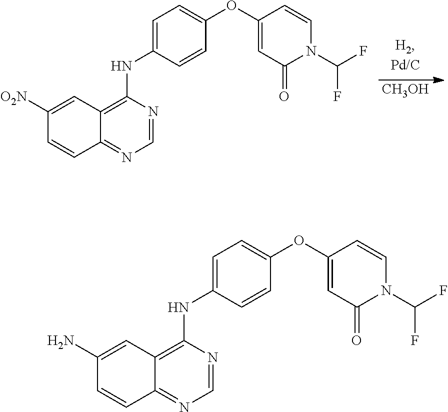

To a solution of 1-(difluoromethyl)-4-[4-[(6-nitroquinazolin-4-yl)amino]phenoxy]pyridin-2-one (900.0 mg, 2.11 mmol) in CH₃OH (40.0 mL) was added Pd/C (300.0 mg, dry) at room temperature. The resulting mixture was stirred at room temperature for 3 h under H₂. After the reaction was completed, the resulting mixture was filtered. The filtrate was concentrated under reduced pressure to afford 4-[4-[(6-aminoquinazolin-4-yl)amino]phenoxy]-1-(difluoromethyl)pyridin-2-one (560.0 mg, crude) as a yellow solid. LCMS (ESI, m/z): [M+H]⁺=396.1.

### Step 6: Synthesis of N-(4-((4-((1-(difluoromethyl)-2-oxo-1,2-dihydropyridin-4-yl)oxy)phenyl)amino)quinazolin-6-yl)but-2-ynamide (Compound 7)

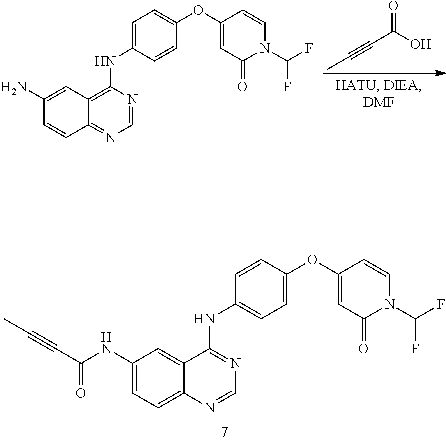

To a solution of 4-[4-[(6-aminoquinazolin-4-yl)amino]phenoxy]-1-(difluoromethyl)pyridin-2-one (500.0 mg, 1.26 mmol), 2-butynoic acid (159.4 mg, 1.89 mmol) and DIEA (490.3 mg, 3.79 mmol) in DMF (10.0 mL) was added HATU (721.2 mg, 1.89 mmol) at room temperature. The resulting mixture was stirred at room temperature for 2 h. After the reaction was completed, the resulting mixture was diluted with water and extracted with EtOAc. The combined organic layer was washed with brine, dried over anhydrous Na₂SO₄ and filtered. The filtrate was concentrated under reduced pressure. The residue was purified by flash column chromatography with CH₂Cl₂/CH₃OH (10/1, v/v) and then purified by Prep-HPLC with the following conditions (Column: YMC-Actus Triart C18, 20×250 mm, 5 um; Mobile Phase A: Water (10 mmol/L NH₄HCO₃), Mobile Phase B: ACN; Flow rate: 60 mL/min; Gradient: 28% B to 58% B in 10 min; 254 nm) to afford N-[4-[(4-[[1-(difluoromethyl)-2-oxopyridin-4-yl]oxy]phenyl)amino]quinazolin-6-yl]but-2-ynamide (Compound 7) (54.1 mg, 9%) as a white solid. LCMS (ESI, m/z): [M+H]⁺=462.1. ¹H NMR (300 MHz, DMSO-d₆): δ 10.94 (s, 1H), 9.94 (s, 1H), 8.75 (s, 1H), 8.55 (s, 1H), 8.01-7.61 (m, 6H), 7.26 (d, J=9.0 Hz, 2H), 6.38-6.35 (m, 1H), 5.51 (d, J=2.4 Hz, 1H), 2.10 (s, 3H).

### Example S8. Synthesis of N-methyl-N-[4-[(4-[[1,2,4]triazolo[1,5-a]pyridin-7-yloxy]phenyl)amino]quinazolin-6-yl]but-2-ynamide (Compound 8)

### Step 1: Synthesis of 4-(4-nitrophenoxy)pyridin-2-amine

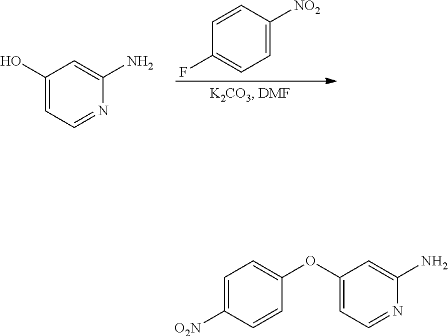 [SMILES: Nc1cc(O)ccn1 ; O=[N+]([O-])c1ccc(F)cc1 ; Nc1cc(Oc2ccc([N+](=O)[O-])cc2)ccn1]

To a solution of 2-aminopyridin-4-ol (1.0 g, 9.08 mmol) in DMF (10.0 mL) was added K₂CO₃ (2.5 g, 18.16 mmol) and 4-fluoronitrobenzene (1.54 g, 10.90 mmol) at room temperature. The resulting mixture was stirred at 50° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse flash column chromatography with acetonitrile/water (55/45, v/v) to afford 4-(4-nitrophenoxy)pyridin-2-amine (1.9 g, 90%) as a yellow solid. LCMS (ESI, m/z): [M+H]⁺=232.0.

### Step 2: Synthesis of (E)-N-hydroxy-N′-(4-(4-nitrophenoxy)pyridin-2-yl)formimidamide

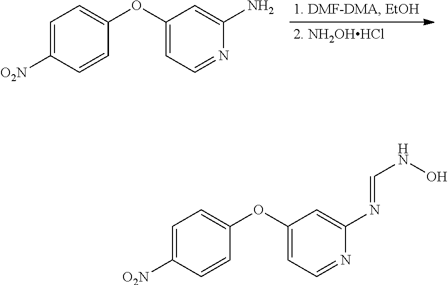 [SMILES: Nc1cc(Oc2ccc([N+](=O)[O-])cc2)ccn1 ; O=[N+]([O-])c1ccc(Oc2ccnc(/N=C/NO)c2)cc1]

To a solution of 4-(4-nitrophenoxy)pyridin-2-amine (1.9 g, 8.22 mmol) in EtOH (20.0 mL) was added DMF-DMA (1.7 g, 13.97 mmol) at room temperature. The resulting mixture was stirred at 75° C. for 3 h. Then hydroxylamine hydrochloride (2.0 g, 28.76 mmol) was added to the mixture at 40° C. The resulting mixture was stirred at 50° C. for another 1 h. After the reaction was completed, the reaction mixture was cooled to room temperature and then filtered. The solid was washed with EtOH and dried to afford (E)-N-hydroxy-N′-(4-(4-nitrophenoxy)pyridin-2-yl)formimidamide (1.8 g, crude) as a white solid. LCMS (ESI, m/z): [M+H]⁺=275.0.

### Step 3: Synthesis of 7-(4-nitrophenoxy)-[1,2,4]triazolo[1,5-a]pyridine

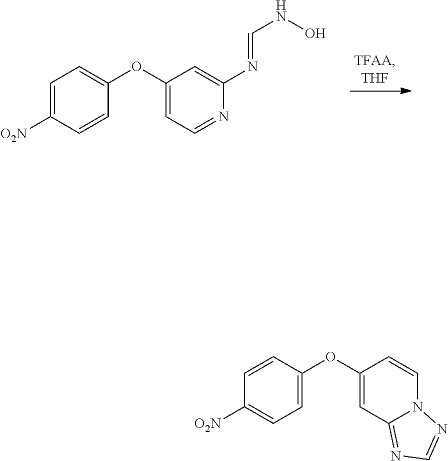 [SMILES: O=[N+]([O-])c1ccc(Oc2ccnc(/N=C/NO)c2)cc1 ; O=[N+]([O-])c1ccc(Oc2ccn3ncnc3c2)cc1]

To a solution of (E)-N-hydroxy-N′-(4-(4-nitrophenoxy)pyridin-2-yl)formimidamide (1.8 g, 6.56 mmol) in THF (20.0 mL) was added TFAA (1.5 g, 7.22 mmol) at room temperature. The resulting mixture was stirred at room temperature for 5 h. After the reaction was completed, the resulting mixture was concentrated under reduced pressure to afford 7-(4-nitrophenoxy)-[1,2,4]triazolo[1,5-a]pyridine (1.5 g, crude) as a light yellow solid. LCMS (ESI, m/z): [M+H]⁺=257.0.

### Step 4: Synthesis of 4-[[1,2,4]triazolo[1,5-a]pyridin-7-yloxy]aniline

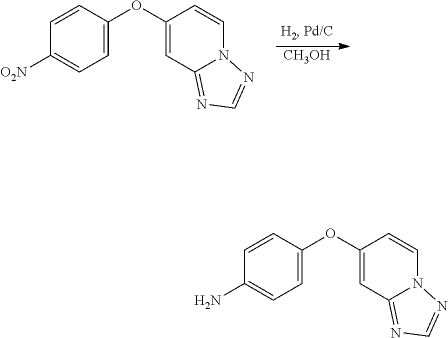

To a solution of 7-(4-nitrophenoxy)-[1,2,4]triazolo[1,5-a]pyridine (1.5 g, 5.85 mmol) in CH₃OH (10.0 mL) was added Pd/C (623.0 mg, dry) at room temperature. The resulting mixture was stirred at room temperature for 2 h under H₂. After the reaction was completed, the resulting mixture was filtered. The filtrate was concentrated under reduced pressure. The residue was purified by flash column chromatography with CH₂Cl₂/CH₃OH (82/18, v/v) to afford 4-[[1,2,4]triazolo[1,5-a]pyridin-7-yloxy]aniline (1.0 g, 75%) as a yellow solid. LCMS (ESI, m/z): [M+H]⁺=227.1.

### Step 5: Synthesis of 6-nitro-N-(4-[[1,2,4]triazolo[1,5-a]pyridin-7-yloxy]phenyl)quinazolin-4-amine

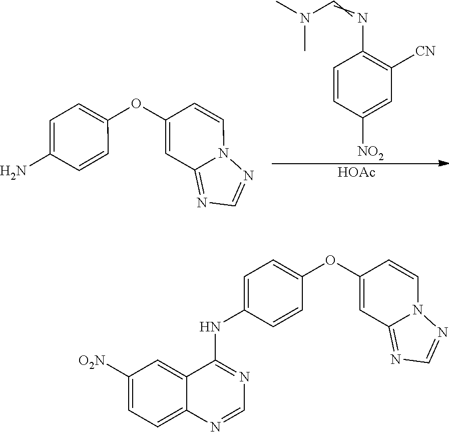 [SMILES: Nc1ccc(Oc2ccn3ncnc3c2)cc1 ; CN(C)C=Nc1ccc([N+](=O)[O-])cc1C#N ; O=[N+]([O-])c1ccc2ncnc(Nc3ccc(Oc4ccn5ncnc5c4)cc3)c2c1 ; "HOAc"]

To a solution of 4-[[1,2,4]triazolo[1,5-a]pyridin-7-yloxy]aniline (500.0 mg, 2.21 mmol) in HOAc (5.0 mL) was added N′-(2-cyano-4-nitrophenyl)-N,N-dimethylformimidamide (578.7 mg, 2.65 mmol) at room temperature. The resulting mixture was stirred at 85° C. for 2 h. After the reaction was completed, the resulting mixture was concentrated under reduced pressure. The residue was purified by flash column chromatography with CH₂Cl₂/CH₃OH (12/1, v/v) to afford 6-nitro-N-(4-[[1,2,4]triazolo[1,5-a]pyridin-7-yloxy]phenyl)quinazolin-4-amine (375.0 mg, 42%) as a yellow solid. LCMS (ESI, m/z): [M+H]⁺=400.1.

### Step 6: Synthesis of N4-(4-[[1,2,4]triazolo[1,5-a]pyridin-7-yloxy]phenyl)quinazoline-4,6-diamine

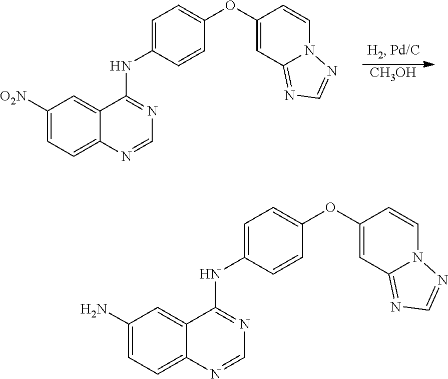

To a solution of 6-nitro-N-(4-[[1,2,4]triazolo[1,5-a]pyridin-7-yloxy]phenyl)quinazolin-4-amine (320.0 mg, 0.80 mmol) in CH₃OH (15.0 mL) was added Pd/C (191.9 mg, dry) at room temperature under N₂. The resulting mixture was stirred at room temperature for 3 h under H₂. After the reaction was completed, the resulting mixture was filtered. The filtrate was concentrated under reduced pressure. The residue was purified by flash column chromatography with CH₂Cl₂/CH₃OH (9/1, v/v) to afford N4-(4-[[1,2,4]triazolo[1,5-a]pyridin-7-yloxy]phenyl)quinazoline-4,6-diamine (280.0 mg, 94%) as a yellow solid. LCMS (ESI, m/z): [M+H]⁺=370.1.

### Step 7: Synthesis of N-[4-[(4-[[1,2,4]triazolo[1,5-a]pyridin-7-yloxy]phenyl)amino]quinazolin-6-yl]but-2-ynamide

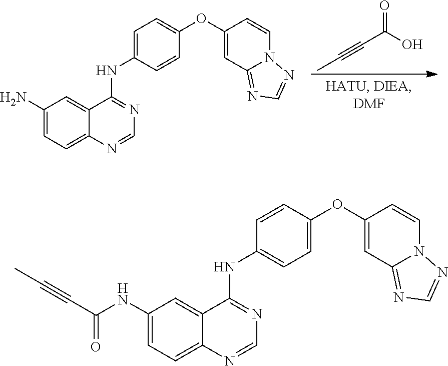

To a solution of N4-(4-[[1,2,4]triazolo[1,5-a]pyridin-7-yloxy]phenyl)quinazoline-4,6-diamine (300.0 mg, 0.81 mmol) in DMF (5.0 mL) was added 2-butynoic acid (88.8 mg, 1.06 mmol), DIEA (1.05 g, 8.12 mmol) and HATU (494.1 mg, 1.30 mmol) at room temperature. The resulting mixture was stirred at room temperature for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with acetonitrile/water (59/41, v/v) to afford N-[4-[(4-[[1,2,4]triazolo[1,5-a]pyridin-7-yloxy]phenyl)amino]quinazolin-6-yl]but-2-ynamide (100.0 mg, 28%) as a yellow solid. LCMS (ESI, m/z): [M+H]⁺=436.1.

### Step 8: Synthesis of N-methyl-N-[4-[(4-[[1,2,4]triazolo[1,5-a]pyridin-7-yloxy]phenyl)amino]quinazolin-6-yl]but-2-ynamide (Compound 8)

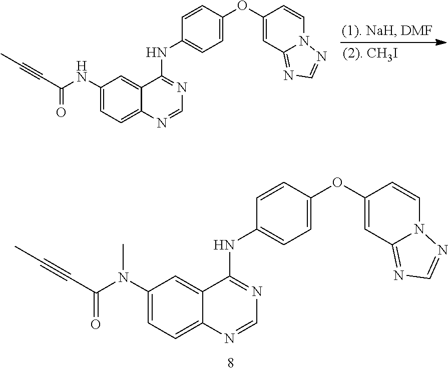 [SMILES: CC#CC(=O)Nc1ccc2ncnc(Nc3ccc(Oc4ccn5ncnc5c4)cc3)c2c1 ; CC#CC(=O)N(C)c1ccc2ncnc(Nc3ccc(Oc4ccn5ncnc5c4)cc3)c2c1]

To a solution of N-[4-[(4-[[1,2,4]triazolo[1,5-a]pyridin-7-yloxy]phenyl)amino]quinazolin-6-yl]but-2-ynamide (100.0 mg, 0.23 mmol) in DMF (5.0 mL) was added NaH (27.6 mg, 60%) at 0° C. The resulting mixture was stirred at 0° C. for 30 min. Then CH₃I (32.6 mg, 0.23 mmol) was added to the mixture at 0° C. The resulting mixture was stirred at room temperature for another 1 h. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with CH₃OH/water (67/33, v/v) and then purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 7% B to 37% B in 7 min; 254 nm) to afford N-methyl-N-[4-[(4-[[1,2,4]triazolo[1,5-a]pyridin-7-yloxy]phenyl)amino]quinazolin-6-yl]but-2-ynamide (Compound 8) (2.3 mg, 2%) as a white solid. LCMS (ESI, m/z): [M+H]⁺=450.2. ¹H NMR (300 MHz, DMSO-d₆): δ 9.95 (s, 1H), 8.95 (d, J=8.4 Hz, 1H), 8.65 (s, 2H), 8.41 (s, 1H), 8.01-7.77 (m, 4H), 7.31 (d, J=9.0 Hz, 2H), 7.06-7.03 (m, 2H), 3.35 (s, 3H), 2.15 (s, 1H), 1.74 (s, 2H).

### Example S9. Synthesis of N-[4-([4-[(6-methylpyridin-3-yl)oxy]phenyl]amino)quinazolin-6-yl]but-2-ynamide (Compound 9)

### Step 1: Synthesis of 2-methyl-5-(4-nitrophenoxy)pyridine

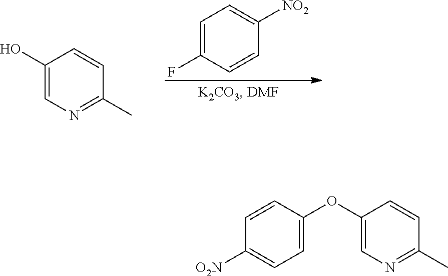

To a solution of 6-methylpyridin-3-ol (1.0 g, 9.16 mmol) in DMF (10.0 mL) was added K₂CO₃ (2.5 g, 18.33 mmol) and 4-fluoronitrobenzene (1.6 g, 10.99 mmol) at room temperature. The resulting mixture was stirred at 50° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with acetonitrile/water (45/55, v/v) to afford 2-methyl-5-(4-nitrophenoxy)pyridine (1.5 g, 71%) as a yellow solid. LCMS (ESI, m/z): [M+H]⁺=231.0.

### Step 2: Synthesis of 4-[(6-methylpyridin-3-yl)oxy]aniline

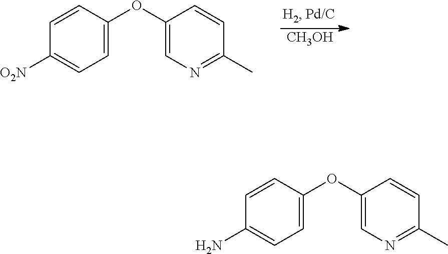

To a solution of 2-methyl-5-(4-nitrophenoxy)pyridine (1.5 g, 6.51 mmol) in CH₃OH (10.0 mL) was added Pd/C (693.4 mg, dry) at room temperature. The resulting mixture was stirred at room temperature for 2 h under H₂. After the reaction was completed, the resulting mixture was filtered. The filtrate was concentrated under reduced pressure. The residue was purified by flash column chromatography with CH₂Cl₂/CH₃OH (83/17, v/v) to afford 4-[(6-methylpyridin-3-yl)oxy]aniline (1.0 g, 76%) as a yellow solid. LCMS (ESI, m/z): [M+H]⁺=201.0.

### Step 3: Synthesis of N-[4-[(6-methylpyridin-3-yl)oxy]phenyl]-6-nitroquinazolin-4-amine

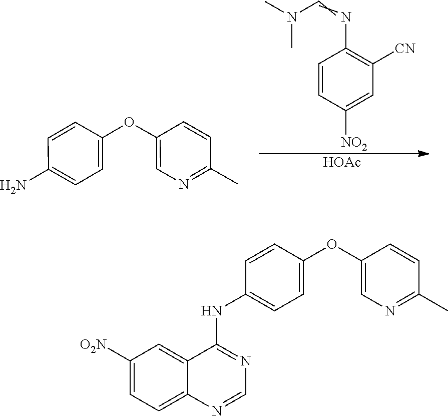 [SMILES: Cc1ccc(Oc2ccc(N)cc2)cn1 ; CN(C)C=Nc1ccc([N+](=O)[O-])cc1C#N ; Cc1ccc(Oc2ccc(Nc3ncnc4ccc([N+](=O)[O-])cc34)cc2)cn1 ; "HOAc"]

To a solution of 4-[(6-methylpyridin-3-yl)oxy]aniline (1.0 g, 4.99 mmol) in AcOH (10.0 mL) was added N′-(2-cyano-4-nitrophenyl)-N,N-dimethylformimidamide (1.1 g, 4.99 mmol) at room temperature. The resulting mixture was stirred at 85° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (4/1, v/v) to afford N-[4-[(6-methylpyridin-3-yl)oxy]phenyl]-6-nitroquinazolin-4-amine (1.5 g, 80%) as a yellow solid. LCMS (ESI, m/z): [M+H]⁺=374.1.

### Step 4: Synthesis of N4-[4-[(6-methylpyridin-3-yl)oxy]phenyl]quinazoline-4,6-diamine

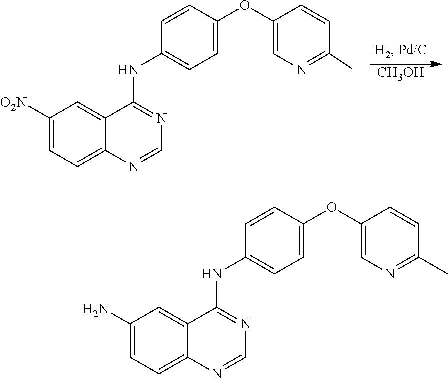

To a solution of N-[4-[(6-methylpyridin-3-yl)oxy]phenyl]-6-nitroquinazolin-4-amine (1.5 g, 4.02 mmol) in CH₃OH (10.0 mL) was added Pd/C (427.5 mg, dry) at room temperature. The resulting mixture was stirred at room temperature for 2 h under H₂. After the reaction was completed, the resulting mixture was filtered. The filtrate was concentrated under reduced pressure. The residue was purified by reverse phase flash column chromatography with acetonitrile/water (74/26, v/v) to afford N4-[4-[(6-methylpyridin-3-yl)oxy]phenyl]quinazoline-4,6-diamine (1.0 g, 72%) as a yellow solid. LCMS (ESI, m/z): [M+H]⁺=344.1.

### Step 5: Synthesis of N-[4-([4-[(6-methylpyridin-3-yl)oxy]phenyl]amino)quinazolin-6-yl]but-2-ynamide (Compound 9)

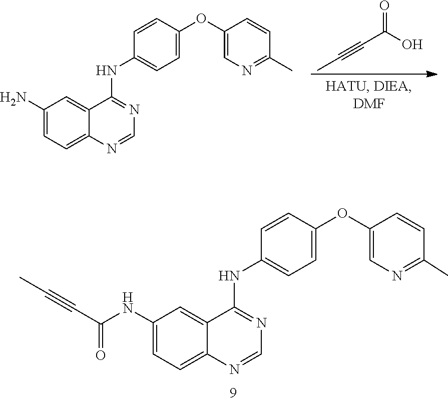

To a solution of N4-[4-[(6-methylpyridin-3-yl)oxy]phenyl]quinazoline-4,6-diamine (300.0 mg, 0.87 mmol) in DMF (5.0 mL) was added 2-butynoic acid (110.2 mg, 1.31 mmol), DIEA (338.7 mg, 2.62 mmol) and HATU (498.3 mg, 1.31 mmol) at room temperature. The resulting mixture was stirred at room temperature for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with acetonitrile/water (3/7, v/v) and then purified by Prep-HPLC with the following conditions (Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 34% B to 45% B in 8 min; 254 nm) to afford N-[4-([4-[(6-methylpyridin-3-yl)oxy]phenyl]amino)quinazolin-6-yl]but-2-ynamide (Compound 9) (24.4 mg, 6%) as a yellow solid. LCMS (ESI, m/z): [M+H]⁺=410.2. ¹H NMR (300 MHz, DMSO-d₆): δ 10.92 (s, 1H), 9.85 (s, 1H), 8.73 (s, 1H), 8.50 (s, 1H), 8.27 (d, J=2.7 Hz, 1H), 7.81-7.77 (m, 4H), 7.39-7.36 (m, 1H), 7.28 (d, J=8.4 Hz, 1H), 7.08 (d, J=9.0 Hz, 2H), 2.47 (s, 3H), 2.09 (s, 3H).

### Example S10. Synthesis of N-[4-([4-[(5-methylpyrazin-2-yl)oxy]phenyl]amino)quinazolin-6-yl]but-2-ynamide (Compound 10)

### Step 1: Synthesis of 2-methyl-5-(4-nitrophenoxy)pyrazine

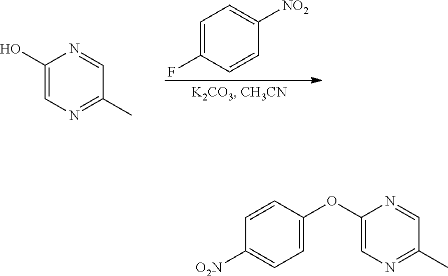

To a solution of 5-methylpyrazin-2-ol (6.3 g, 56.94 mmol) in CH₃CN (100.0 mL) was added K₂CO₃ (8.8 g, 63.31 mmol) and 4-fluoronitrobenzene (7.7 g, 54.713 mmol). The mixture was stirred at 80° C. for 16 h. After the reaction was completed, the reaction mixture was filtered. The filtrate was concentrated under reduced pressure. The residue was purified by flash column chromatography with petroleum ether/ethyl acetate (7/3, v/v) to afford 2-methyl-5-(4-nitrophenoxy)pyrazine (2.8 g, 21%) as a light yellow solid. LCMS (ESI, m/z): [M+H]⁺=232.0.

### Step 2: Synthesis of 4-[(5-methylpyrazin-2-yl)oxy]aniline

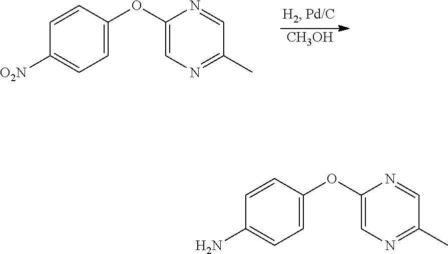

To a solution of 2-methyl-5-(4-nitrophenoxy)pyrazine (2.8 g, 12.02 mmol) in CH₃OH (30.0 mL) was added Pd/C (420.0 mg, dry) at room temperature. The resulting mixture was stirred at room temperature for 2 h under H₂. After the reaction was completed, the resulting mixture was filtered. The filtrate was concentrated under reduced pressure. The residue was purified by flash column chromatography with petroleum ether/ethyl acetate (8/2, v/v) to afford 4-[(5-methylpyrazin-2-yl)oxy]aniline (900.0 mg, 37%) as a light yellow solid. LCMS (ESI, m/z): [M+H]⁺=202.1.

### Step 3: Synthesis of N-[4-[(5-methylpyrazin-2-yl)oxy]phenyl]-6-nitroquinazolin-4-amine

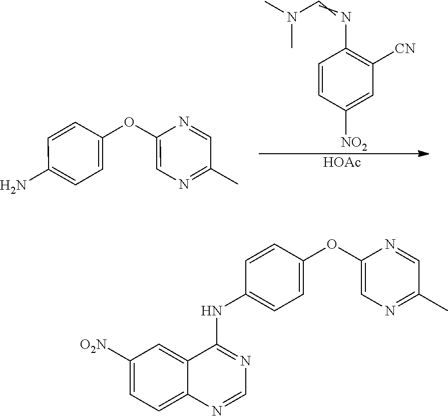 [SMILES: Cc1cnc(Oc2ccc(N)cc2)cn1 ; CN(C)C=Nc1ccc([N+](=O)[O-])cc1C#N ; Cc1cnc(Oc2ccc(Nc3ncnc4ccc([N+](=O)[O-])cc34)cc2)cn1 ; "HOAc"]

To a solution of 4-[(5-methylpyrazin-2-yl)oxy]aniline (900.0 mg, 4.47 mmol) in AcOH (10.0 mL) was added N′-(2-cyano-4-nitrophenyl)-N,N-dimethylformimidamide (975.9 mg, 4.47 mmol) at room temperature. The resulting mixture was stirred at 85° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/9, v/v) to afford N-[4-[(5-methylpyrazin-2-yl)oxy]phenyl]-6-nitroquinazolin-4-amine (1.5 g, 89%) as a yellow solid. LCMS (ESI, m/z): [M+H]⁺=375.1.

### Step 4: Synthesis of N4-[4-[(5-methylpyrazin-2-yl)oxy]phenyl]quinazoline-4,6-diamine

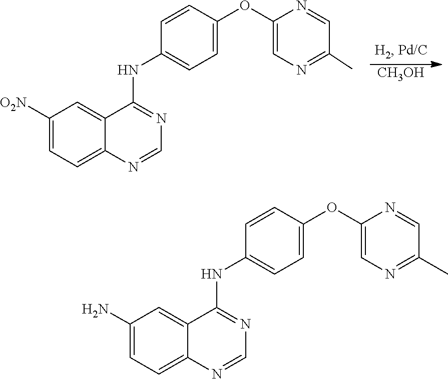

To a solution of N-[4-[(5-methylpyrazin-2-yl)oxy]phenyl]-6-nitroquinazolin-4-amine (500.0 mg, 1.34 mmol) in CH₃OH (10.0 mL) was added Pd/C (142.1 mg, dry) at room temperature. The resulting mixture was stirred at room temperature for 2 h under H₂. After the reaction was completed, the resulting mixture was filtered. The filtrate was concentrated under reduced pressure. The residue was purified by reverse phase flash column chromatography with acetonitrile/water (26/74, v/v) to afford N4-[4-[(5-methylpyrazin-2-yl)oxy]phenyl]quinazoline-4,6-diamine (400.0 mg, 86%) as a yellow solid. LCMS (ESI, m/z): [M+H]⁺=345.1.

### Step 5: Synthesis of N-[4-([4-[(5-methylpyrazin-2-yl)oxy]phenyl]amino)quinazolin-6-yl]but-2-ynamide (Compound 10)

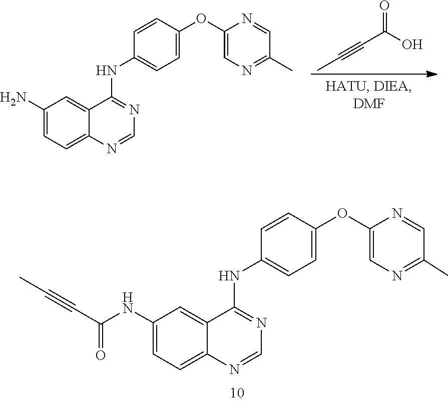

To a solution of N4-[4-[(5-methylpyrazin-2-yl)oxy]phenyl]quinazoline-4,6-diamine (500.0 mg, 1.45 mmol) in DMF (5.0 mL) was added 2-butynoic acid (183.1 mg, 2.18 mmol), DIEA (562.9 mg, 4.36 mmol) and HATU (828.1 mg, 2.18 mmol) at room temperature. The resulting mixture was stirred at room temperature for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with acetonitrile/water (64/36, v/v) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 26% B to 56% B in 10 min; 254 nm) to afford N-[4-([4-[(5-methylpyrazin-2-yl)oxy]phenyl]amino)quinazolin-6-yl]but-2-ynamide (Compound 10) (39.6 mg, 6%) as a yellow solid. LCMS (ESI, m/z): [M+H]⁺=411.2. 1H NMR (400 MHz, CD₃OD): δ 8.66 (d, J=2.0 Hz, 1H), 8.49 (s, 1H), 8.32 (d, J=1.2 Hz, 1H), 8.08 (s, 1H), 7.84-7.76 (m, 4H), 7.25-7.21 (m, 2H), 2.52 (s, 3H), 2.10 (s, 3H).

### Example S11. Synthesis of 4-hydroxy-N-[4-[(4-[[1,2,4]triazolo[1,5-a]pyridin-7-yloxy]phenyl)amino]quinazolin-6-yl]but-2-ynamide (Compound 11)

### Step 1: Synthesis of methyl 4-(oxan-2-yloxy)but-2-ynoate

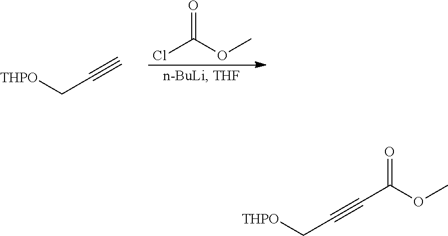

To a solution of 2-(prop-2-yn-1-yloxy)oxane (6.0 g, 43.37 mmol) in THF (80.0 mL) was added n-BuLi in hexanes (18.0 mL, 2.5 mol/L) at −70° C. under N₂. The mixture was stirred at −70° C. for 1 h. Then methyl chloroformate (6.30 g, 66.67 mmol) was added dropwise to the mixture at −70° C. The mixture was stirred at −70° C. for another 2 h. After the reaction was completed, the reaction mixture was quenched with NH₄Cl and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (82/18, v/v) to afford methyl 4-(oxan-2-yloxy)but-2-ynoate (7.4 g, 86%) as a colorless oil.

### Step 2: Synthesis of 4-((tetrahydro-2H-pyran-2-yl)oxy)but-2-ynoic acid

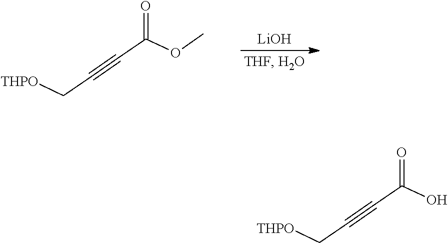

To a solution of methyl 4-(oxan-2-yloxy)but-2-ynoate (2.2 g, 11.09 mmol) in THF (30.0 mL) was added a solution of LiOH (957.0 mg, 39.96 mmol) in H₂O (20.0 mL). The mixture was stirred at room temperature for 16 h. After the reaction was completed, the pH value of the mixture was adjusted to pH 5 with HCl (1 mol/L) and then extracted with Et₂O. The combined organic layer was washed with brine, dried over anhydrous sodium sulfate and filtered. The filtrate was concentrated under vacuum to afford 4-((tetrahydro-2H-pyran-2-yl)oxy)but-2-ynoic acid (2.0 g, crude) as a colorless oil. LCMS (ESI, m/z): [M−H]⁺=183.1.

### Step 3: Synthesis of 4-(oxan-2-yloxy)-N-[4-[(4-[[1,2,4]triazolo[1,5-a]pyridin-7-yloxy]phenyl)amino]quinazolin-6-yl]but-2-ynamide

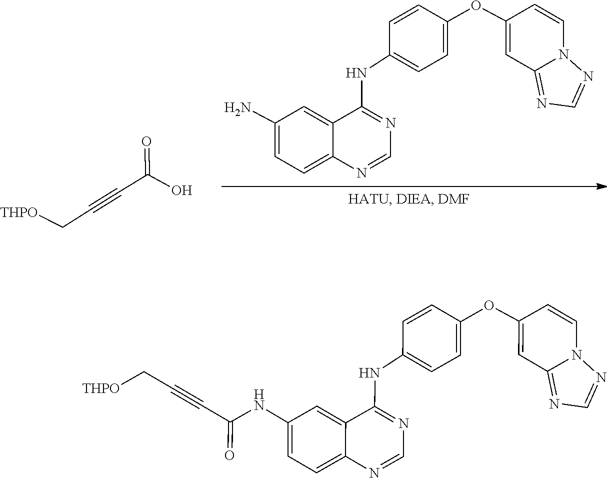

To a solution of N4-(4-[[1,2,4]triazolo[1,5-a]pyridin-7-yloxy]phenyl)quinazoline-4,6-diamine (300.0 mg, 0.81 mmol) in DMF (10.0 mL) was added 4-((tetrahydro-2H-pyran-2-yl)oxy)but-2-ynoic acid (320.0 mg, 1.73 mmol), DIEA (625.0 mg, 4.83 mmol) and HATU (711.4 mg, 1.87 mmol). The mixture was stirred at room temperature for 2 h. After the reaction was completed, the resulting mixture was diluted with H₂O and extracted with CH₂Cl₂. The combined organic layer was washed with brine, dried over anhydrous sodium sulfate and filtered. The filtrate was concentrated under vacuum. The residue was purified by flash column chromatography with CH₂Cl₂/CH₃OH (90/10, v/v) to afford 4-(oxan-2-yloxy)-N-[4-[(4-[[1,2,4]triazolo[1,5-a]pyridin-7-yloxy]phenyl)amino]quinazolin-6-yl]but-2-ynamide (160.0 mg, 36%) as a yellow solid. LCMS (ESI, m/z): [M+H]⁺=536.2.

### Step 4: Synthesis of 4-hydroxy-N-[4-[(4-[[1,2,4]triazolo[1,5-a]pyridin-7-yloxy]phenyl)amino]quinazolin-6-yl]but-2-ynamide (Compound 11)

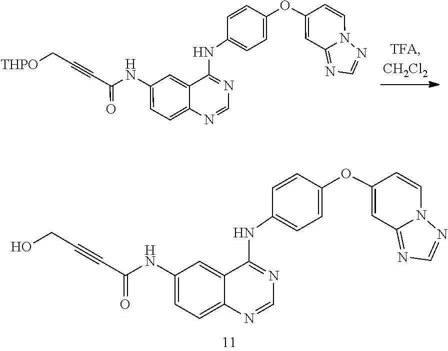

To a solution of 4-(oxan-2-yloxy)-N-[4-[(4-[[1,2,4]triazolo[1,5-a]pyridin-7-yloxy]phenyl)amino]quinazolin-6-yl]but-2-ynamide (140.0 mg, 0.26 mmol) in CH₂Cl₂ (4.0 mL) was added TFA (2.0 mL) at room temperature. The resulting mixture was stirred at room temperature for 2 h. After the reaction was completed, the pH value of the mixture was adjusted to pH 7 with NaHCO₃(aq.) and then extracted with ethyl acetate. The combined organic layer was washed with brine, dried over anhydrous sodium sulfate and filtered. The filtrate was concentrated under vacuum. The residue was purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 8% B to 22% B in 7 min; 254 nm) to afford 4-hydroxy-N-[4-[(4-[[1,2,4]triazolo[1,5-a]pyridin-7-yloxy]phenyl)amino]quinazolin-6-yl]but-2-ynamide (Compound 11) (18.6 mg, 15%) as a white solid. LCMS (ESI, m/z): [M+H]⁺=452.2. ¹H NMR (400 MHz, DMSO-d₆): δ 11.08 (s, 1H), 10.05 (s, 1H), 8.95-8.93 (m, 1H), 8.78 (s, 1H), 8.60-8.54 (m, 1H), 8.40 (s, 1H), 7.91-7.77 (m, 4H), 7.31-7.26 (m, 2H), 7.05-7.02 (m, 2H), 5.58 (s, 1H), 4.33 (d, J=3.6 Hz, 2H).

### Example S12. Synthesis of 5-hydroxy-N-[4-[(4-[[1,2,4]triazolo[1,5-a]pyridin-7-yloxy]phenyl)amino]quinazolin-6-yl]pent-2-ynamide (Compound 12)

### Step 1: Synthesis of methyl 5-[(tert-butyldimethylsilyl)oxy]pent-2-ynoate

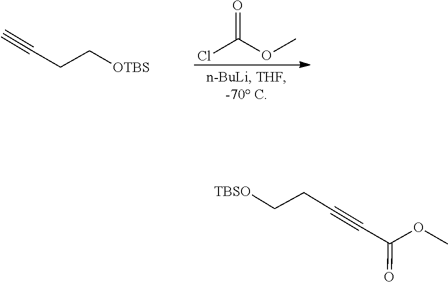

To a solution of (but-3-yn-1-yloxy)(tert-butyl)dimethylsilane (3.1 g, 16.71 mmol) in THF (50.0 mL) was added n-BuLi in hexanes (8.0 mL, 2.5 mol/L) at −70° C. under N₂. The mixture was stirred at −70° C. for 1 h. Then methyl chloroformate (2.6 g, 27.52 mmol) was added dropwise to the mixture at −70° C. The mixture was stirred at −70° C. for another 2 h. After the reaction was completed, the mixture was quenched with NH₄Cl solution. The mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/EtOAc (95/5, v/v) to afford methyl 5-[(tert-butyldimethylsilyl)oxy]pent-2-ynoate (2.1 g, 52%) as a colorless oil.

### Step 2: Synthesis of 5-[(tert-butyldimethylsilyl)oxy]pent-2-ynoic acid

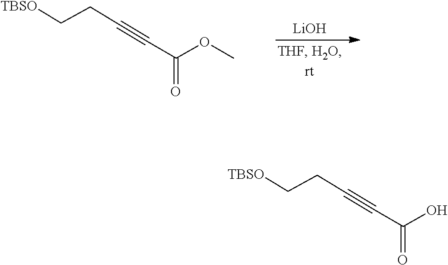 [SMILES: COC(=O)C#CCCO[Si](C)(C)C(C)(C)C ; "LiOH" ; CC(C)(C)[Si](C)(C)OCCC#CC(=O)O]

To a solution of methyl 5-[(tert-butyldimethylsilyl)oxy]pent-2-ynoate (2.1 g, 8.71 mmol) in THF (20.0 mL) was added a solution of LiOH (950.0 mg, 39.67 mmol) in H₂O (15.0 mL) at room temperature. The mixture was stirred at room temperature for 16 h. After the reaction was completed, the pH value of the mixture was adjusted to pH 5 with HCl (1 mol/L) and then extracted with Et₂O. The combined organic layer was washed with brine, dried over anhydrous sodium sulfate and filtered. The filtrate was concentrated under vacuum to afford 5-[(tert-butyldimethylsilyl)oxy]pent-2-ynoic acid (1.0 g, crude) as a light yellow oil. LCMS (ESI, m/z): [M−H]⁺=227.1.

### Step 3: Synthesis of 5-[(tert-butyldimethylsilyl)oxy]-N-[4-[(4-[[1,2,4]triazolo[1,5-a]pyridin-7-yloxy]phenyl)amino]quinazolin-6-yl]pent-2-ynamide

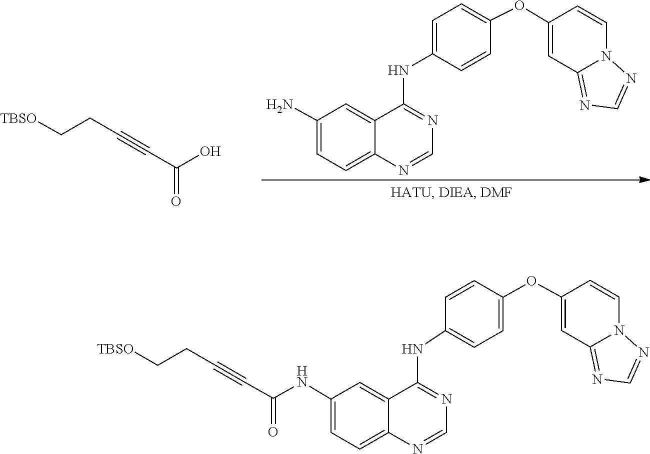 [SMILES: CC(C)(C)[Si](C)(C)OCCC#CC(=O)O ; Nc1ccc2ncnc(Nc3ccc(Oc4ccn5ncnc5c4)cc3)c2c1 ; CC(C)(C)[Si](C)(C)OCCC#CC(=O)Nc1ccc2ncnc(Nc3ccc(Oc4ccn5ncnc5c4)cc3)c2c1]

To a solution of 5-[(tert-butyldimethylsilyl)oxy]pent-2-ynoic acid (300.0 mg, 1.31 mmol) in DMF (8.0 mL) was added N4-(4-[[1,2,4]triazolo[1,5-a]pyridin-7-yloxy]phenyl)quinazoline-4,6-diamine (485.3 mg, 1.31 mmol), DIEA (1697.9 mg, 13.14 mmol) and HATU (1498.5 mg, 3.94 mmol) at room temperature. The mixture was stirred at room temperature for 1 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford 5-[(tert-butyldimethylsilyl)oxy]-N-[4-[(4-[[1,2,4]triazolo[1,5-a]pyridin-7-yloxy]phenyl)amino]quinazolin-6-yl]pent-2-ynamide (400.0 mg, 52%) as a yellow solid. LCMS (ESI, m/z): [M+H]⁺=580.2.

### Step 4: Synthesis of 5-hydroxy-N-[4-[(4-[[1,2,4]triazolo[1,5-a]pyridin-7-yloxy]phenyl)amino]quinazolin-6-yl]pent-2-ynamide (Compound 12)

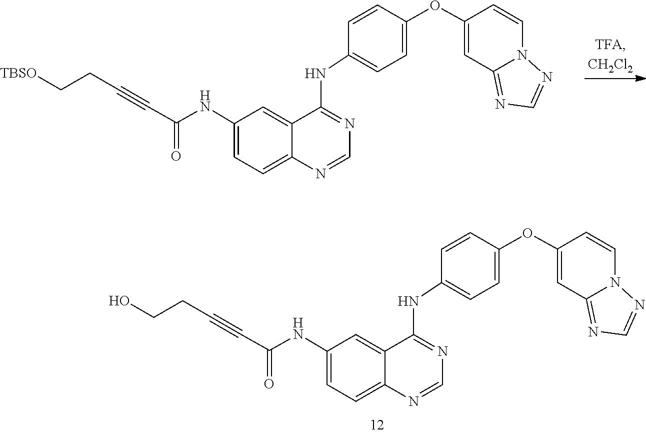 [SMILES: CC(C)(C)[Si](C)(C)OCCC#CC(=O)Nc1ccc2ncnc(Nc3ccc(Oc4ccn5ncnc5c4)cc3)c2c1 ; O=C(C#CCCO)Nc1ccc2ncnc(Nc3ccc(Oc4ccn5ncnc5c4)cc3)c2c1]

A mixture of 5-[(tert-butyldimethylsilyl)oxy]-N-[4-[(4-[[1,2,4]triazolo[1,5-a]pyridin-7-yloxy]phenyl)amino]quinazolin-6-yl]pent-2-ynamide (350.0 mg, 0.60 mmol) and TFA (5.0 mL) in CH₂Cl₂ (5.0 mL) was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (90/10, v/v) and then purified by Prep-HPLC with the following conditions (Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 26% B to 35% B in 8 min; 254/220 nm) to afford 5-hydroxy-N-[4-[(4-[[1,2,4]triazolo[1,5-a]pyridin-7-yloxy]phenyl)amino]quinazolin-6-yl]pent-2-ynamide (Compound 12) (36.9 mg, 13%) as a white solid. LCMS (ESI, m/z): [M+H]⁺=466.2. ¹H NMR (400 MHz, DMSO-d₆): δ 10.93 (s, 1H), 9.93 (s, 1H), 8.95 (d, J=7.2 Hz, 1H), 8.75 (s, 1H), 8.55 (s, 1H), 8.41 (s, 1H), 7.93 (d, J=8.8 Hz, 2H), 7.84-7.77 (m, 2H), 7.28 (d, J=8.8 Hz, 2H), 7.06-7.03 (m, 2H), 5.03-5.01 (m, 1H), 3.66-3.61 (m, 2H), 2.62-2.58 (m, 2H).

### Example S13. Synthesis of N-[4-([4-[(1-methyl-2-oxopyridin-4-yl)methyl]phenyl]amino)quinazolin-6-yl]but-2-ynamide (Compound 13)

### Step 1: Synthesis of 1-methyl-4-[(4-nitrophenyl)methyl]pyridin-2-one

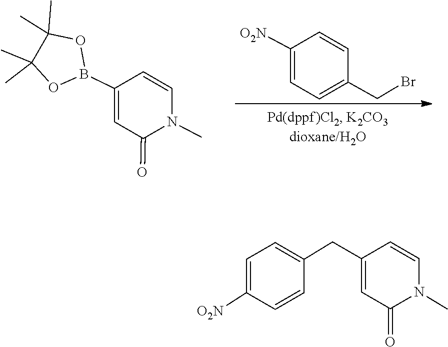

To a solution of 1-(bromomethyl)-4-nitrobenzene (800.0 mg, 3.70 mmol) in 1,4-dioxane/H₂O (5.0/1.0 mL) was added 1-methyl-4-(4,4,5,5-tetramethyl-1,3,2-dioxaborolan-2-yl)pyridin-2-one (1.04 g, 4.44 mmol), K₂CO₃ (1.53 g, 11.11 mmol) and Pd(dppf)Cl₂ (271.0 mg, 0.37 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (4/1, v/v) to afford 1-methyl-4-[(4-nitrophenyl)methyl]pyridin-2-one (400.0 mg, 44%) as a yellow solid. LCMS (ESI, m/z): [M+H]⁺=245.1

### Step 2: Synthesis of 4-[(4-aminophenyl)methyl]-1-methylpyridin-2-one

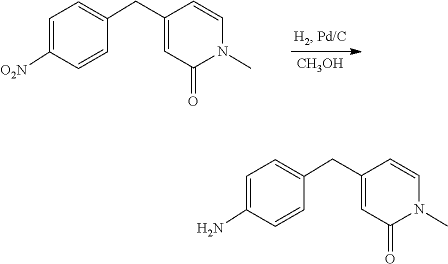

To a solution of 1-methyl-4-[(4-nitrophenyl)methyl]pyridin-2-one (500.0 mg, 2.04 mmol) in CH₃OH (10.0 mL) was added Pd/C (217.8 mg, dry) at room temperature. The resulting mixture was stirred at room temperature for 2 h under H₂. After the reaction was completed, the resulting mixture was filtered. The filtrate was concentrated under reduced pressure. The residue was purified by flash column chromatography with CH₂Cl₂/CH₃OH (93/7, v/v) to afford 4-[(4-aminophenyl)methyl]-1-methylpyridin-2-one (400.0 mg, 91%) as a yellow solid. LCMS (ESI, m/z): [M+H]⁺=215.1.

### Step 3: Synthesis of 1-methyl-4-([4-[(6-nitroquinazolin-4-yl)amino]phenyl]methyl)pyridin-2-one

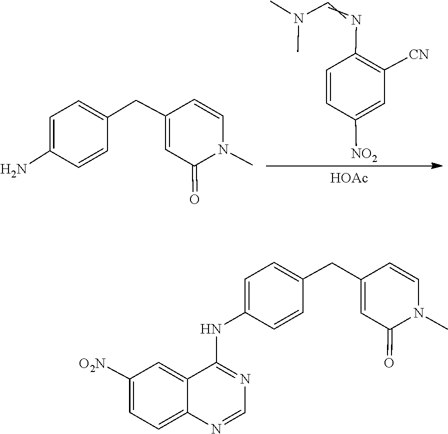 [SMILES: Cn1ccc(Cc2ccc(N)cc2)cc1=O ; CN(C)C=Nc1ccc([N+](=O)[O-])cc1C#N ; Cn1ccc(Cc2ccc(Nc3ncnc4ccc([N+](=O)[O-])cc34)cc2)cc1=O ; "HOAc"]

To a solution of 4-[(4-aminophenyl)methyl]-1-methylpyridin-2-one (400.0 mg, 1.87 mmol) in AcOH (10.0 mL) was added N′-(2-cyano-4-nitrophenyl)-N,N-dimethylformimidamide (407.4 mg, 1.87 mmol) at room temperature. The resulting mixture was stirred at 85° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (85/15, v/v) to afford 1-methyl-4-([4-[(6-nitroquinazolin-4-yl)amino]phenyl]methyl)pyridin-2-one (500.0 mg, 69%) as a yellow solid. LCMS (ESI, m/z): [M+H]⁺=388.1.

### Step 4: Synthesis of 4-([4-[(6-aminoquinazolin-4-yl)amino]phenyl]methyl)-1-methylpyridin-2-one

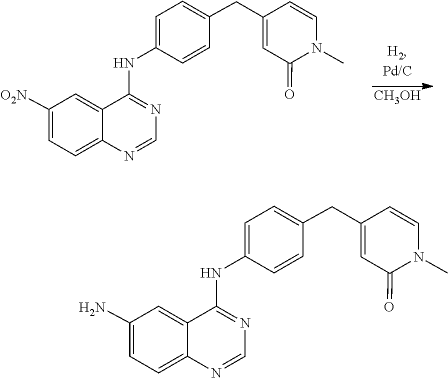

To a solution of 1-methyl-4-([4-[(6-nitroquinazolin-4-yl)amino]phenyl]methyl)pyridin-2-one (500.0 mg, 1.29 mmol) in CH₃OH (10.0 mL) was added Pd/C (137.4 mg, dry) at room temperature. The resulting mixture was stirred at room temperature for 2 h under H₂. After the reaction was completed, the resulting mixture was filtered. The filtrate was concentrated under reduced pressure. The residue was purified by flash column chromatography with CH₂Cl₂/CH₃OH (9/1, v/v) to afford 4-([4-[(6-aminoquinazolin-4-yl)amino]phenyl]methyl)-1-methylpyridin-2-one (400.0 mg, 86%) as a yellow solid. LCMS (ESI, m/z): [M+H]⁺=358.2.

### Step 5: Synthesis of N-[4-([4-[(1-methyl-2-oxopyridin-4-yl)methyl]phenyl]amino)quinazolin-6-yl]but-2-ynamide (Compound 13)

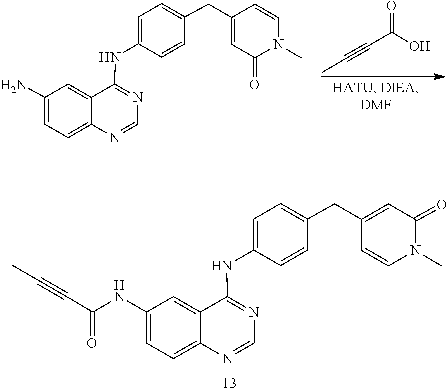

To a solution of 4-([4-[(6-aminoquinazolin-4-yl)amino]phenyl]methyl)-1-methylpyridin-2-one (200.0 mg, 0.56 mmol) in DMF (5.0 mL) was added 2-butynoic acid (61.2 mg, 0.73 mmol), DIEA (723.2 mg, 5.60 mmol) and HATU (340.4 mg, 0.89 mmol) at room temperature. The resulting mixture was stirred at room temperature for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with acetonitrile/water (64/36, v/v) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 12% B to 42% B in 10 min; 254 nm) to afford N-[4-([4-[(1-methyl-2-oxopyridin-4-yl)methyl]phenyl]amino)quinazolin-6-yl]but-2-ynamide (Compound 13) (1.1 mg, 0.5%) as a white solid. LCMS (ESI, m/z): [M+H]⁺=424.2. ¹H NMR (300 MHz, DMSO-d₆): δ 10.90 (s, 1H), 9.78 (s, 1H), 8.71 (s, 1H), 8.49 (s, 1H), 7.78-7.70 (m, 4H), 7.58 (d, J=6.9 Hz, 1H), 7.26 (d, J=8.7 Hz, 2H), 6.25 (s, 1H), 6.10-6.08 (m, 1H), 3.75 (s, 2H), 3.37 (s, 3H), 2.09 (s, 3H).

### Example S14. Synthesis of N-[4-[(4-[[1,2,4]triazolo[1,5-a]pyridin-7-ylmethyl]phenyl)amino]quinazolin-6-yl]but-2-ynamide (Compound 14)

### Step 1: Synthesis of 7-(4,4,5,5-tetramethyl-1,3,2-dioxaborolan-2-yl)-[1,2,4]triazolo[1,5-a]pyridine

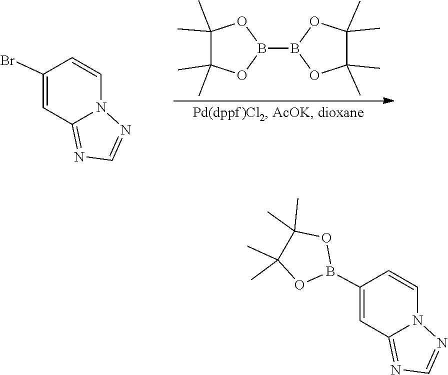

To a solution of 7-bromo-[1,2,4]triazolo[1,5-a]pyridine (1.0 g, 5.05 mmol) in 1,4-dioxane (10.0 mL) was added bis(pinacolato)diboron (3.8 g, 15.15 mmol), AcOK (1.5 g, 15.15 mmol) and Pd(dppf)Cl₂ (369.5 mg, 0.51 mmol) at room temperature under N₂. The resulting mixture was stirred at 80° C. for 16 h under N₂. After the reaction was completed, the resulting mixture was diluted with H₂O and extracted with ethyl acetate. The aqueous layer was concentrated under reduced pressure to afford 7-(4,4,5,5-tetramethyl-1,3,2-dioxaborolan-2-yl)-[1,2,4]triazolo[1,5-a]pyridine (1.0 g, crude) as a white solid. LCMS (ESI, m/z): [M+H]⁺=246.1

### Step 2: Synthesis of 7-[(4-nitrophenyl)methyl]-[1,2,4]triazolo[1,5-a]pyridine

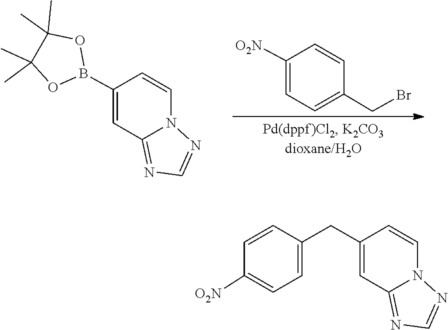 [SMILES: CC1(C)OB(c2ccn3ncnc3c2)OC1(C)C ; O=[N+]([O-])c1ccc(CBr)cc1 ; O=[N+]([O-])c1ccc(Cc2ccn3ncnc3c2)cc1]

To a solution of 1-(bromomethyl)-4-nitrobenzene (1.0 g, 4.63 mmol) in 1,4-dioxane/H₂O (10.0/2.0 mL) was added K₂CO₃ (1.9 g, 13.89 mmol), 7-(4,4,5,5-tetramethyl-1,3,2-dioxaborolan-2-yl)-[1,2,4]triazolo[1,5-a]pyridine (3.4 g, 13.89 mmol) and Pd(dppf)Cl₂ (338.7 mg, 0.46 mmol) at room temperature under N₂. The resulting mixture was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (47/53, v/v) to afford 7-[(4-nitrophenyl)methyl]-[1,2,4]triazolo[1,5-a]pyridine (500.0 mg, 42%) as a white solid. LCMS (ESI, m/z): [M+H]⁺=255.0.

### Step 3: Synthesis of 4-[[1,2,4]triazolo[1,5-a]pyridin-7-ylmethyl]aniline

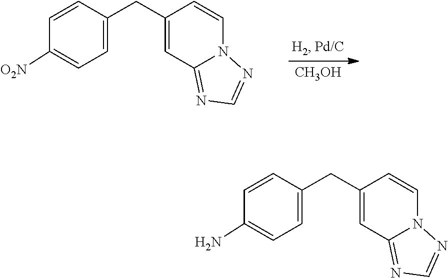

To a solution of 7-[(4-nitrophenyl)methyl]-[1,2,4]triazolo[1,5-a]pyridine (450.0 mg, 1.77 mmol) in MeOH (10.0 mL) was added Pd/C (188.4 mg, dry) at room temperature under N₂. The resulting mixture was stirred at room temperature for 2 h under H₂. After the reaction was completed, the resulting mixture was filtered. The filtrate was concentrated under reduced pressure. The residue was purified by flash column chromatography with CH₂Cl₂/CH₃OH (9/1, v/v) to afford 4-[[1,2,4]triazolo[1,5-a]pyridin-7-ylmethyl]aniline (300.0 mg, 75%) as a yellow solid. LCMS (ESI, m/z): [M+H]⁺=225.1.

### Step 4: Synthesis of 6-nitro-N-(4-[[1,2,4]triazolo[1,5-a]pyridin-7-ylmethyl]phenyl)quinazolin-4-amine

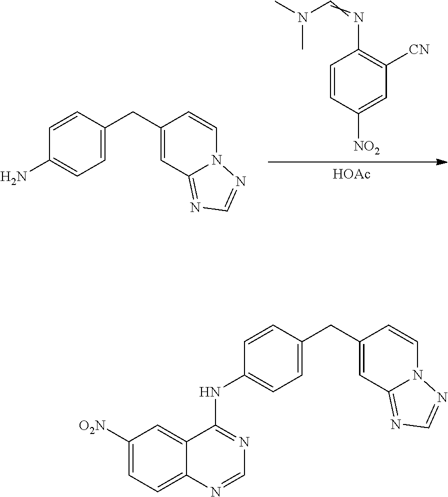 [SMILES: Nc1ccc(Cc2ccn3ncnc3c2)cc1 ; CN(C)C=Nc1ccc([N+](=O)[O-])cc1C#N ; O=[N+]([O-])c1ccc2ncnc(Nc3ccc(Cc4ccn5ncnc5c4)cc3)c2c1 ; "HOAc"]

To a solution of 4-[[1,2,4]triazolo[1,5-a]pyridin-7-ylmethyl]aniline (250.0 mg, 1.11 mmol) in HOAc (10.0 mL) was added N′-(2-cyano-4-nitrophenyl)-N,N-dimethylformimidamide (243.3 mg, 1.11 mmol) at room temperature. The resulting mixture was stirred at 85° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (83/17, v/v) to afford 6-nitro-N-(4-[[1,2,4]triazolo[1,5-a]pyridin-7-ylmethyl]phenyl)quinazolin-4-amine (200.0 mg, 45%) as a yellow solid. LCMS (ESI, m/z): [M+H]⁺=398.1

### Step 5: Synthesis of N4-(4-[[1,2,4]triazolo[1,5-a]pyridin-7-ylmethyl]phenyl)quinazoline-4,6-diamine

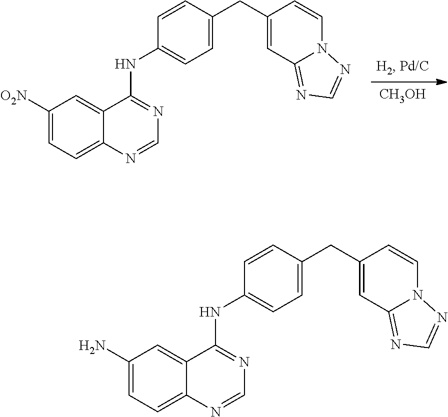 [SMILES: O=[N+]([O-])c1ccc2ncnc(Nc3ccc(Cc4ccn5ncnc5c4)cc3)c2c1 ; "CH3OH" ; "H2, Pd/C" ; Nc1ccc2ncnc(Nc3ccc(Cc4ccn5ncnc5c4)cc3)c2c1]

To a solution of 6-nitro-N-(4-[[1,2,4]triazolo[1,5-a]pyridin-7-ylmethyl]phenyl)quinazolin-4-amine (150.0 mg, 0.38 mmol) in CH₃OH (10.0 mL) was added Pd/C (40.1 mg, dry) at room temperature under N₂. The resulting mixture was stirred at room temperature for 2 h under H₂. After the reaction was completed, the resulting mixture was filtered. The filtrate was concentrated under reduced pressure. The residue was purified by flash column chromatography with CH₂Cl₂/CH₃OH (88/12, v/v) to afford N4-(4-[[1,2,4]triazolo[1,5-a]pyridin-7-ylmethyl]phenyl)quinazoline-4,6-diamine (100.0 mg, 72%) as a yellow solid. LCMS (ESI, m/z): [M+H]⁺=368.1.

### Step 6: Synthesis of N-[4-[(4-[[1,2,4]triazolo[1,5-a]pyridin-7-ylmethyl]phenyl)amino]quinazolin-6-yl]but-2-ynamide (Compound 14)

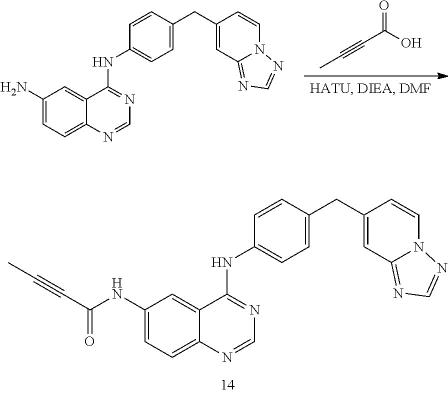

To a solution of N4-(4-[[1,2,4]triazolo[1,5-a]pyridin-7-ylmethyl]phenyl)quinazoline-4,6-diamine (400.0 mg, 1.09 mmol) in DMF (5.0 mL) was added 2-butynoic acid (119.0 mg, 1.42 mmol), DIEA (1.4 g, 10.89 mmol) and HATU (662.3 mg, 1.74 mmol) at room temperature. The resulting mixture was stirred at room temperature for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse flash column chromatography with acetonitrile/water (54/46, v/v) and then purified by Prep-HPLC with the following conditions (Column:) (Bridge Prep OBD C18 Column, 30×150 mm, 5 um; Mobile Phase A: Water (10 mmol/L NH₄HCO₃), Mobile Phase B: ACN; Flow rate: 60 mL/min; Gradient: 23% B to 53% B in 10 min; 254 nm) to afford N-[4-[(4-[[1,2,4]triazolo[1,5-a]pyridin-7-ylmethyl]phenyl)amino]quinazolin-6-yl]but-2-ynamide (Compound 14) (5.6 mg, 1%) as a white solid. LCMS (ESI, m/z): [M+H]⁺=434.2. ¹H NMR (300 MHz, DMSO-d₆): δ 10.91 (s, 1H), 9.80 (s, 1H), 8.86 (d, J=6.9 Hz, 1H), 8.71 (s, 1H). 8.48-8.44 (m, 2H), 7.78-7.72 (m, 5H) 7.33 (d, J=8.4 Hz, 2H), 7.10-7.08 (m, 1H), 4.11 (s, 2H), 2.09 (s, 3H).

### Example S15. Synthesis of N-[4-([4-[(1-ethyl-2-oxopyridin-4-yl)oxy]-3-methylphenyl]amino)quinazolin-6-yl]but-2-ynamide (Compound 15)

### Step 1: Synthesis of 1-ethyl-4-(2-methyl-4-nitrophenoxy)pyridin-2-one

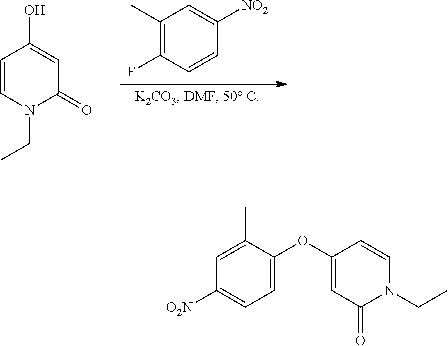

A mixture of 1-ethyl-4-hydroxypyridin-2-one (2.0 g, 14.37 mmol), 1-fluoro-2-methyl-4-nitrobenzene (2.7 g, 17.25 mmol) and K₂CO₃ (4.0 g, 28.75 mmol) in DMF (30.0 mL) was stirred at 50° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (91/9, v/v) to afford 1-ethyl-4-(2-methyl-4-nitrophenoxy)pyridin-2-one (3.0 g, 76%) as a yellow solid. LCMS (ESI, m/z): [M+H]⁺=275.1.

### Step 2: Synthesis of 4-(4-amino-2-methylphenoxy)-1-ethylpyridin-2-one

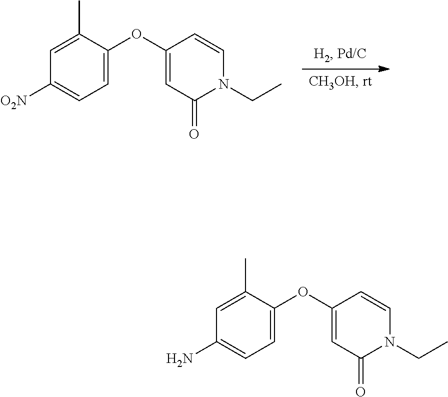 [SMILES: CCn1ccc(Oc2ccc([N+](=O)[O-])cc2C)cc1=O ; "CH3OH, rt" ; "H2, Pd/C" ; CCn1ccc(Oc2ccc(N)cc2C)cc1=O]

To a solution of 1-ethyl-4-(2-methyl-4-nitrophenoxy)pyridin-2-one (2.9 g, 10.57 mmol) in CH₃OH (30.0 mL) was added Pd/C (0.9 g, dry) at room temperature. The mixture was stirred at room temperature for 3 h under H₂. After the reaction was completed, the resulting mixture was filtered. The filtrate was concentrated under reduced pressure to afford 4-(4-amino-2-methylphenoxy)-1-ethylpyridin-2-one (2.3 g, crude) as a yellow solid. LCMS (ESI, m/z): [M+H]⁺=245.1.

### Step 3: Synthesis of 1-ethyl-4-[2-methyl-4-[(6-nitroquinazolin-4-yl)amino]phenoxy]pyridin-2-one

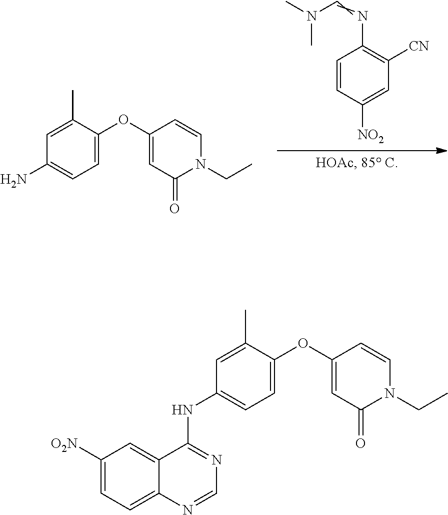

A mixture of 4-(4-amino-2-methylphenoxy)-1-ethylpyridin-2-one (2.3 g, crude) and N′-(2-cyano-4-nitrophenyl)-N,N-dimethylformimidamide (2.5 g, 11.32 mmol) in HOAc (30.0 mL) was stirred at 85° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford 1-ethyl-4-[2-methyl-4-[(6-nitroquinazolin-4-yl)amino]phenoxy]pyridin-2-one (3.0 g, 76%) as a yellow solid. LCMS (ESI, m/z): [M+H]⁺=418.1.

### Step 4: Synthesis of 4-[4-[(6-aminoquinazolin-4-yl)amino]-2-methylphenoxy]-1-ethylpyridin-2-one

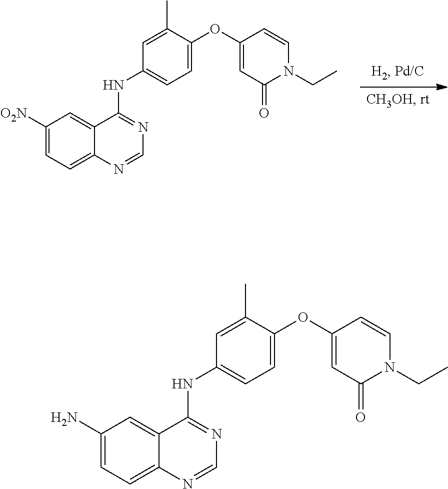 [SMILES: CCn1ccc(Oc2ccc(Nc3ncnc4ccc([N+](=O)[O-])cc34)cc2C)cc1=O ; "CH3OH, rt" ; "H2, Pd/C" ; CCn1ccc(Oc2ccc(Nc3ncnc4ccc(N)cc34)cc2C)cc1=O]

To a solution of 1-ethyl-4-[2-methyl-4-[(6-nitroquinazolin-4-yl)amino]phenoxy]pyridin-2-one (3.0 g, 7.19 mmol) in CH₃OH (50.0 mL) was added Pd/C (0.9 g, dry) at room temperature. The mixture was stirred at room temperature for 3 h under H₂. After the reaction was completed, the resulting mixture was filtered. The filtrate was concentrated under reduced pressure to afford 4-[4-[(6-aminoquinazolin-4-yl)amino]-2-methylphenoxy]-1-ethylpyridin-2-one (2.3 g, crude) as a yellow solid. LCMS (ESI, m/z): [M+H]⁺=388.2.

### Step 5: Synthesis of N-[4-([4-[(1-ethyl-2-oxopyridin-4-yl)oxy]-3-methylphenyl]amino)quinazolin-6-yl]but-2-ynamide

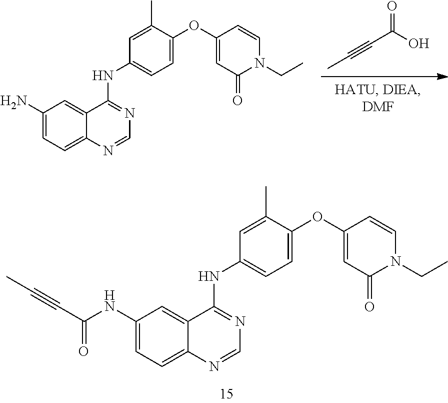

To a solution of 4-[4-[(6-aminoquinazolin-4-yl)amino]-2-methylphenoxy]-1-ethylpyridin-2-one (500.0 mg, 1.29 mmol) in DMF (15.0 mL) was added 2-butynoic acid (162.8 mg, 1.94 mmol), HATU (1472.1 mg, 3.87 mmol) and DIEA (1667.9 mg, 12.91 mmol). The mixture was stirred at room temperature for 1 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) and then purified by Prep-HPLC with the following conditions (Column: YMC-Actus Triart C18 Column, 30×250 mm, 5 um; Mobile Phase A: Water (10 mmol/L NH₄HCO₃), Mobile Phase B: ACN; Flow rate: 60 mL/min; Gradient: 18% B to 42% B in 8 min; 254 nm) to afford N-[4-([4-[(1-ethyl-2-oxopyridin-4-yl)oxy]-3-methylphenyl]amino)quinazolin-6-yl]but-2-ynamide (Compound 15) (23.9 mg, 3%) as a white solid. LCMS (ESI, m/z): [M+H]⁺=454.3. ¹H NMR (400 MHz, DMSO-d₆): δ 10.93 (s, 1H), 9.86 (s, 1H), 8.79 (s, 1H), 8.53 (d, J=8.4 Hz, 1H), 7.81-7.73 (m, 5H), 7.12 (d, J=8.8 Hz, 1H), 6.13-6.11 (m, 1H), 5.34 (d, J=2.4 Hz, 1H), 3.88-3.83 (m, 2H), 2.15-2.03 (m, 6H), 1.25-1.19 (m, 3H).

### Example S16. Synthesis of N-[4-([3-methyl-4-[(5-methylpyrazin-2-yl)oxy]phenyl]amino)quinazolin-6-yl]but-2-ynamide (Compound 16)

### Step 1: Synthesis of 2-methyl-5-(2-methyl-4-nitrophenoxy)pyrazine

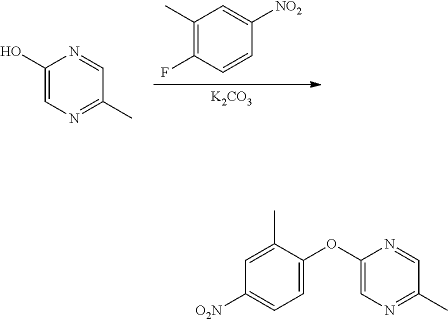

To a solution of 5-methylpyrazin-2-ol (5.0 g, 45.40 mmol) in DMF (50.0 mL) was added K₂CO₃ (12.5 g, 90.81 mmol) and 1-fluoro-2-methyl-4-nitrobenzene (7.0 g, 45.40 mmol). The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 2-methyl-5-(2-methyl-4-nitrophenoxy)pyrazine (5.1 g, 45%) as a light brown solid. LCMS (ESI, m/z): [M+H]⁺=246.0.

### Step 2: Synthesis of 3-methyl-4-[(5-methylpyrazin-2-yl)oxy]aniline

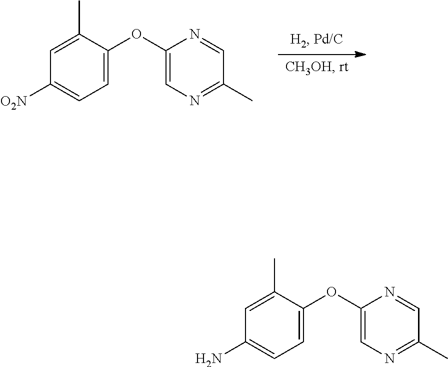 [SMILES: Cc1cnc(Oc2ccc([N+](=O)[O-])cc2C)cn1 ; "CH3OH, rt" ; "H2, Pd/C" ; Cc1cnc(Oc2ccc(N)cc2C)cn1]

To a solution of 2-methyl-5-(2-methyl-4-nitrophenoxy)pyrazine (5.1 g, 20.79 mmol) in MeOH (50.0 mL) was added Pd/C (1.7 g, dry). The resulting mixture was stirred at room temperature for 3 h under H₂. After the reaction was completed, the resulting mixture was filtered. The filtrate was concentrated under vacuum to afford 3-methyl-4-[(5-methylpyrazin-2-yl)oxy]aniline (4.4 g, crude) as a brown oil. LCMS (ESI, m/z): [M+H]⁺=216.0.

### Step 3: Synthesis of N-[3-methyl-4-[(5-methylpyrazin-2-yl)oxy]phenyl]-6-nitroquinazolin-4-amine

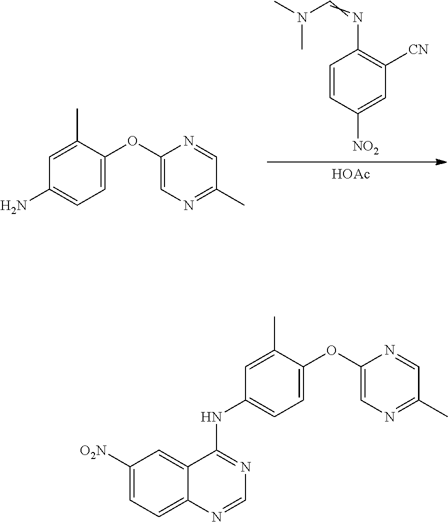 [SMILES: Cc1cnc(Oc2ccc(N)cc2C)cn1 ; CN(C)C=Nc1ccc([N+](=O)[O-])cc1C#N ; Cc1cnc(Oc2ccc(Nc3ncnc4ccc([N+](=O)[O-])cc34)cc2C)cn1 ; "HOAc"]

A mixture of 3-methyl-4-[(5-methylpyrazin-2-yl)oxy]aniline (600.0 mg, crude) and N′-(2-cyano-4-nitrophenyl)-N,N-dimethylformimidamide (608.2 mg, 2.78 mmol) in HOAc (10.0 mL) was stirred at 85° C. for 2 h. After the reaction was completed, the resulting mixture was concentrated under reduced pressure. The residue was purified by flash column chromatography with CH₂Cl₂/MeOH (10/1, v/v) to afford N-[3-methyl-4-[(5-methylpyrazin-2-yl)oxy]phenyl]-6-nitroquinazolin-4-amine (900.0 mg, 83%) as a brown oil. LCMS (ESI, m/z): [M+H]⁺=389.0.

### Step 4: Synthesis of N4-[3-methyl-4-[(5-methylpyrazin-2-yl)oxy]phenyl]quinazoline-4,6-diamine

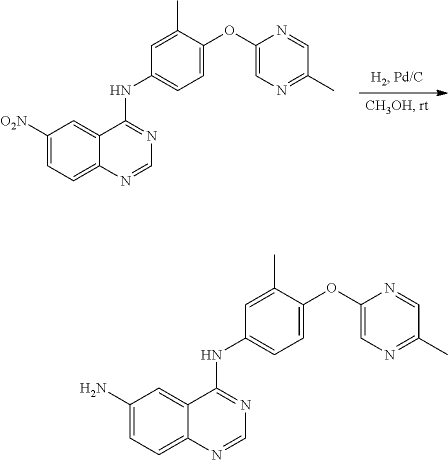 [SMILES: Cc1cnc(Oc2ccc(Nc3ncnc4ccc([N+](=O)[O-])cc34)cc2C)cn1 ; "CH3OH, rt" ; "H2, Pd/C" ; Cc1cnc(Oc2ccc(Nc3ncnc4ccc(N)cc34)cc2C)cn1]

To a solution of N-[3-methyl-4-[(5-methylpyrazin-2-yl)oxy]phenyl]-6-nitroquinazolin-4-amine (900.0 mg, 2.31 mmol) in MeOH (20.0 mL) was added Pd/C (300.0 mg, dry). The resulting mixture was stirred at room temperature for 3 h under H₂. After the reaction was completed, the resulting mixture was filtered. The filtrate was concentrated under vacuum. The residue was purified by flash column chromatography with CH₂Cl₂/MeOH (10/1, v/v) to afford N4-[3-methyl-4-[(5-methylpyrazin-2-yl)oxy]phenyl]quinazoline-4,6-diamine (350.0 mg, 42%) as a yellow solid. LCMS (ESI, m/z): [M+H]⁺=359.0.

### Step 5: Synthesis of N-[4-([3-methyl-4-[(5-methylpyrazin-2-yl)oxy]phenyl]amino)quinazolin-6-yl]but-2-ynamide (Compound 16)

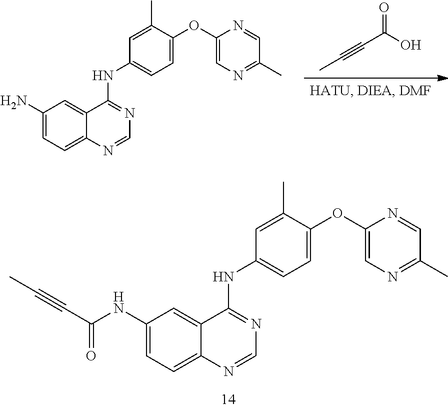

To a mixture of N4-[3-methyl-4-[(5-methylpyrazin-2-yl)oxy]phenyl]quinazoline-4,6-diamine (300.0 mg, 0.83 mmol) and 2-butynoic acid (105.5 mg, 1.25 mmol) in DMF (10.0 mL) was added DIEA (649.0 mg, 5.02 mmol) and HATU (954.8 mg, 2.51 mmol) at room temperature. The resulting mixture was stirred at room temperature for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with ACN in water (5% to 100% gradient in 40 min) and then purified by Prep-HPLC with the following conditions (Column:) (Bridge Prep OBD C18 Column, 30×150 mm, 5 um; Mobile Phase A: Water (10 mmol/L NH₄HCO₃), Mobile Phase B: ACN; Flow rate: 60 mL/min; Gradient: 32% B to 42% B in 8 min; 254/220 nm) to afford N-[4-([3-methyl-4-[(5-methylpyrazin-2-yl)oxy]phenyl]amino)quinazolin-6-yl]but-2-ynamide (Compound 16) (32.5 mg, 9%) as a light yellow solid. LCMS (ESI, m/z): [M+H]⁺=425.2. ¹H NMR (300 MHz, DMSO-d₆): δ 10.91 (s, 1H), 9.81 (s, 1H), 8.73 (s, 1H), 8.51 (s, 1H), 8.42 (d, J=1.2 Hz, 1H), 8.05 (d, J=0.6 Hz, 1H), 7.81-7.62 (m, 4H), 7.10 (d, J=8.4 Hz, 1H), 2.44 (s, 3H), 2.12-2.09 (m, 6H).

### Example S17. Synthesis of N-[4-[(4-[[1-(difluoromethyl)-2-oxopyridin-4-yl]oxy]-3-methylphenyl)amino]quinazolin-6-yl]but-2-ynamide (Compound 17)

### Step 1a: Synthesis of 2-chloro-4-(2-methyl-4-nitrophenoxy)pyridine

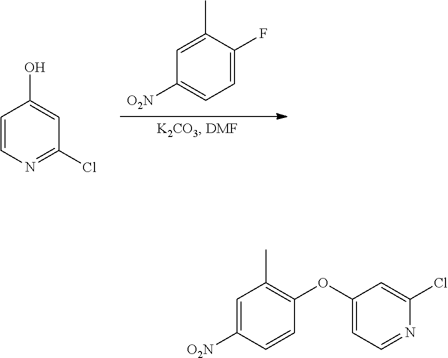

A mixture of 2-chloropyridin-4-ol (5.0 g, 38.59 mmol), 1-fluoro-2-methyl-4-nitrobenzene (7.1 g, 46.31 mmol) and K₂CO₃ (10.6 g, 77.19 mmol) in DMF (50.0 mL) was stirred at 50° C. for 16 h. After the reaction was completed, the resulting mixture was filtered. The filtrate was concentrated under vacuum. The residue was purified by reverse phase flash column chromatography with ACN in water (5% to 100% gradient in 40 min) to afford 2-chloro-4-(2-methyl-4-nitrophenoxy)pyridine (2.9 g, 28%) as a yellow solid. LCMS (ESI, m/z): [M+H]⁺=265.0.

### Step 1b: Alternative Synthesis of 2-chloro-4-(2-methyl-4-nitrophenoxy)pyridine

In an alternative to Step 1a: A mixture of 2-chloropyridin-4-ol (25.0 g), 1-fluoro-2-methyl-4-nitrobenzene (33.0 g) and K₂CO₃ (53.5 g) in DMF (75.0 mL) was stirred at 80° C. for 24 hours. After the reaction was completed, the resulting mixture was diluted with EA (500 mL) and washed with water (200 mL×5), the organic layer was concentrated under vacuum to afford 2-chloro-4-(2-methyl-4-nitrophenoxy)pyridine (50.0 g) as a yellow solid. LCMS (ESI, m/z): [M+H]⁺=265.0.

### Step 2a: Synthesis of 1-(difluoromethyl)-4-(2-methyl-4-nitrophenoxy)pyridin-2-one

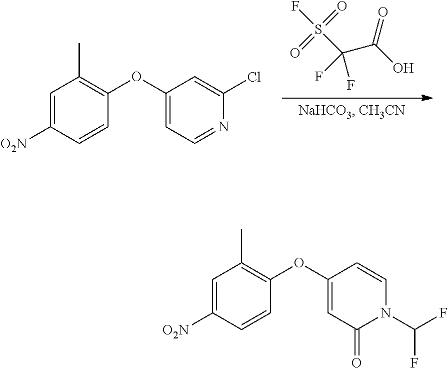

To a solution of 2-chloro-4-(2-methyl-4-nitrophenoxy)pyridine (2.8 g, 10.57 mmol) in CH₃CN (30.0 mL) was added NaHCO₃ (1.7 g, 21.15 mmol) at room temperature. The resulting mixture was stirred at 80° C. for 30 min. Then a solution of difluoro(sulfo)acetic acid (1.8 g, 10.55 mmol) in CH₃CN (10.0 mL) was added to the mixture at room temperature. The mixture was stirred at 80° C. for another 2 h. After the reaction was completed, the resulting mixture was concentrated under reduced pressure. The residue was purified by flash column chromatography with petroleum ether/ethyl acetate (1/1, v/v) to afford 1-(difluoromethyl)-4-(2-methyl-4-nitrophenoxy)pyridin-2-one (1.5 g, 47%) as a light yellow solid. LCMS (ESI, m/z): [M+H]⁺=297.0.

### Step 2b: Alternative Synthesis of 1-(difluoromethyl)-4-(2-methyl-4-nitrophenoxy)pyridin-2-one

In an alternative to Step 2a: To a solution of 2-chloro-4-(2-methyl-4-nitrophenoxy)pyridine (29.0 g) in CH₃CN (300.0 mL) was added NaHCO₃ (18.6 g) at room temperature. The resulting mixture was stirred at 80° C. for 30 min. Then difluoro(sulfo)acetic acid (59.0 g) was added to the mixture at 0° C. The mixture was stirred at 80° C. for another 2 hours. After the reaction was completed, the resulting mixture was filtered. The filtrate was concentrated under reduced pressure. The residue was diluted with EA (500 mL) and washed with water (200 mL×3), the organic layer was concentrated under vacuum to afford 1-(difluoromethyl)-4-(2-methyl-4-nitrophenoxy)pyridin-2-one (30.0 g) as a yellow solid. LCMS (ESI, m/z): [M+H]⁺=297.0.

### Step 3a: Synthesis of 4-(4-amino-2-methylphenoxy)-1-(difluoromethyl)pyridin-2(1H)-one

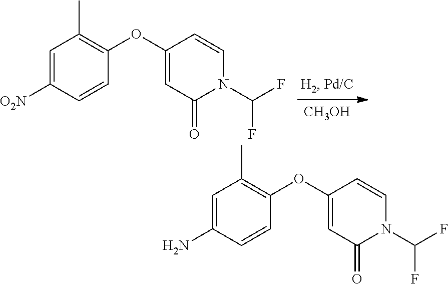

To a solution of 1-(difluoromethyl)-4-(2-methyl-4-nitrophenoxy)pyridin-2-one (1.5 g, 5.06 mmol) in MeOH (20.0 mL) was added Pd/C (500.0 mg, dry). The resulting mixture was stirred at room temperature for 3 h under H₂. After the reaction was completed, the resulting mixture was filtered. The filtrate was concentrated under vacuum to afford 4-(4-amino-2-methylphenoxy)-1-(difluoromethyl)pyridin-2(1H)-one (1.3 g, crude) as a brown solid. LCMS (ESI, m/z): [M+H]⁺=267.0.

### Step 3b: Alternative Synthesis of 4-(4-amino-2-methylphenoxy)-1-(difluoromethyl)pyridin-2(1H)-one

In an alternative to Step 3a: To a solution of 1-(difluoromethyl)-4-(2-methyl-4-nitrophenoxy)pyridin-2-one (6.0 g) in MeOH (80.0 mL) was added Pd/C (2.0 g, dry). The resulting mixture was stirred at room temperature for 3 h under H₂. After the reaction was completed, the resulting mixture was filtered. The filtrate was concentrated under vacuum to afford 4-(4-amino-2-methylphenoxy)-1-(difluoromethyl)pyridin-2(1H)-one (4.4 g, crude) as a brown oil. LCMS (ESI, m/z): [M+H]⁺=267.0.

### Step 4a: Synthesis of 1-(difluoromethyl)-4-[2-methyl-4-[(6-nitroquinazolin-4-yl)amino]phenoxy]pyridin-2-one

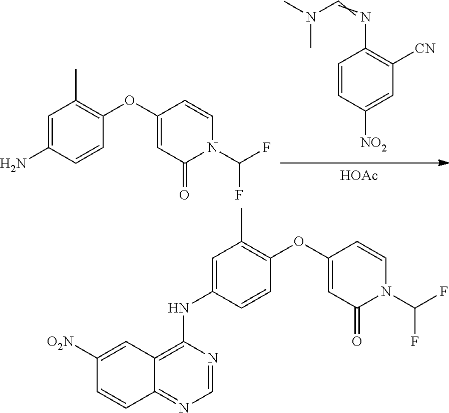

A mixture of 4-(4-amino-2-methylphenoxy)-1-(difluoromethyl)pyridin-2(1H)-one (1.2 g, crude) and N′-(2-cyano-4-nitrophenyl)-N,N-dimethylformimidamide (983.5 mg, 4.50 mmol) in HOAc (20.0 mL) was stirred at 85° C. for 2 h. After the reaction was completed, the resulting mixture was concentrated under reduced pressure. The residue was purified by flash column chromatography with CH₂Cl₂/MeOH (10/1, v/v) to afford 1-(difluoromethyl)-4-[2-methyl-4-[(6-nitroquinazolin-4-yl)amino]phenoxy]pyridin-2-one (1.1 g, 60%) as a yellow solid. LCMS (ESI, m/z): [M+H]⁺=440.0.

### Step 4b: Alternative Synthesis of 1-(difluoromethyl)-4-[2-methyl-4-[(6-nitroquinazolin-4-yl)amino]phenoxy]pyridin-2-one

In an alternative to Step 4a: A mixture of 4-(4-amino-2-methylphenoxy)-1-(difluoromethyl)pyridin-2(1H)-one (23.7 g, crude) and N′-(2-cyano-4-nitrophenyl)-N,N-dimethylformimidamide (19.1 g) in AcOH (350 mL) was stirred at 85° C. for 2 hours. After the reaction was completed, the resulting mixture was concentrated under reduced pressure. The residue was purified by flash column chromatography with PE/EA (35/65, v/v) to afford 1-(difluoromethyl)-4-[2-methyl-4-[(6-nitroquinazolin-4-yl)amino]phenoxy]pyridin-2-one (21 g) as a yellow solid. LCMS (ESI, m/z): [M+H]⁺=440.0.

### Step 5a: Synthesis of 4-[4-[(6-aminoquinazolin-4-yl)amino]-2-methylphenoxy]-1-(difluoromethyl)pyridin-2-one

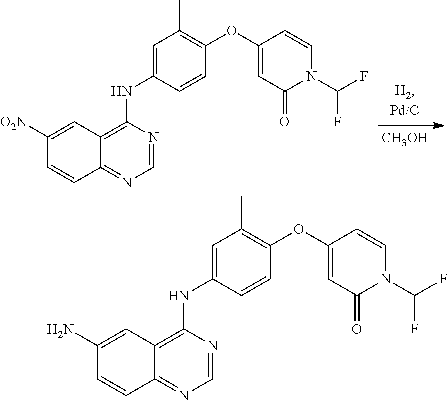

To a solution of 1-(difluoromethyl)-4-[2-methyl-4-[(6-nitroquinazolin-4-yl)amino]phenoxy]pyridin-2-one (1.1 g, 2.50 mmol) in MeOH (25.0 mL) was added Pd/C (350.0 mg, dry). The resulting mixture was stirred at room temperature for 3 h under H₂. After the reaction was completed, the resulting mixture was filtered. The filtrate was concentrated under vacuum to afford 4-[4-[(6-aminoquinazolin-4-yl)amino]-2-methylphenoxy]-1-(difluoromethyl)pyridin-2-one (900.0 mg, crude) as a brown solid. LCMS (ESI, m/z): [M+H]⁺=410.1.

### Step 5b: Alternative Synthesis of 4-[4-[(6-aminoquinazolin-4-yl)amino]-2-methylphenoxy]-1-(difluoromethyl)pyridin-2-one

In an alternative to Step 5a: To a solution of 1-(difluoromethyl)-4-[2-methyl-4-[(6-nitroquinazolin-4-yl)amino]phenoxy]pyridin-2-one (21 g) in MeOH (300 mL) was added Pd/C (7 g, dry). The resulting mixture was stirred at room temperature for 3 h under H₂. After the reaction was completed, the resulting mixture was filtered. The filtrate was concentrated under vacuum to afford 4-[4-[(6-aminoquinazolin-4-yl)amino]-2-methylphenoxy]-1-(difluoromethyl)pyridin-2-one (10.1 g) as a pale green solid. LCMS (ESI, m/z): [M+H]⁺=410.1.

### Step 6a: Synthesis of N-[4-[(4-[[1-(difluoromethyl)-2-oxopyridin-4-yl]oxy]-3-methylphenyl)amino]quinazolin-6-yl]but-2-ynamide (Compound 17)

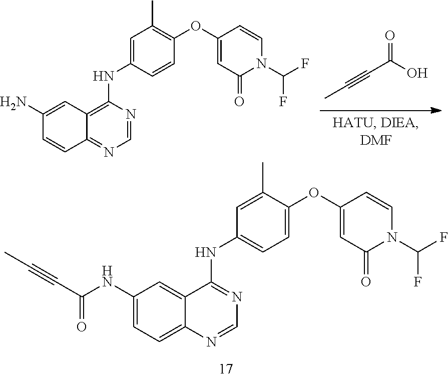

To a solution of 4-[4-[(6-aminoquinazolin-4-yl)amino]-2-methylphenoxy]-1-(difluoromethyl)pyridin-2-one (400.0 mg, 0.97 mmol) in DMF (6.0 mL) was added 2-butynoic acid (98.5 mg, 1.17 mmol), DIEA (378.8 mg, 2.93 mmol) and HATU (557.2 mg, 1.46 mmol) at room temperature. The resulting mixture was stirred at room temperature for 1 h. After the reaction was completed, the reaction mixture was purified by reverse phase flash column chromatography with ACN in water (5% to 100% gradient in 40 min) and then purified by Prep-HPLC with the following conditions (Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 41% B to 50% B in 9 min; 254/220 nm) to afford N-[4-[(4-[[1-(difluoromethyl)-2-oxopyridin-4-yl]oxy]-3-methylphenyl)amino]quinazolin-6-yl]but-2-ynamide (Compound 17) (36.1 mg, 7%) as a white solid. LCMS (ESI, m/z): [M+H]⁺=476.2. ¹H NMR (400 MHz, DMSO-d₆): δ 10.93 (s, 1H), 9.86 (s, 1H), 8.74 (s, 1H), 8.53 (s, 1H), 7.93-7.64 (m, 6H), 7.16 (d, J=8.8 Hz, 1H), 6.37-6.34 (m, 1H), 5.35 (d, J=2.4 Hz, 1H), 2.15 (s, 3H), 2.08 (s, 3H).

### Step 6b: Alternative Synthesis of N-[4-[(4-[[1-(difluoromethyl)-2-oxopyridin-4-yl]oxy]-3-methylphenyl)amino]quinazolin-6-yl]but-2-ynamide (Compound 17)

In an alternative to Step 6a: To a solution of 4-[4-[(6-aminoquinazolin-4-yl)amino]-2-methylphenoxy]-1-(difluoromethyl)pyridin-2-one (5 g) and 2-butynoic acid (2.1 g) in DCM (50 mL) was added Et₃N (6.25 g) at room temperature. Then T₃P (23 g) was added at 0° C. and stirred at room temperature for 1 hour. The solvent was concentrated under vacuum. The residue was purified by flash chromatography on silica gel eluting with DCM/MeOH (96/4) to afford N-[4-[(4-[[1-(difluoromethyl)-2-oxopyridin-4-yl]oxy]-3-methylphenyl)amino]quinazolin-6-yl]but-2-ynamide (Compound 17) (1.1862 g) as a pale yellow solid. LCMS (ESI, m/z): [M+H]⁺=476.2. LCMS (ESI, m/z): [M+H]⁺=476.2, RT=1.367 min; 1H NMR (300 MHz, DMSO-d₆): δ 10.93 (s, 1H), 9.86 (s, 1H), 8.74 (s, 1H), 8.53 (s, 1H), 7.93-7.64 (m, 6H), 7.16 (d, J=8.8 Hz, 1H), 6.37-6.34 (m, 1H), 5.35 (d, J=2.4 Hz, 1H), 2.15 (s, 3H), 2.08 (s, 3H).

### Example S18. Synthesis of N-[4-[(3-methyl-4-[[1,2,4]triazolo[4,3-a]pyridin-7-yloxy]phenyl)amino]quinazolin-6-yl]but-2-ynamide (Compound 18)

### Step 1: Synthesis of 4-(benzyloxy)-2-chloropyridine

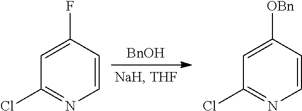

To a solution of benzyl alcohol (8.2 g, 76.03 mmol) in DMF (50.0 mL) was added NaH (3.7 g, 60%) at 0° C. under N₂. The mixture was stirred at 0° C. for 20 min under N₂. Then 2-chloro-4-fluoropyridine (10.0 g, 76.03 mmol) was added to the mixture. The mixture was stirred at room temperature for another 1 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (50/50, v/v) to afford 4-(benzyloxy)-2-chloropyridine (13.0 g, 78%) as a white solid. LCMS (ESI, m/z): [M+H]⁺=220.0.

### Step 2: Synthesis of 4-(benzyloxy)-2-hydrazinylpyridine

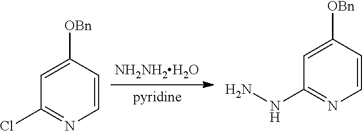

A mixture of 4-(benzyloxy)-2-chloropyridine (12.0 g, 54.63 mmol) and hydrazine (450.0 mL, 80%) in pyridine (1200.0 mL) was stirred at 110° C. for 16 h. After the reaction was completed, the resulting mixture was concentrated under vacuum to afford 4-(benzyloxy)-2-hydrazinylpyridine (11.0 g, crude). LCMS (ESI, m/z): [M+H]⁺=216.1.

### Step 3: Synthesis of 7-(benzyloxy)-[1,2,4]triazolo[4,3-a]pyridine

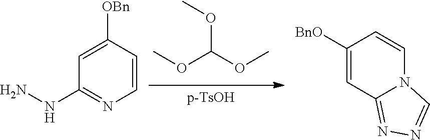

A mixture of 4-(benzyloxy)-2-hydrazinylpyridine (11.0 g, 51.10 mmol) and p-Toluenesulfonic acid (6.1 g, 0.04 mmol) in trimethyl orthoformate (60.0 mL) was stirred at 60° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (90/10, v/v) to afford 7-(benzyloxy)-[1,2,4]triazolo[4,3-a]pyridine (6.7 g, 58%) as a red oil. LCMS (ESI, m/z): [M+H]⁺=226.1.

### Step 4: Synthesis of [1,2,4]triazolo[4,3-a]pyridin-7-ol

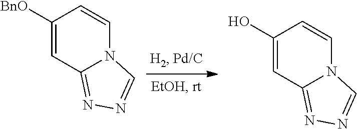 [SMILES: c1ccc(COc2ccn3cnnc3c2)cc1 ; Oc1ccn2cnnc2c1]

To a solution of 7-(benzyloxy)-[1,2,4]triazolo[4,3-a]pyridine (5.7 g, 25.30 mmol) in CH₃OH (50.0 mL) was added Pd/C (1.7 g, dry) at room temperature under N₂. The mixture was stirred at room temperature for 3 h under H₂. After the reaction was completed, the resulting mixture was filtered. The filtrate was concentrated under reduced pressure to afford [1,2,4]triazolo[4,3-a]pyridin-7-ol (4.2 g, crude) as a yellow solid. LCMS (ESI, m/z): [M+H]⁺=136.0.

### Step 5: Synthesis of 7-(2-methyl-4-nitrophenoxy)-[1,2,4]triazolo[4,3-a]pyridine

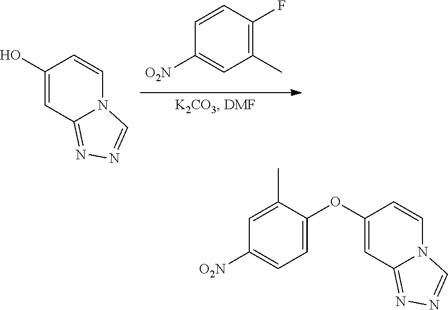 [SMILES: Oc1ccn2cnnc2c1 ; Cc1cc([N+](=O)[O-])ccc1F ; Cc1cc([N+](=O)[O-])ccc1Oc1ccn2cnnc2c1]

A mixture of [1,2,4]triazolo[4,3-a]pyridin-7-ol (1.5 g, 11.10 mmol), 1-fluoro-2-methyl-4-nitrobenzene (2.1 g, 13.32 mmol) and K₂CO₃ (3.1 g, 22.20 mmol) in DMF (40.0 mL) was stirred at 50° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford 7-(2-methyl-4-nitrophenoxy)-[1,2,4]triazolo[4,3-a]pyridine (644.0 mg, 21%) as a yellow solid. LCMS (ESI, m/z): [M+H]⁺=271.1.

### Step 6: Synthesis of 3-methyl-4-[[1,2,4]triazolo[4,3-a]pyridin-7-yloxy]aniline

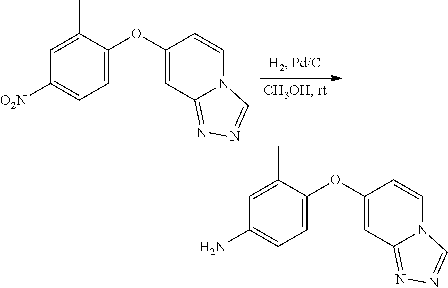

To a solution of 7-(2-methyl-4-nitrophenoxy)-[1,2,4]triazolo[4,3-a]pyridine (750.0 mg, 2.78 mmol) in CH₃OH (40.0 mL) was added Pd/C (230.4 mg, dry). The mixture was stirred at room temperature for 16 h under H₂. After the reaction was completed, the resulting mixture was filtered. The filtrate was concentrated under reduced pressure to afford 3-methyl-4-[[1,2,4]triazolo[4,3-a]pyridin-7-yloxy]aniline (700.0 mg, crude) as a yellow solid. LCMS (ESI, m/z): [M+H]⁺=241.1.

### Step 7: Synthesis of N-(3-methyl-4-[[1,2,4]triazolo[4,3-a]pyridin-7-yloxy]phenyl)-6-nitroquinazolin-4-amine

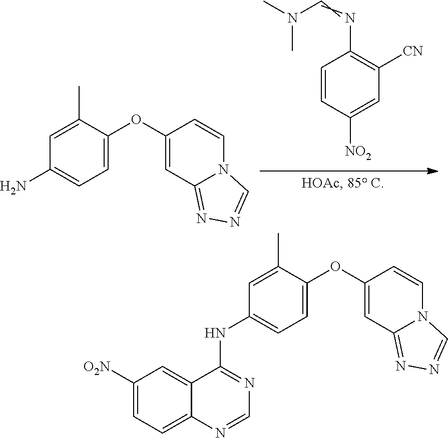

A solution of 3-methyl-4-[[1,2,4]triazolo[4,3-a]pyridin-7-yloxy]aniline (700.0 mg, crude) and N′-(2-cyano-4-nitrophenyl)-N,N-dimethylformimidamide (762.9 mg, 3.50 mmol) in CH₃COOH (17.5 mL) was stirred at 85° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (89/11, v/v) to afford N-(3-methyl-4-[[1,2,4]triazolo[4,3-a]pyridin-7-yloxy]phenyl)-6-nitroquinazolin-4-amine (1.1 g, 91%) as a yellow solid. LCMS (ESI, m/z): [M+H]⁺=414.1.

### Step 8: Synthesis of N4-(3-methyl-4-[[1,2,4]triazolo[4,3-a]pyridin-7-yloxy]phenyl)quinazoline-4,6-diamine

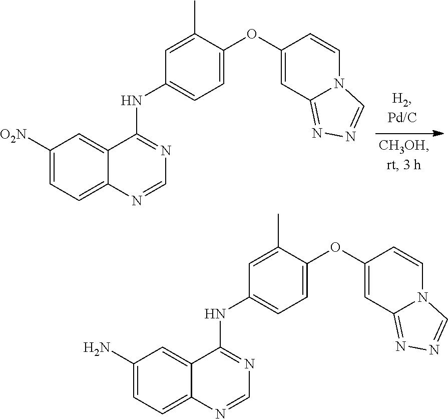 [SMILES: Cc1cc(Nc2ncnc3ccc([N+](=O)[O-])cc23)ccc1Oc1ccn2cnnc2c1 ; "CH3OH,rt, 3 h" ; Cc1cc(Nc2ncnc3ccc(N)cc23)ccc1Oc1ccn2cnnc2c1]

To a solution of N-(3-methyl-4-[[1,2,4]triazolo[4,3-a]pyridin-7-yloxy]phenyl)-6-nitroquinazolin-4-amine (500.0 mg, 1.21 mmol) in CH₃OH (15.0 mL) was added Pd/C (150.0 mg, dry) at room temperature. The mixture was stirred at room temperature for 4 h under H₂. After the reaction was completed, the resulting mixture was filtered. The filtrate was concentrated under reduced pressure to afford N4-(3-methyl-4-[[1,2,4]triazolo[4,3-a]pyridin-7-yloxy]phenyl)quinazoline-4,6-diamine (480.0 mg, crude) as a yellow solid. LCMS (ESI, m/z): [M+H]⁺=384.1.

### Step 9: Synthesis of N-[4-[(3-methyl-4-[[1,2,4]triazolo[4,3-a]pyridin-7-yloxy]phenyl)amino]quinazolin-6-yl]but-2-ynamide (Compound 18)

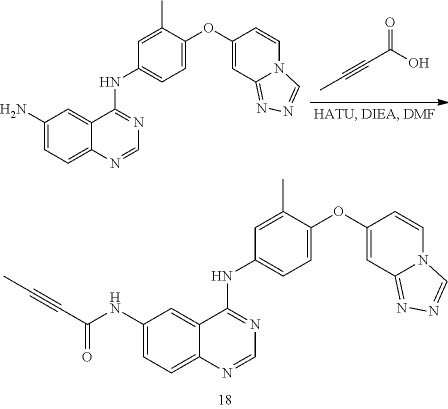

A mixture of N4-(3-methyl-4-[[1,2,4]triazolo[4,3-a]pyridin-7-yloxy]phenyl)quinazoline-4,6-diamine (300.0 mg, 0.78 mmol), 2-butynoic acid (78.9 mg, 0.94 mmol), HATU (892.5 mg, 2.35 mmol) and DIEA (1011.3 mg, 7.82 mmol) in DMF (20.0 mL) was stirred at room temperature for 1 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (84/16, v/v) and then purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 7% B to 23% B in 7 min; 254/220 nm) to afford N-[4-[(3-methyl-4-[[1,2,4]triazolo[4,3-a]pyridin-7-yloxy]phenyl)amino]quinazolin-6-yl]but-2-ynamide (Compound 18) (82.4 mg, 23%) as a white solid. LCMS (ESI, m/z): [M+H]⁺=450.2. ¹H NMR (300 MHz, DMSO-d₆): δ 10.93 (s, 1H), 9.88 (s, 1H), 9.13 (s, 1H), 8.76 (s, 1H), 8.60-8.55 (m, 2H), 7.82-7.75 (m, 4H), 7.18 (d, J=8.7 Hz, 1H), 6.94-6.91 (m, 1H), 6.60 (s, 1H), 2.19 (s, 3H), 2.09 (s, 3H).

### Example S19. Synthesis of N-methyl-N-[4-[(3-methyl-4-[[1,2,4]triazolo[1,5-a]pyridin-7-yloxy]phenyl)amino]quinazolin-6-yl]but-2-ynamide (Compound 19)

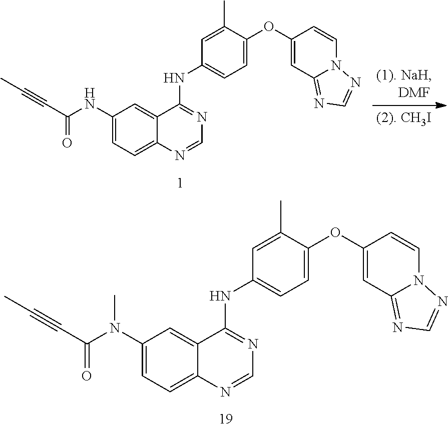 [SMILES: CC#CC(=O)Nc1ccc2ncnc(Nc3ccc(Oc4ccn5ncnc5c4)c(C)c3)c2c1 ; CC#CC(=O)N(C)c1ccc2ncnc(Nc3ccc(Oc4ccn5ncnc5c4)c(C)c3)c2c1]

To a solution of N-[4-[(3-methyl-4-[[1,2,4]triazolo[1,5-a]pyridin-7-yloxy]phenyl)amino]quinazolin-6-yl]but-2-ynamide (Compound 1) (247.0 mg, 0.55 mmol) in DMF (5.0 mL) was added NaH (44.0 mg, 60%) at 0° C. under N₂. The mixture was stirred at 0° C. for 30 min. Then CH₃I (78.0 mg, 0.55 mmol) was added to the mixture at 0° C. under N2. The mixture was stirred at room temperature for 1 h. After the reaction was completed, the reaction mixture was quenched with water and extracted with DCM. The combined organic layer was washed with brine, dried over anhydrous sodium sulfate and filtered. The filtrate was concentrated under vacuum. The residue was purified by Prep-HPLC with the following conditions: Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 19% B to 49% B in 10 min; 254 nm; RT1:9.5 min to afford N-methyl-N-[4-[(3-methyl-4-[[1,2,4]triazolo[1,5-a]pyridin-7-yloxy]phenyl)amino]quinazolin-6-yl]but-2-ynamide (Compound 19) (16.8 mg, 6%) as a white solid. LCMS (ESI, m/z): [M+H]⁺=464.2. ¹H NMR (300 MHz, DMSO-d₆): δ 9.87 (s, 1H), 8.95 (d, J=7.5 Hz, 1H), 8.66 (s, 2H), 8.39 (s, 1H), 7.89-7.84 (m, 4H), 7.26-7.22 (m, 1H), 7.06-7.03 (m, 1H), 6.81 (d, J=2.4 Hz, 1H), 3.67 (s, 1H), 3.35 (s, 2H), 2.21 (s, 3H), 2.16 (s, 1H), 1.75 (s, 2H).

### Example S20. Synthesis of N-[4-([4-[(6-methylpyridin-3-yl)methyl]phenyl]amino)quinazolin-6-yl]but-2-ynamide (Compound 20)

### Step 1: Synthesis of 2-methyl-5-[(4-nitrophenyl)methyl]pyridine

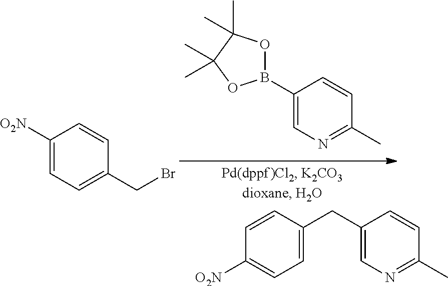

To a solution of nitrobenzyl bromide (2.0 g, 9.26 mmol) in 1,4-dioxane/H₂O (10.0/2.0 mL) was added 2-methyl-5-(4,4,5,5-tetramethyl-1,3,2-dioxaborolan-2-yl)pyridine (2.4 g, 11.11 mmol), K₂CO₃ (3.8 g, 27.77 mmol) and Pd(dppf)Cl₂ (677.4 mg, 0.93 mmol) at room temperature under N₂. The resulting mixture was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (9/1, v/v) to afford 2-methyl-5-[(4-nitrophenyl)methyl]pyridine (780.0 mg, 36%) as a yellow solid. LCMS (ESI, m/z): [M+H]⁺=229.1.

### Step 2: Synthesis of 4-[(6-methylpyridin-3-yl)methyl]aniline

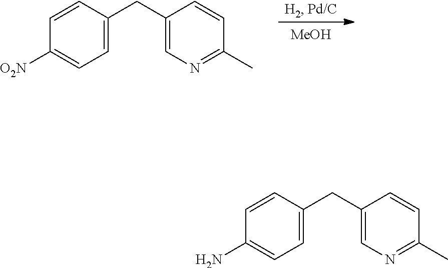 [SMILES: "MeOH" ; Cc1ccc(Cc2ccc([N+](=O)[O-])cc2)cn1 ; "H2, Pd/C" ; Cc1ccc(Cc2ccc(N)cc2)cn1]

To a solution of 2-methyl-5-[(4-nitrophenyl)methyl]pyridine (780.0 mg, 3.42 mmol) in CH₃OH (10.0 mL) was added Pd/C (363.7 mg, dry) at room temperature. The resulting mixture was stirred at room temperature for 2 h under H₂. After the reaction was completed, the resulting mixture was filtered. The filtrate was concentrated under reduced pressure. The residue was purified by flash column chromatography with CH₂Cl₂/CH₃OH (95/5, v/v) to afford 4-[(6-methylpyridin-3-yl)methyl]aniline (300.0 mg, 44%) as a yellow solid. LCMS (ESI, m/z): [M+H]⁺=199.1.

### Step 3: Synthesis of N-[4-[(6-methylpyridin-3-yl)methyl]phenyl]-6-nitroquinazolin-4-amine

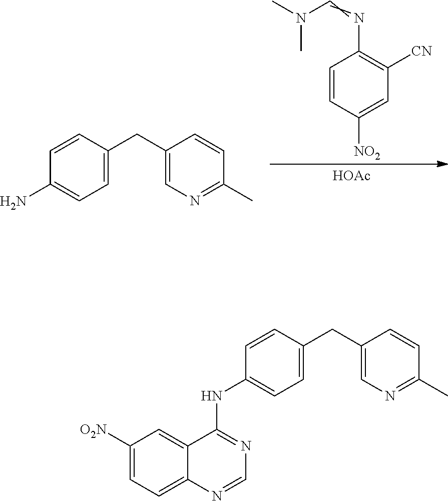 [SMILES: Cc1ccc(Cc2ccc(N)cc2)cn1 ; CN(C)C=Nc1ccc([N+](=O)[O-])cc1C#N ; Cc1ccc(Cc2ccc(Nc3ncnc4ccc([N+](=O)[O-])cc34)cc2)cn1 ; "HOAc"]

To a solution of 4-[(6-methylpyridin-3-yl)methyl]aniline (200.0 mg, 1.01 mmol) in HOAc (5.0 mL) was added N′-(2-cyano-4-nitrophenyl)-N,N-dimethylformimidamide (220.1 mg, 1.01 mmol) at room temperature. The resulting mixture was stirred at 85° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (95/5, v/v) to afford N-[4-[(6-methylpyridin-3-yl)methyl]phenyl]-6-nitroquinazolin-4-amine (350.0 mg, 93%) as a yellow solid. LCMS (ESI, m/z): [M+H]⁺=372.1.

### Step 4: Synthesis of N4-[4-[(6-methylpyridin-3-yl)methyl]phenyl]quinazoline-4,6-diamine

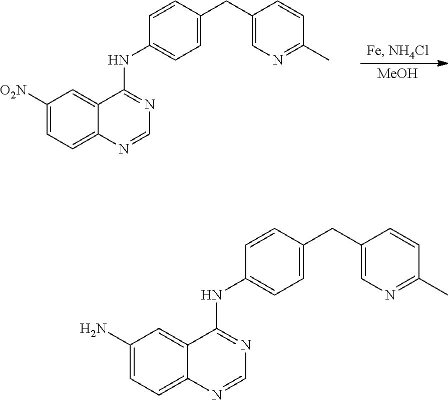

To a solution of N-[4-[(6-methylpyridin-3-yl)methyl]phenyl]-6-nitroquinazolin-4-amine (350.0 mg, 0.94 mmol) in CH₃OH/H₂O (5.0/1.0 mL) was added NH₄Cl (504.1 mg, 9.42 mmol) and Fe (526.3 mg, 9.42 mmol) at room temperature. The resulting mixture was stirred at 80° C. for 1 h. After the reaction was completed, the resulting mixture was filtered. The filtrate was concentrated under reduced pressure. The residue was purified by flash column chromatography with CH₂Cl₂/CH₃OH (4/1, v/v) to afford N4-[4-[(6-methylpyridin-3-yl)methyl]phenyl]quinazoline-4,6-diamine (200.0 mg, 62%) as a yellow oil. LCMS (ESI, m/z): [M+H]⁺=342.1.

### Step 5: Synthesis of N-[4-([4-[(6-methylpyridin-3-yl)methyl]phenyl]amino)quinazolin-6-yl]but-2-ynamide (Compound 20)

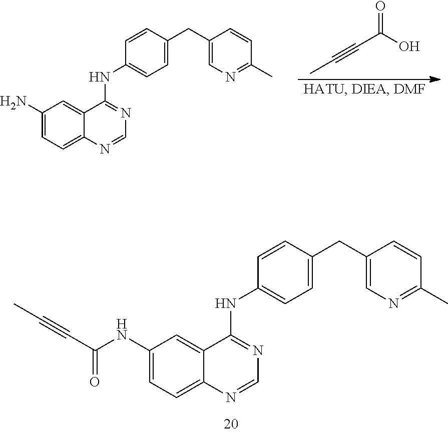

To a solution of N4-[4-[(6-methylpyridin-3-yl)methyl]phenyl]quinazoline-4,6-diamine (200.0 mg, 0.59 mmol) in DMF (5.0 mL) was added 2-butynoic acid (64.0 mg, 0.76 mmol), DIEA (757.1 mg, 5.86 mmol) and HATU (356.4 mg, 0.94 mmol) at room temperature. The resulting mixture was stirred at room temperature for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse flash column chromatography with acetonitrile/water (25/75, v/v) and then purified by Prep-HPLC with the following conditions (Column: YMC-Actus Triart C18, 30×250, 5 um; Mobile Phase A: Water (10 mmol/L NH₄HCO₃), Mobile Phase B: ACN; Flow rate: 60 mL/min; Gradient: 38% B to 47% B in 7 min; 254 nm) to afford N-[4-([4-[(6-methylpyridin-3-yl)methyl]phenyl]amino)quinazolin-6-yl]but-2-ynamide (Compound 20) (16.5 mg, 6%) as a white solid. LCMS (ESI, m/z): [M+H]⁺=408.2. ¹H NMR (300 MHz, DMSO-d₆): δ 10.90 (s, 1H), 9.76 (s, 1H), 8.70 (s, 1H), 8.46 (s, 1H), 8.38 (d, J=2.1 Hz, 1H), 7.79-7.66 (m, 4H), 7.54-7.51 (m, 1H), 7.25-7.16 (m, 3H), 3.91 (s, 2H), 2.42 (s, 3H), 2.08 (s, 3H).

### Example S21. Synthesis of N-(4-((4-([1,2,4]triazolo[1,5-a]pyridin-7-yloxy)-3-methylphenyl)amino)quinazolin-6-yl)-4-hydroxybut-2-ynamide (Compound 21)

### Step 1: Synthesis of N-[4-[(3-methyl-4-[[1,2,4]triazolo[1,5-a]pyridin-7-yloxy]phenyl)amino]quinazolin-6-yl]-4-(oxan-2-yloxy)but-2-ynamide

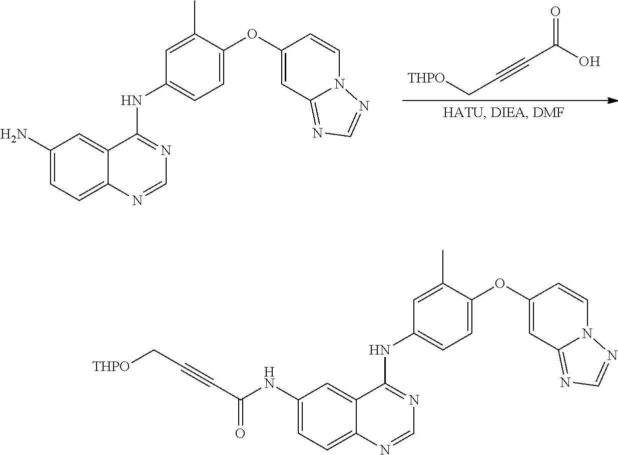

To a solution of N4-(3-methyl-4-[[1,2,4]triazolo[1,5-a]pyridin-7-yloxy]phenyl)quinazoline-4,6-diamine (400.0 mg, 1.04 mmol) in DMF (6.0 mL) was added 4-(oxan-2-yloxy)but-2-ynoic acid (384.3 mg, 2.08 mmol), HATU (1.1 g, 3.13 mmol) and DIEA (809.0 mg, 6.26 mmol). The mixture was stirred at room temperature for 1 h. After the reaction was completed, the resulting mixture was diluted with water and extracted with CH₂Cl₂. The combined organic layer was washed with brine, dried over anhydrous Na₂SO₄ and filtered. The filtrate was evaporated in vacuo. The residue was purified by flash column chromatography with CH₂Cl₂/MeOH (10/1, v/v) to afford N-[4-[(3-methyl-4-[[1,2,4]triazolo[1,5-a]pyridin-7-yloxy]phenyl)amino]quinazolin-6-yl]-4-(oxan-2-yloxy)but-2-ynamide (140.0 mg, 24%) as an off-white solid. LCMS (ESI, m/z): [M+H]⁺=550.2.

### Step 2: Synthesis of N-(4-((4-([1,2,4]triazolo[1,5-a]pyridin-7-yloxy)-3-methylphenyl)amino)quinazolin-6-yl)-4-hydroxybut-2-ynamide (Compound 21)

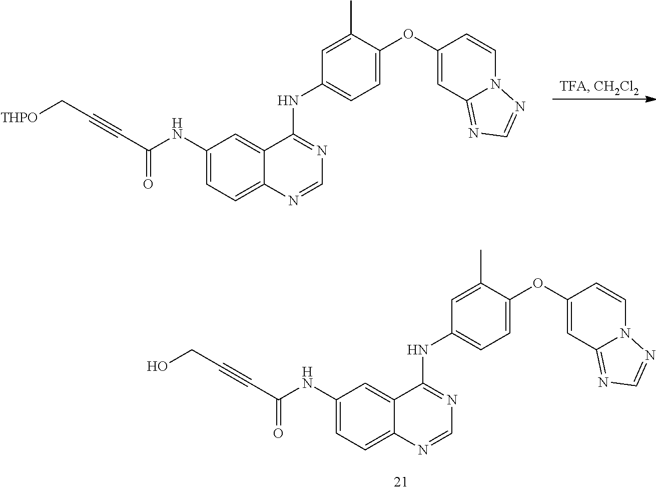

A mixture of N-[4-[(3-methyl-4-[[1,2,4]triazolo[1,5-a]pyridin-7-yloxy]phenyl)amino]quinazolin-6-yl]-4-(oxan-2-yloxy)but-2-ynamide (120.0 mg, 0.21 mmol) and TFA (3.0 mL) in CH₂Cl₂ (3.0 mL) was stirred at room temperature for 1 h. After the reaction was completed, the resulting mixture was concentrated under vacuum. The resulting mixture was diluted with CH₂Cl₂. The pH value of the mixture was adjusted to pH 7 with saturated NaHCO₃(aq.). The resulting mixture was concentrated under reduced pressure. The residue was purified by reverse phase flash column chromatography with ACN in water (5% to 100% gradient in 40 min) to afford N-(4-((4-([1,2,4]triazolo[1,5-a]pyridin-7-yloxy)-3-methylphenyl)amino)quinazolin-6-yl)-4-hydroxybut-2-ynamide (Compound 21) (17.0 mg, 16%) as a white solid. LCMS (ESI, m/z): [M+H]⁺=466.2. ¹H NMR (400 MHz, DMSO-d₆): δ 11.10 (s, 1H), 9.92 (s, 1H), 8.94 (d, J=7.6 Hz, 1H), 8.78 (s, 1H), 8.55 (s, 1H), 8.38 (s, 1H), 7.89-7.76 (m, 4H), 7.20 (d, J=8.4 Hz, 1H), 7.05-7.02 (m, 1H), 6.81-6.78 (m, 1H), 5.64-5.60 (m, 1H), 4.34 (s, 2H), 2.19 (s, 3H).

### Example S22. Synthesis of 5-hydroxy-N-[4-[(3-methyl-4-[[1,2,4]triazolo[1,5-a]pyridin-7-yloxy]phenyl)amino]quinazolin-6-yl]pent-2-ynamide (Compound 22)

### Step 1: Synthesis of 5-[(tert-butyldimethylsilyl)oxy]-N-[4-[(3-methyl-4-[[1,2,4]triazolo[1,5-a]pyridin-7-yloxy]phenyl)amino]quinazolin-6-yl]pent-2-ynamide

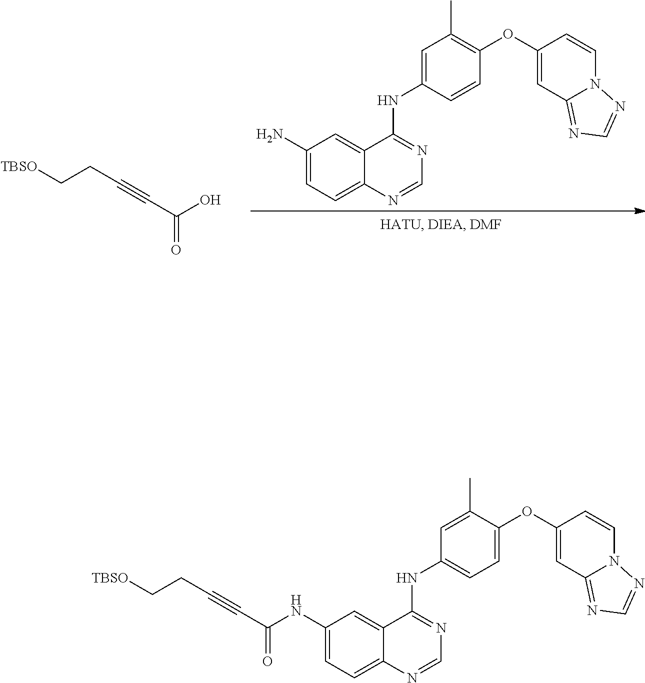

To a solution of 5-[(tert-butyldimethylsilyl)oxy]pent-2-ynoic acid (300.0 mg, 1.31 mmol) in DMF (15.0 mL) was added N4-(3-methyl-4-[[1,2,4]triazolo[1,5-a]pyridin-7-yloxy]phenyl)quinazoline-4,6-diamine (604.4 mg, 1.58 mmol), DIEA (1697.9 mg, 13.14 mmol) and HATU (1498.5 mg, 3.94 mmol). The mixture was stirred at room temperature for 1 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (93/7, v/v) to afford 5-[(tert-butyldimethylsilyl)oxy]-N-[4-[(3-methyl-4-[[1,2,4]triazolo[1,5-a]pyridin-7-yloxy]phenyl)amino]quinazolin-6-yl]pent-2-ynamide (350.0 mg, 45%) as a yellow solid. LCMS (ESI, m/z): [M+H]⁺=594.3.

### Step 2: Synthesis of 5-hydroxy-N-[4-[(3-methyl-4-[[1,2,4]triazolo[1,5-a]pyridin-7-yloxy]phenyl)amino]quinazolin-6-yl]pent-2-ynamide (Compound 22)

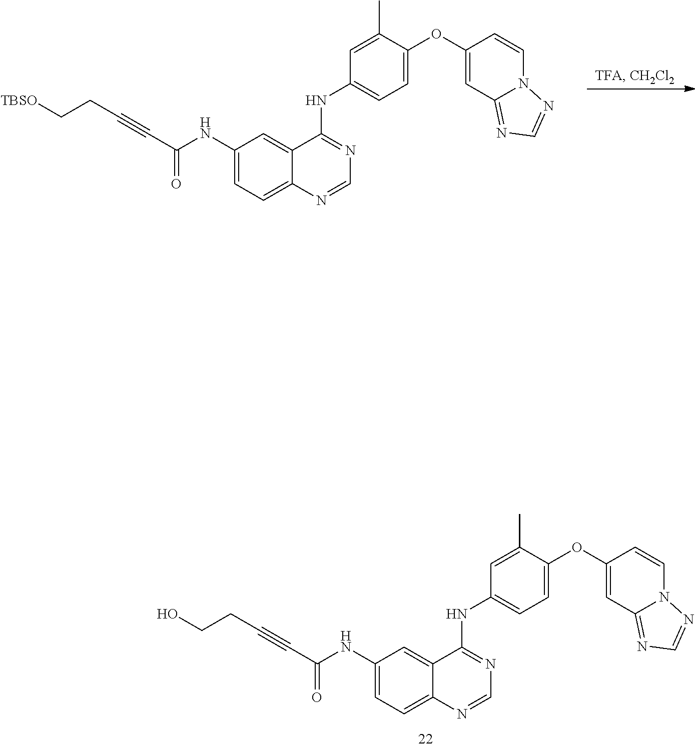

A mixture of 5-[(tert-butyldimethylsilyl)oxy]-N-[4-[(3-methyl-4-[[1,2,4]triazolo[1,5-a]pyridin-7-yloxy]phenyl)amino]quinazolin-6-yl]pent-2-ynamide (300.0 mg, 0.51 mmol) and TFA (5.0 mL) in CH₂Cl₂ (5.0 mL) was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (87/13, v/v) and then purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 12% B to 23% B in 7 min; 254/220 nm) to afford 5-hydroxy-N-[4-[(3-methyl-4-[[1,2,4]triazolo[1,5-a]pyridin-7-yloxy]phenyl)amino]quinazolin-6-yl]pent-2-ynamide (Compound 22) (11.8 mg, 5%). LCMS (ESI, m/z): [M+H]⁺=480.2. ¹H NMR (300 MHz, DMSO-d₆): δ 10.92 (s, 1H), 9.87 (s, 1H), 8.93 (d, J=7.5 Hz, 1H), 8.75 (s, 1H), 8.55 (s, 1H), 8.38 (s, 1H), 7.83-7.76 (m, 4H), 7.20 (d, J=8.7 Hz, 1H), 7.05-7.01 (m, 1H), 6.81 (d, J=2.4 Hz, 1H), 5.03 (s, 1H), 3.68-3.59 (m, 2H), 2.61-2.57 (m, 2H), 2.19 (s, 3H).

### Example S23. Synthesis of N-[4-[(2-fluoro-5-methyl-4-[[1,2,4]triazolo[1,5-a]pyridin-7-yloxy]phenyl)amino]quinazolin-6-yl]but-2-ynamide (Compound 23)

### Step 1: Synthesis of [1,2,4]triazolo[1,5-a]pyridin-7-ol

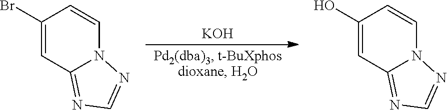 [SMILES: Brc1ccn2ncnc2c1 ; "KOH" ; "Pd2(dba)3, t-BuXphosdioxane, H2O" ; Oc1ccn2ncnc2c1]

To a solution of 7-bromo-[1,2,4]triazolo[1,5-a]pyridine (501.0 mg, 2.53 mmol) in 1,4-dioxane (8.0 mL)/H₂O (2.0 mL) was added KOH (625.7 mg, 11.15 mmol), Pd₂(dba)₃ (263.1 mg, 0.28 mmol) and t-BuXPhos (231.1 mg, 0.54 mmol) at room temperature. The resulting mixture was stirred at 100° C. for 4 h. After the reaction was completed, the mixture was cooled to room temperature and then extracted with EtOAc. The pH value of the aqueous layer was adjusted to pH 5 with 1N HCl. The mixture was evaporated in vacuo. The residue was purified by flash column chromatography with CH₂Cl₂/MeOH (5/1, v/v) to afford [1,2,4]triazolo[1,5-a]pyridin-7-ol (140.0 mg, 40%) as a white solid. LCMS (ESI, m/z): [M+H]⁺=136.0.

### Step 2: Synthesis of 7-(5-fluoro-2-methyl-4-nitrophenoxy)-[1,2,4]triazolo[1,5-a]pyridine

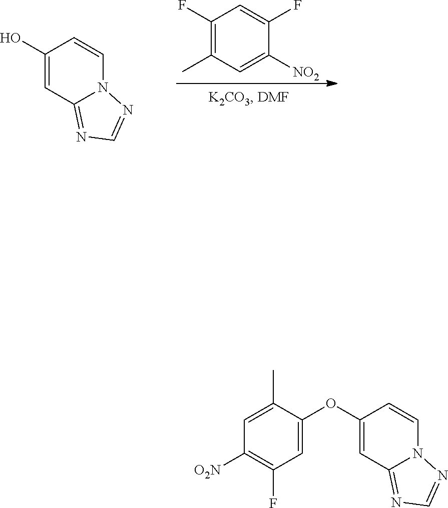

To a solution of [1,2,4]triazolo[1,5-a]pyridin-7-ol (1.3 g, 9.62 mmol) in DMF (30.0 mL) was added 1,5-difluoro-2-methyl-4-nitrobenzene (5.0 g, 28.86 mmol) and K₂CO₃ (8.0 g, 57.72 mmol) at room temperature. The resulting mixture was stirred at room temperature for 4 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (2/1, v/v) to afford 7-(5-fluoro-2-methyl-4-nitrophenoxy)-[1,2,4]triazolo[1,5-a]pyridine (1.1 g, 39%) as a white solid. LCMS (ESI, m/z): [M+H]⁺=289.1.

### Step 3: Synthesis of 2-fluoro-5-methyl-4-[[1,2,4]triazolo[1,5-a]pyridin-7-yloxy]aniline

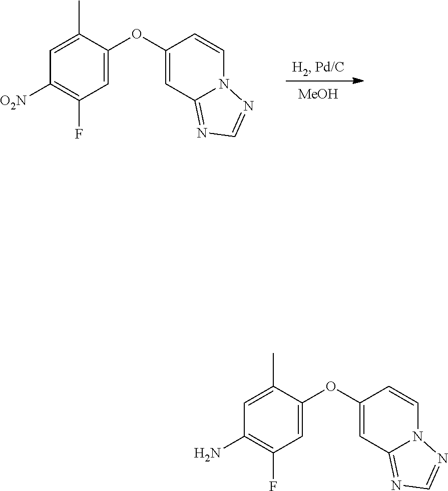 [SMILES: "MeOH" ; Cc1cc([N+](=O)[O-])c(F)cc1Oc1ccn2ncnc2c1 ; "H2, Pd/C" ; Cc1cc(N)c(F)cc1Oc1ccn2ncnc2c1]

To a solution of 7-(5-fluoro-2-methyl-4-nitrophenoxy)-[1,2,4]triazolo[1,5-a]pyridine (500.0 mg, 1.73 mmol) in MeOH (10.0 mL) was added Pd/C (150.0 mg, dry) at room temperature. The resulting mixture was stirred at room temperature for 2 h under H₂. After the reaction was completed, the resulting mixture was filtered. The filtrate was concentrated under reduced pressure to afford 2-fluoro-5-methyl-4-[[1,2,4]triazolo[1,5-a]pyridin-7-yloxy]aniline (380.0 mg, crude) as a white solid. LCMS (ESI, m/z): [M+H]⁺=259.1.

### Step 4: Synthesis of N-(2-fluoro-5-methyl-4-[[1,2,4]triazolo[1,5-a]pyridin-7-yloxy]phenyl)-6-nitroquinazolin-4-amine

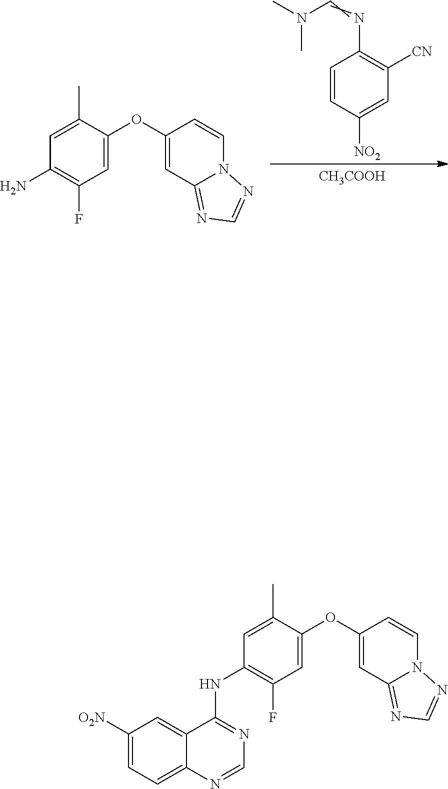

To a solution of 2-fluoro-5-methyl-4-[[1,2,4]triazolo[1,5-a]pyridin-7-yloxy]aniline (530.0 mg, 2.05 mmol) in CH₃COOH (10.0 mL) was added N′-(2-cyano-4-nitrophenyl)-N,N-dimethylformimidamide (492.6 mg, 2.26 mmol) at room temperature. The resulting mixture was stirred at 85° C. for 2 h. After the reaction was completed, the resulting mixture was concentrated under reduced pressure. The residue was dissolved with H₂O. The pH value of the aqueous layer was adjusted to pH 8 with NaHCO₃(aq.) and then filtered. The solid was collected and dried to afford N-(2-fluoro-5-methyl-4-[[1,2,4]triazolo[1,5-a]pyridin-7-yloxy]phenyl)-6-nitroquinazolin-4-amine (850.0 mg, crude) as a yellow solid. LCMS (ESI, m/z): [M+H]⁺=432.1.

### Step 5: Synthesis of N4-(2-fluoro-5-methyl-4-[[1,2,4]triazolo[1,5-a]pyridin-7-yloxy]phenyl)quinazoline-4,6-diamine

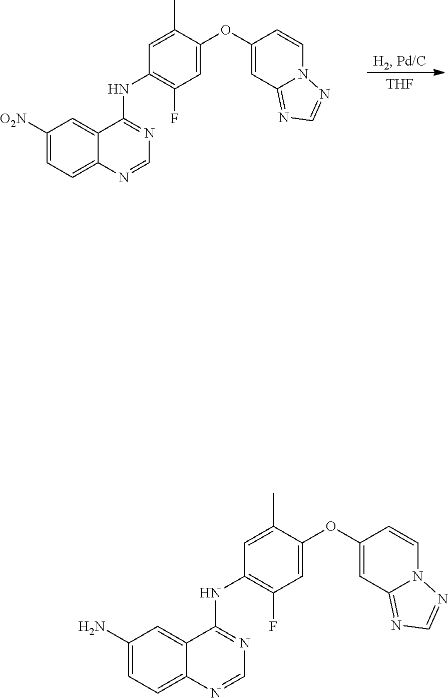 [SMILES: Cc1cc(Nc2ncnc3ccc([N+](=O)[O-])cc23)c(F)cc1Oc1ccn2ncnc2c1 ; "H2, Pd/C" ; Cc1cc(Nc2ncnc3ccc(N)cc23)c(F)cc1Oc1ccn2ncnc2c1 ; "THF"]

To a solution of N-(2-fluoro-5-methyl-4-[[1,2,4]triazolo[1,5-a]pyridin-7-yloxy]phenyl)-6-nitroquinazolin-4-amine (380.0 mg, 0.88 mmol) in THF (20.0 mL) was added Pd/C (114.0 mg, dry) at room temperature. The resulting mixture was stirred at room temperature for 2 h under H₂. After the reaction was completed, the resulting mixture was filtered. The filtrate was concentrated under reduced pressure to afford N4-(2-fluoro-5-methyl-4-[[1,2,4]triazolo[1,5-a]pyridin-7-yloxy]phenyl)quinazoline-4,6-diamine (280.0 mg, crude) as a white solid. LCMS (ESI, m/z): [M+H]⁺=402.1.

### Step 6: Synthesis of N-[4-[(2-fluoro-5-methyl-4-[[1,2,4]triazolo[1,5-a]pyridin-7-yloxy]phenyl)amino]quinazolin-6-yl]but-2-ynamide (Compound 23)

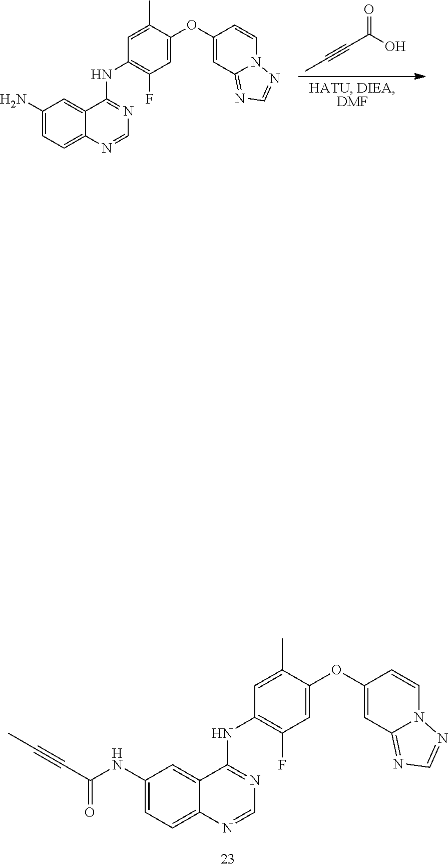

To a solution of N4-(2-fluoro-5-methyl-4-[[1,2,4]triazolo[1,5-a]pyridin-7-yloxy]phenyl)quinazoline-4,6-diamine (280.0 mg, 0.70 mmol) in DMF (3.0 mL) was added 2-butynoic acid (117.3 mg, 1.39 mmol), DIEA (540.9 mg, 4.19 mmol) and HATU (663.1 mg, 1.74 mmol) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Shield RP18 OBD Column, 19×250 mm, 10 um; Mobile Phase A: Water (0.05% FA), Mobile Phase B: MeOH-Preparative; Flow rate: 25 mL/min; Gradient: 41% B to 51% B in 9 min; 254 nm) to afford N-[4-[(2-fluoro-5-methyl-4-[[1,2,4]triazolo[1,5-a]pyridin-7-yloxy]phenyl)amino]quinazolin-6-yl]but-2-ynamide (Compound 23) (2.1 mg, 1%) as a light yellow solid. LCMS (ESI, m/z): [M+H^{]}+=468.2.41 NMR (300 MHz, DMSO-d₆): δ 10.96 (s, 1H), 9.86 (s, 1H), 8.97 (d, J=7.5 Hz, 1H), 8.75 (s, 1H), 8.46-8.42 (m, 2H), 7.82-7.79 (m, 2H), 7.54 (d, J=8.7 Hz, 1H), 7.29-7.25 (m, 1H), 7.08-7.05 (m, 1H), 6.95 (d, J=2.4 Hz, 1H), 2.18 (s, 3H), 2.09 (s, 3H).

### Example S24. Synthesis of N-(4-((4-([1,2,4]triazolo[1,5-a]pyridin-7-yloxy)-2-fluoro-3-methylphenyl)amino)quinazolin-6-yl)but-2-ynamide (Compound 24)

### Step 1: Synthesis of 7-(3-fluoro-2-methyl-4-nitrophenoxy)-[1,2,4]triazolo[1,5-a]pyridine

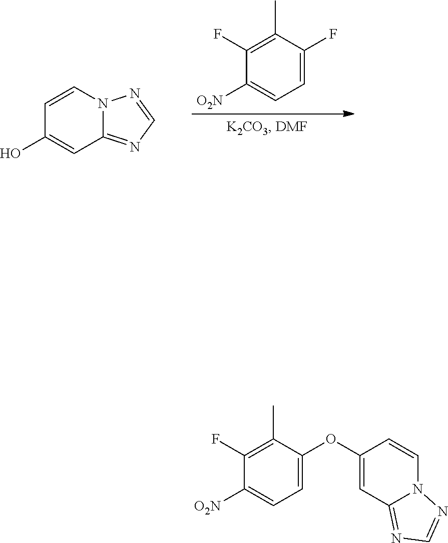

To a solution of [1,2,4]triazolo[1,5-a]pyridin-7-ol (2.0 g, 14.80 mmol) in DMF (20.0 mL) was added 1,3-difluoro-2-methyl-4-nitrobenzene (5.1 g, 29.60 mmol) and K₂CO₃ (12.8.0 g, 88.78 mmol) at room temperature. The resulting mixture was stirred at room temperature for 4 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (2/1, v/v) to afford 7-(3-fluoro-2-methyl-4-nitrophenoxy)-[1,2,4]triazolo[1,5-a]pyridine (700.0 mg, 16%) as a white solid. LCMS (ESI, m/z): [M+H]⁺=289.1.

### Step 2: Synthesis of 4-([1,2,4]triazolo[1,5-a]pyridin-7-yloxy)-2-fluoro-3-methylaniline

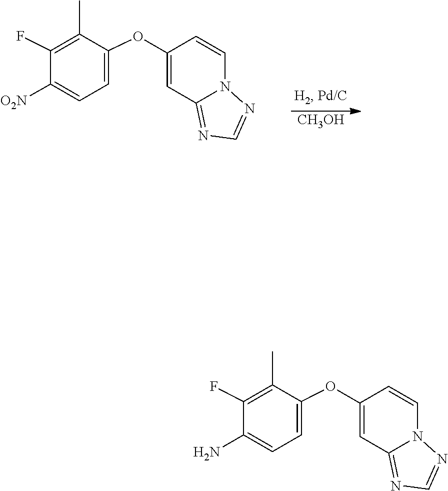

To a solution of 7-(3-fluoro-2-methyl-4-nitrophenoxy)-[1,2,4]triazolo[1,5-a]pyridine (700.0 mg, 2.42 mmol) in MeOH (10.0 mL) was added Pd/C (129.2 mg, dry) at room temperature under N₂. The resulting mixture was stirred at room temperature for 2 h under H₂. After the reaction was completed, the resulting mixture was filtered. The filtrate was concentrated under reduced pressure to afford 4-([1,2,4]triazolo[1,5-a]pyridin-7-yloxy)-2-fluoro-3-methylaniline (380.0 mg, crude) as a white solid. LCMS (ESI, m/z): [M+H]⁺=259.1.

### Step 3: Synthesis of N-(4-([1,2,4]triazolo[1,5-a]pyridin-7-yloxy)-2-fluoro-3-methylphenyl)-6-nitroquinazolin-4-amine

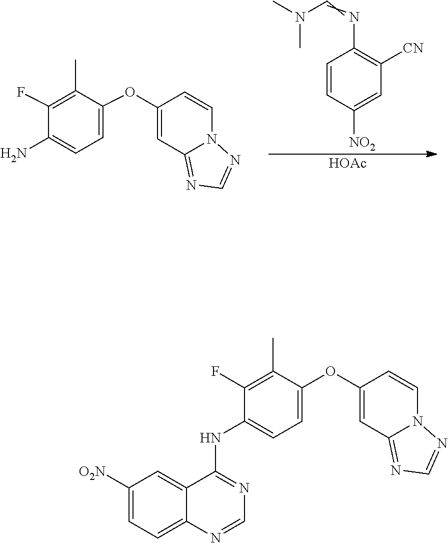 [SMILES: Cc1c(Oc2ccn3ncnc3c2)ccc(N)c1F ; CN(C)C=Nc1ccc([N+](=O)[O-])cc1C#N ; Cc1c(Oc2ccn3ncnc3c2)ccc(Nc2ncnc3ccc([N+](=O)[O-])cc23)c1F ; "HOAc"]

To a solution of 4-([1,2,4]triazolo[1,5-a]pyridin-7-yloxy)-2-fluoro-3-methylaniline (380.0 mg, 1.47 mmol) in CH₃COOH (10.0 mL) was added N′-(2-cyano-4-nitrophenyl)-N,N-dimethylformimidamide (353.2 mg, 1.62 mmol) at room temperature. The resulting mixture was stirred at 85° C. for 2 h. After the reaction was completed, the resulting mixture was concentrated under reduced pressure. The pH value of the residue was adjusted to pH 8 with NaHCO₃(aq.) and then filtered. The solid was collected and dried to afford N-(4-([1,2,4]triazolo[1,5-a]pyridin-7-yloxy)-2-fluoro-3-methylphenyl)-6-nitroquinazolin-4-amine (400.0 mg, crude) as a yellow solid. LCMS (ESI, m/z): [M+H]⁺=432.1.

### Step 4: Synthesis of N4-(4-([1,2,4]triazolo[1,5-a]pyridin-7-yloxy)-2-fluoro-3-methylphenyl)quinazoline-4,6-diamine

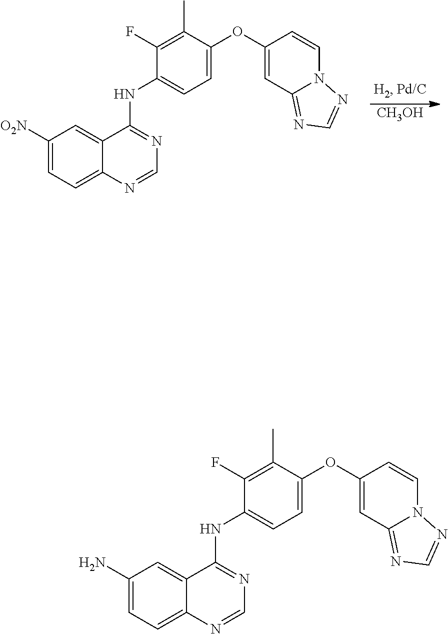

To a solution of N-(4-([1,2,4]triazolo[1,5-a]pyridin-7-yloxy)-2-fluoro-3-methylphenyl)-6-nitroquinazolin-4-amine (380.0 mg, 0.88 mmol) in CH₃OH (20.0 mL) was added Pd/C (46.9 mg, dry) at room temperature. The resulting mixture was stirred at room temperature for 2 h under H₂. After the reaction was completed, the resulting mixture was filtered. The filtrate was concentrated under reduced pressure to afford N4-(4-([1,2,4]triazolo[1,5-a]pyridin-7-yloxy)-2-fluoro-3-methylphenyl)quinazoline-4,6-diamine (200.0 mg, crude) as a white solid. LCMS (ESI, m/z): [M+H]⁺=402.1.

### Step 5: Synthesis of N-(4-((4-([1,2,4]triazolo[1,5-a]pyridin-7-yloxy)-2-fluoro-3-methylphenyl)amino)quinazolin-6-yl)but-2-ynamide (Compound 24)

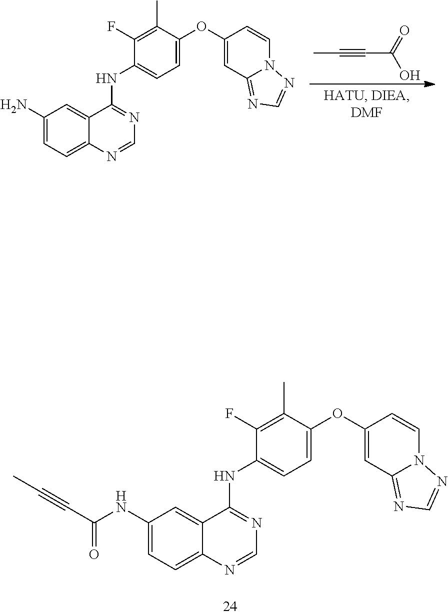

To a solution of N4-(4-([1,2,4]triazolo[1,5-a]pyridin-7-yloxy)-2-fluoro-3-methylphenyl)quinazoline-4,6-diamine (120.0 mg, 0.30 mmol) in DMF (5.0 mL) was added 2-butynoic acid (25.1 mg, 0.30 mmol), DIEA (46.4 mg, 0.36 mmol) and HATU (136.4 mg, 0.36 mmol) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Shield RP18 OBD Column, 19×250 mm, 10 um; Mobile Phase A: Water (10 mmol/L NH₄HCO₃), Mobile Phase B: ACN-Preparative; Flow rate: 25 mL/min; Gradient: 32% B to 38% B in 8 min; 254 nm) to afford N-(4-((4-([1,2,4]triazolo[1,5-a]pyridin-7-yloxy)-2-fluoro-3-methylphenyl)amino)quinazolin-6-yl)but-2-ynamide (Compound 24) (2.4 mg, 1%) as a light yellow solid. LCMS (ESI, m/z): [M+H]⁺=468.2. ¹H NMR (300 MHz, CD₃OD): δ 8.78 (d, J=7.5 Hz, 1H), 8.69 (s, 1H), 8.45 (s, 1H), 8.32 (s, 1H), 7.89-7.79 (m, 2H), 7.62-7.53 (m, 1H), 7.14-7.08 (m, 2H), 7.02 (d, J=2.4 Hz, 1H), 2.22 (s, 3H), 2.09 (s, 3H).

### Example S25. Synthesis of 5-hydroxy-N-[4-[(4-[[1,2,4]triazolo[1,5-a]pyridin-7-ylmethyl]phenyl)amino]quinazolin-6-yl]pent-2-ynamide (Compound 25)

### Step 1: Synthesis of 5-[(tert-butyldimethylsilyl)oxy]-N-[4-[(4-[[1,2,4]triazolo[1,5-a]pyridin-7-ylmethyl]phenyl)amino]quinazolin-6-yl]pent-2-ynamide

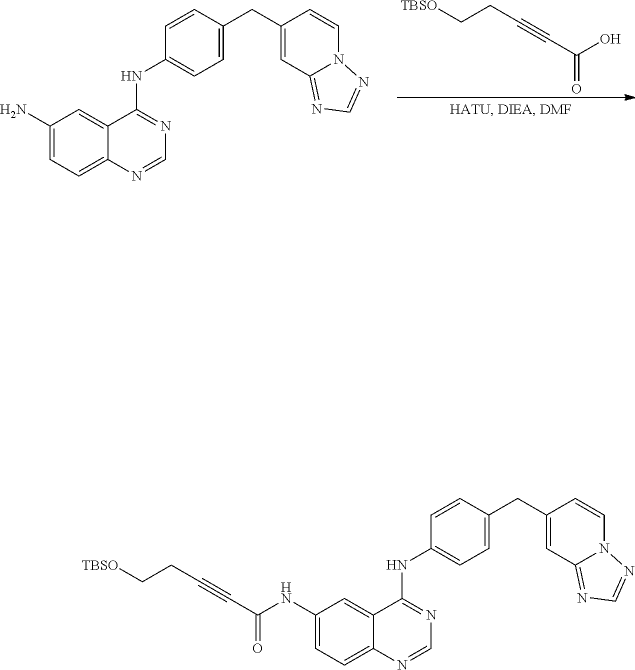 [SMILES: Nc1ccc2ncnc(Nc3ccc(Cc4ccn5ncnc5c4)cc3)c2c1 ; CC(C)(C)[Si](C)(C)OCCC#CC(=O)O ; CC(C)(C)[Si](C)(C)OCCC#CC(=O)Nc1ccc2ncnc(Nc3ccc(Cc4ccn5ncnc5c4)cc3)c2c1]

To a solution of N4-(4-[[1,2,4]triazolo[1,5-a]pyridin-7-ylmethyl]phenyl)quinazoline-4,6-diamine (340.0 mg, 0.93 mmol) in DMF (10.0 mL) was added DIEA (505.6 mg, 3.91 mmol), 5-[(tert-butyldimethylsilyl)oxy]pent-2-ynoic acid (422.6 mg, 1.85 mmol) and HATU (703.7 mg, 1.85 mmol) at 0° C. under N₂. The resulting mixture was stirred at 0° C. for 1 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The mixture was purified by flash column chromatography with MeOH/DCM (5/95, v/v) to afford 5-[(tert-butyldimethylsilyl)oxy]-N-[4-[(4-[[1,2,4]triazolo[1,5-a]pyridin-7-ylmethyl]phenyl)amino]quinazolin-6-yl]pent-2-ynamide (130.0 mg, 24%) as a light yellow solid. LCMS (ESI, m/z): [M+H]⁺=578.2.

### Step 2: Synthesis of 5-hydroxy-N-[4-[(4-[[1,2,4]triazolo[1,5-a]pyridin-7-ylmethyl]phenyl)amino]quinazolin-6-yl]pent-2-ynamide (Compound 25)

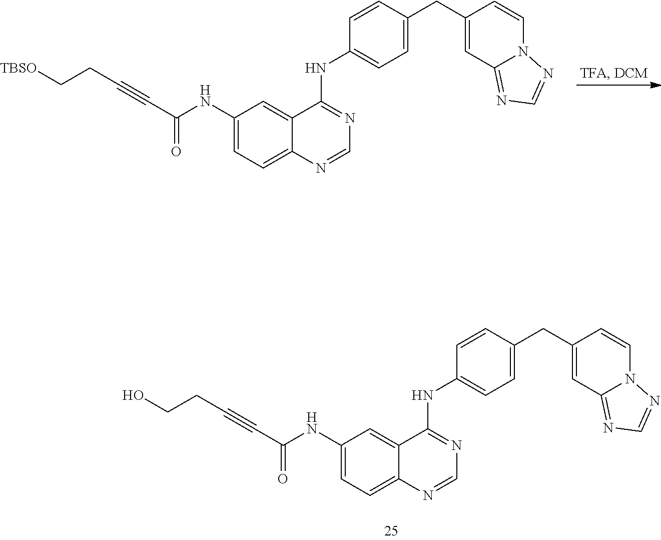 [SMILES: CC(C)(C)[Si](C)(C)OCCC#CC(=O)Nc1ccc2ncnc(Nc3ccc(Cc4ccn5ncnc5c4)cc3)c2c1 ; O=C(C#CCCO)Nc1ccc2ncnc(Nc3ccc(Cc4ccn5ncnc5c4)cc3)c2c1]

To a solution of 5-[(tert-butyldimethylsilyl)oxy]-N-[4-[(4-[[1,2,4]triazolo[1,5-a]pyridin-7-ylmethyl]phenyl)amino]quinazolin-6-yl]pent-2-ynamide (130.0 mg, 0.22 mmol) in DCM (5.0 mL) was added TFA (2.0 mL). The mixture was stirred at room temperature for 1 h. After the reaction was completed, the mixture was concentrated under reduced pressure. The residue was purified by flash column chromatography with MeOH/DCM (1/10, v/v) and then purified by Prep-HPLC with the following conditions: Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 28% B to 34% B in 9 min; 254/220 nm; RT1:8.62 min to afford 5-hydroxy-N-[4-[(4-[[1,2,4]triazolo[1,5-a]pyridin-7-ylmethyl]phenyl)amino]quinazolin-6-yl]pent-2-ynamide (Compound 25) (25.8 mg, 24%) as a white solid. LCMS (ESI, m/z): [M+H^{]}+=464.1. ¹H NMR (300 MHz, DMSO-d₆): δ 10.88 (s, 1H), 9.78 (s, 1H), 8.84 (d, J=7.2 Hz, 1H), 8.68 (s, 1H), 8.47-8.42 (m, 2H), 7.82-7.70 (m, 5H), 7.32 (d, J=8.4 Hz, 2H), 7.08-7.05 (m, 1H), 5.02-4.98 (m, 1H), 4.08 (s, 2H), 3.64-3.57 (m, 2H), 2.59-2.55 (m, 2H).

### Example S26. Synthesis of N-[5-fluoro-4-[(3-methyl-4-[[1,2,4]triazolo[1,5-a]pyridin-7-yloxy]phenyl)amino]quinazolin-6-yl]but-2-ynamide (Compound 26)

### Step 1: Synthesis of (E)-N′-(4-bromo-2-cyano-3-fluorophenyl)-N,N-dimethylmethanimidamide

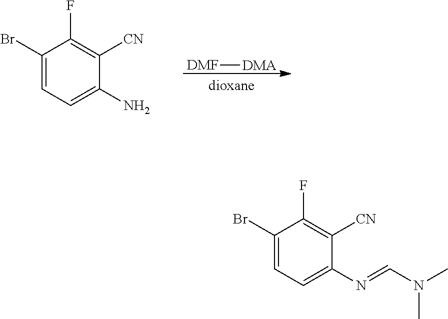

To a solution of 6-amino-3-bromo-2-fluorobenzonitrile (500.0 mg, 2.33 mmol) in 1,4-dioxane (5.0 mL) was added DMF-DMA (554.2 mg, 4.65 mmol) at room temperature. The resulting mixture was stirred at 75° C. for 30 min. After the reaction was completed, the resulting mixture was concentrated under reduced pressure to afford (E)-N′-(4-bromo-2-cyano-3-fluorophenyl)-N,N-dimethylmethanimidamide (600.0 mg, crude) as a yellow solid. LCMS (ESI, m/z): [M+H]⁺=270.0.

### Step 2: Synthesis of 6-bromo-5-fluoro-N-(3-methyl-4-[[1,2,4]triazolo[1,5-a]pyridin-7-yloxy]phenyl)quinazolin-4-amine

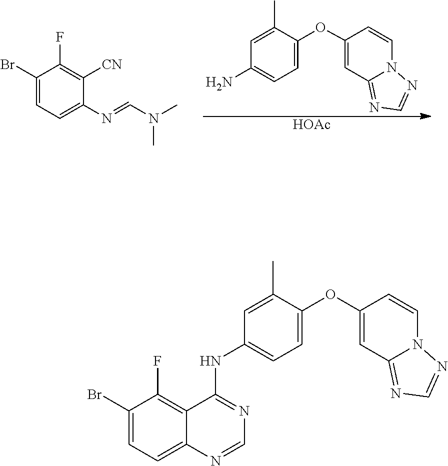 [SMILES: CN(C)/C=N/c1ccc(Br)c(F)c1C#N ; Cc1cc(N)ccc1Oc1ccn2ncnc2c1 ; Cc1cc(Nc2ncnc3ccc(Br)c(F)c23)ccc1Oc1ccn2ncnc2c1 ; "HOAc"]

To a solution of (E)-N′-(4-bromo-2-cyano-3-fluorophenyl)-N,N-dimethylmethanimidamide (300.0 mg, 1.11 mmol) in acetic acid (10.0 mL) was added 3-methyl-4-[[1,2,4]triazolo[1,5-a]pyridin-7-yloxy]aniline (266.9 mg, 1.11 mmol) at room temperature. The resulting mixture was stirred at 85° C. for 2 h. After the reaction was completed, the resulting mixture was concentrated under reduced pressure. The residue was purified by flash column chromatography with CH₂Cl₂/CH₃OH (89/11, v/v) to afford 6-bromo-5-fluoro-N-(3-methyl-4-[[1,2,4]triazolo[1,5-a]pyridin-7-yloxy]phenyl)quinazolin-4-amine (270.0 mg, 52%) as a yellow solid. LCMS (ESI, m/z): [M+H]⁺=465.0.

### Step 3: Synthesis of N6-(diphenylmethylidene)-5-fluoro-N4-(3-methyl-4-[[1,2,4]triazolo[1,5-a]pyridin-7-yloxy]phenyl)quinazoline-4,6-diamine

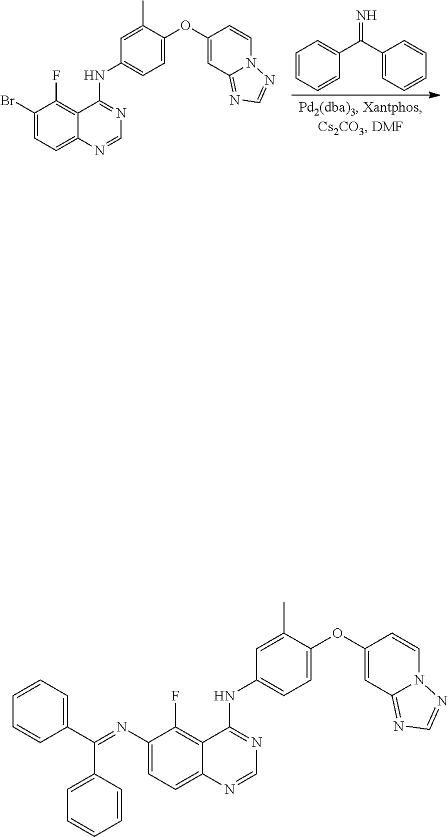

To a solution of diphenylmethanimine (3.6 g, 19.97 mmol) in DMF (40.0 mL) was added 6-bromo-5-fluoro-N-(3-methyl-4-[[1,2,4]triazolo[1,5-a]pyridin-7-yloxy]phenyl)quinazolin-4-amine (3.1 g, 1.40 mmol), Cs₂CO₃ (4.3 g, 13.32 mmol), Xantphos (1.5 g, 2.66 mmol) and Pd₂(dba)₃ (1.2 g, 1.33 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/EtOAc (67/33, v/v) (0/100, v/v) to afford N6-(diphenylmethylidene)-5-fluoro-N4-(3-methyl-4-[[1,2,4]triazolo[1,5-a]pyridin-7-yloxy]phenyl)quinazoline-4,6-diamine (2.2 g, 58%) as a yellow solid. LCMS (ESI, m/z): [M+H]⁺=566.2.

### Step 4: Synthesis of 5-fluoro-N4-(3-methyl-4-[[1,2,4]triazolo[1,5-a]pyridin-7-yloxy]phenyl)quinazoline-4,6-diamine

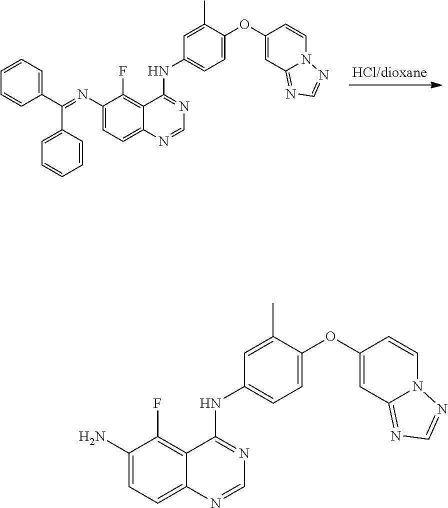 [SMILES: Cc1cc(Nc2ncnc3ccc(N=C(c4ccccc4)c4ccccc4)c(F)c23)ccc1Oc1ccn2ncnc2c1 ; Cc1cc(Nc2ncnc3ccc(N)c(F)c23)ccc1Oc1ccn2ncnc2c1]

The solution of N6-(diphenylmethylidene)-5-fluoro-N4-(3-methyl-4-[[1,2,4]triazolo[1,5-a]pyridin-7-yloxy]phenyl)quinazoline-4,6-diamine (2.2 g, 3.89 mmol) in HCl/1,4-dioxane (30.0 mL, 4 mol/L) was stirred at room temperature for 2 h. After the reaction was completed, the resulting mixture was diluted with water. The pH value of the mixture was adjusted to pH 8 with NaHCO₃(aq.). The resulting mixture was extracted with EtOAc. The combined organic layer was washed with brine, dried over anhydrous Na₂SO₄ and filtered. The filtrate was concentrated under vacuum. The mixture was purified by flash column chromatography with petroleum ether/EtOAc (2/1, v/v) to afford 5-fluoro-N4-(3-methyl-4-[[1,2,4]triazolo[1,5-a]pyridin-7-yloxy]phenyl)quinazoline-4,6-diamine (930.0 mg, 59%) as a light yellow solid. LCMS (ESI, m/z): [M+H]⁺=402.1.

### Step 5: Synthesis of N-[5-fluoro-4-[(3-methyl-4-[[1,2,4]triazolo[1,5-a]pyridin-7-yloxy]phenyl)amino]quinazolin-6-yl]but-2-ynamide (Compound 26)

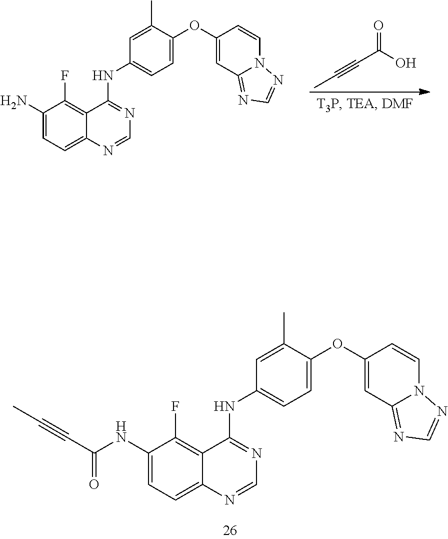

To a solution of 5-fluoro-N4-(3-methyl-4-[[1,2,4]triazolo[1,5-a]pyridin-7-yloxy]phenyl)quinazoline-4,6-diamine (300.0 mg, 0.75 mmol) in DMF (20.0 mL) was added 2-butynoic acid (62.8 mg, 0.75 mmol), TEA (151.3 mg, 1.50 mmol) and T₃P (951.2 mg, 50% in EtOAc). The mixture was stirred at room temperature for 16 h. After the reaction was completed, the resulting mixture was diluted with H₂O and extracted with EtOAc. The combined organic layer was washed with brine, dried over anhydrous sodium sulfate and filtered. The filtrate was concentrated under reduced pressure. The residue was purified by Prep-HPLC with the following conditions: Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 34% B to 44% B in 8 min; 254/220 nm; RT1:7.02 min to afford N-[5-fluoro-4-[(3-methyl-4-[[1,2,4]triazolo[1,5-a]pyridin-7-yloxy]phenyl)amino]quinazolin-6-yl]but-2-ynamide (Compound 26) (16.9 mg, 5%) as a white solid. LCMS (ESI, m/z): [M+H]⁺=468.0. ¹H NMR (300 MHz, DMSO-d₆): δ 10.72 (s, 1H), 9.19 (s, 1H), 8.96-8.94 (m, 1H), 8.58 (s, 1H), 8.39 (s, 1H), 8.04-7.98 (m, 1H), 7.77-7.63 (m, 3H), 7.23 (d, J=8.7 Hz, 1H), 7.06-7.02 (m, 1H), 6.81 (d, J=2.1 Hz, 1H), 2.20 (s, 3H), 2.09 (s, 3H).

### Example S27. Synthesis of N-[4-[(3-methyl-4-[[1,2,4]triazolo[1,5-a]pyridin-7-ylmethyl]phenyl)amino]quinazolin-6-yl]but-2-ynamide (Compound 27)

### Step 1: Synthesis of (2-methyl-4-nitrophenyl)methanol

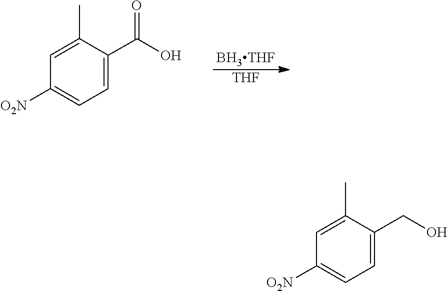

To a solution of 2-methyl-4-nitrobenzoic acid (10.0 g, 55.81 mmol) in THF (100.0 mL) was added BH₃-THF (110 mL, 1 mol/L) at 0° C. under N₂. The resulting mixture was stirred at room temperature for 30 min and then stirred at 75° C. for 5 h. After the reaction was completed, the reaction was quenched with water and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (56/34, v/v) to afford (2-methyl-4-nitrophenyl)methanol (5.0 g, 53%) as a yellow solid.

### Step 2: Synthesis of 1-(bromomethyl)-2-methyl-4-nitrobenzene

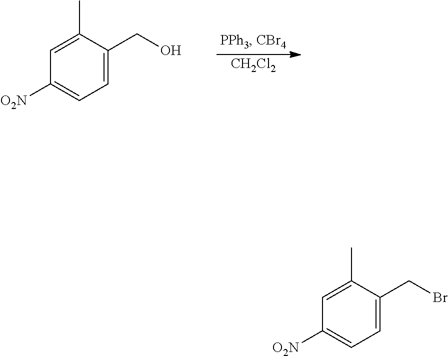

To a solution of (2-methyl-4-nitrophenyl)methanol (9.0 g, 53.84 mmol) in CH₂Cl₂ (100.0 mL) was added PPh₃ (21.2 g, 80.76 mmol) and CBr₄ (26.8 g, 80.76 mmol) at 0° C. The resulting mixture was stirred at room temperature for 3 h. After the reaction was completed, the resulting mixture was concentrated under reduced pressure. The residue was purified by flash column chromatography with petroleum ether/ethyl acetate (87/13, v/v) to afford 1-(bromomethyl)-2-methyl-4-nitrobenzene (6.0 g, 48%) as a yellow solid.

### Step 3: Synthesis of 7-[(2-methyl-4-nitrophenyl)methyl]-[1,2,4]triazolo[1,5-a]pyridine

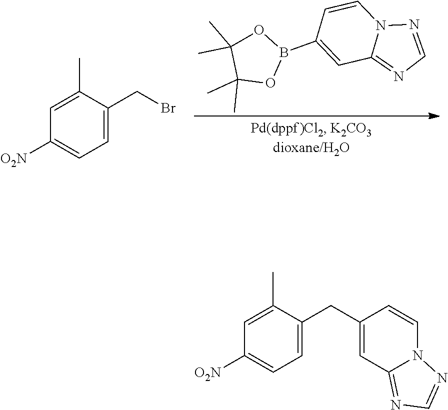

To a solution of 1-(bromomethyl)-2-methyl-4-nitrobenzene (1.0 g, 4.35 mmol) in 1,4-dioxane/H₂O (10.0/2.0 mL) was added 7-(4,4,5,5-tetramethyl-1,3,2-dioxaborolan-2-yl)-[1,2,4]triazolo[1,5-a]pyridine (3.2 g, 13.04 mmol), K₂CO₃ (1.8 g, 13.04 mmol) and Pd(dppf)Cl₂ (318.1 mg, 0.44 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (9/1, v/v) to afford 7-[(2-methyl-4-nitrophenyl)methyl]-[1,2,4]triazolo[1,5-a]pyridine (500.0 mg, 42%) as a yellow solid. LCMS (ESI, m/z): [M+H]⁺=269.1.

### Step 4: Synthesis of 3-methyl-4-[[1,2,4]triazolo[1,5-a]pyridin-7-ylmethyl]aniline

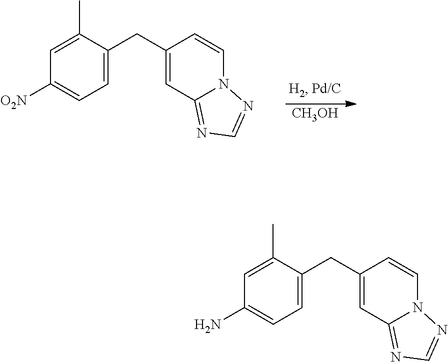

To a solution of 7-[(2-methyl-4-nitrophenyl)methyl]-[1,2,4]triazolo[1,5-a]pyridine (500.0 mg, 1.86 mmol) in CH₃OH (10.0 mL) was added Pd/C (198.3 mg, dry) at room temperature. The resulting mixture was stirred at room temperature for 2 h under H₂. After the reaction was completed, the resulting mixture was filtered. The filtrate was concentrated under reduced pressure. The residue was purified by flash column chromatography with CH₂Cl₂/CH₃OH (9/1, v/v) to afford 3-methyl-4-[[1,2,4]triazolo[1,5-a]pyridin-7-ylmethyl]aniline (400.0 mg, 90%) as a yellow solid. LCMS (ESI, m/z): [M+H]⁺=239.1.

### Step 5: Synthesis of N-(3-methyl-4-[[1,2,4]triazolo[1,5-a]pyridin-7-ylmethyl]phenyl)-6-nitroquinazolin-4-amine

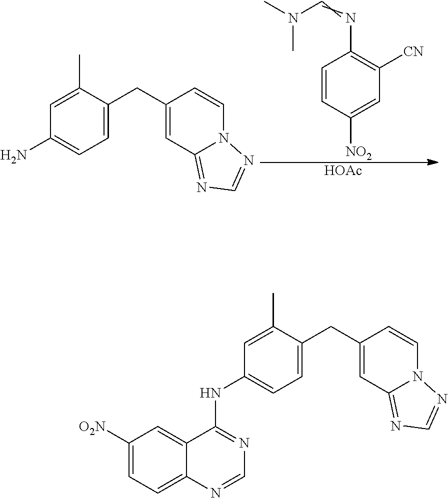 [SMILES: Cc1cc(N)ccc1Cc1ccn2ncnc2c1 ; CN(C)C=Nc1ccc([N+](=O)[O-])cc1C#N ; Cc1cc(Nc2ncnc3ccc([N+](=O)[O-])cc23)ccc1Cc1ccn2ncnc2c1 ; "HOAc"]

To a solution of 3-methyl-4-[[1,2,4]triazolo[1,5-a]pyridin-7-ylmethyl]aniline (400.0 mg, 1.68 mmol) in HOAc (5.0 mL) was added N′-(2-cyano-4-nitrophenyl)-N,N-dimethylformimidamide (366.3 mg, 1.68 mmol) at room temperature. The resulting mixture was stirred at 85° C. for 2 h. After the reaction was completed, the resulting mixture was concentrated under reduced pressure. The residue was purified by flash column chromatography with CH₂Cl₂/CH₃OH (86/14, v/v) to afford N-(3-methyl-4-[[1,2,4]triazolo[1,5-a]pyridin-7-ylmethyl]phenyl)-6-nitroquinazolin-4-amine (600.0 mg, 86%) as a yellow solid. LCMS (ESI, m/z): [M+H]⁺=412.1.

### Step 6: Synthesis of N4-(3-methyl-4-[[1,2,4]triazolo[1,5-a]pyridin-7-ylmethyl]phenyl)quinazoline-4,6-diamine

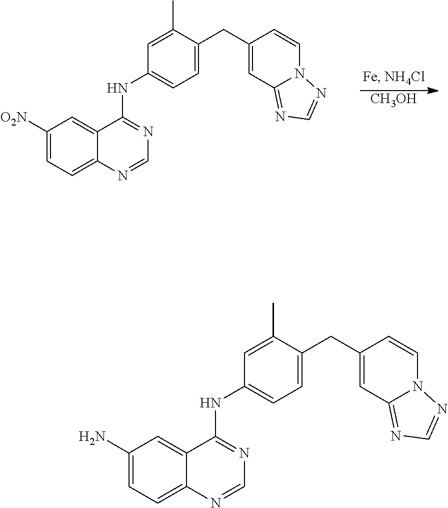

To a solution of N-(3-methyl-4-[[1,2,4]triazolo[1,5-a]pyridin-7-ylmethyl]phenyl)-6-nitroquinazolin-4-amine (600.0 mg, 1.46 mmol) in CH₃OH/H₂O (10.0/2.0 mL) added NH₄Cl (780.1 mg, 14.58 mmol) and Fe (814.4 mg, 14.58 mmol) at room temperature. The resulting mixture was stirred at 80° C. for 1 h. After the reaction was completed, the resulting mixture was filtered. The filtrate was concentrated under reduced pressure. The residue was purified by flash column chromatography with CH₂Cl₂/CH₃OH (9/1, v/v) to afford N4-(3-methyl-4-[[1,2,4]triazolo[1,5-a]pyridin-7-ylmethyl]phenyl)quinazoline-4,6-diamine (300.0 mg, 53%) as a yellow solid. LCMS (ESI, m/z): [M+H]⁺=382.1.

### Step 7: Synthesis of N-[4-[(3-methyl-4-[[1,2,4]triazolo[1,5-a]pyridin-7-ylmethyl]phenyl)amino]quinazolin-6-yl]but-2-ynamide (Compound 27)

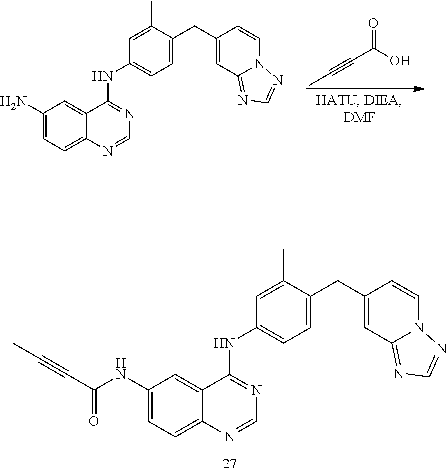

To a solution of N4-(3-methyl-4-[[1,2,4]triazolo[1,5-a]pyridin-7-ylmethyl]phenyl)quinazoline-4,6-diamine (300.0 mg, 0.78 mmol) in DMF (5.0 mL) was added 2-butynoic acid (85.9 mg, 1.02 mmol), DIEA (1.0 g, 7.86 mmol) and HATU (478.4 mg, 1.26 mmol) at room temperature. The resulting mixture was stirred at room temperature for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse flash column chromatography with CH₂Cl₂/CH₃OH (89/11, v/v) and then purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 11% B to 32% B in 7 min; 254 nm) to afford N-[4-[(3-methyl-4-[[1,2,4]triazolo[1,5-a]pyridin-7-ylmethyl]phenyl)amino]quinazolin-6-yl]but-2-ynamide (Compound 27) (1.0 mg, 0.3%) as a white solid. LCMS (ESI, m/z): [M+H]⁺=448.1.41 NMR (300 MHz, CD₃OD): δ 8.71 (d, J=6.9 Hz, 1H), 8.65 (d, J=2.1 Hz, 1H), 8.48 (s, 1H), 8.37 (s, 1H), 7.86-7.76 (m, 2H), 7.64-7.62 (m, 2H), 7.49 (s, 1H), 7.31 (J=8.7 Hz, 1H), 7.14-7.11 (m, 1H), 4.24 (s, 2H), 2.33 (s, 3H), 2.12 (s, 3H).

### Example S28. Synthesis of N-[5-fluoro-4-[(3-methyl-4-[[1,2,4]triazolo[1,5-a]pyridin-7-yloxy]phenyl)amino]quinazolin-6-yl]-N-methylbut-2-ynamide (Compound 28)

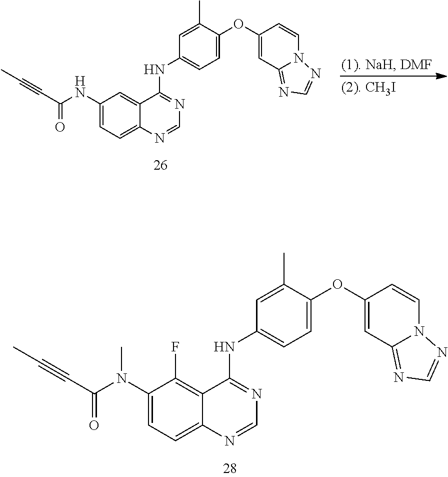 [SMILES: CC#CC(=O)Nc1ccc2ncnc(Nc3ccc(Oc4ccn5ncnc5c4)c(C)c3)c2c1 ; CC#CC(=O)N(C)c1ccc2ncnc(Nc3ccc(Oc4ccn5ncnc5c4)c(C)c3)c2c1F]

To a solution of N-[5-fluoro-4-[(3-methyl-4-[[1,2,4]triazolo[1,5-a]pyridin-7-yloxy]phenyl)amino]quinazolin-6-yl]but-2-ynamide (Compound 26) (320.0 mg, 0.68 mmol) in DMF (8.0 mL) was added NaH (82.1 mg, 60%) at 0° C. under N₂. The mixture was stirred at 0° C. for 30 min. Then CH₃I (97.1 mg, 0.68 mmol) was added to the mixture at 0° C. under N2. The reaction mixture was stirred at room temperature for 1 h. After the reaction was completed, the resulting mixture was diluted with H₂O and extracted with DCM. The combined organic layer was washed with brine, dried over anhydrous sodium sulfate and filtered. The filtrate was concentrated under reduced pressure. The residue was purified by Prep-HPLC with the following conditions: Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 36% B to 46% B in 7 min; 254/220 nm; RT1:6.72 min to afford N-[5-fluoro-4-[(3-methyl-4-[[1,2,4]triazolo[1,5-a]pyridin-7-yloxy]phenyl)amino]quinazolin-6-yl]-N-methylbut-2-ynamide (Compound 28) (34.7 mg, 15%) as a white solid. LCMS (ESI, m/z): [M+H]⁺=482.1. ¹H NMR (300 MHz, DMSO-d₆): δ 9.33 (s, 1H), 8.95 (d, J=7.5 Hz, 1H), 8.63 (d, J=7.5 Hz, 1H), 8.40 (s, 1H), 8.02-7.86 (m, 1H), 7.77-7.68 (m, 3H), 7.25-7.21 (m, 1H), 7.06-7.03 (m, 1H), 6.81 (d, J=2.7 Hz, 1H), 3.56 (s, 1H), 3.26 (s, 2H), 2.20-1.15 (m, 4H), 1.75 (s, 2H).

### Example S29. Synthesis of N-[4-([4-[(5-methylpyrazin-2-yl)methyl]phenyl]amino)quinazolin-6-yl]but-2-ynamide (Compound 29)

### Step 1: Synthesis of tert-butyl N-[4-[hydroxy(5-methylpyrazin-2-yl)methyl]phenyl]carbamate

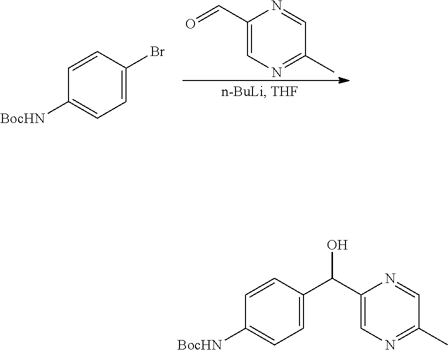

To a solution of tert-butyl N-(4-bromophenyl)carbamate (5.1 g, 18.74 mmol) in THF (60.0 mL) was added n-BuLi (15.0 mL, 2.5 mol/L) at −70° C. under N₂. The resulting mixture was stirred at −70° C. for 1 h. Then a solution of 5-methylpyrazine-2-carbaldehyde (2.5 g, 20.47 mmol) in THF (20.0 mL) was added dropwise to the mixture at −70° C. The mixture was stirred at −70° C. for another 1 h. After the reaction was completed, the mixture was quenched with NH₄Cl (aq.) and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (7/3, v/v) to afford tert-butyl N-[4-[hydroxy(5-methylpyrazin-2-yl)methyl]phenyl]carbamate (4.0 g, 67%) as a light yellow solid. LCMS (ESI, m/z): [M+H]⁺=316.1.

### Step 2: Synthesis of 4-[(5-methylpyrazin-2-yl)methyl]aniline

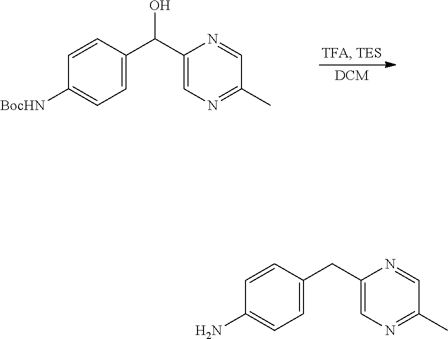 [SMILES: Cc1cnc(C(O)c2ccc(NC(=O)OC(C)(C)C)cc2)cn1 ; Cc1cnc(Cc2ccc(N)cc2)cn1]

To a solution of tert-butyl N-[4-[hydroxy(5-methylpyrazin-2-yl)methyl]phenyl]carbamate (1.0 g, 3.17 mmol) in CH₂Cl₂ (10.0 mL) was added TFA (5.0 mL) and TES (5.0 mL) at room temperature. The resulting mixture was stirred at room temperature for 6 h. After the reaction was completed, the resulting mixture was concentrated under reduced pressure. The residue was purified by flash column chromatography with CH₂Cl₂/CH₃OH (9/1, v/v) to afford 4-[(5-methylpyrazin-2-yl)methyl]aniline (200.0 mg, 31%) as a yellow solid. LCMS (ESI, m/z): [M+H]⁺=200.1.

### Step 3: Synthesis of N-[4-[(5-methylpyrazin-2-yl)methyl]phenyl]-6-nitroquinazolin-4-amine

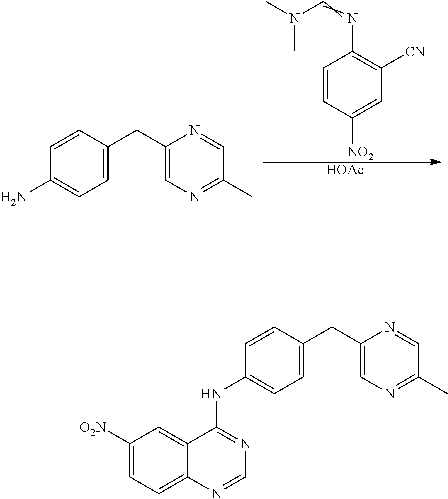 [SMILES: Cc1cnc(Cc2ccc(N)cc2)cn1 ; CN(C)C=Nc1ccc([N+](=O)[O-])cc1C#N ; Cc1cnc(Cc2ccc(Nc3ncnc4ccc([N+](=O)[O-])cc34)cc2)cn1 ; "HOAc"]

To a solution of 4-[(5-methylpyrazin-2-yl)methyl]aniline (200.0 mg, 1.0 mmol) in HOAc (5.0 mL) was added N′-(2-cyano-4-nitrophenyl)-N,N-dimethylformimidamide (219.0 mg, 1.0 mmol) at room temperature. The resulting mixture was stirred at 85° C. for 2 h. After the reaction was completed, the resulting mixture was concentrated under reduced pressure. The residue was purified by flash column chromatography with CH₂Cl₂/CH₃OH (84/16, v/v) to afford N-[4-[(5-methylpyrazin-2-yl)methyl]phenyl]-6-nitroquinazolin-4-amine (300.0 mg, 80%) as a yellow solid. LCMS (ESI, m/z): [M+H]⁺=373.1.

### Step 4: Synthesis of N4-[4-[(5-methylpyrazin-2-yl)methyl]phenyl]quinazoline-4,6-diamine

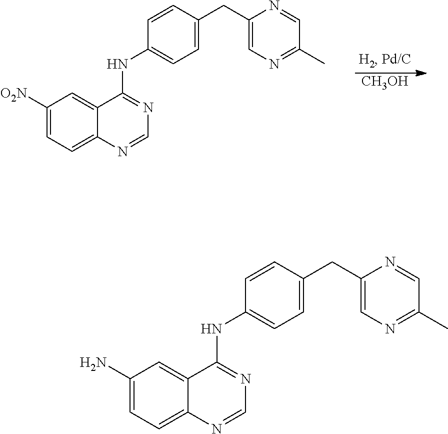

To a solution of N-[4-[(5-methylpyrazin-2-yl)methyl]phenyl]-6-nitroquinazolin-4-amine (300.0 mg, 0.81 mmol) in CH₃OH (10.0 mL) was added Pd/C (85.7 mg, dry) at room temperature. The resulting mixture was stirred at room temperature for 2 h under H₂. After the reaction was completed, the resulting mixture was filtered. The filtrate was concentrated under reduced pressure. The residue was purified by flash column chromatography with CH₂Cl₂/CH₃OH (9/1, v/v) to afford N4-[4-[(5-methylpyrazin-2-yl)methyl]phenyl]quinazoline-4,6-diamine (250.0 mg, 90%) as a yellow solid. LCMS (ESI, m/z): [M+H]⁺=343.1.

### Step 5: Synthesis of N-[4-([4-[(5-methylpyrazin-2-yl)methyl]phenyl]amino)quinazolin-6-yl]but-2-ynamide (Compound 29)

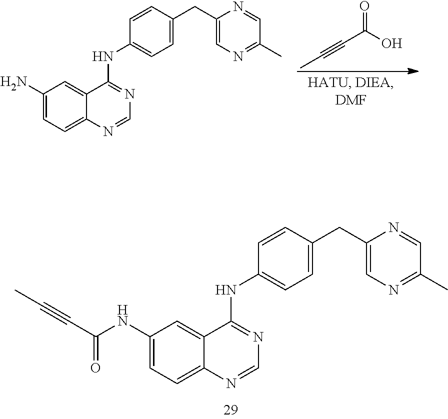

To a solution of N4-[4-[(5-methylpyrazin-2-yl)methyl]phenyl]quinazoline-4,6-diamine (200.0 mg, 0.58 mmol) and 2-butynoic acid (63.8 mg, 0.76 mmol) in DMF (5.0 mL) was added DIEA (754.9 mg, 5.84 mmol) and HATU (355.3 mg, 0.93 mmol) at room temperature. The resulting mixture was stirred at room temperature for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with acetonitrile/water (74/26, v/v) and then purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 31% B to 36 B % in 8 min; 254 nm) to afford N-[4-([4-[(5-methylpyrazin-2-yl)methyl]phenyl]amino)quinazolin-6-yl]but-2-ynamide (Compound 29) (5.8 mg, 2%) as a yellow solid. LCMS (ESI, m/z): [M+H]⁺=409.1. ¹H NMR (300 MHz, DMSO-d₆): δ 10.91 (s, 1H), 9.78 (s, 1H), 8.71 (s, 1H), 8.51-8.46 (m, 3H), 7.81-7.67 (m, 4H), 7.28 (d, J=8.4 Hz, 2H), 4.09 (s, 2H), 2.46 (s, 3H), 2.09 (s, 3H).

### Example S30. Synthesis of N-[4-[(3-fluoro-4-[[1,2,4]triazolo[1,5-a]pyridin-7-yloxy]phenyl)amino]quinazolin-6-yl]-5-hydroxypent-2-ynamide (Compound 30)

### Step 1: Synthesis of 7-(2-fluoro-4-nitrophenoxy)-[1,2,4]triazolo[1,5-a]pyridine

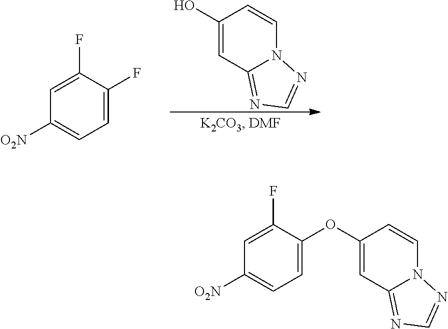 [SMILES: O=[N+]([O-])c1ccc(F)c(F)c1 ; Oc1ccn2ncnc2c1 ; O=[N+]([O-])c1ccc(Oc2ccn3ncnc3c2)c(F)c1]

To a mixture of 1,2-difluoro-4-nitrobenzene (2.0 g, 12.57 mmol) and [1,2,4]triazolo[1,5-a]pyridin-7-ol (1.7 g, 12.57 mmol) in DMF (30.0 mL) was added K₂CO₃ (3.5 g, 25.14 mmol) at room temperature. The resulting mixture was stirred at 70° C. for 2.5 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (3/7, v/v) to afford 7-(2-fluoro-4-nitrophenoxy)-[1,2,4]triazolo[1,5-a]pyridine (2.5 g, 72%) as a white solid. LCMS (ESI, m/z): [M+H]⁺=275.0.

### Step 2: Synthesis of 3-fluoro-4-[[1,2,4]triazolo[1,5-a]pyridin-7-yloxy]aniline

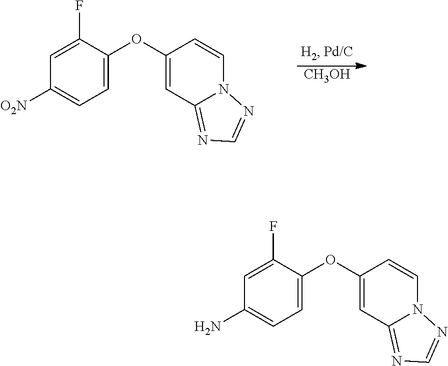 [SMILES: O=[N+]([O-])c1ccc(Oc2ccn3ncnc3c2)c(F)c1 ; "CH3OH" ; Nc1ccc(Oc2ccn3ncnc3c2)c(F)c1]

To a solution of 7-(2-fluoro-4-nitrophenoxy)-[1,2,4]triazolo[1,5-a]pyridine (2.6 g, 9.77 mmol) in MeOH (25.0 mL) was added Pd/C (800.0 mg, dry). The resulting mixture was stirred at room temperature for 3 h under H₂. After the reaction was completed, the resulting mixture was filtered. The filtrate was concentrated under vacuum to afford 3-fluoro-4-[[1,2,4]triazolo[1,5-a]pyridin-7-yloxy]aniline (1.3 g, crude) as a yellow solid. LCMS (ESI, m/z): [M+H]⁺=245.0.

### Step 3: Synthesis of N-(3-fluoro-4-[[1,2,4]triazolo[1,5-a]pyridin-7-yloxy]phenyl)-6-nitroquinazolin-4-amine

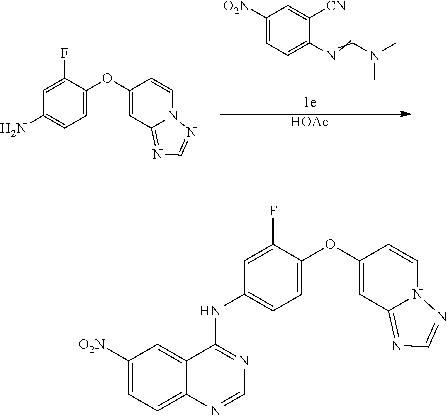 [SMILES: Nc1ccc(Oc2ccn3ncnc3c2)c(F)c1 ; CN(C)C=Nc1ccc([N+](=O)[O-])cc1C#N ; O=[N+]([O-])c1ccc2ncnc(Nc3ccc(Oc4ccn5ncnc5c4)c(F)c3)c2c1 ; "HOAc"]

A mixture of 3-fluoro-4-[[1,2,4]triazolo[1,5-a]pyridin-7-yloxy]aniline (850.0 mg, 3.48 mmol) and N′-(2-cyano-4-nitrophenyl)-N,N-dimethylformimidamide (759.4 mg, 3.48 mmol) in HOAc (15.0 mL) was stirred at 85° C. for 2 h. After the reaction was completed, the resulting mixture was concentrated under reduced pressure. The residue was purified by flash column chromatography with CH₂Cl₂/MeOH (10/1, v/v) to afford N-(3-fluoro-4-[[1,2,4]triazolo[1,5-a]pyridin-7-yloxy]phenyl)-6-nitroquinazolin-4-amine (680.0 mg, 46%) as a yellow solid. LCMS (ESI, m/z): [M+H]⁺=418.0.

### Step 4: Synthesis of N4-(3-fluoro-4-[[1,2,4]triazolo[1,5-a]pyridin-7-yloxy]phenyl)quinazoline-4,6-diamine

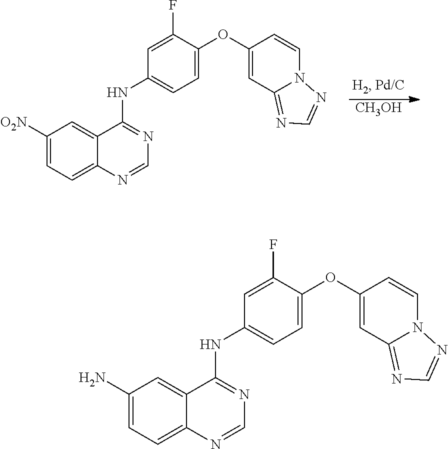

To a solution of N-(3-fluoro-4-[[1,2,4]triazolo[1,5-a]pyridin-7-yloxy]phenyl)-6-nitroquinazolin-4-amine (680.0 mg, 1.62 mmol) in MeOH (15.0 mL) was added Pd/C (247.9 mg, dry). The resulting mixture was stirred at room temperature for 3 h under H₂. After the reaction was completed, the resulting mixture was filtered. The filtrate was concentrated under vacuum to afford N4-(3-fluoro-4-[[1,2,4]triazolo[1,5-a]pyridin-7-yloxy]phenyl)quinazoline-4,6-diamine (340.0 mg, crude) as a light yellow solid. LCMS (ESI, m/z): [M+H]⁺=3 88.0.

### Step 5: Synthesis of propyl 5-(oxan-2-yloxy)pent-2-ynoate

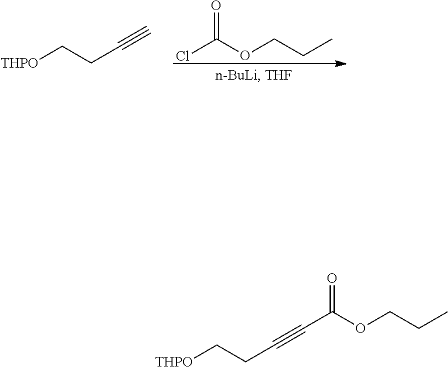

To a solution of 2-(but-3-yn-1-yloxy)oxane (12.0 g, 77.81 mmol) in THF (150.0 mL) was added n-BuLi (35.0 mL, 2.5 mol/L) at −70° C. under N₂. The mixture was stirred at −70° C. for 1 h. Then propyl chloroformate (12.0 g, 97.91 mmol) was added dropwise to the mixture at −70° C. under N₂. The mixture was stirred at −70° C. for another 1 h. After the reaction was completed, the reaction mixture was quenched with saturated NH₄Cl solution and then extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (10/1, v/v) to afford to afford propyl 5-(oxan-2-yloxy)pent-2-ynoate (14.0 g, 74%) as a colorless oil.

### Step 6: Synthesis of N-[4-[(3-fluoro-4-[[1,2,4]triazolo[1,5-a]pyridin-7-yloxy]phenyl)amino]quinazolin-6-yl]-5-(oxan-2-yloxy)pent-2-ynamide

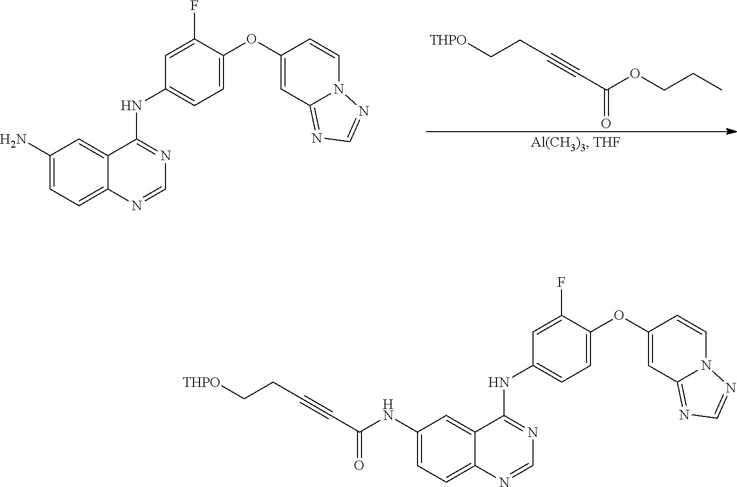

To a mixture of N4-(3-fluoro-4-[[1,2,4]triazolo[1,5-a]pyridin-7-yloxy]phenyl)quinazoline-4,6-diamine (260.0 mg, 0.67 mmol) and propyl 5-(oxan-2-yloxy)pent-2-ynoate (483.8 mg, 2.01 mmol) in THF (8.0 mL) was added trimethylaluminium (1.7 mL, 2 mol/L) at 0° C. The resulting mixture was stirred at 80° C. for 3 h. After the reaction was completed, the reaction mixture was quenched with sat. NH₄Cl (aq.) at room temperature. The mixture was extracted with CH₂Cl₂. The combined organic layer was washed with brine, dried over anhydrous Na₂SO₄ and filtered. The filtrate was concentrated under vacuum. The residue was purified by flash column chromatography with CH₂Cl₂/MeOH (9/1, v/v) to afford N-[4-[(3-fluoro-4-[[1,2,4]triazolo[1,5-a]pyridin-7-yloxy]phenyl)amino]quinazolin-6-yl]-5-(oxan-2-yloxy)pent-2-ynamide (180.0 mg, 47%) as a light yellow solid. LCMS (ESI, m/z): [M+H]⁺=568.2.

### Step 7: Synthesis of N-[4-[(3-fluoro-4-[[1,2,4]triazolo[1,5-a]pyridin-7-yloxy]phenyl)amino]quinazolin-6-yl]-5-hydroxypent-2-ynamide (Compound 30)

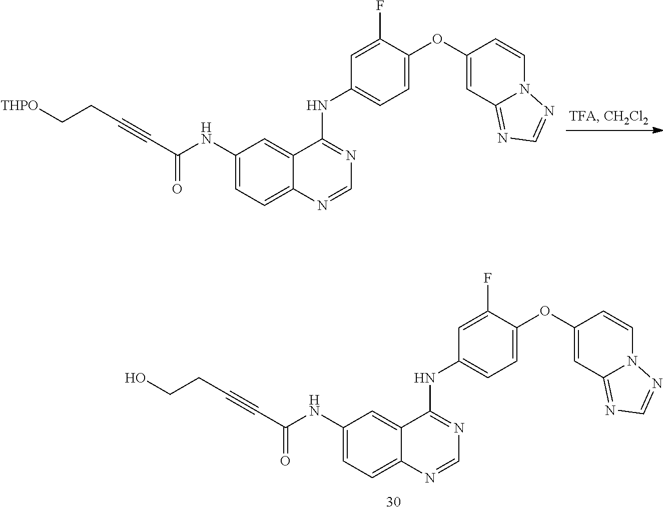

A mixture of N-[4-[(3-fluoro-4-[[1,2,4]triazolo[1,5-a]pyridin-7-yloxy]phenyl)amino]quinazolin-6-yl]-5-(oxan-2-yloxy)pent-2-ynamide (180.0 mg, 0.31 mmol) and TFA (2.0 mL) in CH₂Cl₂ (2.0 mL) was stirred at room temperature for 2 h. After the reaction was completed, the pH value of the mixture was adjusted to pH 7 with saturated NaHCO₃(aq.). The resulting mixture was extracted with ethyl acetate. The combined organic layer was washed with brine, dried over anhydrous sodium sulfate and filtered. The filtrate was concentrated under vacuum. The residue was purified by reverse phase flash column chromatography with ACN in water (5% to 100% gradient in 40 min) to afford N-[4-[(3-fluoro-4-[[1,2,4]triazolo[1,5-a]pyridin-7-yloxy]phenyl)amino]quinazolin-6-yl]-5-hydroxypent-2-ynamide (Compound 30) (41.4 mg, 27%) as a white solid. LCMS (ESI, m/z): [M+H]⁺=484.1. 1H NMR (300 MHz, DMSO-d₆): δ 10.95 (s, 1H), 10.05 (s, 1H), 8.95 (d, J=7.5 Hz, 1H), 8.75 (s, 1H), 8.61 (s, 1H), 8.40 (s, 1H), 8.15-8.10 (m, 1H), 7.85-7.73 (m, 3H), 7.47-7.41 (m, 1H), 7.10-7.07 (m, 1H), 7.02 (d, J=2.4 Hz, 1H), 5.03-4.99 (m, 1H), 3.64-3.58 (m, 2H), 2.60-2.56 (m, 2H).

### Example S31. Synthesis of N-[4-([4-[(6-ethylpyridin-3-yl)oxy]-3-methylphenyl]amino)quinazolin-6-yl]but-2-ynamide (Compound 31)

### Step 1: Synthesis of 5-(benzyloxy)-2-bromopyridine

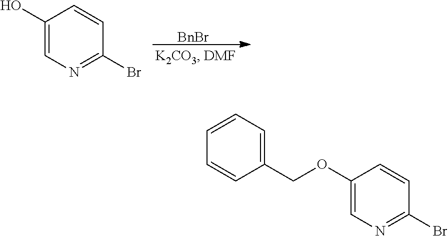 [SMILES: Oc1ccc(Br)nc1 ; "BnBr" ; Brc1ccc(OCc2ccccc2)cn1]

To a solution of 6-bromopyridin-3-ol (2.0 g, 11.49 mmol) in DMF (15.0 mL) was added K₂CO₃ (3.2 g, 22.98 mmol) and benzyl bromide (2.4 g, 13.79 mmol) at room temperature. The resulting mixture was stirred at 50° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with acetonitrile/water (64/36, v/v) to afford 5-(benzyloxy)-2-bromopyridine (3.0 g, 98%) as a grey solid. LCMS (ESI, m/z): [M+H]⁺=264.0.

### Step 2: Synthesis of 5-(benzyloxy)-2-vinylpyridine

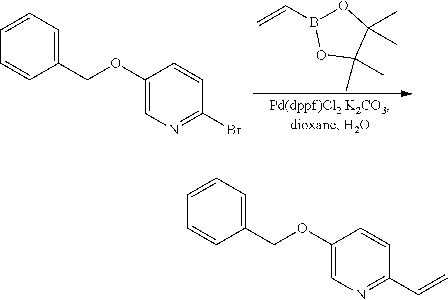 [SMILES: Brc1ccc(OCc2ccccc2)cn1 ; C=CB1OC(C)(C)C(C)(C)O1 ; C=Cc1ccc(OCc2ccccc2)cn1]

To a solution of 5-(benzyloxy)-2-bromopyridine (3.0 g, 11.36 mmol) in 1,4-dioxane/H₂O (30.0/6.0 mL) was added 2-ethenyl-4,4,5,5-tetramethyl-1,3,2-dioxaborolane (5.3 g, 34.07 mmol), K₂CO₃ (4.7 g, 34.07 mmol) and Pd(dppf)Cl₂ (831.1 mg, 1.14 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (7/3, v/v) to afford 5-(benzyloxy)-2-vinylpyridine (2.0 g, 83%) as a yellow oil. LCMS (ESI, m/z): [M+H]⁺=212.1.

### Step 3: Synthesis of 6-ethylpyridin-3-ol

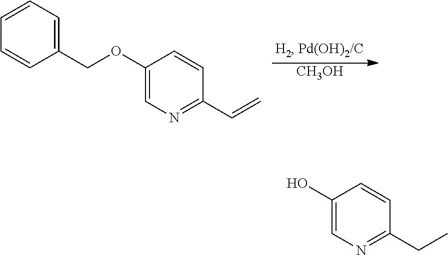

To a solution of 5-(benzyloxy)-2-vinylpyridine (1.5 g, 7.10 mmol) in CH₃OH (20.0 mL) was added Pd(OH)₂/C (997.1 mg, dry) at room temperature. The resulting mixture was stirred at room temperature for 1 h under H₂. After the reaction was completed, the resulting mixture was filtered. The filtrate was concentrated under reduced pressure. The residue was purified by reverse phase flash column chromatography with acetonitrile/water (1/9, v/v) to afford 6-ethylpyridin-3-ol (750.0 mg, 85%) as a white solid. LCMS (ESI, m/z): [M+H]⁺=124.0.

### Step 4: Synthesis of 2-ethyl-5-(2-methyl-4-nitrophenoxy)pyridine

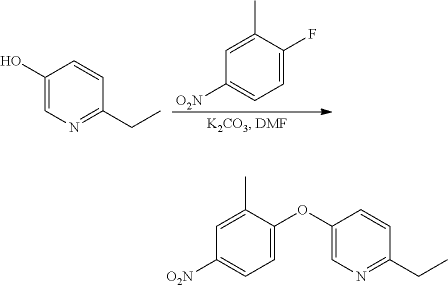

To a solution of 6-ethylpyridin-3-ol (1.0 g, 8.12 mmol) and 1-fluoro-2-methyl-4-nitrobenzene (1.5 g, 9.74 mmol) in DMF (20.0 mL) was added K₂CO₃ (2.2 g, 16.24 mmol) at room temperature. The resulting mixture was stirred at 50° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (88/12, v/v) to afford 2-ethyl-5-(2-methyl-4-nitrophenoxy)pyridine (1.3 g, 62%) as a white solid. LCMS (ESI, m/z): [M+H]⁺=259.1.

### Step 5: Synthesis of 4-[(6-ethylpyridin-3-yl)oxy]-3-methylaniline

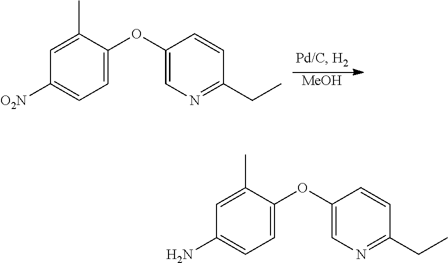 [SMILES: "MeOH" ; CCc1ccc(Oc2ccc([N+](=O)[O-])cc2C)cn1 ; CCc1ccc(Oc2ccc(N)cc2C)cn1]

To a solution of 2-ethyl-5-(2-methyl-4-nitrophenoxy)pyridine (1.3 g, 5.03 mmol) in CH₃OH (20.0 mL) was added Pd/C (535.6 mg, dry) at room temperature. The resulting mixture was stirred at room temperature for 2 h under H₂. After the reaction was completed, the resulting mixture was filtered. The filtrate was concentrated under reduced pressure. The residue was purified by flash column chromatography with CH₂Cl₂/CH₃OH (9/1, v/v) to afford 4-[(6-ethylpyridin-3-yl)oxy]-3-methylaniline (1.0 g, 87%) as a yellow oil. LCMS (ESI, m/z): [M+H]⁺=229.1.

### Step 6: Synthesis of N-[4-[(6-ethylpyridin-3-yl)oxy]-3-methylphenyl]-6-nitroquinazolin-4-amine

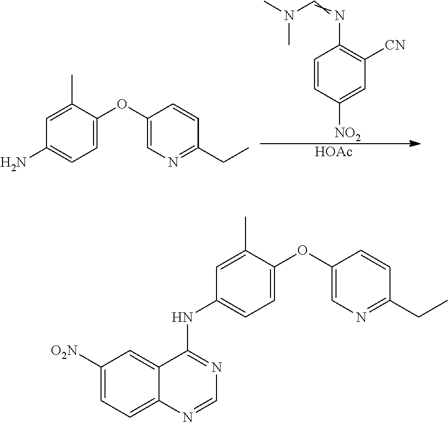 [SMILES: CCc1ccc(Oc2ccc(N)cc2C)cn1 ; CN(C)C=Nc1ccc([N+](=O)[O-])cc1C#N ; CCc1ccc(Oc2ccc(Nc3ncnc4ccc([N+](=O)[O-])cc34)cc2C)cn1 ; "HOAc"]

To a solution of 4-[(6-ethylpyridin-3-yl)oxy]-3-methylaniline (1.0 g, 4.38 mmol) in AcOH (10.0 mL) was added N′-(2-cyano-4-nitrophenyl)-N,N-dimethylformimidamide (955.8 mg, 4.38 mmol) at room temperature. The resulting mixture was stirred at 85° C. for 2 h. After the reaction was completed, the resulting mixture was concentrated under reduced pressure. The residue was purified by flash column chromatography with CH₂Cl₂/CH₃OH (9/1, v/v) to afford N-[4-[(6-ethylpyridin-3-yl)oxy]-3-methylphenyl]-6-nitroquinazolin-4-amine (1.5 g, 85%) as a yellow solid. LCMS (ESI, m/z): [M+H]⁺=402.1.

### Step 7: Synthesis of N4-[4-[(6-ethylpyridin-3-yl)oxy]-3-methylphenyl]quinazoline-4,6-diamine

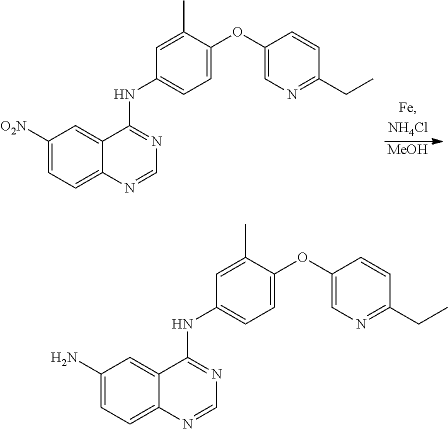 [SMILES: "MeOH" ; CCc1ccc(Oc2ccc(Nc3ncnc4ccc([N+](=O)[O-])cc34)cc2C)cn1 ; CCc1ccc(Oc2ccc(Nc3ncnc4ccc(N)cc34)cc2C)cn1]

To a solution of N-[4-[(6-ethylpyridin-3-yl)oxy]-3-methylphenyl]-6-nitroquinazolin-4-amine 1.50 g, 3.74 mmol) in CH₃OH/H₂O (10.0/2.0 mL) was added Fe (2.09 g, 37.37 mmol) and NH₄Cl (2.0 g, 37.37 mmol) at room temperature. The resulting mixture was stirred at 80° C. for 1 h. After the reaction was completed, the resulting mixture was filtered. The filtrate was concentrated under reduced pressure. The residue was purified by flash column chromatography with CH₂Cl₂/CH₃OH (82/18, v/v) to afford N4-[4-[(6-ethylpyridin-3-yl)oxy]-3-methylphenyl]quinazoline-4,6-diamine (1.0 g, 72%) as a yellow solid. LCMS (ESI, m/z): [M+H]⁺=372.1.

### Step 8: Synthesis of N-[4-([4-[(6-ethylpyridin-3-yl)oxy]-3-methylphenyl]amino)quinazolin-6-yl]but-2-ynamide (Compound 31)

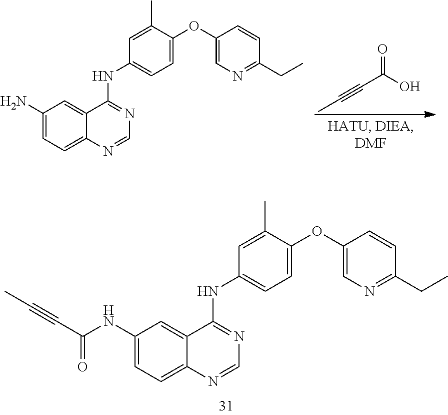

To a solution of N4-[4-[(6-ethylpyridin-3-yl)oxy]-3-methylphenyl]quinazoline-4,6-diamine (500.0 mg, 1.35 mmol) in DMF (5.0 mL) was added 2-butynoic acid (147.1 mg, 1.75 mmol), DIEA (1.74 g, 13.46 mmol) and HATU (818.9 mg, 2.15 mmol) at room temperature. The resulting mixture was stirred at room temperature for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH. (86/14, v/v) and then purified by Prep-HPLC Column: Xselect CSH OBD Column 30×150 mm, 5 um; Mobile Phase A: Water (0.1% FA), Mobile Phase B: ACN; Flow rate: 60 mL/min; Gradient: 18% B to 28% B in 8 min; 254 nm) to afford N-[4-([4-[(6-ethylpyridin-3-yl)oxy]-3-methylphenyl]amino)quinazolin-6-yl]but-2-ynamide (Compound 31) (46.0 mg, 8%) as a yellow solid. LCMS (ESI, m/z): [M+H]⁺=438.1. ¹H NMR (300 MHz, DMSO-d₆): δ 11.03 (s, 1H), 10.51 (s, 1H), 8.86 (s, 1H), 8.64 (s, 1H), 8.23 (s, 1H), 7.86-7.76 (m, 2H), 7.66 (s, 1H), 7.58-7.54 (m, 1H), 7.29-7.23 (m, 2H), 6.97 (d, J=8.7 Hz, 1H), 2.76-2.69 (m, 2H), 2.22 (s, 3H), 2.08 (s, 3H), 1.23-1.11 (m, 3H).

### Example S32. Synthesis of 5-hydroxy-N-[4-[(4-[imidazo[1,2-a]pyridin-7-yloxy]-3-methylphenyl)amino]quinazolin-6-yl]pent-2-ynamide (Compound 32)

### Step 1: Synthesis of N-[4-[(4-[imidazo[1,2-a]pyridin-7-yloxy]-3-methylphenyl)amino]quinazolin-6-yl]-5-(oxan-2-yloxy)pent-2-ynamide

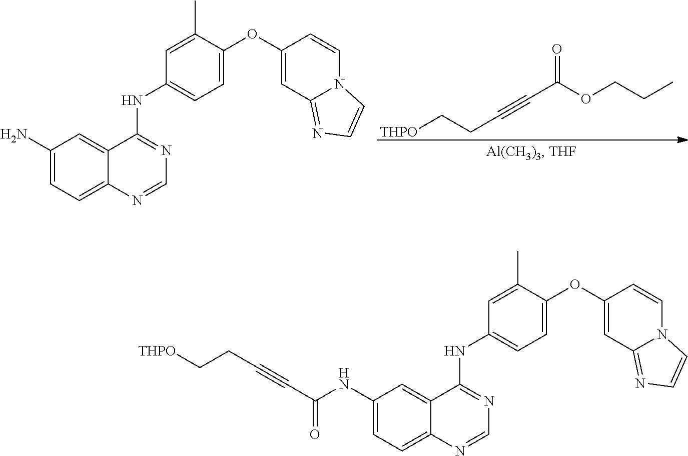

To a solution of N4-(4-[imidazo[1,2-a]pyridin-7-yloxy]-3-methylphenyl)quinazoline-4,6-diamine (450.0 mg, 1.18 mmol) in THF (5.0 mL) was added propyl 5-(oxan-2-yloxy)pent-2-ynoate (848.3 mg, 3.53 mmol) and Al(CH₃)₃ (3.0 mL, 2 mol/L in toluene) at 0° C. The resulting mixture was stirred at 80° C. for 4 h. After the reaction was completed, the reaction mixture was quenched with NH₄Cl (aq.) at room temperature and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH. (9/1, v/v) to afford N-[4-[(4-[imidazo[1,2-a]pyridin-7-yloxy]-3-methylphenyl)amino]quinazolin-6-yl]-5-(oxan-2-yloxy)pent-2-ynamide (180.0 mg, 27%) as a yellow solid. LCMS (ESI, m/z): [M+H]⁺=563.2.

### Step 2: Synthesis of 5-hydroxy-N-[4-[(4-[imidazo[1,2-a]pyridin-7-yloxy]-3-methylphenyl)amino]quinazolin-6-yl]pent-2-ynamide (Compound 32)

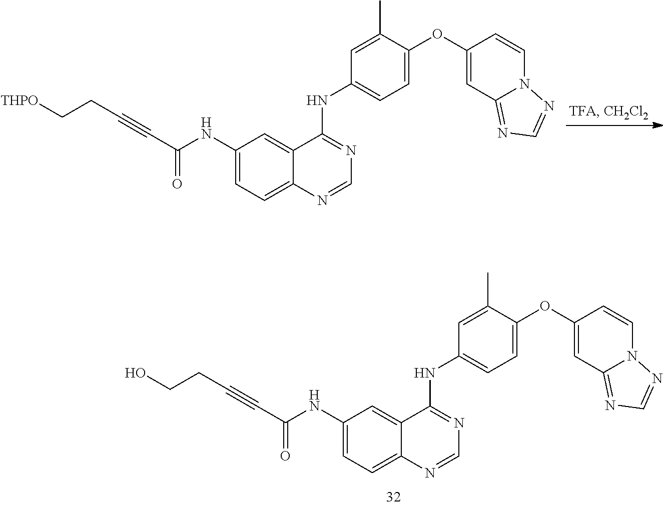

To a solution of N-[4-[(4-[imidazo[1,2-a]pyridin-7-yloxy]-3-methylphenyl)amino]quinazolin-6-yl]-5-(oxan-2-yloxy)pent-2-ynamide (150.0 mg, 0.27 mmol) in CH₂Cl₂ (2.0 mL) was added TFA (2.0 mL) at room temperature. The resulting mixture was stirred at room temperature for 2 h. After the reaction was completed, the mixture was basified to pH 8 with NaHCO₃(aq.) and then extracted with CH₂Cl₂. The combined organic layer was washed with brine, dried over anhydrous sodium sulfate and filtered. The filtrate was concentrated under reduced pressure. The residue was purified by flash column chromatography with CH₂Cl₂/CH₃OH (9/1, v/v) and then purified by Prep-HPLC with the following conditions Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 31% B to 41% B in 7 min; 254 nm) to afford 5-hydroxy-N-[4-[(4-[imidazo[1,2-a]pyridin-7-yloxy]-3-methylphenyl)amino]quinazolin-6-yl]pent-2-ynamide (Compound 32) (32.8 mg, 25%) as a white solid. LCMS (ESI, m/z): [M+H]⁺=479.1. ¹H NMR (300 MHz, DMSO-d₆): δ 10.93 (s, 1H), 9.86 (s, 1H) 8.74 (s, 1H), 8.57-8.54 (m, 2H), 7.85-7.72 (m, 5H), 7.44 (d, J=1.2 Hz, 1H), 7.14 (d, J=8.7 Hz, 1H), 6.83-6.80 (m, 1H), 6.55 (d, J=2.4 Hz, 1H), 5.07-4.99 (m, 1H), 3.67-3.61 (m, 2H), 2.62-2.58 (m, 2H), 2.20 (s, 3H).

### Example S33. Synthesis of N-[4-[(3-chloro-4-[[1,2,4]triazolo[1,5-a]pyridin-7-yloxy]phenyl)amino]quinazolin-6-yl]-5-hydroxypent-2-ynamide (Compound 33)

### Step 1: Synthesis of N-(3-chloro-4-[[1,2,4]triazolo[1,5-a]pyridin-7-yloxy]phenyl)-6-nitroquinazolin-4-amine

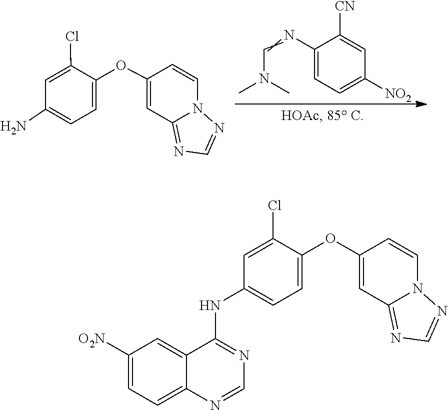

A solution of 3-chloro-4-([1,2,4]triazolo[1,5-a]pyridin-7-yloxy)aniline (500.0 mg, 1.92 mmol) and N′-(2-cyano-4-nitrophenyl)-N,N-dimethylformimidamide (502.3 mg, 2.30 mmol) in HOAc (15.0 mL) was stirred at 85° C. for 2 h. After the reaction was completed, the resulting mixture was concentrated under vacuum. The residue was purified by flash column chromatography with CH₂Cl₂/CH₃OH (96/4, v/v) to afford N-(3-chloro-4-[[1,2,4]triazolo[1,5-a]pyridin-7-yloxy]phenyl)-6-nitroquinazolin-4-amine (800.0 mg, 96%) as a yellow solid. LCMS (ESI, m/z): [M+H]⁺=434.1.

### Step 2: Synthesis of N4-(3-chloro-4-[[1,2,4]triazolo[1,5-a]pyridin-7-yloxy]phenyl)quinazoline-4,6-diamine

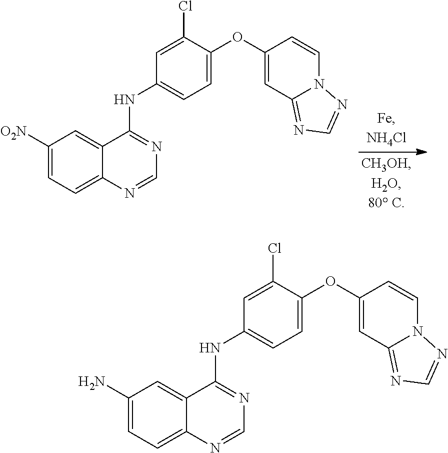 [SMILES: O=[N+]([O-])c1ccc2ncnc(Nc3ccc(Oc4ccn5ncnc5c4)c(Cl)c3)c2c1 ; Nc1ccc2ncnc(Nc3ccc(Oc4ccn5ncnc5c4)c(Cl)c3)c2c1]

A mixture of N-(3-chloro-4-[[1,2,4]triazolo[1,5-a]pyridin-7-yloxy]phenyl)-6-nitroquinazolin-4-amine (700.0 mg, 1.61 mmol), Fe (901.1 mg, 16.14 mmol) and NH₄Cl (863.1 mg, 16.14 mmol) in CH₃OH (30.0 mL) and H₂O (6.0 mL) was stirred at 80° C. for 2 h. After the reaction was completed, the resulting mixture was filtered. The filtrate was concentrated under vacuum. The residue was purified by flash column chromatography with CH₂Cl₂/CH₃OH (92/8, v/v) to afford N4-(3-chloro-4-[[1,2,4]triazolo[1,5-a]pyridin-7-yloxy]phenyl)quinazoline-4,6-diamine (400.0 mg, 61%) as a yellow oil. LCMS (ESI, m/z): [M+H]⁺=404.1.

### Step 3: Synthesis of N-[4-[(3-chloro-4-[[1,2,4]triazolo[1,5-a]pyridin-7-yloxy]phenyl)amino]quinazolin-6-yl]-5-(oxan-2-yloxy)pent-2-ynamide

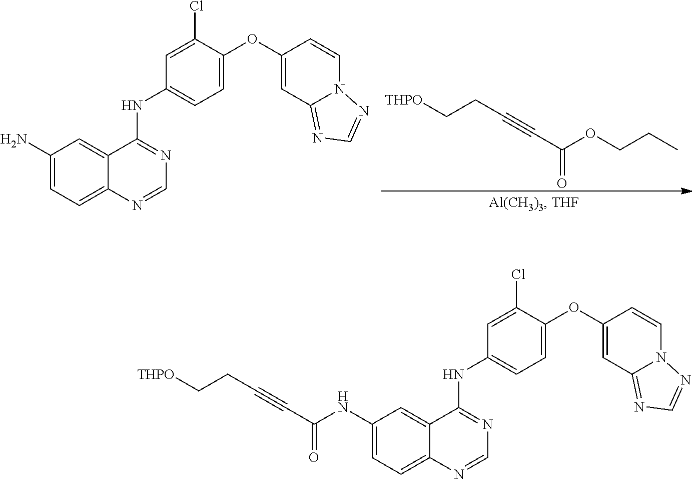

To a mixture of N4-(3-chloro-4-[[1,2,4]triazolo[1,5-a]pyridin-7-yloxy]phenyl)quinazoline-4,6-diamine (300.0 mg, 0.74 mmol) and propyl 5-(oxan-2-yloxy)pent-2-ynoate (535.5 mg, 2.23 mmol) in THF (10.0 mL) was added Al(CH₃)₃ (1.9 mL, 2 mol/L in toluene) dropwise at 0° C. The resulting mixture was stirred at 80° C. for 3 h. After the reaction was completed, the reaction mixture was quenched with saturated NH₄Cl solution at room temperature. The resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (93/7, v/v) to afford N-[4-[(3-chloro-4-[[1,2,4]triazolo[1,5-a]pyridin-7-yloxy]phenyl)amino]quinazolin-6-yl]-5-(oxan-2-yloxy)pent-2-ynamide (222.0 mg, 51%) as a yellow solid. LCMS (ESI, m/z): [M+H]⁺=584.2.

### Step 4: Synthesis of N-[4-[(3-chloro-4-[[1,2,4]triazolo[1,5-a]pyridin-7-yloxy]phenyl)amino]quinazolin-6-yl]-5-hydroxypent-2-ynamide (Compound 33)

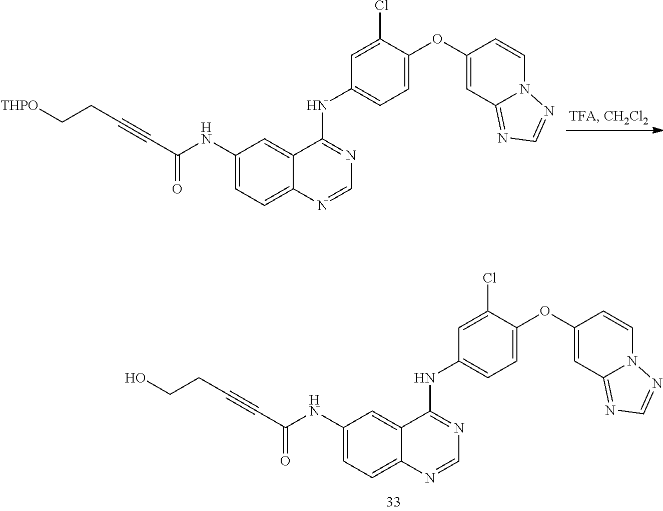

A mixture of N-[4-[(3-chloro-4-[[1,2,4]triazolo[1,5-a]pyridin-7-yloxy]phenyl)amino]quinazolin-6-yl]-5-(oxan-2-yloxy)pent-2-ynamide (220.0 mg, 0.37 mmol) and TFA (5.0 mL) in CH₂Cl₂ (5.0 mL) was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (89/11, v/v) and then purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 31% B to 41% B in 8 min; 254/220 nm) to afford N-[4-[(3-chloro-4-[[1,2,4]triazolo[1,5-a]pyridin-7-yloxy]phenyl)amino]quinazolin-6-yl]-5-hydroxypent-2-ynamide (Compound 33) (59.0 mg, 32%). LCMS (ESI, m/z): [M+H]⁺=500.2. ¹H NMR (300 MHz, DMSO-d₆): δ 10.98 (s, 1H), 10.06 (s, 1H), 8.97 (d, J=7.8 Hz, 1H), 8.78 (s, 1H), 8.63 (s, 1H), 8.42 (s, 1H), 8.28 (d, J=2.4 Hz, 1H), 7.97-7.93 (m, 1H), 7.87-7.80 (m, 2H), 7.47 (d, J=8.7 Hz, 1H), 7.10-7.06 (m, 1H), 6.95 (d, J=2.1 Hz, 1H), 5.05-5.02 (m, 1H), 3.67-3.60 (m, 2H), 2.62-2.58 (m, 2H).

### Example S34. Synthesis of 5-hydroxy-N-[4-([3-methyl-4-[(6-methylpyridin-3-yl)oxy]phenyl]amino)quinazolin-6-yl]pent-2-ynamide (Compound 34)

### Step 1: Synthesis of N-[4-([3-methyl-4-[(6-methylpyridin-3-yl)oxy]phenyl]amino)quinazolin-6-yl]-5-(oxan-2-yloxy)pent-2-ynamide

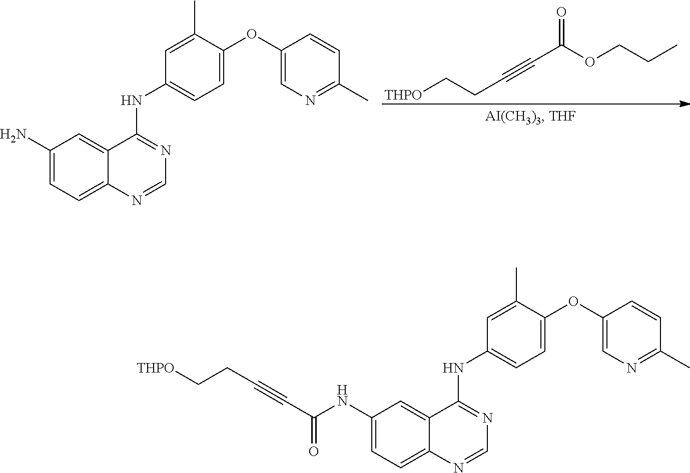

To a mixture of N4-[3-methyl-4-[(6-methylpyridin-3-yl)oxy]phenyl]quinazoline-4,6-diamine (300.0 mg, 0.84 mmol) and propyl 5-(oxan-2-yloxy)pent-2-ynoate (605.1 mg, 2.52 mmol) in THF (10.0 mL) was added dropwise trimethylaluminium (2.1 mL, 2 mol/L in toluene) at 0° C. The resulting mixture was stirred at 80° C. for 3 h. After the reaction was completed, the reaction was quenched with sat NH₄Cl at room temperature. the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford N-[4-([3-methyl-4-[(6-methylpyridin-3-yl)oxy]phenyl]amino)quinazolin-6-yl]-5-(oxan-2-yloxy)pent-2-ynamide (400.0 mg, 88%) as a yellow solid. LCMS (ESI, m/z): [M+H]⁺=538.2.

### Step 2: Synthesis of 5-hydroxy-N-[4-([3-methyl-4-[(6-methylpyridin-3-yl)oxy]phenyl]amino)quinazolin-6-yl]pent-2-ynamide (Compound 34)

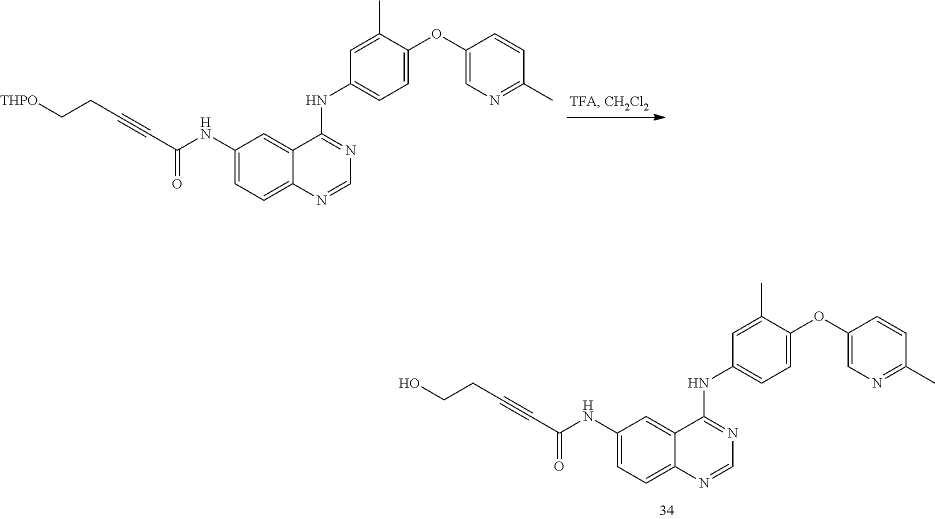

A mixture of N-[4-([3-methyl-4-[(6-methylpyridin-3-yl)oxy]phenyl]amino)quinazolin-6-yl]-5-(oxan-2-yloxy)pent-2-ynamide (300.0 mg, 0.56 mmol) and TFA (5.0 mL) in CH₂Cl₂ (5.0 mL) was stirred at room temperature for 2 h. After the reaction was completed, the resulting mixture was concentrated under vacuum. The residue was purified by flash column chromatography with CH₂Cl₂/CH₃OH (94/6, v/v) and then purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 34% B to 44% B in 8 min; 254/220 nm) to afford 5-hydroxy-N-[4-([3-methyl-4-[(6-methylpyridin-3-yl)oxy]phenyl]amino)quinazolin-6-yl]pent-2-ynamide (Compound 34) (42.6 mg, 16%) as a yellow solid. LCMS (ESI, m/z): [M+H]⁺=454.2. ¹H NMR (300 MHz, DMSO-d₆): δ 10.91 (s, 1H), 9.81 (s, 1H), 8.72 (s, 1H), 8.52 (s, 1H), 8.19 (d, J=1.8 Hz, 1H), 7.83-7.74 (m, 3H), 7.67-7.63 (m, 1H), 7.27-7.19 (m, 2H), 6.96 (d, J=8.7 Hz, 1H), 5.04-5.00 (m, 1H), 3.66-3.60 (m, 2H), 2.62-2.58 (m, 2H), 2.45 (s, 3H), 2.21 (s, 3H).

### Example S35. Synthesis of N-[4-[(4-[[1-(difluoromethyl)-2-oxopyridin-4-yl]oxy]-3-methylphenyl)amino]quinazolin-6-yl]-5-hydroxypent-2-ynamide (Compound 35)

### Step 1: Synthesis of N-[4-[(4-[[1-(difluoromethyl)-2-oxopyridin-4-yl]oxy]-3-methylphenyl)amino]quinazolin-6-yl]-5-(oxan-2-yloxy)pent-2-ynamide

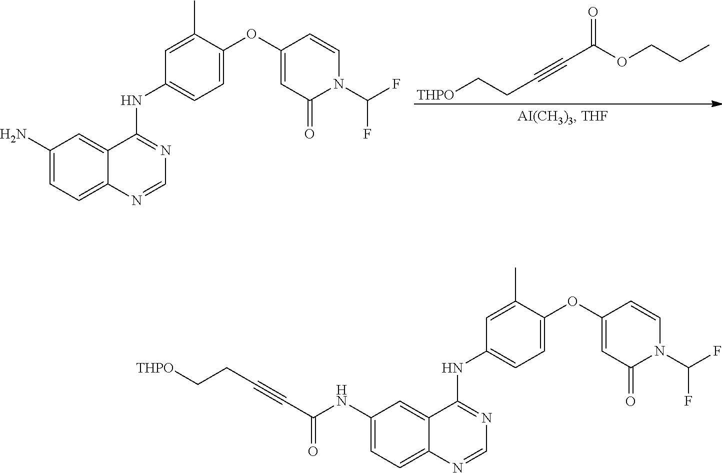

To a mixture of 4-[4-[(6-aminoquinazolin-4-yl)amino]-2-methylphenoxy]-1-(difluoromethyl)pyridin-2-one (390.0 mg, 0.95 mmol) and propyl 5-(oxan-2-yloxy)pent-2-ynoate (686.7 mg, 2.85 mmol) in THF (15.0 mL) was added trimethylaluminium (2.4 mL, 2 mol/L in toluene) dropwise at 0° C. The resulting mixture was stirred at 80° C. for 3 h. After the reaction was completed, the reaction mixture was quenched with saturated NH₄Cl (aq.) at room temperature. The resulting mixture was extracted with CH₂Cl₂. The combined organic layer was washed with water, dried over anhydrous Na₂SO₄ and filtered. The filtrate was concentrated under reduced pressure. The residue was purified by flash column chromatography with CH₂Cl₂/CH₃OH (10/1, v/v) to afford N-[4-[(4-[[1-(difluoromethyl)-2-oxopyridin-4-yl]oxy]-3-methylphenyl)amino]quinazolin-6-yl]-5-(oxan-2-yloxy)pent-2-ynamide (280.0 mg, 49%) as a light yellow solid. LCMS (ESI, m/z): [M+H^{]}+=590.2.

### Step 2: Synthesis of N-[4-[(4-[[1-(difluoromethyl)-2-oxopyridin-4-yl]oxy]-3-methylphenyl)amino]quinazolin-6-yl]-5-hydroxypent-2-ynamide (Compound 35)

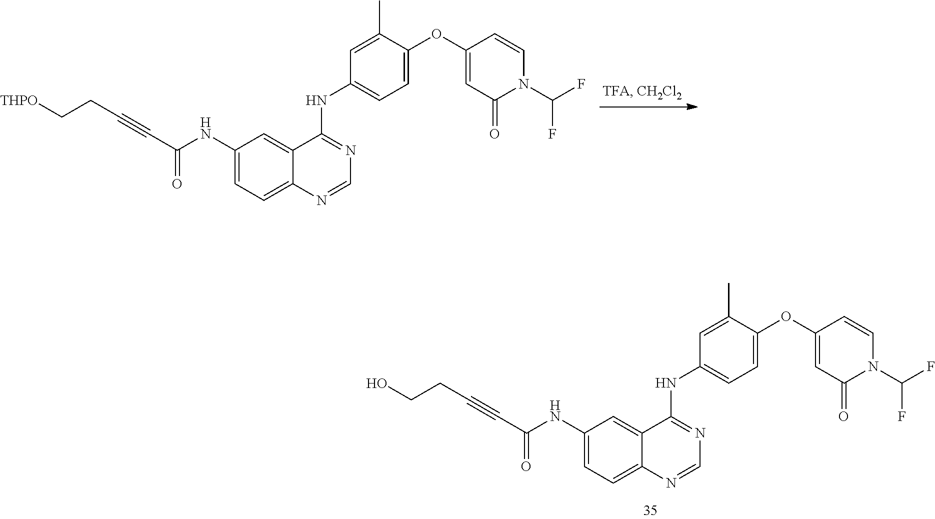

A mixture of N-[4-[(4-[[1-(difluoromethyl)-2-oxopyridin-4-yl]oxy]-3-methylphenyl)amino]quinazolin-6-yl]-5-(oxan-2-yloxy)pent-2-ynamide (280.0 mg, 0.47 mmol) and TFA (3.0 mL) in CH₂Cl₂ (3.0 mL) was stirred at room temperature for 2 h. After the reaction was completed, the pH value of the mixture was adjusted to pH 7 with saturated NaHCO₃(aq.). The resulting mixture was extracted with CH₂Cl₂. The combined organic layer was washed with brine, dried over anhydrous sodium sulfate and filtered. The filtrate was concentrated under vacuum. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 35% B to 43% B in 9 min; 254/220 nm) to afford N-[4-[(4-[[1-(difluoromethyl)-2-oxopyridin-4-yl]oxy]-3-methylphenyl)amino]quinazolin-6-yl]-5-hydroxypent-2-ynamide (Compound 35) (123.5 mg, 51%) as a white solid. LCMS (ESI, m/z): [M+H]⁺=506.2. ¹H NMR (300 MHz, DMSO-d₆): δ 10.91 (s, 1H), 9.86 (s, 1H), 8.72 (s, 1H), 8.53 (s, 1H), 7.98-7.58 (m, 6H), 7.16 (d, J=8.7 Hz, 1H), 6.37-6.33 (m, 1H), 5.34 (d, J=2.4 Hz, 1H), 5.02-4.99 (m, 1H), 3.64-3.58 (m, 2H), 2.60-2.55 (m, 2H), 2.14 (s, 3H).

### Example S36. Synthesis of 5-hydroxy-N-[4-[(3-methyl-4-[[6-(trifluoromethyl)pyridin-3-yl]oxy]phenyl)amino]quinazolin-6-yl]pent-2-ynamide (Compound 36)

### Step 1: Synthesis of N-[4-[(3-methyl-4-[[6-(trifluoromethyl)pyridin-3-yl]oxy]phenyl)amino]quinazolin-6-yl]-5-(oxan-2-yloxy) pent-2-ynamide

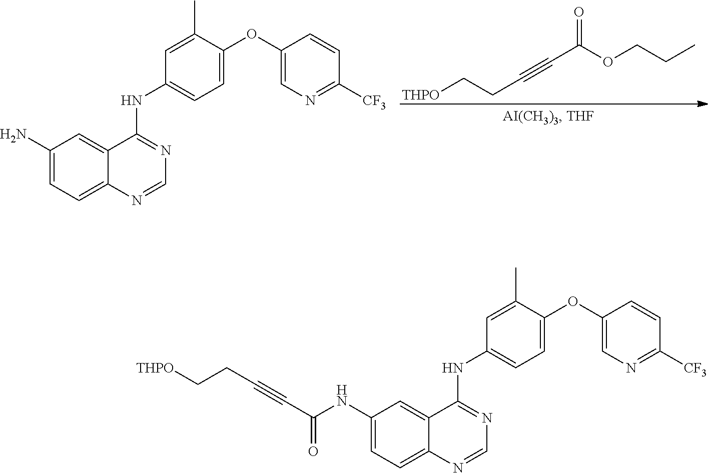

To a mixture of N4-(3-methyl-4-[[6-(trifluoromethyl)pyridin-3-yl]oxy]phenyl)quinazoline-4,6-diamine (250.0 mg, 0.48 mmol) and propyl 5-(oxan-2-yloxy)pent-2-ynoate (350.5 mg, 1.46 mmol) in THF (10.0 mL) was added dropwise Al(CH₃)₃ (1.2 mL, 2 mol/L) at 0° C. The resulting mixture was stirred at 80° C. for 3 h. After the reaction was completed, the reaction was quenched with NH₄Cl (aq) at 0° C. The resulting mixture was diluted with H₂O and extracted with DCM. The combined organic layer was washed with brine, dried over anhydrous sodium sulfate and filtered. The filtrate was concentrated under reduced pressure. The residue was purified by flash column chromatography with MeOH/CH₂Cl₂ (1/10, v/v) to afford N-[4-[(3-methyl-4-[[6-(trifluoromethyl)pyridin-3-yl]oxy]phenyl)amino]quinazolin-6-yl]-5-(oxan-2-yloxy)pent-2-ynamide (300.0 mg, 83%) as a white solid. LCMS (ESI, m/z): [M+H]⁺=592.2.

### Step 2: Synthesis of 5-hydroxy-N-[4-[(3-methyl-4-[[6-(trifluoromethyl)pyridin-3-yl]oxy]phenyl)amino]quinazolin-6-yl]pent-2-ynamide (Compound 36)

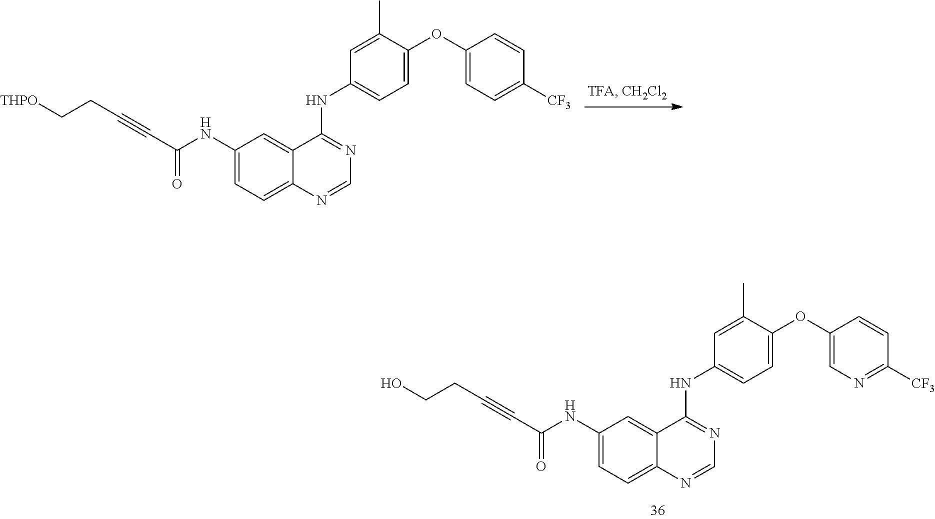

To a solution of N-[4-[(3-methyl-4-[[6-(trifluoromethyl)pyridin-3-yl]oxy]phenyl)amino]quinazolin-6-yl]-5-(oxan-2-yloxy)pent-2-ynamide (300.0 mg, 0.51 mmol) in DCM (5.0 mL) was added TFA (2.0 mL) at room temperature. The resulting mixture was stirred at room temperature for 1 h. After the reaction was completed, the mixture was concentrated under reduced pressure. The residue was purified by flash column chromatography with MeOH/DCM (1/10, v/v) and then purified by Prep-HPLC with the following conditions: Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 45% B to 55% B in 8 min; 254/220 nm; RT1:7.88 min to afford 5-hydroxy-N-[4-[(3-methyl-4-[[6-(trifluoromethyl)pyridin-3-yl]oxy]phenyl)amino]quinazolin-6-yl]pent-2-ynamide (Compound 36) (99.9 mg, 43%) as a light yellow solid. LCMS (ESI, m/z): [M+H]⁺=508.2. ¹H NMR (300 MHz, DMSO-d₆): δ 10.93 (s, 1H), 9.88 (s, 1H), 8.74 (s, 1H), 8.55 (s, 2H), 7.89 (d, J=8.7 Hz, 1H), 7.83-7.74 (m, 4H), 7.42-7.38 (m, 1H), 7.17 (d, J=8.7 Hz, 1H), 5.05-5.01 (m, 1H), 3.66-3.60 (m, 2H), 2.62-2.57 (m, 2H), 2.19 (s, 3H).

### Example S37. Synthesis of N-[4-[(3-methyl-4-[[6-(trifluoromethyl)pyridin-3-yl]oxy]phenyl)amino]quinazolin-6-yl]but-2-ynamide (Compound 37)

### Step 1: Synthesis of 5-(2-methyl-4-nitrophenoxy)-2-(trifluoromethyl)pyridine

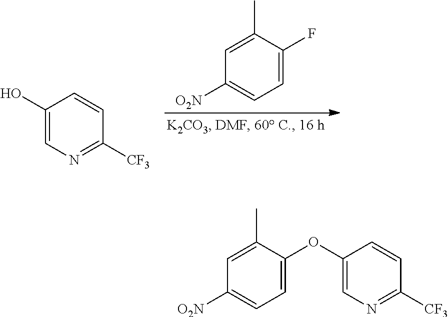 [SMILES: Oc1ccc(C(F)(F)F)nc1 ; Cc1cc([N+](=O)[O-])ccc1F ; Cc1cc([N+](=O)[O-])ccc1Oc1ccc(C(F)(F)F)nc1]

To a mixture of 6-(trifluoromethyl)pyridin-3-ol (4.5 g, 27.59 mmol) in DMF (40.0 mL) was added 1-fluoro-2-methyl-4-nitrobenzene (4.2 g, 27.59 mmol) and K₂CO₃ (11.4 g, 82.77 mmol) at room temperature. The resulting mixture was stirred at 60° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/EtOAc (2/1, v/v) to afford 5-(2-methyl-4-nitrophenoxy)-2-(trifluoromethyl)pyridine (7.0 g, 97%) as a light yellow oil. LCMS (ESI, m/z): [M+H]⁺=299.1.

### Step 2: Synthesis of 3-methyl-4-[[6-(trifluoromethyl)pyridin-3-yl]oxy]aniline

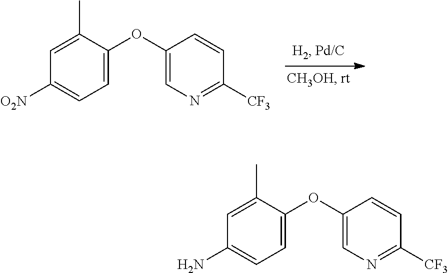 [SMILES: Cc1cc([N+](=O)[O-])ccc1Oc1ccc(C(F)(F)F)nc1 ; Cc1cc(N)ccc1Oc1ccc(C(F)(F)F)nc1]

To a solution of 5-(2-methyl-4-nitrophenoxy)-2-(trifluoromethyl)pyridine (7.0 g, 23.47 mmol) in MeOH (100.0 mL) was added Pd/C (2.1 g, dry). The mixture was stirred at room temperature for 4 h under H₂. After the reaction was completed, the reaction mixture was filtered. The filtrate was evaporated in vacuo to afford 3-methyl-4-[[6-(trifluoromethyl)pyridin-3-yl]oxy]aniline (5.0 g, crude) as a light yellow oil. LCMS (ESI, m/z): [M+H]⁺=269.1.

### Step 3: Synthesis of N-(3-methyl-4-[[6-(trifluoromethyl)pyridin-3-yl]oxy]phenyl)-6-nitroquinazolin-4-amine

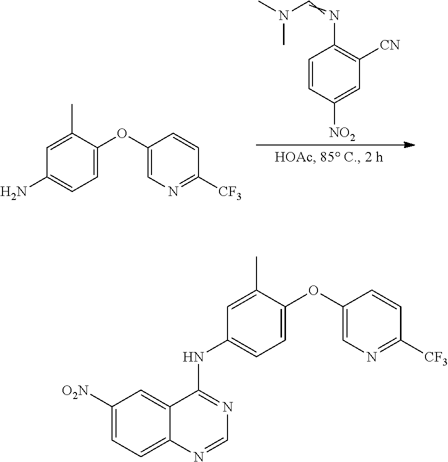 [SMILES: Cc1cc(N)ccc1Oc1ccc(C(F)(F)F)nc1 ; CN(C)C=Nc1ccc([N+](=O)[O-])cc1C#N ; Cc1cc(Nc2ncnc3ccc([N+](=O)[O-])cc23)ccc1Oc1ccc(C(F)(F)F)nc1]

To a mixture of 3-methyl-4-[[6-(trifluoromethyl)pyridin-3-yl]oxy]aniline (4.0 g, crude) in HOAc (40.0 mL) was added N′-(2-cyano-4-nitrophenyl)-N,N-dimethylformimidamide (3.2 g, 14.91 mmol) at room temperature. The resulting mixture was stirred at 85° C. for 2 h. After the reaction was completed, the mixture was purified by flash column chromatography with petroleum ether/EtOAc (1/1, v/v) to afford N-(3-methyl-4-[[6-(trifluoromethyl)pyridin-3-yl]oxy]phenyl)-6-nitroquinazolin-4-amine (4.5 g, 68%) as a yellow solid. LCMS (ESI, m/z): [M+H]⁺=442.1.

### Step 4: Synthesis of N4-(3-methyl-4-[[6-(trifluoromethyl)pyridin-3-yl]oxy]phenyl)quinazoline-4,6-diamine

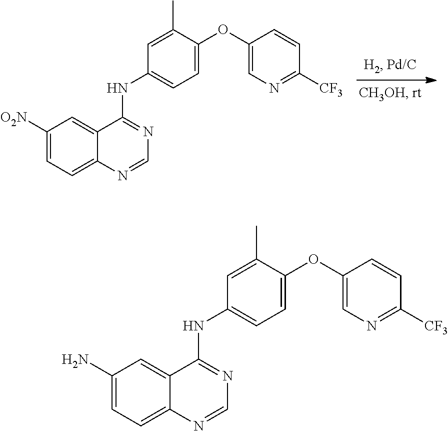 [SMILES: Cc1cc(Nc2ncnc3ccc([N+](=O)[O-])cc23)ccc1Oc1ccc(C(F)(F)F)nc1 ; Cc1cc(Nc2ncnc3ccc(N)cc23)ccc1Oc1ccc(C(F)(F)F)nc1]

To a solution of N-(3-methyl-4-[[6-(trifluoromethyl)pyridin-3-yl]oxy]phenyl)-6-nitroquinazolin-4-amine (2.0 g, 4.53 mmol) in MeOH (30.0 mL) was added Pd/C (0.6 g, dry). The mixture was stirred at room temperature for 4 h under H₂. After the reaction was completed, the reaction mixture was filtered. The filtrate was evaporated in vacuo. The residue was purified by reverse phase flash column chromatography with ACN/H₂O (60/40, v/v) to afford N4-(3-methyl-4-[[6-(trifluoromethyl)pyridin-3-yl]oxy]phenyl)quinazoline-4,6-diamine (1.4 g, 97%) as a light yellow solid. LCMS (ESI, m/z): [M+H]⁺=412.1.

### Step 5: Synthesis of N-[4-[(3-methyl-4-[[6-(trifluoromethyl)pyridin-3-yl]oxy]phenyl)amino]quinazolin-6-yl]but-2-ynamide (Compound 37)

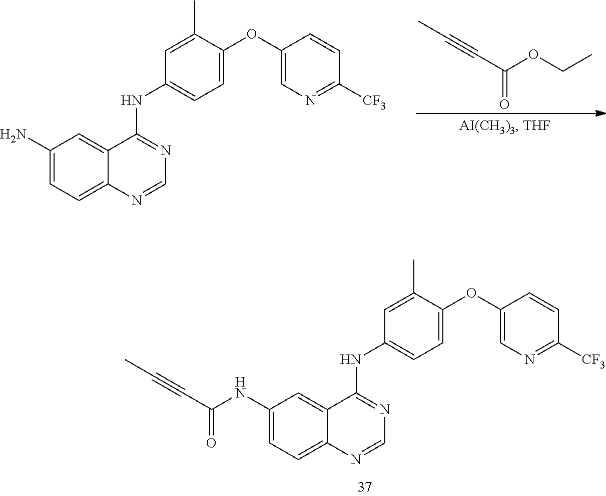

To a mixture of N4-(3-methyl-4-[[6-(trifluoromethyl)pyridin-3-yl]oxy]phenyl)quinazoline-4,6-diamine (200.0 mg, 0.48 mmol) and but-2-ynoic acid ethyl ester (163.5 mg, 1.46 mmol) in THF (10.0 mL) was added dropwise AlMe₃ (1.2 mL, 2 mol/L in toluene) at 0° C. The resulting mixture was stirred at 80° C. for 3 h. After the reaction was completed, the reaction mixture was quenched with NH₄Cl (aq) at 0° C. The resulting mixture was diluted with H₂O and extracted with DCM. The combined organic layer was washed with brine, dried over anhydrous sodium sulfate and filtered. The filtrate was concentrated under reduced pressure. The residue was purified by Prep-HPLC with the following conditions: Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 51% B to 61% B in 8 min; 254/220 nm; RTI: 7.75 min to afford N-[4-[(3-methyl-4-[[6-(trifluoromethyl)pyridin-3-yl]oxy]phenyl)amino]quinazolin-6-yl]but-2-ynamide (Compound 37) (123.0 mg, 52%) as a white solid. LCMS (ESI, m/z): [M+H]⁺=478.0. ¹H NMR (300 MHz, DMSO-d₆): δ 10.95 (s, 1H), 9.88 (s, 1H), 8.77 (s, 1H), 8.55 (s, 2H), 7.90-7.74 (m, 5H), 7.42-7.38 (m, 1H), 7.17 (d, J=8.7 Hz, 1H), 2.19 (s, 3H), 2.10 (s, 3H).

### Example S38. Synthesis of N-(4-[[4-(1-[[1,2,4]triazolo[1,5-a]pyridin-7-yl]cyclopropyl)phenyl]amino]quinazolin-6-yl)but-2-ynamide (Compound 148)

### Step 1: Synthesis of tert-butyl N-(4-acetylphenyl)carbamate

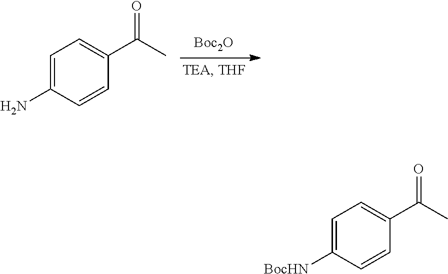

To a solution of 1-(4-aminophenyl)ethan-1-one (35.0 g, 258.94 mmol) in THF (400.0 mL) was added TEA (34.1 g, 336.62 mmol) and di-tert-butyl dicarbonate (113.0 g, 517.88 mmol) at room temperature. The resulting mixture was stirred at room temperature for 16 h. After the reaction was completed, the resulting mixture was concentrated under reduced pressure. The residue was purified by flash column chromatography with petroleum ether/ethyl acetate (1/1, v/v) to afford tert-butyl N-(4-acetylphenyl)carbamate (18.3 g, 30%) as a white solid. LCMS (ESI, m/z): [M+H]⁺=236.1.

### Step 2: Synthesis of Tert-butyl N-[4-[(1E)-1-[(4-methylbenzenesulfonamido)imino]ethyl]phenyl]carbamate

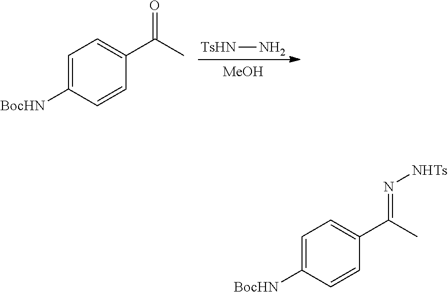

To a solution of tert-butyl N-(4-acetylphenyl)carbamate (18.3 g, 77.78 mmol) in MeOH (50.0 mL) was added 4-toluenesulfonyl hydrazide (14.5 g, 77.78 mmol) at room temperature. The resulting mixture was stirred at 60° C. for 5 h. After the reaction was completed, the resulting mixture was cooled to room temperature and filtered. The solid was collected and dried to afford tert-butyl N-[4-[(1E)-1-[(4-methylbenzenesulfonamido)imino]ethyl]phenyl]carbamate (16.9 g, crude) as a white solid. LCMS (ESI, m/z): [M+H]⁺=404.2.

### Step 3: Synthesis of Tert-butyl N-[4-(1-[[1,2,4]triazolo[1,5-a]pyridin-7-yl]ethenyl)phenyl]carbamate

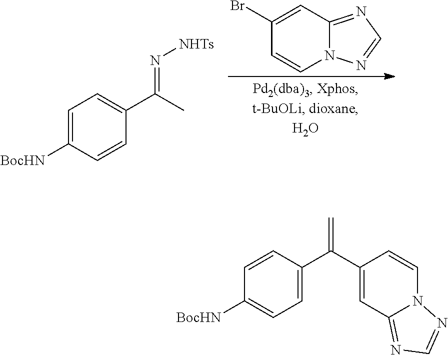

To a mixture of tert-butyl N-[4-[(1E)-1-[(4-methylbenzenesulfonamido)imino]ethyl]phenyl]carbamate (9.0 g, 22.31 mmol) in dioxane (80.0 mL)/H₂O (8.0 mL) was added 7-bromo-[1,2,4]triazolo[1,5-a]pyridine (7.5 g, 37.92 mmol), t-BuOLi (3571.2 mg, 44.61 mmol), XPhos (2126.6 mg, 4.46 mmol) and Pd₂(dba)₃ (2.0 g, 2.23 mmol) at room temperature under N₂. The resulting mixture was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford tert-butyl N-[4-(1-[[1,2,4]triazolo[1,5-a]pyridin-7-yl]ethenyl)phenyl]carbamate (5.4 g, 73%) as a light yellow solid. LCMS (ESI, m/z): [M+H]⁺=337.2.

### Step 4: Synthesis of Tert-butyl N-[4-(1-[[1,2,4]triazolo[1,5-a]pyridin-7-yl]cyclopropyl)phenyl]carbamate

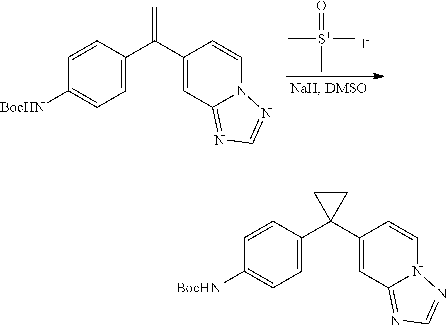

To a mixture of iodotrimethyl-lambda6-sulfanone (1.1 g, 5.05 mmol) in DMSO (20.0 mL) was added NaH (237.8 mg, 60%) at 0° C. The mixture was stirred at room temperature for 2 h. Then tert-butyl N-[4-(1-[[1,2,4]triazolo[1,5-a]pyridin-7-yl]ethenyl)phenyl]carbamate (1.0 g, 2.97 mmol) was added to the mixture. The mixture was stirred at 50° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford tert-butyl N-[4-(1-[[1,2,4]triazolo[1,5-a]pyridin-7-yl]cyclopropyl)phenyl]carbamate (550.0 mg, 52%) as a yellow solid. LCMS (ESI, m/z): [M+H]⁺=351.2.

### Step 5: Synthesis of 4-(1-[[1,2,4]triazolo[1,5-a]pyridin-7-yl]cyclopropyl)aniline

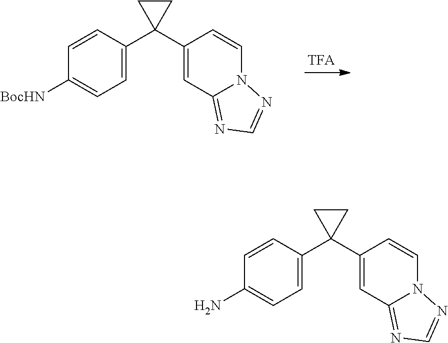 [SMILES: CC(C)(C)OC(=O)Nc1ccc(C2(c3ccn4ncnc4c3)CC2)cc1 ; Nc1ccc(C2(c3ccn4ncnc4c3)CC2)cc1]

To a solution of tert-butyl N-[4-(1-[[1,2,4]triazolo[1,5-a]pyridin-7-yl]cyclopropyl)phenyl]carbamate (550.0 mg, 1.57 mmol) and trifluoroacetaldehyde (5.0 mL) in DCM was stirred at room temperature. The mixture was stirred at room temperature for 1 h. After the reaction was completed, the reaction mixture was evaporated in vacuo. The pH value of the mixture was adjusted to 8 with saturated NaHCO₃ solution. The resulting mixture was diluted with H₂O and extracted with CH₂Cl₂. The combined organic layer was washed with brine, dried over anhydrous Na₂SO₄ and filtered. The filtrate was concentrated under reduced pressure to afford 4-(1-[[1,2,4]triazolo[1,5-a]pyridin-7-yl]cyclopropyl)aniline (450.0 mg, crude) as a light yellow solid. LCMS (ESI, m/z): [M+H]⁺=251.1.

### Step 6: Synthesis of 6-nitro-N-[4-(1-[[1,2,4]triazolo[1,5-a]pyridin-7-yl]cyclopropyl)phenyl]quinazolin-4-amine

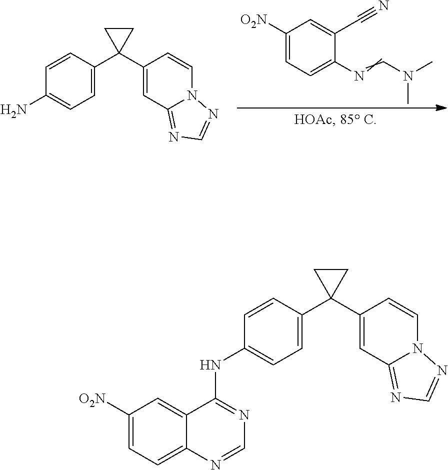 [SMILES: Nc1ccc(C2(c3ccn4ncnc4c3)CC2)cc1 ; CN(C)C=Nc1ccc([N+](=O)[O-])cc1C#N ; O=[N+]([O-])c1ccc2ncnc(Nc3ccc(C4(c5ccn6ncnc6c5)CC4)cc3)c2c1]

To a mixture of 4-(1-[[1,2,4]triazolo[1,5-a]pyridin-7-yl]cyclopropyl)aniline (410.0 mg, crude) in HOAc (15.0 mL) was added N′-(2-cyano-4-nitrophenyl)-N,N-dimethylformimidamide (357.4 mg, 1.64 mmol) at room temperature. The resulting mixture was stirred at 85° C. for 2 h. After the reaction was completed, the mixture was evaporated in vacuo. The residue was purified by flash column chromatography with petroleum ether/ethyl acetate (1/99, v/v) to afford 6-nitro-N-[4-(1-[[1,2,4]triazolo[1,5-a]pyridin-7-yl]cyclopropyl)phenyl]quinazolin-4-amine (700.0 mg, 95%) as a yellow solid. LCMS (ESI, m/z): [M+H]⁺=424.1.

### Step 7: Synthesis of N4-[4-(1-[[1,2,4]triazolo[1,5-a]pyridin-7-yl]cyclopropyl)phenyl]quinazoline-4,6-diamine

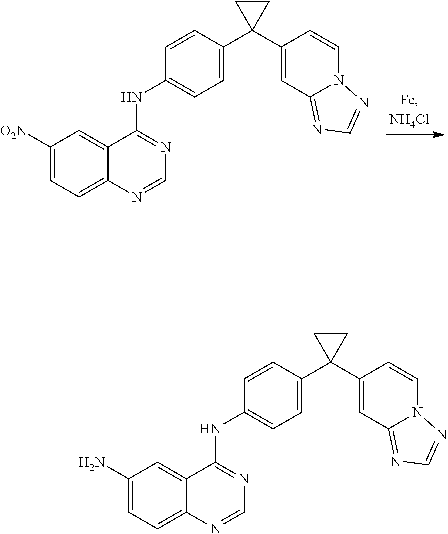 [SMILES: O=[N+]([O-])c1ccc2ncnc(Nc3ccc(C4(c5ccn6ncnc6c5)CC4)cc3)c2c1 ; Nc1ccc2ncnc(Nc3ccc(C4(c5ccn6ncnc6c5)CC4)cc3)c2c1]

To a solution of 6-nitro-N-[4-(1-[[1,2,4]triazolo[1,5-a]pyridin-7-yl]cyclopropyl)phenyl]quinazolin-4-amine (650.0 mg, 1.54 mmol) in EtOH (15.0 mL)/H₂O (3.0 mL) was added NH₄Cl (328.5 mg, 6.14 mmol). Then Fe (257.2 mg, 4.61 mmol) was added to the mixture at 80° C. The resulting mixture was stirred at 80° C. for 3 h. After the reaction was completed, the resulting mixture was cooled to room temperature and filtered. The filtrate was concentrated under reduced pressure. The residue was purified by flash column chromatography with petroleum ether/ethyl acetate (1/99, v/v) to afford N4-[4-(1-[[1,2,4]triazolo[1,5-a]pyridin-7-yl]cyclopropyl)phenyl]quinazoline-4,6-diamine (400.0 mg, 66%) as a yellow solid. LCMS (ESI, m/z): [M+H]⁺=394.2.

### Step 8: Synthesis of N-(4-[[4-(1-[[1,2,4]triazolo[1,5-a]pyridin-7-yl]cyclopropyl)phenyl]amino]quinazolin-6-yl)but-2-ynamide (Compound 148)

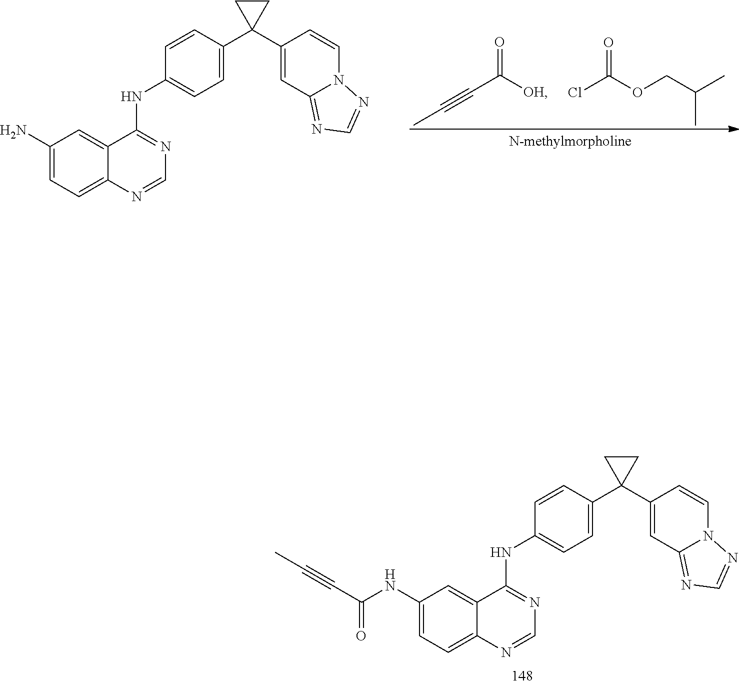

To a solution of 2-butynoic acid (188.0 mg, 2.24 mmol) in THF (10.0 mL) was added 2-methylpropyl carbonochloridate (408.2 mg, 2.99 mmol) at 0 C under N₂, the resulting mixture was stirred at 0 C for 5 min. Then N-methylmorpholine (411.3 mg, 4.07 mmol) was added dropwise to the mixture at 0 C. The mixture was stirred at 0 C for 10 min. Then a solution of N4-[4-(1-[[1,2,4]triazolo[1,5-a]pyridin-7-yl]cyclopropyl)phenyl]quinazoline-4,6-diamine (200.0 mg, 0.51 mmol) in pyridine (2.0 mL) was added dropwise to the mixture at 0 C. The mixture was stirred at 0 C for another 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions: Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 33% B to 43% B in 9 min to afford N-(4-[[4-(1-[[1,2,4]triazolo[1,5-a]pyridin-7-yl]cyclopropyl)phenyl]amino]quinazolin-6-yl)but-2-ynamide (Compound 148) (31.8 mg, 13%) as a light yellow solid. LCMS (ESI, m/z): [M+H]⁺=460.1. ¹H NMR (300 MHz, DMSO-d₆): δ 10.94 (s, 1H), 9.90 (s, 1H), 8.84-8.82 (m, 1H), 8.73 (s, 1H), 8.52 (s, 1H), 8.44 (s, 1H), 7.82-7.75 (m, 4H), 7.53 (d, J=1.2 Hz, 1H), 7.37 (d, J=8.4 Hz, 2H), 7.00-6.97 (m, 1H), 2.09 (s, 3H), 1.47-1.41 (m, 4H).

### Example S39. Synthesis of N-[4-([4-[(1-cyclopropyl-2-oxopyridin-4-yl)oxy]-3-methylphenyl]amino)quinazolin-6-yl]but-2-ynamide (Compound 149)

### Step 1: Synthesis of 4-(2-methyl-4-nitrophenoxy)-1H-pyridin-2-one

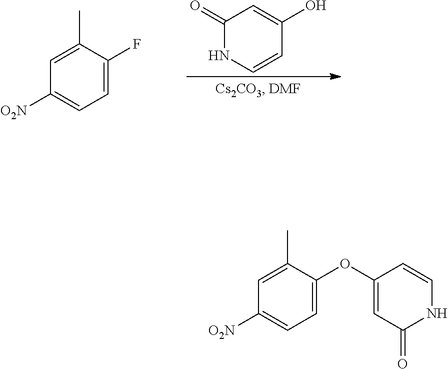 [SMILES: Cc1cc([N+](=O)[O-])ccc1F ; O=c1cc(O)cc[nH]1 ; Cc1cc([N+](=O)[O-])ccc1Oc1cc[nH]c(=O)c1]

To a solution of 1-fluoro-2-methyl-4-nitrobenzene (2.1 g, 13.54 mmol) in DMF (36.0 mL) was added 4-hydroxy-1H-pyridin-2-one (1.5 g, 13.54 mmol) and Cs₂CO₃ (8.8 g, 27.07 mmol) at room temperature under N₂. The resulting mixture was stirred at 80° C. for 16 h under N₂. After the reaction was completed, the resulting mixture was diluted with water and filtered. The solid was collected and purified by flash column chromatography with CH₂Cl₂/MeOH (10/1, v/v) to afford 4-(2-methyl-4-nitrophenoxy)-1H-pyridin-2-one (2.1 g, 63%) as a light yellow solid. LCMS (ESI, m/z): [M+H]⁺=247.1.

### Step 2: Synthesis of 1-cyclopropyl-4-(2-methyl-4-nitrophenoxy)pyridin-2-one

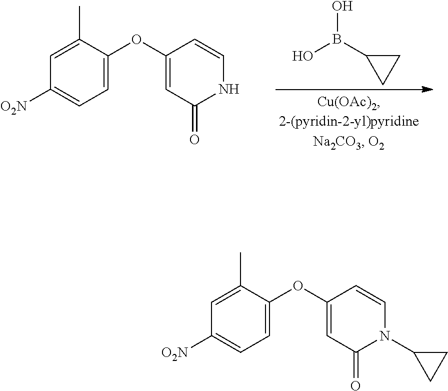 [SMILES: Cc1cc([N+](=O)[O-])ccc1Oc1cc[nH]c(=O)c1 ; OB(O)C1CC1 ; Cc1cc([N+](=O)[O-])ccc1Oc1ccn(C2CC2)c(=O)c1]

To a solution of 4-(2-methyl-4-nitrophenoxy)-1H-pyridin-2-one (500.0 mg, 2.03 mmol) in CH₂ClCH₂Cl (30.0 mL) was added cyclopropylboronic acid (453.5 mg, 5.28 mmol), Cu(OAc)₂ (394.7 mg, 2.17 mmol), Na₂CO₃ (518.7 mg, 4.89 mmol) and 2-(pyridin-2-yl)pyridine (339.4 mg, 2.17 mmol) at room temperature. The resulting mixture was stirred at 70° C. for 16 h under 02. After the reaction was completed, the resulting mixture was filtered. The filtrate was concentrated under reduced pressure. The residue was purified by flash column chromatography with CH₂Cl₂/MeOH (10/1, v/v) to afford 1-cyclopropyl-4-(2-methyl-4-nitrophenoxy)pyridin-2-one (230.0 mg, 39%) as a light yellow solid. LCMS (ESI, m/z): [M+H]⁺=287.1.

### Step 3: Synthesis of 4-(4-amino-2-methylphenoxy)-1-cyclopropylpyridin-2-one

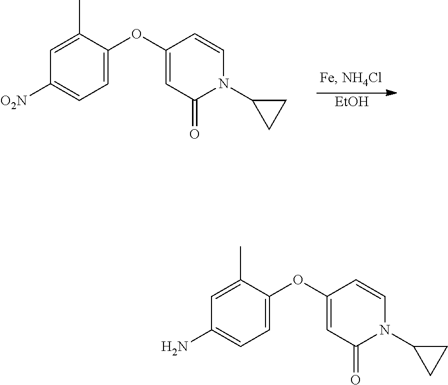 [SMILES: "EtOH" ; Cc1cc([N+](=O)[O-])ccc1Oc1ccn(C2CC2)c(=O)c1 ; Cc1cc(N)ccc1Oc1ccn(C2CC2)c(=O)c1]

To a mixture of 1-cyclopropyl-4-(2-methyl-4-nitrophenoxy)pyridin-2-one (230.0 mg, 0.80 mmol) and NH₄Cl (128.9 mg, 2.41 mmol) in EtOH (10.0 mL)/H₂O (2.0 mL) was added Fe (134.6 mg, 2.41 mmol) at 80° C. The resulting mixture was stirred at 80° C. for 1 h. After the reaction was completed, the resulting mixture was filtered. The filtrate was concentrated under reduced pressure. The residue was purified by flash column chromatography with CH₂Cl₂/MeOH (20/1, v/v) to afford 4-(4-amino-2-methylphenoxy)-1-cyclopropylpyridin-2-one (170.0 mg, 82%) as a light yellow solid. LCMS (ESI, m/z): [M+H]⁺=257.1.

### Step 4: Synthesis of 1-cyclopropyl-4-[2-methyl-4-[(6-nitroquinazolin-4-yl)amino]phenoxy]pyridin-2-one

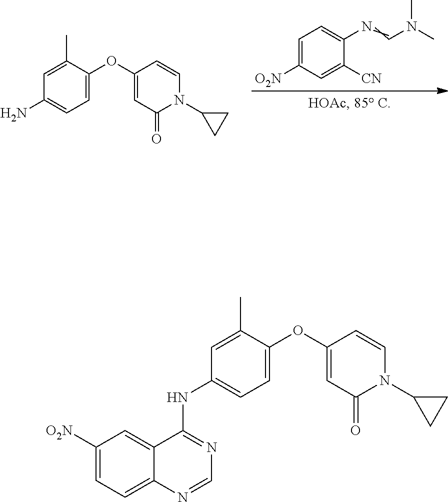 [SMILES: Cc1cc(N)ccc1Oc1ccn(C2CC2)c(=O)c1 ; CN(C)C=Nc1ccc([N+](=O)[O-])cc1C#N ; Cc1cc(Nc2ncnc3ccc([N+](=O)[O-])cc23)ccc1Oc1ccn(C2CC2)c(=O)c1]

To a mixture of 4-(4-amino-2-methylphenoxy)-1-cyclopropylpyridin-2-one (170.0 mg, 0.66 mmol) in HOAc (10.0 mL) was added N′-(2-cyano-4-nitrophenyl)-N,N-dimethylformimidamide (144.7 mg, 0.66 mmol) at room temperature. The resulting mixture was stirred at 85° C. for 1 h. After the reaction was completed, the mixture was concentrated under vacuum. The residue was purified by flash column chromatography with CH₂Cl₂/MeOH (20/1, v/v) to afford 1-cyclopropyl-4-[2-methyl-4-[(6-nitroquinazolin-4-yl)amino]phenoxy]pyridin-2-one (230.0 mg, 80%) as an orange solid. LCMS (ESI, m/z): [M+H]⁺=430.1.

### Step 5: Synthesis of 4-[4-[(6-aminoquinazolin-4-yl)amino]-2-methylphenoxy]-1-cyclopropylpyridin-2-one

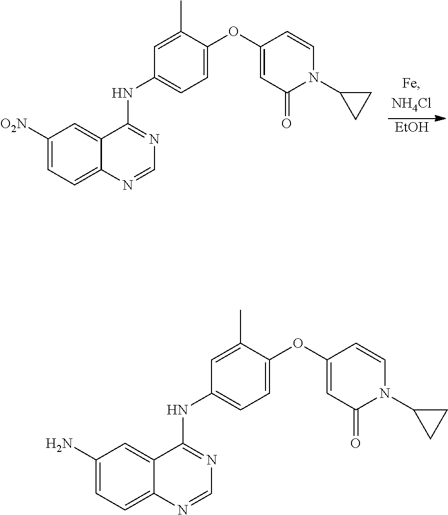 [SMILES: "EtOH" ; Cc1cc(Nc2ncnc3ccc([N+](=O)[O-])cc23)ccc1Oc1ccn(C2CC2)c(=O)c1 ; Cc1cc(Nc2ncnc3ccc(N)cc23)ccc1Oc1ccn(C2CC2)c(=O)c1]

To a mixture of 1-cyclopropyl-4-[2-methyl-4-[(6-nitroquinazolin-4-yl)amino]phenoxy]pyridin-2-one (230.0 mg, 0.54 mmol and NH₄Cl ((85.9 mg, 1.67 mmol) in EtOH (10.0 mL)/H₂O (2.0 mL) was added Fe (89.7 mg, 1.61 mmol) at 80° C. The resulting mixture was stirred at 80° C. for 3 h. After the reaction was completed, the resulting mixture was filtered. The filtrate was concentrated under reduced pressure. The mixture was purified by flash column chromatography with CH₂Cl₂/MeOH (10/1, v/v) to afford 4-[4-[(6-aminoquinazolin-4-yl)amino]-2-methylphenoxy]-1-cyclopropylpyridin-2-one (200.0 mg, 93%) as a light yellow solid. LCMS (ESI, m/z): [M+H]⁺=400.2.

### Step 6: Synthesis of N-[4-([4-[(1-cyclopropyl-2-oxopyridin-4-yl)oxy]-3-methylphenyl]amino)quinazolin-6-yl]but-2-ynamide (Compound 149)

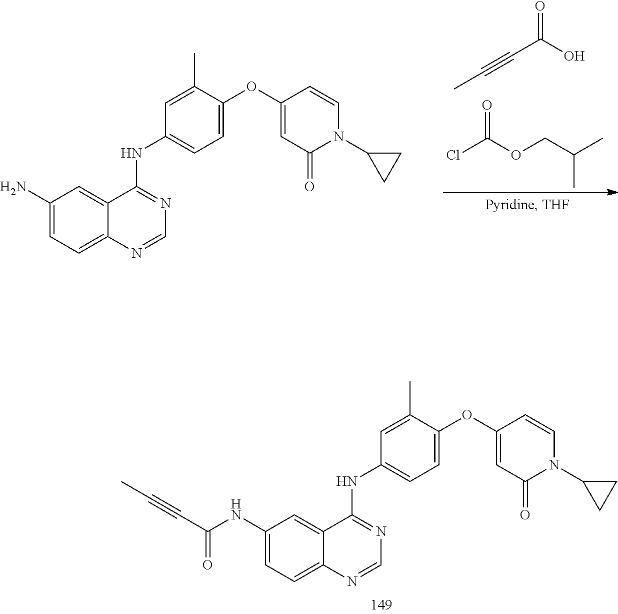

To a solution of 2-butynoic acid (183.9 mg, 2.188 mmol) in THF (10.0 mL) was added 2-methylpropyl carbonochloridate (402.1 mg, 2.95 mmol) at 0 C. The resulting mixture was stirred at 0 C for 5 min. Then N-Methylmorpholine (510.5 mg, 5.05 mmol) was added dropwise to the mixture at 0 C. The resulting mixture was stirred at 0 C for another 10 min. Then a solution of 4-[4-[(6-aminoquinazolin-4-yl)amino]-2-methylphenoxy]-1-cyclopropylpyridin-2-one (200.0 mg, 0.50 mmol) in pyridine (2.0 mL) was added dropwise to the mixture at 0 C. The mixture was stirred at 0 C for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions: Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 20% B to 46% B in 10 min, 254/220 nm; RT1: 9.37 min to afford N-[4-([4-[(1-cyclopropyl-2-oxopyridin-4-yl)oxy]-3-methylphenyl]amino)quinazolin-6-yl]but-2-ynamide (Compound 149) (54.3 mg, 23%) as a white solid. LCMS (ESI, m/z): [M+H]⁺=466.2. ¹H NMR (300 MHz, DMSO-d₆): δ 10.94 (s, 1H), 9.88 (s, 1H), 8.75 (s, 1H), 8.55 (s, 1H), 7.82-7.72 (m, 4H), 7.57 (d, J=7.8 Hz, 1H), 7.11 (d, J=8.7 Hz, 1H), 6.06-6.03 (m, 1H), 5.32 (d, J=2.7 Hz, 1H), 3.27-3.20 (m, 1H), 2.15 (s, 3H), 2.10 (s, 3H), 0.99-0.92 (m, 2H), 0.90-0.87 (m, 2H).

### Example S40. Synthesis of N-[7-[2-(dimethylamino)ethoxy]-4-[(3-methyl-4-[[1,2,4]triazolo[1,5-a]pyridin-7-yloxy]phenyl)amino]quinazolin-6-yl]but-2-ynamide (Compound 150)

### Step 1: Synthesis of 7-[2-(dimethylamino)ethoxy]-6-nitro-3H-quinazolin-4-one

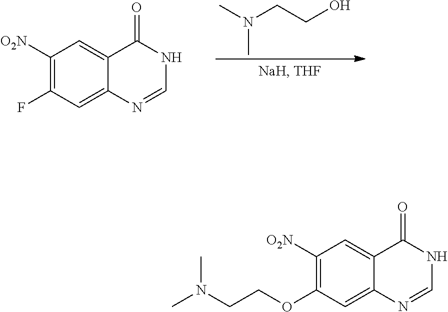

To a solution of dimethylaminoethanol (6.4 g, 71.72 mmol) in THF (150.0 mL) was added NaH (3.8 g, 60%) at 0° C. under N₂. The mixture was stirred at 0° C. for 30 min. Then 7-fluoro-6-nitro-3H-quinazolin-4-one (5.0 g, 23.91 mmol) was added to the mixture at 0° C. under N₂, the mixture was allowed to warm to room temperature and stirred for 1 h. After the reaction was completed, the resulting mixture was diluted with H₂O and extracted with DCM. The combined organic layer was washed with brine, dried over anhydrous sodium sulfate and filtered. The filtrate was concentrated under reduced pressure. The residue was purified by flash column chromatography with CH₂Cl₂/MeOH (5/1, v/v) to afford 7-[2-(dimethylamino)ethoxy]-6-nitro-3H-quinazolin-4-one (4.9 g, 73%) as a yellow solid. LCMS (ESI, m/z): [M+H]⁺=279.1.

### Step 2: Synthesis of [2-[(4-chloro-6-nitroquinazolin-7-yl)oxy]ethyl]dimethylamine)

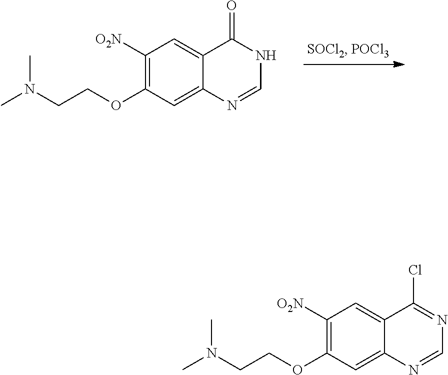 [SMILES: CN(C)CCOc1cc2nc[nH]c(=O)c2cc1[N+](=O)[O-] ; CN(C)CCOc1cc2ncnc(Cl)c2cc1[N+](=O)[O-]]

To a solution of 7-[2-(dimethylamino)ethoxy]-6-nitro-3H-quinazolin-4-one (1.0 g, 3.59 mmol) in SOCl₂ (10.0 mL) were added POCl₃ (1.0 mL) and DMF (0.1 mL) at room temperature. The resulting mixture was stirred at 85° C. for 2 h. After the reaction was completed, the mixture was concentrated under vacuum to afford [2-[(4-chloro-6-nitroquinazolin-7-yl)oxy]ethyl]dimethylamine (1.0 g, crude) as a light yellow solid. LCMS (ESI, m/z): [M+H]⁺=297.1.

### Step 3: Synthesis of 7-[2-(dimethylamino)ethoxy]-N-(3-methyl-4-[[1,2,4]triazolo[1,5-a]pyridin-7-yloxy]phenyl)-6-nitroquinazolin-4-amine

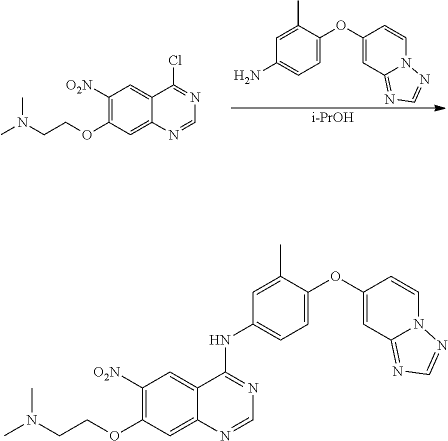 [SMILES: "i-PrOH" ; CN(C)CCOc1cc2ncnc(Cl)c2cc1[N+](=O)[O-] ; Cc1cc(N)ccc1Oc1ccn2ncnc2c1 ; Cc1cc(Nc2ncnc3cc(OCCN(C)C)c([N+](=O)[O-])cc23)ccc1Oc1ccn2ncnc2c1]

To a mixture of [2-[(4-chloro-6-nitroquinazolin-7-yl)oxy]ethyl]dimethylamine (1.0 g, crude) in i-PrOH (30.0 mL) was added 3-methyl-4-[[1,2,4]triazolo[1,5-a]pyridin-7-yloxy]aniline (0.8 g, 3.37 mmol) at room temperature. The resulting mixture was stirred at room temperature for 2 h. After the reaction was completed, the mixture was concentrated under vacuum. The residue was purified by flash column chromatography with CH₂Cl₂/MeOH (7/3, v/v) to afford 7-[2-(dimethylamino)ethoxy]-N-(3-methyl-4-[[1,2,4]triazolo[1,5-a]pyridin-7-yloxy]phenyl)-6-nitroquinazolin-4-amine (2.0 g, 98%) as a yellow solid. LCMS (ESI, m/z): [M+H]⁺=501.2.

### Step 4: Synthesis of 7-[2-(dimethylamino)ethoxy]-N4-(3-methyl-4-[[1,2,4]triazolo[1,5-a]pyridin-7-yloxy]phenyl)quinazoline-4,6-diamine

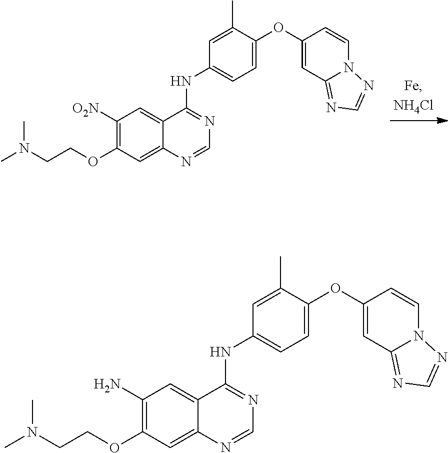 [SMILES: Cc1cc(Nc2ncnc3cc(OCCN(C)C)c([N+](=O)[O-])cc23)ccc1Oc1ccn2ncnc2c1 ; Cc1cc(Nc2ncnc3cc(OCCN(C)C)c(N)cc23)ccc1Oc1ccn2ncnc2c1]

To a mixture of 7-[2-(dimethylamino)ethoxy]-N-(3-methyl-4-[[1,2,4]triazolo[1,5-a]pyridin-7-yloxy]phenyl)-6-nitroquinazolin-4-amine (2.0 g, 3.99 mmol) and NH₄Cl (0.85 g, 15.98 mmol) in EtOH (24.0 mL)/H₂O (6.0 mL) was added Fe (0.7 g, 11.99 mmol) at 80° C. The resulting mixture was stirred at 80° C. for 3 h. After the reaction was completed, the resulting mixture was filtered. The filtrate was concentrated under reduced pressure. The mixture was purified by flash column chromatography with petroleum ether/EtOAc (2/1, v/v) to afford 7-[2-(dimethylamino)ethoxy]-N4-(3-methyl-4-[[1,2,4]triazolo[1,5-a]pyridin-7-yloxy]phenyl)quinazoline-4,6-diamine (495.0 mg, 26%) as a light yellow solid. LCMS (ESI, m/z): [M+H]⁺=471.2.

### Step 5: Synthesis of N-[7-[2-(dimethylamino)ethoxy]-4-[(3-methyl-4-[[1,2,4]triazolo[1,5-a]pyridin-7-yloxy]phenyl)amino]quinazolin-6-yl]but-2-ynamide (Compound 150)

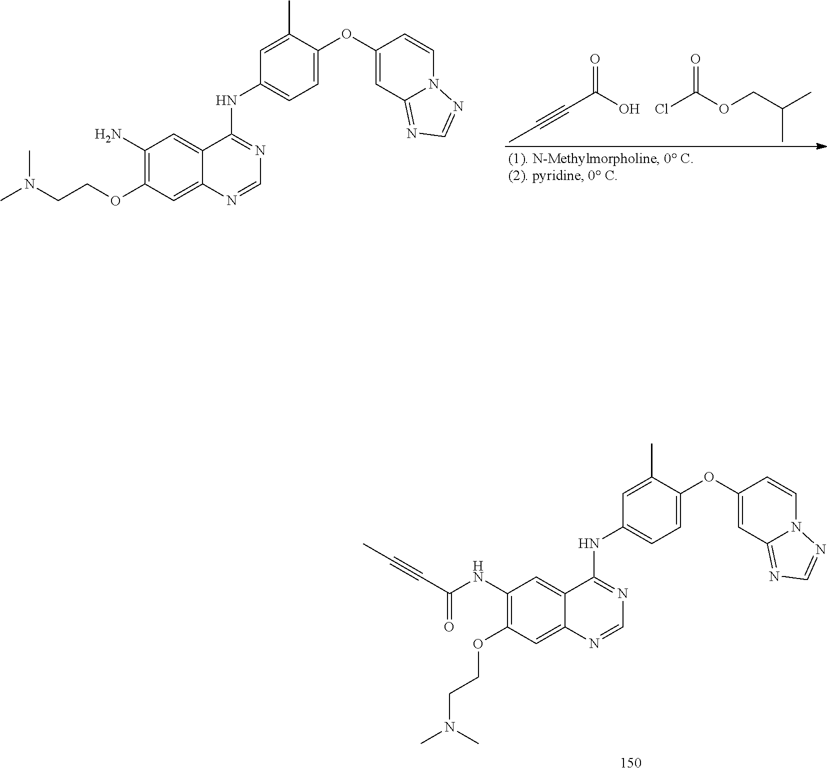

To a solution of 2-butynoic acid (117.1 mg, 1.39 mmol) in THF (10.0 mL) was added 2-methylpropyl carbonochloridate (255.9 mg, 1.87 mmol) at room temperature under N2. The resulting mixture was stirred at 0° C. for 5 min. Then N-Methylmorpholine (325.0 mg, 3.21 mmol) was added dropwise to the mixture at 0° C. The mixture was stirred at 0° C. for 10 min. Then a solution of 7-[2-(dimethylamino)ethoxy]-N4-(3-methyl-4-[[1,2,4]triazolo[1,5-a]pyridin-7-yloxy]phenyl)quinazoline-4,6-diamine (150.0 mg, 0.32 mmol) in pyridine (2.0 mL) was added dto the mixture at 0° C. The mixture was stirred at 0° C. for another 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions: Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 37% B to 47% B in 7 min, 254/220 nm; RT1:6.88 min to afford N-[7-[2-(dimethylamino)ethoxy]-4-[(3-methyl-4-[[1,2,4]triazolo[1,5-a]pyridin-7-yloxy]phenyl)amino]quinazolin-6-yl]but-2-ynamide (Compound 150) (26.6 mg, 15%) as a white solid. LCMS (ESI, m/z): [M+H]⁺=537.3. ¹H NMR (300 MHz, DMSO-d₆): δ 10.24 (s, 1H), 9.76 (s, 1H), 8.94 (d, J=7.2 Hz, 1H), 8.73 (s, 1H), 8.54 (s, 1H), 8.39 (s, 1H), 7.83-7.79 (m, 2H), 7.35 (s, 1H), 7.20 (d, J=8.7 Hz, 1H), 7.05-7.02 (m, 1H), 6.80 (d, J=2.4 Hz, 1H), 4.31-4.28 (m, 2H), 2.74-2.70 (m, 2H), 2.29 (s, 6H), 2.19 (s, 3H), 2.08 (s, 3H).

### Example S41. Synthesis of 5-(dimethylamino)-N-[4-[(3-methyl-4-[[1,2,4]triazolo[1,5-a]pyridin-7-yloxy]phenyl)amino]quinazolin-6-yl]pent-2-ynamide (Compound 151)

### Step 1: Synthesis of tert-butyl N-(but-3-yn-1-yl)-N-methylcarbamate

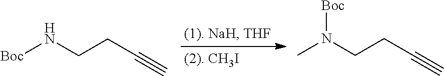

To a solution of tert-butyl N-(but-3-yn-1-yl)carbamate (5.0 g, 29.72 mmol) in THF (100.0 mL) was added NaH (1.6 g, 60%) at 0° C. under N₂. The mixture was stirred at 0° C. for 1 h. Then CH₃I (7.7 g, 54.24 mmol) was added to the mixture at 0° C. The mixture was stirred at room temperature for 16 h. After the reaction was completed, the mixture was quenched with saturated NH₄Cl solution and then extracted with ethyl acetate. The combined organic layer was washed with brine, dried over anhydrous sodium sulfate and filtered. The filtrate was concentrated under vacuum. The residue was purified by flash column chromatography with petroleum ether/EtOAc (4/1, v/v) to afford tert-butyl N-(but-3-yn-1-yl)-N-methylcarbamate (4.7 g, 86%) as a light yellow oil. LCMS (ESI, m/z): [M+H]⁺=184.1.

### Step 2: Synthesis of propyl 5-[(tert-butoxycarbonyl)(methyl)amino]pent-2-ynoate

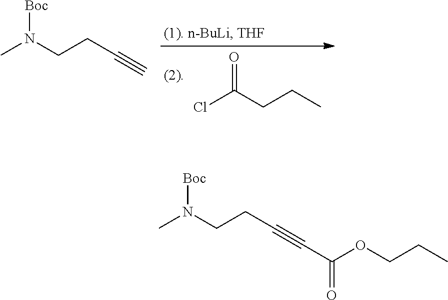

To a solution of tert-butyl N-(but-3-yn-1-yl)-N-methylcarbamate (2.1 g, 11.46 mmol) in THF (30.0 mL) was added dropwise n-BuLi in hexanes (5.5 mL, 2.5 mol/L) at −78° C. under N₂. The mixture was stirred at −78° C. for 1 h. Then butyryl chloride (2.1 g, 19.70 mmol) was added dropwise to the mixture at −78° C. The mixture was stirred at −78° C. for another 2 h. After the reaction was completed, the reaction mixture was quenched with saturated NH₄Cl solution and then extracted with ethyl acetate. The combined organic layer was washed with brine, dried over anhydrous sodium sulfate and filtered. The filtrate was concentrated under vacuum. The residue was purified by flash column chromatography with petroleum ether/EtOAc (10/1, v/v) to afford propyl 5-[(tert-butoxycarbonyl)(methyl)amino]pent-2-ynoate (2.0 g, 64%) as a colorless oil. LCMS (ESI, m/z): [M+H]⁺=270.2.

### Step 3: Synthesis of tert-butyl N-methyl-N-[4-([4-[(3-methyl-4-[[1,2,4]triazolo[1,5-a]pyridin-7-yloxy]phenyl)amino]quinazolin-6-yl]carbamoyl)but-3-yn-1-yl]carbamate

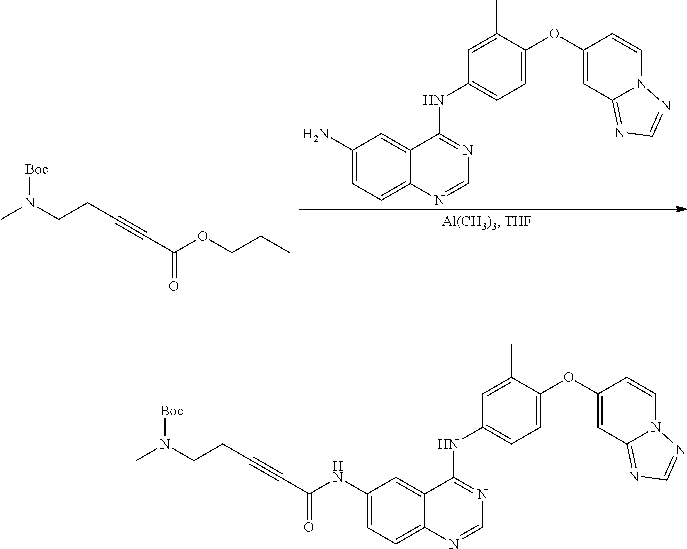 [SMILES: CCCOC(=O)C#CCCN(C)C(=O)OC(C)(C)C ; Cc1cc(Nc2ncnc3ccc(N)cc23)ccc1Oc1ccn2ncnc2c1 ; Cc1cc(Nc2ncnc3ccc(NC(=O)C#CCCN(C)C(=O)OC(C)(C)C)cc23)ccc1Oc1ccn2ncnc2c1]

To a mixture of propyl 5-[(tert-butoxycarbonyl)(methyl)amino]pent-2-ynoate (1.0 g, 3.71 mmol) and N4-(3-methyl-4-[[1,2,4]triazolo[1,5-a]pyridin-7-yloxy]phenyl)quinazoline-4,6-diamine (2.0 g, 5.21 mmol) in THF (20.0 mL) was added dropwise AlMe₃ (8.0 mL, 2 mol/L) at 0° C. The mixture was stirred at 80° C. for 3 h. After the reaction was completed, the mixture was quenched with NH₄Cl solution and extracted with ethyl acetate. The combined organic layer was washed with brine, dried over anhydrous sodium sulfate and filtered. The filtrate was concentrated under vacuum. The residue was purified by reverse phase flash column chromatography to afford CH₃CN/H₂O (70/30, v/v) to afford tert-butyl N-methyl-N-[4-([4-[(3-methyl-4-[[1,2,4]triazolo[1,5-a]pyridin-7-yloxy]phenyl)amino]quinazolin-6-yl]carbamoyl)but-3-yn-1-yl]carbamate (700.0 mg, 31%) as a yellow solid. LCMS (ESI, m/z): [M+H]⁺=593.3.

### Step 4: Synthesis of N-[4-[(3-methyl-4-[[1,2,4]triazolo[1,5-a]pyridin-7-yloxy]phenyl)amino]quinazolin-6-yl]-5-(methylamino)pent-2-ynamide hydrochloride

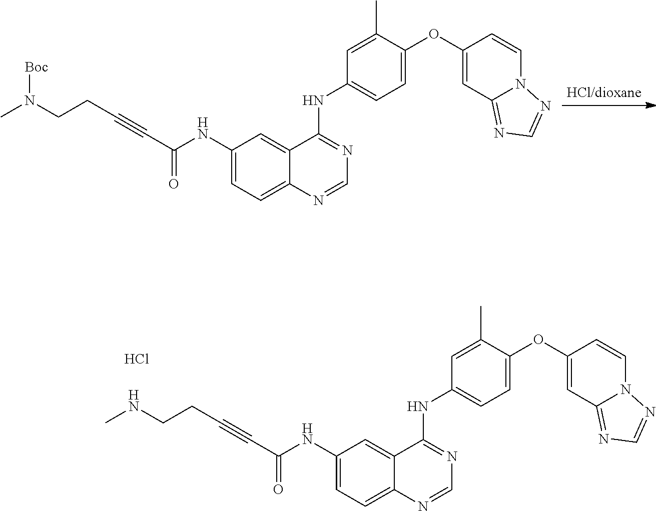 [SMILES: Cc1cc(Nc2ncnc3ccc(NC(=O)C#CCCN(C)C(=O)OC(C)(C)C)cc23)ccc1Oc1ccn2ncnc2c1 ; CNCCC#CC(=O)Nc1ccc2ncnc(Nc3ccc(Oc4ccn5ncnc5c4)c(C)c3)c2c1 ; Cl]

To a solution of tert-butyl N-methyl-N-[4-([4-[(3-methyl-4-[[1,2,4]triazolo[1,5-a]pyridin-7-yloxy]phenyl)amino]quinazolin-6-yl]carbamoyl)but-3-yn-1-yl]carbamate (550.0 mg, 0.92 mmol) in Et₂O (5.0 mL, 48.19 mmol) was added HCl/1,4-dioxane (10.0 mL, 4 mol/L). The mixture was stirred at room temperature for 4 h. After the reaction was completed, the mixture was evaporated in vacuo to afford N-[4-[(3-methyl-4-[[1,2,4]triazolo[1,5-a]pyridin-7-yloxy]phenyl)amino]quinazolin-6-yl]-5-(methylamino)pent-2-ynamide hydrochloride (510.0 mg, crude) as a yellow solid. LCMS (ESI, m/z): [M+H]⁺=493.3.

### Step 5: Synthesis of 5-(dimethylamino)-N-[4-[(3-methyl-4-[[1,2,4]triazolo[1,5-a]pyridin-7-yloxy]phenyl)amino]quinazolin-6-yl]pent-2-ynamide (Compound 151)

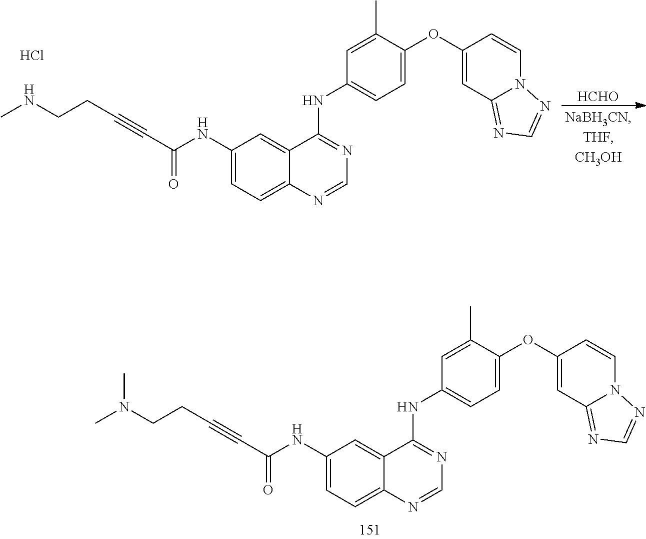

To a mixture of N-[4-[(3-methyl-4-[[1,2,4]triazolo[1,5-a]pyridin-7-yloxy]phenyl)amino]quinazolin-6-yl]-5-(methylamino)pent-2-ynamide hydrochloride (130.0 mg, crude) and HCHO (83.0 mg, 0.82 mmol, 30%) in CH₃OH (1.0 mL) and THF (6.0 mL) was added NaBH₃CN (74.0 mg, 1.17 mmol). The mixture was stirred at room temperature for 2 h. After the reaction was completed, the mixture evaporated in vacuo. The residue was purified by flash column chromatography with CH₂Cl₂/CH₃OH (82/18, v/v) and then purified by Prep-HPLC with the following conditions: (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 31% B to 41% B in 8 min 254/220 nm) to afford 5-(dimethylamino)-N-[4-[(3-methyl-4-[[1,2,4]triazolo[1,5-a]pyridin-7-yloxy]phenyl)amino]quinazolin-6-yl]pent-2-ynamide (Compound 151) (10.2 mg, 7%) was a white solid. LCMS (ESI, m/z): [M+H]⁺=507.2. ¹H NMR (300 MHz, DMSO-d₆): δ 10.95 (s, 1H), 9.89 (s, 1H), 8.94 (d, J=7.5 Hz, 1H), 8.75 (s, 1H), 8.56 (s, 1H), 8.39 (s, 1H), 7.83-7.76 (m, 4H), 7.21 (d, J=8.7 Hz, 1H), 7.06-7.02 (m, 1H), 6.80 (d, J=2.4 Hz, 1H), 2.60-2.51 (m, 3H), 2.20-2.19 (m, 9H).

### Example S42. Synthesis of N-[4-[(3-chloro-4-[[1,2,4]triazolo[1,5-a]pyridin-7-ylmethyl]phenyl)amino]quinazolin-6-yl]-5-hydroxypent-2-ynamide (Compound 152)

### Step 1: Synthesis of methyl 2-[(E)-[(hydroxyamino)methylidene]amino]pyridine-4-carboxylate

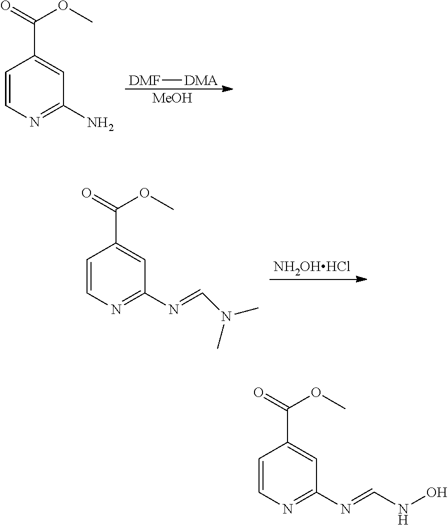

To a solution of methyl 2-aminoisonicotinate (20.0 g, 131.44 mmol) in CH₃OH (200.0 mL) was added DMF-DMA (23.5 g, 197.21 mmol) at room temperature. The resulting mixture was stirred at 75° C. for 3 h. Then hydroxylamine hydrochloride (25.8 g, 371.55 mmol) was added to the mixture at room temperature. The resulting mixture was stirred at 50° C. for another 1 h. After the reaction was completed, the reaction mixture was cooled to room temperature and filtered. The solid was washed with CH₃OH and dried to afford methyl 2-[(E)-[(hydroxyamino)methylidene]amino]pyridine-4-carboxylate (18.9 g, crude) as a white solid. LCMS (ESI, m/z): [M+H]⁺=196.0.

### Step 2: Synthesis of methyl [1,2,4]triazolo[1,5-a]pyridine-7-carboxylate

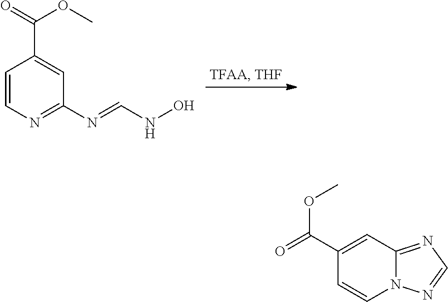

To a solution of methyl 2-[(E)-[(hydroxyamino)methylidene]amino]pyridine-4-carboxylate (18.9 g, crude) in THF (200.0 mL) was added TFAA (22.3 g, 106.50 mmol) at room temperature. The resulting mixture was stirred at room temperature for 5 h. After the reaction was completed, the pH value of the mixture was adjusted to 7 with saturated NaHCO₃(aq.). The resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford methyl [1,2,4]triazolo[1,5-a]pyridine-7-carboxylate (13.7 g, 79%) as a white solid. LCMS (ESI, m/z): [M+H]⁺=178.1.

### Step 3: Synthesis of [1,2,4]triazolo[1,5-a]pyridin-7-ylmethanol

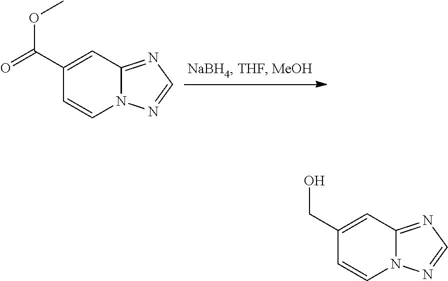

To a solution of methyl [1,2,4]triazolo[1,5-a]pyridine-7-carboxylate (13.7 g, 77.33 mmol) in THF (150.0 mL)/CH₃OH (15 mL) was added NaBH₄ (14.6 g, 386.65 mmol) at 0° C. under N₂. The resulting mixture was stirred at room temperature for 4 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (9/1, v/v) to afford [1,2,4]triazolo[1,5-a]pyridin-7-ylmethanol (4.8 g, 41%) as a white solid. LCMS (ESI, m/z): [M+H]⁺=150.1.

### Step 4: of 7-(bromomethyl)-[1,2,4]triazolo[1,5-a]pyridine

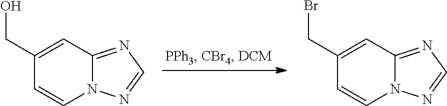 [SMILES: OCc1ccn2ncnc2c1 ; BrCc1ccn2ncnc2c1]

To a mixture of [1,2,4]triazolo[1,5-a]pyridin-7-ylmethanol (2.5 g, 16.76 mmol) and PPh₃ (6.5 g, 25.14 mmol) in CH₂Cl₂ (30.0 mL) was added CBr₄ (8.3 g, 25.14 mmol) at 0° C. under N₂. The resulting mixture was stirred at room temperature for 3 h. After the reaction was completed, the mixture was concentrated under reduced pressure. The residue was purified by flash column chromatography with CH₂Cl₂/CH₃OH (10/1, v/v) to afford 7-(bromomethyl)-[1,2,4]triazolo[1,5-a]pyridine (1.5 g, 42%) as a white solid. LCMS (ESI, m/z): [M+H]⁺=212.0.

### Step 5: Synthesis of tert-butyl 4-([1,2,4]triazolo[1,5-a]pyridin-7-ylmethyl)-3-chlorophenylcarbamate

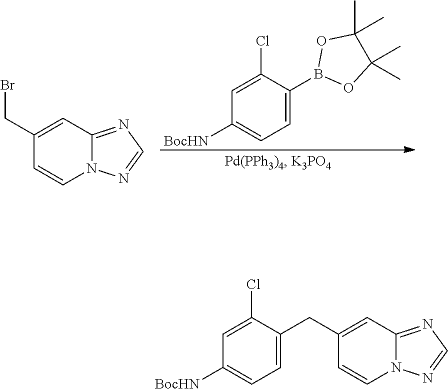 [SMILES: BrCc1ccn2ncnc2c1 ; CC(C)(C)OC(=O)Nc1ccc(B2OC(C)(C)C(C)(C)O2)c(Cl)c1 ; CC(C)(C)OC(=O)Nc1ccc(Cc2ccn3ncnc3c2)c(Cl)c1]

To a mixture of tert-butyl N-[3-chloro-4-(4,4,5,5-tetramethyl-1,3,2-dioxaborolan-2-yl)phenyl]carbamate (2.0 g, 5.64 mmol) and 7-(bromomethyl)-[1,2,4]triazolo[1,5-a]pyridine (1.2 g, 5.64 mmol) in dioxane (20.0 mL)/H₂O (2.0 mL) was added K₃PO₄ (3.6 g, 16.96 mmol) and Pd(PPh₃)₄ (1.2 g, 1.12 mmol) at room temperature under N₂. The resulting mixture was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/8, v/v) to afford tert-butyl 4-([1,2,4]triazolo[1,5-a]pyridin-7-ylmethyl)-3-chlorophenylcarbamate (2.0 g, 98%) as a light yellow solid. LCMS (ESI, m/z): [M+H]⁺=359.1.

### Step 6: Synthesis of 4-([1,2,4]triazolo[1,5-a]pyridin-7-ylmethyl)-3-chlorobenzenamine

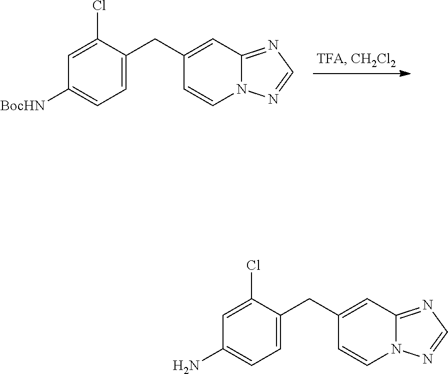 [SMILES: CC(C)(C)OC(=O)Nc1ccc(Cc2ccn3ncnc3c2)c(Cl)c1 ; Nc1ccc(Cc2ccn3ncnc3c2)c(Cl)c1]

To a solution of tert-butyl 4-([1,2,4]triazolo[1,5-a]pyridin-7-ylmethyl)-3-chlorophenylcarbamate (2.0 g, 5.59 mmol) in CH₂Cl₂ (10.0 mL) was added TFA (10.0 mL) at room temperature. The resulting mixture was stirred at room temperature for 1 h. After the reaction was completed, the pH value of the mixture was adjusted to 5 with saturated NaHCO₃(aq.). The resulting mixture was diluted with H₂O and extracted with CH₂Cl₂. The combined organic layer was washed with brine, dried over anhydrous sodium sulfate and filtered. The filtrate was concentrated under reduced pressure to afford 4-([1,2,4]triazolo[1,5-a]pyridin-7-ylmethyl)-3-chlorobenzenamine (1.0 g, crude) as a yellow solid. LCMS (ESI, m/z): [M+H]⁺=259.1.

### Step 7: Synthesis of N-(3-chloro-4-[[1,2,4]triazolo[1,5-a]pyridin-7-ylmethyl]phenyl)-6-nitroquinazolin-4-amine

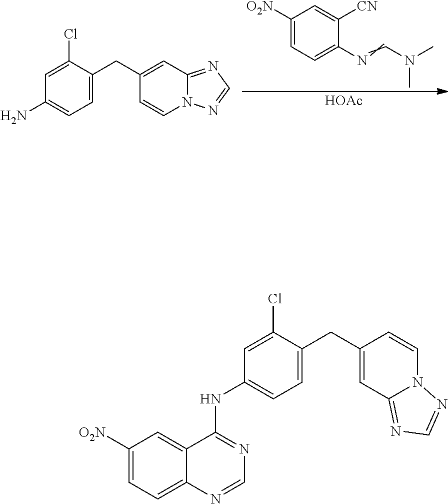 [SMILES: Nc1ccc(Cc2ccn3ncnc3c2)c(Cl)c1 ; CN(C)C=Nc1ccc([N+](=O)[O-])cc1C#N ; O=[N+]([O-])c1ccc2ncnc(Nc3ccc(Cc4ccn5ncnc5c4)c(Cl)c3)c2c1 ; "HOAc"]

To a solution of 4-([1,2,4]triazolo[1,5-a]pyridin-7-ylmethyl)-3-chlorobenzenamine (1.0 g, 3.88 mmol) in HOAc (8.0 mL) was added N′-(2-cyano-4-nitrophenyl)-N,N-dimethylformimidamide (1.01 g, 7.76 mmol) at room temperature. The resulting mixture was stirred at 85° C. for 2 h. After the reaction was completed, the resulting mixture was concentrated under vacuum. The residue was purified by flash column chromatography with CH₂Cl₂/CH₃OH (9/1, v/v) to afford N-(3-chloro-4-[[1,2,4]triazolo[1,5-a]pyridin-7-ylmethyl]phenyl)-6-nitroquinazolin-4-amine (720.0 mg, 59%) as a yellow solid. LCMS (ESI, m/z): [M+H]⁺=432.0.

### Step 8: Synthesis of N4-(3-chloro-4-[[1,2,4]triazolo[1,5-a]pyridin-7-ylmethyl]phenyl)quinazoline-4,6-diamine

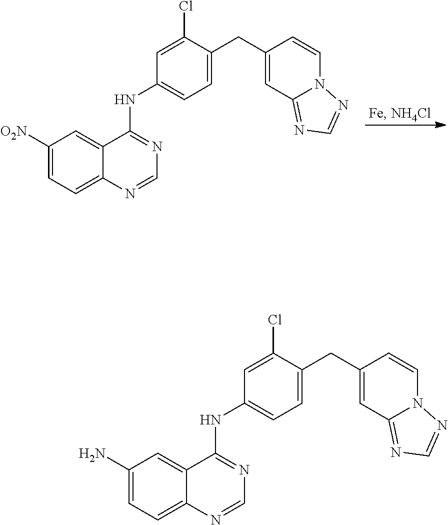

To a mixture of N-(3-chloro-4-[[1,2,4]triazolo[1,5-a]pyridin-7-ylmethyl]phenyl)-6-nitroquinazolin-4-amine (720.0 mg, 1.67 mmol) and NH₄Cl (893.3 mg, 16.7 mmol) in CH₃OH (10.0 mL)/H₂O (2.0 mL) was added Fe (935.2 mg, 16.7 mmol) at room temperature. The resulting mixture was stirred at 80° C. for 1 h. After the reaction was completed, the resulting mixture was concentrated under vacuum. The residue was purified by flash column chromatography with CH₂Cl₂/CH₃OH (9/1, v/v) to afford N4-(3-chloro-4-[[1,2,4]triazolo[1,5-a]pyridin-7-ylmethyl]phenyl)quinazoline-4,6-diamine (440.0 mg, 65%) as a yellow solid. LCMS (ESI, m/z): [M+H]⁺=402.1.

### Step 9: Synthesis of N-[4-[(3-chloro-4-[[1,2,4]triazolo[1,5-a]pyridin-7-ylmethyl]phenyl)amino]quinazolin-6-yl]-5-(oxan-2-yloxy)pent-2-ynamide

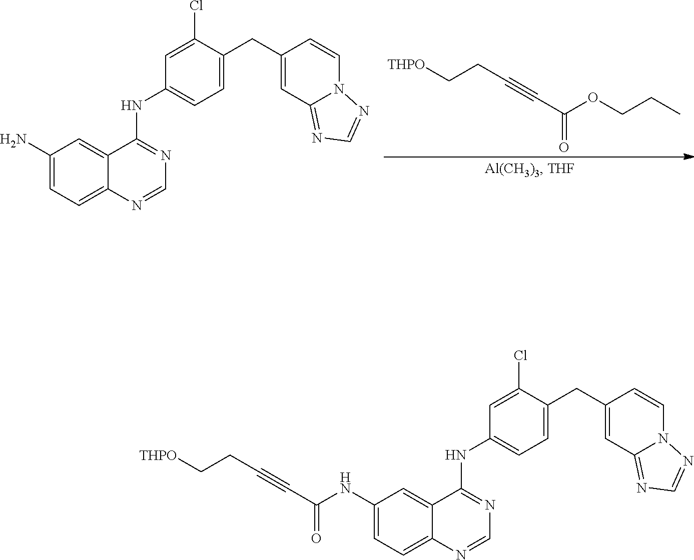

To a mixture of N4-(3-chloro-4-[[1,2,4]triazolo[1,5-a]pyridin-7-ylmethyl]phenyl)quinazoline-4,6-diamine (300.0 mg, 0.74 mmol) and propyl 5-(oxan-2-yloxy)pent-2-ynoate (538.1 mg, 2.24 mmol) in THF (10.0 mL) was added dropwise trimethylaluminium (1.95 mL, 2 mol/L) at 0° C. The resulting mixture was stirred at 80° C. for 3 h. After the reaction was completed, the reaction mixture was quenched with sat. NH₄Cl (aq.) at room temperature. The resulting mixture was diluted with H₂O and extracted with CH₂Cl₂. The combined organic layer was washed with brine, dried over anhydrous sodium sulfate and filtered. The filtrate was concentrated under reduced pressure. The residue was purified by flash column chromatography with CH₂Cl₂/CH₃OH (9/1, v/v) to afford N-[4-[(3-chloro-4-[[1,2,4]triazolo[1,5-a]pyridin-7-ylmethyl]phenyl)amino]quinazolin-6-yl]-5-(oxan-2-yloxy)pent-2-ynamide (110.0 mg, 25%) as a yellow solid. LCMS (ESI, m/z): [M+H]⁺=582.2.

### Step 10: Synthesis of N-[4-[(3-chloro-4-[[1,2,4]triazolo[1,5-a]pyridin-7-ylmethyl]phenyl)amino]quinazolin-6-yl]-5-hydroxypent-2-ynamide (Compound 152)

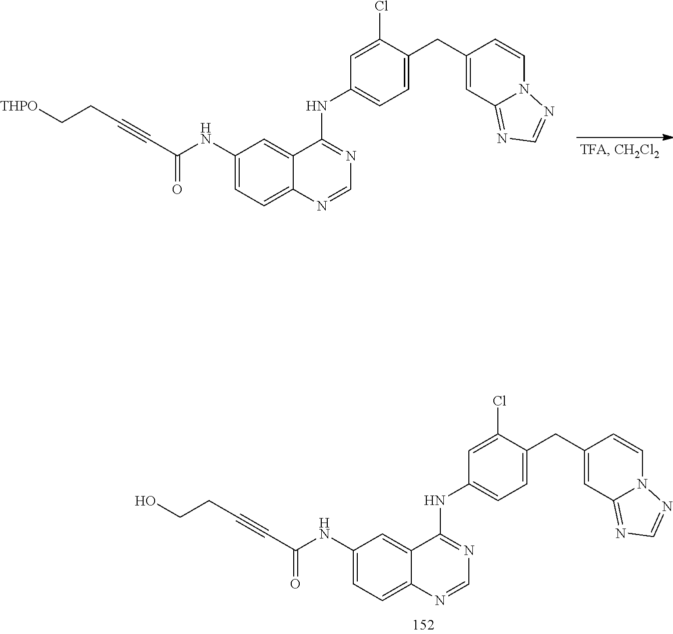

A mixture of N-[4-[(3-chloro-4-[[1,2,4]triazolo[1,5-a]pyridin-7-ylmethyl]phenyl)amino]quinazolin-6-yl]-5-(oxan-2-yloxy)pent-2-ynamide (100.0 mg, 0.17 mmol) and TFA (2.0 mL) in CH₂Cl₂ (2.0 mL) was stirred at room temperature for 2 h. After the reaction was completed, the resulting mixture was concentrated under vacuum. The residue was purified by reverse phase flash chromatography with H₂O/ACN (1/1, v/v) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 16% B to 43% B in 9 min, 254/220 nm) to afford N-[4-[(3-chloro-4-[[1,2,4]triazolo[1,5-a]pyridin-7-ylmethyl]phenyl)amino]quinazolin-6-yl]-5-hydroxypent-2-ynamide (Compound 152) (13.5 mg, 15%) as a white solid. LCMS (ESI, m/z): [M+H]⁺=498.2. ¹H NMR (300 MHz, DMSO-d₆): δ 10.93 (s, 1H), 10.01 (s, 1H), 8.87 (d, J=6.9 Hz, 1H), 8.72 (s, 1H), 8.58 (s, 1H), 8.43 (s, 1H), 8.05 (s, 1H), 7.84-7.75 (m, 3H), 7.57 (d, J=0.9 Hz, 1H), 7.45 (d, J=8.4 Hz, 1H), 7.07-7.04 (m, 1H), 5.05-4.95 (m, 1H), 4.21 (s, 2H), 3.65-3.54 (m, 2H), 2.72-2.60 (m, 2H).

### Example S43. Synthesis of N-[4-[(3-fluoro-4-[[1,2,4]triazolo[1,5-a]pyridin-7-ylmethyl]phenyl)amino]quinazolin-6-yl]-5-hydroxypent-2-ynamide (Compound 153)

### Step 1: Synthesis of tert-butyl N-[3-fluoro-4-(4,4,5,5-tetramethyl-1,3,2-dioxaborolan-2-yl)phenyl]carbamate

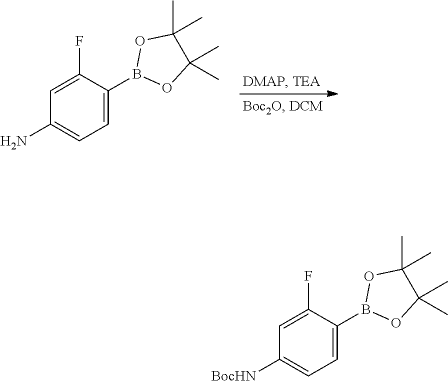 [SMILES: CC1(C)OB(c2ccc(N)cc2F)OC1(C)C ; CC(C)(C)OC(=O)Nc1ccc(B2OC(C)(C)C(C)(C)O2)c(F)c1]

To a solution of 3-fluoro-4-(4,4,5,5-tetramethyl-1,3,2-dioxaborolan-2-yl)aniline (5.0 g, 21.09 mmol) in DCM (50.0 mL) were added TEA (4.2 g, 42.18 mmol), DMAP (51.5 mg, 0.42 mmol) and Boc₂O (4.6 g, 21.09 mmol) at room temperature. The resulting mixture was stirred at room temperature for 16 h. After the reaction was completed, the resulting mixture was concentrated under reduced pressure. The residue was purified by flash column chromatography with petroleum ether/ethyl acetate (8/2, v/v) to afford tert-butyl N-[3-fluoro-4-(4,4,5,5-tetramethyl-1,3,2-dioxaborolan-2-yl)phenyl]carbamate (1.4 g, 19%) as a yellow solid. LCMS (ESI, m/z): [M+H]⁺=338.0.

### Step 2: Synthesis of tert-butyl N-(3-fluoro-4-[[1,2,4]triazolo[1,5-a]pyridin-7-ylmethyl]phenyl)carbamate

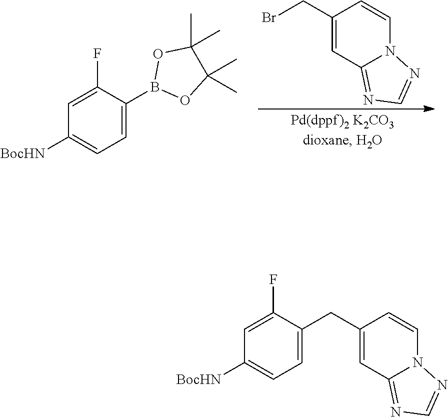 [SMILES: CC(C)(C)OC(=O)Nc1ccc(B2OC(C)(C)C(C)(C)O2)c(F)c1 ; BrCc1ccn2ncnc2c1 ; CC(C)(C)OC(=O)Nc1ccc(Cc2ccn3ncnc3c2)c(F)c1]

To a mixture of 7-(bromomethyl)-[1,2,4]triazolo[1,5-a]pyridine (1.4 g, 6.60 mmol) and tert-butyl N-[3-fluoro-4-(4,4,5,5-tetramethyl-1,3,2-dioxaborolan-2-yl)phenyl]carbamate (2.6 g, 7.922 mmol) in 1,4-dioxane (15.0 mL)/H₂O (1.5 mL) were added K₂CO₃ (2.7 g, 19.80 mmol) and Pd(dppf)Cl₂ (483.0 mg, 0.66 mmol) at room temperature under N₂. The resulting mixture was stirred at 80° C. for 16 h. After the reaction was completed, the resulting mixture was diluted with water and extracted with ethyl acetate. The combined organic layer was washed with brine, dried over anhydrous Na₂SO₄ and filtered. The filtrate was concentrated under reduced pressure. The residue was purified by flash column chromatography with petroleum ether/ethyl acetate (8/2, v/v) to afford tert-butyl N-(3-fluoro-4-[[1,2,4]triazolo[1,5-a]pyridin-7-ylmethyl]phenyl)carbamate (790.0 mg, 54%) as a yellow solid. LCMS (ESI, m/z): [M+H]⁺=343.0.

### Step 3: Synthesis of 3-fluoro-4-[[1,2,4]triazolo[1,5-a]pyridin-7-ylmethyl]aniline

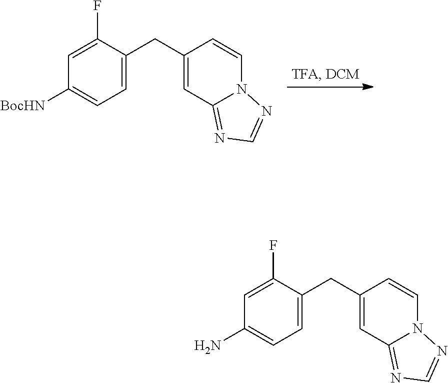 [SMILES: CC(C)(C)OC(=O)Nc1ccc(Cc2ccn3ncnc3c2)c(F)c1 ; Nc1ccc(Cc2ccn3ncnc3c2)c(F)c1]

To a solution of tert-butyl N-(3-fluoro-4-[[1,2,4]triazolo[1,5-a]pyridin-7-ylmethyl]phenyl)carbamate (790.0 mg, 2.30 mmol) in CH₂Cl₂ (4.0 mL) was added TFA (4.0 mL) at room temperature. The resulting mixture was stirred at room temperature for 1 h. After the reaction was completed, the resulting mixture was concentrated under vacuum to afford 3-fluoro-4-[[1,2,4]triazolo[1,5-a]pyridin-7-ylmethyl]aniline (600.0 mg, crude) as a light yellow oil. LCMS (ESI, m/z): [M+H]⁺=243.1.

### Step 4: Synthesis of N-(3-fluoro-4-[[1,2,4]triazolo[1,5-a]pyridin-7-ylmethyl]phenyl)-6-nitroquinazolin-4-amine

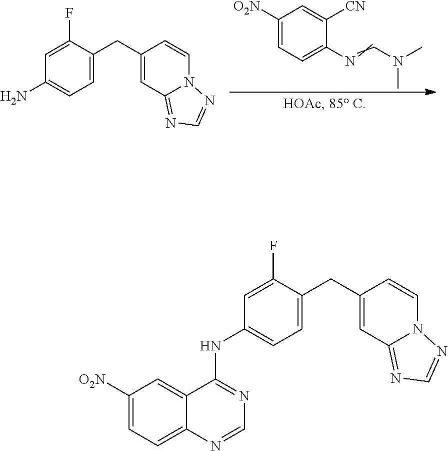 [SMILES: Nc1ccc(Cc2ccn3ncnc3c2)c(F)c1 ; CN(C)C=Nc1ccc([N+](=O)[O-])cc1C#N ; O=[N+]([O-])c1ccc2ncnc(Nc3ccc(Cc4ccn5ncnc5c4)c(F)c3)c2c1]

To a solution of 3-fluoro-4-[[1,2,4]triazolo[1,5-a]pyridin-7-ylmethyl]aniline (500.0 mg, crude) in HOAc (5.0 mL) was added N′-(2-cyano-4-nitrophenyl)-N,N-dimethylformimidamide (450.3 mg, 2.06 mmol) at room temperature. The resulting mixture was stirred at 85° C. for 2 h. After the reaction was completed, the filtrate was concentrated under reduced pressure. The residue was purified by flash column chromatography with CH₂Cl₂/CH₃OH (9/1, v/v) to afford N-(3-fluoro-4-[[1,2,4]triazolo[1,5-a]pyridin-7-ylmethyl]phenyl)-6-nitroquinazolin-4-amine (450.0 mg, 47%) as a yellow solid. LCMS (ESI, m/z): [M+H]⁺=416.1.

### Step 5: Synthesis of N4-(4-([1,2,4]triazolo[1,5-a]pyridin-7-ylmethyl)-3-fluorophenyl)quinazoline-4,6-diamine

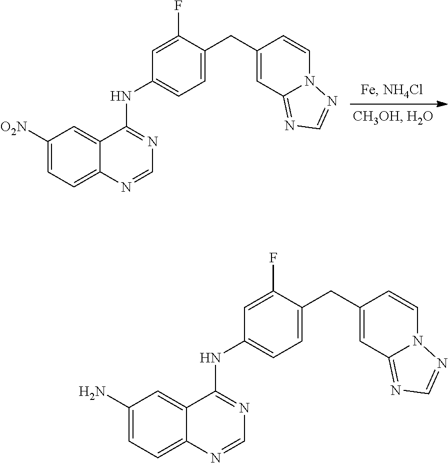 [SMILES: O=[N+]([O-])c1ccc2ncnc(Nc3ccc(Cc4ccn5ncnc5c4)c(F)c3)c2c1 ; Nc1ccc2ncnc(Nc3ccc(Cc4ccn5ncnc5c4)c(F)c3)c2c1]

To a mixture of N-(4-([1,2,4]triazolo[1,5-a]pyridin-7-ylmethyl)-3-fluorophenyl)-6-nitroquinazolin-4-amine (450.0 mg, 1.08 mmol) and NH₄Cl (580.0 mg, 10.8 mmol) in CH₃OH (10.0 mL)/H₂O (2.0 mL) was added Fe (604.8 mg, 10.8 mmol) at room temperature. The resulting mixture was stirred at 80° C. for 1 h. After the reaction was completed, the resulting mixture was concentrated under vacuum. The residue was purified by flash column chromatography with CH₂Cl₂/CH₃OH (9/1, v/v) to afford N4-(4-([1,2,4]triazolo[1,5-a]pyridin-7-ylmethyl)-3-fluorophenyl)quinazoline-4,6-diamine (200.0 mg, 48%) as a yellow solid. LCMS (ESI, m/z): [M+H]⁺=386.1.

### Step 6: Synthesis of N-[4-[(3-fluoro-4-[[1,2,4]triazolo[1,5-a]pyridin-7-ylmethyl]phenyl)amino]quinazolin-6-yl]-5-(oxan-2-yloxy)pent-2-ynamide

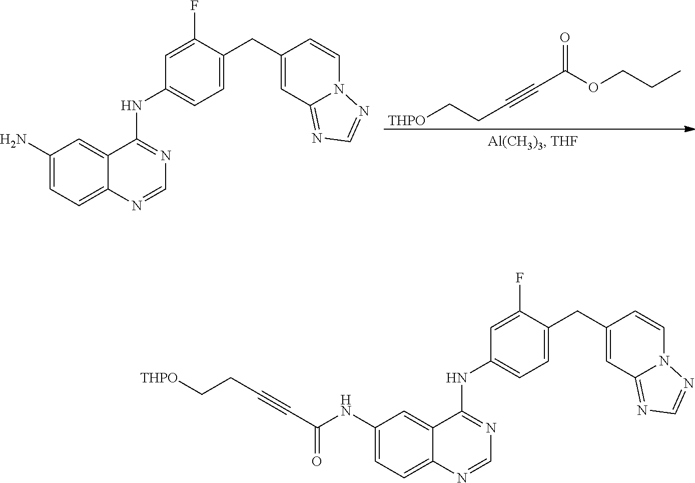

To a mixture of N4-(3-fluoro-4-[[1,2,4]triazolo[1,5-a]pyridin-7-ylmethyl]phenyl)quinazoline-4,6-diamine (120.0 mg, 0.31 mmol) and propyl 5-((tetrahydro-2H-pyran-2-yl)oxy)pent-2-ynoate (211.3 mg, 0.93 mmol) in THF (10.0 mL, 123.43 mmol) was added dropwise trimethylaluminium (0.78 mL, 2 mol/L) at 0° C. The resulting mixture was stirred at 80° C. for 3 h. After the reaction was completed, the reaction mixture was quenched with sat. NH₄Cl (aq.) at room temperature. The resulting mixture was diluted with H₂O and extracted with ethyl acetate. The combined organic layer was washed with brine, dried over anhydrous Na₂SO₄ and filtered. The filtrate was concentrated under reduced pressure. The residue was purified by flash column chromatography with CH₂Cl₂/CH₃OH (9/1, v/v) to afford N-[4-[(3-fluoro-4-[[1,2,4]triazolo[1,5-a]pyridin-7-ylmethyl]phenyl)amino]quinazolin-6-yl]-5-(oxan-2-yloxy)pent-2-ynamide (90.0 mg, 51%) as a yellow solid. LCMS (ESI, m/z): [M+H]⁺=566.2.

### Step 7: Synthesis of N-[4-[(3-fluoro-4-[[1,2,4]triazolo[1,5-a]pyridin-7-ylmethyl]phenyl)amino]quinazolin-6-yl]-5-hydroxypent-2-ynamide (Compound 153)

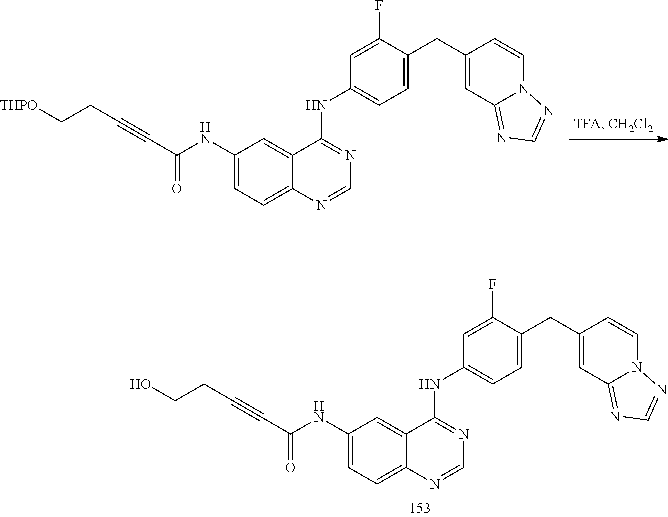

A solution of N-[4-[(3-fluoro-4-[[1,2,4]triazolo[1,5-a]pyridin-7-ylmethyl]phenyl)amino]quinazolin-6-yl]-5-(oxan-2-yloxy)pent-2-ynamide (100.00 mg, 0.17 mmol) and TFA (2.0 mL) in CH₂Cl₂ (2.0 mL) was stirred at room temperature for 1 h. The resulting mixture was concentrated under vacuum. The residue was purified by reverse flash chromatography with H₂O/ACN (1/1, v/v) and then purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 10% B to 22% B in 10 min, 254/220 nm) to afford N-[4-[(3-fluoro-4-[[1,2,4]triazolo[1,5-a]pyridin-7-ylmethyl]phenyl)amino]quinazolin-6-yl]-5-hydroxypent-2-ynamide (Compound 153) (14.3 mg, 16%) as a light yellow solid. LCMS (ESI, m/z): [M+H]⁺=482.2. ¹H NMR (300 MHz, DMSO-d₆): δ 10.95 (s, 1H), 9.93 (s, 1H), 8.86 (d, J=6.9 Hz, 1H), 8.71 (s, 1H), 8.55 (s, 1H), 8.43 (s, 1H), 7.88-7.75 (m, 3H), 7.63-7.57 (m, 2H), 7.41-7.35 (m, 1H), 7.07-7.04 (m, 1H), 5.04 (s, 1H), 4.11 (s, 2H), 3.76-3.62 (m, 2H), 2.79-2.72 (m, 2H).

### Example S44. Synthesis of N-[4-([4-[(6-cyclopropylpyridin-3-yl)oxy]-3-methylphenyl]amino)quinazolin-6-yl]but-2-ynamide (Compound 154)

### Step 1: Synthesis of 5-(benzyloxy)-2-bromopyridine

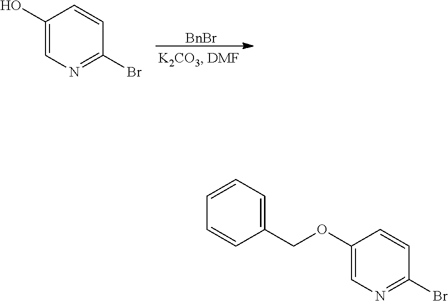 [SMILES: Oc1ccc(Br)nc1 ; "BnBr" ; Brc1ccc(OCc2ccccc2)cn1]

A mixture of 6-bromopyridin-3-ol (5.0 g, 28.74 mmol), benzyl bromide (5.9 g, 34.48 mmol) and K₂CO₃ (11.9 g, 86.21 mmol) in DMF (70.0 mL) was stirred at 50° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (75/25, v/v) to afford 5-(benzyloxy)-2-bromopyridine (5.9 g, 77%) as a white solid. LCMS (ESI, m/z): [M+H]⁺=264.0.

### Step 2: Synthesis of 5-(benzyloxy)-2-cyclopropylpyridine

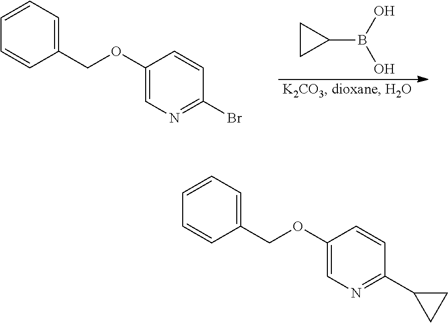

A solution of 5-(benzyloxy)-2-bromopyridine (5.0 g, 18.93 mmol), cyclopropylboronic acid (4.9 g, 56.81 mmol), K₂CO₃ (7.9 g, 56.79 mmol) and Pd(PPh₃)₄ (4.4 g, 3.79 mmol) in dioxane (100.0 mL) and H₂O (20.0 mL) was stirred at 80° C. for 16 h under N2. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford 5-(benzyloxy)-2-cyclopropylpyridine (1.1 g, 25%) as a yellow solid. LCMS (ESI, m/z): [M+H]⁺=226.1.

### Step 3: Synthesis of 6-cyclopropylpyridin-3-ol

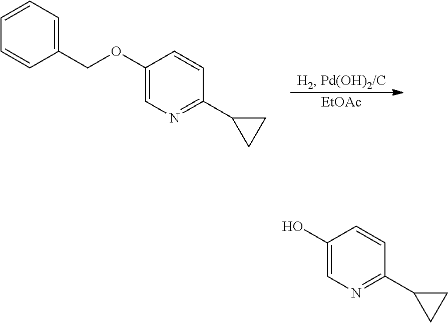 [SMILES: "EtOAc" ; c1ccc(COc2ccc(C3CC3)nc2)cc1 ; "H2, Pd(OH)2/C" ; Oc1ccc(C2CC2)nc1]

To a solution of 5-(benzyloxy)-2-cyclopropylpyridine (1.2 g, 5.33 mmol) in CH₃OH (30.0 mL) was added Pd(OH)₂/C (299.2 mg, 20%) at room temperature. The mixture was stirred at room temperature for 3 h under H₂. After the reaction was completed, the resulting mixture was filtered. The filtrate was concentrated under reduced pressure. The residue was purified by flash column chromatography with CH₂Cl₂/CH₃OH (94/6, v/v) to afford 6-cyclopropylpyridin-3-ol (700.0 mg, 97%) as a yellow solid. LCMS (ESI, m/z): [M+H]⁺=136.1.

### Step 4: Synthesis of 2-cyclopropyl-5-(2-methyl-4-nitrophenoxy)pyridine

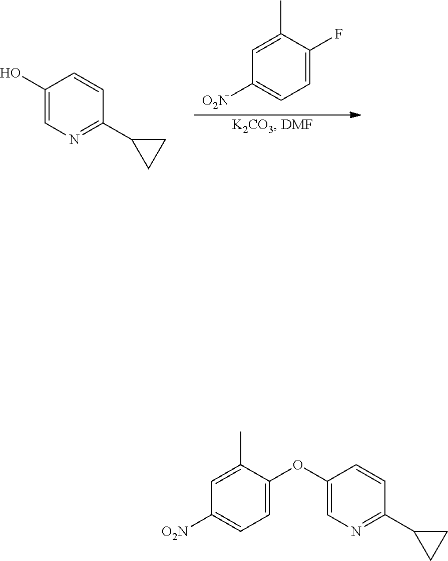

A solution of 6-cyclopropylpyridin-3-ol (650.0 mg, 4.81 mmol), 1-fluoro-2-methyl-4-nitrobenzene (895.2 mg, 5.77 mmol) and K₂CO₃ (1329.2 mg, 9.62 mmol) in DMF (20.0 mL) was stirred at 50° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford 2-cyclopropyl-5-(2-methyl-4-nitrophenoxy)pyridine (508.0 mg, 39%) as a yellow solid. LCMS (ESI, m/z): [M+H]⁺=271.1.

### Step 5: Synthesis of 4-[(6-cyclopropylpyridin-3-yl)oxy]-3-methylaniline

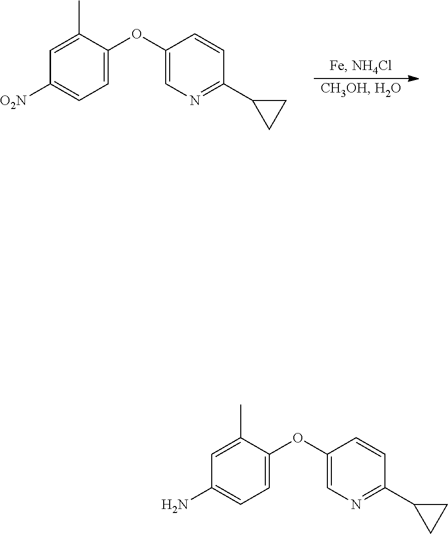

A mixture of 2-cyclopropyl-5-(2-methyl-4-nitrophenoxy)pyridine (450.0 mg, 1.67 mmol), Fe (929.8 mg, 16.65 mmol) and NH₄Cl (890.6 mg, 16.65 mmol) in CH₃OH (20.0 mL) and H₂O (4.0 mL) was stirred at 80° C. for 1 h. After the reaction was completed, the resulting mixture was filtered. The filtrate was concentrated under reduced pressure. The residue was purified by flash column chromatography with CH₂Cl₂/CH₃OH (94/6, v/v) to afford 4-[(6-cyclopropylpyridin-3-yl)oxy]-3-methylaniline (375.0 mg, 93%) as a yellow oil. LCMS (ESI, m/z): [M+H]⁺=241.1.

### Step 6: Synthesis of N-[4-[(6-cyclopropylpyridin-3-yl)oxy]-3-methylphenyl]-6-nitroquinazolin-4-amine

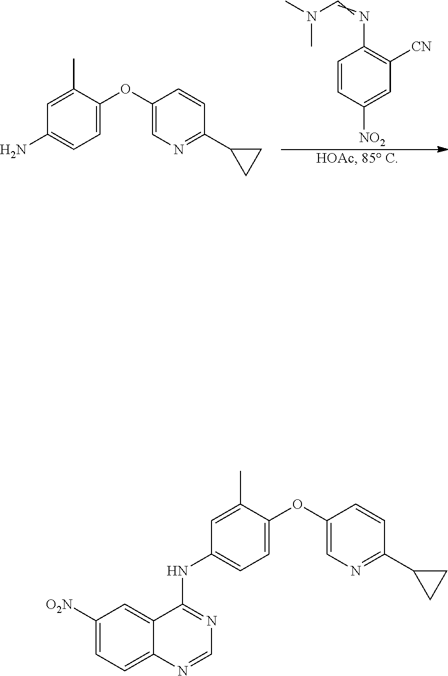

A mixture of 4-[(6-cyclopropylpyridin-3-yl)oxy]-3-methylaniline (300.0 mg, 1.25 mmol) and N′-(2-cyano-4-nitrophenyl)-N,N-dimethylformimidamide (326.9 mg, 1.50 mmol) in HOAc (10.0 mL) was stirred at 85° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford N-[4-[(6-cyclopropylpyridin-3-yl)oxy]-3-methylphenyl]-6-nitroquinazolin-4-amine (420.0 mg, 81%) as a yellow solid. LCMS (ESI, m/z): [M+H]⁺=414.1.

### Step 7: Synthesis of N4-[4-[(6-cyclopropylpyridin-3-yl)oxy]-3-methylphenyl]quinazoline-4,6-diamine

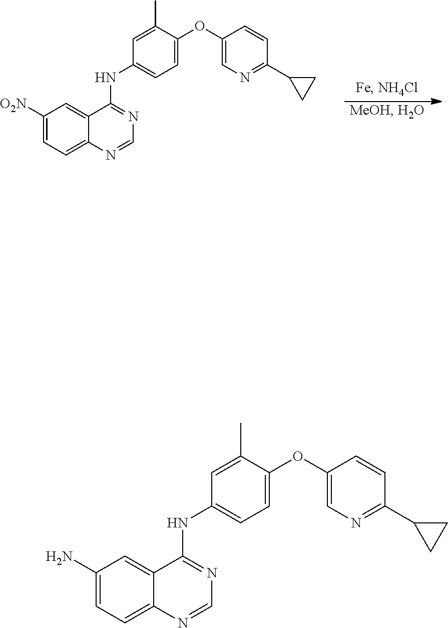

A solution of N-[4-[(6-cyclopropylpyridin-3-yl)oxy]-3-methylphenyl]-6-nitroquinazolin-4-amine (350.0 mg, 0.85 mmol), Fe (472.8 mg, 8.47 mmol) and NH₄Cl (452.8 mg, 8.47 mmol) in CH₃OH (10.0 mL) and H₂O (2.0 mL) was stirred at 80° C. for 1 h. After the reaction was completed, the resulting mixture was filtered. The filtrate was concentrated under reduced pressure. The residue was purified by flash column chromatography with CH₂Cl₂/CH₃OH (92/8, v/v) to afford N4-[4-[(6-cyclopropylpyridin-3-yl)oxy]-3-methylphenyl]quinazoline-4,6-diamine (250.0 mg, 77%) as a yellow solid. LCMS (ESI, m/z): [M+H]⁺=384.2.

### Step 8: Synthesis of N-[4-([4-[(6-cyclopropylpyridin-3-yl)oxy]-3-methylphenyl]amino)quinazolin-6-yl]but-2-ynamide (Compound 154)

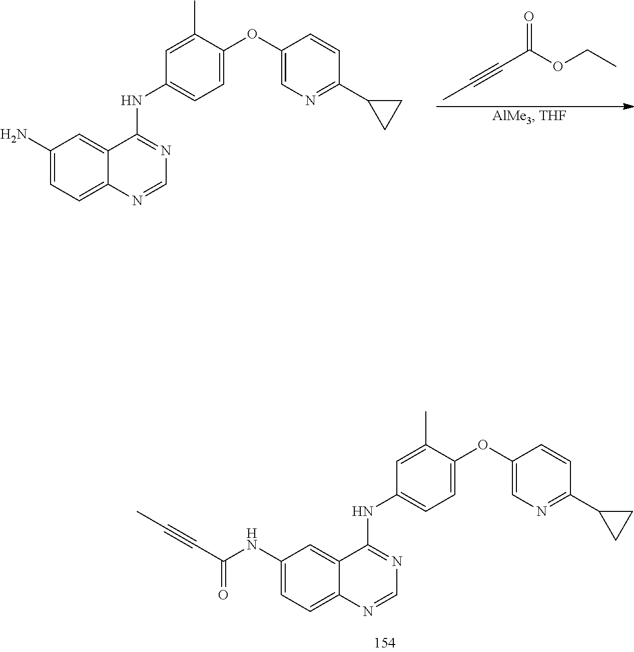

To a mixture of N4-[4-[(6-cyclopropylpyridin-3-yl)oxy]-3-methylphenyl]quinazoline-4,6-diamine (200.0 mg, 0.522 mmol) and but-2-ynoic acid ethyl ester (175.5 mg, 1.57 mmol) in THF (10.0 mL) was added dropwise trimethylaluminium (1.3 mL, 2 mol/L in toluene) at 0° C. The resulting mixture was stirred at 80° C. for 3 h. After the reaction was completed, the reaction mixture was quenched with saturated NH₄Cl solution at room temperature. The resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (90/10, v/v) and then purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 48% B to 58% B in 8 min; 254/220 nm) to afford N-[4-([4-[(6-cyclopropylpyridin-3-yl)oxy]-3-methylphenyl]amino)quinazolin-6-yl]but-2-ynamide (Compound 154) (62.6 mg, 26%) as a white solid. LCMS (ESI, m/z): [M+H]⁺=450.2. ¹H NMR (300 MHz, DMSO-d₆): δ 10.92 (s, 1H), 9.80 (s, 1H), 8.74 (s, 1H), 8.51 (s, 1H), 8.17 (d, J=2.7 Hz, 1H), 7.81-7.73 (m, 3H), 7.65-7.61 (m, 1H), 7.28 (d, J=8.4 Hz, 1H), 7.21-7.17 (m, 1H), 6.94 (d, J=8.7 Hz, 1H), 2.21 (s, 3H), 2.13-2.05 (m, 4H), 0.95-0.86 (m, 4H).

### Example S45. Synthesis of 6-hydroxy-N-[4-[(3-methyl-4-[[1,2,4]triazolo[1,5-a]pyridin-7-yloxy]phenyl)amino]quinazolin-6-yl]hex-2-ynamide (Compound 155)

### Step 1: Synthesis of propyl 6-(oxan-2-yloxy)hex-2-ynoate

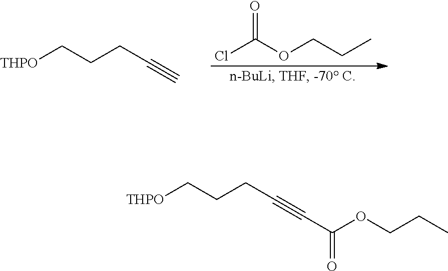

To a solution of 2-(pent-4-yn-1-yloxy)oxane (1.0 g, 5.94 mmol) in THF (20.0 mL) was added n-BuLi (2.8 mL, 2.5 mol/L) at −70° C. under N₂. The mixture was stirred at −70° C. for 1 h. Then propyl carbonochloridate (0.9 g, 7.13 mmol) was added dropwise to the mixture at −70° C. The mixture was stirred at −70° C. for another 2 h. After the reaction was completed, the mixture was quenched with NH₄Cl solution and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (84/16, v/v) to afford propyl 6-(oxan-2-yloxy)hex-2-ynoate (820.0 mg, 54%) as a colorless oil.

### Step 2: Synthesis of N-[4-[(3-methyl-4-[[1,2,4]triazolo[1,5-a]pyridin-7-yloxy]phenyl)amino]quinazolin-6-yl]-6-(oxan-2-yloxy)hex-2-ynamide

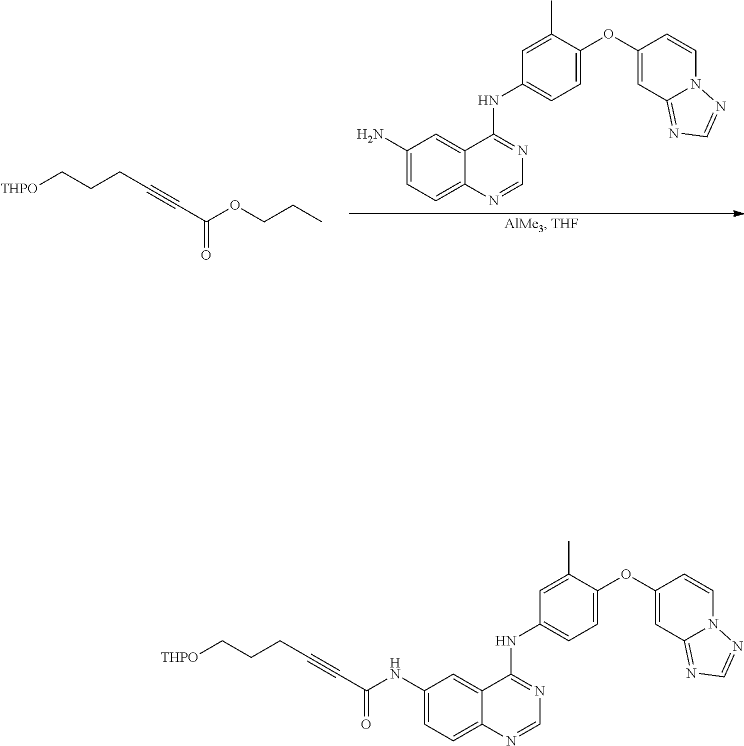

To a mixture of N4-(3-methyl-4-[[1,2,4]triazolo[1,5-a]pyridin-7-yloxy]phenyl)quinazoline-4,6-diamine (300.0 mg, 0.78 mmol) and propyl 6-(oxan-2-yloxy)hex-2-ynoate (597.0 mg, 2.35 mmol) in THF (20.0 mL) was added trimethylaluminium (2.0 mL, 2.0 mol/L in toluene) at 0° C. The resulting mixture was stirred at 80° C. for 3 h. After the reaction was completed, the reaction mixture was quenched with NH₄Cl solution at room temperature. The resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford N-[4-[(3-methyl-4-[[1,2,4]triazolo[1,5-a]pyridin-7-yloxy]phenyl)amino]quinazolin-6-yl]-6-(oxan-2-yloxy)hex-2-ynamide (300.0 mg, 66%) as a yellow oil. LCMS (ESI, m/z): [M+H]⁺=578.2

### Step 3: Synthesis of 6-hydroxy-N-[4-[(3-methyl-4-[[1,2,4]triazolo[1,5-a]pyridin-7-yloxy]phenyl)amino]quinazolin-6-yl]hex-2-ynamide (Compound 155)

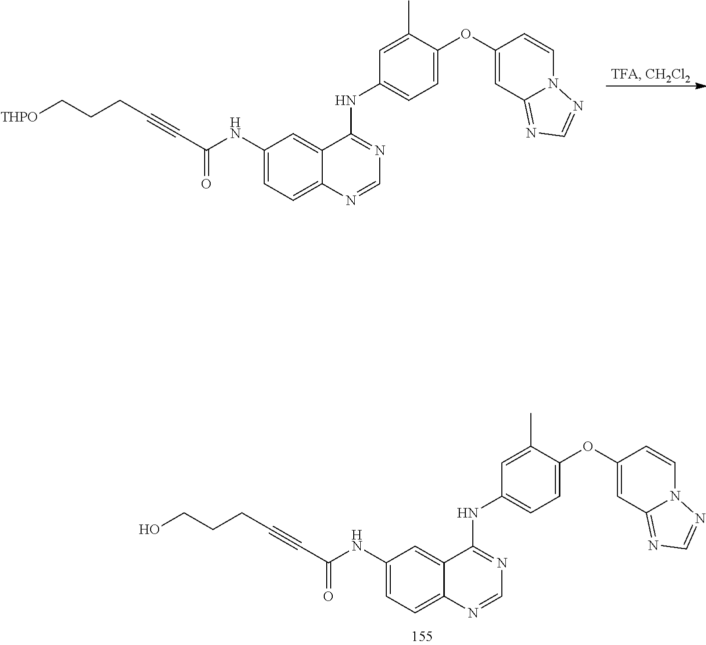

A solution of N-[4-[(3-methyl-4-[[1,2,4]triazolo[1,5-a]pyridin-7-yloxy]phenyl)amino]quinazolin-6-yl]-6-(oxan-2-yloxy)hex-2-ynamide (300.0 mg, 0.52 mmol) and TFA (5.0 mL) in CH₂Cl₂ (5.0 mL) was stirred at room temperature for 2 h. After the reaction was completed, the resulting mixture was diluted with H₂O and extracted with CH₂Cl₂. The combined organic layer was washed with brine, dried over anhydrous sodium sulfate and filtered. The filtrate was concentrated under vacuum. The residue was purified by flash column chromatography with CH₂Cl₂/CH₃OH (90/10, v/v) and then purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 30% B to 40% B in 7 min, 254/220 nm) to afford 6-hydroxy-N-[4-[(3-methyl-4-[[1,2,4]triazolo[1,5-a]pyridin-7-yloxy]phenyl)amino]quinazolin-6-yl]hex-2-ynamide (Compound 155) (61.6 mg, 24%) as a white solid. LCMS (ESI, m/z): [M+H]⁺=494.2. ¹H NMR (400 MHz, CD₃OD): δ 8.75 (d, J=7.6 Hz, 1H), 8.67 (d, J=2.0 Hz, 1H), 8.51 (s, 1H), 8.30 (s, 1H), 7.84-7.71 (m, 4H), 7.19 (d, J=8.8 Hz, 1H), 7.10-7.07 (m, 1H), 6.86 (d, J=2.4 Hz, 1H), 3.74-3.70 (m, 2H), 2.59-2.55 (m, 2H), 2.26 (s, 3H), 1.90-1.83 (m, 2H).

### Example S46. Synthesis of 3-cyclopropyl-N-[4-[(3-methyl-4-[[1,2,4]triazolo[1,5-a]pyridin-7-yloxy]phenyl)amino]quinazolin-6-yl]prop-2-ynamide (Compound 156)

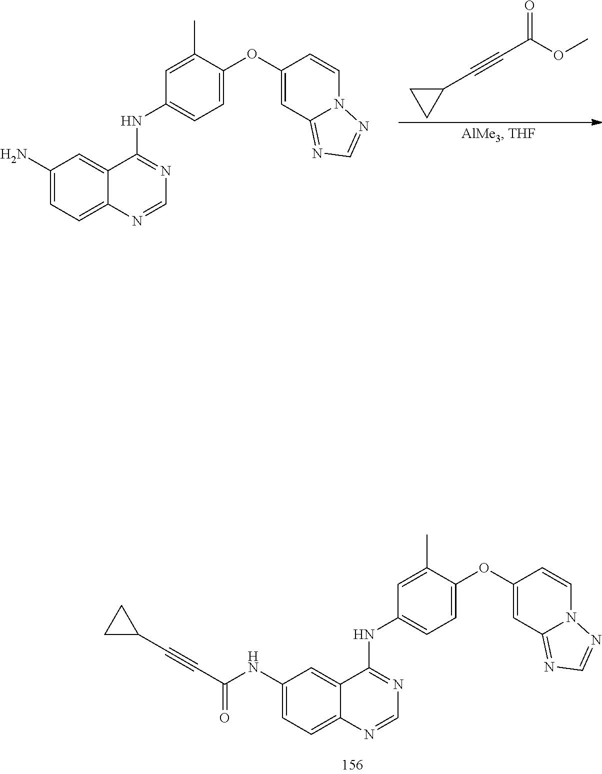

To a mixture of N4-(3-methyl-4-[[1,2,4]triazolo[1,5-a]pyridin-7-yloxy]phenyl)quinazoline-4,6-diamine (200.0 mg, 0.52 mmol) and methyl 3-cyclopropylprop-2-ynoate (194.3 mg, 1.57 mmol) in THF (10.0 mL) was added AlMe₃ (1.3 mL, 2 mol/L) dropwise at 0° C. The resulting mixture was stirred at 80° C. for 3 h. After the reaction was completed, the reaction mixture was quenched with sat. NH₄Cl (aq.) at room temperature. The resulting mixture was extracted with CH₂Cl₂. The combined organic layer was washed with brine, dried over anhydrous Na₂SO₄ and filtered. The filtrate was concentrated under reduced pressure. The residue was purified by Prep-HPLC with the following conditions: Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 39% B to 49% B in 8 min; 254/220 nm; RT1:7.6 min to afford 3-cyclopropyl-N-[4-[(3-methyl-4-[[1,2,4]triazolo[1,5-a]pyridin-7-yloxy]phenyl)amino]quinazolin-6-yl]prop-2-ynamide (Compound 156) (37.1 mg, 14%) as a white solid. LCMS (ESI, m/z): [M+H]⁺=476.3. ¹H NMR (300 MHz, DMSO-d₆): δ 10.86 (s, 1H), 9.88 (s, 1H), 8.96-8.93 (m, 1H), 8.73 (s, 1H), 8.55 (s, 1H), 8.39 (s, 1H), 7.83-7.76 (m, 4H), 7.21 (d, J=8.7 Hz, 1H), 7.06-7.02 (m, 1H), 6.81 (d, J=2.4 Hz, 1H), 2.20 (s, 3H), 1.64-1.59 (m, 1H), 1.02-0.98 (m, 2H), 0.90-0.88 (m, 2H).

### Example S47. Synthesis of 3-cyclopropyl-N-[5-methoxy-4-[(3-methyl-4-[[1,2,4]triazolo[1,5-a]pyridin-7-yloxy]phenyl)amino]quinazolin-6-yl]prop-2-ynamide (Compound 157)

### Step 1: Synthesis of 5-methoxy-6-nitro-3H-quinazolin-4-one

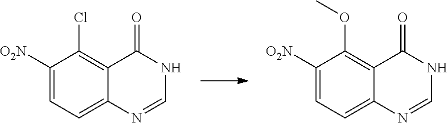 [SMILES: O=c1[nH]cnc2ccc([N+](=O)[O-])c(Cl)c12 ; COc1c([N+](=O)[O-])ccc2nc[nH]c(=O)c12]

A mixture of 5-chloro-6-nitro-3H-quinazolin-4-one (3.0 g, 13.30 mmol) and CH₃ONa (2.9 g, 53.19 mmol) in CH₃OH (50.0 mL) and H₂O (10.0 mL) was stirred at 60° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford 5-methoxy-6-nitro-3H-quinazolin-4-one (550.0 mg, 18%) as a yellow solid. LCMS (ESI, m/z): [M+H]⁺=222.0.

### Step 2: Synthesis of 4-chloro-5-methoxy-6-nitroquinazoline

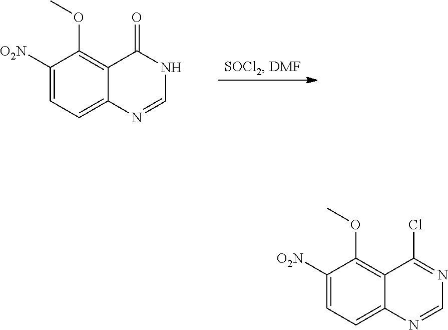

A mixture of 5-methoxy-6-nitro-3H-quinazolin-4-one (420.0 mg, 1.90 mmol), POCl₃ (3.0 mL) and DMF (277.6 mg, 3.80 mmol) in SOCl₂ (3.0 mL) was stirred at 90° C. for 2 h. After the reaction was completed, the resulting mixture was concentrated under vacuum to afford 4-chloro-5-methoxy-6-nitroquinazoline (1.0 g, crude) as a yellow solid. LCMS (ESI, m/z): [M+H]⁺=240.0.

### Step 3: Synthesis of 5-methoxy-N-(3-methyl-4-[[1,2,4]triazolo[1,5-a]pyridin-7-yloxy]phenyl)-6-nitroquinazolin-4-amine

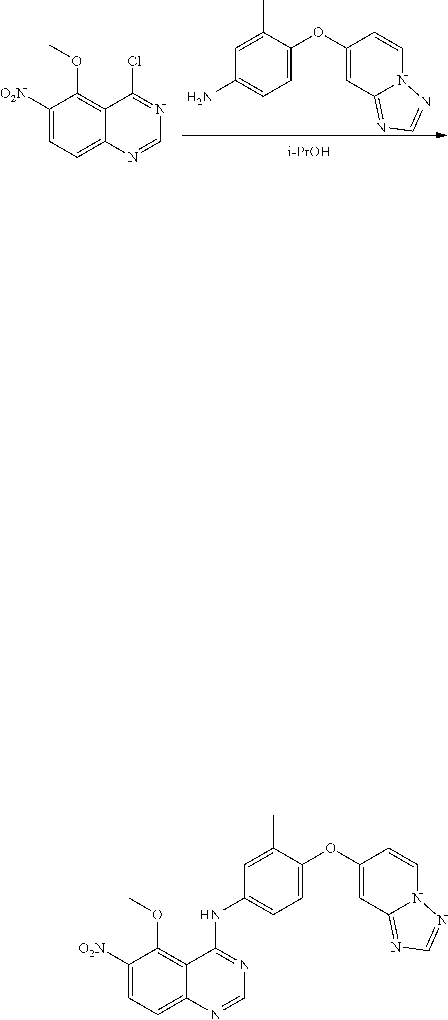 [SMILES: "i-PrOH" ; COc1c([N+](=O)[O-])ccc2ncnc(Cl)c12 ; Cc1cc(N)ccc1Oc1ccn2ncnc2c1 ; COc1c([N+](=O)[O-])ccc2ncnc(Nc3ccc(Oc4ccn5ncnc5c4)c(C)c3)c12]

A solution of 4-chloro-5-methoxy-6-nitroquinazoline (600.0 mg, crude) and 3-methyl-4-[[1,2,4]triazolo[1,5-a]pyridin-7-yloxy]aniline (601.6 mg, 2.50 mmol) in i-PrOH (10.0 mL) was stirred at room temperature for 16 h. After the reaction was completed, the resulting mixture was concentrated under vacuum. The residue was purified by flash column chromatography with CH₂Cl₂/CH₃OH (94/6, v/v) to afford 5-methoxy-N-(3-methyl-4-[[1,2,4]triazolo[1,5-a]pyridin-7-yloxy]phenyl)-6-nitroquinazolin-4-amine (170 mg, 15%) as a yellow solid. LCMS (ESI, m/z): [M+H]⁺=444.1.

### Step 4: Synthesis of 5-methoxy-N4-(3-methyl-4-[[1,2,4]triazolo[1,5-a]pyridin-7-yloxy]phenyl)quinazoline-4,6-diamine

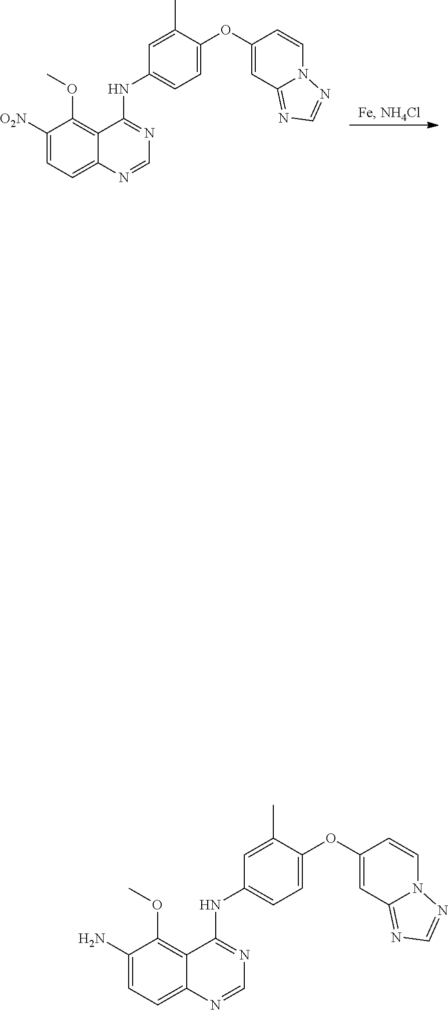

A mixture of 5-methoxy-N-(3-methyl-4-[[1,2,4]triazolo[1,5-a]pyridin-7-yloxy]phenyl)-6-nitroquinazolin-4-amine (380.0 mg, 0.86 mmol), Fe (478.6 mg, 8.57 mmol) and NH₄Cl (458.4 mg, 8.57 mmol) in CH₃OH (10.0 mL) and H₂O (2.0 mL) was stirred at 80° C. for 1 h. After the reaction was completed, the resulting mixture was filtered. The filtrate was concentrated under reduced pressure. The residue was purified by flash column chromatography with CH₂Cl₂/CH₃OH (94/6, v/v) to afford 5-methoxy-N4-(3-methyl-4-[[1,2,4]triazolo[1,5-a]pyridin-7-yloxy]phenyl)quinazoline-4,6-diamine (160.0 mg, 45%) as a yellow oil. LCMS (ESI, m/z): [M+H]⁺=414.2.

### Step 5: Synthesis of 3-cyclopropyl-N-[5-methoxy-4-[(3-methyl-4-[[1,2,4]triazolo[1,5-a]pyridin-7-yloxy]phenyl)amino]quinazolin-6-yl]prop-2-ynamide (Compound 157)

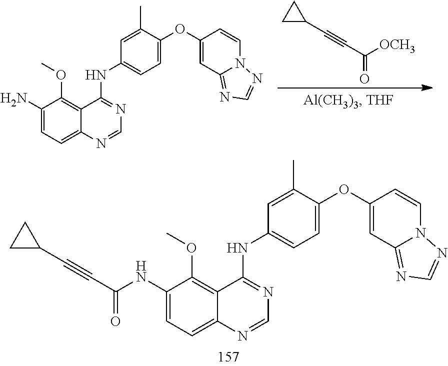 [SMILES: COc1c(N)ccc2ncnc(Nc3ccc(Oc4ccn5ncnc5c4)c(C)c3)c12 ; COC(=O)C#CC1CC1 ; COc1c(NC(=O)C#CC2CC2)ccc2ncnc(Nc3ccc(Oc4ccn5ncnc5c4)c(C)c3)c12]

To a solution of 5-methoxy-N4-(3-methyl-4-[[1,2,4]triazolo[1,5-a]pyridin-7-yloxy]phenyl)quinazoline-4,6-diamine (110.0 mg, 0.27 mmol) and methyl 3-cyclopropylprop-2-ynoate (99.1 mg, 0.80 mmol) in THF (10.0 mL) was added dropwise trimethylaluminium (0.7 mL, 2 mol/L) at 0° C. The resulting mixture was stirred at 80° C. for 3 h. After the reaction was completed, the reaction mixture was quenched with saturated NH₄Cl solution and extracted with CH₂Cl₂. The combined organic layer was washed with brine, dried over anhydrous Na₂SO₄ and filtered. The filtrate was concentrated under vacuum. The residue was purified by flash column chromatography with CH₂Cl₂/CH₃OH (91/9, v/v) and then purified by Prep-HPLC with the following conditions (Column: XBridge Prep C₁₈ OBD Column 30×100 mm, 5 um; Mobile Phase A: Water (10 mmol/L NH₄HCO₃), Mobile Phase B: ACN; Flow rate: 60 mL/min; Gradient: 43% B to 69% B in 9 min; 254/220 nm) to afford 3-cyclopropyl-N-[5-methoxy-4-[(3-methyl-4-[[1,2,4]triazolo[1,5-a]pyridin-7-yloxy]phenyl)amino]quinazolin-6-yl]prop-2-ynamide (Compound 157) (8.1 mg, 5%) as a white solid. LCMS (ESI, m/z): [M+H]⁺=506.1. ¹H NMR (300 MHz, DMSO-d₆+DCl): δ 10.21 (s, 1H), 10.62 (s, 1H), 9.24-9.14 (m, 2H), 8.76 (s, 1H), 7.94 (d, J=9.0 Hz, 1H), 7.71-7.65 (m, 2H), 7.43-7.26 (m, 3H), 7.03 (s, 1H), 4.17 (s, 3H), 2.20 (s, 3H), 1.60-1.55 (m, 1H), 1.00-0.84 (m, 4H).

### Example S48. Synthesis of 3-cyclopropyl-N-[4-[(4-[[1-(cyclopropylmethyl)-2-oxopyridin-4-yl]oxy]-3-methylphenyl)amino]quinazolin-6-yl]prop-2-ynamide (Compound 158)

### Step 1: Synthesis of 4-(benzyloxy)-1-(cyclopropylmethyl)pyridin-2-one

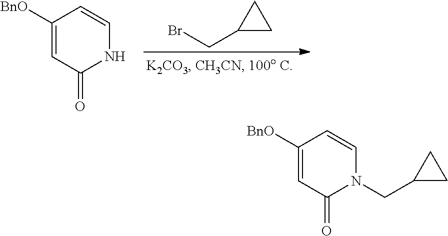 [SMILES: O=c1cc(OCc2ccccc2)cc[nH]1 ; BrCC1CC1 ; O=c1cc(OCc2ccccc2)ccn1CC1CC1]

To a solution of 4-(benzyloxy)-1H-pyridin-2-one (10.0 g, 49.70 mmol) in CH₃CN (400.0 mL) was added (bromomethyl)cyclopropane (7.4 g, 54.67 mmol) and K₂CO₃ (20.6 g, 149.01 mmol) at room temperature under N₂. The resulting mixture was stirred at 100° C. for 16 h under N₂. After the reaction was completed, the mixture was cooled to room temperature and filtered. The filtrate was concentrated under vacuum. The residue was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/EtOAc (1/1, v/v) to afford 4-(benzyloxy)-1-(cyclopropylmethyl)pyridin-2-one (8.5 g, 66%) as a white solid. LCMS (ESI, m/z): [M+H]⁺=256.1.

### Step 2: Synthesis of 1-(cyclopropylmethyl)-4-hydroxypyridin-2-one

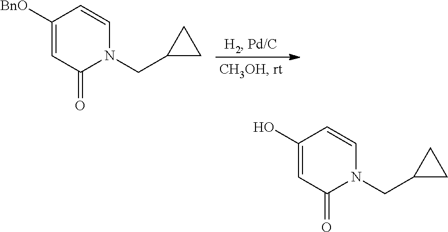 [SMILES: O=c1cc(OCc2ccccc2)ccn1CC1CC1 ; O=c1cc(O)ccn1CC1CC1]

To a solution of 4-(benzyloxy)-1-(cyclopropylmethyl)pyridin-2-one (8.5 g, 33.29 mmol) in MeOH (100.0 mL) was added Pd/C (2.6 g, dry). The mixture was stirred at room temperature for 4 h under H₂. After the reaction was completed, the reaction mixture was filtered. The filtrate was evaporated in vacuo to afford 1-(cyclopropylmethyl)-4-hydroxypyridin-2-one (4.9 g, crude) as a white solid. LCMS (ESI, m/z): [M+H]⁺=166.1.

### Step 3: Synthesis of 1-(cyclopropylmethyl)-4-(2-methyl-4-nitrophenoxy)pyridin-2-one

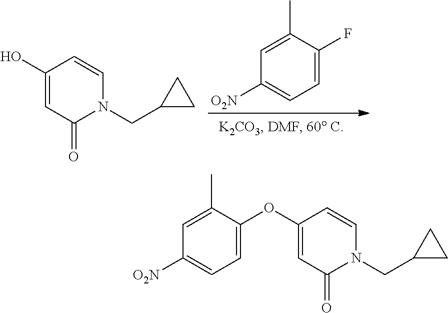

To a solution of 1-(cyclopropylmethyl)-4-hydroxypyridin-2-one (4.9 g, 29.66 mmol) in DMF (49.0 mL) was added 1-fluoro-2-methyl-4-nitrobenzene (4.6 g, 29.66 mmol) and K₂CO₃ (8.2 g, 59.32 mmol) at room temperature. The resulting mixture was stirred at 60° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/EtOAc (1/100, v/v) to afford 1-(cyclopropylmethyl)-4-(2-methyl-4-nitrophenoxy)pyridin-2-one (6.7 g, 75%) as a light yellow solid. LCMS (ESI, m/z): [M+H]⁺=301.1.

### Step 4: Synthesis of 4-(4-amino-2-methylphenoxy)-1-(cyclopropylmethyl)pyridin-2-one

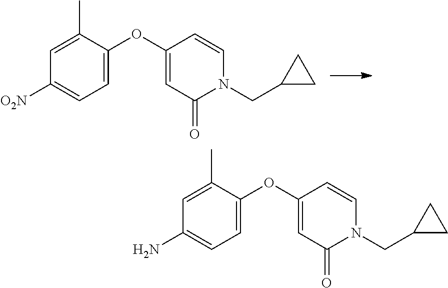

To a solution of 1-(cyclopropylmethyl)-4-(2-methyl-4-nitrophenoxy)pyridin-2-one (500.0 mg, 1.67 mmol) in MeOH (20.0 mL) was added Pd/C (150.0 mg, dry). The mixture was stirred at room temperature for 4 h under H₂. After the reaction was completed, the reaction mixture was filtered. The filtrate was evaporated in vacuo to afford 4-(4-amino-2-methylphenoxy)-1-(cyclopropylmethyl)pyridin-2-one (436.0 mg, crude) as a light yellow solid. LCMS (ESI, m/z): [M+H]⁺=271.1.

### Step 5: Synthesis of 1-(cyclopropylmethyl)-4-[2-methyl-4-[(6-nitroquinazolin-4-yl)amino]phenoxy]pyridin-2-one

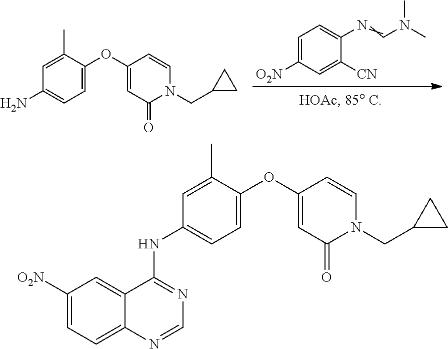 [SMILES: Cc1cc(N)ccc1Oc1ccn(CC2CC2)c(=O)c1 ; CN(C)C=Nc1ccc([N+](=O)[O-])cc1C#N ; Cc1cc(Nc2ncnc3ccc([N+](=O)[O-])cc23)ccc1Oc1ccn(CC2CC2)c(=O)c1]

To a mixture of 4-(4-amino-2-methylphenoxy)-1-(cyclopropylmethyl)pyridin-2-one (400.0 mg, 1.48 mmol) in HOAc (40.0 mL) was added N′-(2-cyano-4-nitrophenyl)-N,N-dimethylformimidamide (322.9 mg, 1.48 mmol) at room temperature. The resulting mixture was stirred at 85° C. for 2 h. After the reaction was completed, the mixture was concentrated under vacuum. The residue was purified by flash column chromatography with CH₂Cl₂/MeOH (10/1, v/v) to afford 1-(cyclopropylmethyl)-4-[2-methyl-4-[(6-nitroquinazolin-4-yl)amino]phenoxy]pyridin-2-one (590.0 mg, 90%) as a yellow solid. LCMS (ESI, m/z): [M+H]⁺=444.2.

### Step 6: Synthesis of 4-[4-[(6-aminoquinazolin-4-yl)amino]-2-methylphenoxy]-1-(cyclopropylmethyl)pyridin-2-one

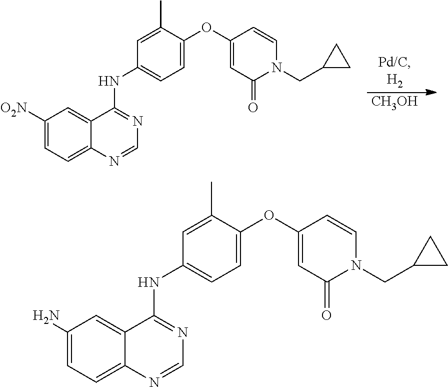

To a solution of 1-(cyclopropylmethyl)-4-[2-methyl-4-[(6-nitroquinazolin-4-yl)amino]phenoxy]pyridin-2-one (590.0 mg, 1.33 mmol) in MeOH (20.0 mL) was added Pd/C (180.0 mg, dry). The mixture was stirred at room temperature for 4 h under H₂. After the reaction was completed, the reaction mixture was filtered. The filtrate was evaporated in vacuo to afford 4-[4-[(6-aminoquinazolin-4-yl)amino]-2-methylphenoxy]-1-(cyclopropylmethyl)pyridin-2-one (390.0 mg, dry) as a yellow solid. LCMS (ESI, m/z): [M+H]⁺=414.2.

### Step 7: Synthesis of 3-cyclopropyl-N-[4-[(4-[[1-(cyclopropylmethyl)-2-oxopyridin-4-yl]oxy]-3-methylphenyl)amino]quinazolin-6-yl]prop-2-ynamide (Compound 158)

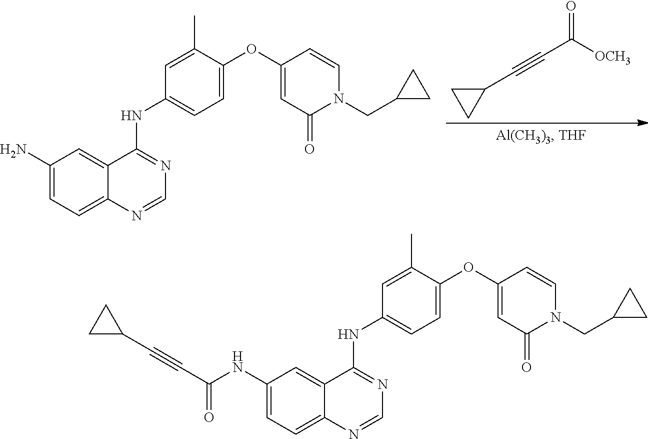

To a mixture of 4-[4-[(6-aminoquinazolin-4-yl)amino]-2-methylphenoxy]-1-(cyclopropylmethyl)pyridin-2-one (170.0 mg, 0.41 mmol) and methyl 3-cyclopropylprop-2-ynoate (255.2 mg, 2.06 mmol) in THF (10.0 mL) was added dropwise AlMe₃ (1.44 mL, 2 mol/L) at 0° C. The resulting mixture was stirred at 80° C. for 3 h under N₂. After the reaction was completed, the reaction mixture was quenched with sat. NH₄Cl (aq.) at room temperature and extracted with CH₂Cl₂. The combined organic layer was washed with brine, dried over anhydrous Na₂SO₄ and filtered. The filtrate was concentrated under reduced pressure. The residue was purified by reverse phase flash with the following conditions: Column: XBridge Prep OBD C18 Column, 30×150 mm, 5 um; Mobile Phase A: Water (10 mmol/1 NH₄HCO₃), Mobile Phase B: ACN; Flow rate: 60 mL/min; Gradient: 37% B to 67% B in 7 min; 254 nm; RT1: 6.5 min to afford 3-cyclopropyl-N-[4-[(4-[[1-(cyclopropylmethyl)-2-oxopyridin-4-yl]oxy]-3-methylphenyl)amino]quinazolin-6-yl]prop-2-ynamide (Compound 158) (54.0 mg, 25%) as a white solid. LCMS (ESI, m/z): [M+H]⁺=506.1. ¹H NMR (300 MHz, DMSO-d₆): δ 10.86 (s, 1H), 9.87 (s, 1H), 8.71 (s, 1H), 8.55 (s, 1H), 7.83-7.72 (m, 5H), 7.13 (d, J=8.7 Hz, 1H), 6.14-6.10 (m, 1H), 5.34 (d, J=2.7 Hz, 1H), 3.68 (d, J=6.9 Hz, 2H), 2.16 (s, 3H), 1.63-1.58 (m, 1H), 1.20-1.15 (m, 1H), 1.02-0.98 (m, 2H), 0.90-0.85 (m, 2H), 0.50-0.44 (m, 2H), 0.38-0.33 (m, 2H).

### Example S49. Synthesis of 3-cyclopropyl-N-{4-[(3-methyl-4-{[2,4]triazolo[1,5-a]pyridin-7-ylsulfanyl}phenyl)amino]quinazolin-6-yl}prop-2-ynamide (Compound 159)

### Step 1: Synthesis of 7-[[(4-methoxyphenyl)methyl]sulfanyl]-[1,2,4]triazolo[1,5-a]pyridine

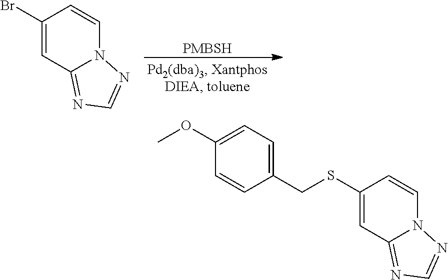

To a mixture of 7-bromo-[1,2,4]triazolo[1,5-a]pyridine (5.0 g, 25.25 mmol) in toluene (25.0 mL) was added (4-methoxyphenyl)methanethiol (3.9 g, 25.25 mmol), DIEA (6.5 g, 50.50 mmol), XantPhos (1.5 g, 2.53 mmol) and Pd₂(dba)₃ (1.1 g, 1.26 mmol) at room temperature. The resulting mixture was stirred at 100° C. for 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/EtOAc (1/10, v/v) to afford 7-[[(4-methoxyphenyl)methyl]sulfanyl]-[1,2,4]triazolo[1,5-a]pyridine (6.0 g, 87%) as an orange solid. LCMS (ESI, m/z): [M+H]⁺=272.1.

### Step 2: Synthesis of [1,2,4]triazolo[1,5-a]pyridine-7-thiol

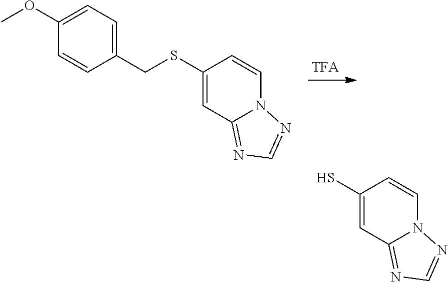 [SMILES: COc1ccc(CSc2ccn3ncnc3c2)cc1 ; Sc1ccn2ncnc2c1]

A solution of 7-[[(4-methoxyphenyl)methyl]sulfanyl]-[1,2,4]triazolo[1,5-a]pyridine (1.5 g, 5.53 mmol) in TFA (60.0 mL) was stirred at 120° C. for 16 h. After the reaction was completed, the resulting mixture was concentrated under reduced pressure to afford [1,2,4]triazolo[1,5-a]pyridine-7-thiol (2.6 g, crude) as a tan oil. LCMS (ESI, m/z): [M+H]⁺=152.2.

### Step 3: Synthesis of 7-[(2-methyl-4-nitrophenyl)sulfanyl]-[1,2,4]triazolo[1,5-a]pyridine

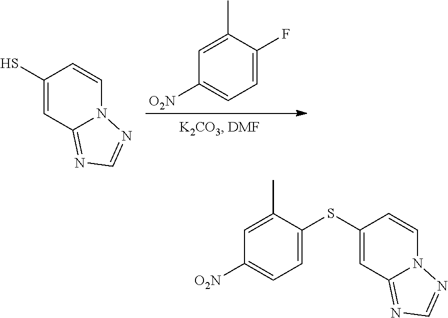 [SMILES: Sc1ccn2ncnc2c1 ; Cc1cc([N+](=O)[O-])ccc1F ; Cc1cc([N+](=O)[O-])ccc1Sc1ccn2ncnc2c1]

To a mixture of [1,2,4]triazolo[1,5-a]pyridine-7-thiol (1.8 g, crude) in DMF (20.0 mL) was added 1-fluoro-2-methyl-4-nitrobenzene (1.8 g, 11.91 mmol) and K₂CO₃ (3.3 g, 23.81 mmol) at room temperature. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 7-[(2-methyl-4-nitrophenyl)sulfanyl]-[1,2,4]triazolo[1,5-a]pyridine (800.0 mg, 23%) as a white solid. LCMS (ESI, m/z): [M+H]⁺=287.1.

### Step 4: Synthesis of 3-methyl-4-[[1,2,4]triazolo[1,5-a]pyridin-7-ylsulfanyl]aniline

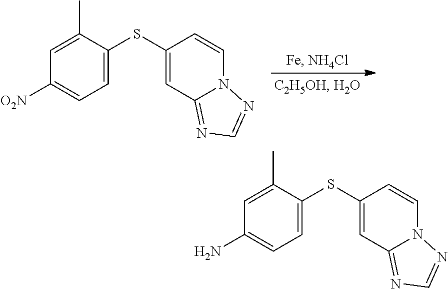

To a solution of 7-[(2-methyl-4-nitrophenyl)sulfanyl]-[1,2,4]triazolo[1,5-a]pyridine (800.0 mg, 2.79 mmol) in EtOH (16.0 mL)/H₂O (3.2 mL) was added NH₄Cl (747.3 mg, 13.97 mmol). Then Fe (780.2 mg, 13.97 mmol) was added to the mixture at 80° C. The resulting mixture was stirred at 80° C. for 3 h. After the reaction was completed, the resulting mixture was filtered. The filtrate was concentrated under reduced pressure. The residue was purified by flash column chromatography with CH₂Cl₂/MeOH (10/1, v/v) to afford 3-methyl-4-[[1,2,4]triazolo[1,5-a]pyridin-7-ylsulfanyl]aniline (500.0 mg, 69%) as a brown oil. LCMS (ESI, m/z): [M+H]⁺=257.1.

### Step 5: Synthesis of N-(3-methyl-4-[[1,2,4]triazolo[1,5-a]pyridin-7-ylsulfanyl]phenyl)-6-nitroquinazolin-4-amine

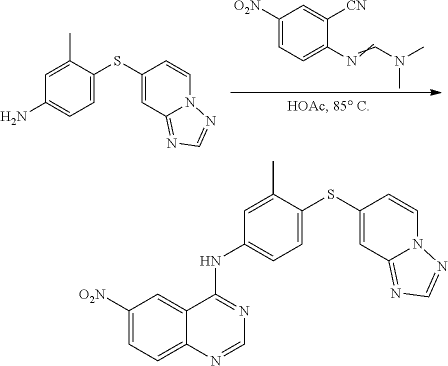 [SMILES: Cc1cc(N)ccc1Sc1ccn2ncnc2c1 ; CN(C)C=Nc1ccc([N+](=O)[O-])cc1C#N ; Cc1cc(Nc2ncnc3ccc([N+](=O)[O-])cc23)ccc1Sc1ccn2ncnc2c1]

To a solution of 3-methyl-4-[[1,2,4]triazolo[1,5-a]pyridin-7-ylsulfanyl]aniline (300.0 mg, 1.17 mmol) in HOAc (5.0 mL) was added N′-(2-cyano-4-nitrophenyl)-N,N-dimethylformimidamide (255.4 mg, 1.17 mmol) at room temperature. The resulting mixture was stirred at 85° C. for 2 h. After the reaction was completed, the resulting mixture was concentrated under vacuo. The residue was purified by flash column chromatography with CH₂Cl₂/MeOH (10/1, v/v) to afford N-(3-methyl-4-[[1,2,4]triazolo[1,5-a]pyridin-7-ylsulfanyl]phenyl)-6-nitroquinazolin-4-amine (425.0 mg, 84%) as a yellow solid. LCMS (ESI, m/z): [M+H]⁺=430.1.

### Step 6: Synthesis of N4-(3-methyl-4-[[1,2,4]triazolo[1,5-a]pyridin-7-ylsulfanyl]phenyl)quinazoline-4,6-diamine

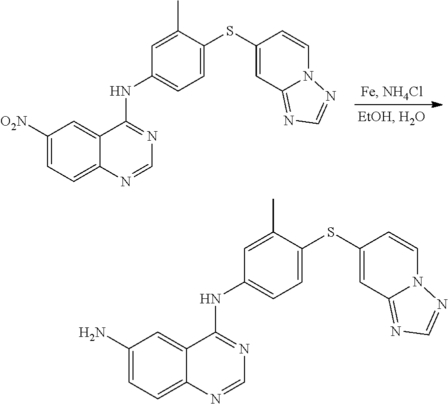 [SMILES: Cc1cc(Nc2ncnc3ccc([N+](=O)[O-])cc23)ccc1Sc1ccn2ncnc2c1 ; Cc1cc(Nc2ncnc3ccc(N)cc23)ccc1Sc1ccn2ncnc2c1]

To a solution of N-(3-methyl-4-[[1,2,4]triazolo[1,5-a]pyridin-7-ylsulfanyl]phenyl)-6-nitroquinazolin-4-amine (362.0 mg, 0.84 mmol) in EtOH (14.0 mL)/H₂O (2.8 mL) was added NH₄Cl (225.4 mg, 4.22 mmol). Then Fe (235.4 mg, 4.22 mmol) was added to the mixture at 80° C. The resulting mixture was stirred at 80° C. for 3 h. After the reaction was completed, the resulting mixture was filtered. The filtrate was concentrated under reduced pressure. The residue was purified by flash column chromatography with CH₂Cl₂/MeOH (10/1, v/v) to afford N4-(3-methyl-4-[[1,2,4]triazolo[1,5-a]pyridin-7-ylsulfanyl]phenyl)quinazoline-4,6-diamine (230.0 mg, 68%) as a yellow solid. LCMS (ESI, m/z): [M+H]⁺=400.1.

### Step 7: Synthesis of 3-cyclopropyl-N-{4-[(3-methyl-4-{[1,2,4]triazolo[1,5-a]pyridin-7-ylsulfanyl}phenyl)amino]quinazolin-6-yl}prop-2-ynamide (Compound 159)

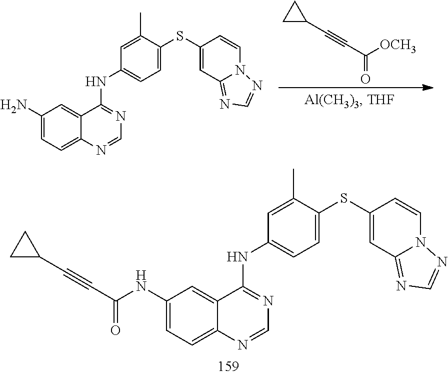 [SMILES: Cc1cc(Nc2ncnc3ccc(N)cc23)ccc1Sc1ccn2ncnc2c1 ; COC(=O)C#CC1CC1 ; Cc1cc(Nc2ncnc3ccc(NC(=O)C#CC4CC4)cc23)ccc1Sc1ccn2ncnc2c1]

To a mixture of N4-(3-methyl-4-[[1,2,4]triazolo[1,5-a]pyridin-7-yloxy]phenyl)quinazoline-4,6-diamine (200.0 mg, 0.52 mmol) and methyl 3-cyclopropylprop-2-ynoate (186.5 mg, 1.50 mmol) in THF (10.0 mL) was added AlMe₃ (1.3 mL, 2 mol/L) at 0° C. The resulting mixture was stirred at 80° C. for 3 h. After the reaction was completed, the reaction was quenched with NH₄Cl (aq) at 0° C. The resulting mixture was diluted with H₂O and extracted with DCM. The combined organic layer was washed with brine, dried over anhydrous sodium sulfate and filtered. The filtrate was concentrated under reduced pressure. The residue was purified by Prep-HPLC with the following conditions: (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 29% B to 59% B in 9 min; 254 nm) to afford 3-cyclopropyl-N-{4-[(3-methyl-4-{[1,2,4]triazolo[1,5-a]pyridin-7-ylsulfanyl}phenyl)amino]quinazolin-6-yl}prop-2-ynamide (Compound 159) (85.5 mg, 34%) as a white solid. LCMS (ESI, m/z): [M+H]⁺=492.1. ¹H NMR (300 MHz, DMSO-d₆): δ 10.86 (s, 1H), 9.98 (s, 1H), 8.84-8.81 (m, 1H), 8.73 (s, 1H), 8.60 (s, 1H), 8.39 (s, 1H), 7.97-7.89 (m, 2H), 7.80-7.79 (m, 2H), 7.62 (d, J=8.4 Hz, 1H), 7.06 (d, J=1.5 Hz, 1H), 6.93-6.90 (m, 1H), 2.37 (s, 3H), 1.65-1.55 (m, 1H), 1.00-0.97 (m, 2H), 0.87-0.84 (m, 2H).

### Example S50. Synthesis of 3-cyclopropyl-N-[4-([4-[(1-ethyl-2-oxopyridin-4-yl)sulfanyl]-3-methylpheny]amino)quinazolin-6-yl]prop-2-ynamide (Compound 160)

### Step 1: Synthesis of 1-ethyl-4-sulfanylpyridin-2-one

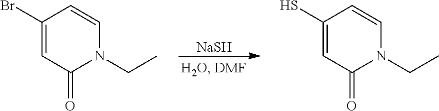

To a solution of 4-bromo-1-ethylpyridin-2-one (1.3 g, 6.68 mmol) in DMF/H₂O (10.0 mL/5.0 mL) was added NaSH (0.7 g, 13.36 mmol) at room temperature. The resulting mixture was stirred at 60° C. for 1 h. After the reaction was completed, the reaction mixture was diluted with H₂O and extracted with ethyl acetate, the combined organic layer was washed with brine, dried over anhydrous sodium sulfate and filtered. The filtrate was concentrated under vacuum. The residue was purified by flash column chromatography with petroleum ether/ethyl acetate (80/20, v/v) to afford 1-ethyl-4-sulfanylpyridin-2-one (800.0 mg, 77%) as a yellow solid. LCMS (ESI, m/z): [M+H]⁺=156.0.

### Step 2: Synthesis of 1-ethyl-4-[(2-methyl-4-nitrophenyl)sulfanyl]pyridin-2-one

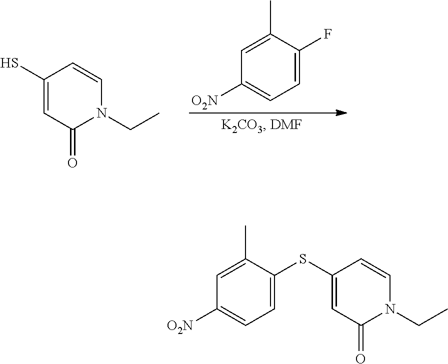

To a solution of 1-ethyl-4-sulfanylpyridin-2-one (750.0 mg, 4.83 mmol) in DMF (10.0 mL) was added 1-fluoro-2-methyl-4-nitrobenzene (749.6 mg, 4.83 mmol) and K₂CO₃ (2.0 g, 14.50 mmol) at room temperature. The mixture was stirred at 120° C. for 2 h. After the reaction was completed, the mixture was diluted with water and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (48/52, v/v) to afford 1-ethyl-4-[(2-methyl-4-nitrophenyl)sulfanyl]pyridin-2-one (1.0 g, 71%) as a yellow solid. LCMS (ESI, m/z): [M+H]⁺=291.1.

### Step 3: Synthesis of 4-[(4-amino-2-methylphenyl)sulfanyl]-1-ethylpyridin-2-one

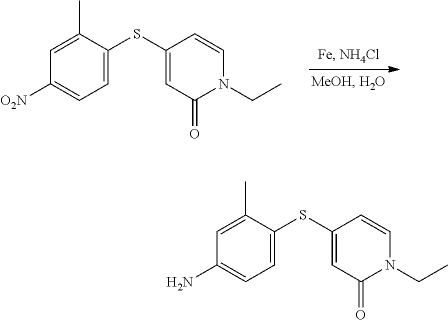 [SMILES: CCn1ccc(Sc2ccc([N+](=O)[O-])cc2C)cc1=O ; CCn1ccc(Sc2ccc(N)cc2C)cc1=O]

To a solution of 1-ethyl-4-[(2-methyl-4-nitrophenyl)sulfanyl]pyridin-2-one (950.0 mg, 3.27 mmol) in MeOH/H₂O (10.0 mL/2.0 mL) was added Fe (1.3 g, 22.90 mmol) and NH₄Cl (1.2 g, 22.90 mmol). The mixture was stirred at 70° C. for 3 h. After the reaction was completed, the resulting mixture was filtered. The filtrate was concentrated under vacuum. The residue was purified by flash column chromatography with CH₂Cl₂/MeOH (92/8, v/v) to afford 4-[(4-amino-2-methylphenyl)sulfanyl]-1-ethylpyridin-2-one (800.0 mg, 93%) as a yellow solid. LCMS (ESI, m/z): [M+H]⁺=261.1.

### Step 4: Synthesis of 1-ethyl-4-([2-methyl-4-[(6-nitroquinazolin-4-yl)amino]phenyl]sulfanyl)pyridin-2-one

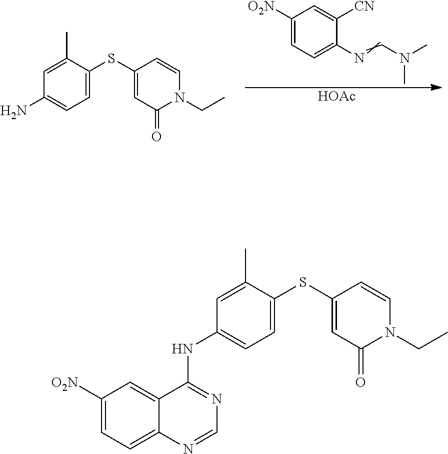 [SMILES: CCn1ccc(Sc2ccc(N)cc2C)cc1=O ; CN(C)C=Nc1ccc([N+](=O)[O-])cc1C#N ; CCn1ccc(Sc2ccc(Nc3ncnc4ccc([N+](=O)[O-])cc34)cc2C)cc1=O ; "HOAc"]

To a solution of 4-[(4-amino-2-methylphenyl)sulfanyl]-1-ethylpyridin-2-one (800.0 mg, 3.07 mmol) in AcOH (15.0 mL) was added N′-(2-cyano-4-nitrophenyl)-N,N-dimethylformimidamide (670.5 mg, 3.07 mmol). The mixture was stirred at 80° C. for 2 h. After the reaction was completed, the resulting mixture was concentrated under vacuum. The residue was purified by flash column chromatography with CH₂Cl₂/MeOH (90/10, v/v) to afford 1-ethyl-4-([2-methyl-4-[(6-nitroquinazolin-4-yl)amino]phenyl]sulfanyl)pyridin-2-one (1.1 g, 82%) as a yellow solid. LCMS (ESI, m/z): [M+H]⁺=434.1.

### Step 5: Synthesis of 4-([4-[(6-aminoquinazolin-4-yl)amino]-2-methylphenyl]sulfanyl)-1-ethylpyridin-2-one

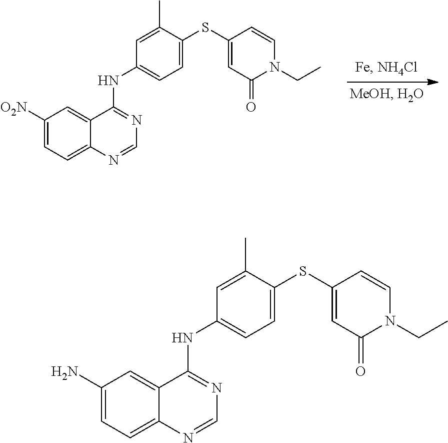 [SMILES: CCn1ccc(Sc2ccc(Nc3ncnc4ccc([N+](=O)[O-])cc34)cc2C)cc1=O ; CCn1ccc(Sc2ccc(Nc3ncnc4ccc(N)cc34)cc2C)cc1=O]

To a solution of 1-ethyl-4-([2-methyl-4-[(6-nitroquinazolin-4-yl)amino]phenyl]sulfanyl)pyridin-2-one (1.0 g, 2.31 mmol) in MeOH/H₂O (10.0 mL/3.0 mL) was added Fe (901.8 mg, 16.15 mmol) and NH₄Cl (863.8 mg, 16.15 mmol) at room temperature. The mixture was stirred at 70° C. for 3 h. After the reaction was completed, the resulting mixture was filtered. The filtrate was concentrated under vacuum. The residue was purified by flash column chromatography with CH₂Cl₂/MeOH (95/5, v/v) to afford 4-([4-[(6-aminoquinazolin-4-yl)amino]-2-methylphenyl]sulfanyl)-1-ethylpyridin-2-one (900.0 mg, 96%) as a yellow solid. LCMS (ESI, m/z): [M+H]⁺=404.1.

### Step 6: Synthesis of 3-cyclopropyl-N-[4-([4-[(1-ethyl-2-oxopyridin-4-yl)sulfanyl]-3-methylphenyl]amino)quinazolin-6-yl]prop-2-ynamide (Compound 160)

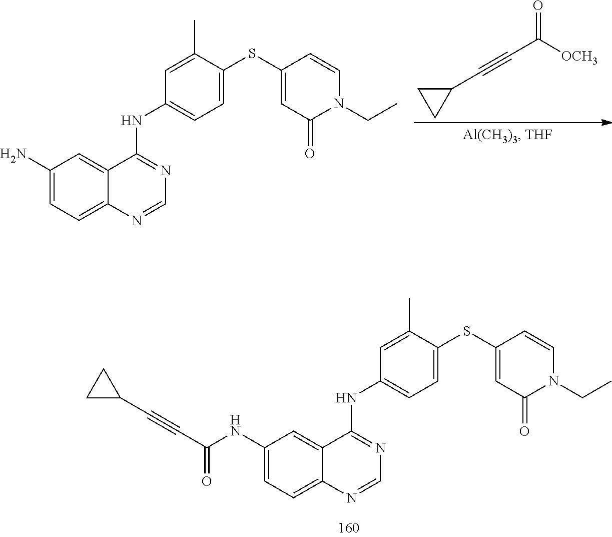

To a mixture of 4-([4-[(6-aminoquinazolin-4-yl)amino]-2-methylphenyl]sulfanyl)-1-ethylpyridin-2-one (150.0 mg, 0.37 mmol) and methyl 3-cyclopropylprop-2-ynoate (138.4 mg, 1.12 mmol) in THF (2.0 mL) was added AlMe₃ (1.2 mL, 2 mol/L) at 0° C. The mixture was stirred at 80° C. for 2 h. After the reaction was completed, the resulting mixture was quenched with water. The resulting mixture was concentrated under vacuum. The residue was purified by flash column chromatography with CH₂Cl₂/MeOH (90/10, v/v) and then purified by Prep-HPLC with the following conditions (Column: Xselect CSH OBD Column, 30×150 mm, 5 um; Mobile Phase A: Water (10 mmol/L NH₄HCO₃), Mobile Phase B: ACN; Flow rate: 60 mL/min; Gradient: 42% B to 52% B in 10 min; 254 nm) to afford 3-cyclopropyl-N-[4-([4-[(1-ethyl-2-oxopyridin-4-yl)sulfanyl]-3-methylphenyl]amino)quinazolin-6-yl]prop-2-ynamide (Compound 160) (68.3 mg, 37%) as a light yellow solid. LCMS (ESI, m/z): [M+H]⁺=496.3. ¹H NMR (300 MHz, DMSO-d₆): δ 10.88 (s, 1H), 9.97 (s, 1H), 8.73 (s, 1H), 8.62 (s, 1H), 7.96-7.91 (m, 1H), 7.88-7.78 (m, 3H), 7.62-7.55 (m, 2H), 6.06-6.03 (m, 1H), 5.57 (d, J=2.1 Hz, 1H), 3.85-3.78 (m, 2H), 2.37 (s, 3H), 1.70-1.52 (m, 1H), 1.19-1.14 (m, 3H), 1.02-0.98 (m, 2H), 0.90-0.85 (m, 2H).

### Example S51. Synthesis of 3-cyclopropyl-N-[4-([3-methyl-4-[(6-methylpyridin-3-yl)sulfanyl]phenyl]amino)quinazolin-6-yl]prop-2-ynamide (Compound 161)

### Step 1: Synthesis of 5-[[(4-methoxyphenyl)methyl]sulfanyl]-2-methylpyridine

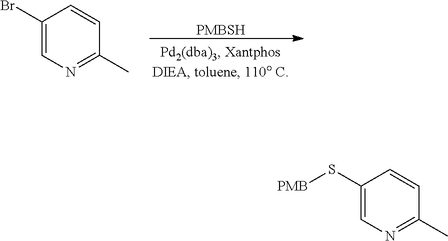

A mixture of 5-bromo-2-methylpyridine (5.0 g, 29.07 mmol), benzyl mercaptan (3.6 g, 29.07 mmol), DIEA (7.5 g, 58.13 mmol), Pd₂(dba)₃ (1.3 g, 1.45 mmol) and XantPhos (1.7 g, 2.91 mmol) in toluene (100.0 mL) was stirred at 110° C. for 3 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (96/4, v/v) to afford 5-[[(4-methoxyphenyl)methyl]sulfanyl]-2-methylpyridine (6.7 g, 93%) as a yellow oil. LCMS (ESI, m/z): [M+H]⁺=246.1.

### Step 2: Synthesis of 6-methylpyridine-3-thiol

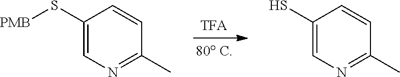

A solution of 5-[[(4-methoxyphenyl)methyl]sulfanyl]-2-methylpyridine (6.7 g, 27.31 mmol) in TFA (50.0 mL) was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by reverse phase flash column chromatography with H₂O/ACN (90/10, v/v) to afford 6-methylpyridine-3-thiol (2.2 g, 64%) as a yellow oil. LCMS (ESI, m/z): [M+H]⁺=126.0.

### Step 3: Synthesis of 2-methyl-5-[(2-methyl-4-nitrophenyl)sulfanyl]pyridine

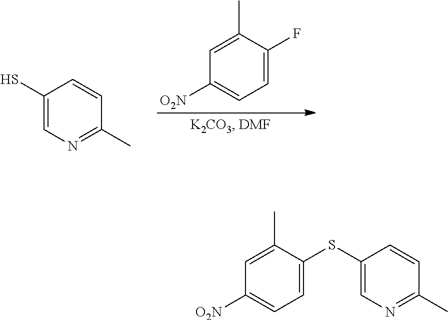

A mixture of 6-methylpyridine-3-thiol (2.2 g, 17.57 mmol), 1-fluoro-2-methyl-4-nitrobenzene (3.3 g, 21.09 mmol) and K₂CO₃ (4.9 g, 35.15 mmol) in DMF (100.0 mL) was stirred at 50° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (42/58, v/v) to afford 2-methyl-5-[(2-methyl-4-nitrophenyl)sulfanyl]pyridine (2.2 g, 48%) as a yellow solid. LCMS (ESI, m/z): [M+H]⁺=261.1.

### Step 4: Synthesis of 3-methyl-4-[(6-methylpyridin-3-yl)sulfanyl]aniline

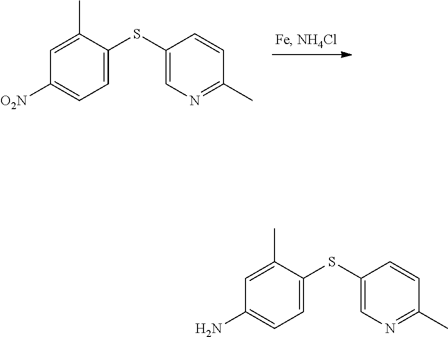 [SMILES: Cc1ccc(Sc2ccc([N+](=O)[O-])cc2C)cn1 ; Cc1ccc(Sc2ccc(N)cc2C)cn1]

A mixture of 2-methyl-5-[(2-methyl-4-nitrophenyl)sulfanyl]pyridine (2.1 g, 8.07 mmol), Fe (4.5 g, 80.71 mmol) and NH₄Cl (4.3 g, 80.71 mmol) in CH₃OH (50.0 mL) and H₂O (10.0 mL) was stirred at 80° C. for 1 h. After the reaction was completed, the resulting mixture was filtered. The filtrate was concentrated under reduced pressure. The residue was purified by flash column chromatography with CH₂Cl₂/CH₃OH (94/6, v/v) to afford 3-methyl-4-[(6-methylpyridin-3-yl)sulfanyl]aniline (1.7 g, 91%) as a yellow oil. LCMS (ESI, m/z): [M+H]⁺=231.1.

### Step 5: Synthesis of N-[3-methyl-4-[(6-methylpyridin-3-yl)sulfanyl]phenyl]-6-nitroquinazolin-4-amine

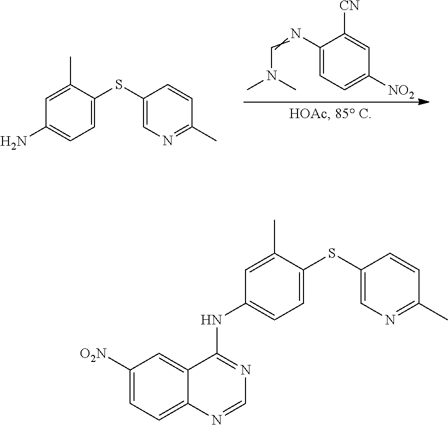 [SMILES: Cc1ccc(Sc2ccc(N)cc2C)cn1 ; CN(C)C=Nc1ccc([N+](=O)[O-])cc1C#N ; Cc1ccc(Sc2ccc(Nc3ncnc4ccc([N+](=O)[O-])cc34)cc2C)cn1]

A mixture of 3-methyl-4-[(6-methylpyridin-3-yl)sulfanyl]aniline (1.6 g, 6.95 mmol) and N′-(2-cyano-4-nitrophenyl)-N,N-dimethylformimidamide (1.8 g, 8.34 mmol) in HOAc (30.0 mL) was stirred at 85° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford N-[3-methyl-4-[(6-methylpyridin-3-yl)sulfanyl]phenyl]-6-nitroquinazolin-4-amine (1.8 g, 64%) as a yellow solid. LCMS (ESI, m/z): [M+H]⁺=404.1.

### Step 6: Synthesis of N4-[3-methyl-4-[(6-methylpyridin-3-yl)sulfanyl]phenyl]quinazoline-4,6-diamine

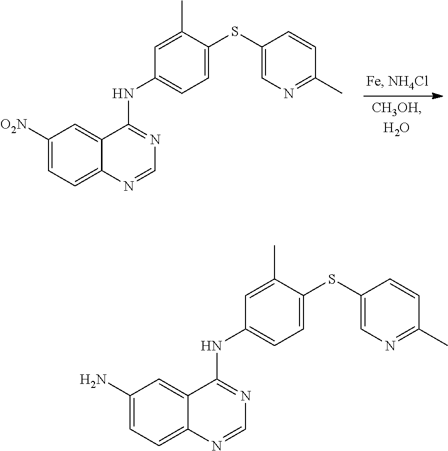 [SMILES: Cc1ccc(Sc2ccc(Nc3ncnc4ccc([N+](=O)[O-])cc34)cc2C)cn1 ; Cc1ccc(Sc2ccc(Nc3ncnc4ccc(N)cc34)cc2C)cn1]

A mixture of N-[3-methyl-4-[(6-methylpyridin-3-yl)sulfanyl]phenyl]-6-nitroquinazolin-4-amine (1.7 g, 4.21 mmol), Fe (2.4 g, 42.08 mmol) and NH₄Cl (2.25 g, 42.06 mmol) in CH₃OH (50.0 mL) and H₂O (10.0 mL) was stirred at 80° C. for 1 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford N4-[3-methyl-4-[(6-methylpyridin-3-yl)sulfanyl]phenyl]quinazoline-4,6-diamine (1.3 g, 82%) as a yellow solid. LCMS (ESI, m/z): [M+H]⁺=374.1.

### Step 7: Synthesis of 3-cyclopropyl-N-[4-([3-methyl-4-[(6-methylpyridin-3-yl)sulfanyl]phenyl]amino)quinazolin-6-yl]prop-2-ynamide (Compound 161)

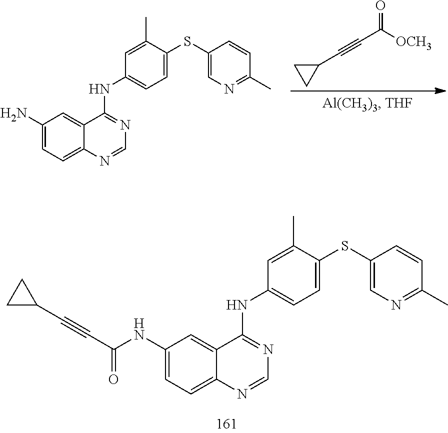

To a mixture of N4-[3-methyl-4-[(6-methylpyridin-3-yl)sulfanyl]phenyl]quinazoline-4,6-diamine (300.0 mg, 0.80 mmol) and methyl 3-cyclopropylprop-2-ynoate (299.2 mg, 2.41 mmol) in THF (10.0 mL) was added dropwise trimethylaluminium (2.1 mL, 2 mol/L) at 0° C. The resulting mixture was stirred at 80° C. for 3 h. After the reaction was completed, the reaction mixture was quenched with NH₄Cl solution at room temperature. The resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) and then purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 24% B to 33% B in 8 min; 254/220 nm) to afford 3-cyclopropyl-N-[4-([3-methyl-4-[(6-methylpyridin-3-yl)sulfanyl]phenyl]amino)quinazolin-6-yl]prop-2-ynamide (Compound 161) (69.2 mg, 18%) as a yellow solid. LCMS (ESI, m/z): [M+H]⁺=466.1. ¹H NMR (400 MHz, DMSO-d₆): δ 10.84 (s, 1H), 9.87 (s, 1H), 8.70 (s, 1H), 8.55 (s, 1H), 8.30 (d, J=2.4 Hz, 1H), 7.82-7.73 (m, 4H), 7.48-7.45 (m, 1H), 7.35 (d, J=8.4 Hz, 1H), 7.23 (d, J=8.0 Hz, 1H), 2.44 (s, 3H), 2.35 (s, 3H), 1.62-1.58 (m, 1H), 1.02-0.97 (m, 2H), 0.89-0.86 (m, 2H).

### Example S52. Synthesis of 3-cyclopropyl-N-[4-([4-[(1R)-1-[[1,2,4]triazolo[1,5-a]pyridin-7-yl]ethyl]phenyl]amino)quinazolin-6-yl]prop-2-ynamide and 3-cyclopropyl-N-[4-([4-[(1S)−1-[[1,2,4]triazolo[1,5-a]pyridin-7-yl]ethyl]phenyl]amino)quinazolin-6-yl]prop-2-ynamide (Compounds 162 and 163)

### Step 1: Synthesis of tert-butyl N-[4-(1-[[1,2,4]triazolo[1,5-a]pyridin-7-yl]ethyl)phenyl]carbamate

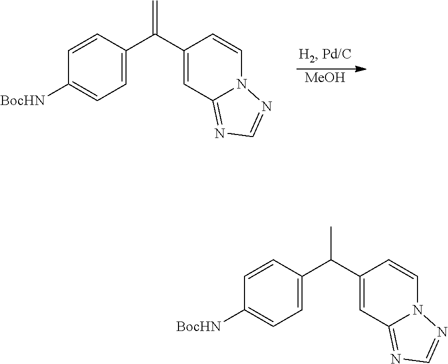 [SMILES: "MeOH" ; C=C(c1ccc(NC(=O)OC(C)(C)C)cc1)c1ccn2ncnc2c1 ; CC(c1ccc(NC(=O)OC(C)(C)C)cc1)c1ccn2ncnc2c1]

To a solution of tert-butyl N-[4-(1-[[1,2,4]triazolo[1,5-a]pyridin-7-yl]ethenyl)phenyl]carbamate (900.0 mg, 2.68 mmol) in MeOH (100.0 mL) was added Pd/C (270.0 mg, dry). The mixture was stirred at room temperature for 3 h under H₂. After the reaction was completed, the reaction mixture was filtered. The filtrate was evaporated in vacuo to afford tert-butyl N-[4-(1-[[1,2,4]triazolo[1,5-a]pyridin-7-yl]ethyl)phenyl]carbamate (750.0 mg, crude) as a light yellow solid. LCMS (ESI, m/z): [M+H]⁺=339.2.

### Step 2: Synthesis of 4-(1-[[1,2,4]triazolo[1,5-a]pyridin-7-yl]ethyl)aniline

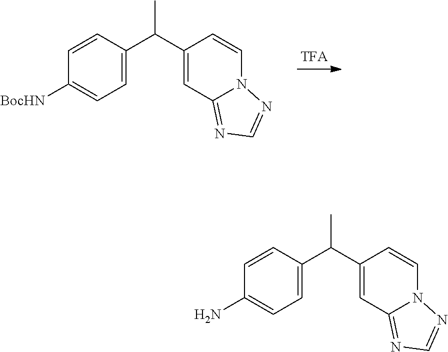 [SMILES: CC(c1ccc(NC(=O)OC(C)(C)C)cc1)c1ccn2ncnc2c1 ; CC(c1ccc(N)cc1)c1ccn2ncnc2c1]

To a solution of tert-butyl N-[4-(1-[[1,2,4]triazolo[1,5-a]pyridin-7-yl]ethyl)phenyl]carbamate (780.0 mg, crude) in DCM (20.0 mL) was added TFA (10.0 mL) at room temperature. The mixture was stirred at room temperature for 1 h. After the reaction was completed, the mixture was basified to pH=8 with saturated NaHCO₃ solution. The resulting mixture was extracted with CH₂Cl₂. The combined organic layer was washed with brine, dried over anhydrous Na₂SO₄ and filtered. The filtrate was concentrated under reduced pressure to afford 4-(1-[[1,2,4]triazolo[1,5-a]pyridin-7-yl]ethyl)aniline (540.0 mg, crude) as a yellow solid. LCMS (ESI, m/z): [M+H]⁺=239.1.

### Step 3: Synthesis of 6-nitro-N-[4-(1-[[1,2,4]triazolo[1,5-a]pyridin-7-yl]ethyl)phenyl]quinazolin-4-amine

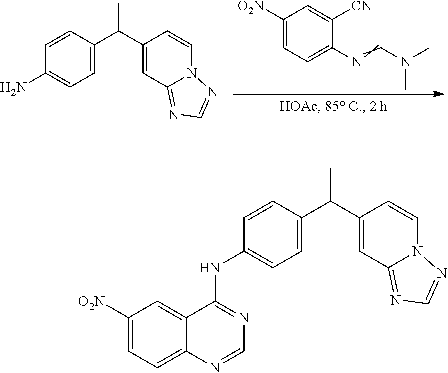 [SMILES: CC(c1ccc(N)cc1)c1ccn2ncnc2c1 ; CN(C)C=Nc1ccc([N+](=O)[O-])cc1C#N ; CC(c1ccc(Nc2ncnc3ccc([N+](=O)[O-])cc23)cc1)c1ccn2ncnc2c1]

To a mixture of 4-(1-[[1,2,4]triazolo[1,5-a]pyridin-7-yl]ethyl)aniline (540.0 mg, crude) in HOAc (10.0 mL) was added N′-(2-cyano-4-nitrophenyl)-N,N-dimethylformimidamide (494.5 mg, 2.27 mmol) at room temperature. The resulting mixture was stirred at 85° C. for 2 h. After the reaction was completed, the mixture was concentrated under vacuum. The residue was purified by flash column chromatography with CH₂Cl₂/MeOH (10/1, v/v) to afford 6-nitro-N-[4-(1-[[1,2,4]triazolo[1,5-a]pyridin-7-yl]ethyl)phenyl]quinazolin-4-amine (890.0 mg, 95%) as an orange solid. LCMS (ESI, m/z): [M+H]⁺=412.2.

### Step 4: Synthesis of N4-[4-(1-[[1,2,4]triazolo[1,5-a]pyridin-7-yl]ethyl)phenyl]quinazoline-4,6-diamine

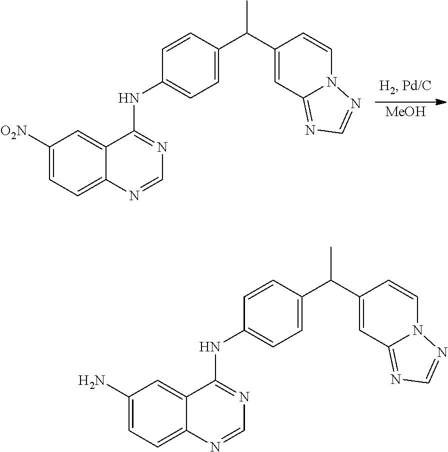 [SMILES: "MeOH" ; CC(c1ccc(Nc2ncnc3ccc([N+](=O)[O-])cc23)cc1)c1ccn2ncnc2c1 ; CC(c1ccc(Nc2ncnc3ccc(N)cc23)cc1)c1ccn2ncnc2c1]

To a solution of 6-nitro-N-[4-(1-[[1,2,4]triazolo[1,5-a]pyridin-7-yl]ethyl)phenyl]quinazolin-4-amine (890.0 mg, 2.16 mmol) in MeOH (20.0 mL) was added Pd/C (267.0 mg, dry). The mixture was stirred at room temperature for 3 h under H₂. After the reaction was completed, the reaction mixture was filtered. The filtrate was evaporated in vacuo to afford N4-[4-(1-[[1,2,4]triazolo[1,5-a]pyridin-7-yl]ethyl)phenyl]quinazoline-4,6-diamine (420.0 mg, crude) as a yellow solid. LCMS (ESI, m/z): [M+H]⁺=382.1.

### Step 5: Synthesis of 3-cyclopropyl-N-(4-[[4-(1-[[1,2,4]triazolo[1,5-a]pyridin-7-yl]ethyl)phenyl]amino]quinazolin-6-yl)prop-2-ynamide

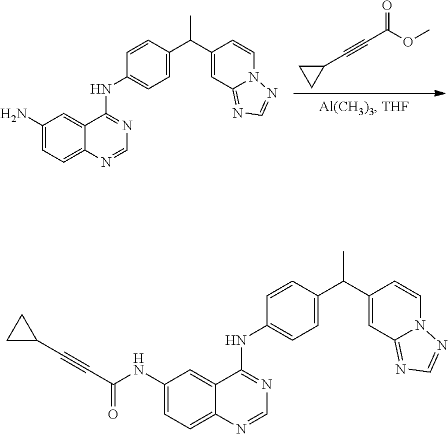

To a mixture of N4-[4-(1-[[1,2,4]triazolo[1,5-a]pyridin-7-yl]ethyl)phenyl]quinazoline-4,6-diamine (200.0 mg, crude) and methyl 3-cyclopropylprop-2-ynoate (194.3 mg, 1.57 mmol) in THF (30.0 mL) was added dropwise AlMe₃ (1.3 mL, 2 mol/L) at 0° C. The resulting mixture was stirred at 80° C. for 3 h. After the reaction was completed, the reaction mixture was quenched with sat. NH₄Cl (aq.) at room temperature. The resulting mixture was diluted with H₂O and extracted with CH₂Cl₂. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford 3-cyclopropyl-N-(4-[[4-(1-[[1,2,4]triazolo[1,5-a]pyridin-7-yl]ethyl)phenyl]amino]quinazolin-6-yl)prop-2-ynamide (200.0 mg, 46%) as a yellow solid. LCMS (ESI, m/z): [M+H]⁺=474.2.

### Step 6: Synthesis of 3-cyclopropyl-N-[4-([4-[(1R)-1-[[1,2,4]triazolo[1,5-a]pyridin-7-yl]ethyl]phenyl]amino)quinazolin-6-yl]prop-2-ynamide and 3-cyclopropyl-N-[4-([4-[(1S)-1-[[1,2,4]triazolo[1,5-a]pyridin-7-yl]ethyl]phenyl]amino)quinazolin-6-yl]prop-2-ynamide (Compounds 162 and 163)

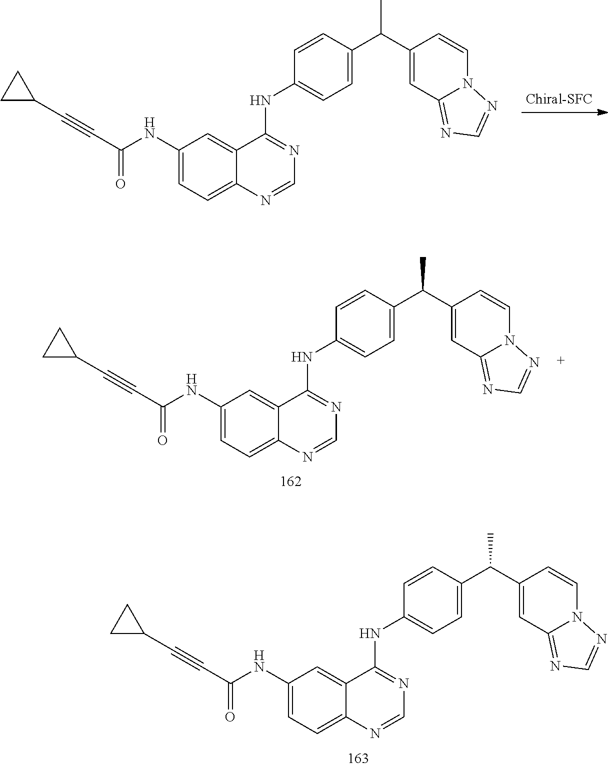 [SMILES: CC(c1ccc(Nc2ncnc3ccc(NC(=O)C#CC4CC4)cc23)cc1)c1ccn2ncnc2c1 ; C[C@H](c1ccc(Nc2ncnc3ccc(NC(=O)C#CC4CC4)cc23)cc1)c1ccn2ncnc2c1 ; C[C@@H](c1ccc(Nc2ncnc3ccc(NC(=O)C#CC4CC4)cc23)cc1)c1ccn2ncnc2c1]

The racemic 3-cyclopropyl-N-(4-[[4-(1-[[1,2,4]triazolo[1,5-a]pyridin-7-yl]ethyl)phenyl]amino]quinazolin-6-yl)prop-2-ynamide (200.0 mg) was separated by Prep-Chiral-HPLC with the following conditions (Column: CHIRALPAK IA, 2×25 cm, 5 μm; Mobile Phase A: Hex:DCM=3:1 (0.5% 2M NH₃-MeOH)-HPLC, Mobile Phase B: EtOH-HPLC; Flow rate: 20 mL/min; Gradient: 95% B to 95% B in 32 min; 220/254 nm; RT1: 21.816 min; RT2: 26.553 min) to afford 3-cyclopropyl-N-[4-([4-[1-[[1,2,4]triazolo[1,5-a]pyridin-7-yl]ethyl]phenyl]amino)quinazolin-6-yl]prop-2-ynamide Enantiomer 1 (61.0 mg, 30%) as a white solid and 3-cyclopropyl-N-[4-([4-[1-[[1,2,4]triazolo[1,5-a]pyridin-7-yl]ethyl]phenyl]amino)quinazolin-6-yl]prop-2-ynamide Enantiomer 2 (43.1 mg, 21%) as a white solid. The absolute stereochemistry of Enantiomers 1 and 2 was not assigned. The two enantiomeric structures that could be obtained from chiral separation of the enantiomeric mixture as described above are shown as Compounds 162 and 163 in Table 1.

3-cyclopropyl-N-[4-([4-[1-[[1,2,4]triazolo[1,5-a]pyridin-7-yl]ethyl]phenyl]amino)quinazolin-6-yl]prop-2-ynamide Enantiomer 1: retention time 21.816 min, LCMS (ESI, m/z): [M+H]⁺=474.3. ¹H NMR (300 MHz, DMSO-d₆): δ 10.84 (s, 1H), 9.84 (s, 1H), 8.83 (d, J=7.2 Hz, 1H), 8.66 (s, 1H), 8.48-8.45 (m, 2H), 7.82-7.70 (m, 5H), 7.38-7.34 (m, 2H), 7.09-7.06 (m, 1H), 4.38-4.31 (m, 1H), 1.67 (d, J=7.2 Hz, 3H), 1.64-1.56 (m, 1H), 1.03-1.01 (m, 2H), 0.98-0.87 (m, 2H).

3-cyclopropyl-N-[4-([4-[1-[[1,2,4]triazolo[1,5-a]pyridin-7-yl]ethyl]phenyl]amino)quinazolin-6-yl]prop-2-ynamide Enantiomer 2: retention time 26.55 min, LCMS (ESI, m/z): [M+H]⁺=474.3. ¹H NMR (300 MHz, DMSO-d₆): δ 10.84 (s, 1H), 9.83 (s, 1H), 8.83 (d, J=6.9 Hz, 1H), 8.66 (s, 1H), 8.48-8.45 (m, 2H), 7.82-7.70 (m, 5H), 7.37-7.34 (m, 2H), 7.09-7.06 (m, 1H), 4.38-4.31 (m, 1H), 1.68 (d, J=7.2 Hz, 3H), 1.64-1.55 (m, 1H), 1.03-0.97 (m, 2H), 0.92-0.87 (m, 2H). Example S53. Synthesis of (S)-3-cyclopropyl-N-(4-((4-((6-(1,2-difluoroethyl)pyridin-3-yl)oxy)-3-methylphenyl)amino)quinazolin-6-yl)propiolamide (Compound 164)

### Step 1: Synthesis of 2-bromo-5-(2-methyl-4-nitrophenoxy)pyridine

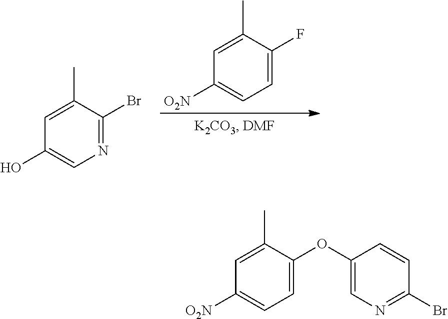

To a solution of 6-bromopyridin-3-ol (8.1 g, 46.55 mmol) in DMF (100.0 mL) was added K₂CO₃ (12.8 g, 93.11 mmol) and 1-fluoro-2-methyl-4-nitrobenzene (7.2 g, 46.55 mmol). The resulting mixture was stirred at 60° C. for 16 h. After the reaction was completed, the resulting mixture was diluted with water and extracted with ethyl acetate. The combined organic layer was washed with brine, dried over anhydrous sodium sulfate and filtered. The filtrate was concentrated under vacuum to afford 2-bromo-5-(2-methyl-4-nitrophenoxy)pyridine (13.8 g, crude) as a yellow solid. LCMS (ESI, m/z): [M+H]⁺=309.0.

### Step 2: Synthesis of 4-((6-bromopyridin-3-yl)oxy)-3-methylaniline

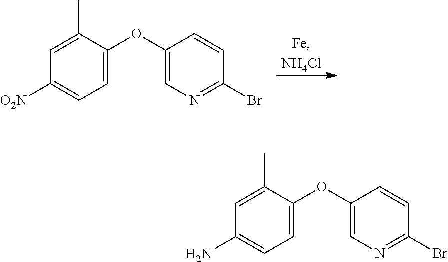

To a mixture of 2-bromo-5-(2-methyl-4-nitrophenoxy)pyridine (13.0 g, 42.05 mmol), NH₄Cl (11.2 g, 210.27 mmol) and H₂O (50.0 mL) in MeOH (200.0 mL) was added Fe (23.5 g, 420.55 mmol). The resulting mixture was stirred at 80° C. for 2 h. After the reaction was completed, the resulting mixture was filtered. The filtrate was concentrated under reduced pressure. The pH value of the residue was adjusted to 8 with NaHCO₃ solution. The mixture was extracted with ethyl acetate. The combined organic layer was washed with brine, dried over anhydrous sodium sulfate and filtered. The filtrate was concentrated under vacuum to afford 4-[(6-bromopyridin-3-yl)oxy]-3-methylaniline (11.0 g, crude) as a yellow solid. LCMS (ESI, m/z): [M+H]⁺=279.0.

### Step 3: Synthesis of N-(44(6-bromopyridin-3-yl)oxy)-3-methylphenyl)-6-nitroquinazolin-4-amine

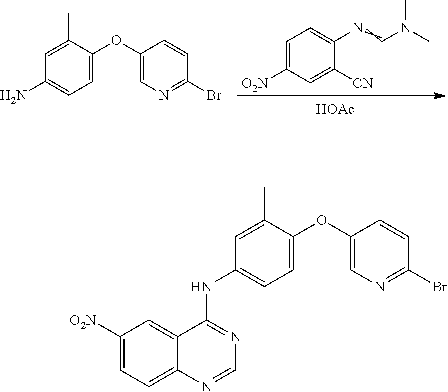 [SMILES: Cc1cc(N)ccc1Oc1ccc(Br)nc1 ; CN(C)C=Nc1ccc([N+](=O)[O-])cc1C#N ; Cc1cc(Nc2ncnc3ccc([N+](=O)[O-])cc23)ccc1Oc1ccc(Br)nc1 ; "HOAc"]

A mixture of 4-[(6-bromopyridin-3-yl)oxy]-3-methylaniline (6.0 g, crude) and N′-(2-cyano-4-nitrophenyl)-N,N-dimethylformimidamide (4.7 g, 21.49 mmol) in HOAc (20.0 mL) was stirred at 85° C. for 2 h. The resulting mixture was concentrated under vacuum. The residue was diluted with water. The pH value of the mixture was adjusted to 7 with NaHCO₃ solution. The mixture was extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (50/50, v/v) to afford N-[4-[(6-bromopyridin-3-yl)oxy]-3-methylphenyl]-6-nitroquinazolin-4-amine (8.0 g, 82%) as a yellow solid. LCMS (ESI, m/z): [M+H]⁺=452.0.

### Step 4: Synthesis of N-[4-[(6-ethenylpyridin-3-yl)oxy]-3-methylphenyl]-6-nitroquinazolin-4-amine

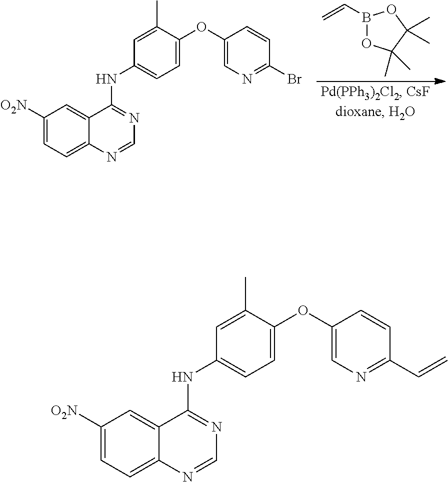

To a mixture of N-[4-[(6-bromopyridin-3-yl)oxy]-3-methylphenyl]-6-nitroquinazolin-4-amine (4.0 g, 8.84 mmol) and 2-ethenyl-4,4,5,5-tetramethyl-1,3,2-dioxaborolane (1.6 g, 10.61 mmol) in 1,4-dioxane (40.0 mL) was added Pd(PPh₃)₂Cl₂ (1.2 g, 1.77 mmol), CsF (4.0 g, 26.53 mmol) and H₂O (4.0 mL). The mixture was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (50/50, v/v) to afford N-[4-[(6-ethenylpyridin-3-yl)oxy]-3-methylphenyl]-6-nitroquinazolin-4-amine (2.2 g, 62%) as a red solid. LCMS (ESI, m/z): [M+H]⁺=400.1.

### Step 5: Synthesis of (R)-1-(5-(2-methyl-4-((6-nitroquinazolin-4-yl)amino)phenoxy)pyridin-2-yl)ethane-1,2-diol

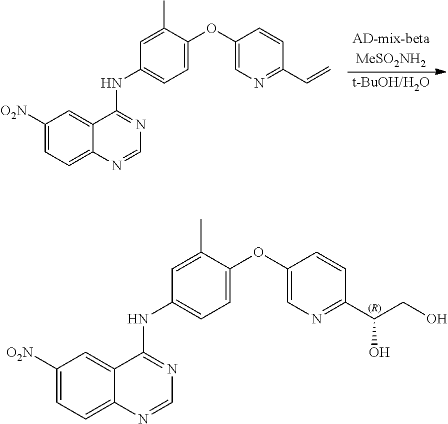

To a mixture of N-[4-[(6-ethenylpyridin-3-yl)oxy]-3-methylphenyl]-6-nitroquinazolin-4-amine (500.0 mg, 1.25 mmol) and ad-mix-beta (3.5 g, 4.51 mmol) in t-BuOH (10.0 mL) was added H₂O (10.0 mL) and methanesulfonamide (357.2 mg, 3.75 mmol). The mixture was stirred at room temperature for 16 h. After the reaction was completed, the reaction mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with MeOH/DCM (1/9, v/v) to afford (R)-1-(5-(2-methyl-4-((6-nitroquinazolin-4-yl)amino)phenoxy)pyridin-2-yl)ethane-1,2-diol (900.0 mg, 41%) as a yellow solid. LCMS (ESI, m/z): [M+H]⁺=434.1.

### Step 6: Synthesis of (S)—N-(4-((6-(1,2-difluoroethyl)pyridin-3-yl)oxy)-3-methylphenyl)-6-nitroquinazolin-4-amine

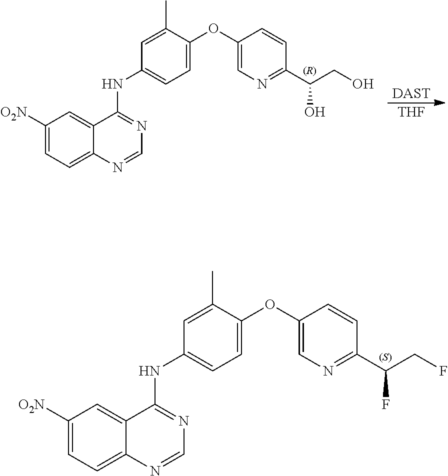

To a solution of (1R)-1-(5-(2-methyl-4-[(6-nitroquinazolin-4-yl)amino]phenoxy)pyridin-2-yl)ethane-1,2-diol (3.3 g, 7.61 mmol) in THF (50.0 mL) was added and DAST (3.7 g, 22.84 mmol). The mixture was stirred at room temperature for 16 h. The reaction was quenched with water and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (40/60, v/v) to afford (S)—N-(4-((6-(1,2-difluoroethyl)pyridin-3-yl)oxy)-3-methylphenyl)-6-nitroquinazolin-4-amine (900.0 mg, 27%) as a yellow solid. LCMS (ESI, m/z): [M+H]⁺=438.1.

### Step 7: Synthesis of (S)-N4-(4-((6-(1,2-difluoroethyl)pyridin-3-yl)oxy)-3-methylphenyl)quinazoline-4,6-diamine

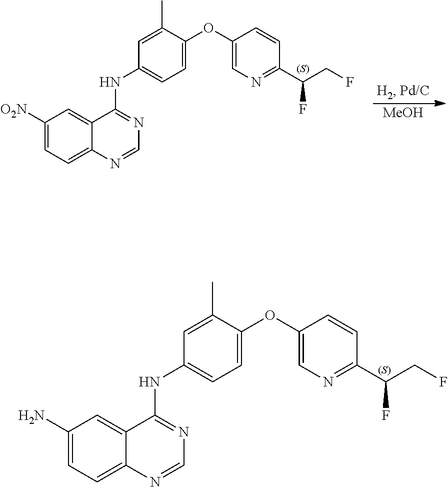

To a solution of (S)—N-(4-((6-(1,2-difluoroethyl)pyridin-3-yl)oxy)-3-methylphenyl)-6-nitroquinazolin-4-amine (900.0 mg, 2.05 mmol) in MeOH (20.0 mL) was added Pd/C (180.0 mg, dry). The mixture was stirred at room temperature for 2 h under H₂. After the reaction was completed, the resulting mixture was filtered. The filtrate was concentrated under reduced pressure to afford (S)-N4-(4-((6-(1,2-difluoroethyl)pyridin-3-yl)oxy)-3-methylphenyl)quinazoline-4,6-diamine (700.0 mg, crude) as a yellow solid. LCMS (ESI, m/z): [M+H]⁺=408.2.

### Step 8: Synthesis of (S)-3-cyclopropyl-N-(4-((4-((6-(1,2-difluoroethyl)pyridin-3-yl)oxy)-3-methylphenyl)amino)quinazolin-6-yl)propiolamide (Compound 164)

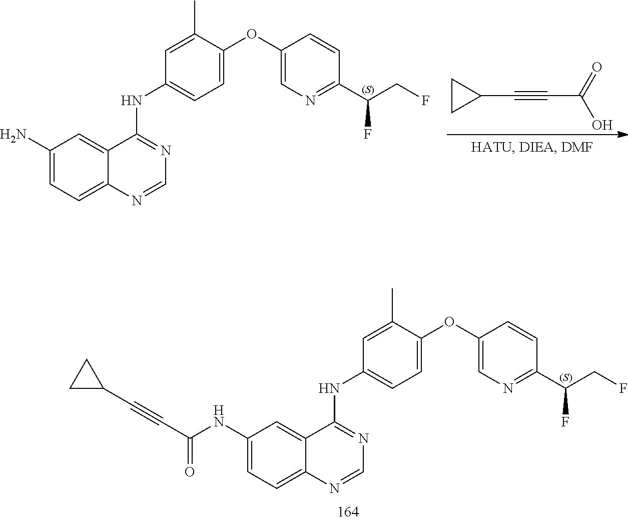

To a solution of 3-cyclopropylprop-2-ynoic acid (40.5 mg, 0.36 mmol) in DMF (5.0 mL) was added with DIEA (95.1 mg, 0.73 mmol) and HATU (140.0 mg, 0.36 mmol). The mixture was stirred at room temperature for 5 min. Then (S)-N4-(4-((6-(1,2-difluoroethyl)pyridin-3-yl)oxy)-3-methylphenyl)quinazoline-4,6-diamine (100.0 mg, crude) was added to the mixture. The resulting mixture was stirred at room temperature for 2 h.

After the reaction was completed, the reaction mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (1/9, v/v) and then purified by Prep-HPLC with the following conditions (Column: XBridge Prep OBD C18 Column, 30×150 mm, 5 um; Mobile Phase A: Water (0.1% FA), Mobile Phase B: ACN; Flow rate: 60 mL/min; Gradient: 26% B to 35% B in 7 min; 254 nm) to afford (S)-3-cyclopropyl-N-(4-((4-((6-(1,2-difluoroethyl)pyridin-3-yl)oxy)-3-methylphenyl)amino)quinazolin-6-yl)propiolamide (Compound 164) (35.8 mg, 28%) as a yellow solid. LCMS (ESI, m/z): [M+H]⁺=500.2. ¹H NMR (400 MHz, DMSO-d₆): δ 10.85 (s, 1H), 9.83 (s, 1H), 8.71 (s, 1H), 8.52 (s, 1H), 8.37 (d, J=2.8 Hz, 1H), 7.81-7.69 (m, 4H), 7.55 (d, J=8.4 Hz, 1H), 7.36-7.33 (m, 1H), 7.06 (d, J=8.8 Hz, 1H), 5.97-5.80 (m, 1H), 4.99-4.91 (m, 1H), 4.87-4.79 (m, 1H), 2.19 (s, 3H), 1.62-1.58 (m, 1H), 1.02-0.98 (m, 2H), 0.89-0.86 (m, 2H).

### Example S54. Synthesis of 3-cyclopropyl-N-[5-fluoro-4-[(2-fluoro-3-methyl-4-[[1,2,4]triazolo[1,5-a]pyridin-7-yloxy]phenyl)amino]quinazolin-6-yl]prop-2-ynamide (Compound 165)

### Step 1: Synthesis of 6-bromo-5-fluoro-N-(2-fluoro-3-methyl-4-[[1,2,4]triazolo[1,5-a]pyridin-7-yloxy]phenyl)quinazolin-4-amine

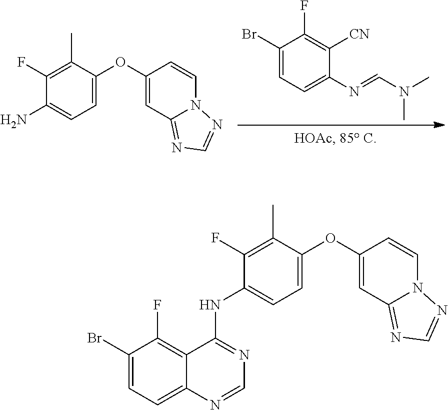 [SMILES: Cc1c(Oc2ccn3ncnc3c2)ccc(N)c1F ; CN(C)/C=N/c1ccc(Br)c(F)c1C#N ; Cc1c(Oc2ccn3ncnc3c2)ccc(Nc2ncnc3ccc(Br)c(F)c23)c1F]

To a mixture of 2-fluoro-3-methyl-4-[[1,2,4]triazolo[1,5-a]pyridin-7-yloxy]aniline (463.0 mg, 1.79 mmol) in HOAc (15.0 mL) was added (E)-N-(4-bromo-2-cyano-3-fluorophenyl)-N,N-dimethylmethanimidamide (484.2 mg, 1.79 mmol) at room temperature. The resulting mixture was stirred at 85° C. for 2 h. After the reaction was completed, the mixture was evaporated in vacuo. The residue was purified by flash column chromatography with petroleum ether/ethyl acetate (1/1, v/v) to afford 6-bromo-5-fluoro-N-(2-fluoro-3-methyl-4-[[1,2,4]triazolo[1,5-a]pyridin-7-yloxy]phenyl)quinazolin-4-amine (470.0 mg, 54%) as a white solid. LCMS (ESI, m/z): [M+H]⁺=483.2.

### Step 2: Synthesis of tert-butyl N-[5-fluoro-4-[(2-fluoro-3-methyl-4-[[1,2,4]triazolo[1,5-a]pyridin-7-yloxy]phenyl)amino]quinazolin-6-yl]carbamate

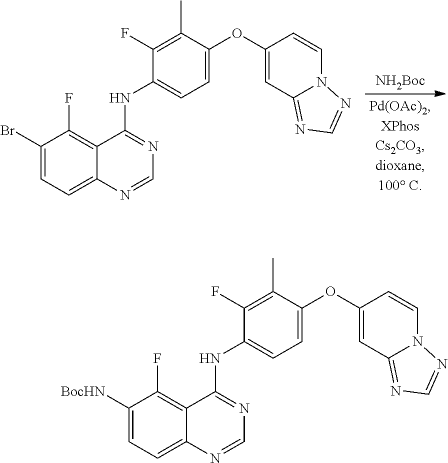 [SMILES: Cc1c(Oc2ccn3ncnc3c2)ccc(Nc2ncnc3ccc(Br)c(F)c23)c1F ; Cc1c(Oc2ccn3ncnc3c2)ccc(Nc2ncnc3ccc(NC(=O)OC(C)(C)C)c(F)c23)c1F ; "NH2Boc"]

To a solution of 6-bromo-5-fluoro-N-(2-fluoro-3-methyl-4-[[1,2,4]triazolo[1,5-a]pyridin-7-yloxy]phenyl)quinazolin-4-amine (350.0 mg, 0.72 mmol) in dioxane (7.0 mL) was added tert-butyl carbamate (254.5 mg, 2.17 mmol), Pd(OAc)₂ (32.5 mg, 0.15 mmol), Cs₂CO₃ (707.9 mg, 2.17 mmol) and XPhos (138.1 mg, 0.29 mmol) at room temperature under N₂. The resulting mixture was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (5/1, v/v) to afford tert-butyl N-[5-fluoro-4-[(2-fluoro-3-methyl-4-[[1,2,4]triazolo[1,5-a]pyridin-7-yloxy]phenyl)amino]quinazolin-6-yl]carbamate (300.0 mg, 79%) as a light yellow solid. LCMS (ESI, m/z): [M+H]⁺=520.2.

### Step 3: Synthesis of 5-fluoro-N4-(2-fluoro-3-methyl-4-[[1,2,4]triazolo[1,5-a]pyridin-7-yloxy]phenyl)quinazoline-4,6-diamine

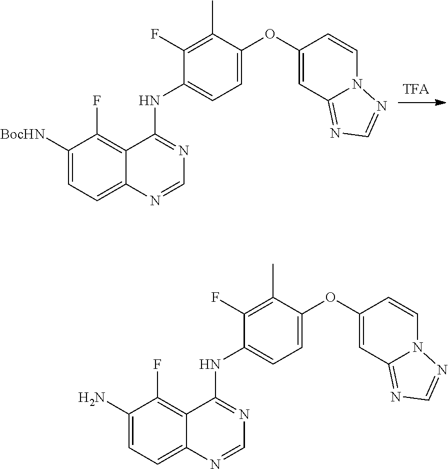 [SMILES: Cc1c(Oc2ccn3ncnc3c2)ccc(Nc2ncnc3ccc(NC(=O)OC(C)(C)C)c(F)c23)c1F ; Cc1c(Oc2ccn3ncnc3c2)ccc(Nc2ncnc3ccc(N)c(F)c23)c1F]

To a mixture of tert-butyl N-[5-fluoro-4-[(2-fluoro-3-methyl-4-[[1,2,4]triazolo[1,5-a]pyridin-7-yloxy]phenyl)amino]quinazolin-6-yl]carbamate (300.0 mg, 0.58 mmol) in DCM (10.0 mL) was added TFA (5.0 mL) at room temperature. The resulting mixture was stirred at room temperature for 1 h. After the reaction was completed, the pH value of the mixture was adjusted to 8 with saturated NaHCO₃(aq.). The resulting mixture was diluted with H₂O and extracted with DCM. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford 5-fluoro-N4-(2-fluoro-3-methyl-4-[[1,2,4]triazolo[1,5-a]pyridin-7-yloxy]phenyl)quinazoline-4,6-diamine (66.0 mg, 27%) as a yellow solid. LCMS (ESI, m/z): [M+H]⁺=420.1.

### Step 4: Synthesis of 3-cyclopropyl-N-[5-fluoro-4-[(2-fluoro-3-methyl-4-[[1,2,4]triazolo[1,5-a]pyridin-7-yloxy]phenyl)amino]quinazolin-6-yl]prop-2-ynamide (Compound 165)

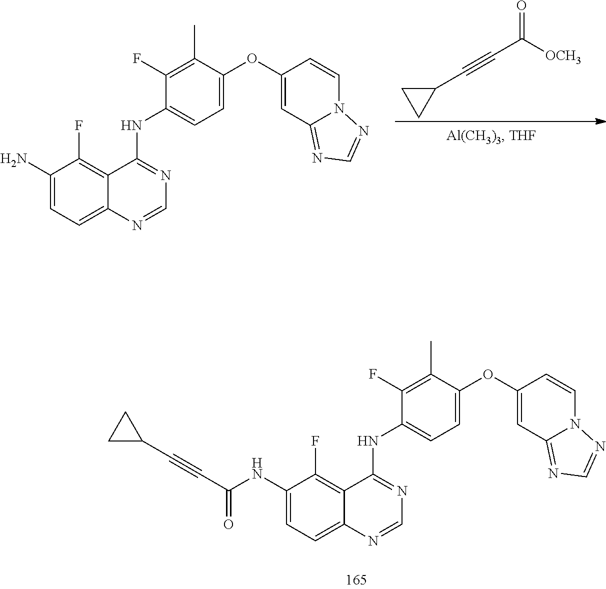

To a mixture of 5-fluoro-N4-(2-fluoro-3-methyl-4-[[1,2,4]triazolo[1,5-a]pyridin-7-yloxy]phenyl)quinazoline-4,6-diamine (66.0 mg, 0.16 mmol) and methyl 3-cyclopropylprop-2-ynoate (97.7 mg, 0.79 mmol) in THF (6.0 mL) was added AlMe₃ (0.6 mL, 2 mol/L) at 0° C. The resulting mixture was stirred at 80° C. for 3 h. After the reaction was completed, the reaction mixture was quenched with NH₄Cl (aq) at 0° C. The resulting mixture was diluted with H₂O and extracted with DCM. The combined organic layer was washed with brine, dried over anhydrous sodium sulfate and filtered. The filtrate was concentrated under reduced pressure. The residue was purified by Prep-HPLC with the following conditions: Column: Column: Xselect CSH OBD Column 30×150 mm, 5 um; Mobile Phase A: Water (0.1% FA), Mobile Phase B: ACN; Flow rate: 60 mL/min; Gradient: 34% B to 44% B in 8 min to afford 3-cyclopropyl-N-[5-fluoro-4-[(2-fluoro-3-methyl-4-[[1,2,4]triazolo[1,5-a]pyridin-7-yloxy]phenyl)amino]quinazolin-6-yl]prop-2-ynamide (Compound 165) (14.2 mg, 17%) as a light yellow solid. LCMS (ESI, m/z): [M+H]⁺=512.2. ¹H NMR (300 MHz, DMSO-d₆): δ 10.70 (s, 1H), 9.18-9.13 (m, 1H), 8.99 (d, J=7.5 Hz, 1H), 8.56 (s, 1H), 8.42 (s, 1H), 8.07-8.01 (m, 1H), 7.83-7.77 (m, 1H), 7.65 (d, J=8.7 Hz, 1H), 7.14-7.06 (m, 2H), 6.94 (d, J=2.4 Hz, 1H), 2.16 (d, J=1.8 Hz, 3H), 1.62-1.59 (m, 1H), 1.01-0.99 (m, 2H), 0.98-0.94 (m, 2H).

### Example S55. Synthesis of 3-cyclopropyl-N-[5-fluoro-4-[(2-fluoro-5-methyl-4-[[1,2,4]triazolo[1,5-a]pyridin-7-yloxy]phenyl)amino]quinazolin-6-yl]prop-2-ynamide (Compound 166)

### Step 1: Synthesis of 6-bromo-5-fluoro-N-(2-fluoro-5-methyl-4-[[1,2,4]triazolo[1,5-a]pyridin-7-yloxy]phenyl)quinazolin-4-amine

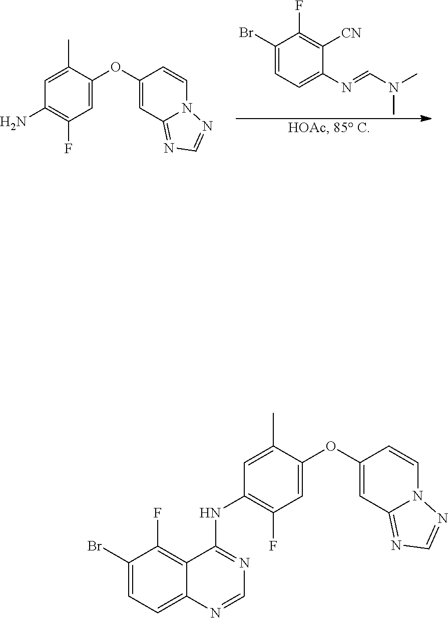 [SMILES: Cc1cc(N)c(F)cc1Oc1ccn2ncnc2c1 ; CN(C)/C=N/c1ccc(Br)c(F)c1C#N ; "HOAc, 85° C." ; Cc1cc(Nc2ncnc3ccc(Br)c(F)c23)c(F)cc1Oc1ccn2ncnc2c1]

To a mixture of 2-fluoro-5-methyl-4-[[1,2,4]triazolo[1,5-a]pyridin-7-yloxy]aniline (1.3 g, 5.03 mmol) in HOAc (40.0 mL) was added (E)-N-(4-bromo-2-cyano-3-fluorophenyl)-N,N-dimethylmethanimidamide (1.4 g, 5.03 mmol) at room temperature. The resulting mixture was stirred at 85° C. for 2 h. After the reaction was completed, the mixture was evaporated in vacuo. The residue was purified by flash column chromatography with petroleum ether/ethyl acetate (1/1, v/v) to afford 6-bromo-5-fluoro-N-(2-fluoro-5-methyl-4-[[1,2,4]triazolo[1,5-a]pyridin-7-yloxy]phenyl)quinazolin-4-amine (1.8 g, 74%) as a white solid. LCMS (ESI, m/z): [M+H]⁺=483.0.

### Step 2: Synthesis of tert-butyl N-[5-fluoro-4-[(2-fluoro-5-methyl-4-[[1,2,4]triazolo[1,5-a]pyridin-7-yloxy]phenyl)amino]quinazolin-6-yl]carbamate

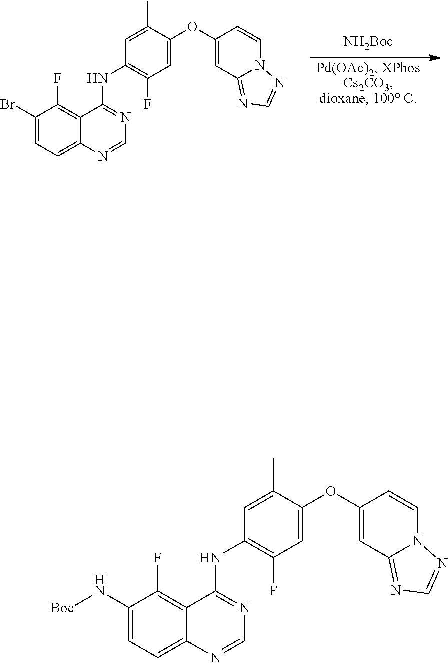 [SMILES: Cc1cc(Nc2ncnc3ccc(Br)c(F)c23)c(F)cc1Oc1ccn2ncnc2c1 ; "Pd(OAc)2, XPhosCs2CO3,dioxane, 100° C." ; Cc1cc(Nc2ncnc3ccc(NC(=O)OC(C)(C)C)c(F)c23)c(F)cc1Oc1ccn2ncnc2c1 ; "NH2Boc"]

To a solution of 6-bromo-5-fluoro-N-(2-fluoro-5-methyl-4-[[1,2,4]triazolo[1,5-a]pyridin-7-yloxy]phenyl)quinazolin-4-amine (1.0 g, 2.07 mmol) in dioxane (9.0 mL) was added tert-butyl carbamate (727.2 mg, 6.21 mmol), Cs₂CO₃ (2.0 g, 6.21 mmol), XPhos (394.6 mg, 0.83 mmol) and Pd(OAc)₂ (92.9 mg, 0.41 mmol) at room temperature under N₂. The resulting mixture was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The mixture was purified by flash column chromatography with CH₂Cl₂/MeOH (5/1, v/v) to afford tert-butyl N-[5-fluoro-4-[(2-fluoro-5-methyl-4-[[1,2,4]triazolo[1,5-a]pyridin-7-yloxy]phenyl)amino]quinazolin-6-yl]carbamate (180.0 mg, 16%) as a light yellow solid. LCMS (ESI, m/z): [M+H]⁺=520.2.

### Step 3: Synthesis of 5-fluoro-N4-(2-fluoro-5-methyl-4-[[1,2,4]triazolo[1,5-a]pyridin-7-yloxy]phenyl)quinazoline-4,6-diamine

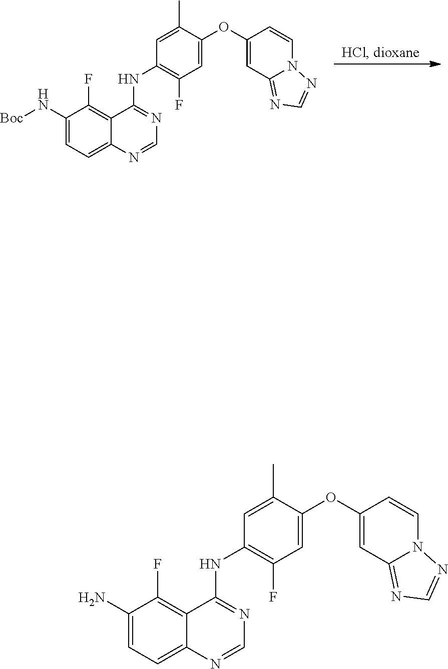 [SMILES: Cc1cc(Nc2ncnc3ccc(NC(=O)OC(C)(C)C)c(F)c23)c(F)cc1Oc1ccn2ncnc2c1 ; "HCl, dioxane" ; Cc1cc(Nc2ncnc3ccc(N)c(F)c23)c(F)cc1Oc1ccn2ncnc2c1]

To a mixture of tert-butyl N-[5-fluoro-4-[(2-fluoro-5-methyl-4-[[1,2,4]triazolo[1,5-a]pyridin-7-yloxy]phenyl)amino]quinazolin-6-yl]carbamate (180.0 mg, 0.35 mmol) in Et₂O (5.0 mL) was added HCl/dioxane (2.0 mL, 4 mol/L) at room temperature. The resulting mixture was stirred at room temperature for 1 h. After the reaction was completed, the resulting mixture was evaporated in vacuo. The pH value of the mixture was adjusted to 7 with NaHCO₃ solution. The mixture was diluted with H₂O and extracted with DCM. The combined organic layer was washed with brine, dried over anhydrous sodium sulfate and filtered. The filtrate was concentrated under reduced pressure to afford 5-fluoro-N4-(2-fluoro-5-methyl-4-[[1,2,4]triazolo[1,5-a]pyridin-7-yloxy]phenyl)quinazoline-4,6-diamine (100.0 mg, crude) as a light yellow solid. LCMS (ESI, m/z): [M+H]⁺=420.1.

### Step 4: Synthesis of 3-cyclopropyl-N-[5-fluoro-4-[(2-fluoro-5-methyl-4-[[1,2,4]triazolo[1,5-a]pyridin-7-yloxy]phenyl)amino]quinazolin-6-yl]prop-2-ynamide (Compound 166)

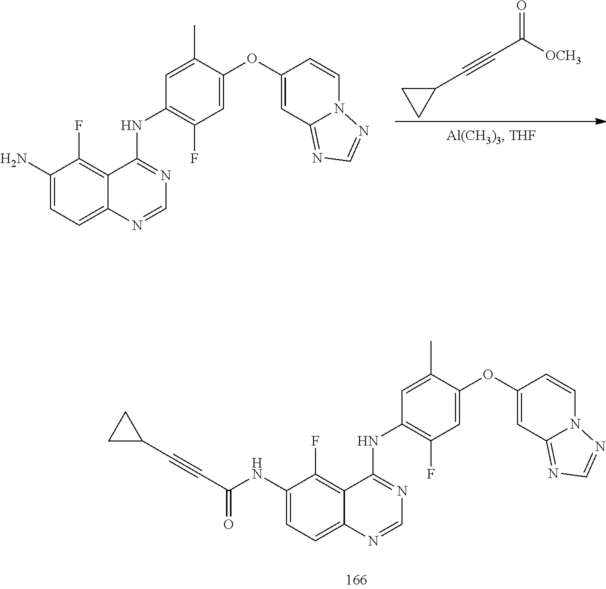

To a mixture of 5-fluoro-N4-(2-fluoro-5-methyl-4-[[1,2,4]triazolo[1,5-a]pyridin-7-yloxy]phenyl)quinazoline-4,6-diamine (100.0 mg, crude) and methyl 3-cyclopropylprop-2-ynoate (88.8 mg, 0.72 mmol) in THF (10.0 mL) was added AlMe₃ (0.6 mL, 2 mol/L) at 0° C. The resulting mixture was stirred at 80° C. for 3 h. After the reaction was completed, the reaction was quenched with NH₄Cl (aq) at 0° C. The resulting mixture was diluted with H₂O and extracted with DCM. The combined organic layer was washed with brine, dried over anhydrous sodium sulfate and filtered. The filtrate was concentrated under reduced pressure. The residue was purified by Prep-HPLC with the following conditions: Column: Xselect CSH OBD Column 30×150 mm, 5 um; Mobile Phase A: Water (0.1% FA), Mobile Phase B: ACN; Flow rate: 60 mL/min; Gradient: 33% B to 43% B in 9 min to afford 3-cyclopropyl-N-[5-fluoro-4-[(2-fluoro-5-methyl-4-[[1,2,4]triazolo[1,5-a]pyridin-7-yloxy]phenyl)amino]quinazolin-6-yl]prop-2-ynamide (Compound 166) (15.3 mg, 15%) as a white solid. LCMS (ESI, m/z): [M+H]⁺=512.1. ¹H NMR (300 MHz, DMSO-d₆): δ 10.70 (s, 1H), 9.18-9.14 (m, 1H), 8.99 (d, J=7.5 Hz, 1H), 8.56 (s, 1H), 8.43 (s, 1H), 8.06-8.01 (m, 1H), 7.85-7.82 (m, 1H), 7.65 (d, J=9.0 Hz, 1H), 7.32 (d, J=10.8 Hz, 1H), 7.09-7.05 (m, 1H), 6.93 (d, J=2.4 Hz, 1H), 2.19 (s, 3H), 1.60-1.56 (m, 1H), 1.01-0.98 (m, 2H), 0.89-0.83 (m, 2H).

### Example S56. Synthesis of 3-cyclopropyl-N-[5-fluoro-4-[(3-methyl-4-[[1,2,4]triazolo[1,5-a]pyridin-7-ylmethyl]phenyl)amino]quinazolin-6-yl]prop-2-ynamide (Compound 167)

### Step 1: Synthesis of (E)-N-(4-bromo-2-cyano-3-fluorophenyl)-N,N-dimethylmethanimidamide

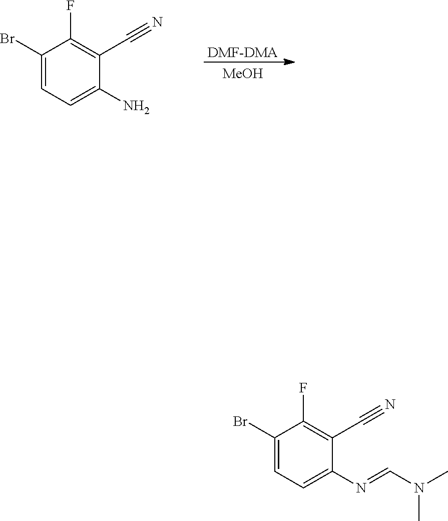

A mixture of 6-amino-3-bromo-2-fluorobenzonitrile (1.0 g, 4.65 mmol) and DMF-DMA (830.0 mg, 6.97 mmol) in MeOH (10.0 mL) was stirred at 75° C. for 3 h. After the reaction was completed, the resulting mixture was concentrated under vacuum to afford (E)-N-(4-bromo-2-cyano-3-fluorophenyl)-N,N-dimethylmethanimidamide (900.0 mg, crude) as an off-white solid. LCMS (ESI, m/z): [M+H]⁺=270.0.

### Step 2: Synthesis of 6-bromo-5-fluoro-N-(3-methyl-4-[[1,2,4]triazolo[1,5-a]pyridin-7-ylmethyl]phenyl)quinazolin-4-amine

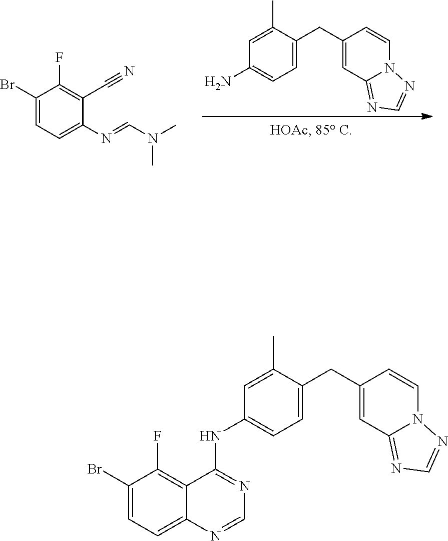 [SMILES: CN(C)/C=N/c1ccc(Br)c(F)c1C#N ; Cc1cc(N)ccc1Cc1ccn2ncnc2c1 ; "HOAc, 85° C." ; Cc1cc(Nc2ncnc3ccc(Br)c(F)c23)ccc1Cc1ccn2ncnc2c1]

To a solution of (E)-N-(4-bromo-2-cyano-3-fluorophenyl)-N,N-dimethylmethanimidamide (340.0 mg, 1.25 mmol) in HOAc (8.0 mL) was added 3-methyl-4-[[1,2,4]triazolo[1,5-a]pyridin-7-ylmethyl]aniline (300.0 mg, 1.25 mmol) at room temperature. The resulting mixture was stirred at 85° C. for 2 h. After the reaction was completed, the resulting mixture was concentrated under vacuum. The residue was purified by flash column chromatography with CH₂Cl₂/CH₃OH (9/1, v/v) to afford 6-bromo-5-fluoro-N-(3-methyl-4-[[1,2,4]triazolo[1,5-a]pyridin-7-ylmethyl]phenyl)quinazolin-4-amine (500.0 mg, 85%) as an off-white solid. LCMS (ESI, m/z): [M+H]⁺=463.0.

### Step 3: Synthesis of N6-(diphenylmethylidene)-5-fluoro-N4-(3-methyl-4-[[1,2,4]triazolo[1,5-a]pyridin-7-ylmethyl]phenyl)quinazoline-4,6-diamine

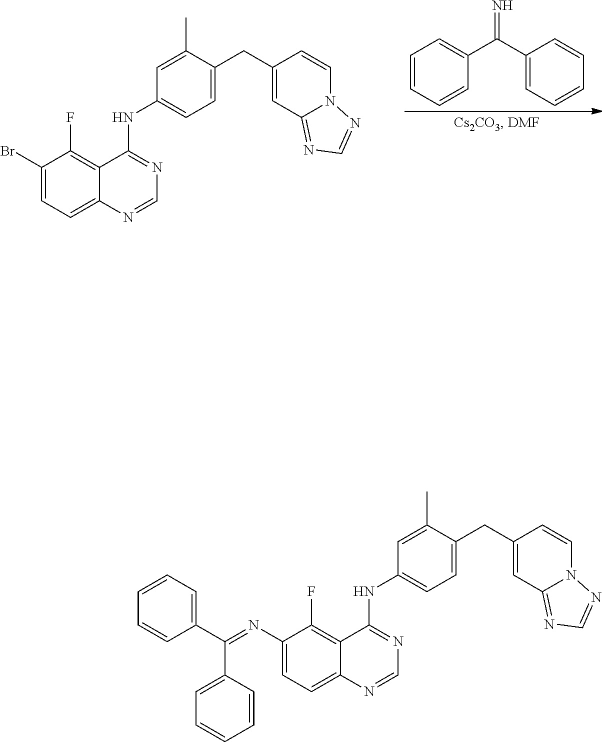

To a mixture of 6-bromo-5-fluoro-N-(3-methyl-4-[[1,2,4]triazolo[1,5-a]pyridin-7-ylmethyl]phenyl)quinazolin-4-amine (440.0 mg, 0.95 mmol) and diphenylmethanimine (258.1 mg, 1.42 mmol) in DMF (8.0 mL) was added Cs₂CO₃ (928.2 mg, 2.84 mmol), Brettphos Pd G3 (172.1 mg, 0.19 mmol) and Brettphos (203.9 mg, 0.38 mmol) at room temperature under N₂. The resulting mixture was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (10/1, v/v) to afford N6-(diphenylmethylidene)-5-fluoro-N4-(3-methyl-4-[[1,2,4]triazolo[1,5-a]pyridin-7-ylmethyl]phenyl)quinazoline-4,6-diamine (320.0 mg, 29%) as a light yellow solid. LCMS (ESI, m/z): [M+H]⁺=564.2.

### Step 4: Synthesis of 5-fluoro-N4-(3-methyl-4-[[1,2,4]triazolo[1,5-a]pyridin-7-ylmethyl]phenyl)quinazoline-4,6-diamine

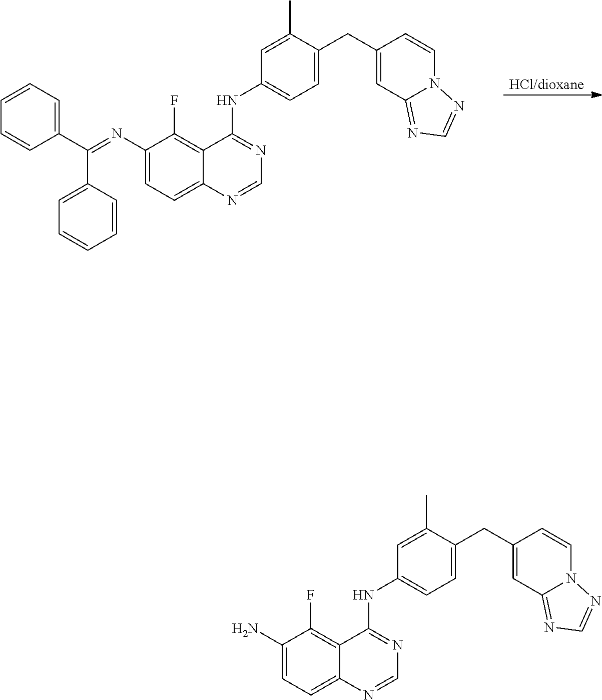 [SMILES: Cc1cc(Nc2ncnc3ccc(N=C(c4ccccc4)c4ccccc4)c(F)c23)ccc1Cc1ccn2ncnc2c1 ; "HCl/dioxane" ; Cc1cc(Nc2ncnc3ccc(N)c(F)c23)ccc1Cc1ccn2ncnc2c1]

A solution of N6-(diphenylmethylidene)-5-fluoro-N4-(3-methyl-4-[[1,2,4]triazolo[1,5-a]pyridin-7-ylmethyl]phenyl)quinazoline-4,6-diamine (300.0 mg, 0.53 mmol) in HCl/1,4-dioxane (5.0 mL, 4 mol/L) was stirred at room temperature for 2 h. The resulting mixture was concentrated under reduced pressure. The residue was purified by reverse phase flash column chromatography with acetonitrile/water (3/7, v/v) to afford 5-fluoro-N4-(3-methyl-4-[[1,2,4]triazolo[1,5-a]pyridin-7-ylmethyl]phenyl)quinazoline-4,6-diamine (48.0 mg, 22%) as a light yellow solid. LCMS (ESI, m/z): [M+H]⁺=400.2.

### Step 5: Synthesis of 3-cyclopropyl-N-[5-fluoro-4-[(3-methyl-4-[[1,2,4]triazolo[1,5-a]pyridin-7-ylmethyl]phenyl)amino]quinazolin-6-yl]prop-2-ynamide (Compound 167)

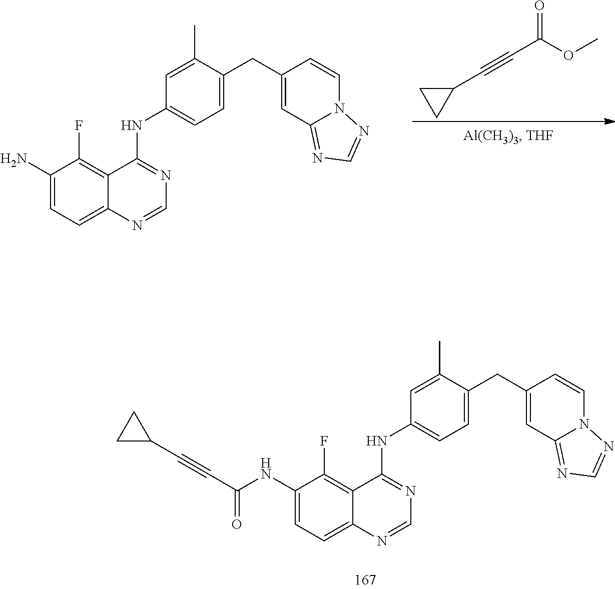

To a mixture of 5-fluoro-N4-(3-methyl-4-[[1,2,4]triazolo[1,5-a]pyridin-7-ylmethyl]phenyl)quinazoline-4,6-diamine (38.0 mg, 0.09 mmol) and methyl 3-cyclopropylprop-2-ynoate (35.4 mg, 0.28 mmol) in THF (3.0 mL) was added dropwise trimethylaluminium (0.3 mL, 2 mol/L) at 0° C. under N₂. The mixture was stirred at 80° C. for 3 h. After the reaction was completed, the reaction was quenched with sat. NH₄Cl (aq.) at room temperature. The resulting mixture was extracted with CH₂Cl₂. The combined organic layer was washed with brine, dried over anhydrous Na₂SO₄ and filtered. The filtrate was concentrated under reduced pressure. The residue was purified by flash column chromatography with CH₂Cl₂/CH₃OH (9/1, v/v) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 24% B to 54% B in 9 min; 254 nm) to afford 3-cyclopropyl-N-[5-fluoro-4-[(3-methyl-4-[[1,2,4]triazolo[1,5-a]pyridin-7-ylmethyl]phenyl)amino]quinazolin-6-yl]prop-2-ynamide (Compound 167) (4.6 mg, 9%) as a white solid. LCMS (ESI, m/z): [M+H]⁺=492.2. ¹H NMR (300 MHz, DMSO-d₆): δ 10.66 (s, 1H), 9.10-9.05 (m, 1H), 8.88 (d, J=6.9 Hz, 1H), 8.52 (s, 1H), 8.44 (s, 1H), 8.02-7.96 (m, 1H), 7.63-7.52 (m, 4H), 7.27 (d, J=7.8 Hz, 1H), 7.04-7.01 (m, 1H), 4.14 (s, 2H), 2.28 (s, 3H), 1.61-1.55 (m, 1H), 1.02-0.98 (m, 2H), 0.95-0.87 (m, 2H).

### Example S57. Synthesis of 3-cyclopropyl-N-[7-(2-hydroxyethoxy)-4-[(3-methyl-4-[[1,2,4]triazolo[1,5-a]pyridin-7-yloxy]phenyl)amino]quinazolin-6-yl]prop-2-ynamide (Compound 168)

### Step 1: Synthesis of 7-[2-(benzyloxy)ethoxy]-6-nitro-3H-quinazolin-4-one

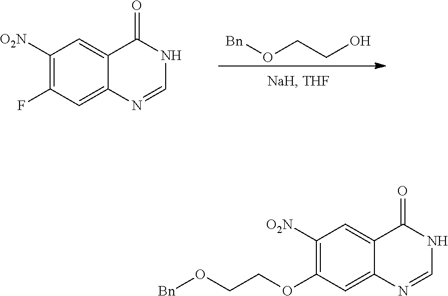

To a solution of benzyl glycol (36.4 g, 239.10 mmol) in THF (50.0 mL) was added NaH (1.7 g, 60%) at 0° C. under N₂. The mixture was stirred at 0° C. for 1 h under N₂. Then 7-fluoro-6-nitro-3H-quinazolin-4-one (5.0 g, 23.91 mmol) was added to the mixture. The mixture was stirred at 75° C. for 5 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford 7-[2-(benzyloxy)ethoxy]-6-nitro-3H-quinazolin-4-one (4.3 g, 52%) as a yellow solid. LCMS (ESI, m/z): [M+H]⁺=342.1.

### Step 2: Synthesis of 7-[2-(benzyloxy)ethoxy]-4-chloro-6-nitroquinazoline

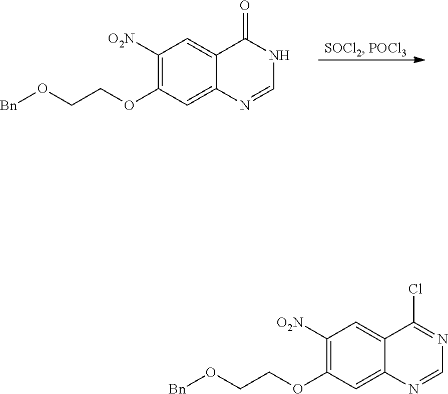

A mixture of 7-[2-(benzyloxy)ethoxy]-6-nitro-3H-quinazolin-4-one (2.0 g, 5.86 mmol) and POCl₃ (10.0 mL) in SOCl₂ (20.0 mL) was stirred at 90° C. for 2 h. After the reaction was completed, the resulting mixture was concentrated under vacuum to afford 7-[2-(benzyloxy)ethoxy]-4-chloro-6-nitroquinazoline (2.0 g, crude) as a yellow solid. LCMS (ESI, m/z): [M+H]⁺=360.1.

### Step 3: Synthesis of 7-[2-(benzyloxy)ethoxy]-N-(3-methyl-4-[[1,2,4]triazolo[1,5-a]pyridin-7-yloxy]phenyl)-6-nitroquinazolin-4-amine

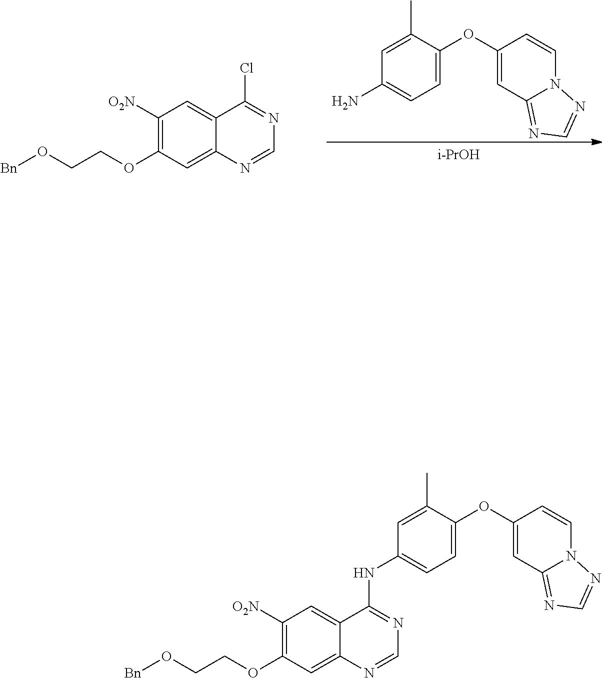 [SMILES: "i-PrOH" ; O=[N+]([O-])c1cc2c(Cl)ncnc2cc1OCCOCc1ccccc1 ; Cc1cc(N)ccc1Oc1ccn2ncnc2c1 ; Cc1cc(Nc2ncnc3cc(OCCOCc4ccccc4)c([N+](=O)[O-])cc23)ccc1Oc1ccn2ncnc2c1]

A mixture of 7-[2-(benzyloxy)ethoxy]-4-chloro-6-nitroquinazoline (2.0 g, crude) and 3-methyl-4-[[1,2,4]triazolo[1,5-a]pyridin-7-yloxy]aniline (1.3 g, 5.56 mmol) in i-PrOH (30.0 mL) was stirred at room temperature for 16 h. After the reaction was completed, the resulting mixture was concentrated under vacuum. The residue was purified by flash column chromatography with CH₂Cl₂/CH₃OH (84/16, v/v) to afford 7-[2-(benzyloxy)ethoxy]-N-(3-methyl-4-[[1,2,4]triazolo[1,5-a]pyridin-7-yloxy]phenyl)-6-nitroquinazolin-4-amine (2.2 g, 70%) as a yellow solid. LCMS (ESI, m/z): [M+H]⁺=564.2.

### Step 4: Synthesis of 2-([6-amino-4-[(3-methyl-4-[[1,2,4]triazolo[1,5-a]pyridin-7-yloxy]phenyl)amino]quinazolin-7-yl]oxy)ethanol

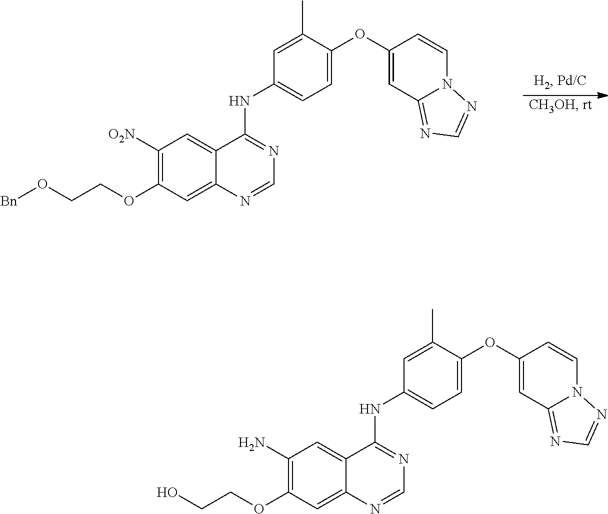 [SMILES: Cc1cc(Nc2ncnc3cc(OCCOCc4ccccc4)c([N+](=O)[O-])cc23)ccc1Oc1ccn2ncnc2c1 ; "CH3OH, rt" ; "H2, Pd/C" ; Cc1cc(Nc2ncnc3cc(OCCO)c(N)cc23)ccc1Oc1ccn2ncnc2c1]

To a solution of 7-[2-(benzyloxy)ethoxy]-N-(3-methyl-4-[[1,2,4]triazolo[1,5-a]pyridin-7-yloxy]phenyl)-6-nitroquinazolin-4-amine (2.0 g, 3.55 mmol) in CH₃OH (30.0 mL) was added Pd/C (0.6 g, dry). The mixture was stirred at room temperature for 16 h under H₂. After the reaction was completed, the resulting mixture was filtered. The filtrate was concentrated under reduced pressure. The residue was purified by flash column chromatography with CH₂Cl₂/CH₃OH (82/18, v/v) to afford 2-([6-amino-4-[(3-methyl-4-[[1,2,4]triazolo[1,5-a]pyridin-7-yloxy]phenyl)amino]quinazolin-7-yl]oxy)ethanol (330.0 mg, 20%) as a yellow oil. LCMS (ESI, m/z): [M+H]⁺=444.2.

### Step 5: Synthesis of 3-cyclopropyl-N-[7-(2-hydroxyethoxy)-4-[(3-methyl-4-[[1,2,4]triazolo[1,5-a]pyridin-7-yloxy]phenyl)amino]quinazolin-6-yl]prop-2-ynamide (Compound 168)

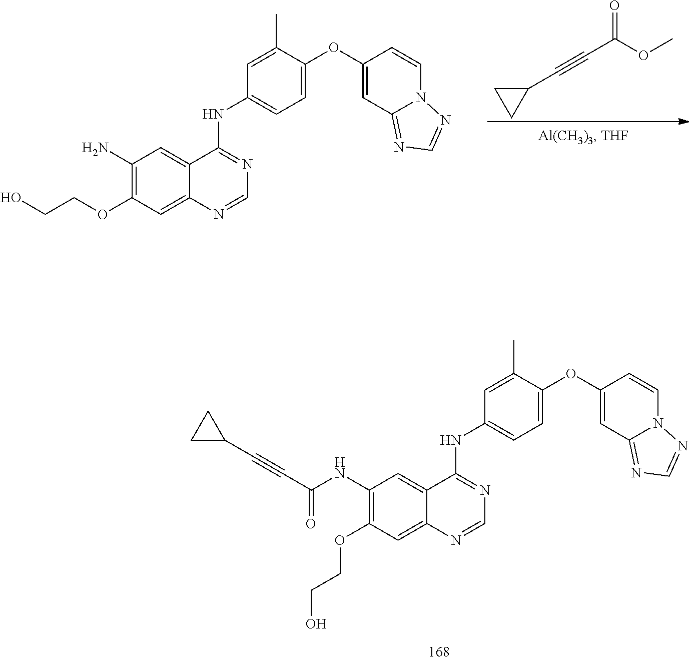

To a mixture of 2-([6-amino-4-[(3-methyl-4-[[1,2,4]triazolo[1,5-a]pyridin-7-yloxy]phenyl)amino]quinazolin-7-yl]oxy)ethanol (200.0 mg, 0.45 mmol) and methyl 3-cyclopropylprop-2-ynoate (168.0 mg, 1.35 mmol) in THF (10.0 mL) was added dropwise trimethylaluminium (1.2 mL, 2 mol/L) at 0° C. The resulting mixture was stirred at 80° C. for 3 h. After the reaction was completed, the reaction mixture was quenched with NH₄Cl solution and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (89/11, v/v) and then purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 18% B to 28% B in 9 min; 254/220 nm) to afford 3-cyclopropyl-N-[7-(2-hydroxyethoxy)-4-[(3-methyl-4-[[1,2,4]triazolo[1,5-a]pyridin-7-yloxy]phenyl)amino]quinazolin-6-yl]prop-2-ynamide (Compound 168) (2.0 mg, 1%) as a white solid. LCMS (ESI, m/z): [M+H]⁺=536.3.41 NMR (300 MHz, DMSO-d₆): δ 10.02 (s, 1H), 9.75 (s, 1H), 8.94 (d, J=7.5 Hz, 1H), 8.82 (s, 1H), 8.52 (s, 1H), 8.39 (s, 1H), 7.82-7.78 (m, 2H), 7.27 (s, 1H), 7.21-7.16 (m, 1H), 7.05-7.02 (m, 1H), 6.80 (d, J=2.4 Hz, 1H), 5.10 (br, 1H), 4.23-4.20 (m, 2H), 3.83-3.77 (m, 2H), 2.19 (s, 3H), 1.69-1.59 (m, 1H), 1.13-0.80 (m, 4H).

### Example S58. Synthesis of 3-cyclopropyl-N-(4-(4-(1-ethyl-2-oxo-1,2-dihydropyridin-4-yloxy)-3-methylphenylamino)quinazolin-6-yl)propiolamide (Compound 169)

### Step 1: Synthesis of 3-cyclopropyl-N-(4-(4-(1-ethyl-2-oxo-1,2-dihydropyridin-4-yloxy)-3-methylphenylamino)quinazolin-6-yl)propiolamide (Compound 169)

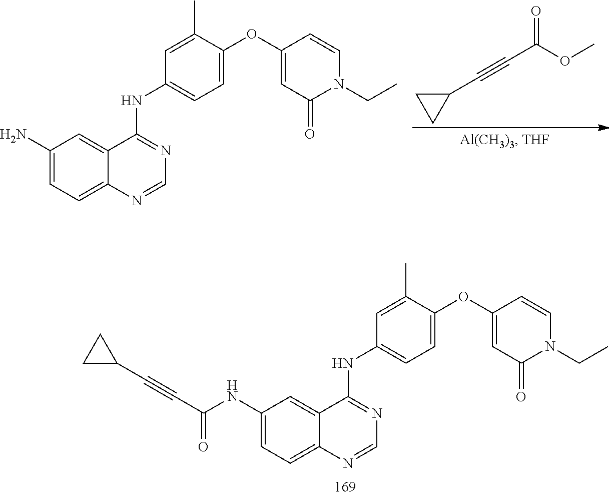

To a mixture of 4-[4-[(6-aminoquinazolin-4-yl)amino]-2-methylphenoxy]-1-ethylpyridin-2-one (200.0 mg, 0.52 mmol) and methyl 3-cyclopropylprop-2-ynoate (192.2 mg, 1.55 mmol) in THF (5.0 mL) was added AlMe₃ (1.4 mL, 2.80 mmol) at 0° C. The resulting mixture was stirred at 80° C. for 3 h. After the reaction was completed, the reaction was quenched with sat. NH₄Cl (aq.) at room temperature and extracted with CH₂Cl₂. The combined organic layer was washed with brine, dried over anhydrous Na₂SO₄ and filtered. The filtrate was concentrated under reduced pressure. The residue was purified by flash column chromatography with CH₂Cl₂/CH₃OH (9/1, v/v) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 23% B to 53% B in 9 min; 254 nm) to afford 3-cyclopropyl-N-[4-([4-[(1-ethyl-2-oxopyridin-4-yl)oxy]-3-methylphenyl]amino)quinazolin-6-yl]prop-2-ynamide (Compound 169) (71.4 mg, 28%) as a light yellow solid. LCMS (ESI, m/z): [M+H]⁺=480.2. ¹H NMR (300 MHz, DMSO-d₆): δ 10.86 (s, 1H), 9.86 (s, 1H), 8.71 (s, 1H), 8.54 (s, 1H), 7.82-7.73 (m, 5H), 7.12 (d, J=8.4 Hz, 1H), 6.13-6.10 (m, 1H), 5.33 (d, J=2.7 Hz, 1H), 3.89-3.82 (m, 2H), 2.14 (s, 3H), 1.63-1.58 (m, 1H), 1.21-1.16 (m, 3H), 1.03-0.98 (m, 2H), 0.89-0.80 (m, 2H).

### Example S59. Synthesis of 3-cyclopropyl-N-{7-[2-(dimethylamino)ethoxy]-4-[(3-methyl-4-{[1,2,4]triazolo[1,5-a]pyridin-7-yloxy}phenyl)amino]quinazolin-6-yl}prop-2-ynamide hydrochloride (Compound 170)

### Step 1: Synthesis of 3-cyclopropyl-N-{7-[2-(dimethylamino)ethoxy]-4-[(3-methyl-4-{[1,2,4]triazolo[1,5-a]pyridin-7-yloxy}phenyl)amino]quinazolin-6-yl}prop-2-ynamide hydrochloride (Compound 170)

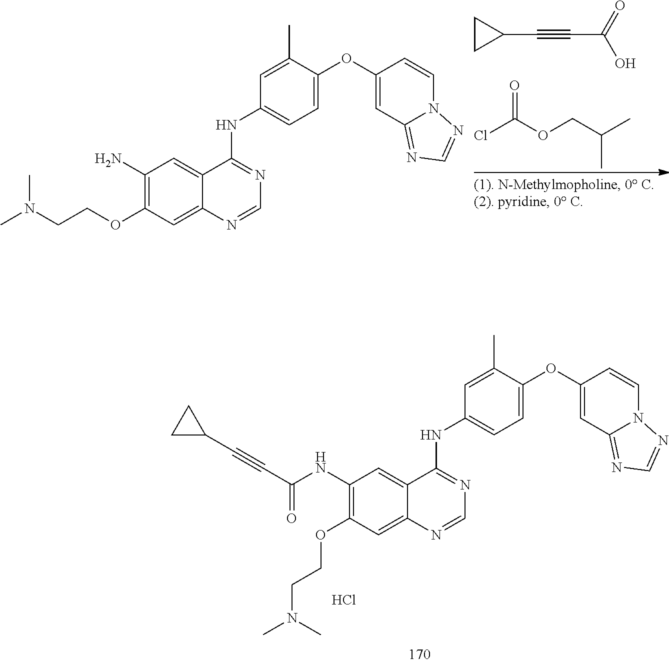

To a solution of 3-cyclopropylprop-2-ynoic acid (617.8 mg, 5.61 mmol) in THF (12.0 mL) was added 2-methylpropyl carbonochloridate (1010.1 mg, 7.39 mmol) at 0° C. under N₂. The resulting mixture was stirred at 0° C. for 5 min. Then N-methylmorpholine (85.9 mg, 0.85 mmol) was added dropwise to the mixture at 0° C. The mixture was stirred at 0° C. for another 10 min. Then a solution of 7-[2-(dimethylamino)ethoxy]-N4-(3-methyl-4-{[1,2,4]triazolo[1,5-a]pyridin-7-yloxy}phenyl)quinazoline-4,6-diamine (600.0 mg, 1.27 mmol) in pyridine (8.0 mL) was added dropwise to the mixture at 0 C. The mixture was stirred at 0° C. for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions: Column: Xselect CSH OBD Column 30×150 mm, 5 um; Mobile Phase A: Water (0.05% HCl), Mobile Phase B: ACN; Flow rate: 60 mL/min; Gradient: 5% B to 30% B in 8 min, 254/220 nm; RTI: 7 min to afford 3-cyclopropyl-N-{7-[2-(dimethylamino)ethoxy]-4-[(3-methyl-4-{[1,2,4]triazolo[1,5-a]pyridin-7-yloxy}phenyl)amino]quinazolin-6-yl}prop-2-ynamide hydrochloride (Compound 170) (256.9 mg, 33%) as a yellow solid. LCMS (ESI, m/z): [M+H^{]}+=563.2. ¹H NMR (300 MHz, DMSO-d₆): δ 11.50 (s, 1H), 11.40 (s, 1H), 10.80 (s, 1H), 9.11 (s, 1H), 9.02 (d, J=7.5 Hz, 1H), 8.92 (s, 1H), 8.52 (s, 1H), 7.71 (d, J=2.4 Hz, 1H), 7.66-7.62 (m, 1H), 7.54 (s, 1H), 7.31 (d, J=8.7 Hz, 1H), 7.13-7.10 (m, 1H), 6.88 (d, J=2.7 Hz, 1H), 4.62-4.58 (m, 2H), 3.70-3.65 (m, 2H), 2.88 (s, 3H), 2.86 (s, 3H), 2.23 (s, 3H), 1.59-1.50 (m, 1H), 0.99-0.92 (m, 4H).

### Example S60. Synthesis of 3-cyclopropyl-N-[4-({2-fluoro-3-methyl-4-[(6-methylpyridin-3-yl)oxy]phenyl}amino)quinazolin-6-yl]prop-2-ynamide (Compound 171)

### Step 1: Synthesis of N-{2-fluoro-3-methyl-4-[(6-methylpyridin-3-yl)oxy]phenyl}-6-nitroquinazolin-4-amine

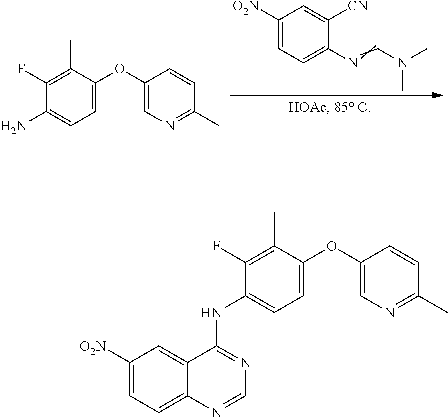 [SMILES: Cc1ccc(Oc2ccc(N)c(F)c2C)cn1 ; CN(C)C=Nc1ccc([N+](=O)[O-])cc1C#N ; Cc1ccc(Oc2ccc(Nc3ncnc4ccc([N+](=O)[O-])cc34)c(F)c2C)cn1]

To a mixture of 2-fluoro-3-methyl-4-[(6-methylpyridin-3-yl)oxy]aniline (500.0 mg, 2.15 mmol) in HOAc (10.0 mL) was added N′-(2-cyano-4-nitrophenyl)-N,N-dimethylformimidamide (469.8 mg, 2.15 mmol) at room temperature. The resulting mixture was stirred at 85° C. for 2 h. After the reaction was completed, the reaction mixture was evaporated in vacuo. The residue was purified by flash column chromatography with CH₂Cl₂/MeOH (1/1, v/v) to afford N-{2-fluoro-3-methyl-4-[(6-methylpyridin-3-yl)oxy]phenyl}-6-nitroquinazolin-4-amine (850.0 mg, 97%) as a yellow oil. LCMS (ESI, m/z): [M+H]⁺=406.1.

### Step 2: Synthesis of N4-{2-fluoro-3-methyl-4-[(6-methylpyridin-3-yl)oxy]phenyl}quinazoline-4,6-diamine

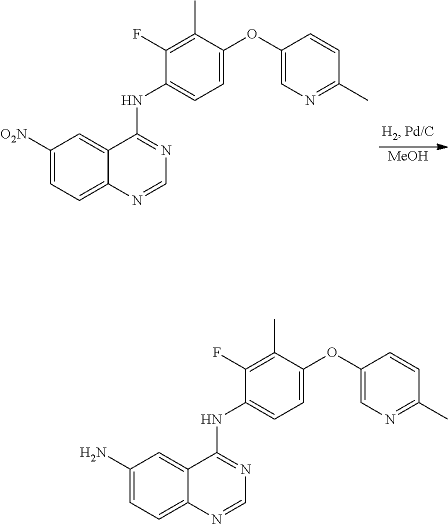 [SMILES: "MeOH" ; Cc1ccc(Oc2ccc(Nc3ncnc4ccc([N+](=O)[O-])cc34)c(F)c2C)cn1 ; Cc1ccc(Oc2ccc(Nc3ncnc4ccc(N)cc34)c(F)c2C)cn1]

To a solution of N-{2-fluoro-3-methyl-4-[(6-methylpyridin-3-yl)oxy]phenyl}-6-nitroquinazolin-4-amine (850.0 mg, 2.10 mmol) in MeOH (20.0 mL) was added Pd/C (255.0 mg, dry) at room temperature. The mixture was stirred at room temperature for 3 h under H₂. After the reaction was completed, the reaction mixture was filtered. The filtrate was evaporated in vacuo to afford N4-{2-fluoro-3-methyl-4-[(6-methylpyridin-3-yl)oxy]phenyl}quinazoline-4,6-diamine (750.0 mg, crude) as a yellow oil. LCMS (ESI, m/z): [M+H]⁺=376.1.

### Step 3: Synthesis of 3-cyclopropyl-N-[4-({2-fluoro-3-methyl-4-[(6-methylpyridin-3-yl)oxy]phenyl}amino)quinazolin-6-yl]prop-2-ynamide (Compound 171)

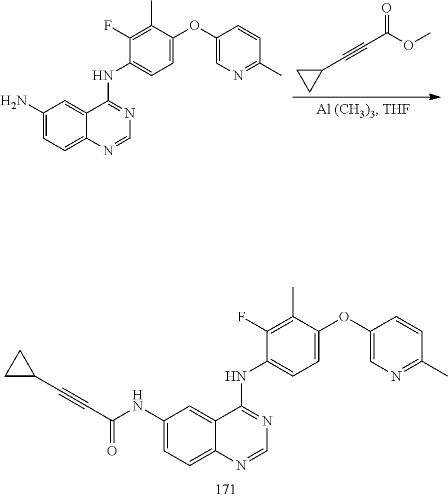

To a mixture of N4-{2-fluoro-3-methyl-4-[(6-methylpyridin-3-yl)oxy]phenyl}quinazoline-4,6-diamine (200.0 mg, 0.53 mmol) and methyl 3-cyclopropylprop-2-ynoate (198.4 mg, 1.60 mmol) in THF (10.0 mL) was added AlMe₃ (1.3 mL, 2 mol/L) at 0° C. The resulting mixture was stirred at 80° C. for 3 h. After the reaction was completed, the reaction mixture was quenched with NH₄Cl (aq) at 0° C. and then diluted with H₂O. The resulting mixture was extracted with DCM. The combined organic layer was washed with brine, dried over anhydrous sodium sulfate and filtered. The filtrate was concentrated under reduced pressure. The residue was purified by Prep-HPLC with the following conditions: (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 29% B to 57% B in 9 min, 254/220 nm) to afford 3-cyclopropyl-N-[4-({2-fluoro-3-methyl-4-[(6-methylpyridin-3-yl)oxy]phenyl}amino)quinazolin-6-yl]prop-2-ynamide (Compound 171) (65.6 mg, 26%) as a light yellow solid. LCMS (ESI, m/z): [M+H]⁺=468.1. ¹H NMR (400 MHz, DMSO-d₆): δ 10.87 (s, 1H), 9.83 (s, 1H), 8.70 (s, 1H), 8.44 (s, 1H), 8.26 (d, J=2.4 Hz, 1H), 7.81-7.74 (m, 2H), 7.37-7.29 (m, 3H), 6.75 (d, J=8.8 Hz, 1H), 2.47 (s, 3H), 2.19 (s, 3H), 1.62-1.59 (m, 1H), 1.02-0.97 (m, 2H), 0.90-0.85 (m, 2H).

### Example S61. Synthesis of 3-cyclopropyl-N-[4-([2-fluoro-5-methyl-4-[(6-methylpyridin-3-yl)oxy]phenyl]amino)quinazolin-6-yl]prop-2-ynamide (Compound 172)

### Step 1: Synthesis of 5-(5-fluoro-2-methyl-4-nitrophenoxy)-2-methylpyridine

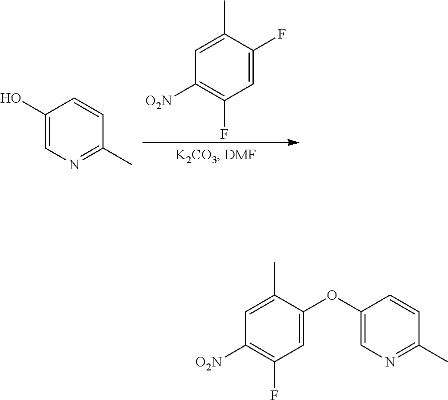

To a solution of 6-methylpyridin-3-ol (1.0 g, 9.16 mmol) in DMF (40.0 mL) was added 1,5-difluoro-2-methyl-4-nitrobenzene (3.2 g, 18.33 mmol) and K₂CO₃ (7.6 g, 54.98 mmol) at room temperature. The resulting mixture was stirred at room temperature for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford 5-(5-fluoro-2-methyl-4-nitrophenoxy)-2-methylpyridine (540.0 mg, 22%) as a colorless oil. LCMS (ESI, m/z): [M+H]⁺=263.1.

### Step 2: Synthesis of 2-fluoro-5-methyl-4-[(6-methylpyridin-3-yl)oxy]aniline

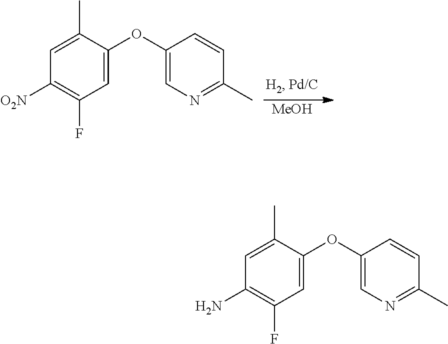 [SMILES: "MeOH" ; Cc1ccc(Oc2cc(F)c([N+](=O)[O-])cc2C)cn1 ; Cc1ccc(Oc2cc(F)c(N)cc2C)cn1]

To a solution of 5-(5-fluoro-2-methyl-4-nitrophenoxy)-2-methylpyridine 540.0 mg, 2.06 mmol) in MeOH (100.0 mL) was added Pd/C (162.0 mg, dry). The mixture was stirred at room temperature for 4 h under H₂. After the reaction was completed, the reaction mixture was filtered. The filtrate was evaporated in vacuo to afford 2-fluoro-5-methyl-4-[(6-methylpyridin-3-yl)oxy]aniline (500.0 mg, crude) as a light yellow solid. LCMS (ESI, m/z): [M+H]⁺=233.1.

### Step 3: Synthesis of N-[2-fluoro-5-methyl-4-[(6-methylpyridin-3-yl)oxy]phenyl]-6-nitroquinazolin-4-amine

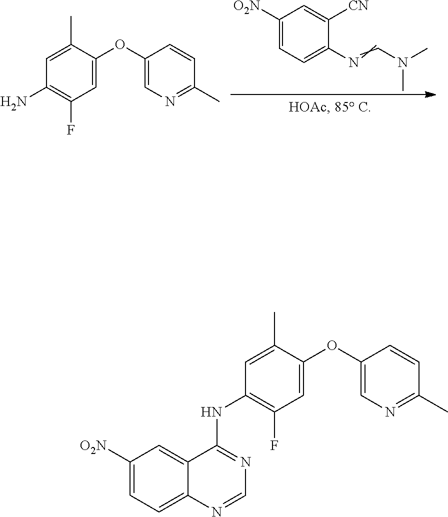 [SMILES: Cc1ccc(Oc2cc(F)c(N)cc2C)cn1 ; CN(C)C=Nc1ccc([N+](=O)[O-])cc1C#N ; Cc1ccc(Oc2cc(F)c(Nc3ncnc4ccc([N+](=O)[O-])cc34)cc2C)cn1]

To a mixture of 2-fluoro-5-methyl-4-[(6-methylpyridin-3-yl)oxy]aniline (450.0 mg, crude) in HOAc (15.0 mL) was added N′-(2-cyano-4-nitrophenyl)-N,N-dimethylformimidamide (422.8 mg, 1.94 mmol) at room temperature. The resulting mixture was stirred at 85° C. for 2 h. After the reaction was completed, the reaction mixture was evaporated in vacuo. The residue was purified by flash column chromatography with petroleum ether/ethyl acetate (1/10, v/v) to afford N-[2-fluoro-5-methyl-4-[(6-methylpyridin-3-yl)oxy]phenyl]-6-nitroquinazolin-4-amine (600.0 mg, 76%) as a light yellow solid. LCMS (ESI, m/z): [M+H]⁺=406.1.

### Step 4: Synthesis of N4-[2-fluoro-5-methyl-4-[(6-methylpyridin-3-yl)oxy]phenyl]quinazoline-4,6-diamine

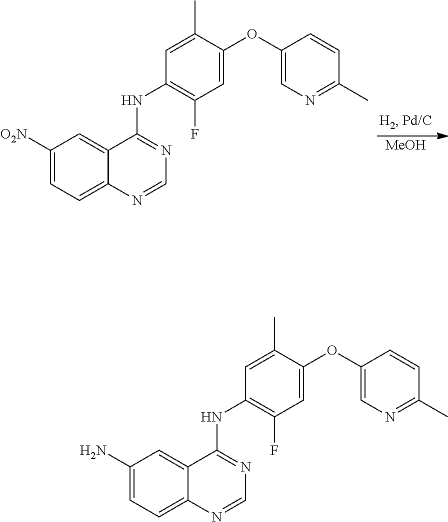 [SMILES: "MeOH" ; Cc1ccc(Oc2cc(F)c(Nc3ncnc4ccc([N+](=O)[O-])cc34)cc2C)cn1 ; Cc1ccc(Oc2cc(F)c(Nc3ncnc4ccc(N)cc34)cc2C)cn1]

To a solution of N-[2-fluoro-5-methyl-4-[(6-methylpyridin-3-yl)oxy]phenyl]-6-nitroquinazolin-4-amine (550.0 mg, 1.36 mmol) in MeOH (30.0 mL) was added Pd/C (164.6 mg, dry). The mixture was stirred at room temperature for 4 h under H₂. After the reaction was completed, the reaction mixture was filtered. The filtrate was evaporated in vacuo to afford N4-[2-fluoro-5-methyl-4-[(6-methylpyridin-3-yl)oxy]phenyl]quinazoline-4,6-diamine (500.0 mg, crude) as a light yellow solid. LCMS (ESI, m/z): [M+H]⁺=376.1.

### Step 5: Synthesis of 3-cyclopropyl-N-[4-([2-fluoro-5-methyl-4-[(6-methylpyridin-3-yl)oxy]phenyl]amino)quinazolin-6-yl]prop-2-ynamide (Compound 172)

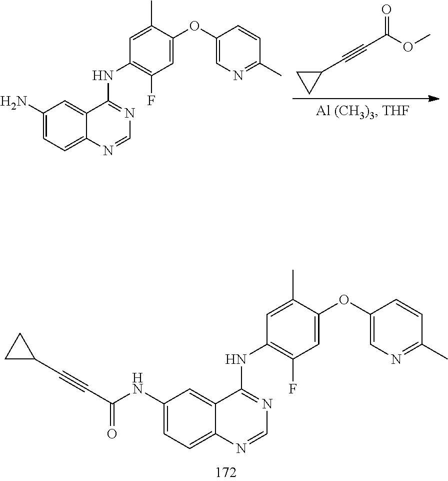

To a mixture of N4-[2-fluoro-5-methyl-4-[(6-methylpyridin-3-yl)oxy]phenyl]quinazoline-4,6-diamine (200.0 mg, crude) and methyl 3-cyclopropylprop-2-ynoate (198.4 mg, 1.60 mmol) in THF (10.0 mL) was added AlMe₃ (1.3 mL, 2 mol/L) at 0° C. The resulting mixture was stirred at 80° C. for 3 h. After the reaction was completed, the reaction mixture was quenched with NH₄Cl (aq) at 0° C. The resulting mixture was diluted with H₂O and extracted with DCM. The combined organic layer was washed with brine, dried over anhydrous sodium sulfate and filtered. The filtrate was concentrated under reduced pressure. The residue was purified by Prep-HPLC with the following conditions: Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 41% B to 51% B in 10 min, 254/220 nm; to afford 3-cyclopropyl-N-[4-([2-fluoro-5-methyl-4-[(6-methylpyridin-3-yl)oxy]phenyl]amino)quinazolin-6-yl]prop-2-ynamide (Compound 172) (86.4 mg, 34%) as a light yellow solid. LCMS (ESI, m/z): [M+H]⁺=468.1. ¹H NMR (300 MHz, CD₃OD): δ 8.66 (d, J=1.8 Hz, 1H), 8.42 (s, 1H), 8.23 (d, J=2.4 Hz, 1H), 7.83-7.76 (m, 2H), 7.53 (d, J=8.7 Hz, 1H), 7.43-7.39 (m, 1H), 7.33 (d, J=8.4 Hz, 1H), 6.85 (d, J=10.8 Hz, 1H), 2.55 (s, 3H), 2.27 (s, 3H), 1.59-1.50 (m, 1H), 1.05-1.00 (m, 2H), 0.95-0.91 (m, 2H).

### Example S62. Synthesis of 3-cyclopropyl-N-(5-fluoro-4-(3-methyl-4-(6-methylpyridin-3-yloxy)phenylamino)quinazolin-6-yl)propiolamide (Compound 173)

### Step 1: Synthesis of 2-methyl-5-(2-methyl-4-nitrophenoxy)pyridine

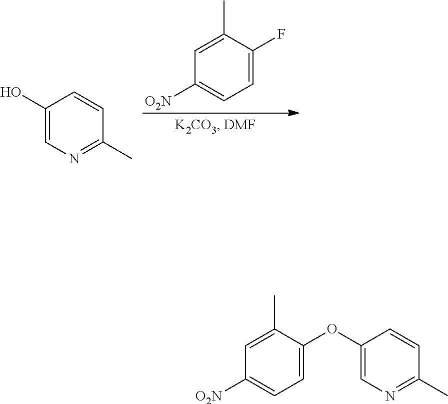

To a mixture of 6-methylpyridin-3-ol (5.0 g, 45.81 mmol) and 1-fluoro-2-methyl-4-nitrobenzene (8.5 g, 54.98 mmol) in DMF (50.0 mL) was added K₂CO₃ (12.6 g, 91.63 mmol) at room temperature. The resulting mixture was stirred at 50° C. for 16 h. After the reaction was completed, the resulting mixture was diluted with H₂O and extracted with ethyl acetate. The combined organic layer was washed with brine, dried over anhydrous Na₂SO₄ and filtered. The filtrate was concentrated under reduced pressure to afford 2-methyl-5-(2-methyl-4-nitrophenoxy)pyridine (10.0 g, crude) as a yellow oil. LCMS (ESI, m/z): [M+H]⁺=245.1.

### Step 2: Synthesis of 3-methyl-4-[(6-methylpyridin-3-yl)oxy]aniline

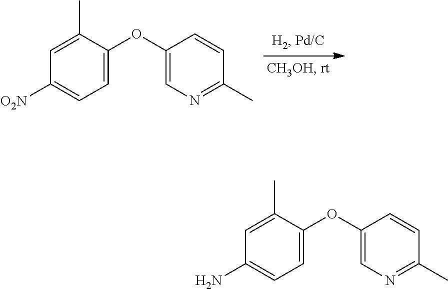 [SMILES: Cc1ccc(Oc2ccc([N+](=O)[O-])cc2C)cn1 ; Cc1ccc(Oc2ccc(N)cc2C)cn1]

To a solution of 2-methyl-5-(2-methyl-4-nitrophenoxy)pyridine (10.0 g, crude) in CH₃OH (100.0 mL) were added Pd/C (3.3 g, dry) at room temperature. The resulting mixture was stirred at room temperature for 3 h under H₂. After the reaction was completed, the resulting mixture was filtered. The filtrate was concentrated under reduced pressure afford 3-methyl-4-[(6-methylpyridin-3-yl)oxy]aniline (10.0 g, crude) as an off-white solid. LCMS (ESI, m/z): [M+H]⁺=215.1.

### Step 3: Synthesis of 6-bromo-5-fluoro-N-[3-methyl-4-[(6-methylpyridin-3-yl)oxy]phenyl]quinazolin-4-amine

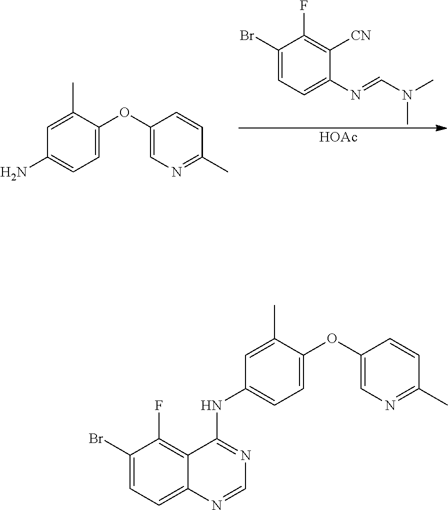 [SMILES: Cc1ccc(Oc2ccc(N)cc2C)cn1 ; CN(C)/C=N/c1ccc(Br)c(F)c1C#N ; Cc1ccc(Oc2ccc(Nc3ncnc4ccc(Br)c(F)c34)cc2C)cn1 ; "HOAc"]

A mixture of 3-methyl-4-[(6-methylpyridin-3-yl)oxy]aniline (2.4 g, crude) and (E)-N-(4-bromo-2-cyano-3-fluorophenyl)-N,N-dimethylmethanimidamide (2.0 g, 7.41 mmol) in HOAc (40.0 mL) was stirred at 85° C. for 2 h. After the reaction was completed, the resulting mixture was concentrated under reduced pressure. The residue was purified by flash column chromatography with CH₂Cl₂/CH₃OH (10/1, v/v) to afford 6-bromo-5-fluoro-N-[3-methyl-4-[(6-methylpyridin-3-yl)oxy]phenyl]quinazolin-4-amine (4.7 g, 95%) as a green oil. LCMS (ESI, m/z): [M+H]⁺=439.1.

### Step 4: Synthesis of tert-butyl N-[5-fluoro-4-([3-methyl-4-[(6-methylpyridin-3-yl)oxy]phenyl]amino)quinazolin-6-yl]carbamate

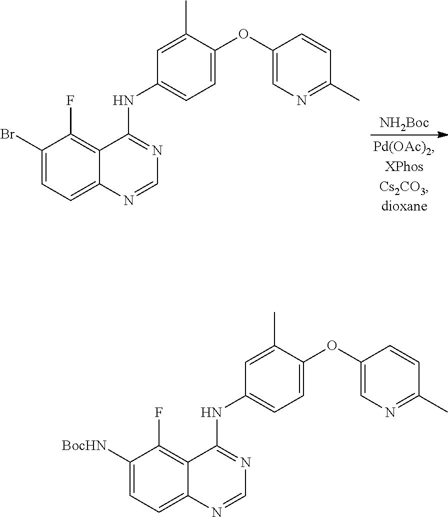 [SMILES: Cc1ccc(Oc2ccc(Nc3ncnc4ccc(Br)c(F)c34)cc2C)cn1 ; Cc1ccc(Oc2ccc(Nc3ncnc4ccc(NC(=O)OC(C)(C)C)c(F)c34)cc2C)cn1 ; "NH2Boc"]

A mixture of 6-bromo-5-fluoro-N-[3-methyl-4-[(6-methylpyridin-3-yl)oxy]phenyl]quinazolin-4-amine (4.0 g, 9.11 mmol), tert-butyl carbamate (3.2 g, 27.32 mmol), Pd(OAc)₂ (0.4 g, 1.82 mmol), XPhos (1.7 g, 3.64 mmol) and Cs₂CO₃ (8.9 g, 27.32 mmol) in 1,4-dioxane (40.0 mL) was stirred at 100° C. for 4 h under N₂. After the reaction was completed, the resulting mixture was dilute with water and extracted with ethyl acetate. The combined organic layer was washed with brine, dried over anhydrous Na₂SO₄ and filtered. The filtrate was concentrated under reduced pressure. The residue was purified by flash column chromatography with petroleum ether/ethyl acetate (1/1, v/v) to afford tert-butyl N-[5-fluoro-4-([3-methyl-4-[(6-methylpyridin-3-yl)oxy]phenyl]amino)quinazolin-6-yl]carbamate (800.0 mg, 18%) as a yellow solid. LCMS (ESI, m/z): [M+H]⁺=476.2.

### Step 5: Synthesis of 5-fluoro-N4-(3-methyl-4-(6-methylpyridin-3-yloxy)phenyl)quinazoline-4,6-diamine

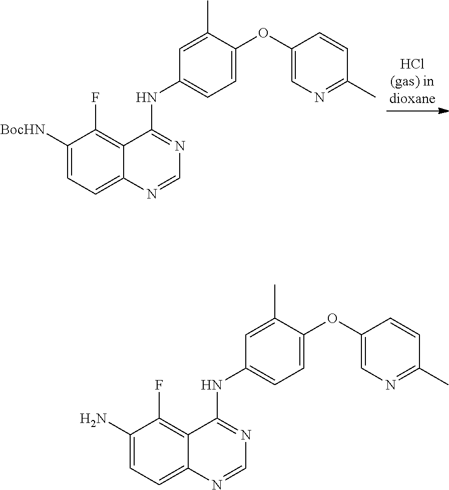 [SMILES: Cc1ccc(Oc2ccc(Nc3ncnc4ccc(NC(=O)OC(C)(C)C)c(F)c34)cc2C)cn1 ; Cc1ccc(Oc2ccc(Nc3ncnc4ccc(N)c(F)c34)cc2C)cn1]

A mixture of tert-butyl N-[5-fluoro-4-([3-methyl-4-[(6-methylpyridin-3-yl)oxy]phenyl]amino)quinazolin-6-yl]carbamate (800.0 mg, 0.21 mmol) in/dioxane (10.0 mL, 4 mol/L) was stirred at room temperature for 1 h. After the reaction was completed, the resulting mixture was concentrated under reduced pressure. The residue was purified by flash column chromatography with CH₂Cl₂/CH₃OH (10/1, v/v) to afford 5-fluoro-N4-(3-methyl-4-(6-methylpyridin-3-yloxy)phenyl)quinazoline-4,6-diamine (430.0 mg, 68%) as a white solid. LCMS (ESI, m/z): [M+H]⁺=376.3.

### Step 6: Synthesis of 3-cyclopropyl-N-(5-fluoro-4-(3-methyl-4-(6-methylpyridin-3-yloxy)phenylamino)quinazolin-6-yl)propiolamide (Compound 173)

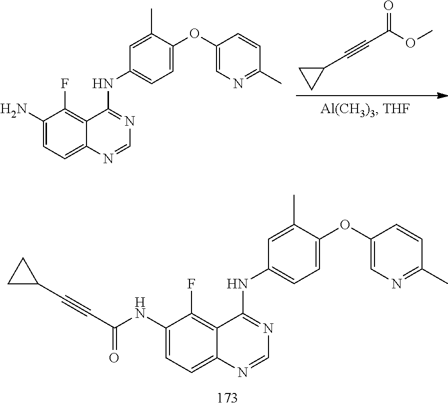

To a mixture of 5-fluoro-N4-(3-methyl-4-(6-methylpyridin-3-yloxy)phenyl)quinazoline-4,6-diamine (200.0 mg, 0.53 mmol) and methyl 3-cyclopropylprop-2-ynoate (198.4 mg, 1.60 mmol) in THF (8.0 mL) was added dropwise AlMe₃ (1.3 mL, 2 mol/L) at 0° C. The mixture was stirred at 80° C. for 3 h. After the reaction was completed, the reaction mixture was quenched with saturated NH₄Cl solution and then extracted with CH₂Cl₂. The combined organic layer was washed with brine, dried over anhydrous sodium sulfate and filtered. The filtrate was concentrated under vacuum. The residue was purified by flash column chromatography with CH₂Cl₂/CH₃OH (10/1, v/v) and then purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 16% B to 30% B in 9 min, 254 nm) to afford 3-cyclopropyl-N-(5-fluoro-4-(3-methyl-4-(6-methylpyridin-3-yloxy)phenylamino)quinazolin-6-yl)propiolamide (Compound 173) (73.9 mg, 29%) as a yellow solid. LCMS (ESI, m/z): [M+H]⁺=468.2. ¹H NMR (300 MHz, DMSO-d₆): δ 10.64 (s, 1H), 9.12-9.07 (m, 1H), 8.51 (s, 1H), 8.18-8.17 (m, 1H), 8.00-7.94 (m, 1H), 7.65-7.53 (m, 3H), 7.25-7.18 (m, 2H), 6.94 (d, J=8.7 Hz, 1H), 2.43 (s, 3H), 2.19 (s, 3H), 1.62-1.53 (m, 1H), 0.98-0.80 (m, 4H).

### Example S63. Synthesis of 3-cyclopropyl-N-[7-[3-(dimethylamino)propoxy]-4-[(3-methyl-4-[[1,2,4]triazolo[1,5-a]pyridin-7-yloxy]phenyl)amino]quinazolin-6-yl]prop-2-ynamide (Compound 174)

### Step 1: Synthesis of 7-[3-(dimethylamino)propoxy]-6-nitro-3H-quinazolin-4-one

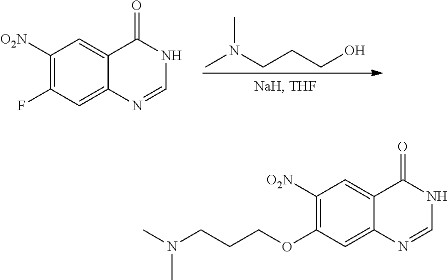

To a solution of 3-(dimethylamino)propan-1-ol (14.8 g, 143.45 mmol) in THF (10.0 mL) was added NaH (1.0 g, 60%) at 0° C. under N₂. The mixture was stirred at 0° C. for 1 h. Then 7-fluoro-6-nitro-3H-quinazolin-4-one (3.0 g, 14.36 mmol) was added to the mixture. The mixture was stirred at 75° C. for 5 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by reverse phase flash column chromatography with H₂O/CH₃CN (80/20, v/v) to afford 7-[3-(dimethylamino)propoxy]-6-nitro-3H-quinazolin-4-one (2.8 g, 66%) as a brown yellow solid. LCMS (ESI, m/z): [M+H]⁺=293.1.

### Step 2: Synthesis of [3-[(4-chloro-6-nitroquinazolin-7-yl)oxy]propyl]dimethylamine

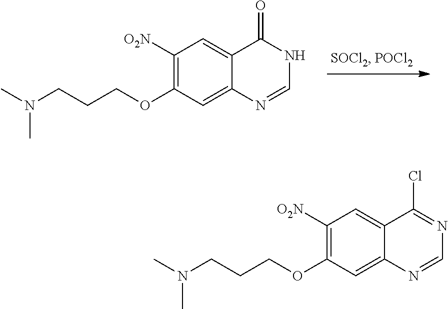 [SMILES: CN(C)CCCOc1cc2nc[nH]c(=O)c2cc1[N+](=O)[O-] ; CN(C)CCCOc1cc2ncnc(Cl)c2cc1[N+](=O)[O-]]

A mixture of 7-[3-(dimethylamino)propoxy]-6-nitro-3H-quinazolin-4-one (2.0 g, 6.84 mmol), DMF (0.1 g, 0.001 mmol) and POCl₃ (10.0 mL) in SOCl₂ (10.0 mL) was stirred at 90° C. for 2 h. After the reaction was completed, the resulting mixture was concentrated under vacuum to afford [3-[(4-chloro-6-nitroquinazolin-7-yl)oxy]propyl]dimethylamine (2.0 g, crude) as yellow solid. LCMS (ESI, m/z): [M+H]⁺=311.1.

### Step 3: Synthesis of 7-[3-(dimethylamino)propoxy]-N-(3-methyl-4-[[1,2,4]triazolo[1,5-a]pyridin-7-yloxy]phenyl)-6-nitroquinazolin-4-amine

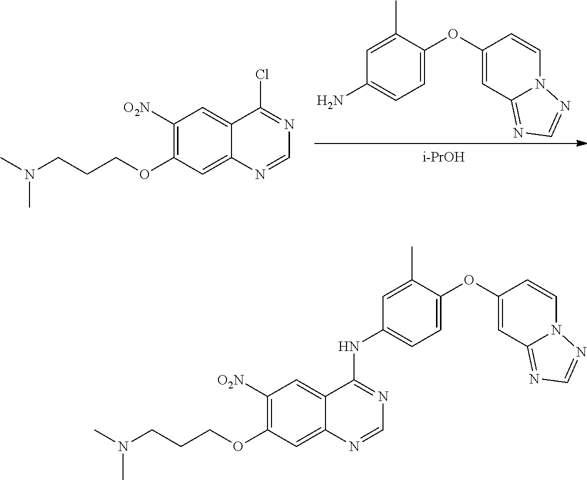 [SMILES: "i-PrOH" ; CN(C)CCCOc1cc2ncnc(Cl)c2cc1[N+](=O)[O-] ; Cc1cc(N)ccc1Oc1ccn2ncnc2c1 ; Cc1cc(Nc2ncnc3cc(OCCCN(C)C)c([N+](=O)[O-])cc23)ccc1Oc1ccn2ncnc2c1]

A mixture of [3-[(4-chloro-6-nitroquinazolin-7-yl)oxy]propyl]dimethylamine (2.0 g, crude) and 3-methyl-4-[[1,2,4]triazolo[1,5-a]pyridin-7-yloxy]aniline (1.6 g, 6.47 mmol) in i-PrOH (30.0 mL) was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford 7-[3-(dimethylamino)propoxy]-N-(3-methyl-4-[[1,2,4]triazolo[1,5-a]pyridin-7-yloxy]phenyl)-6-nitroquinazolin-4-amine (700.0 mg, 21%) as a yellow solid. LCMS (ESI, m/z): [M+H]⁺=515.2.

### Step 4: Synthesis of 7-[3-(dimethylamino)propoxy]-N4-(3-methyl-4-[[1,2,4]triazolo[1,5-a]pyridin-7-yloxy]phenyl)quinazoline-4,6-diamine

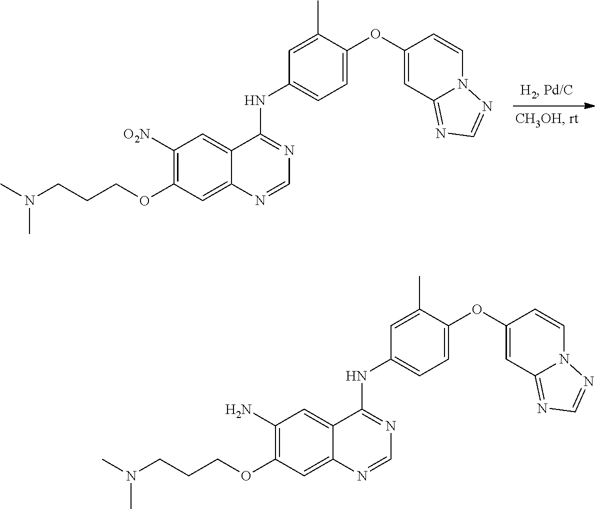 [SMILES: Cc1cc(Nc2ncnc3cc(OCCCN(C)C)c([N+](=O)[O-])cc23)ccc1Oc1ccn2ncnc2c1 ; Cc1cc(Nc2ncnc3cc(OCCCN(C)C)c(N)cc23)ccc1Oc1ccn2ncnc2c1]

To a solution of 7-[3-(dimethylamino)propoxy]-N-(3-methyl-4-[[1,2,4]triazolo[1,5-a]pyridin-7-yloxy]phenyl)-6-nitroquinazolin-4-amine (700.0 mg, 1.36 mmol) in CH₃OH (35.0 mL) was added Pd/C (209.9 mg, dry). The mixture was stirred at room temperature for 3 h under H₂. After the reaction was completed, the resulting mixture was filtered. The filtrate was concentrated under reduced pressure. The residue was purified by flash column chromatography with CH₂Cl₂/CH₃OH (94/6, v/v) to afford 7-[3-(dimethylamino)propoxy]-N4-(3-methyl-4-[[1,2,4]triazolo[1,5-a]pyridin-7-yloxy]phenyl)quinazoline-4,6-diamine (420.0 mg, 63%) as a yellow solid. LCMS (ESI, m/z): [M+H]⁺=485.2.

### Step 5: Synthesis of 3-cyclopropyl-N-[7-[3-(dimethylamino)propoxy]-4-[(3-methyl-4-[[1,2,4]triazolo[1,5-a]pyridin-7-yloxy]phenyl)amino]quinazolin-6-yl]prop-2-ynamide (Compound 174)

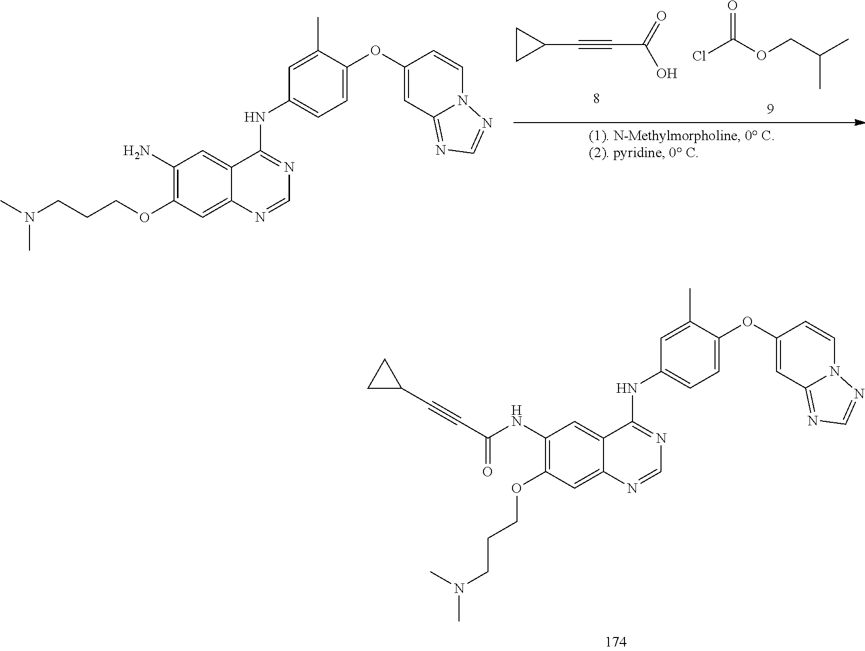

To a solution of 3-cyclopropylprop-2-ynoic acid (397.7 mg, 3.61 mmol) in THF (20.0 mL) was added 2-methylpropyl carbonochloridate (493.3 mg, 3.61 mmol) at 0° C. The mixture was stirred at 0° C. for 5 min. Then N-Methylmorpholine (730.6 mg, 7.22 mmol) was added to the mixture at 0° C. The mixture was stirred at 0° C. for another 10 min. Then a solution of 7-[3-(dimethylamino)propoxy]-N4-(3-methyl-4-[[1,2,4]triazolo[1,5-a]pyridin-7-yloxy]phenyl)quinazoline-4,6-diamine (350.0 mg, 0.72 mmol) in pyridine (2.0 mL) was added to the mixture at 0° C. The mixture was stirred at 0° C. for another 30 min. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 18% B to 44% B in 9 min; 254/220 nm) to afford 3-cyclopropyl-N-[7-[3-(dimethylamino)propoxy]-4-[(3-methyl-4-[[1,2,4]triazolo[1,5-a]pyridin-7-yloxy]phenyl)amino]quinazolin-6-yl]prop-2-ynamide (Compound 174) (11.0 mg, 2%) as a white solid. LCMS (ESI, m/z): [M+H]⁺=577.4. ¹H NMR (300 MHz, DMSO-d₆): δ 9.97 (s, 1H), 9.73 (s, 1H), 8.94 (d, J=7.5 Hz, 1H), 8.66 (s, 1H), 8.54 (s, 1H), 8.39 (s, 1H), 7.84-7.80 (m, 2H), 7.25-7.18 (m, 2H), 7.05-7.02 (m, 1H), 6.80 (d, J=2.4 Hz, 1H), 4.25-4.20 (m, 2H), 2.45-2.41 (m, 2H), 2.19-2.17 (m, 9H), 1.97-1.92 (m, 2H), 1.63-1.54 (m, 1H), 1.04-0.86 (m, 4H).

### Example S64. Synthesis of 4,4-dimethyl-N-{4-[(3-methyl-4-{[1,2,4]triazolo[1,5-a]pyridin-7-yloxy}phenyl)amino]quinazolin-6-yl}pent-2-ynamide (Compound 175)

### Step 1: Synthesis of propyl 4,4-dimethylpent-2-ynoate

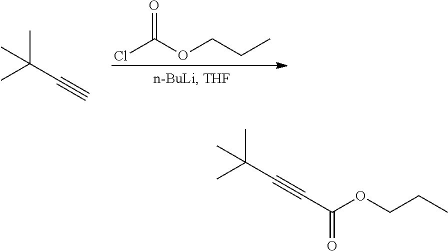

To a solution of 3,3-dimethylbut-1-yne (1.0 g, 12.17 mmol) in THF (30.0 mL) was added dropwise n-BuLi (5.8 mL, 2.5 mol/L) at −70° C. under N₂. The mixture was stirred at −70° C. for 1 h. Then propyl carbonochloridate (1.8 g, 14.61 mmol) was added dropwise to the mixture at −70° C. The mixture was stirred at −70° C. for another 2 h. After the reaction was completed, the mixture was quenched with saturated NH₄Cl solution at −70° C. The resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (85/15, v/v) to afford propyl 4,4-dimethylpent-2-ynoate (820.0 mg, 40%) as a colorless oil.

### Step 2: Synthesis of 4,4-dimethyl-N-{4-[(3-methyl-4-{[1,2,4]triazolo[1,5-a]pyridin-7-yloxy}phenyl)amino]quinazolin-6-yl}pent-2-ynamide (Compound 175)

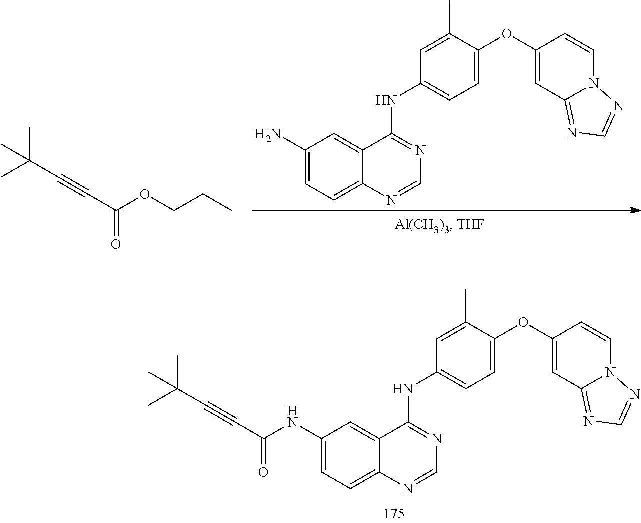 [SMILES: CCCOC(=O)C#CC(C)(C)C ; Cc1cc(Nc2ncnc3ccc(N)cc23)ccc1Oc1ccn2ncnc2c1 ; Cc1cc(Nc2ncnc3ccc(NC(=O)C#CC(C)(C)C)cc23)ccc1Oc1ccn2ncnc2c1]

To a mixture of N4-(3-methyl-4-{[1,2,4]triazolo[1,5-a]pyridin-7-yloxy}phenyl)quinazoline-4,6-diamine (300.0 mg, 0.78 mmol) and propyl 4,4-dimethylpent-2-ynoate (394.9 mg, 2.35 mmol) in THF (5.0 mL) was added dropwise trimethylaluminium (2.3 mL, 2 mol/L) at 0° C. The resulting mixture was stirred at 80° C. for 3 h. After the reaction was completed, the reaction mixture was cooled to room temperature and quenched with saturated NH₄Cl solution at room temperature. the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (92/8, v/v) and then purified by Prep-HPLC with the following conditions: (Column:) (Bridge Prep OBD C18 Column, 30×150 mm, 5 um; Mobile Phase A: Water (10 mmol/L NH₄HCO₃), Mobile Phase B: ACN; Flow rate: 60 mL/min; Gradient: 30% B to 60% B in 9 min; 254 nm) to afford 4,4-dimethyl-N-{4-[(3-methyl-4-{[1,2,4]triazolo[1,5-a]pyridin-7-yloxy}phenyl)amino]quinazolin-6-yl}pent-2-ynamide (Compound 175) (128.6 mg, 33%) as a white solid. LCMS (ESI, m/z): [M+H]⁺=492.2. ¹H NMR (300 MHz, DMSO-d₆): δ 10.84 (s, 1H), 9.88 (s, 1H), 8.95 (d, J=7.2 Hz, 1H), 8.71 (s, 1H), 8.57 (s, 1H), 8.39 (s, 1H), 7.88-7.77 (m, 4H), 7.21 (d, J=8.7 Hz, 1H), 7.06-7.02 (m, 1H), 6.81 (d, J=2.4 Hz, 1H), 2.19 (s, 3H), 1.32 (s, 9H).

### Example S65. Synthesis of 3-cyclopropyl-N-{4-[(2-fluoro-3-methyl-4-{[1,2,4]triazolo[1,5-a]pyridin-7-yloxy}phenyl)amino]quinazolin-6-yl}prop-2-ynamide (Compound 176)

### Step 1: Synthesis of N-(2-fluoro-3-methyl-4-{[1,2,4]triazolo[1,5-a]pyridin-7-yloxy}phenyl)-6-nitroquinazolin-4-amine

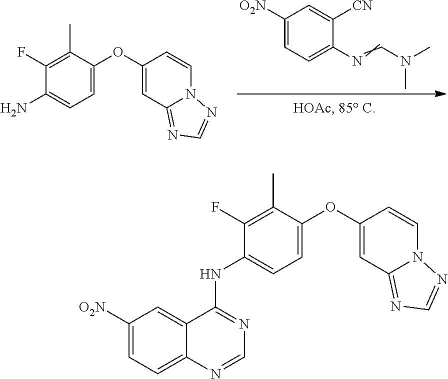 [SMILES: Cc1c(Oc2ccn3ncnc3c2)ccc(N)c1F ; CN(C)C=Nc1ccc([N+](=O)[O-])cc1C#N ; Cc1c(Oc2ccn3ncnc3c2)ccc(Nc2ncnc3ccc([N+](=O)[O-])cc23)c1F]

To a mixture of 2-fluoro-3-methyl-4-{[1,2,4]triazolo[1,5-a]pyridin-7-yloxy}aniline (500.0 mg, 1.90 mmol) in HOAc (5.0 mL) was added N′-(2-cyano-4-nitrophenyl)-N,N-dimethylformimidamide (420.0 mg, 1.90 mmol) at room temperature. The resulting mixture was stirred at 85° C. for 2 h. After the reaction was completed, the mixture was evaporated in vacuo. The residue was purified by flash column chromatography with CH₂Cl₂/MeOH (10/1, v/v) to afford N-(2-fluoro-3-methyl-4-{[1,2,4]triazolo[1,5-a]pyridin-7-yloxy}phenyl)-6-nitroquinazolin-4-amine (489.0 mg, 98%) as a yellow solid. LCMS (ESI, m/z): [M+H]⁺=432.1.

### Step 2: Synthesis of N4-(2-fluoro-3-methyl-4-{[1,2,4]triazolo[1,5-a]pyridin-7-yloxy}phenyl)quinazoline-4,6-diamine

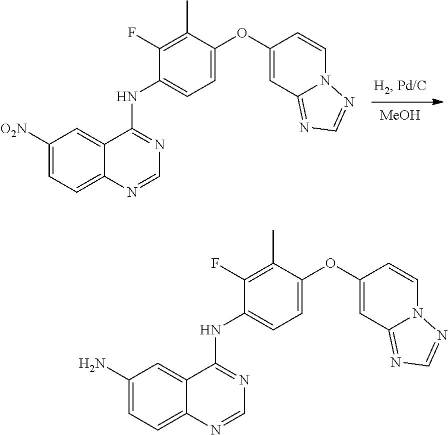 [SMILES: "MeOH" ; Cc1c(Oc2ccn3ncnc3c2)ccc(Nc2ncnc3ccc([N+](=O)[O-])cc23)c1F ; Cc1c(Oc2ccn3ncnc3c2)ccc(Nc2ncnc3ccc(N)cc23)c1F]

To a solution of N-(2-fluoro-3-methyl-4-{[1,2,4]triazolo[1,5-a]pyridin-7-yloxy}phenyl)-6-nitroquinazolin-4-amine (389.0 mg, 0.90 mmol) in MeOH (20.0 mL) was added Pd/C (177.0 mg, dry) at room temperature. The mixture was stirred at room temperature for 3 h under H₂. After the reaction was completed, the reaction mixture was filtered. The filtrate was evaporated in vacuo to afford N4-(2-fluoro-3-methyl-4-{[1,2,4]triazolo[1,5-a]pyridin-7-yloxy}phenyl)quinazoline-4,6-diamine (360.0 mg, crude) as a reddish solid. LCMS (ESI, m/z): [M+H]⁺=402.1.

### Step 3: Synthesis of 3-cyclopropyl-N-{4-[(2-fluoro-3-methyl-4-{[1,2,4]triazolo[1,5-a]pyridin-7-yloxy}phenyl)amino]quinazolin-6-yl}prop-2-ynamide (Compound 176)

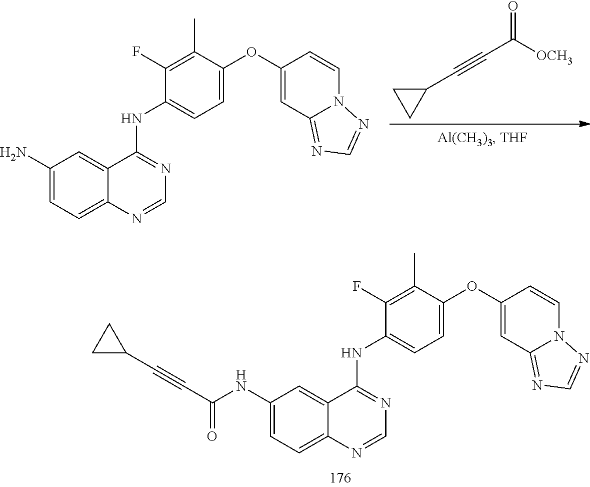

To a mixture of N4-(2-fluoro-3-methyl-4-{[1,2,4]triazolo[1,5-a]pyridin-7-yloxy}phenyl)quinazoline-4,6-diamine (200.0 mg, 0.49 mmol) and methyl 3-cyclopropylprop-2-ynoate (185.5 mg, 1.49 mmol) in THF (15.0 mL) was added dropwise AlMe₃ (1.0 mL, 2 mol/L) at 0° C. The resulting mixture was stirred at 80° C. for 3 h. After the reaction was completed, the reaction was quenched with NH₄Cl (aq) at 0° C. The resulting mixture was diluted with H₂O and extracted with DCM. The combined organic layer was washed with brine, dried over anhydrous sodium sulfate and filtered. The filtrate was concentrated under reduced pressure. The residue was purified by Prep-HPLC with the following conditions: (Column: Xselect CSH OBD Column 30×150 mm, 5 um; Mobile Phase A: Water (10 mmol/L NH₄HCO₃+0.1% NH₃·H₂O), Mobile Phase B: ACN; Flow rate: 60 mL/min; Gradient: 29% B to 40% B in 13 min, 254 nm) to afford 3-cyclopropyl-N-{4-[(2-fluoro-3-methyl-4-{[1,2,4]triazolo[1,5-a]pyridin-7-yloxy}phenyl)amino]quinazolin-6-yl}prop-2-ynamide (Compound 176) (31.1 mg, 12%) as a light yellow solid. LCMS (ESI, m/z): [M+H]⁺=494.1. ¹H NMR (400 MHz, DMSO-d₆): δ 10.88 (s, 1H), 9.84 (s, 1H), 8.99 (d, J=7.2 Hz, 1H), 8.73 (s, 1H), 8.47 (s, 1H), 8.43 (s, 1H), 7.83-7.77 (m, 2H), 7.52-7.43 (m, 1H), 7.12-7.07 (m, 2H), 6.96 (d, J=2.4 Hz, 1H), 2.16 (s, 3H), 1.65-1.58 (m, 1H), 1.04-0.96 (m, 2H), 0.92-0.84 (m, 2H).

### Example S66. Synthesis of 3-cyclopropyl-N-{4-[(2-fluoro-5-methyl-4-{[1,2,4]triazolo[1,5-a]pyridin-7-yloxy}phenyl)amino]quinazolin-6-yl}prop-2-ynamide (Compound 177)

### Step 1: Synthesis of 3-cyclopropyl-N-{4-[(2-fluoro-5-methyl-4-{[1,2,4]triazolo[1,5-a]pyridin-7-yloxy}phenyl)amino]quinazolin-6-yl}prop-2-ynamide (Compound 177)

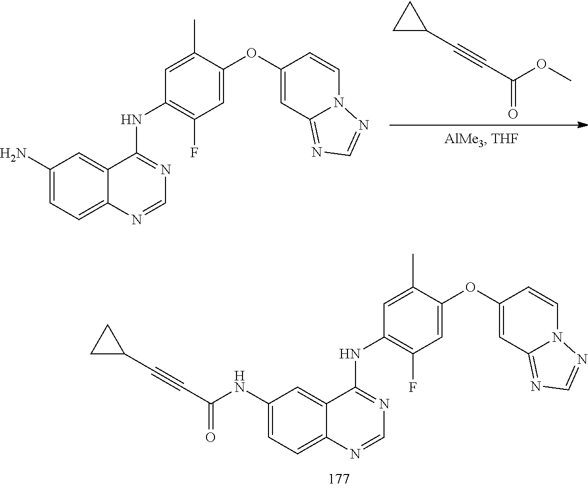

To a mixture of N4-(2-fluoro-5-methyl-4-{[1,2,4]triazolo[1,5-a]pyridin-7-yloxy}phenyl)quinazoline-4,6-diamine (200.0 mg, 0.49 mmol) and methyl 3-cyclopropylprop-2-ynoate (185.5 mg, 1.49 mmol) in THF (10.0 mL) was added dropwise AlMe₃ (1.2 mL, 2 mol/L) at 0° C. The resulting mixture was stirred at 80° C. for 3 h. After the reaction was completed, the reaction was quenched with NH₄Cl (sat.) at room temperature. The resulting mixture was diluted with H₂O and extracted with CH₂Cl₂. The combined organic layer was washed with brine, dried over anhydrous sodium sulfate and filtered. The filtrate was concentrated under reduced pressure. The residue was purified by Prep-HPLC with the following conditions: (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 25% B to 45% B in 12 min, 254 nm) to afford 3-cyclopropyl-N-{4-[(2-fluoro-5-methyl-4-{[1,2,4]triazolo[1,5-a]pyridin-7-yloxy}phenyl)amino]quinazolin-6-yl}prop-2-ynamide (Compound 177) (27.0 mg, 10%) as an off-white solid. LCMS (ESI, m/z): [M+H]⁺=494.2. ¹H NMR (400 MHz, DMSO-d₆): δ 10.86 (s, 1H), 9.85 (s, 1H), 8.97 (d, J=8.4 Hz, 1H), 8.70 (s, 1H), 8.42 (s, 2H), 7.80-7.75 (m, 2H), 7.52 (s, 1H), 7.27-7.24 (m, 1H), 7.07-7.05 (m, 1H), 6.94 (d, J=2.0 Hz, 1H), 2.17 (s, 3H), 1.64-1.58 (m, 1H), 1.00-0.87 (m, 4H).

### Example S67. Synthesis of N-(4-((4-([1,2,4]triazolo[1,5-a]pyridin-7-yloxy)−3-methylphenyl)amino)-5-fluoroquinazolin-6-yl)-3-cyclopropylpropiolamide (Compound 178)

### Step 1: Synthesis of N-(4-([1,2,4]triazolo[1,5-a]pyridin-7-yloxy)-3-methylphenyl)-6-bromo-5-fluoroquinazolin-4-amine

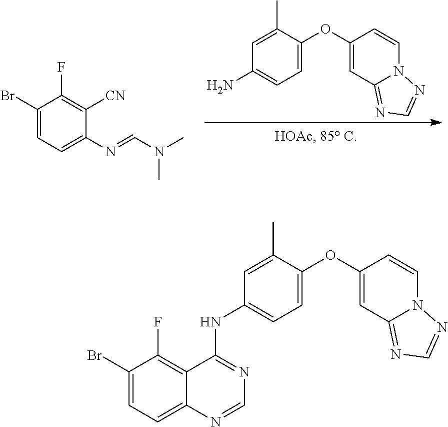 [SMILES: CN(C)/C=N/c1ccc(Br)c(F)c1C#N ; Cc1cc(N)ccc1Oc1ccn2ncnc2c1 ; "HOAc, 85° C." ; Cc1cc(Nc2ncnc3ccc(Br)c(F)c23)ccc1Oc1ccn2ncnc2c1]

A mixture of 4-([1,2,4]triazolo[1,5-a]pyridin-7-yloxy)-3-methylaniline (1.4 g, 6.66 mmol) and (E)-N′-(4-bromo-2-cyano-3-fluorophenyl)-N,N-dimethylmethanimidamide (1.2 g, 4.44 mmol) in HOAc (30.0 mL) was stirred at 85° C. for 2 h. After the reaction was completed, the resulting mixture was concentrated under reduced pressure. The residue was purified by flash column chromatography with CH₂Cl₂/MeOH (10/1, v/v) to afford N-(4-([1,2,4]triazolo[1,5-a]pyridin-7-yloxy)-3-methylphenyl)-6-bromo-5-fluoroquinazolin-4-amine (1.9 g, 92%) as a white solid. LCMS (ESI, m/z): [M+H]⁺=465.0.

### Step 2: Synthesis of tert-butyl (4-((4-([1,2,4]triazolo[1,5-a]pyridin-7-yloxy)-3-methylphenyl)amino)-5-fluoroquinazolin-6-yl)carbamate

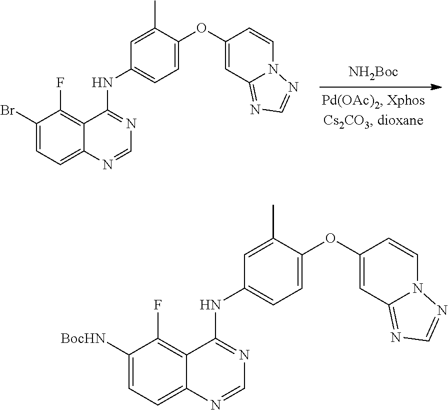 [SMILES: Cc1cc(Nc2ncnc3ccc(Br)c(F)c23)ccc1Oc1ccn2ncnc2c1 ; Cc1cc(Nc2ncnc3ccc(NC(=O)OC(C)(C)C)c(F)c23)ccc1Oc1ccn2ncnc2c1 ; "NH2Boc"]

To a solution of N-(4-([1,2,4]triazolo[1,5-a]pyridin-7-yloxy)-3-methylphenyl)-6-bromo-5-fluoroquinazolin-4-amine (1.8 g, 3.76 mmol) in dioxane (28.0 mL) was added NH₂Boc (1.3 g, 11.28 mmol), Cs₂CO₃ (3.7 g, 11.28 mmol), Pd(OAc)₂ (0.2 g, 0.75 mmol) and XPhos (0.7 g, 1.50 mmol) at room temperature. The mixture was stirred at 100° C. for 4 h under N₂. After the reaction was completed, the resulting mixture was diluted with water and extracted with EtOAc. The combined organic layer was washed with brine, dried over anhydrous sodium sulfate and filtered. The filtrate was concentrated under vacuum. The residue was purified by flash column chromatography with CH₂Cl₂/MeOH (10/1, v/v) to afford tert-butyl (4-((4-([1,2,4]triazolo[1,5-a]pyridin-7-yloxy)-3-methylphenyl)amino)-5-fluoroquinazolin-6-yl)carbamate (1.2 g, 63%) as a brown solid. LCMS (ESI, m/z): [M+H]⁺=502.2.

### Step 3: Synthesis of N4-(4-([1,2,4]triazolo[1,5-a]pyridin-7-yloxy)-3-methylphenyl)-5-fluoroquinazoline-4,6-diamine

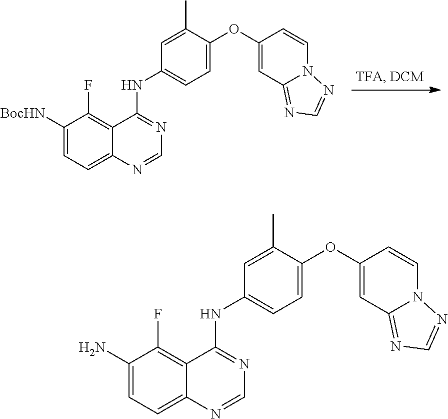

A mixture of tert-butyl N-(5-fluoro-4-[(3-methyl-4-([1,2,4]triazolo[1,5-a]pyridin-7-yloxy)phenyl)amino]quinazolin-6-yl)carbamate (1.2 g, 2.39 mmol) and TFA (6.0 mL) in DCM (6.0 mL) was stirred at room temperature for 1 h. After the reaction was completed, the resulting mixture was concentrated under reduced pressure. The residue was purified by flash column chromatography with CH₂Cl₂/MeOH (10/1, v/v/) to afford N4-(4-([1,2,4]triazolo[1,5-a]pyridin-7-yloxy)-3-methylphenyl)-5-fluoroquinazoline-4,6-diamine (400.0 mg, 41%) as a light yellow solid. LCMS (ESI, m/z): [M+H]⁺=402.1.

### Step 4: Synthesis of N-(4-((4-([1,2,4]triazolo[1,5-a]pyridin-7-yloxy)-3-methylphenyl)amino)-5-fluoroquinazolin-6-yl)-3-cyclopropylpropiolamide (Compound 178)

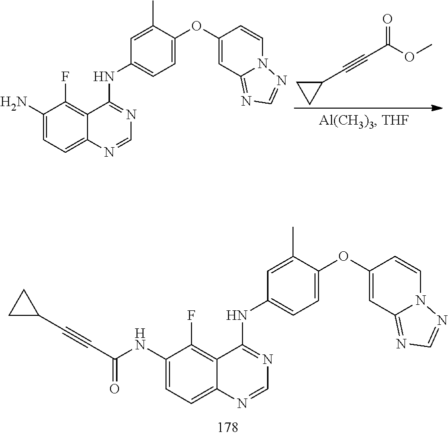

To a mixture of N4-(4-([1,2,4]triazolo[1,5-a]pyridin-7-yloxy)-3-methylphenyl)-5-fluoroquinazoline-4,6-diamine (400.0 mg, 1.00 mmol) and methyl 3-cyclopropylprop-2-ynoate (371.1 mg, 3.00 mmol) in THF (15.0 mL) was added dropwise AlMe₃ (0.5 mL, 2 mol/L) at 0° C. The resulting mixture was stirred at 85° C. for 3 h. After the reaction was completed, the reaction was quenched with sat. NH₄Cl (aq.) and extracted with CH₂Cl₂. The combined organic layer was washed with brine, dried over anhydrous Na₂SO₄ and filtered. The filtrate was concentrated under reduced pressure. The residue was purified by flash column chromatography with CH₂Cl₂/MeOH (10/1, v/v) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 33% B to 45% B in 9 min; 254/220 nm) to afford N-(4-((4-([1,2,4]triazolo[1,5-a]pyridin-7-yloxy)-3-methylphenyl)amino)-5-fluoroquinazolin-6-yl)-3-cyclopropylpropiolamide (Compound 178) (67.3 mg, 13%) as a yellow solid. LCMS (ESI, m/z): [M+H]⁺=494.3. ¹H NMR (300 MHz, DMSO-d₆): δ 10.62 (s, 1H), 9.17 (s, 1H), 8.93 (d, J=7.5 Hz, 1H), 8.55 (s, 1H), 8.37 (s, 1H), 8.01-7.96 (m, 1H), 7.73-7.60 (m, 3H), 7.20 (d, J=8.7 Hz, 1H), 7.03-7.00 (m, 1H), 6.78 (d, J=2.4 Hz, 1H), 2.17 (s, 3H), 1.64-1.50 (m, 1H), 0.98-0.83 (m, 4H).

### Example S68. Synthesis of N-(4-((4-([1,2,4]triazolo[1,5-a]pyridin-7-yloxy)-2-fluoro-3-methylphenyl)amino)-7-(2-(dimethylamino)ethoxy)quinazolin-6-yl)-3-cyclopropylpropiolamide (Compound 179)

### Step 1: Synthesis of N-(4-([1,2,4]triazolo[1,5-a]pyridin-7-yloxy)-2-fluoro-3-methylphenyl)-7-(2-(dimethylamino)ethoxy)-6-nitroquinazolin-4-amine

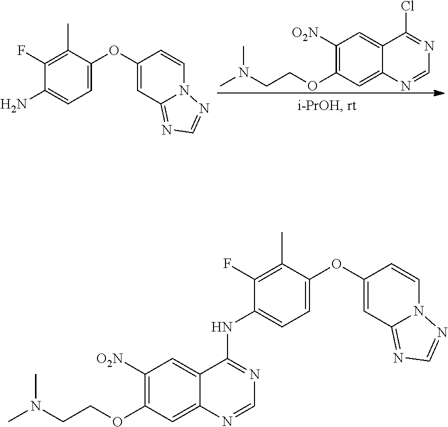

To a solution of 4-([1,2,4]triazolo[1,5-a]pyridin-7-yloxy)-2-fluoro-3-methylaniline (500.0 mg, 1.94 mmol) in i-PrOH (20.0 mL) was added 2-((4-chloro-6-nitroquinazolin-7-yl)oxy)-N,N-dimethylethan-1-amine (574.5 mg, crude) at room temperature. The resulting mixture was stirred at room temperature for 3 h. After the reaction was completed, the reaction mixture was concentrated under vacuum. The residue was purified by flash column chromatography with CH₂Cl₂/CH₃OH (10/1, v/v) to afford N-(4-([1,2,4]triazolo[1,5-a]pyridin-7-yloxy)-2-fluoro-3-methylphenyl)-7-(2-(dimethylamino)ethoxy)-6-nitroquinazolin-4-amine (800.0 mg, 79%) as a yellow solid. LCMS (ESI, m/z): [M+H]⁺=519.2.

### Step 2: Synthesis of N⁴-(4-([1,2,4]triazolo[1,5-a]pyridin-7-yloxy)-2-fluoro-3-methylphenyl)-7-(2-(dimethylamino)ethoxy)quinazoline-4,6-diamine

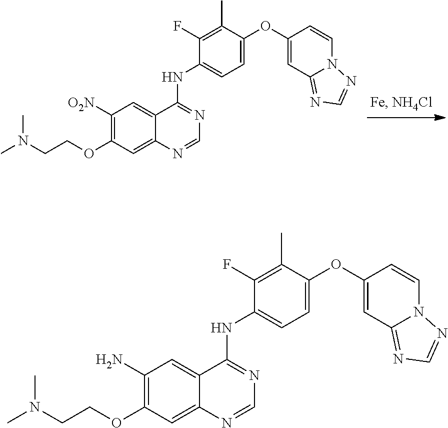 [SMILES: Cc1c(Oc2ccn3ncnc3c2)ccc(Nc2ncnc3cc(OCCN(C)C)c([N+](=O)[O-])cc23)c1F ; Cc1c(Oc2ccn3ncnc3c2)ccc(Nc2ncnc3cc(OCCN(C)C)c(N)cc23)c1F]

To a solution of N-(4-([1,2,4]triazolo[1,5-a]pyridin-7-yloxy)-2-fluoro-3-methylphenyl)-7-(2-(dimethylamino)ethoxy)-6-nitroquinazolin-4-amine (800.0 mg, 1.54 mmol) in EtOH/H₂O (16.0/4.0 mL) was added NH₄Cl (330.1 mg, 6.17 mmol) at room temperature. Then Fe (258.5 mg, 4.63 mmol) was added to the mixture at 80° C. The resulting mixture was stirred at 80° C. for 4 h. After the reaction was completed, the resulting mixture was filtered. The filtrate was concentrated under reduced pressure. The residue was purified by flash column chromatography with CH₂Cl₂/CH₃OH (5/1, v/v) to afford N⁴-(4-([1,2,4]triazolo[1,5-a]pyridin-7-yloxy)-2-fluoro-3-methylphenyl)-7-(2-(dimethylamino)ethoxy)quinazoline-4,6-diamine (400.0 mg, 53%) as a yellow solid. LCMS (ESI, m/z): [M+H]⁺=489.2.

### Step 3: Synthesis of N-(4-((4-([1,2,4]triazolo[1,5-a]pyridin-7-yloxy)-2-fluoro-3-methylphenyl)amino)-7-(2-(dimethylamino)ethoxy)quinazolin-6-yl)-3-cyclopropylpropiolamide (Compound 179)

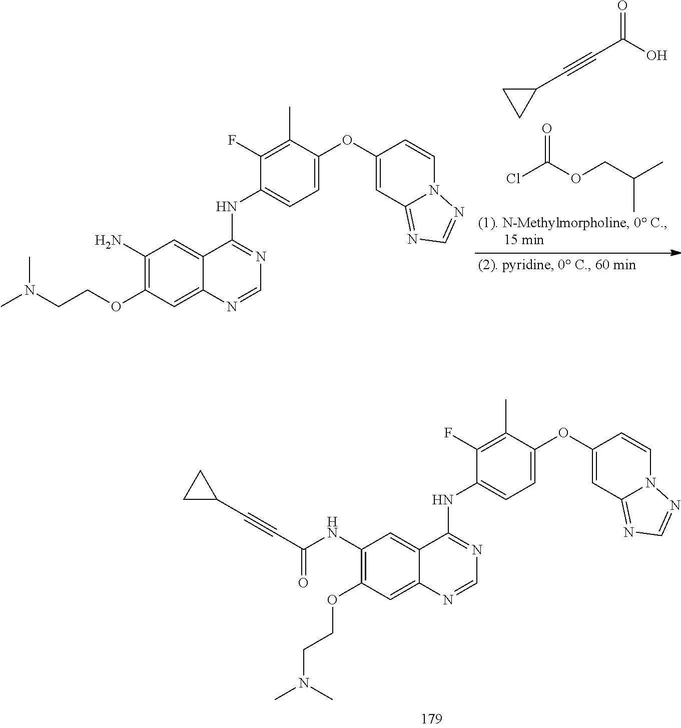

To a solution of cyclopropylpropiolic acid (202.9 mg, 1.84 mmol) in THF (10.0 mL) was added 2-methylpropyl carbonochloridate (251.6 mg, 1.84 mmol) at 0° C. The mixture was stirred at 0° C. for 5 min. Then N-Methylmorpholine (372.7 mg, 3.68 mmol) was added to the mixture at 0° C. The mixture was stirred at 0° C. for another 10 min. Then a solution of N⁴-(4-([1,2,4]triazolo[1,5-a]pyridin-7-yloxy)-2-fluoro-3-methylphenyl)-7-(2-(dimethylamino)ethoxy)quinazoline-4,6-diamine (180.0 mg, 0.37 mmol) in pyridine (1.0 mL) was added to the mixture at 0° C. The mixture was stirred at 0° C. for another 30 min. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (10/1, v/v) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 33% to 47% in 9 min; 254 nm) to afford N-(4-((4-([1,2,4]triazolo[1,5-a]pyridin-7-yloxy)-2-fluoro-3-methylphenyl)amino)-7-(2-(dimethylamino)ethoxy)quinazolin-6-yl)-3-cyclopropylpropiolamide (Compound 179) (42.7 mg, 19%) as a yellow solid. LCMS (ESI, m/z): [M+H]⁺=581.4. ¹H NMR (400 MHz, DMSO-d₆): δ 8.98 (d, J=7.2 Hz, 1H), 8.69 (s, 1H), 8.43 (s, 2H), 7.45-7.35 (m, 2H), 7.10-6.69 (m, 3H), 4.31-4.29 (m, 2H), 2.73-2.70 (m, 2H), 2.33-2.29 (m, 6H), 2.16 (s, 3H), 1.65-1.57 (m, 1H), 1.02-0.88 (m, 4H).

### Example S69. Synthesis of N-(4-((4-([1,2,4]triazolo[1,5-a]pyridin-7-yloxy)-2-fluoro-3-methylphenyl)amino)-7-(2-(dimethylamino)ethoxy)quinazolin-6-yl)but-2-ynamide (Compound 180)

### Step 1: Synthesis of N-(4-((4-([1,2,4]triazolo[1,5-a]pyridin-7-yloxy)-2-fluoro-3-methylphenyl)amino)-7-(2-(dimethylamino)ethoxy)quinazolin-6-yl)but-2-ynamide (Compound 180)

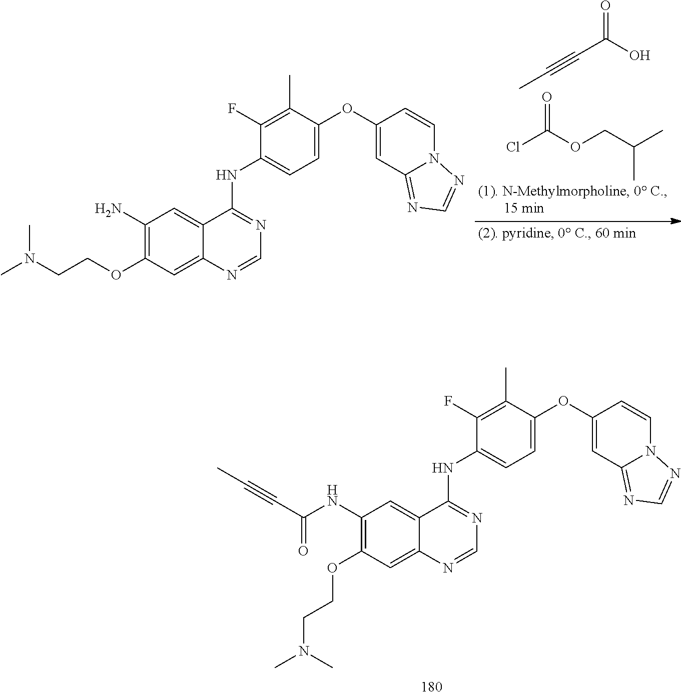

To a solution of but-2-ynoic acid (154.9 mg, 1.84 mmol) in THF (20.0 mL) was added isobutyl carbonochloridate (251.6 mg, 1.84 mmol) at 0° C. The mixture was stirred at 0° C. for 5 min. Then N-Methylmorpholine (372.7 mg, 3.68 mmol) was added to the mixture at 0° C. The mixture was stirred at 0° C. for another 10 min. Then a solution of N⁴-(4-([1,2,4]triazolo[1,5-a]pyridin-7-yloxy)-2-fluoro-3-methylphenyl)-7-(2-(dimethylamino)ethoxy)quinazoline-4,6-diamine (180.00 mg, 0.37 mmol) in Pyridine (1.0 mL) was added to the mixture at 0° C. The mixture was stirred at 0° C. for another 30 min. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (10/1, v/v) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 27% to 37% in 10 min; 254 nm) to afford N-(4-((4-([1,2,4]triazolo[1,5-a]pyridin-7-yloxy)-2-fluoro-3-methylphenyl)amino)-7-(2-(dimethylamino)ethoxy)quinazolin-6-yl)but-2-ynamide (Compound 180) (51.7 mg, 25%) as a white solid. LCMS (ESI, m/z): [M+H]⁺=555.3. ¹H NMR (400 MHz, DMSO-d₆): δ 10.25 (s, 1H), 9.75 (s, 1H), 8.98 (d, J=7.6 Hz, 1H), 8.71 (s, 1H), 8.44-8.42 (m, 2H), 7.48-7.44 (m, 1H), 7.36 (s, 1H), 7.10-7.07 (m, 2H), 6.96 (d, J=2.4 Hz, 1H), 4.32-4.29 (m, 2H), 2.73-2.65 (m, 2H), 2.33 (s, 6H), 2.16-2.08 (m, 6H).

### Example S70. Synthesis of N-(4-(4-([1,2,4]triazolo[1,5-a]pyridin-7-yloxy)-2-fluoro-5-methylphenylamino)-7-(2-(dimethylamino)ethoxy)quinazolin-6-yl)-3-cyclopropylpropiolamide formate (Compound 181)

### Step 1: Synthesis of N-(4-(4-([1,2,4]triazolo[1,5-a]pyridin-7-yloxy)-2-fluoro-5-methylphenylamino)-7-(2-(dimethylamino)ethoxy)quinazolin-6-yl)-3-cyclopropylpropiolamide formate (Compound 181)

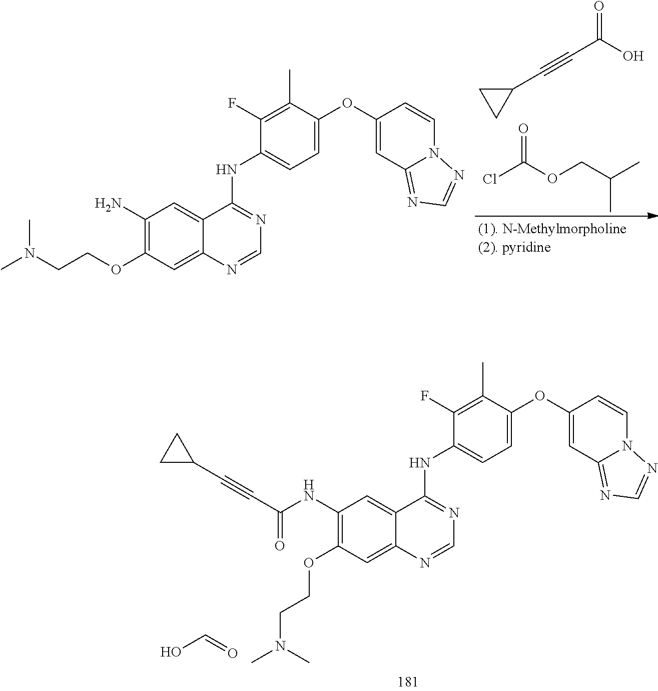

To a solution of 3-cyclopropylprop-2-ynoic acid (436.8 mg, 3.97 mmol) in THF (20.0 mL) was added 2-methylpropyl carbonochloridate (714.0 mg, 5.20 mmol) at 0° C. under N2. The resulting mixture was stirred at 0 C for 5 min. Then N-Methylmorpholine (729.5 mg, 7.22 mmol) was added to the mixture at 0° C. The resulting mixture was stirred for at 0° C. for another 10 min. Then a solution of 7-[2-(dimethylamino)ethoxy]-N4-(2-fluoro-5-methyl-4-{[1,2,4]triazolo[1,5-a]pyridin-7-yloxy}phenyl)quinazoline-4,6-diamine (500.0 mg, 0.90 mmol) in pyridine (2.0 mL) was added dropwise to the mixture at 0 C. The resulting mixture was stirred at 0 C for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (20:1 v/v) and then purified by Prep-HPLC with the following conditions: (Column: Xselect CSH OBD Column 30×150 mm, 5 um; Mobile Phase A: Water (0.1% FA), Mobile Phase B: ACN; Flow rate: 60 mL/min; Gradient: 10% B to 35% B in 8 min, 254 nm) to afford N-(4-(4-([1,2,4]triazolo[1,5-a]pyridin-7-yloxy)-2-fluoro-5-methylphenylamino)-7-(2-(dimethylamino)ethoxy)quinazolin-6-yl)-3-cyclopropylpropiolamide formate (Compound 181) (33.9 mg, 6%) as a white solid. LCMS (ESI, m/z): [M+H]⁺=581.4. ¹H NMR (400 MHz, DMSO-d₆): δ 10.28 (s, 1H), 9.76 (s, 1H), 8.98 (d, J=7.6 Hz, 1H), 8.71 (s, 1H), 8.44 (d, J=4.0 Hz, 2H), 8.22 (s, 1H), 7.53 (d, J=8.4 Hz, 1H), 7.35 (s, 1H), 7.29-7.26 (m, 1H), 7.09-7.06 (m, 1H), 6.95 (d, J=2.4 Hz, 1H), 4.35-4.30 (m, 2H), 2.88-2.78 (m, 2H), 2.34 (s, 6H), 2.19 (s, 3H), 1.65-1.55 (m, 1H), 1.00-0.94 (m, 2H), 0.86-0.82 (m, 2H).

### Example S71. Synthesis of N-{7-[2-(dimethylamino)ethoxy]-4-[(2-fluoro-5-methyl-4-{[1,2,4]triazolo[1,5-a]pyridin-7-yloxy}phenyl)amino]quinazolin-6-yl}but-2-ynamide (Compound 182)

### Step 1: Synthesis of 7-[2-(dimethylamino)ethoxy]-N-(2-fluoro-5-methyl-4-{[1,2,4]triazolo[1,5-a]pyridin-7-yloxy}phenyl)-6-nitroquinazolin-4-amine

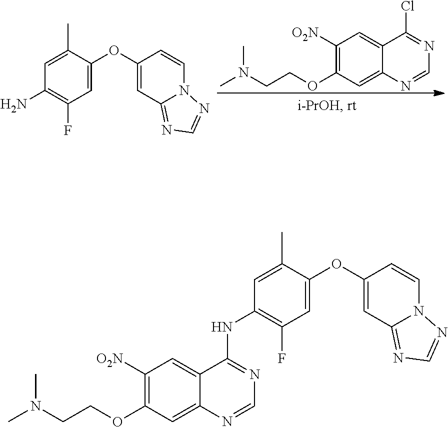

To a solution of [2-[(4-chloro-6-nitroquinazolin-7-yl)oxy]ethyl]dimethylamine (367.0 mg, crude) in i-PrOH (5.0 mL) was added 4-([1,2,4]triazolo[1,5-a]pyridin-7-yloxy)-2-fluoro-5-methylaniline (320.0 mg, 1.31 mmol) at room temperature. The resulting mixture was stirred at room temperature for 1 h. After the reaction was completed, the mixture was concentrated under vacuum. The residue was purified by flash column chromatography with CH₂Cl₂/MeOH (10/1, v/v) to afford 7-[2-(dimethylamino)ethoxy]-N-(2-fluoro-5-methyl-4-{[1,2,4]triazolo[1,5-a]pyridin-7-yloxy}phenyl)-6-nitroquinazolin-4-amine (134.0 mg, 20%) as a yellow solid. LCMS (ESI, m/z): [M+H]⁺=519.2.

### Step 2: Synthesis of 7-[2-(dimethylamino)ethoxy]-N4-(2-fluoro-5-methyl-4-{[1,2,4]triazolo[1,5-a]pyridin-7-yloxy}phenyl)quinazoline-4,6-diamine

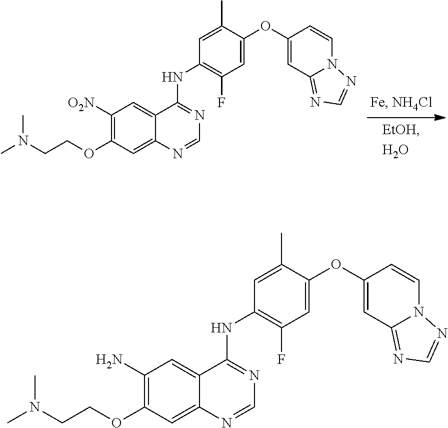 [SMILES: Cc1cc(Nc2ncnc3cc(OCCN(C)C)c([N+](=O)[O-])cc23)c(F)cc1Oc1ccn2ncnc2c1 ; Cc1cc(Nc2ncnc3cc(OCCN(C)C)c(N)cc23)c(F)cc1Oc1ccn2ncnc2c1]

To a mixture of 7-[2-(dimethylamino)ethoxy]-N-(2-fluoro-5-methyl-4-{[1,2,4]triazolo[1,5-a]pyridin-7-yloxy}phenyl)-6-nitroquinazolin-4-amine (350.0 mg, 0.68 mmol) and NH₄Cl (108.3 mg, 2.03 mmol) in EtOH (8.0 mL)/H₂O (1.6 mL) was added Fe (113.1 mg, 2.03 mmol) at 80° C. The resulting mixture was stirred at 80° C. for 3 h. After the reaction was completed, the resulting mixture was filtered. The filtrate was concentrated under reduced pressure. The mixture was purified by flash column chromatography with CH₂Cl₂/MeOH (5/1, v/v) to afford 7-[2-(dimethylamino)ethoxy]-N4-(2-fluoro-5-methyl-4-{[1,2,4]triazolo[1,5-a]pyridin-7-yloxy}phenyl)quinazoline-4,6-diamine (300.0 mg, 90%) as a yellow solid. LCMS (ESI, m/z): [M+H]⁺=489.2.

### Step 3: Synthesis of N-{7-[2-(dimethylamino)ethoxy]-4-[(2-fluoro-5-methyl-4-{[1,2,4]triazolo[1,5-a]pyridin-7-yloxy}phenyl)amino]quinazolin-6-yl}but-2-ynamide (Compound 182)

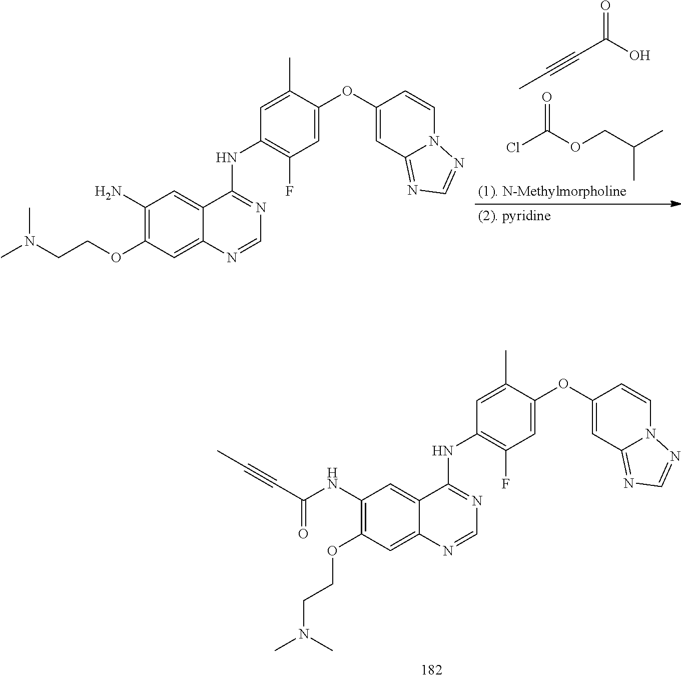

To a solution of 2-butynoic acid (162.1 mg, 1.93 mmol) in THF (14.0 mL) was added 2-methylpropyl carbonochloridate (353.0 mg, 2.58 mmol) at 0° C. under N₂. The resulting mixture was stirred at 0° C. for 5 min. Then N-Methylmorpholine (354.5 mg, 3.50 mmol) was added to the mixture at 0° C. under N₂. The mixture was stirred at 0° C. for another 10 min. Then a solution of 7-[2-(dimethylamino)ethoxy]-N4-(2-fluoro-5-methyl-4-{[1,2,4]triazolo[1,5-a]pyridin-7-yloxy}phenyl)quinazoline-4,6-diamine (214.0 mg, 0.44 mmol) in pyridine (7.0 mL) was added dropwise to the mixture at 0° C. The mixture was stirred at 0° C. for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions: (Column: YMC-Actus Triart C18 ExRS, 30×150 mm, 5 um; Mobile Phase A: Water (10 mmol/L NH₄HCO₃), Mobile Phase B: ACN; Flow rate: 60 mL/min; Gradient: 20% B to 38% B in 10 min, 254 nm) to afford N-{7-[2-(dimethylamino)ethoxy]-4-[(2-fluoro-5-methyl-4-{[1,2,4]triazolo[1,5-a]pyridin-7-yloxy}phenyl)amino]quinazolin-6-yl}but-2-ynamide (Compound 182) (11.4 mg, 4%) as a white solid. LCMS (ESI, m/z): [M+H]⁺=555.2. ¹H NMR (400 MHz, DMSO-d₆): δ 10.26 (s, 1H), 9.77 (s, 1H), 8.98 (d, J=7.6 Hz, 1H), 8.71 (s, 1H), 8.44-8.43 (m, 2H), 7.53 (d, J=8.0 Hz, 1H), 7.36 (s, 1H), 7.27 (d, J=10.8 Hz, 1H), 7.08-7.06 (m, 1H), 6.95 (d, J=2.0 Hz, 1H), 4.31-4.29 (m, 2H), 2.73-2.67 (m, 2H), 2.28 (s, 6H), 2.18 (s, 3H), 2.09 (s, 3H).

### Example S72. Synthesis of 5-((4-((4-([1,2,4]triazolo[1,5-a]pyridin-7-yloxy)−3-methylphenyl)amino)quinazolin-6-yl)amino)-5-oxopent-3-yn-1-yl phosphate sodium salt (Compound 183)

### Step 1: Synthesis of propyl 5-((tetrahydro-2H-pyran-2-yl)oxy)pent-2-ynoate

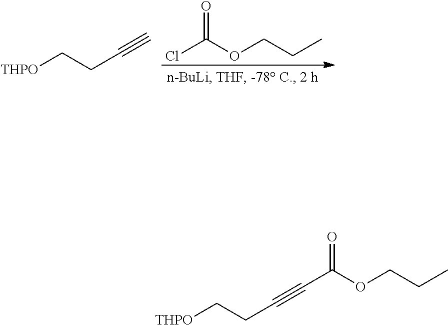

To a solution of 2-(but-3-yn-1-yloxy)tetrahydro-2H-pyran (20.0 g, 129.99 mmol, 1.00 equiv) in THF (200.0 mL) at −70° C. was added dropwise n-BuLi (2.5 M in hexanes, 57.0 mL, 142.98 mmol, 1.10 equiv) under N₂. The mixture was stirred at this temperature for 1 h and to this was added dropwise propyl carbonochloridate (26.9 g, 220.77 mmol, 1.70 equiv). The mixture was stirred at −70° C. for another 2 h and then quenched with NH₄Cl solution. The resulting mixture was diluted with H₂O and extracted with EtOAc 3 times. The combined organic layers were washed with brine, dried over Na₂SO₄ and concentrated under vacuum. The residue was purified by flash column chromatography on silica gel (0-20% EtOAc in petroleum ether) to afford propyl 5-((tetrahydro-2H-pyran-2-yl)oxy)pent-2-ynoate (27.4 g, 87%) as colorless oil. ¹H NMR (400 MHz, DMSO-d₆) δ 4.64 (t, J=3.6 Hz, 1H), 4.07 (t, J=6.4 Hz, 2H), 3.83-3.68 (m, 2H), 3.59-3.40 (m, 2H), 2.69 (t, J=6.4 Hz, 2H), 1.79-1.54 (m, 4H), 1.54-1.47 (m, 2H), 1.47-1.39 (m, 2H), 0.89 (t, J=7.6 Hz, 3H)

### Step 2: Synthesis of N-(4-((4-([1,2,4]triazolo[1,5-a]pyridin-7-yloxy)-3-methylphenyl)amino)quinazolin-6-yl)-5-((tetrahydro-2H-pyran-2-yl)oxy)pent-2-ynamide

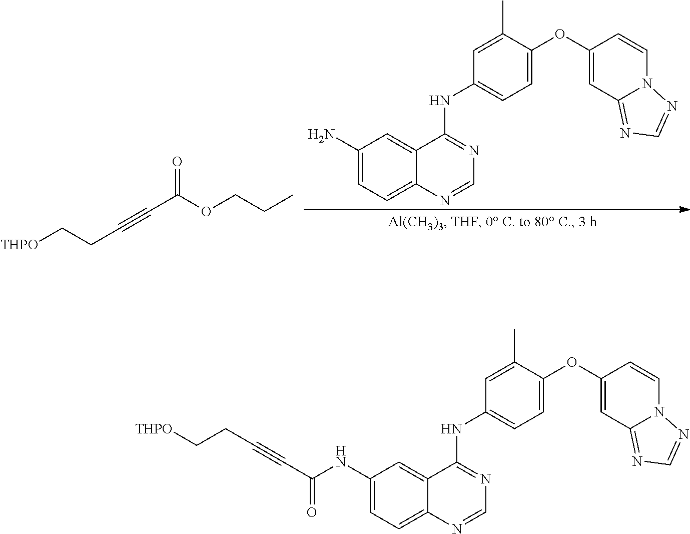

To a stirred solution of N4-(4-([1,2,4]triazolo[1,5-a]pyridin-7-yloxy)-3-methylphenyl)quinazoline-4,6-diamine (7.5 g, 19.58 mmol, 1.00 equiv) and propyl 5-((tetrahydro-2H-pyran-2-yl)oxy)pent-2-ynoate (14.1 g, 58.75 mmol, 3.00 equiv) in THF (150.0 mL) at 0° C. was added dropwise Al(CH₃)₃ (1 M in heptane, 98.0 mL, 97.91 mmol, 5.00 equiv). The resulting mixture was stirred at 80° C. for 3 h and then quenched at 0° C. with NH₄Cl solution and extracted with EtOAc 3 times. The combined organic layers was washed with brine, dried over Na₂SO₄ and concentrated under vacuum. The residue was purified by flash chromatography on silica gel (0-10% MeOH in DCM) to afford N-(4-((4-([1,2,4]triazolo[1,5-a]pyridin-7-yloxy)-3-methylphenyl)amino)quinazolin-6-yl)-5-((tetrahydro-2H-pyran-2-yl)oxy)pent-2-ynamide (8.7 g, 79%) as a yellow solid. ¹H NMR (400 MHz, DMSO-d₆) δ 10.96 (brs, 1H), 9.89 (s, 1H), 8.94 (d, J=7.6 Hz, 1H), 8.79-8.74 (m, 1H), 8.56 (s, 1H), 8.38 (s, 1H), 7.86-7.74 (m, 4H), 7.20 (d, J=8.8 Hz, 1H), 7.03 (dd, J=7.6, 2.6 Hz, 1H), 6.81 (d, J=2.4 Hz, 1H), 4.72-4.63 (m, 1H), 3.86-3.75 (m, 2H), 3.67-3.57 (m, 1H), 3.52-3.42 (m, 1H), 2.75 (t, J=6.5 Hz, 2H), 2.19 (s, 3H), 1.81-1.59 (m, 2H), 1.55-1.40 (m, 4H). MS (ESI, m/z): 564 (M+H)⁺.

### Step 3: Synthesis of N-(4-((4-([1,2,4]triazolo[1,5-a]pyridin-7-yloxy)-3-methylphenyl)amino)quinazolin-6-yl)-5-hydroxypent-2-ynamide

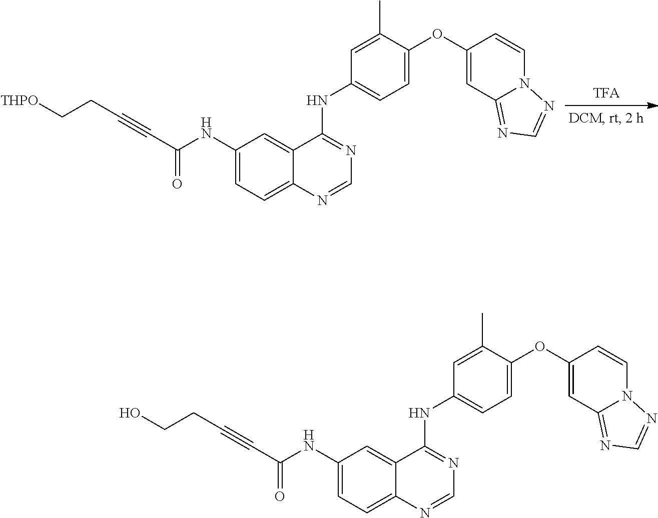

To a stirred solution of N-(4-((4-([1,2,4]triazolo[1,5-a]pyridin-7-yloxy)-3-methylphenyl)amino)quinazolin-6-yl)-5-((tetrahydro-2H-pyran-2-yl)oxy)pent-2-ynamide (8.7 g, 15.45 mmol, 1.00 equiv) in DCM (100.0 mL) at ambient temperature was added dropwise TFA (15.0 mL). The mixture was stirred at this temperature for 2 h and then neutralized with NaHCO₃ solution. The precipitated solid was collected by filtration and washed with H₂O. The product was further purified by trituration with acetonitrile to afford N-(4-((4-([1,2,4]triazolo[1,5-a]pyridin-7-yloxy)-3-methylphenyl)amino)quinazolin-6-yl)-5-hydroxypent-2-ynamide (6.0 g, 81%) as a yellow solid. The submitted sample was further purified by Prep-HPLC (NH₄HCO₃/NH₃·H₂O system) from 100 mg to afford the product (54.7 mg) as yellow solid. ¹H NMR (400 MHz, DMSO-d₆) δ 10.92 (s, 1H), 9.88 (s, 1H), 8.97-8.91 (m, 1H), 8.77-8.72 (m, 1H), 8.56 (s, 1H), 8.39 (s, 1H), 7.86-7.75 (m, 4H), 7.21 (d, J=8.8 Hz, 1H), 7.04 (dd, J=7.6, 2.6 Hz, 1H), 6.81 (d, J=2.8 Hz, 1H), 5.02 (t, J=5.6 Hz, 1H), 3.64 (q, J=6.4 Hz, 2H), 2.60 (t, J=6.4 Hz, 2H), 2.20 (s, 3H). MS (ESI, m/z): 480 (M+H)⁺.

### Step 4: Synthesis of 5-((4-((4-([1,2,4]triazolo[1,5-a]pyridin-7-yloxy)-3-methylphenyl)amino)quinazolin-6-yl)amino)-5-oxopent-3-yn-1-yl di-tert-butyl phosphate

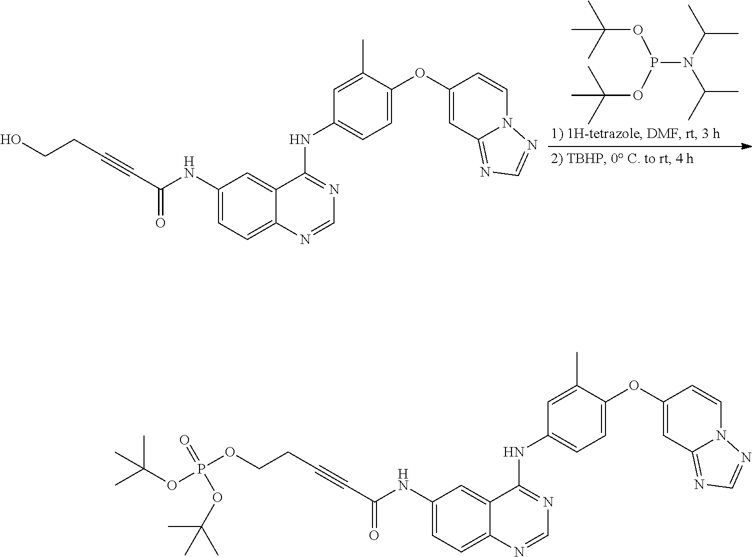 [SMILES: Cc1cc(Nc2ncnc3ccc(NC(=O)C#CCCO)cc23)ccc1Oc1ccn2ncnc2c1 ; CC(C)N(C(C)C)P(OC(C)(C)C)OC(C)(C)C ; Cc1cc(Nc2ncnc3ccc(NC(=O)C#CCCOP(=O)(OC(C)(C)C)OC(C)(C)C)cc23)ccc1Oc1ccn2ncnc2c1]

N-(4-((4-([1,2,4]triazolo[1,5-a]pyridin-7-yloxy)-3-methylphenyl)amino)quinazolin-6-yl)-5-hydroxypent-2-ynamide (6.0 g, 12.52 mmol, 1.00 equiv) was co-evaporated with anhydrous acetonitrile for 3 times and then dissolved in acetonitrile/DMF (50.0 ml/50.0 ml) under N₂ atmosphere. To this at 0° C. was added dropwise di-tert-butyl diisopropylphosphoramidite (17.3 g, 62.62 mmol, 5.00 equiv). The resulting mixture was stirred at ambient temperature for 3 h and to this at 0° C. was added dropwise TBHP (70% in H₂O, 24.2 g, 187.89 mmol, 15.00 equiv). The mixture was stirred at ambient temperature for another 1 h and then diluted with EtOAc. The organic solution was washed with NaHCO₃ solution and H₂O, dried over Na₂SO₄ and concentrated under vacuum. The residue was purified by flash chromatography on silica gel (0-10% MeOH in DCM) to afford 5-((4-((4-([1,2,4]triazolo[1,5-a]pyridin-7-yloxy)-3-methylphenyl)amino)quinazolin-6-yl)amino)-5-oxopent-3-yn-1-yl di-tert-butyl phosphate (5.1 g, 60%) as a yellow solid. ¹H NMR (400 MHz, DMSO-d₆) δ 11.04 (brs, 1H), 9.95 (s, 1H), 8.94 (d, J=7.2 Hz, 1H), 8.78 (s, 1H), 8.56 (s, 1H), 8.39 (s, 1H), 7.87-7.75 (m, 4H), 7.21 (d, J=8.8 Hz, 1H), 7.04 (dd, J=7.6, 2.6 Hz, 1H), 6.81 (d, J=2.8 Hz, 1H), 4.05 (q, J=6.4 Hz, 2H), 2.88-2.80 (m, 2H), 2.20 (s, 3H), 1.43 (d, J=12.9 Hz, 18H). MS (ESI, m/z): 672 (M+H)

### Step 5: Synthesis of 5-((4-((4-([1,2,4]triazolo[1,5-a]pyridin-7-yloxy)-3-methylphenyl)amino)quinazolin-6-yl)amino)-5-oxopent-3-yn-1-yl phosphate sodium salt (Compound 183)

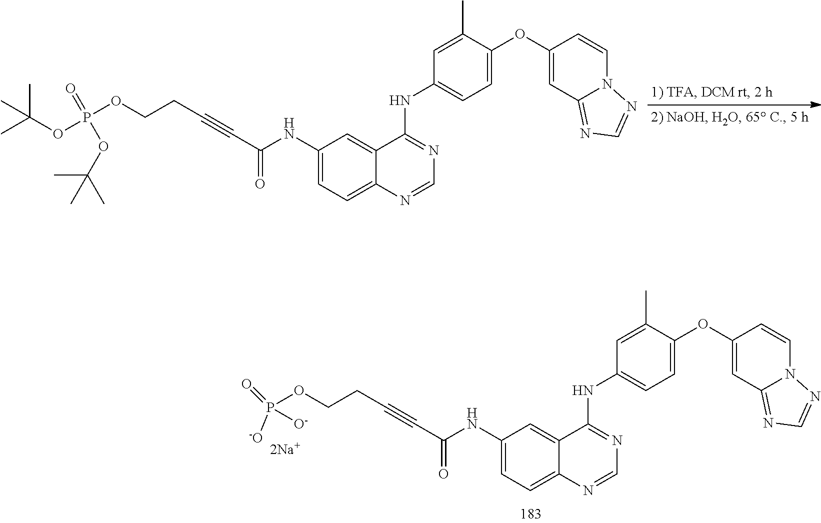

To a stirred solution of 5-((4-((4-([1,2,4]triazolo[1,5-a]pyridin-7-yloxy)-3-methylphenyl)amino)quinazolin-6-yl)amino)-5-oxopent-3-yn-1-yl di-tert-butyl phosphate (2.0 g, 2.98 mmol, 1.00 equiv) in DCM (40.0 mL) at ambient temperature was added dropwise TFA (20.0 mL). The mixture was stirred at this temperature for 2 h and then neutralized with NaOH solution (1M). The resulting mixture was washed with EtOAc 3 times. To the aqueous layer at 0° C. was added NaOH (476.8 mg, 11.92 mmol, 4.00 equiv). The resulting mixture was stirred at 65° C. for 5 h and then allowed to cool down to ambient temperature. The mixture was directly purified by reverse phase flash chromatography on C₁₈ gel (0-20% MeOH in H₂O) to afford 5-((4-((4-([1,2,4]triazolo[1,5-a]pyridin-7-yloxy)-3-methylphenyl)amino)quinazolin-6-yl)amino)-5-oxopent-3-yn-1-yl phosphate sodium salt (Compound 183) (1.18 g, 65%) as yellow solid. ¹H NMR (400 MHz, Deuterium Oxide) 6 8.18 (d, J=7.6 Hz, 1H), 8.05 (d, J=1.2 Hz, 1H), 7.97 (d, J=1.2 Hz, 1H), 7.87 (s, 1H), 7.42-7.32 (m, 2H), 7.16 (s, 1H), 7.04 (d, J=9.2 Hz, 1H), 6.87 (d, J=8.0 Hz, 1H), 6.63 (d, J=7.6 Hz, 1H), 6.44 (s, 1H), 3.89 (q, J=6.4 Hz, 2H), 2.70 (t, J=6.4 Hz, 2H), 1.91 (s, 3H). MS (ESI, m/z): 560 (M+H)⁺.

### Example S73. Synthesis of N-[4-[(3-methyl-4-[[6-(trifluoromethyl)pyridin-3-yl]oxy]phenyl)amino]quinazolin-6-yl]but-2-ynamide (Compound 184)

### Step 1: Synthesis of 5-(2-methyl-4-nitrophenoxy)-2-(trifluoromethyl)pyridine

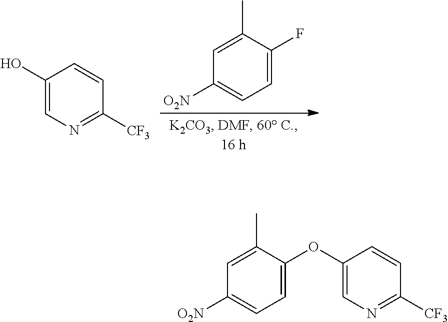 [SMILES: Oc1ccc(C(F)(F)F)nc1 ; Cc1cc([N+](=O)[O-])ccc1F ; Cc1cc([N+](=O)[O-])ccc1Oc1ccc(C(F)(F)F)nc1]

To a mixture of 6-(trifluoromethyl)pyridin-3-ol (4.5 g, 27.59 mmol) in DMF (40.0 mL) was added 1-fluoro-2-methyl-4-nitrobenzene (4.2 g, 27.59 mmol) and K₂CO₃ (11.4 g, 82.77 mmol) at room temperature. The resulting mixture was stirred at 60° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/EtOAc (2/1, v/v) to afford 5-(2-methyl-4-nitrophenoxy)-2-(trifluoromethyl)pyridine (7.0 g, 97%) as a light yellow oil. LCMS (ESI, m/z): [M+H]⁺=299.1.

### Step 2: Synthesis of 3-methyl-4-[[6-(trifluoromethyl)pyridin-3-yl]oxy]aniline

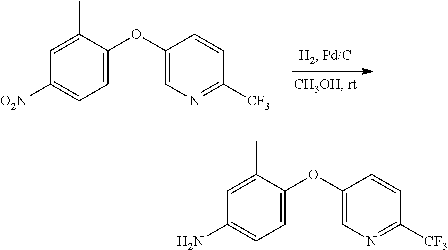

To a solution of 5-(2-methyl-4-nitrophenoxy)-2-(trifluoromethyl)pyridine (7.0 g, 23.47 mmol) in MeOH (100.0 mL) was added Pd/C (2.1 g, dry). The mixture was stirred at room temperature for 4 h under H₂. After the reaction was completed, the reaction mixture was filtered. The filtrate was evaporated in vacuo to afford 3-methyl-4-[[6-(trifluoromethyl)pyridin-3-yl]oxy]aniline (5.0 g, crude) as a light yellow oil. LCMS (ESI, m/z): [M+H]⁺=269.1.

### Step 3: Synthesis of N-(3-methyl-4-[[6-(trifluoromethyl)pyridin-3-yl]oxy]phenyl)-6-nitroquinazolin-4-amine

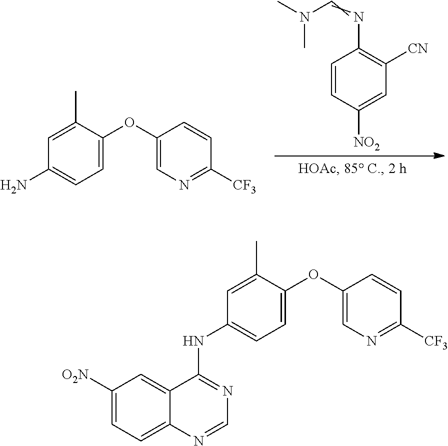 [SMILES: Cc1cc(N)ccc1Oc1ccc(C(F)(F)F)nc1 ; CN(C)C=Nc1ccc([N+](=O)[O-])cc1C#N ; Cc1cc(Nc2ncnc3ccc([N+](=O)[O-])cc23)ccc1Oc1ccc(C(F)(F)F)nc1]

To a mixture of 3-methyl-4-[[6-(trifluoromethyl)pyridin-3-yl]oxy]aniline (4.0 g, crude) in HOAc (40.0 mL) was added N′-(2-cyano-4-nitrophenyl)-N,N-dimethylformimidamide (3.2 g, 14.91 mmol) at room temperature. The resulting mixture was stirred at 85° C. for 2 h. After the reaction was completed, the mixture was purified by flash column chromatography with petroleum ether/EtOAc (1/1, v/v) to afford N-(3-methyl-4-[[6-(trifluoromethyl)pyridin-3-yl]oxy]phenyl)-6-nitroquinazolin-4-amine (4.5 g, 68%) as a yellow solid. LCMS (ESI, m/z): [M+H]⁺=442.1.

### Step 4: Synthesis of N4-(3-methyl-4-[[6-(trifluoromethyl)pyridin-3-yl]oxy]phenyl)quinazoline-4,6-diamine

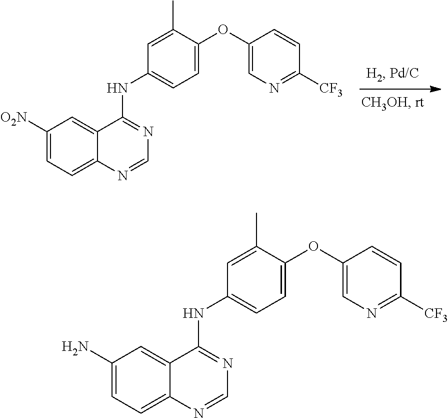 [SMILES: Cc1cc(Nc2ncnc3ccc([N+](=O)[O-])cc23)ccc1Oc1ccc(C(F)(F)F)nc1 ; Cc1cc(Nc2ncnc3ccc(N)cc23)ccc1Oc1ccc(C(F)(F)F)nc1]

To a solution of N-(3-methyl-4-[[6-(trifluoromethyl)pyridin-3-yl]oxy]phenyl)-6-nitroquinazolin-4-amine (2.0 g, 4.53 mmol) in MeOH (30.0 mL) was added Pd/C (0.6 g, dry). The mixture was stirred at room temperature for 4 h under H₂. After the reaction was completed, the reaction mixture was filtered. The filtrate was evaporated in vacuo. The residue was purified by reverse phase flash column chromatography with ACN/H₂O (60/40, v/v) to afford N4-(3-methyl-4-[[6-(trifluoromethyl)pyridin-3-yl]oxy]phenyl)quinazoline-4,6-diamine (1.4 g, 97%) as a light yellow solid. LCMS (ESI, m/z): [M+H]⁺=412.1.

### Step 5: Synthesis of N-[4-[(3-methyl-4-[[6-(trifluoromethyl)pyridin-3-yl]oxy]phenyl)amino]quinazolin-6-yl]but-2-ynamide (Compound 184)

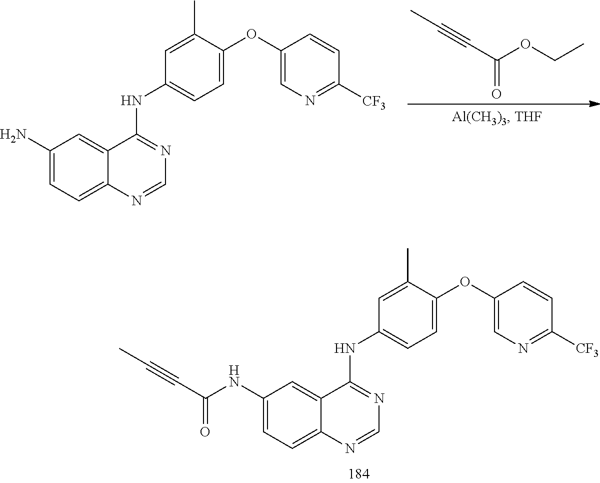

To a mixture of N4-(3-methyl-4-[[6-(trifluoromethyl)pyridin-3-yl]oxy]phenyl)quinazoline-4,6-diamine (200.0 mg, 0.48 mmol) and but-2-ynoic acid ethyl ester (163.5 mg, 1.46 mmol) in THF (10.0 mL) was added dropwise AlMe₃ (1.2 mL, 2 mol/L in toluene) at 0° C. The resulting mixture was stirred at 80° C. for 3 h. After the reaction was completed, the reaction mixture was quenched with NH₄Cl (aq) at 0° C. The resulting mixture was diluted with H₂O and extracted with DCM. The combined organic layer was washed with brine, dried over anhydrous sodium sulfate and filtered. The filtrate was concentrated under reduced pressure. The residue was purified by Prep-HPLC with the following conditions: Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 51% B to 61% B in 8 min; 254/220 nm; RT1:7.75 min to afford N-[4-[(3-methyl-4-[[6-(trifluoromethyl)pyridin-3-yl]oxy]phenyl)amino]quinazolin-6-yl]but-2-ynamide (Compound 184) (123.0 mg, 52%) as a white solid. LCMS (ESI, m/z): [M+H]⁺=478.0. ¹H NMR (300 MHz, DMSO-d₆): δ 10.95 (s, 1H), 9.88 (s, 1H), 8.77 (s, 1H), 8.55 (s, 2H), 7.90-7.74 (m, 5H), 7.42-7.38 (m, 1H), 7.17 (d, J=8.7 Hz, 1H), 2.19 (s, 3H), 2.10 (s, 3H).

### Example S74. Synthesis of N-{4-[(3-methyl-4-{[1,2,4]triazolo[1,5-a]pyridin-7-yloxy}phenyl)amino]quinazolin-6-yl}-3-(1-methylcyclopropyl)prop-2-ynamide (Compound 185)

### Step 1: Synthesis of N-{4-[(3-methyl-4-{[1,2,4]triazolo[1,5-a]pyridin-7-yloxy}phenyl)amino]quinazolin-6-yl}-3-(1-methylcyclopropyl)prop-2-ynamide (Compound 185)

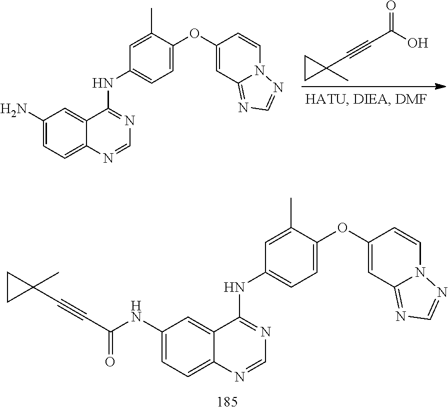

To a solution of N4-(3-methyl-4-{[1,2,4]triazolo[1,5-a]pyridin-7-yloxy}phenyl)quinazoline-4,6-diamine (300.0 mg, 0.78 mmol) in DMF (10.0 mL) was added 3-(1-methylcyclopropyl)prop-2-ynoic acid (97.1 mg, 0.78 mmol), DIEA (202.2 mg, 1.56 mmol) and HATU (357.0 mg, 0.94 mmol) at room temperature. The mixture was stirred at room temperature for 1 h. After the reaction was completed, the resulting mixture was diluted with H₂O and extracted with EtOAc. The combined organic layer was washed with brine, dried over anhydrous sodium sulfate and filtered. The filtrate was concentrated under reduced pressure. The residue was purified by Prep-HPLC with the following conditions (Column:) (Bridge Prep OBD C18 Column, 30×150 mm, 5 um; Mobile Phase A: Water (0.1% FA), Mobile Phase B: ACN; Flow rate: 60 mL/min; Gradient: 30% B to 35% B in 12 min, 254 nm) to afford N-{4-[(3-methyl-4-{[1,2,4]triazolo[1,5-a]pyridin-7-yloxy}phenyl)amino]quinazolin-6-yl}-3-(1-methylcyclopropyl)prop-2-ynamide (Compound 185) (5.2 mg, 1%) as a white solid LCMS (ESI, m/z): [M+H^{]}+=490.3. ¹H NMR (400 MHz, DMSO-d₆): δ 10.82 (s, 1H), 9.87 (s, 1H), 8.94 (d, J=7.6 Hz, 1H), 8.71 (s, 1H), 8.56 (s, 1H), 8.39 (s, 1H), 7.84-7.76 (m, 4H), 7.21 (d, J=8.4 Hz, 1H), 7.05-7.03 (m, 1H), 6.81 (d, J=2.4 Hz, 1H), 2.19 (s, 3H), 1.35 (s, 3H), 1.06 (d, J=2.4 Hz, 2H), 0.86 (d, J=2.4 Hz, 2H).

### Example S75. Synthesis of N-{7-[2-(dimethylamino)ethoxy]-4-([3-methyl-4-[(6-methylpyridin-3-yl)oxy]phenyl]amino)quinazolin-6-yl}but-2-ynamide (Compound 186)

### Step 1: Synthesis of 2-methyl-5-(2-methyl-4-nitrophenoxy)pyridine

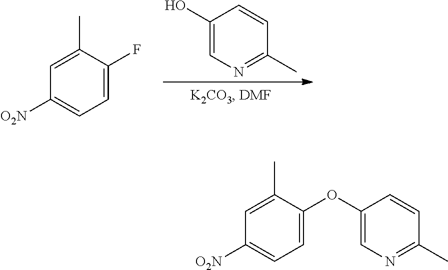

To a solution of 6-methylpyridin-3-ol (3.2 g, 29.01 mmol) in DMF (80.0 mL) was added 1-fluoro-2-methyl-4-nitrobenzene (9.0 g, 58.02 mmol) and K₂CO₃ (12.0 g, 87.03 mmol) at room temperature. The resulting mixture was stirred at 50° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/EtOAc (1/1, v/v) to afford 2-methyl-5-(2-methyl-4-nitrophenoxy)pyridine (9.0 g, 64%) as a white solid. LCMS (ESI, m/z): [M+H]⁺=245.1.

### Step 2: Synthesis of 3-methyl-4-[(6-methylpyridin-3-yl)oxy]aniline

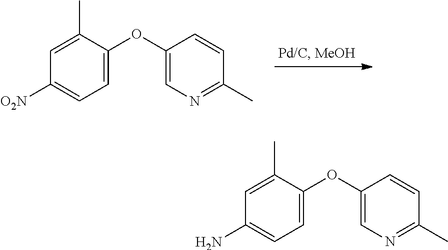

To a solution of 2-methyl-5-(2-methyl-4-nitrophenoxy)pyridine (8.8 g, 36.03 mmol) in MeOH (100.0 mL) was added Pd/C (2.6 g, dry) at room temperature. The resulting mixture was stirred at room temperature for 3 h under H₂. After the reaction was completed, the reaction mixture was filtered. The filtrate was concentrated under reduced pressure. The residue was purified by flash column chromatography with CH₂Cl₂/MeOH (10/1, v/v) to afford 3-methyl-4-[(6-methylpyridin-3-yl)oxy]aniline (8.0 g, 94%) as a yellow solid. LCMS (ESI, m/z): [M+H]⁺=215.1.

### Step 3: Synthesis of 7-[2-(dimethylamino)ethoxy]-N-{3-methyl-4-[(6-methyl pyridin-3-yl)oxy]phenyl}-6-nitroquinazolin-4-amine

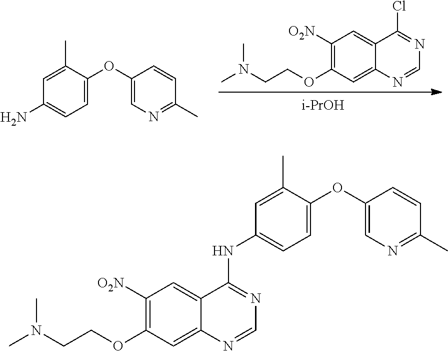

To a solution of {2-[(4-chloro-6-nitroquinazolin-7-yl)oxy]ethyl}dimethylamine (1.0 g, crude) in isopropyl alcohol (20.0 mL) was added 3-methyl-4-[(6-methylpyridin-3-yl)oxy]aniline (0.7 g, 3.37 mmol) at room temperature. The resulting mixture was stirred at room temperature for 3 h. After the reaction was completed, the reaction mixture was concentrated under reduced pressure. The residue was purified by flash column chromatography with petroleum ether/EtOAc (1/1, v/v) to afford 7-[2-(dimethylamino)ethoxy]-N-{3-methyl-4-[(6-methylpyridin-3-yl)oxy]phenyl}-6-nitroquinazolin-4-amine (1.0 g, 63%) as a yellow solid. LCMS (ESI, m/z): [M+H]⁺=475.2.

### Step 4: Synthesis of 7-[2-(dimethylamino)ethoxy]-N4-{3-methyl-4-[(6-methylpyridin-3-yl)oxy]phenyl}quinazoline-4,6-diamine

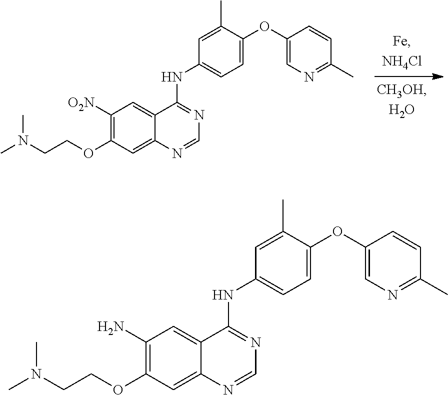

To a solution of 7-[2-(dimethylamino)ethoxy]-N-{3-methyl-4-[(6-methylpyridin-3-yl)oxy]phenyl}-6-nitroquinazolin-4-amine (950.0 mg, 2.00 mmol) in EtOH (15.0 mL)/H₂O (3.0 mL) was added NH₄Cl (318.6 mg, 6.01 mmol). Then Fe (336.7 mg, 6.01 mmol) was added to the mixture at 80° C. The resulting mixture was stirred at 80° C. for 3 h. After the reaction was completed, the reaction mixture was filtered. The filtrate was concentrated under reduced pressure. The residue was purified by flash column chromatography with CH₂Cl₂/MeOH (5/1, v/v) to afford 7-[2-(dimethylamino)ethoxy]-N4-{3-methyl-4-[(6-methylpyridin-3-yl)oxy]phenyl}quinazoline-4,6-diamine (500.0 mg, 56%) as an orange solid. LCMS (ESI, m/z): [M+H]⁺=445.2.

### Step 5: Synthesis of N-{7-[2-(dimethylamino)ethoxy]-4-({3-methyl-4-[(6-methylpyridin-3-yl)oxy]phenyl}amino)quinazolin-6-yl}but-2-ynamide (Compound 186)

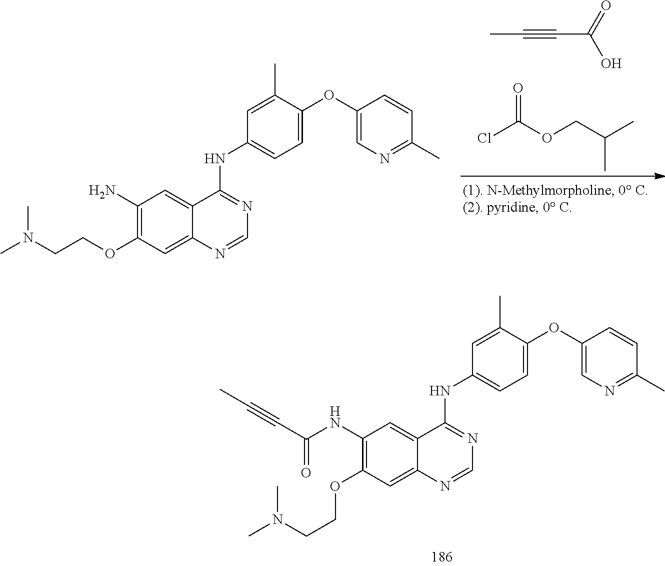

To a solution of 2-butynoic acid (85.1 mg, 1.01 mmol) in THF (10.0 mL) was added 2-methylpropyl carbonochloridate (278.0 mg, 1.98 mmol) at 0° C. under N₂. The resulting mixture was stirred at 0° C. for 5 min. Then N-Methylmorpholine (273.0 mg, 2.70 mmol) was added to the mixture at 0° C. under N₂. The mixture was stirred at 0° C. for 10 min. Then a solution of 7-[2-(dimethylamino)ethoxy]-N4-{3-methyl-4-[(6-methylpyridin-3-yl)oxy]phenyl}quinazoline-4,6-diamine (150.0 mg, 0.34 mmol) in pyridine (5.0 mL) was added dropwise to the mixture at 0° C. The mixture was stirred at 0° C. for 20 min. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: YMC-Actus Triart C₁₈ ExRS, 30×150 mm, 5 um; Mobile Phase A: Water (10 mmol/L NH₄HCO₃), Mobile Phase B: ACN; Flow rate: 60 mL/min; Gradient: 38% B to 48% B in 8 min, 254 nm) to afford N-{7-[2-(dimethylamino)ethoxy]-4-({3-methyl-4-[(6-methylpyridin-3-yl)oxy]phenyl}amino)quinazolin-6-yl}but-2-ynamide (Compound 186) (6.2 mg, 4%) as a white solid. LCMS (ESI, m/z): [M+H]⁺=511.4. ¹H NMR (400 MHz, DMSO-d₆): δ 10.24 (s, 1H), 9.70 (s, 1H), 8.70 (s, 1H), 8.50 (s, 1H), 8.19 (d, J=2.4 Hz, 1H), 7.75 (s, 1H), 7.67 (d, J=8.4 Hz, 1H), 7.33 (s, 1H), 7.26-7.19 (m, 2H), 6.96 (d, J=8.8 Hz, 1H), 4.30-4.27 (m, 2H), 2.73-2.68 (m, 2H), 2.45 (s, 3H), 2.28 (s, 6H), 2.21 (s, 3H), 2.08 (s, 3H).

### Example S76. 3-cyclopropyl-N-{4-[(3-methyl-4-{[1,2,4]triazolo[1,5-a]pyridin-7-yloxy}phenyl)amino]-7-[2-(methylamino)ethoxy]quinazolin-6-yl}prop-2-ynamide (Compound 187)

### Step 1: Synthesis of tert-butyl N-(2-{[6-(3-cyclopropylprop-2-ynamido)-4-[(3-methyl-4-{[1,2,4]triazolo[1,5-a]pyridin-7-yloxy}phenyl)amino]quinazolin-7-yl]oxy}ethyl)-N-methylcarbamate

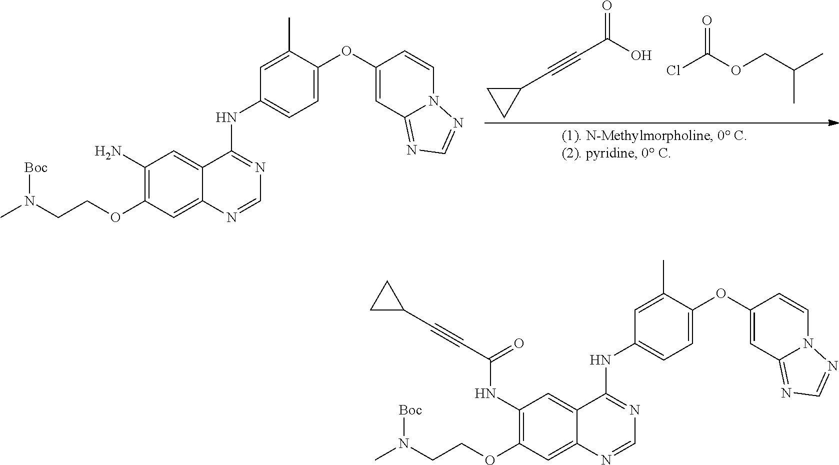

To a solution of 3-cyclopropylprop-2-ynoic acid (178.0 mg, 1.62 mmol) in THF (10.0 mL) was added 2-methylpropyl carbonochloridate (432.8 mg, 3.17 mmol) at 0° C. The resulting mixture was stirred at 0° C. for 5 min. Then N-Methylmorpholine (436.1 mg, 4.31 mmol) was added to the mixture at 0° C. The mixture was stirred at 0° C. for 30 min. Then a solution of tert-butyl N-[2-({6-amino-4-[(3-methyl-4-{[1,2,4]triazolo[1,5-a]pyridin-7-yloxy}phenyl)amino]quinazolin-7-yl}oxy)ethyl]-N-methylcarbamate (300.0 mg, 0.54 mmol) in pyridine (2.0 mL) was added dropwise to the mixture at 0° C. The mixture was stirred at 0° C. for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (5/1, v/v) to afford tert-butyl N-(2-{[6-(3-cyclopropylprop-2-ynamido)-4-[(3-methyl-4-{[1,2,4]triazolo[1,5-a]pyridin-7-yloxy}phenyl)amino]quinazolin-7-yl]oxy}ethyl)-N-methylcarbamate (110.0 mg, 31%) as a yellow solid. LCMS (ESI, m/z): [M+H]⁺=649.3.

### Step 2: Synthesis of 3-cyclopropyl-N-{4-[(3-methyl-4-{[1,2,4]triazolo[1,5-a]pyridin-7-yloxy}phenyl)amino]-7-[2-(methylamino)ethoxy]quinazolin-6-yl}prop-2-ynamide (Compound 187)

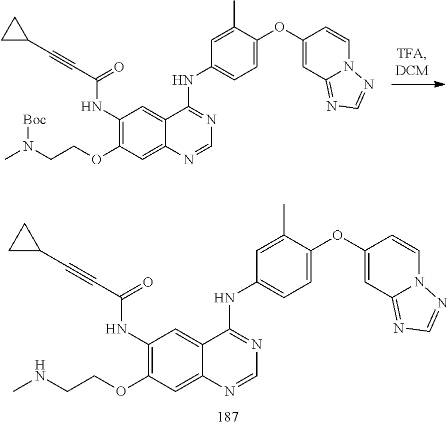 [SMILES: Cc1cc(Nc2ncnc3cc(OCCN(C)C(=O)OC(C)(C)C)c(NC(=O)C#CC4CC4)cc23)ccc1Oc1ccn2ncnc2c1 ; CNCCOc1cc2ncnc(Nc3ccc(Oc4ccn5ncnc5c4)c(C)c3)c2cc1NC(=O)C#CC1CC1]

To a mixture of tert-butyl N-(2-{[6-(3-cyclopropylprop-2-ynamido)-4-[(3-methyl-4-{[1,2,4]triazolo[1,5-a]pyridin-7-yloxy}phenyl)amino]quinazolin-7-yl]oxy}ethyl)-N-methylcarbamate (100.0 mg, 0.15 mmol) in DCM (4.0 mL) was added TFA (2.0 mL) at room temperature. The resulting mixture was stirred at room temperature for 1 h. After the reaction was completed, the mixture was basified to pH=8 with saturated NaHCO₃(aq.). The resulting mixture was diluted with H₂O and extracted with DCM. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 25% B to 36% B in 8 min; Wave Length: 254 nm) to afford 3-cyclopropyl-N-{4-[(3-methyl-4-{[1,2,4]triazolo[1,5-a]pyridin-7-yloxy}phenyl)amino]-7-[2-(methylamino)ethoxy]quinazolin-6-yl}prop-2-ynamide (Compound 187) (29.7 mg, 35%) as a white solid. LCMS (ESI, m/z): [M+H]+=549.4. ¹H NMR (400 MHz, DMSO-d₆): δ 9.72 (s, 1H), 8.93 (d, J=7.6 Hz, 1H), 8.72 (s, 1H), 8.53 (s, 1H), 8.38 (s, 1H), 7.83-7.79 (m, 2H), 7.29 (s, 1H), 7.23-7.20 (m, 1H), 7.04-7.02 (m, 1H), 6.80 (d, J=2.4 Hz, 1H), 4.26-4.23 (m, 2H), 2.91-2.89 (m, 2H), 2.39 (s, 3H), 2.19 (s, 3H), 1.64-1.56 (m, 1H), 1.02-0.79 (m, 4H).

### Example S77. (R)-3-cyclopropyl-N-(4-((4-((6-(1,2-difluoroethyl)pyridin-3-yl)oxy)−3-methylphenyl)amino)quinazolin-6-yl)propiolamide (Compound 188)

### Step 1: Synthesis of (5)-1-(5-(2-methyl-4-((6-nitroquinazolin-4-yl)amino)phenoxy)pyridin-2-yl)ethane-1,2-diol

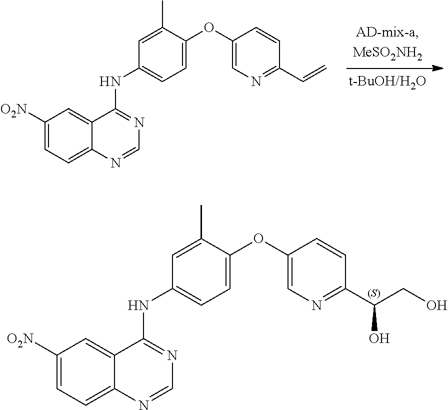

To a solution of N-[4-[(6-ethenylpyridin-3-yl)oxy]-3-methylphenyl]-6-nitroquinazolin-4-amine (9.0 g, 22.5 mmol) in H₂O (45.0 mL) and t-BuOH (45.0 mL) was added methanesulfonamide (6.4 g, 67.5 mmol) and AD-mix-a (31.5 g, 40.59 mmol). The resulting mixture was stirred at room temperature for 16 h. After the reaction was completed, the reaction mixture was quenched with water and extracted with ethyl acetate. The combined organic layer was dried over anhydrous sodium sulfate and filtered. The filtrate was concentrated under vacuum. The residue was purified by flash column chromatography with MeOH/DCM (1/9, v/v) to afford (S)-1-(5-(2-methyl-4-((6-nitroquinazolin-4-yl)amino)phenoxy)pyridin-2-yl)ethane-1,2-diol (4.0 g, 41%) as a yellow solid. LCMS (ESI, m/z): [M+H]⁺=434.1.

### Step 2: Synthesis of (R)—N-(4-((6-(1,2-difluoroethyl)pyridin-3-yl)oxy)-3-methylphenyl)-6-nitroquinazolin-4-amine

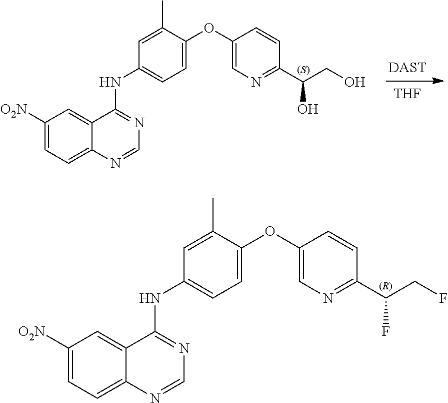

To a solution of (S)-1-(5-(2-methyl-4-((6-nitroquinazolin-4-yl)amino)phenoxy)pyridin-2-yl)ethane-1,2-diol (3.3 g, 7.61 mmol) in THF (50.0 mL) was added diethylaminosulfur trifluoride (DAST) (3.7 g, 22.84 mmol). The resulting mixture was stirred at room temperature for 16 h. After the reaction was completed, the reaction mixture was quenched with water and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (40/60, v/v) to afford (R)—N-(44(6-(1,2-difluoroethyl)pyridin-3-yl)oxy)-3-methylphenyl)-6-nitroquinazolin-4-amine (600.0 mg, 18%) as a yellow solid. LCMS (ESI, m/z): [M+H]⁺=438.1.

### Step 3: Synthesis of (R)-N4-(4-((6-(1,2-difluoroethyl)pyridin-3-yl)oxy)-3-methylphenyl)quinazoline-4,6-diamine

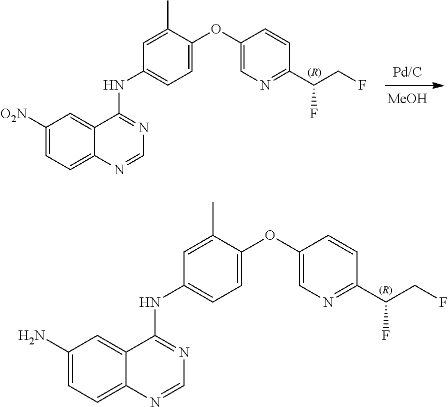

To a solution of (R)—N-(4-((6-(1,2-difluoroethyl)pyridin-3-yl)oxy)-3-methylphenyl)-6-nitroquinazolin-4-amine (600.0 mg, 1.37 mmol) in MeOH (10.0 mL) was added Pd/C (120.0 mg, dry) at room temperature. The resulting mixture was stirred at room temperature for 2 h under H₂. After the reaction was completed, the resulting mixture was filtered. The filtrate was concentrated under reduced pressure to afford (R)-N4-(44(6-(1,2-difluoroethyl)pyridin-3-yl)oxy)-3-methylphenyl)quinazoline-4,6-diamine (500.0 mg, crude) as a yellow solid. LCMS (ESI, m/z): [M+H]⁺=408.2.

### Step 4: Synthesis of (R)-3-cyclopropyl-N-(4-((4-((6-(1,2-difluoroethyl)pyridin-3-yl)oxy)-3-methylphenyl)amino)quinazolin-6-yl)propiolamide (Compound 188)

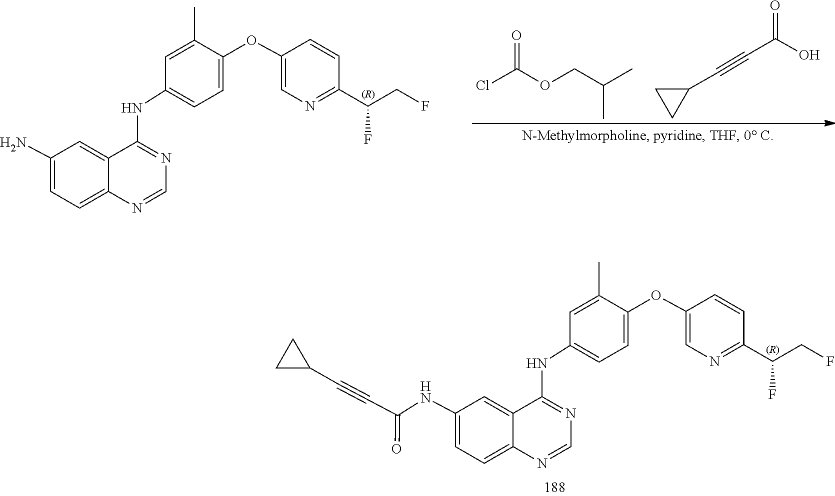

To a solution of 3-cyclopropylprop-2-ynoic acid (675.5 mg, 6.25 mmol) in THF (5.0 mL) was added 2-methylpropyl carbonochloridate (840.0 mg, 6.25 mmol) at 0 C under N2. The resulting mixture was stirred at 0 C for 5 min. Then N-Methylmorpholine (620.0 mg, 6.25 mmol) was added to the mixture at 0 C. The resulting mixture was stirred at 0 C for another 10 min. Then a solution of (R)—N⁴-(4-((6-(1,2-difluoroethyl)pyridin-3-yl)oxy)-3-methylphenyl)quinazoline-4,6-diamine (500.0 mg, 12.5 mmol) in pyridine (10.0 mL) was added dropwise to the mixture at 0 C. The resulting mixture was stirred at room temperature for 16 h. After the reaction was completed, the reaction mixture was diluted with water and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (1/9, v/v) and then purified by Prep-HPLC with the following conditions (Column: XBridge Prep OBD C18 Column, 30×150 mm, 5 um; Mobile Phase A: Water (0.1% FA), Mobile Phase B: ACN; Flow rate: 60 mL/min; Gradient: 20% B to 45% B in 8 min; 254 nm) to afford (R)-3-cyclopropyl-N-(4-((4-((6-(1,2-difluoroethyl)pyridin-3-yl)oxy)-3-methylphenyl)amino)quinazolin-6-yl)propiolamide (Compound 188) (7.3 mg, 6%) as a yellow solid. LCMS (ESI, m/z): [M+H]⁺=500.4. ¹H NMR (300 MHz, DMSO-d₆): δ 10.89 (s, 1H), 9.86 (s, 1H), 8.75 (s, 1H), 8.54 (s, 1H), 8.38 (d, J=2.4 Hz, 1H), 7.85-7.72 (m, 4H), 7.56 (d, J=9.0 Hz, 1H), 7.38-7.34 (m, 1H), 7.08 (d, J=8.7 Hz, 1H), 6.01-5.78 (m, 1H), 5.03-4.77 (m, 2H), 2.21 (s, 3H), 1.63-1.60 (m, 1H), 1.02-0.94 (m, 2H), 0.88-0.82 (m, 2H).

### Example S78. N-{7-[3-(dimethylamino)propoxy]-4-[(3-methyl-4-{[1,2,4]triazolo[1,5-a]pyridin-7-ylmethyl}phenyl)amino]quinazolin-6-yl}but-2-ynamide (Compound 189)

### Step 1: Synthesis of {3-[(4-chloro-6-nitroquinazolin-7-yl)oxy]propyl}dimethylamine

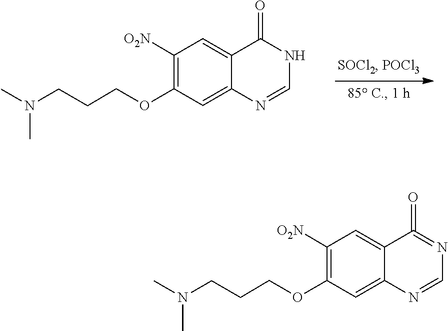

To a solution of 7-[3-(dimethylamino)propoxy]-6-nitro-3H-quinazolin-4-one (900.0 mg, 3.08 mmol) in SOCl₂ (15.0 mL) was added POCl₃ (1.0 mL) and DMF (0.2 mL) at room temperature. The resulting mixture was stirred at 85° C. for 1 h. After the reaction was completed, the mixture was concentrated under vacuum to afford {3-[(4-chloro-6-nitroquinazolin-7-yl)oxy]propyl}dimethylamine (900.0 mg, crude) as a brown yellow solid. LCMS (ESI, m/z): [M+H]⁺=311.1.

### Step 2: Synthesis of 7-[3-(dimethylamino)propoxy]-N-(3-methyl-4-{[1,2,4]triazolo[1,5-a]pyridin-7-ylmethyl}phenyl)-6-nitroquinazolin-4-amine

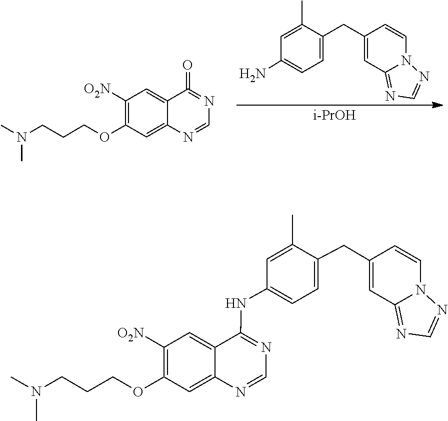

To a solution of 3-methyl-4-{[1,2,4]triazolo[1,5-a]pyridin-7-ylmethyl}aniline (0.8 g, 3.22 mmol) in i-PrOH (20.0 mL) was added {3-[(4-chloro-6-nitroquinazolin-7-yl)oxy]propyl}dimethylamine (900.0 mg, crude) at room temperature. The resulting mixture was stirred at room temperature for 2 h. After the reaction was completed, the resulting mixture was concentrated under reduced pressure. The residue was purified by flash column chromatography with CH₂Cl₂/MeOH (5/1, v/v) to afford 7-[3-(dimethylamino)propoxy]-N-(3-methyl-4-{[1,2,4]triazolo[1,5-a]pyridin-7-ylmethyl}phenyl)-6-nitroquinazolin-4-amine (800.0 mg, 48%) as a yellow solid. LCMS (ESI, m/z): [M+H]⁺=513.2.

### Step 3: Synthesis of 7-[3-(dimethylamino)propoxy]-N4-(3-methyl-4-{[1,2,4]triazolo[1,5-a]pyridin-7-ylmethyl}phenyl)quinazoline-4,6-diamine

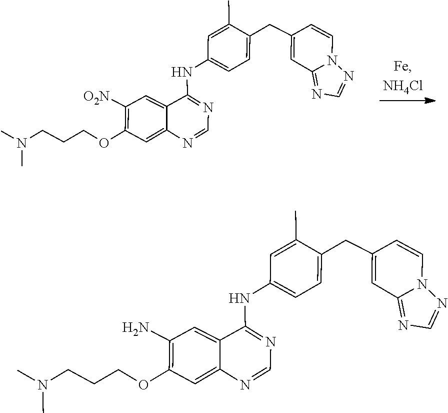 [SMILES: Cc1cc(Nc2ncnc3cc(OCCCN(C)C)c([N+](=O)[O-])cc23)ccc1Cc1ccn2ncnc2c1 ; Cc1cc(Nc2ncnc3cc(OCCCN(C)C)c(N)cc23)ccc1Cc1ccn2ncnc2c1]

To a mixture of 7-[3-(dimethylamino)propoxy]-N-(3-methyl-4-{[1,2,4]triazolo[1,5-a]pyridin-7-ylmethyl}phenyl)-6-nitroquinazolin-4-amine (450.0 mg, 0.89 mmol) and NH₄Cl (140.9 mg, 2.63 mmol) in EtOH (20.0 mL)/H₂O (4.0 mL) was added Fe (147.1 mg, 2.63 mmol) at 80° C. The resulting mixture was stirred at 80° C. for 3 h. After the reaction was completed, the resulting mixture was filtered. The filtrate was concentrated under reduced pressure. The residue was purified by flash column chromatography with CH₂Cl₂/MeOH (5/1, v/v) to afford 7-[3-(dimethylamino)propoxy]-N4-(3-methyl-4-{[1,2,4]triazolo[1,5-a]pyridin-7-ylmethyl}phenyl)quinazoline-4,6-diamine (400.0 mg, 94%) as a yellow solid. LCMS (ESI, m/z): [M+H]⁺=483.3.

### Step 4: Synthesis of N-{7-[3-(dimethylamino)propoxy]-4-[(3-methyl-4-{[1,2,4]triazolo[1,5-a]pyridin-7-ylmethyl}phenyl)amino]quinazolin-6-yl}but-2-ynamide (Compound 189)

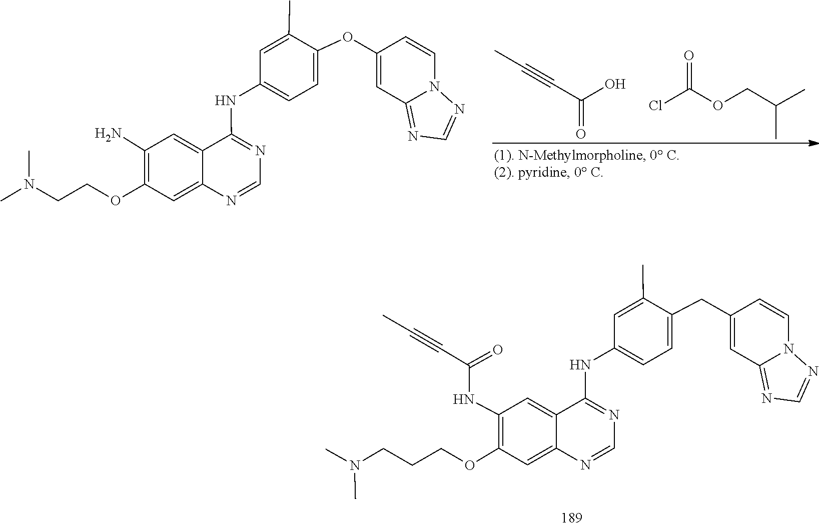

To a solution of 2-butynoic acid (41.8 mg, 0.50 mmol) in THF (10.0 mL) was added 2-methylpropyl carbonochloridate (67.9 mg, 0.50 mmol) at 0° C. The resulting mixture was stirred at 0° C. for 5 min. Then N-Methylmorpholine (125.8 mg, 1.24 mmol) was added to the mixture at 0° C. The mixture was stirred at 0° C. for 30 min. Then a solution of 7-[3-(dimethylamino)propoxy]-N4-(3-methyl-4-{[1,2,4]triazolo[1,5-a]pyridin-7-ylmethyl}phenyl)quinazoline-4,6-diamine (200.0 mg, 0.41 mmol) in pyridine (2.0 mL) was added to the mixture at 0° C. The mixture was stirred at 0° C. for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 22% B to 42% B in 8 min, 42% B; Wave Length: 254 nm) to afford N-{7-[3-(dimethylamino)propoxy]-4-[(3-methyl-4-{[1,2,4]triazolo[1,5-a]pyridin-7-ylmethyl}phenyl)amino]quinazolin-6-yl}but-2-ynamide (Compound 189) (58.6 mg, 25%) as a white solid. LCMS (ESI, m/z): [M+H]⁺=549.2. ¹H NMR (400 MHz, DMSO-d₆): δ 9.97 (s, 1H), 9.61 (s, 1H), 8.86 (d, J=7.2 Hz, 1H), 8.65 (s, 1H), 8.49-8.43 (m, 2H), 7.67-7.63 (m, 2H), 7.52 (s, 1H), 7.23 (d, J=6.8 Hz, 2H), 7.02 (d, J=7.2 Hz, 1H), 4.23-4.20 (m, 2H), 4.12 (s, 2H), 2.44-2.41 (m, 2H), 2.26 (s, 3H), 2.18 (s, 6H), 2.07 (s, 3H), 1.97-1.91 (m, 2H).

### Example S79. N-{7-[2-(dimethylamino)ethoxy]-4-[(3-methyl-4-{[1,2,4]triazolo[1,5-a]pyridin-7-yloxy}phenyl)amino]quinazolin-6-yl}-3-(1-methylcyclopropyl)prop-2-ynamide (Compound 190)

### Step 1: Synthesis of N-{7-[2-(dimethylamino)ethoxy]-4-[(3-methyl-4-{[1,2,4]triazolo[1,5-a]pyridin-7-yloxy}phenyl)amino]quinazolin-6-yl}-3-(1-methylcyclopropyl)prop-2-ynamide (Compound 190)

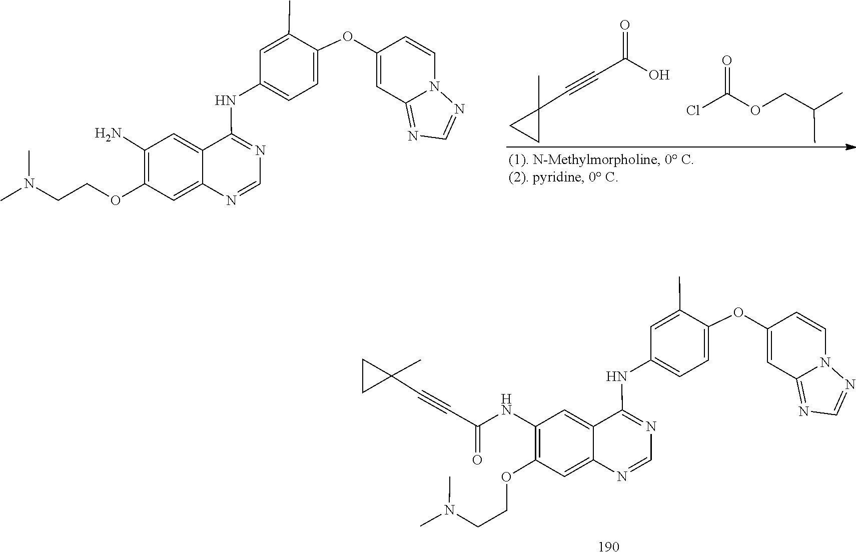

To a solution of 3-(1-methylcyclopropyl)prop-2-ynoic acid (52.8 mg, 0.42 mmol) in THF (10.0 mL) was added 2-methylpropyl carbonochloridate (58.0 mg, 0.42 mmol) at 0° C. The resulting mixture was stirred at 0° C. for 5 min. Then N-Methylmorpholine (64.5 mg, 0.64 mmol) was added to the mixture at 0° C. The mixture was stirred at 0° C. for 30 min. Then a solution of 7-[2-(dimethylamino)ethoxy]-N4-(3-methyl-4-{[1,2,4]triazolo[1,5-a]pyridin-7-yloxy}phenyl)quinazoline-4,6-diamine (200.0 mg, 0.43 mmol) in pyridine (2.0 mL) was added dropwise to the mixture at 0° C. Then mixture was stirred at 0° C. for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 30% B to 45% B in 8 min; Wave Length: 254 nm) to afford N-{7-[2-(dimethylamino)ethoxy]-4-[(3-methyl-4-{[1,2,4]triazolo[1,5-a]pyridin-7-yloxy}phenyl)amino]quinazolin-6-yl}-3-(1-methylcyclopropyl)prop-2-ynamide (Compound 190) (5.4 mg, 2%) as a white solid. LCMS (ESI, m/z): [M+H]+=577.5. ¹H NMR (400 MHz, DMSO-d₆): δ 10.14 (s, 1H), 9.75 (s, 1H), 8.94 (d, J=7.2 Hz, 1H), 8.70 (s, 1H), 8.54 (s, 1H), 8.39 (s, 1H), 7.84-7.80 (m, 2H), 7.34 (s, 1H), 7.20 (d, J=8.4 Hz, 1H), 7.05-7.02 (m, 1H), 6.80 (d, J=2.8 Hz, 1H), 4.31-4.28 (m, 2H), 2.72-2.68 (m, 2H), 2.29 (s, 6H), 2.19 (s, 3H), 1.34 (s, 3H), 1.05-1.00 (m, 2H), 0.88-0.82 (m, 2H).

### Example S80. N-{7-[2-(dimethylamino)ethoxy]-4-[(3-methyl-4-{[1,2,4]triazolo[1,5-a]pyridin-7-yloxy}phenyl)amino]quinazolin-6-yl}-4,4-dimethylpent-2-ynamide (Compound 191)

### Step 1: Synthesis of 4,4-dimethylpent-2-ynoic acid

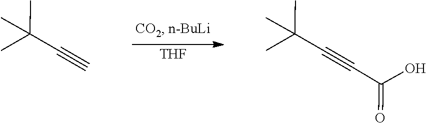

To a solution of 1-butyne, 3,3-dimethyl-(5.0 g, 60.87 mmol) in THF (100.0 mL) was added n-BuLi (26.8 mL, 2.5 mol/L) at −78° C. under N₂. The resulting mixture was stirred at −78° C. for 1 h. Then CO₂ (g) was inserted into the mixture at −78° C. The mixture was stirred at −78° C. for another 1 h. After the reaction was completed, the reaction mixture was quenched with NH₄Cl (aq) at −78° C. The resulting mixture was diluted with H₂O and extracted with ethyl ether. The combined organic layer was washed with brine, dried over anhydrous sodium sulfate and filtered. The filtrate was concentrated under vacuum to afford 4,4-dimethylpent-2-ynoic acid (7.0 g, crude) as a brown yellow oil. LCMS (ESI, m/z): [M+H]⁺=127.1.

### Step 2: Synthesis of N-{7-[2-(dimethylamino)ethoxy]-4-[(3-methyl-4-{[1,2,4]triazolo[1,5-a]pyridin-7-yloxy}phenyl)amino]quinazolin-6-yl}-4,4-dimethylpent-2-ynamide (Compound 191)

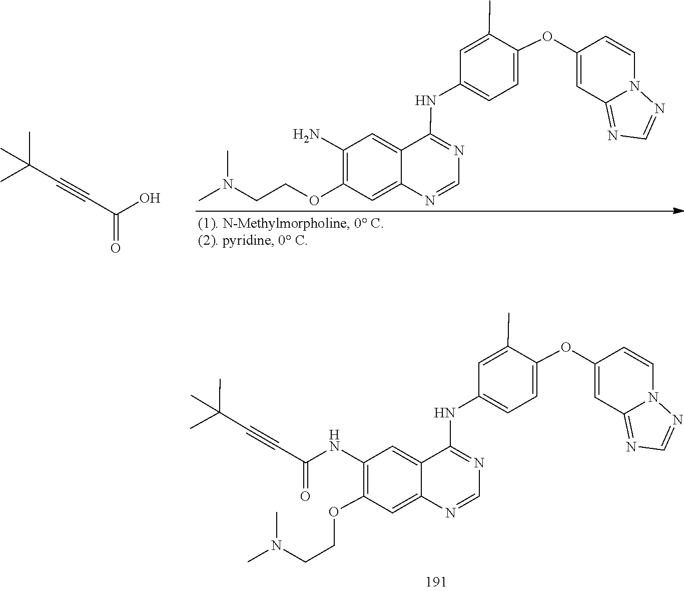

To a solution of 4,4-dimethylpent-2-ynoic acid (53.6 mg, crude) in THF (10.0 mL) was added 2-methylpropyl carbonochloridate (57.8 mg, 0.43 mmol) at 0° C. The resulting mixture was stirred at 0° C. for 5 min. Then N-Methylmorpholine (129.0 mg, 1.28 mmol) was added to the mixture at 0° C. The mixture was stirred at 0° C. for 30 min. Then a solution of 7-[2-(dimethylamino)ethoxy]-N4-(3-methyl-4-{[1,2,4]triazolo[1,5-a]pyridin-7-yloxy}phenyl)quinazoline-4,6-diamine (200.0 mg, 0.43 mmol) in pyridine (2.0 mL) was added dropwise to the mixture at 0° C. The mixture was stirred at 0° C. for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 40% B to 55% B in 8 min; Wave Length: 254 nm) to afford N-{7-[2-(dimethylamino)ethoxy]-4-[(3-methyl-4-{[1,2,4]triazolo[1,5-a]pyridin-7-yloxy}phenyl)amino]quinazolin-6-yl}-4,4-dimethylpent-2-ynamide (Compound 191) (22.4 mg, 9%) as a white solid. LCMS (ESI, m/z): [M+H]+=579.3. ¹H NMR (400 MHz, DMSO-d₆): δ 10.18 (s, 1H), 9.75 (s, 1H), 8.94 (d, J=7.2 Hz, 1H), 8.70 (s, 1H), 8.54 (s, 1H), 8.39 (s, 1H), 7.84-7.80 (m, 2H), 7.35 (s, 1H), 7.20 (d, J=8.8 Hz, 1H), 7.05-7.02 (m, 1H), 6.80 (d, J=2.4 Hz, 1H), 4.32-4.27 (m, 2H), 2.70-2.67 (m, 2H), 2.29 (s, 6H), 2.19 (s, 3H), 1.31-0.93 (m, 9H).

### Example S81. N-{4-[(3-methyl-4-{[1,2,4]triazolo[1,5-a]pyridin-7-yloxy}phenyl)amino]-7-[2-(pyrrolidin-1-yl)ethoxy]quinazolin-6-yl}but-2-ynamide (Compound 192)

### Step 1: Synthesis of 6-nitro-7-[2-(pyrrolidin-1-yl)ethoxy]-3H-quinazolin-4-one

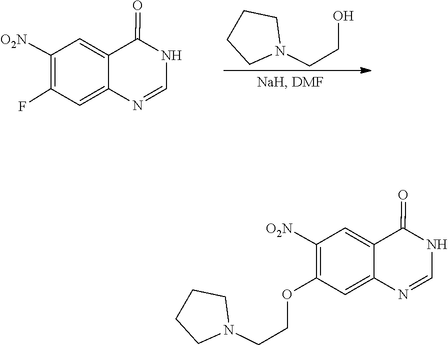 [SMILES: O=c1[nH]cnc2cc(F)c([N+](=O)[O-])cc12 ; OCCN1CCCC1 ; O=c1[nH]cnc2cc(OCCN3CCCC3)c([N+](=O)[O-])cc12]

To a solution of NaH (1.0 g, 60%) in DMF (50.0 mL) was added 2-(pyrrolidin-1-yl)ethan-1-ol (2.7 g, 23.4 mmol) at 0° C. under N₂. The mixture was stirred for 10 min at 0° C. under N₂. Then 7-fluoro-6-nitro-3H-quinazolin-4-one (4.5 g, 21.28 mmol) was added to the mixture at 0° C. under N₂. The mixture was stirred at room temperature for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (10/1, v/v) to afford 6-nitro-7-[2-(pyrrolidin-1-yl)ethoxy]-3H-quinazolin-4-one (5.0 g, 77%) as a yellow solid. LCMS (ESI, m/z): [M+H]⁺=305.1.

### Step 2: Synthesis of 4-chloro-6-nitro-7-[2-(pyrrolidin-1-yl)ethoxy]quinazoline

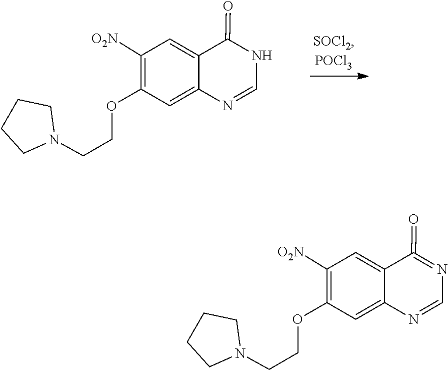

To a solution of 6-nitro-7-[2-(pyrrolidin-1-yl)ethoxy]-3H-quinazolin-4-one (1.0 g, 3.28 mmol) in SOCl₂ (15.0 mL) was added POCl₃ (1.0 mL) and DMF (0.5 mL). The reaction mixture was stirred at 90° C. for 1 h. After the reaction was completed, the mixture was concentrated under vacuo to afford 4-chloro-6-nitro-7-[2-(pyrrolidin-1-yl)ethoxy]quinazoline (1.0 g, crude) as a yellow solid. LCMS (ESI, m/z): [M+H]⁺=323.1.

### Step 3: Synthesis of N-(3-methyl-4-{[1,2,4]triazolo[1,5-a]pyridin-7-yloxy}phenyl)-6-nitro-7-[2-(pyrrolidin-1-yl)ethoxy]quinazolin-4-amine

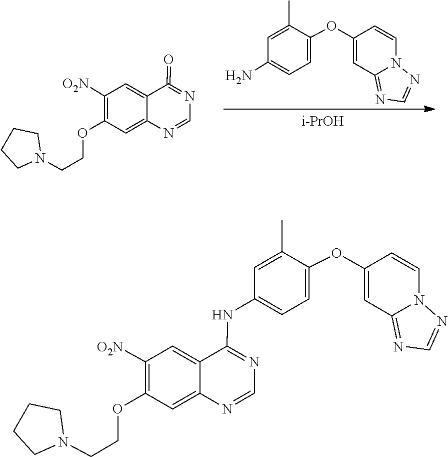

To a solution of 3-methyl-4-{[1,2,4]triazolo[1,5-a]pyridin-7-yloxy}aniline (789.0 mg, 3.18 mmol) in i-PrOH (35.0 mL) was added 4-chloro-6-nitro-7-[2-(pyrrolidin-1-yl)ethoxy]quinazoline (1.0 g, crude) at 0° C. The mixture was stirred at 0° C. for 1 h. After the reaction was completed, the resulting mixture concentrated under reduced pressure. The residue was purified by flash column chromatography with CH₂Cl₂/CH₃OH (10/1, v/v) to afford N-(3-methyl-4-{[1,2,4]triazolo[1,5-a]pyridin-7-yloxy}phenyl)-6-nitro-7-[2-(pyrrolidin-1-yl)ethoxy]quinazolin-4-amine (800.0 mg, 16%) as a yellow solid. LCMS (ESI, m/z): [M+H]⁺=527.2.

### Step 4: Synthesis of N4-(3-methyl-4-{[1,2,4]triazolo[1,5-a]pyridin-7-yloxy}phenyl)-7-[2-(pyrrolidin-1-yl)ethoxy]quinazoline-4,6-diamine

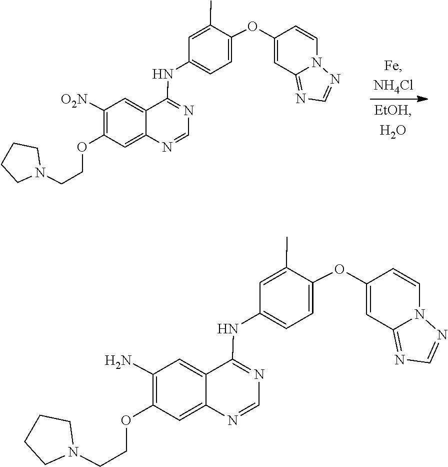 [SMILES: Cc1cc(Nc2ncnc3cc(OCCN4CCCC4)c([N+](=O)[O-])cc23)ccc1Oc1ccn2ncnc2c1 ; Cc1cc(Nc2ncnc3cc(OCCN4CCCC4)c(N)cc23)ccc1Oc1ccn2ncnc2c1]

To a solution of N-(3-methyl-4-{[1,2,4]triazolo[1,5-a]pyridin-7-yloxy}phenyl)-6-nitro-7-[2-(pyrrolidin-1-yl)ethoxy]quinazolin-4-amine (750.0 mg, 1.42 mmol) in ethanol (15.0 mL) and H₂O (3.0 mL) were added NH₄Cl (381.0 mg, 7.12 mmol) and Fe (397.7 mg, 7.12 mmol). The mixture was stirred at 80° C. for 3 h. After the reaction was completed, the resulting mixture was filtered. The filtrate was concentrated under reduced pressure. The residue was purified by flash column chromatography with CH₂Cl₂/CH₃OH (10/1, v/v) to afford N4-(3-methyl-4-{[1,2,4]triazolo[1,5-a]pyridin-7-yloxy}phenyl)-7-[2-(pyrrolidin-1-yl)ethoxy]quinazoline-4,6-diamine (300.0 mg, 42%) as a yellow solid. LCMS (ESI, m/z): [M+H]⁺=497.2.

### Step 5: Synthesis of N-{4-[(3-methyl-4-{[1,2,4]triazolo[1,5-a]pyridin-7-yloxy}phenyl)amino]-7-[2-(pyrrolidin-1-yl)ethoxy]quinazolin-6-yl}but-2-ynamide (Compound 192)

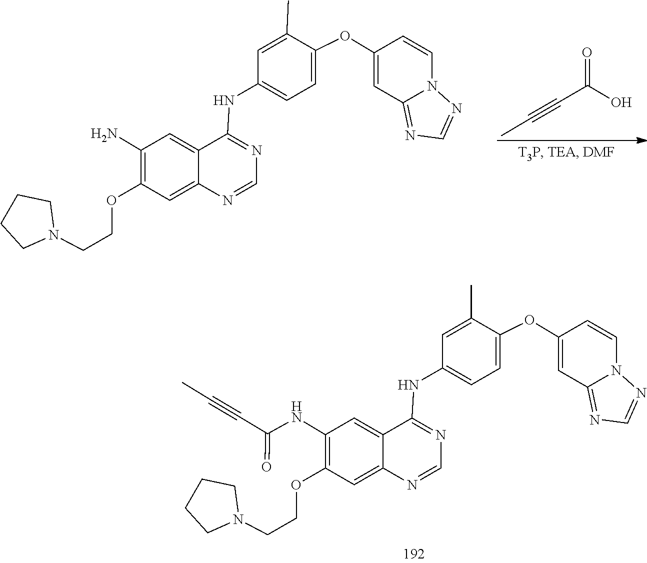

To a solution of N4-(3-methyl-4-{[1,2,4]triazolo[1,5-a]pyridin-7-yloxy}phenyl)-7-[2-(pyrrolidin-1-yl)ethoxy]quinazoline-4,6-diamine (200.0 mg, 0.52 mmol) in DMF (5.0 mL) was added 2-butynoic acid (67.7 mg, 0.81 mmol), TEA (81.5 mg, 0.81 mmol) and T3P (512.6 mg, 50%) at room temperature. The mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 20% B to 35% B in 8 min; Wave Length: 254 nm) to afford N-{4-[(3-methyl-4-{[1,2,4]triazolo[1,5-a]pyridin-7-yloxy}phenyl)amino]-7-[2-(pyrrolidin-1-yl)ethoxy]quinazolin-6-yl}but-2-ynamide (Compound 192) (2.6 mg, 1%) as a white solid. LCMS (ESI, m/z): [M+H]⁺=563.4. 1H NMR (400 MHz, DMSO-d₆): δ 10.06 (s, 1H), 9.75 (s, 1H), 8.93 (d, J=7.6 Hz, 1H), 8.70 (s, 1H), 8.54 (s, 1H), 8.38 (s, 1H), 7.84-7.80 (m, 2H), 7.33 (s, 1H), 7.20 (d, J=8.4 Hz, 1H), 7.04-7.02 (m, 1H), 6.80 (d, J=2.4 Hz, 1H), 4.32-4.30 (m, 2H), 2.89-2.86 (m, 2H), 2.61-2.58 (m, 4H), 2.19 (s, 3H), 2.07 (s, 3H), 1.79-1.68 (m, 4H).

### Example S82. N-(4-((4-([1,2,4]triazolo[1,5-a]pyridin-7-yloxy)-3-chloro-2-fluorophenyl)amino)-7-(2-(dimethylamino)ethoxy)quinazolin-6-yl)but-2-ynamide (Compound 193)

### Step 1: Synthesis of 7-(2-chloro-3-fluoro-4-nitrophenoxy)-[1,2,4]triazolo[1,5-a]pyridine

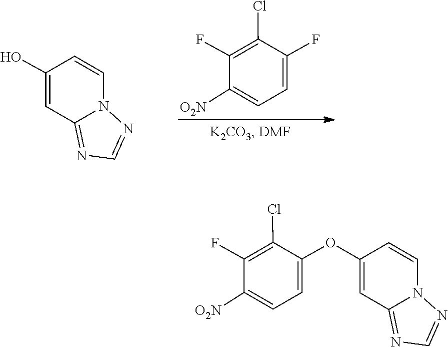 [SMILES: Oc1ccn2ncnc2c1 ; O=[N+]([O-])c1ccc(F)c(Cl)c1F ; O=[N+]([O-])c1ccc(Oc2ccn3ncnc3c2)c(Cl)c1F]

To a solution of [1,2,4]triazolo[1,5-a]pyridin-7-ol (3.0 g, 22.2 mmol) in DMF (30.0 mL) was added 2-chloro-1,3-difluoro-4-nitrobenzene (4.5 g, 24.42 mmol) and K₂CO₃ (6.0 g, 44.4 mmol) at room temperature. The resulting mixture was stirred at 70° C. for 16 h. After the reaction was completed, the mixture was diluted with water and extracted with ethyl acetate. The combined organic layers were washed with brine, dried over anhydrous Na₂SO₄ and filtered. The filtrate was concentrated under reduced pressure. The residue was purified by flash column chromatography with CH₂Cl₂/MeOH (10/1, v/v) to afford 7-(2-chloro-3-fluoro-4-nitrophenoxy)-[1,2,4]triazolo[1,5-a]pyridine (1.5 g, 21%) as yellow solid. LCMS (ESI, m/z): [M+H]⁺=309.0

### Step 2: Synthesis of 4-([1,2,4]triazolo[1,5-a]pyridin-7-yloxy)-3-chloro-2-fluoroaniline

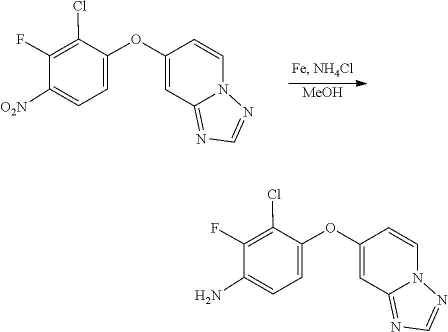 [SMILES: "MeOH" ; O=[N+]([O-])c1ccc(Oc2ccn3ncnc3c2)c(Cl)c1F ; Nc1ccc(Oc2ccn3ncnc3c2)c(Cl)c1F]

A mixture of 7-(2-chloro-3-fluoro-4-nitrophenoxy)-[1,2,4]triazolo[1,5-a]pyridine (1.5 g, 4.87 mmol), Fe (13.6 g, 24.35 mmol) and NH₄Cl (12.9 g, 24.35 mmol) in CH₃OH (20.0 mL)/H₂O (4.0 mL) was heated at 80° C. for 1 h. After the reaction was completed, the mixture was concentrated under vacuum. The residue was purified by flash column chromatography with CH₂Cl₂/MeOH (10/1, v/v) to afford 4-([1,2,4]triazolo[1,5-a]pyridin-7-yloxy)-3-chloro-2-fluoroaniline (1.0 g, 73%) as a yellow solid. LCMS (ESI, m/z): [M+H]⁺=279.0.

### Step 3: Synthesis of N-(4-([1,2,4]triazolo[1,5-a]pyridin-7-yloxy)-3-chloro-2-fluorophenyl)-7-(2-(dimethylamino)ethoxy)-6-nitroquinazolin-4-amine

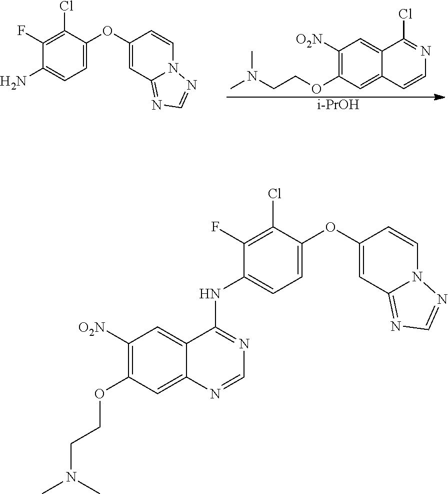

A mixture of 4-([1,2,4]triazolo[1,5-a]pyridin-7-yloxy)-3-chloro-2-fluoroaniline (1.0 g, 3.59 mmol) and 2-((4-chloro-6-nitroquinazolin-7-yl)oxy)-N,N-dimethylethan-1-amine (1.0 g, crude) in i-PrOH (20.0 mL) was stirred at 25° C. for 16 h. After the reaction was completed, the mixture was concentrated under reduced pressure. The residue was purified by flash column chromatography with CH₂Cl₂/MeOH (7/3, v/v) to afford N-(4-([1,2,4]triazolo[1,5-a]pyridin-7-yloxy)-3-chloro-2-fluorophenyl)-7-(2-(dimethylamino)ethoxy)-6-nitroquinazolin-4-amine (1.0 g, 51%) as a yellow solid. LCMS (ESI, m/z): [M+H]⁺=539.0.

### Step 4: Synthesis of N⁴-(4-([1,2,4]triazolo[1,5-a]pyridin-7-yloxy)-3-chloro-2-fluorophenyl)-7-(2-(dimethylamino)ethoxy)quinazoline-4,6-diamine

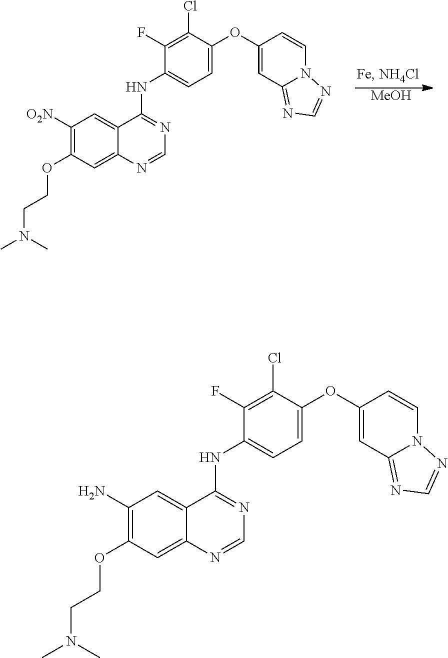 [SMILES: "MeOH" ; CN(C)CCOc1cc2ncnc(Nc3ccc(Oc4ccn5ncnc5c4)c(Cl)c3F)c2cc1[N+](=O)[O-] ; CN(C)CCOc1cc2ncnc(Nc3ccc(Oc4ccn5ncnc5c4)c(Cl)c3F)c2cc1N]

A mixture of N-(4-([1,2,4]triazolo[1,5-a]pyridin-7-yloxy)-3-chloro-2-fluorophenyl)-7-(2-(dimethylamino)ethoxy)-6-nitroquinazolin-4-amine (1.0 g, 1.85 mmol), Fe (1.0 g, 18.50 mmol) and NH₄Cl (980.5 mg, 18.50 mmol) in CH₃OH (20.0 mL)/H₂O (4.0 mL) was heated at 80° C. for 1 h. After the reaction was completed, the resulting mixture was filtered. The filtrate was concentrated under vacuum. The residue was purified by reverse phase flash column chromatography with CH₃CN/H₂O (3/1, v/v) to afford N4-(4-([1,2,4]triazolo[1,5-a]pyridin-7-yloxy)-3-chloro-2-fluorophenyl)-7-(2-(dimethylamino)ethoxy)quinazoline-4,6-diamine (600.0 mg, 63%) as a yellow solid. LCMS (ESI, m/z): [M+H]⁺=509.0

### Step 5: Synthesis of N-(4-((4-([1,2,4]triazolo[1,5-a]pyridin-7-yloxy)-3-chloro-2-fluorophenyl)amino)-7-(2-(dimethylamino)ethoxy)quinazolin-6-yl)but-2-ynamide (Compound 193)

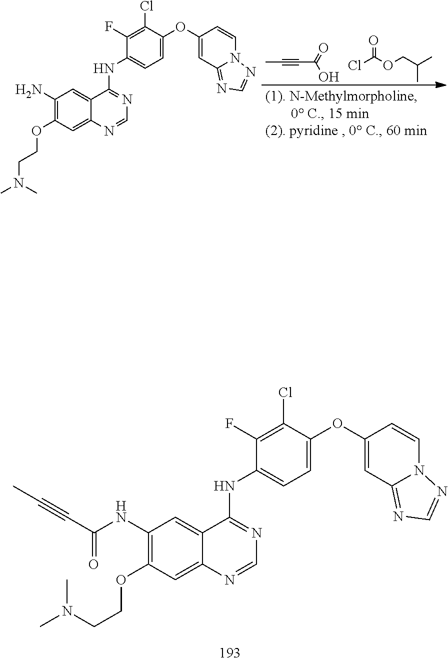

To a solution of but-2-ynoic acid (297.3 mg 3.54 mmol) in THF (10.0 mL) was added isobutyl carbonochloridate (484.9 mg, 3.54 mmol) at 0° C. The mixture was stirred at 0° C. for 5 min. To the above mixture was added dropwise N-Methylmorpholine (596.4 mg, 5.90 mmol) at 0° C. The resulting mixture was stirred at 0° C. for additional 10 min. Then a solution of N4-(4-([1,2,4]triazolo[1,5-a]pyridin-7-yloxy)-3-chloro-2-fluorophenyl)-7-(2-(dimethylamino)ethoxy)quinazoline-4,6-diamine (300.0 mg, 0.59 mmol) in pyridine (3.0 mL) was added dropwise to the mixture at 0° C. The mixture was stirred at 0° C. for additional 40 min. After the reaction was completed, the reaction was quenched with water and extracted with ethyl acetate. The combined organic layers were washed with brine, dried over anhydrous Na₂SO₄ and filtered. The filtrate was concentrated under reduced pressure. The residue was purified by reverse phase flash column chromatography with CH₃CN/H₂O (4/6, v/v) and then purified by Prep-HPLC with the following conditions Column: (XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 29% B to 40% B in 8 min, 254 nm) to afford N-(4-((4-([1,2,4]triazolo[1,5-a]pyridin-7-yloxy)-3-chloro-2-fluorophenyl)amino)-7-(2-(dimethylamino)ethoxy)quinazolin-6-yl)but-2-ynamide (Compound 193) (9.8 mg, 3%) as a white solid. LCMS (ESI, m/z): [M+H]⁺=575.3. 1H NMR (400 MHz, DMSO-d₆): δ 10.26 (s, 1H), 9.96 (s, 1H), 9.02 (d, J=7.6 Hz, 1H), 8.74 (s, 1H), 8.49-8.45 (m, 2H), 7.68-7.55 (m, 1H), 7.38-7.31 (m, 2H), 7.18-7.12 (m, 2H), 4.35-4.30 (m, 2H), 2.79-2.66 (s, 2H), 2.31 (s, 6H), 2.09 (s, 3H).

### Example S83. N-{4-[(3-methyl-4-{[1,2,4]triazolo[1,5-a]pyridin-7-yloxy}phenyl)amino]-7-[2-(methylamino)ethoxy]quinazolin-6-yl}but-2-ynamide (Compound 194)

### Step 1: Synthesis of Tert-butyl N-methyl-N-{2-[(6-nitro-4-oxo-3H-quinazolin-7-yl)oxy]ethyl}carbamate

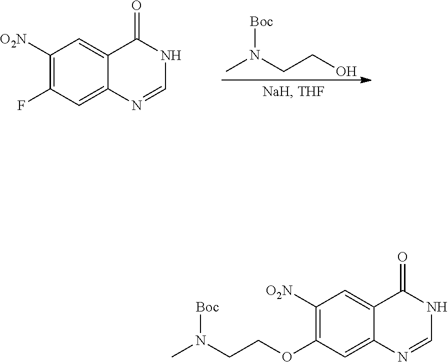 [SMILES: O=c1[nH]cnc2cc(F)c([N+](=O)[O-])cc12 ; CN(CCO)C(=O)OC(C)(C)C ; CN(CCOc1cc2nc[nH]c(=O)c2cc1[N+](=O)[O-])C(=O)OC(C)(C)C]

To a solution of 7-fluoro-6-nitro-3H-quinazolin-4-one (5.0 g, 23.91 mmol) in THF (50.0 mL) was added NaH (2.3 g, 60%) at 0° C. under N₂. The mixture was stirred at 0° C. for 30 min. Then tert-butyl N-(2-hydroxyethyl)-N-methylcarbamate (12.6 g, 71.72 mmol) was added to the mixture at 0° C. under N₂. The resulting mixture was stirred at room temperature for 1 h. After the reaction was completed, the reaction mixture was quenched with H₂O at room temperature. The mixture was concentrated under reduced pressure. The residue was purified by flash column chromatography with CH₂Cl₂/MeOH (10/1, v/v) to afford tert-butyl N-methyl-N-{2-[(6-nitro-4-oxo-3H-quinazolin-7-yl)oxy]ethyl}carbamate (6.0 g, 68%) as a white solid. LCMS (ESI, m/z): [M+H]⁺=365.1.

### Step 2: Synthesis of {2-[(4-chloro-6-nitroquinazolin-7-yl)oxy]ethyl}(methyl)amine

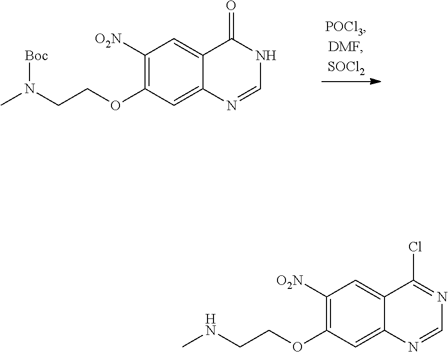 [SMILES: CN(CCOc1cc2nc[nH]c(=O)c2cc1[N+](=O)[O-])C(=O)OC(C)(C)C ; CNCCOc1cc2ncnc(Cl)c2cc1[N+](=O)[O-]]

To a solution of tert-butyl N-methyl-N-{2-[(6-nitro-4-oxo-3H-quinazolin-7-yl)oxy]ethyl}carbamate (2.0 g, 5.49 mmol) in SOCl₂ (20.0 mL) was added POCl₃ (2.0 mL) and DMF (0.4 mL) at room temperature. The resulting mixture was stirred at 85° C. for 1 h. After the reaction was completed, the mixture was concentrated under vacuum to afford {2-[(4-chloro-6-nitroquinazolin-7-yl)oxy]ethyl}(methyl)amine (1.0 g, crude) as a yellow solid. LCMS (ESI, m/z): [M+H]⁺=283.1.

### Step 3: Synthesis of N-(3-methyl-4-{[1,2,4]triazolo[1,5-a]pyridin-7-yloxy}phenyl)-7-[2-(methylamino)ethoxy]-6-nitroquinazolin-4-amine

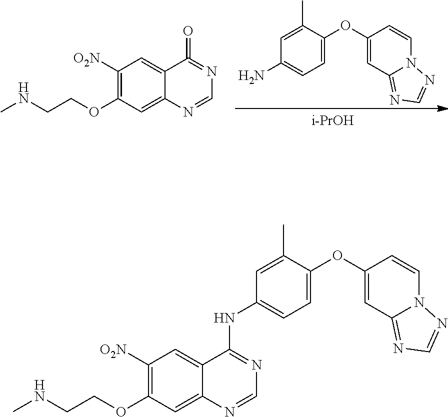

To a solution of 3-methyl-4-{[1,2,4]triazolo[1,5-a]pyridin-7-yloxy}aniline (1.3 g, 5.23 mmol) in i-PrOH (50.0 mL) was added {2-[(4-chloro-6-nitroquinazolin-7-yl)oxy]ethyl}(methyl)amine (1.5 g, crude) at room temperature. The resulting mixture was stirred at room temperature for 2 h. After the reaction was completed, the resulting mixture was concentrated under reduced pressure. The residue was purified by flash column chromatography with CH₂Cl₂/MeOH (10/1, v/v) to afford N-(3-methyl-4-{[1,2,4]triazolo[1,5-a]pyridin-7-yloxy}phenyl)-7-[2-(methylamino)ethoxy]-6-nitroquinazolin-4-amine (1.0 g, 37%) as a yellow solid. LCMS (ESI, m/z): [M+H]⁺=487.2.

### Step 4: Synthesis of Tert-butyl N-methyl-N-[2-({4-[(3-methyl-4-{[1,2,4]triazolo[1,5-a]pyridin-7-yloxy}phenyl)amino]-6-nitroquinazolin-7-yl}oxy)ethyl]carbamate

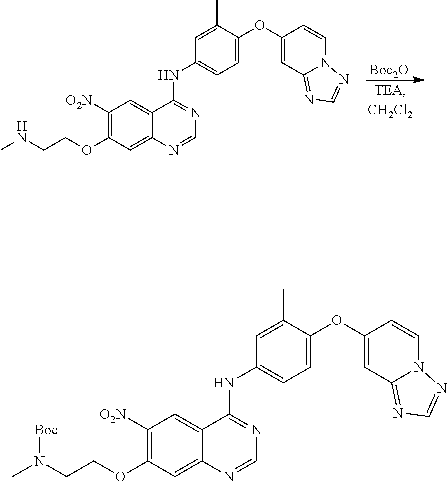 [SMILES: "Boc2O" ; CNCCOc1cc2ncnc(Nc3ccc(Oc4ccn5ncnc5c4)c(C)c3)c2cc1[N+](=O)[O-] ; Cc1cc(Nc2ncnc3cc(OCCN(C)C(=O)OC(C)(C)C)c([N+](=O)[O-])cc23)ccc1Oc1ccn2ncnc2c1]

To a solution of N-(3-methyl-4-{[1,2,4]triazolo[1,5-a]pyridin-7-yloxy}phenyl)-7-[2-(methylamino)ethoxy]-6-nitroquinazolin-4-amine (950.0 mg, 1.95 mmol) in DCM (10.0 mL) was added Boc2O (426.2 mg, 1.95 mmol) and TEA (395.2 mg, 3.91 mmol) at room temperature. The resulting mixture was stirred at room temperature for 16 h. After the reaction was completed, the resulting mixture was concentrated under reduced pressure. The mixture was purified by flash column chromatography with CH₂Cl₂/MeOH (5/1, v/v) to afford tert-butyl N-methyl-N-[2-({4-[(3-methyl-4-{[1,2,4]triazolo[1,5-a]pyridin-7-yloxy}phenyl)amino]-6-nitroquinazolin-7-yl}oxy)ethyl]carbamate (750.0 mg, 65%) as a yellow solid. LCMS (ESI, m/z): [M+H]⁺=587.2.

### Step 5: Synthesis of Tert-butyl N-[2-({6-amino-4-[(3-methyl-4-{[1,2,4]triazolo[1,5-a]pyridin-7-yloxy}phenyl)amino]quinazolin-7-yl}oxy)ethyl]-N-methylcarbamate

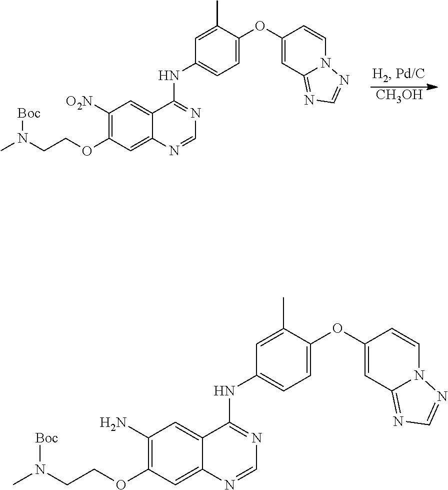

To a solution of tert-butyl N-methyl-N-[2-({4-[(3-methyl-4-{[1,2,4]triazolo[1,5-a]pyridin-7-yloxy}phenyl)amino]-6-nitroquinazolin-7-yl}oxy)ethyl]carbamate (700.0 mg, 1.19 mmol) in MeOH (40.0 mL) was added Pd/C (280.0 mg, dry). The mixture was stirred at room temperature for 16 h under H₂. After the reaction was completed, the mixture was filtered. The filtrate was evaporated in vacuo. The residue was purified by flash column chromatography with EtOAc/MeOH (10/1, v/v) to afford tert-butyl N-[2-({6-amino-4-[(3-methyl-4-{[1,2,4]triazolo[1,5-a]pyridin-7-yloxy}phenyl)amino]quinazolin-7-yl}oxy)ethyl]-N-methylcarbamate (300.0 mg, 45%) as a yellow oil. LCMS (ESI, m/z): [M+H]⁺=557.3.

### Step 6: Synthesis of Tert-butyl N-(2-{[6-(but-2-ynamido)-4-[(3-methyl-4-{[1,2,4]triazolo[1,5-a]pyridin-7-yloxy}phenyl)amino]quinazolin-7-yl]oxy}ethyl)-N-methylcarbamate

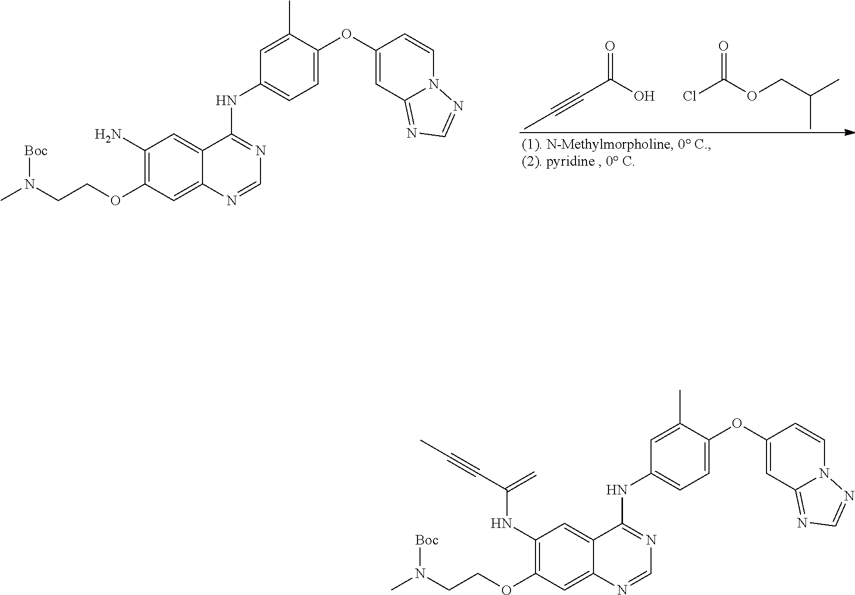

To a solution of 2-butynoic acid (117.8 mg, 1.40 mmol) in THF (10.0 mL) was added 2-methylpropyl carbonochloridate (375.1 mg, 2.74 mmol) at 0° C. The resulting mixture was stirred at 0° C. for 5 min. Then N-Methylmorpholine (377.9 mg, 3.74 mmol) was added to the mixture. The resulting mixture was stirred at 0° C. for 30 min. Then a solution of tert-butyl N-[2-({6-amino-4-[(3-methyl-4-{[1,2,4]triazolo[1,5-a]pyridin-7-yloxy}phenyl)amino]quinazolin-7-yl}oxy)ethyl]-N-methylcarbamate (260.0 mg, 0.46 mmol) in pyridine (2.0 mL) was added dropwise to the mixture. The resulting mixture was stirred at 0° C. for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (5/1, v/v) to afford tert-butyl N-(2-{[6-(but-2-ynamido)-4-[(3-methyl-4-{[1,2,4]triazolo[1,5-a]pyridin-7-yloxy}phenyl)amino]quinazolin-7-yl]oxy}ethyl)-N-methylcarbamate (85.0 mg, 29%) as a yellow solid. LCMS (ESI, m/z): [M+H]⁺=623.3.

### Step 7: Synthesis of N-{4-[(3-methyl-4-{[1,2,4]triazolo[1,5-a]pyridin-7-yloxy}phenyl)amino]-7-[2-(methylamino)ethoxy]quinazolin-6-yl}but-2-ynamide (Compound 194)

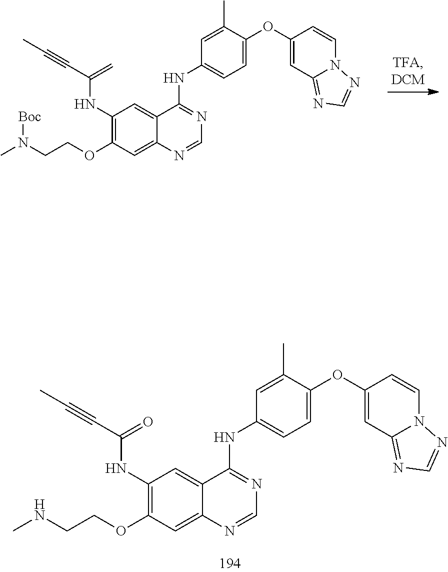 [SMILES: C=C(C#CC)Nc1cc2c(Nc3ccc(Oc4ccn5ncnc5c4)c(C)c3)ncnc2cc1OCCN(C)C(=O)OC(C)(C)C ; CC#CC(=O)Nc1cc2c(Nc3ccc(Oc4ccn5ncnc5c4)c(C)c3)ncnc2cc1OCCNC]

To a mixture of tert-butyl N-(2-{[6-(but-2-ynamido)-4-[(3-methyl-4-{[1,2,4]triazolo[1,5-a]pyridin-7-yloxy}phenyl)amino]quinazolin-7-yl]oxy}ethyl)-N-methylcarbamate (80.0 mg, 0.15 mmol) in DCM (4.0 mL) was added TFA (2.0 mL) at room temperature. The resulting mixture was stirred at room temperature for 1 h. After the reaction was completed, the mixture was basified to pH=8 with saturated NaHCO₃(aq.). The resulting mixture was diluted with H₂O and extracted with DCM. The combined organic layer was washed with brine, dried over anhydrous sodium sulfate and filtered. The filtrate was concentrated under reduced pressure. The residue was purified by Prep-HPLC with the following conditions Column (XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 21% B to 31% B in 8 min; Wave Length: 254/220 nm) to afford N-{4-[(3-methyl-4-{[1,2,4]triazolo[1,5-a]pyridin-7-yloxy}phenyl)amino]-7-[2-(methylamino)ethoxy]quinazolin-6-yl}but-2-ynamide (Compound 194) (7.4 mg, 11%) as a white solid. LCMS (ESI, m/z): [M+H]⁺=523.3. 1H NMR (400 MHz, DMSO-d₆): δ 9.75 (s, 1H), 8.94 (d, J=7.6 Hz, 1H), 8.76 (s, 1H), 8.53 (s, 1H), 8.39 (s, 1H), 7.83-7.79 (m, 2H), 7.29 (s, 1H), 7.22 (d, J=8.8 Hz, 1H), 7.05-7.02 (m, 1H), 6.80 (d, J=2.4 Hz, 1H), 4.26-4.23 (m, 2H), 2.91-2.88 (m, 2H), 2.37 (s, 3H), 2.19 (s, 3H), 2.09 (s, 3H).

### Example S84. N-{4-[(3-cyclopropyl-4-{[1,2,4]triazolo[1,5-a]pyridin-7-yloxy}phenyl)amino]-7-[2-(dimethylamino)ethoxy]quinazolin-6-yl}but-2-ynamide (Compound 195)

### Step 1: Synthesis of 2-cyclopropyl-1-fluoro-4-nitrobenzene

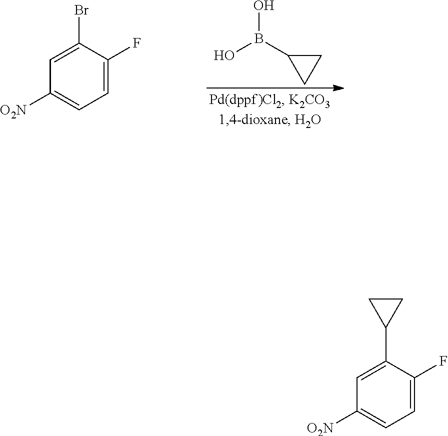 [SMILES: O=[N+]([O-])c1ccc(F)c(Br)c1 ; OB(O)C1CC1 ; O=[N+]([O-])c1ccc(F)c(C2CC2)c1]

To a mixture of 2-bromo-1-fluoro-4-nitrobenzene (1.0 g, 4.54 mmol), cyclopropylboronic acid (429.5 mg, 5.00 mmol) and K₂CO₃ (1.9 g, 13.62 mmol) in 1,4-dioxane (10.0 mL)/H₂O (2.0 mL) was added Pd(dppf)Cl₂ (665.2 mg, 0.91 mmol) at room temperature under N₂. The mixture was stirred at 80° C. for 16 h. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford 2-cyclopropyl-1-fluoro-4-nitrobenzene (500.0 mg, 60%) as a light brown oil. LCMS (ESI, m/z): [M+H]⁺=182.1.

### Step 2: Synthesis of 7-(2-cyclopropyl-4-nitrophenoxy)-[1,2,4]triazolo[1,5-a]pyridine

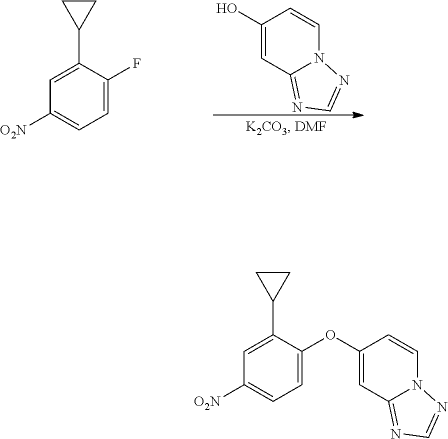 [SMILES: O=[N+]([O-])c1ccc(F)c(C2CC2)c1 ; Oc1ccn2ncnc2c1 ; O=[N+]([O-])c1ccc(Oc2ccn3ncnc3c2)c(C2CC2)c1]

To a solution of 2-cyclopropyl-1-fluoro-4-nitrobenzene (500.0 mg, 2.76 mmol) in DMF (10.0 mL) was added [1,2,4]triazolo[1,5-a]pyridin-7-ol (410.2 mg, 3.03 mmol) and K₂CO₃ (762.8 mg, 5.52 mmol) at room temperature. The mixture was stirred at 50° C. for 16 h. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (7/3, v/v) to afford 7-(2-cyclopropyl-4-nitrophenoxy)-[1,2,4]triazolo[1,5-a]pyridine (800.0 mg, 97%) as a brown oil. LCMS (ESI, m/z): [M+H]⁺=297.1

### Step 3: Synthesis of 4-([1,2,4]triazolo[1,5-a]pyridin-7-yloxy)-3-cyclopropylaniline

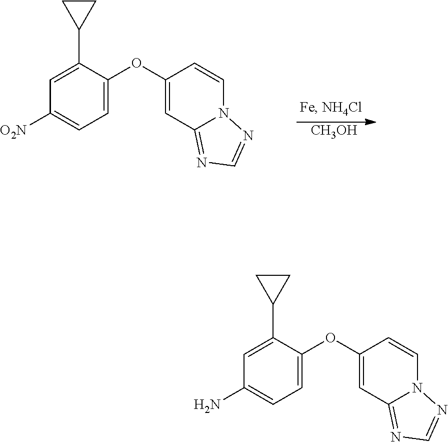 [SMILES: O=[N+]([O-])c1ccc(Oc2ccn3ncnc3c2)c(C2CC2)c1 ; "CH3OH" ; Nc1ccc(Oc2ccn3ncnc3c2)c(C2CC2)c1]

A mixture of 7-(2-cyclopropyl-4-nitrophenoxy)-[1,2,4]triazolo[1,5-a]pyridine (800.0 mg, 2.70 mmol), Fe (1.5 g, 27.00 mmol) and NH₄Cl (1.4 g, 27.00 mmol) in CH₃OH (20.0 mL)/H₂O (2.0 mL) was heated at 80° C. for 1 h. After the reaction was completed, the resulting mixture was filtered. The filtrate was concentrated under reduced pressure. The residue was purified by flash column chromatography with CH₂Cl₂/CH₃OH (7/3, v/v)) to afford 4-([1,2,4]triazolo[1,5-a]pyridin-7-yloxy)-3-cyclopropylaniline (600.0 mg, 83%) as a brown yellow oil. LCMS (ESI, m/z): [M+H]⁺=267.1.

### Step 4: Synthesis of N-(4-([1,2,4]triazolo[1,5-a]pyridin-7-yloxy)-3-cyclopropylphenyl)-7-(2-(dimethylamino)ethoxy)-6-nitroquinazolin-4-amine

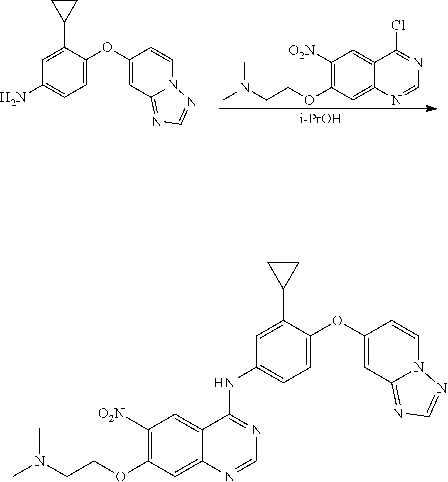

A mixture of 3-cyclopropyl-4-{[1,2,4]triazolo[1,5-a]pyridin-7-yloxy}aniline (500.0 mg, 1.87 mmol) and {2-[(4-chloro-6-nitroquinazolin-7-yl)oxy]ethyl}dimethylamine (557.0 mg, crude) in i-PrOH (20.0 mL) was stirred at room temperature for 2 h. After the reaction was completed, the mixture was concentrated under reduced pressure. The residue was purified by flash column chromatography with CH₂Cl₂/MeOH (7/3, v/v) to afford N-(3-cyclopropyl-4-{[1,2,4]triazolo[1,5-a]pyridin-7-yloxy}phenyl)-7-[2-(dimethylamino)ethoxy]-6-nitroquinazolin-4-amine (1.0 g, 91%) as a yellow solid. LCMS (ESI, m/z): [M+H]⁺=527.2.

### Step 5: Synthesis of N⁴-(4-([1,2,4]triazolo[1,5-a]pyridin-7-yloxy)-3-cyclopropylphenyl)-7-(2-(dimethylamino)ethoxy)quinazoline-4,6-diamine

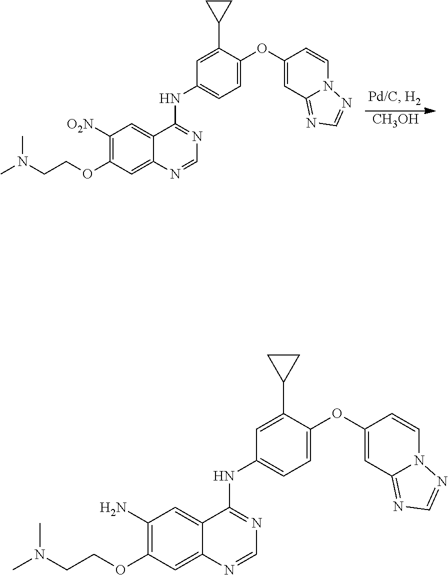

To a solution of N-(3-cyclopropyl-4-{[1,2,4]triazolo[1,5-a]pyridin-7-yloxy}phenyl)-7-[2-(dimethylamino)ethoxy]-6-nitroquinazolin-4-amine (600.0 mg, 1.14 mmol) in CH₃OH (10.0 mL) was added and Pd/C (200.0 mg, dry) at room temperature. The mixture was stirred at room temperature for 3 h under H₂. After the reaction was completed, the resulting mixture was filtered. The filtrate was concentrated under reduced pressure. The residue was purified by flash column chromatography with CH₂Cl₂/CH₃OH (7/3, v/v)) to afford N4-(4-([1,2,4]triazolo[1,5-a]pyridin-7-yloxy)-3-cyclopropylphenyl)-7-(2-(dimethylamino)ethoxy)quinazoline-4,6-diamine (200.0 mg, 35%) as a brown solid. LCMS (ESI, m/z): [M+H]⁺=497.2.

### Step 6: Synthesis of N-{4-[(3-cyclopropyl-4-{[1,2,4]triazolo[1,5-a]pyridin-7-yloxy}phenyl)amino]-7-[2-(dimethylamino)ethoxy]quinazolin-6-yl}but-2-ynamide (Compound 195)

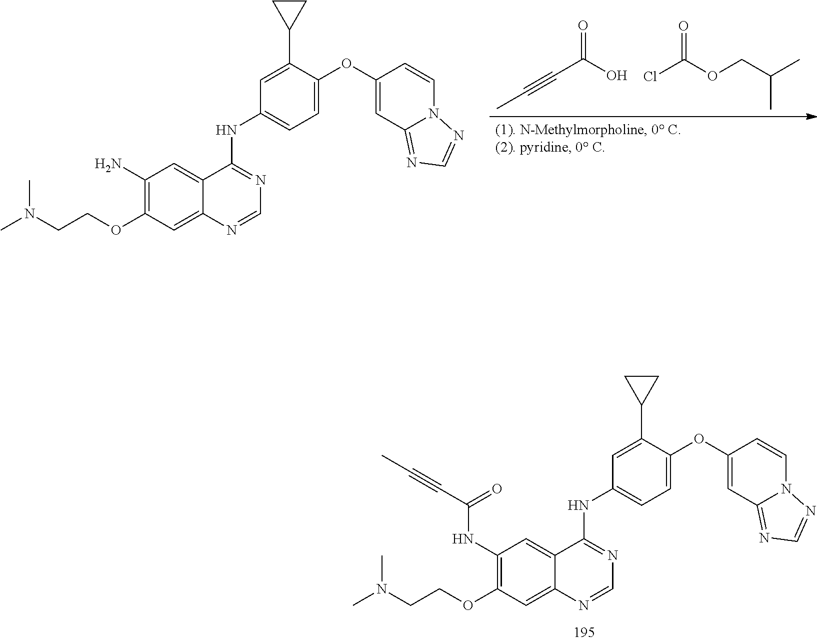

To a solution of 2-butynoic acid (182.8 mg, 2.17 mmol) in THF (10.0 mL) was added 2-methylpropyl carbonochloridate (297.0 mg, 2.17 mmol) at 0° C. The mixture was stirred at 0° C. for 5 min. Then N-Methylmorpholine (366.6 mg, 3.62 mmol) was added to the mixture at 0° C. The resulting mixture was stirred at 0° C. for additional 10 min. Then a solution of N4-(3-cyclopropyl-4-{[1,2,4]triazolo[1,5-a]pyridin-7-yloxy}phenyl)-7-[2-(dimethylamino)ethoxy]quinazoline-4,6-diamine (180.0 mg, 0.36 mmol) in pyridine (2.0 mL) was added dropwise to the mixture at 0° C. The mixture was stirred at 0° C. for additional 40 min. After the reaction was completed, the reaction mixture was diluted with water and extracted with ethyl acetate. The combined organic layer was washed with brine, dried over anhydrous Na₂SO₄ and filtered. The filtrate was concentrated under reduced pressure. The residue was purified by reverse phase flash column chromatography with CH₃CN/H₂O (1/1, v/v) and then purified by Prep-HPLC with the following conditions Column (XBridge Shield RP18 OBD Column, 30×150 mm, 5 um; Mobile Phase A: Water (10 mmol/L NH₄HCO₃), Mobile Phase B: ACN; Flow rate: 60 mL/min; Gradient: 25% B to 50% B in 8 min; Wave Length: 254 nm) to afford N-{4-[(3-cyclopropyl-4-{[1,2,4]triazolo[1,5-a]pyridin-7-yloxy}phenyl)amino]-7-[2-(dimethylamino)ethoxy]quinazolin-6-yl}but-2-ynamide (Compound 195) (21.8 mg, 10%) as a white solid. LCMS (ESI, m/z): [M+H]⁺=563.4. 1H NMR (400 MHz, DMSO-d₆): δ 10.22 (s, 1H), 9.68 (s, 1H), 8.92 (d, J=7.6 Hz, 1H), 8.69 (s, 1H), 8.52 (s, 1H), 8.38 (s, 1H), 7.86 (d, J=8.0 Hz, 1H), 7.43 (s, 1H), 7.34 (s, 1H), 7.18 (d, J=8.4 Hz, 1H), 7.06-7.03 (m, 1H), 6.83 (d, J=2.0 Hz, 1H), 4.30-4.27 (m, 2H), 2.72-2.69 (m, 2H), 2.27 (s, 6H), 2.08 (s, 3H), 1.96-1.90 (m, 1H), 0.92-0.88 (m, 2H), 0.69-0.64 (m, 2H).

### Example S85. N-(4-((4-([1,2,4]triazolo[1,5-a]pyridin-7-ylmethyl)-3-methylphenyl)amino)-7-(2-(dimethylamino)ethoxy)quinazolin-6-yl)but-2-ynamide (Compound 196)

### Step 1: Synthesis of N-(4-([1,2,4]triazolo[1,5-a]pyridin-7-ylmethyl)-3-methylphenyl)-7-(2-(dimethylamino)ethoxy)-6-nitroquinazolin-4-amine

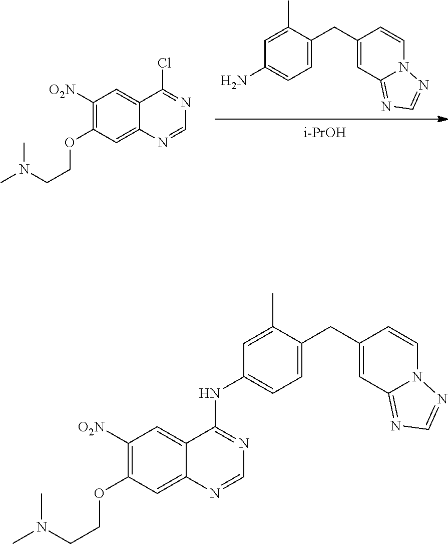 [SMILES: "i-PrOH" ; CN(C)CCOc1cc2ncnc(Cl)c2cc1[N+](=O)[O-] ; Cc1cc(N)ccc1Cc1ccn2ncnc2c1 ; Cc1cc(Nc2ncnc3cc(OCCN(C)C)c([N+](=O)[O-])cc23)ccc1Cc1ccn2ncnc2c1]

A mixture of 2-((4-chloro-6-nitroquinazolin-7-yl)oxy)-N,N-dimethylethan-1-amine (500.0 mg, crude) and 4-([1,2,4]triazolo[1,5-a]pyridin-7-ylmethyl)-3-methylaniline (426.0 mg, 1.79 mmol) in i-PrOH (10.0 mL) was stirred at 25° C. for 16 h. After the reaction was completed, the mixture was concentrated under reduced pressure. The residue was purified by flash column chromatography with CH₂Cl₂/MeOH (7/3, v/v) to afford N-(4-([1,2,4]triazolo[1,5-a]pyridin-7-ylmethyl)-3-methylphenyl)-7-(2-(dimethylamino)ethoxy)-6-nitroquinazolin-4-amine (800.0 mg, 89%) as a yellow solid. LCMS (ESI, m/z): =499.2.

### Step 2: Synthesis of N4-(4-([1,2,4]triazolo[1,5-a]pyridin-7-ylmethyl)-3-methylphenyl)-7-(2-(dimethylamino)ethoxy)quinazoline-4,6-diamine

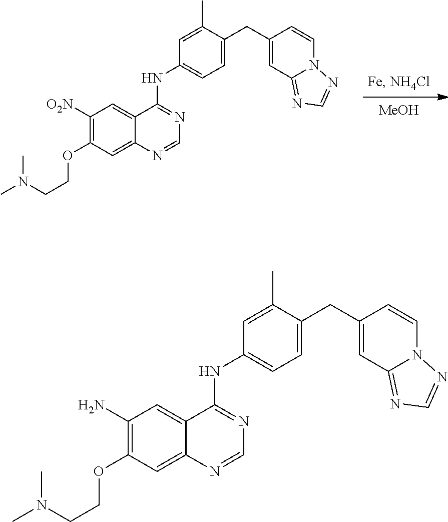

A mixture of N-(4-([1,2,4]triazolo[1,5-a]pyridin-7-ylmethyl)-3-methylphenyl)-7-(2-(dimethylamino)ethoxy)-6-nitroquinazolin-4-amine (800.0 mg, 1.60 mmol), Fe (898.8 mg, 16.05 mmol) and NH₄Cl (850.6 mg, 16.05 mmol) in CH₃OH (20.0 mL)/H₂O (4.0 mL) was heated at 80° C. for 1 h. After the reaction was completed, the resulting mixture was filtered. The filtrate was concentrated under reduced pressure. The residue was purified by reverse phase flash column chromatography with CH₃CN/H₂O (4/6, v/v) to afford N4-(4-([1,2,4]triazolo[1,5-a]pyridin-7-ylmethyl)-3-methylphenyl)-7-(2-(dimethylamino)ethoxy)quinazoline-4,6-diamine (300.0 mg, 40%) as a yellow solid. LCMS (ESI, m/z): [M+H]⁺=469.2.

### Step 3: Synthesis of N-(4-((4-([1,2,4]triazolo[1,5-a]pyridin-7-ylmethyl)-3-methylphenyl)amino)-7-(2-(dimethylamino)ethoxy)quinazolin-6-yl)but-2-ynamide (Compound 196)

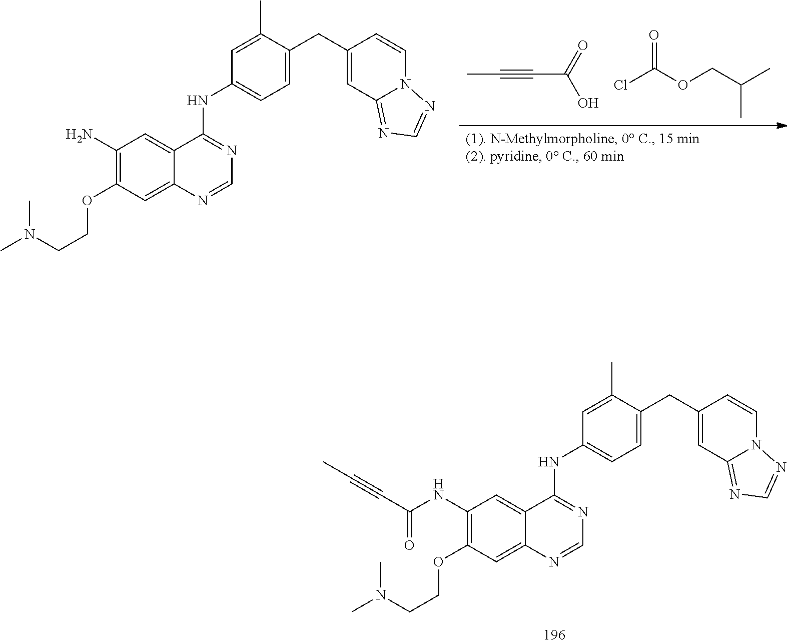

To a solution of but-2-ynoic acid (322.9 mg 3.84 mmol) in THF (10.0 mL) was added isobutyl carbonochloridate (524.6 mg, 3.84 mmol) at 0° C. The mixture was stirred at 0° C. for 5 min. Then N-Methylmorpholine (647.5 mg, 6.40 mmol) was added to the mixture at 0° C. The resulting mixture was stirred at 0° C. for additional 10 min. Then a solution of N4-(4-([1,2,4]triazolo[1,5-a]pyridin-7-ylmethyl)-3-methylphenyl)-7-(2-(dimethylamino)ethoxy)quinazoline-4,6-diamine (300.0 mg, 0.64 mmol) in pyridine (3.0 mL) was added dropwise to the mixture at 0° C. The mixture was stirred at 0° C. for additional 40 min. After the reaction was completed, the reaction was quenched with water and extracted with ethyl acetate. The combined organic layer was washed with brine, dried over anhydrous Na₂SO₄ and filtered. The filtrate was concentrated under reduced pressure. The residue was purified by reverse phase flash column chromatography with CH₃CN/H₂O (4/6, v/v) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 31% B to 36% B in 8 min; Wave Length: 254 nm) to afford N-(4-((4-([1,2,4]triazolo[1,5-a]pyridin-7-ylmethyl)-3-methylphenyl)amino)-7-(2-(dimethylamino)ethoxy)quinazolin-6-yl)but-2-ynamide (Compound 196) (31.6 mg, 9%) as a white solid. LCMS (ESI, m/z): [M+H]⁺=535.4. 1H NMR (400 MHz, DMSO-d₆): δ 10.22 (s, 1H), 9.64 (s, 1H), 8.86 (d, J=4.8 Hz, 1H), 8.69 (s, 1H), 8.49-8.44 (m, 2H), 7.68-7.62 (m, 2H), 7.52 (s, 1H), 7.32 (s, 1H), 7.23 (d, J=6.4 Hz, 1H), 7.03 (s, 1H), 4.32-4.26 (m, 2H), 4.12 (s, 2H), 2.78-2.72 (m, 2H), 2.34-2.26 (m, 9H), 2.08 (s, 3H).

### Example S86. N-(4-((4-([1,2,4]triazolo[1,5-a]pyridin-7-yloxy)-5-chloro-2-fluorophenyl)amino)-7-(2-(dimethylamino)ethoxy)quinazolin-6-yl)but-2-ynamide (Compound 197)

### Step 1: Synthesis of 7-(2-chloro-5-fluoro-4-nitrophenoxy)-[1,2,4]triazolo[1,5-a]pyridine

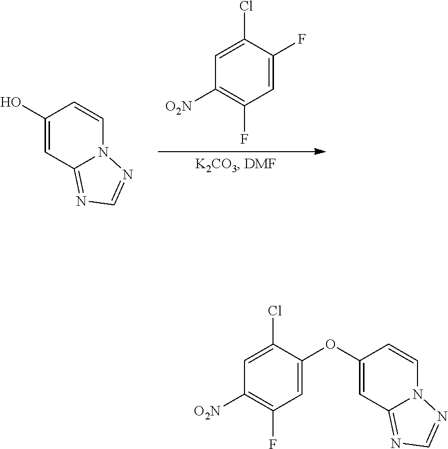

To a mixture of [1,2,4]triazolo[1,5-a]pyridin-7-ol (3.0 g, 22.20 mmol) and 1-chloro-2,4-difluoro-5-nitrobenzene (4.3 g, 22.20 mmol) in DMF (140.0 mL) was added K₂CO₃ (6.1 g, 44.40 mmol) at room temperature. The resulting mixture was stirred at 70° C. for 16 h. After the reaction was completed, the resulting mixture was diluted with H₂O and extracted with ethyl acetate. The combined organic layer was washed with brine, dried over anhydrous Na₂SO₄ and filtered. The filtrate was concentrated under reduced pressure. The residue was purified by flash column chromatography with petroleum ether/ethyl acetate (1/1, v/v) to afford 7-(2-chloro-5-fluoro-4-nitrophenoxy)-[1,2,4]triazolo[1,5-a]pyridine (2.5 g, 36%) as a brown yellow solid. LCMS (ESI, m/z): [M+H]⁺=309.0.

### Step 2: Synthesis of 4-([1,2,4]triazolo[1,5-a]pyridin-7-yloxy)-5-chloro-2-fluoroaniline

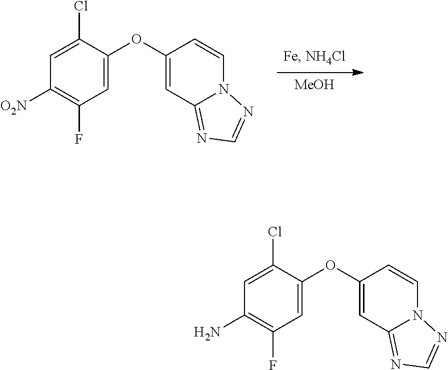

To a solution of 7-(2-chloro-5-fluoro-4-nitrophenoxy)-[1,2,4]triazolo[1,5-a]pyridine (2.5 g, 8.10 mmol) in CH₃OH (30.0 mL)/H₂O (6.0 mL) were added NH₄Cl (4.3 g, 81.00 mmol) and Fe (4.5 g, 81.00 mmol) at room temperature. The resulting mixture was stirred at 80° C. for 1 h. After the reaction was completed, the resulting mixture was filtered. The filtrate was concentrated under reduced pressure. The residue was purified by flash column chromatography with petroleum ether/ethyl acetate (1/1, v/v) to afford 4-([1,2,4]triazolo[1,5-a]pyridin-7-yloxy)-5-chloro-2-fluoroaniline (1.9 g, 84%) as a brown solid. LCMS (ESI, m/z): [M+H]⁺=279.0.

### Step 3: Synthesis of N-(4-([1,2,4]triazolo[1,5-a]pyridin-7-yloxy)-5-chloro-2-fluorophenyl)-7-(2-(dimethylamino)ethoxy)-6-nitroquinazolin-4-amine

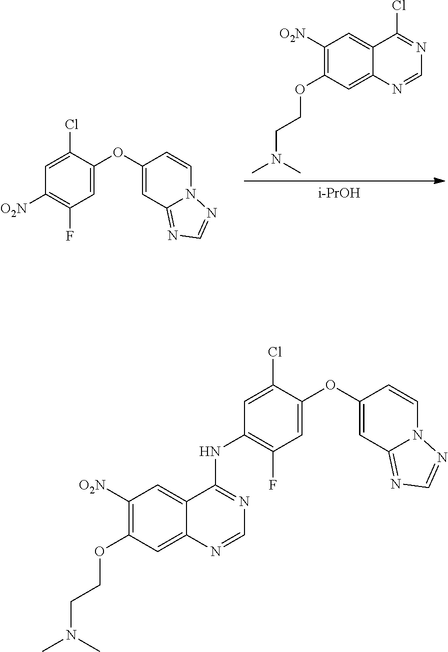 [SMILES: "i-PrOH" ; O=[N+]([O-])c1cc(Cl)c(Oc2ccn3ncnc3c2)cc1F ; CN(C)CCOc1cc2ncnc(Cl)c2cc1[N+](=O)[O-] ; CN(C)CCOc1cc2ncnc(Nc3cc(Cl)c(Oc4ccn5ncnc5c4)cc3F)c2cc1[N+](=O)[O-]]

To a solution of 5-chloro-2-fluoro-4-{[1,2,4]triazolo[1,5-a]pyridin-7-yloxy}aniline (798.3 mg, 2.86 mmol) in i-PrOH (16.0 mL) was added {2-[(4-chloro-6-nitroquinazolin-7-yl)oxy]ethyl}dimethylamine (850.0 mg, crude) at room temperature. The resulting mixture was stirred at room temperature for 16 h. After the reaction was completed, the resulting mixture was concentrated under reduced pressure. The residue was purified by flash column chromatography with CH₂Cl₂/CH₃OH (10/1, v/v) to afford N-(4-([1,2,4]triazolo[1,5-a]pyridin-7-yloxy)-5-chloro-2-fluorophenyl)-7-(2-(dimethylamino)ethoxy)-6-nitroquinazolin-4-amine (1.5 g, 97%) as a brown yellow solid. LCMS (ESI, m/z): [M+H]⁺=539.1.

### Step 4: Synthesis of N4-(4-([1,2,4]triazolo[1,5-a]pyridin-7-yloxy)-5-chloro-2-fluorophenyl)-7-(2-(dimethylamino)ethoxy)quinazoline-4,6-diamine

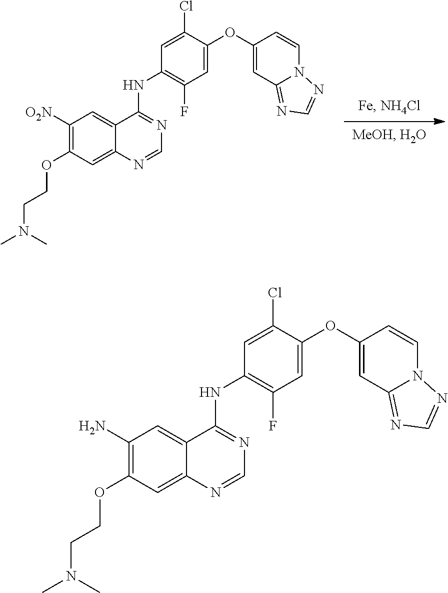

To a mixture of N-(5-chloro-2-fluoro-4-{[1,2,4]triazolo[1,5-a]pyridin-7-yloxy}phenyl)-7-[2-(dimethylamino)ethoxy]-6-nitroquinazolin-4-amine (1.5 g, 2.78 mmol) and NH₄Cl (1.5 g, 27.83 mmol) in CH₃OH (25.0 mL)/H₂O (5.0 mL) was added Fe (1.5 g, 27.83 mmol) at room temperature. The resulting mixture was stirred at 80° C. for 1 h. After the reaction was completed, the resulting mixture was filtered. The filtrate was concentrated under reduced pressure. The residue was purified by flash column chromatography with CH₂Cl₂/CH₃OH (5/1, v/v) to afford N4-(5-chloro-2-fluoro-4-{[1,2,4]triazolo[1,5-a]pyridin-7-yloxy}phenyl)-7-[2-(dimethylamino)ethoxy]quinazoline-4,6-diamine (1.0 g, 70%) as a yellow solid. LCMS (ESI, m/z): [M+H]⁺=509.1.

### Step 5: Synthesis of N-(4-((4-([1,2,4]triazolo[1,5-a]pyridin-7-yloxy)-5-chloro-2-fluorophenyl)amino)-7-(2-(dimethylamino)ethoxy)quinazolin-6-yl)but-2-ynamide (Compound 197)

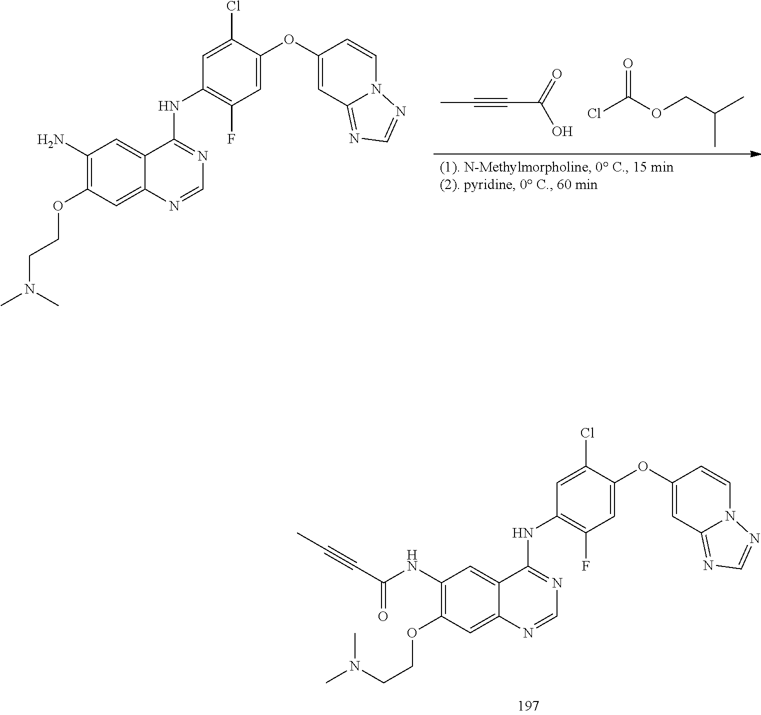

To a solution of but-2-ynoic acid (247.6 mg, 2.95 mmol) in THF (6.0 mL) was added 2-methylpropyl carbonochloridate (402.5 mg, 2.95 mmol) at 0° C. The resulting mixture was stirred at 0° C. for 5 min. Then N-Methylmorpholine (496.9 mg, 4.91 mmol) was added to the mixture at 0° C. The resulting mixture was stirred at 0° C. for additional 10 min. Then a solution of N4-(5-chloro-2-fluoro-4-{[1,2,4]triazolo[1,5-a]pyridin-7-yloxy}phenyl)-7-[2-(dimethylamino)ethoxy]quinazoline-4,6-diamine (500.0 mg, 0.98 mmol) in pyridine (6.0 mL) was added dropwise to the mixture at 0° C. The resulting mixture was stirred at 0° C. for additional 40 min. After the reaction was completed, the reaction mixture was diluted with water and extracted with ethyl acetate. The combined organic layer was washed with brine, dried over anhydrous Na₂SO₄ and filtered. The filtrate was concentrated under reduced pressure. The residue was purified by reverse phase flash column chromatography with acetonitrile/water (7/3, v/v) and then purified by Prep-HPLC with the following conditions (Column: XBridge Prep OBD C18 Column, 19×250 mm, 5 um; Mobile Phase A: Water (10 mmol/L NH₄HCO₃), Mobile Phase B: MeOH-HPLC; Flow rate: 25 mL/min; Gradient: 60% B to 80% B in 10 min; Wave Length: 254 nm) to afford N-{4-[(5-chloro-2-fluoro-4-{[1,2,4]triazolo[1,5-a]pyridin-7-yloxy}phenyl)amino]-7-[2-(dimethylamino)ethoxy]quinazolin-6-yl}but-2-ynamide (Compound 197) (38.1 mg, 6%) as a white solid. LCMS (ESI, m/z): [M+H]⁺=575.2. 1H NMR (400 MHz, DMSO-d₆): δ 10.23 (s, 1H), 9.86 (s, 1H), 9.00 (d, J=7.2 Hz, 1H), 8.73 (s, 1H), 8.48-8.45 (m, 2H), 7.92-7.85 (m, 1H), 7.64-7.58 (m, 1H), 7.38 (s, 1H), 7.17-7.13 (m, 2H), 4.31-4.25 (m, 2H), 2.74-2.65 (m, 2H), 2.28 (s, 6H), 2.08 (s, 3H).

### Example S87. N-(4-((4-([1,2,4]triazolo[1,5-a]pyridin-7-yloxy)-3-fluorophenyl)amino)-7-(2-(dimethylamino)ethoxy)quinazolin-6-yl)but-2-ynamide (Compound 198)

### Step 1: Synthesis of 7-(2-fluoro-4-nitrophenoxy)-[1,2,4]triazolo[1,5-a]pyridine

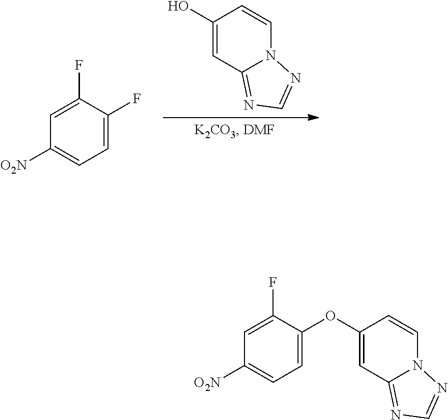

To a solution of 1,2-difluoro-4-nitrobenzene (1.0 g, 6.28 mmol) in DMF (20.0 mL) were added [1,2,4]triazolo[1,5-a]pyridin-7-ol (0.9 g, 6.28 mmol) and K₂CO₃ (1.7 g, 12.57 mmol) at room temperature. The resulting mixture was stirred at 50° C. for 16 h. After the reaction was completed, the resulting mixture was diluted with water and extracted with ethyl acetate. The combined organic layer was washed with brine, dried over anhydrous Na2SO4 and filtered. The filtrate was concentrated under reduced pressure to afford 7-(2-fluoro-4-nitrophenoxy)-[1,2,4]triazolo[1,5-a]pyridine (2.0 g, 81%) as a white solid. LCMS (ESI, m/z): [M+H]⁺=275.1.

### Step 2: Synthesis of 4-([1,2,4]triazolo[1,5-a]pyridin-7-yloxy)-3-fluoroaniline

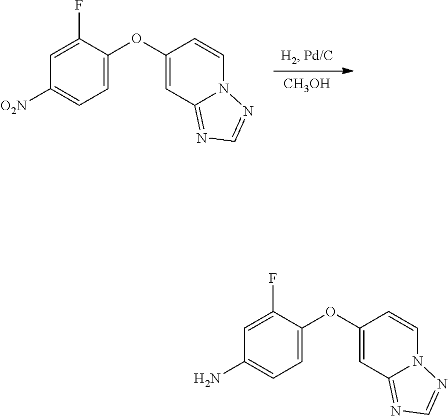 [SMILES: O=[N+]([O-])c1ccc(Oc2ccn3ncnc3c2)c(F)c1 ; "CH3OH" ; "H2, Pd/C" ; Nc1ccc(Oc2ccn3ncnc3c2)c(F)c1]

To a solution of 7-(2-fluoro-4-nitrophenoxy)-[1,2,4]triazolo[1,5-a]pyridine (2.0 g, 6.89 mmol) in methanol (30.0 mL) was added Pd/C (220.0 mg, dry) at room temperature. The resulting mixture was stirred at room temperature for 3 h under H₂. After the reaction was completed, the resulting mixture was filtered. The filtrate was concentrated under reduced pressure to afford 4-([1,2,4]triazolo[1,5-a]pyridin-7-yloxy)-3-fluoroaniline (1.0 g, crude) as a light yellow solid. LCMS (ESI, m/z): [M+H]⁺=245.1.

### Step 3: Synthesis of N-(4-([1,2,4]triazolo[1,5-a]pyridin-7-yloxy)-3-fluorophenyl)-7-(2-(dimethylamino)ethoxy)-6-nitroquinazolin-4-amine

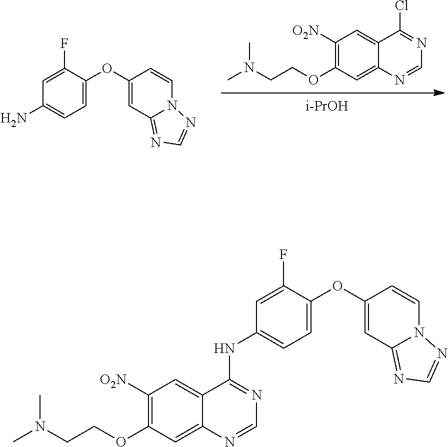 [SMILES: "i-PrOH" ; Nc1ccc(Oc2ccn3ncnc3c2)c(F)c1 ; CN(C)CCOc1cc2ncnc(Cl)c2cc1[N+](=O)[O-] ; CN(C)CCOc1cc2ncnc(Nc3ccc(Oc4ccn5ncnc5c4)c(F)c3)c2cc1[N+](=O)[O-]]

To a solution of 4-([1,2,4]triazolo[1,5-a]pyridin-7-yloxy)-3-fluoroaniline (1.0 g, 4.27 mmol) in isopropyl alcohol (20.0 mL) was added {2-[(4-chloro-6-nitroquinazolin-7-yl)oxy]ethyl}dimethylamine (2.5 g, 8.54 mmol) at room temperature. The resulting mixture was stirred at room temperature for 1 h. After the reaction was completed, the resulting mixture was concentrated under reduced pressure. The residue was purified by flash column chromatography with CH₂Cl₂/CH₃OH (4/1, v/v) to afford N-(4-([1,2,4]triazolo[1,5-a]pyridin-7-yloxy)-3-fluorophenyl)-7-(2-(dimethylamino)ethoxy)-6-nitroquinazolin-4-amine (1.5 g, 69%) as a yellow solid. LCMS (ESI, m/z): [M+H]⁺=505.0.

### Step 4: Synthesis of N4-(4-([1,2,4]triazolo[1,5-a]pyridin-7-yloxy)-3-fluorophenyl)-7-(2-(dimethylamino)ethoxy)quinazoline-4,6-diamine

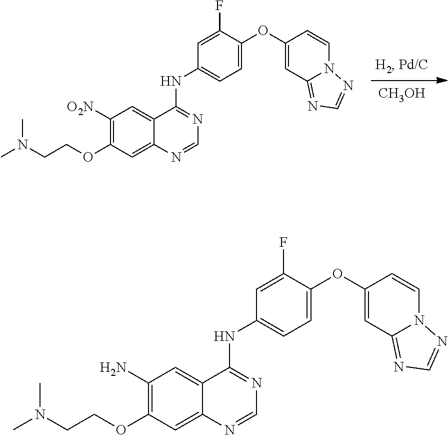

To a solution of 7-[2-(dimethylamino)ethoxy]-N-(3-fluoro-4-{[1,2,4]triazolo[1,5-a]pyridin-7-yloxy}phenyl)-6-nitroquinazolin-4-amine (1.5 g, 2.97 mmol) in CH₃OH (22.5 mL) was added Pd/C (0.1 g, dry) at room temperature. The resulting mixture was stirred at room temperature for 3 h under H₂. After the reaction was completed, the resulting mixture was filtered. The filtrate was concentrated under reduced pressure. The residue was purified by flash column chromatography with CH₂Cl₂/CH₃OH (5/1, v/v) to afford N4-(4-([1,2,4]triazolo[1,5-a]pyridin-7-yloxy)-3-fluorophenyl)-7-(2-(dimethylamino)ethoxy)quinazoline-4,6-diamine (1.0 g, 64%) as a yellow solid. LCMS (ESI, m/z): [M+H]⁺=475.0.

### Step 5: Synthesis of N-(4-((4-([1,2,4]triazolo[1,5-a]pyridin-7-yloxy)-3-fluorophenyl)amino)-7-(2-(dimethylamino)ethoxy)quinazolin-6-yl)but-2-ynamide (Compound 198)

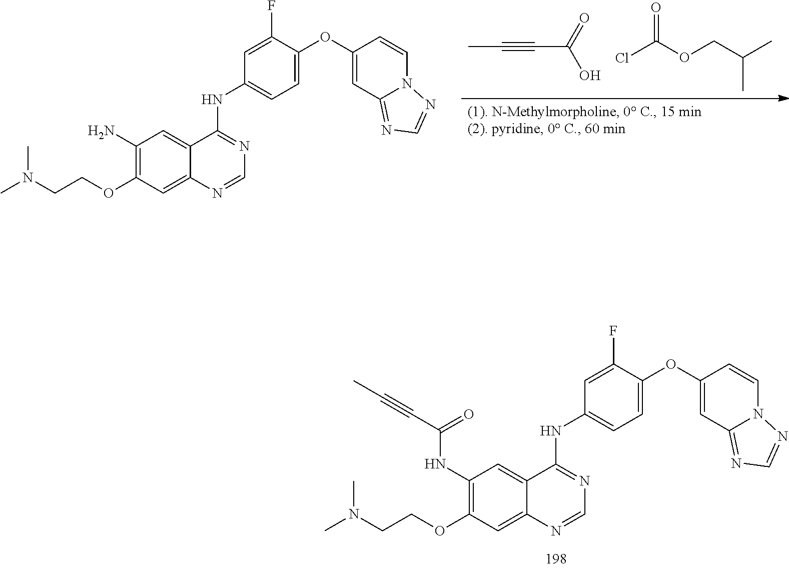

A mixture of 2-butynoic acid (318.9 mg, 3.79 mmol) and 2-methylpropyl carbonochloridate (518.1 mg, 3.79 mmol) in THF (10.0 mL) was stirred at 0° C. for 5 min. Then N-Methylmorpholine (639.5 mg, 6.32 mmol) was added to the mixture at 0° C. The resulting mixture was stirred at 0° C. for additional 10 min. Then a solution of 7-[2-(dimethylamino)ethoxy]-N4-(3-fluoro-4-{[1,2,4]triazolo[1,5-a]pyridin-7-yloxy}phenyl)quinazoline-4,6-diamine (300.0 mg, 0.63 mmol) in pyridine (3.0 mL) was added dropwise to the mixture at 0° C. The resulting mixture was stirred at 0° C. for additional 40 min. After the reaction was completed, the resulting mixture was quenched with sat. NH₄Cl (aq.) and extracted with ethyl acetate. The combined organic layer was washed with brine, dried over anhydrous Na₂SO₄ and filtered. The filtrate was concentrated under reduced pressure. The residue was purified by reverse phase flash column chromatography with acetonitrile/water (64/36, v/v) and then purified by Prep-HPLC with the following conditions (Column: XBridge Shield RP18 OBD Column, 19×250 mm, 10 um; Mobile Phase A: Water (0.05% FA), Mobile Phase B: MeOH-HPLC; Flow rate: 25 mL/min; Gradient: 25% B to 30% B in 12 min; Wave Length: 254 nm) to afford N-(4-((4-([1,2,4]triazolo[1,5-a]pyridin-7-yloxy)-3-fluorophenyl)amino)-7-(2-(dimethylamino)ethoxy)quinazolin-6-yl)but-2-ynamide (Compound 198) (22.2 mg, 6%) as a white solid. LCMS (ESI, m/z): [M+H]+=541.2. 1H NMR (400 MHz, DMSO-d₆): δ 10.29 (s, 1H), 9.94 (s, 1H), 8.98 (d, J=7.2 Hz, 1H), 8.75 (s, 1H), 8.69 (s, 1H), 8.43 (s, 1H), 8.23-8.17 (m, 1H), 7.78 (d, J=9.2 Hz, 1H), 7.49-7.44 (m, 1H), 7.39 (s, 1H), 7.12-7.07 (m, 1H), 7.04 (s, 1H), 4.33-4.30 (m, 2H), 2.77-2.69 (m, 2H), 2.31 (s, 6H), 2.09 (s, 3H).

### Example S88. N-(4-((4-([1,2,4]triazolo[1,5-a]pyridin-7-yloxy)-3-methylphenyl)amino)pyrido[3,4-d]pyrimidin-6-yl)but-2-ynamide (Compound 199)

### Step 1: Synthesis of 4,6-dichloropyrido[3,4-d]pyrimidine

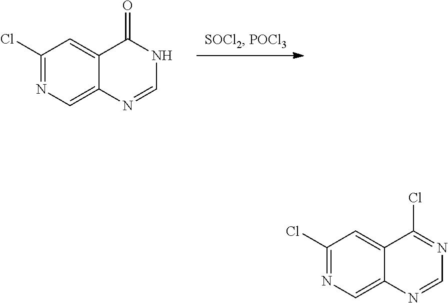

To a solution of 6-chloro-3H-pyrido[3,4-d]pyrimidin-4-one (1.5 g, 8.26 mmol) in SOCl₂ (15.0 mL) were added POCl₃ (13.0 mL) and DMF (0.1 mL) at room temperature. The resulting mixture was stirred at 85° C. for 1 h. After the reaction was completed, the mixture was evaporated in vacuo to afford 4,6-dichloropyrido[3,4-d]pyrimidine (1.6 g, crude) as a yellow solid. LCMS (ESI, m/z): [M+H]⁺=200.2.

### Step 2: Synthesis of N-(4-([1,2,4]triazolo[1,5-a]pyridin-7-yloxy)-3-methylphenyl)-6-chloropyrido[3,4-d]pyrimidin-4-amine

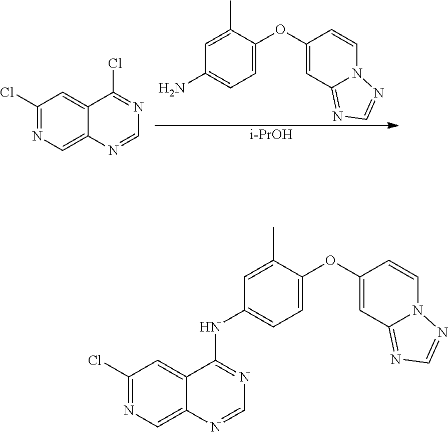 [SMILES: "i-PrOH" ; Clc1cc2c(Cl)ncnc2cn1 ; Cc1cc(N)ccc1Oc1ccn2ncnc2c1 ; Cc1cc(Nc2ncnc3cnc(Cl)cc23)ccc1Oc1ccn2ncnc2c1]

A mixture of 4,6-dichloropyrido[3,4-d]pyrimidine (1.6 g, crude) and 3-methyl-4-{[1,2,4]triazolo[1,5-a]pyridin-7-yloxy}aniline (2.0 g, 8.26 mmol) in i-PrOH (30.0 mL) was stirred at room temperature for 3 h. After the reaction was completed, the mixture was concentrated under reduced pressure. The residue was purified by flash column chromatography with CH₂Cl₂/MeOH (7/3, v/v) to afford N-(4-([1,2,4]triazolo[1,5-a]pyridin-7-yloxy)-3-methylphenyl)-6-chloropyrido[3,4-d]pyrimidin-4-amine (2.0 g, 60%) as a brown solid. LCMS (ESI, m/z): [M+H]⁺=404.1.

### Step 3: Synthesis of tert-butyl (4-((4-([1,2,4]triazolo[1,5-a]pyridin-7-yloxy)-3-methylphenyl)amino)pyrido[3,4-d]pyrimidin-6-yl)carbamate

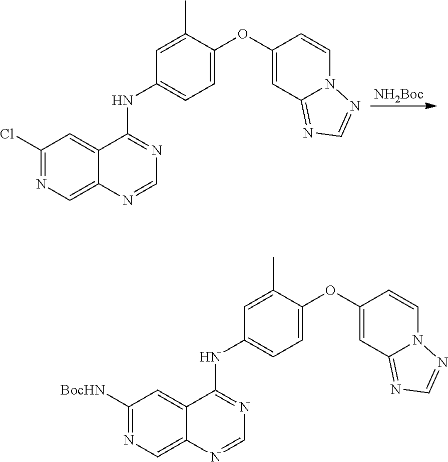 [SMILES: Cc1cc(Nc2ncnc3cnc(Cl)cc23)ccc1Oc1ccn2ncnc2c1 ; Cc1cc(Nc2ncnc3cnc(NC(=O)OC(C)(C)C)cc23)ccc1Oc1ccn2ncnc2c1 ; "NH2Boc"]

To a mixture of 6-chloro-N-(3-methyl-4-{[1,2,4]triazolo[1,5-a]pyridin-7-yloxy}phenyl)pyrido[3,4-d]pyrimidin-4-amine (1.4 g, 3.46 mmol), BocNH2 (1.2 g, 10.40 mmol) and Cs₂CO₃ (3.3 g, 10.40 mmol) in dioxane (15.0 mL) were added Pd(OAc)₂ (155.6 mg, 0.69 mmol) and XPhos (661.0 mg, 1.38 mmol) at room temperature under N₂. The mixture was stirred at 100° C. for 16 h under N₂. After the reaction was completed, the mixture was diluted with water and extracted with ethyl acetate. The combined organic layer was washed with brine, dried over anhydrous Na2SO4 and filtered. The filtrate was concentrated under reduced pressure. The residue was purified by flash column chromatography with CH₂Cl₂/MeOH (9/1, v/v) to afford tert-butyl ([1,2,4]triazolo[1,5-a]pyridin-7-yloxy)-3-methylphenyl)amino)pyrido[3,4-d]pyrimidin-6-yl)carbamate (1.5 g, 89%) as a brown solid. LCMS (ESI, m/z): [M+H]⁺=485.2.

### Step 4: Synthesis of N4-(4-([1,2,4]triazolo[1,5-a]pyridin-7-yloxy)-3-methylphenyl)pyrido[3,4-d]pyrimidine-4,6-diamine

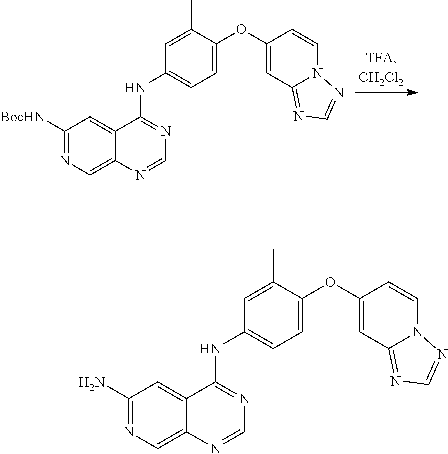 [SMILES: Cc1cc(Nc2ncnc3cnc(NC(=O)OC(C)(C)C)cc23)ccc1Oc1ccn2ncnc2c1 ; Cc1cc(Nc2ncnc3cnc(N)cc23)ccc1Oc1ccn2ncnc2c1]

A mixture of tert-butyl N-{4-[(3-methyl-4-{[1,2,4]triazolo[1,5-a]pyridin-7-yloxy}phenyl)amino]pyrido[3,4-d]pyrimidin-6-yl}carbamate (1.5 g, 3.09 mmol) and TFA (10.0 mL) in CH₂Cl₂ (20.0 mL) was stirred at room temperature for 1 h. After the reaction was completed, the mixture was concentrated under reduced pressure. The residue was purified by flash column chromatography with CH₂Cl₂/MeOH (9/1, v/v) to afford N4-(3-methyl-4-{[1,2,4]triazolo[1,5-a]pyridin-7-yloxy}phenyl)pyrido[3,4-d]pyrimidine-4,6-diamine (1.0 g, 84%) as a brown solid. LCMS (ESI, m/z): [M+H]⁺=385.1

### Step 5: Synthesis of N-(4-((4-([1,2,4]triazolo[1,5-a]pyridin-7-yloxy)-3-methylphenyl)amino)pyrido[3,4-d]pyrimidin-6-yl)but-2-ynamide (Compound 199)

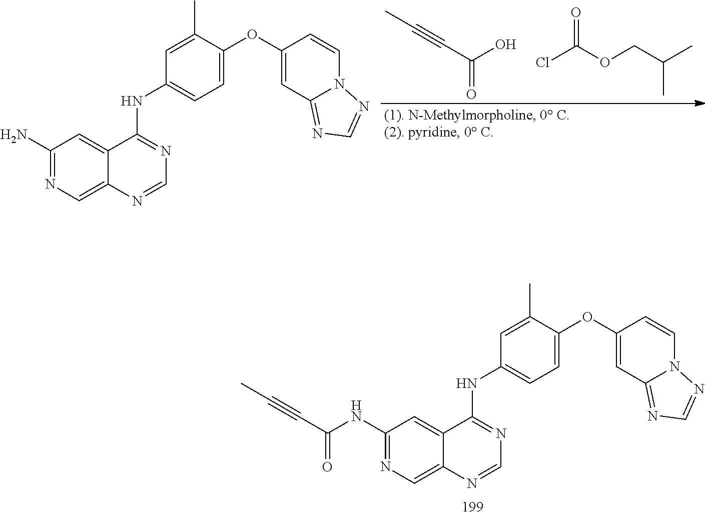

To a solution of 2-butynoic acid (393.6 mg, 4.68 mmol) in THF (10.0 mL) was added 2-methylpropyl carbonochloridate (639.5 mg, 4.68 mmol) at 0° C. The mixture was stirred at 0° C. for 5 min. Then N-Methylmorpholine (789.3 mg, 7.80 mmol) was added to the mixture at 0° C. The resulting mixture was stirred at 0° C. for additional 10 min. Then a solution of N4-(3-methyl-4-{[1,2,4]triazolo[1,5-a]pyridin-7-yloxy}phenyl)pyrido[3,4-d]pyrimidine-4,6-diamine (300.0 mg, 0.78 mmol) in pyridine (3.0 mL) was added dropwise to the mixture at 0° C. The mixture was stirred at 0° C. for additional 40 min. After the reaction was completed, the reaction mixture was diluted with water and extracted with ethyl acetate. The combined organic layer was washed with brine, dried over anhydrous Na₂SO₄ and filtered. The filtrate was concentrated under reduced pressure. The residue was purified by reverse phase flash column chromatography with CH₃CN/H₂O (4/6, v/v) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 31% B to 36% B in 8 min; Wave Length: 254 nm) to afford N-{4-[(3-methyl-4-{[1,2,4]triazolo[1,5-a]pyridin-7-yloxy}phenyl)amino]pyrido[3,4-d]pyrimidin-6-yl}but-2-ynamide (Compound 199) (24.1 mg, 6%) as a yellow solid. LCMS (ESI, m/z): [M+H]⁺=451.2. 1H NMR (400 MHz, DMSO-d6): δ 11.38 (s, 1H), 10.28 (s, 1H), 9.01 (s, 1H), 8.94 (d, J=7.6 Hz, 1H), 8.84 (s, 1H), 8.63 (s, 1H), 8.38 (s, 1H), 7.84-7.80 (m, 2H), 7.22 (d, J=8.8 Hz, 1H), 7.05-7.02 (m, 1H), 6.82 (d, J=2.0 Hz, 1H), 2.21 (s, 3H), 2.09 (s, 3H).

### Example S89. N-(4-((4-([1,2,4]triazolo[1,5-a]pyridin-7-yloxy)-3-chlorophenyl)amino)-7-(2-(dimethylamino)ethoxy)quinazolin-6-yl)but-2-ynamide (Compound 200)

### Step 1: Synthesis of 7-(2-chloro-4-nitrophenoxy)-[1,2,4]triazolo[1,5-a]pyridine

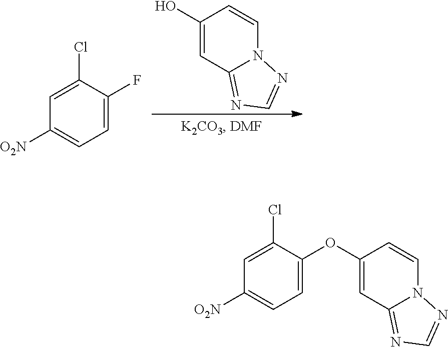 [SMILES: O=[N+]([O-])c1ccc(F)c(Cl)c1 ; Oc1ccn2ncnc2c1 ; O=[N+]([O-])c1ccc(Oc2ccn3ncnc3c2)c(Cl)c1]

To a solution of 2-chloro-1-fluoro-4-nitrobenzene (500.0 mg, 2.84 mmol) in DMF (10.0 mL) was added [1,2,4]triazolo[1,5-a]pyridin-7-ol (423.3 mg, 3.13 mmol) and K2CO3 (787.3 mg, 5.69 mmol) at room temperature. The mixture was stirred at 50° C. for 16 h. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH2Cl2/CH3OH (7/3, v/v) to afford 7-(2-chloro-4-nitrophenoxy)-[1,2,4]triazolo[1,5-a]pyridine (800.0 mg, 97%) as a brown solid. LCMS (ESI, m/z): [M+H]⁺=291.0.

### Step 2: Synthesis of 4-([1,2,4]triazolo[1,5-a]pyridin-7-yloxy)-3-chloroaniline

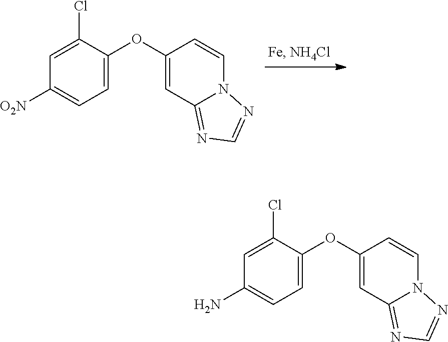 [SMILES: O=[N+]([O-])c1ccc(Oc2ccn3ncnc3c2)c(Cl)c1 ; Nc1ccc(Oc2ccn3ncnc3c2)c(Cl)c1]

A mixture of 7-(2-chloro-4-nitrophenoxy)-[1,2,4]triazolo[1,5-a]pyridine (800.0 mg, 2.75 mmol), Fe (1.5 g, 27.52 mmol) and NH₄Cl (1.4 g, 27.52 mmol) in CH₃OH (20.0 mL)/H₂O (2.0 mL) was heated at 80° C. for 1 h. After the reaction was completed, the resulting mixture was filtered. The filtrate was concentrated under reduced pressure. The residue was purified by flash column chromatography with CH₂Cl₂/CH₃OH (9/1, v/v) to afford 4-([1,2,4]triazolo[1,5-a]pyridin-7-yloxy)-3-chloroaniline (600.0 mg, 83%) as a yellow solid. LCMS (ESI, m/z): [M+H]⁺=261.0.

### Step 3: Synthesis of N-(4-([1,2,4]triazolo[1,5-a]pyridin-7-yloxy)-3-chlorophenyl)-7-(2-(dimethylamino)ethoxy)-6-nitroquinazolin-4-amine

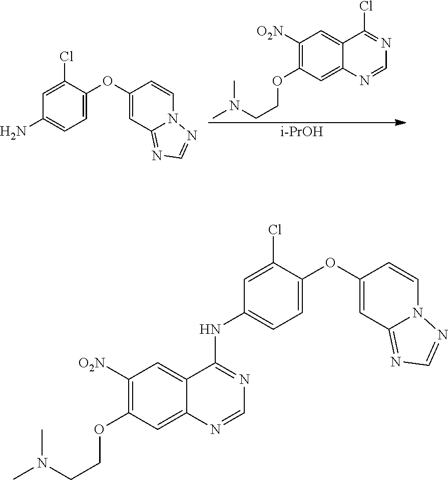

A mixture of 3-chloro-4-{[1,2,4]triazolo[1,5-a]pyridin-7-yloxy}aniline (500.0 mg, 1.91 mmol) and {2-[(4-chloro-6-nitroquinazolin-7-yl)oxy]ethyl}dimethylamine (569.1 mg, crude) in i-PrOH (10.0 mL) was stirred at room temperature for 2 h. After the reaction was completed, the mixture was concentrated under reduced pressure. The residue was purified by flash column chromatography with CH₂Cl₂/MeOH (7/3, v/v) to afford N-(4-([1,2,4]triazolo[1,5-a]pyridin-7-yloxy)-3-chlorophenyl)-7-(2-(dimethylamino)ethoxy)-6-nitroquinazolin-4-amine (1.0 g, 90%) as a yellow solid. LCMS (ESI, m/z): [M+H]⁺=521.1.

### Step 4: Synthesis of N4-(4-([1,2,4]triazolo[1,5-a]pyridin-7-yloxy)-3-chlorophenyl)-7-(2-(dimethylamino)ethoxy)quinazoline-4,6-diamine

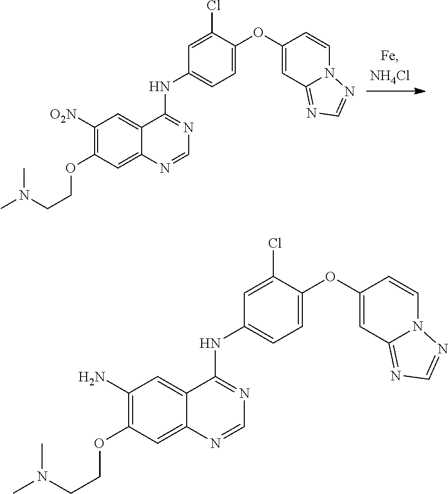 [SMILES: CN(C)CCOc1cc2ncnc(Nc3ccc(Oc4ccn5ncnc5c4)c(Cl)c3)c2cc1[N+](=O)[O-] ; CN(C)CCOc1cc2ncnc(Nc3ccc(Oc4ccn5ncnc5c4)c(Cl)c3)c2cc1N]

A mixture of N-(3-chloro-4-{[1,2,4]triazolo[1,5-a]pyridin-7-yloxy}phenyl)-7-[2-(dimethylamino)ethoxy]-6-nitroquinazolin-4-amine (800.0 mg, 1.53 mmol) and Fe (857.6 mg, 15.36 mmol), NH₄Cl (821.4 mg, 15.36 mmol) in CH₃OH (20.0 mL)/H₂O (2.0 mL) was heated at 80° C. for 1 h. After the reaction was completed, the resulting mixture was filtered. The filtrate was concentrated under reduced pressure. The residue was purified by flash column chromatography with CH₂Cl₂/CH₃OH (7/3, v/v) to afford N4-(4-([1,2,4]triazolo[1,5-a]pyridin-7-yloxy)-3-chlorophenyl)-7-(2-(dimethylamino)ethoxy)quinazoline-4,6-diamine (350.0 mg, 44%) as a yellow solid. LCMS (ESI, m/z): [M+H]⁺=491.2.

### Synthesis of N-(4-((4-([1,2,4]triazolo[1,5-a]pyridin-7-yloxy)-3-chlorophenyl)amino)-7-(2-(dimethylamino)ethoxy)quinazolin-6-yl)but-2-ynamide (Compound 200)

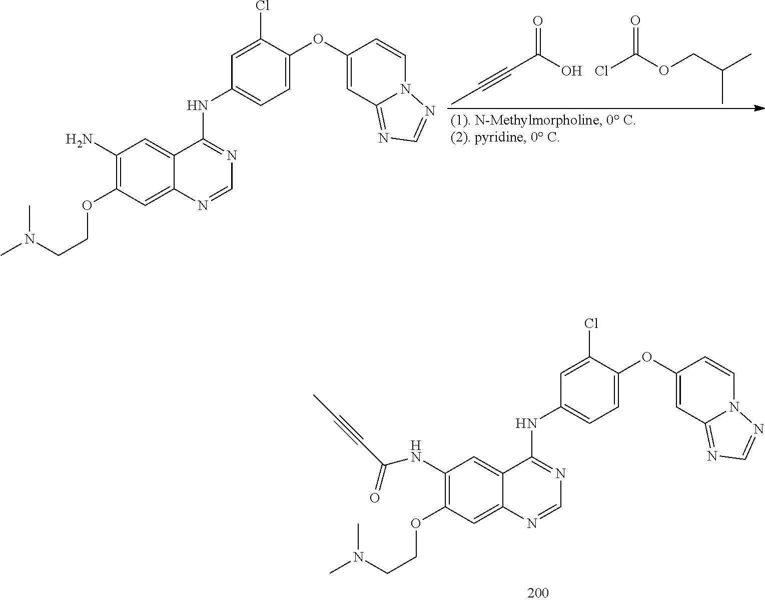

To a solution of 2-butynoic acid (308.2 mg, 3.66 mmol) in THF (10.0 mL) was added 2-methylpropyl carbonochloridate (500.7 mg, 3.66 mmol) at 0° C. The mixture was stirred at 0° C. for 5 min. To the above mixture was added N-Methylmorpholine (618.0 mg, 6.11 mmol) at 0° C. The resulting mixture was stirred at 0° C. for additional 10 min. Then a solution of N4-(3-chloro-4-{[1,2,4]triazolo[1,5-a]pyridin-7-yloxy}phenyl)-7-[2-(dimethylamino)ethoxy]quinazoline-4,6-diamine (300.0 mg, 0.61 mmol) in pyridine (3.0 mL) was added dropwise to the mixture at 0° C. The mixture was stirred at 0° C. for additional 40 min. After the reaction was completed, the reaction mixture was diluted with water and extracted with ethyl acetate. The combined organic layer was washed with brine, dried over anhydrous Na₂SO₄ and filtered. The filtrate was concentrated under reduced pressure. The residue was purified by reverse phase flash column chromatography with CH3CN/H2O (1/1, v/v) and then purified by Prep-HPLC with the following conditions (Column: XBridge Prep OBD C18 Column, 30×150 mm, 5 μm; Mobile Phase A: Water (10 mmol/L NH4HCO3), Mobile Phase B: ACN; Flow rate: 60 mL/min; Gradient: 30% B to 42% B in 8 min; Wave Length: 254 nm) to afford N-(4-((4-([1,2,4]triazolo[1,5-a]pyridin-7-yloxy)-3-chlorophenyl)amino)-7-(2-(dimethylamino)ethoxy)quinazolin-6-yl)but-2-ynamide (Compound 200) (43.7 mg, 12%) as a white solid. LCMS (ESI, m/z): [M+H]⁺=557.3. 1H NMR (400 MHz, CD3OD): δ 8.91 (s, 1H), 8.76 (d, J=7.6 Hz, 1H), 8.53 (s, 1H), 8.31 (s, 1H), 8.18 (d, J=2.0 Hz, 1H), 7.86-7.83 (m, 1H), 7.37 (d, J=8.8 Hz, 1H), 7.24 (s, 1H), 7.10-7.08 (m, 1H), 6.89 (d, J=2.4 Hz, 1H), 4.35-4.33 (m, 2H), 2.91-2.89 (m, 2H), 2.45 (s, 6H), 2.11 (s, 3H).

### Example S90. N-{7-[2-(dimethylamino)ethoxy]-4-([3-methyl-4-[(1-methyl-6-oxopyridazin-4-yl)oxy]phenyl}amino)quinazolin-6-yl]but-2-ynamide (Compound 201)

### Step 1: Synthesis of 4-chloro-5-methoxy-2-methylpyridazin-3(2H)-one

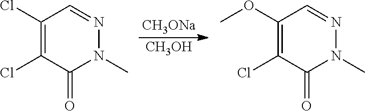

To a solution of 4,5-dichloro-2-methylpyridazin-3(2H)-one (2.0 g, 11.17 mmol) in CH3OH (40.0 mL) was added CH3ONa (724.3 mg, 13.41 mmol) at room temperature. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure to afford 4-chloro-5-methoxy-2-methylpyridazin-3(2H)-one (1.8 g, crude) as a yellow solid. LCMS (ESI, m/z): [M+H]⁺=175.0.

### Step 2: Synthesis of 5-methoxy-2-methylpyridazin-3(2H)-one

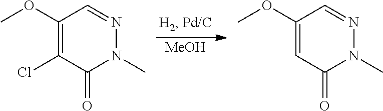

To a solution of 4-chloro-5-methoxy-2-methylpyridazin-3(2H)-one (1.5 g, 8.59 mmol) in CH3OH (10.0 mL) was added Pd/C (301.7 mg, dry) at room temperature. The resulting mixture was stirred at room temperature for 16 h under H2. After the reaction was completed, the resulting mixture was filtered. The filtrate was concentrated under reduced pressure to afford 5-methoxy-2-methylpyridazin-3(2H)-one (1.2 g, crude) as a yellow solid. LCMS (ESI, m/z): [M+H]+=141.1.

### Step 3: Synthesis of 5-hydroxy-2-methylpyridazin-3(2H)-one

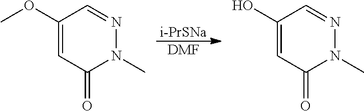

To a solution of 5-methoxy-2-methylpyridazin-3(2H)-one (680.0 mg, crude) in DMF (10.0 mL) was added sodium 2-methyl-2-propanethiolate technical grade (1.6 g, 14.56 mmol) at room temperature. The resulting mixture was stirred at 135° C. for 30 min. After the reaction was completed, the resulting mixture was concentrated under reduced pressure. The residue was purified by reverse phase flash chromatography with CH3CN/H2O (1/99, v/v) to afford 5-hydroxy-2-methylpyridazin-3(2H)-one (600.0 mg, 98%) as a yellow solid. LCMS (ESI, m/z): [M+H]+=127.0.

### Step 4: Synthesis of 2-methyl-5-(2-methyl-4-nitrophenoxy)pyridazin-3(2H)-one

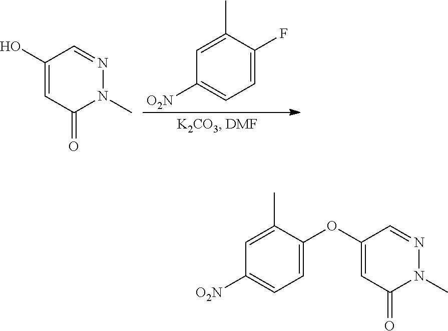

To a solution of 5-hydroxy-2-methylpyridazin-3(2H)-one (600.0 mg, 1.16 mmol) in DMF (10.0 mL) was added 1-fluoro-2-methyl-4-nitrobenzene (738.4 mg, 4.76 mmol) and K2CO3 (2.0 g, 14.28 mmol) at room temperature under N2. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/99, v/v) to afford 2-methyl-5-(2-methyl-4-nitrophenoxy)pyridazin-3(2H)-one (230.0 mg, 38%) as a brown solid. LCMS (ESI, m/z): [M+H]+=262.1.

### Step 5: Synthesis of 5-(4-amino-2-methylphenoxy)-2-methylpyridazin-3(2H)-one

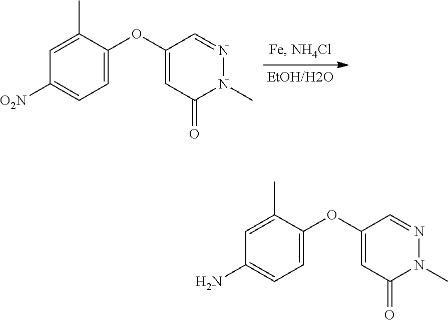 [SMILES: Cc1cc([N+](=O)[O-])ccc1Oc1cnn(C)c(=O)c1 ; Cc1cc(N)ccc1Oc1cnn(C)c(=O)c1]

To a solution of 2-methyl-5-(2-methyl-4-nitrophenoxy)pyridazin-3(2H)-one (230.0 mg, 0.88 mmol) in EtOH/H2O (5.0/5.0 mL) was added Fe (245.8 mg, 4.40 mmol) and NH4Cl (235.5 mg, 4.40 mmol) at room temperature. The resulting mixture was stirred at 80° C. for 4 h. After the reaction was completed, the resulting mixture was filtered. The filtrate was concentrated under reduced pressure to afford 5-(4-amino-2-methylphenoxy)-2-methylpyridazin-3(2H)-one (200.0 mg, crude) as a brown oil. LCMS (ESI, m/z): [M+H]+=232.1.

### Step 6: Synthesis of 5-[4-({7-[2-(dimethylamino)ethoxy]-6-nitroquinazolin-4-yl}amino)-2-methylphenoxy]-2-methylpyridazin-3-one

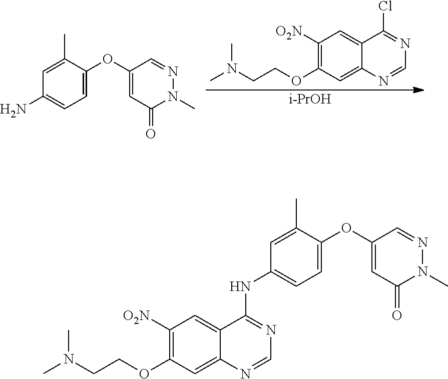

To a solution of 5-(4-amino-2-methylphenoxy)-2-methylpyridazin-3-one (325.8 mg, 1.41 mmol) in isopropyl alcohol (10.0 mL) was added {2-[(4-chloro-6-nitroquinazolin-7-yl)oxy]ethyl}dimethylamine (418.0 mg, 1.41 mmol) at 0° C. The resulting mixture was stirred at room temperature for 3 h. After the reaction was completed. the resulting mixture was concentrated under reduced pressure. The residue was purified by reverse phase flash chromatography with CH3CN/H2O (70/30, v/v) to afford 5-[4-({7-[2-(dimethylamino)ethoxy]-6-nitroquinazolin-4-yl}amino)-2-methylphenoxy]-2-methylpyridazin-3-one (114.0 mg, 16%) as a white solid. LCMS (ESI, m/z): [M+H]+=492.2.

### Step 7: Synthesis of 5-[4-({6-amino-7-[2-(dimethylamino)ethoxy]quinazolin-4-yl}amino)-2-methylphenoxy]-2-methylpyridazin-3-one

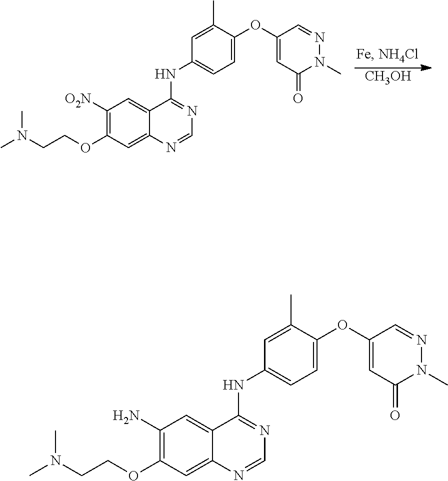

To a solution of 5-[4-({7-[2-(dimethylamino)ethoxy]-6-nitroquinazolin-4-yl}amino)-2-methylphenoxy]-2-methylpyridazin-3-one (186.8 mg, 0.38 mmol) in CH3OH/H2O (2.0/2.0 mL) was added Fe (106.1 mg, 1.90 mmol) and NH4Cl (101.7 mg, 1.90 mmol) at room temperature. The resulting mixture was stirred at 80° C. for 4 h. After the reaction was completed, the resulting mixture was filtered. The filtrate was concentrated under reduced pressure. The residue was purified by reverse phase flash chromatography with CH3CN/H2O (1/4, v/v) to afford 5-[4-({6-amino-7-[2-(dimethylamino)ethoxy]quinazolin-4-yl}amino)-2-methylphenoxy]-2-methylpyridazin-3-one (150.0 mg, 85%) as a white solid. LCMS (ESI, m/z): [M+H]+=462.2.

### Step 8: Synthesis of N-{7-[2-(dimethylamino)ethoxy]-4-({3-methyl-4-[(1-methyl-6-oxopyridazin-4-yl)oxy]phenyl}amino)quinazolin-6-yl}but-2-ynamide (Compound 201)

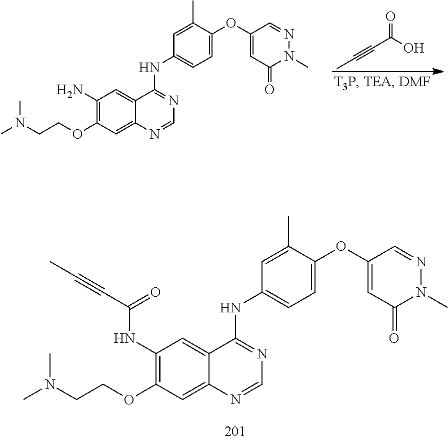

To a solution of 2-butynoic acid (32.8 mg, 0.39 mmol) in DMF (1.0 mL) was added TEA (59.2 mg, 0.59 mmol), 5-[4-({6-amino-7-[2-(dimethylamino)ethoxy]quinazolin-4-yl}amino)-2-methylphenoxy]-2-methylpyridazin-3-one (90.0 mg, 0.20 mmol) and T3P (248.2 mg, 50% in EtOAc) at room temperature. The resulting mixture was stirred at 25° C. for 1 h. After the reaction was completed, the resulting mixture was purified by reverse phase flash chromatography with CH3CN/H2O (1/5, v/v) and then purified by Prep-HPLC with the following conditions Column (XBridge Shield RP18 OBD Column, 30×150 mm, 5 μm; Mobile Phase A: Water (10 mmol/L NH4HCO3), Mobile Phase B: ACN; Flow rate: 60 mL/min; Gradient: 25% B to 45% B in 8 min; 254 nm) to afford N-{7-[2-(dimethylamino)ethoxy]-4-({3-methyl-4-[(1-methyl-6-oxopyridazin-4-yl)oxy]phenyl}amino)quinazolin-6-yl}but-2-ynamide (Compound 201) (3.4 mg, 3%) as a white solid. LCMS (ESI, m/z): [M+H]+=528.3. 1H NMR (400 MHz, DMSO-d6): δ 10.17 (s, 1H), 9.74 (s, 1H), 8.71 (s, 1H), 8.53 (s, 1H), 8.02 (d, J=2.8 Hz, 1H), 7.82-7.79 (m, 2H), 7.34 (s, 1H), 7.19 (d, J=8.8 Hz, 1H), 5.68 (d, J=2.8 Hz, 1H), 4.31-4.28 (m, 2H), 3.62 (s, 3H), 2.74-2.67 (m, 2H), 2.34 (s, 6H), 2.17 (s, 3H), 2.07 (s, 3H).

### Example S91. N-{7-[2-(dimethylamino)ethoxy]-4-[(3-fluoro-4-{[1,2,4]triazolo[1,5-a]pyridin-7-ylmethyl}phenyl)amino]quinazolin-6-yl}but-2-ynamide (Compound 202)

### Step 1: Synthesis of 7-[2-(dimethylamino)ethoxy]-N-(3-fluoro-4-{[1,5-a]pyridin-7-ylmethyl}phenyl)-6-nitroquinazolin-4-amine

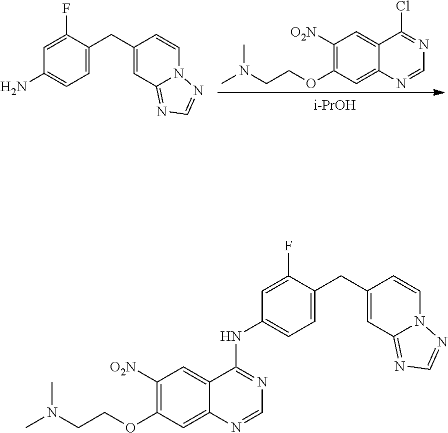

To a solution of 3-fluoro-4-{[1,2,4]triazolo[1,5-a]pyridin-7-ylmethyl}aniline (1.0 g, 4.13 mmol) in i-PrOH (40.0 mL) was added {2-[(4-chloro-6-nitroquinazolin-7-yl)oxy]ethyl}dimethylamine (1.2 g, crude) at 0° C. The resulting mixture was stirred at room temperature for 2 h. After the reaction was completed, the resulting mixture was concentrated under reduced pressure. The residue was purified by flash column chromatography with CH2Cl2/MeOH (5/1, v/v) to afford 7-[2-(dimethylamino)ethoxy]-N-(3-fluoro-4-{[1,2,4]triazolo[1,5-a]pyridin-7-ylmethyl}phenyl)-6-nitroquinazolin-4-amine (1.0 g, 48%) as a yellow solid. LCMS (ESI, m/z): [M+H]+=503.2.

### Step 2: Synthesis of 7-[2-(dimethylamino)ethoxy]-N4-(3-fluoro-4-{[1,2,4]triazolo[1,5-a]pyridin-7-ylmethyl}phenyl)quinazoline-4,6-diamine

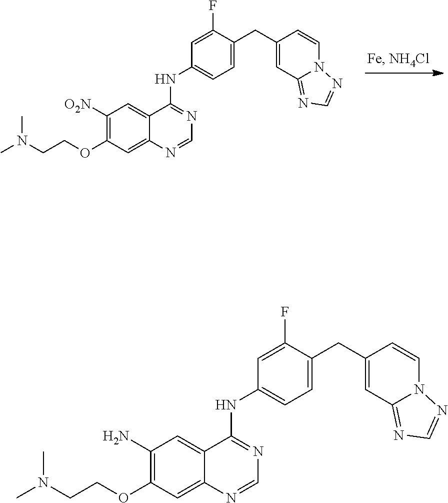 [SMILES: CN(C)CCOc1cc2ncnc(Nc3ccc(Cc4ccn5ncnc5c4)c(F)c3)c2cc1[N+](=O)[O-] ; CN(C)CCOc1cc2ncnc(Nc3ccc(Cc4ccn5ncnc5c4)c(F)c3)c2cc1N]

To a mixture of 7-[2-(dimethylamino)ethoxy]-N-(3-fluoro-4-{[1,2,4]triazolo[1,5-a]pyridin-7-ylmethyl}phenyl)-6-nitroquinazolin-4-amine (500.0 mg, 0.99 mmol) and in EtOH (20.0 mL)/H2O (4.0 mL) was added NH4Cl (159.7 mg, 2.99 mmol) at room temperature. Then Fe (166.7 mg, 2.99 mmol) was added to the mixture at 80° C. The resulting mixture was stirred at 80° C. for 3 h. After the reaction was completed, the resulting mixture was filtered. The filtrate was concentrated under reduced pressure. The residue was purified by flash column chromatography with CH2Cl2/MeOH (5/1, v/v) to afford 7-[2-(dimethylamino)ethoxy]-N4-(3-fluoro-4-{[1,2,4]triazolo[1,5-a]pyridin-7-ylmethyl}phenyl)quinazoline-4,6-diamine (300.0 mg, 63%) as a white solid. LCMS (ESI, m/z): [M+H]+=473.2.

### Step 3: Synthesis of N-{7-[2-(dimethylamino)ethoxy]-4-[(3-fluoro-4-{[1,2,4]triazolo[1,5-a]pyridin-7-ylmethyl}phenyl)amino]quinazolin-6-yl}but-2-ynamide (Compound 202)

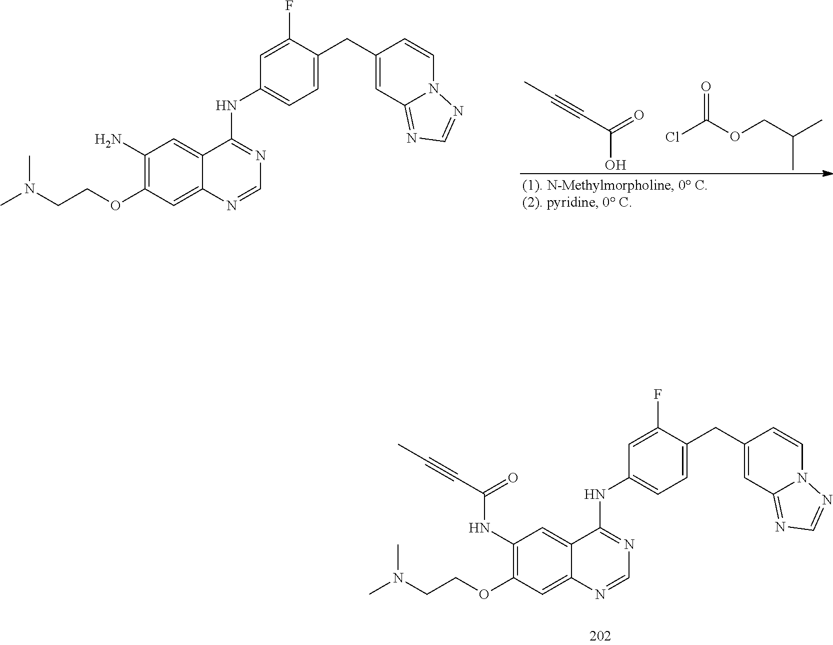

To a solution of 2-butynoic acid (42.7 mg, 0.51 mmol) in THF was added 2-methylpropyl carbonochloridate (69.4 mg, 0.51 mmol) at 0° C. The resulting mixture was stirred at 0° C. for 5 min. Then N-Methylmorpholine (128.4 mg, 1.27 mmol) was added to the mixture at 0° C. The mixture was stirred at 0° C. for 10 min. Then a solution of 7-[2-(dimethylamino)ethoxy]-N4-(3-fluoro-4-{[1,2,4]triazolo[1,5-a]pyridin-7-ylmethyl}phenyl)quinazoline-4,6-diamine (200.0 mg, 0.42 mmol) in pyridine (2.0 mL) was added dropwise to the mixture at 0° C. The mixture was stirred at 0° C. for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 5% B to 18% B in 12 min; Wave Length: 254 nm) to afford N-{7-[2-(dimethylamino)ethoxy]-4-[(3-fluoro-4-{[1,2,4]triazolo[1,5-a]pyridin-7-ylmethyl}phenyl)amino]quinazolin-6-yl}but-2-ynamide (Compound 202) (44.9 mg, 19%) as a white solid. LCMS (ESI, m/z): [M+H]+=539.3. 1H NMR (400 MHz, DMSO-d6): δ 10.39 (s, 1H), 9.80 (s, 1H), 8.87 (d, J=7.2 Hz, 1H), 8.74 (s, 1H), 8.56 (s, 1H), 8.45 (s, 1H), 8.22 (s, 1H), 7.91-7.88 (m, 1H), 7.64-7.60 (m, 2H), 7.41-7.35 (m, 1H), 7.08-7.06 (m, 1H), 4.35-4.32 (m, 2H), 4.13 (s, 2H), 2.89-2.86 (m, 2H), 2.39 (s, 6H), 2.07 (s, 3H).

### Example S92. N-{4-[(3-methyl-4-{[1,2,4]triazolo[1,5-a]pyridin-7-yloxy}phenyl)amino]-7-[2-(4-methylpiperazin-1-yl)ethoxy]quinazolin-6-yl}but-2-ynamide (Compound 203)

### Step 1: Synthesis of 7-[2-(4-methylpiperazin-1-yl)ethoxy]-6-nitro-3H-quinazolin-4-one

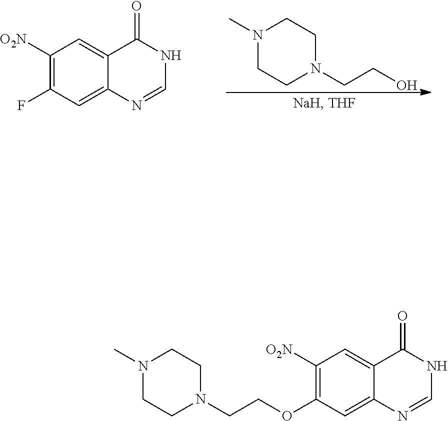

To a solution of 2-(4-methylpiperazin-1-yl)ethanol (2.1 g, 14.32 mmol) in THF (40.0 mL) was added NaH (0.5 g, 60%) at 0° C. under N2. The mixture was stirred at 0° C. for 30 min. Then 7-fluoro-6-nitro-3H-quinazolin-4-one (2.0 g, 9.56 mmol) was added to the mixture at 0° C. The mixture was stirred at room temperature for 1 h. After the reaction was completed, the resulting mixture was diluted with H₂O and extracted with DCM. The combined organic layer was washed with brine, dried over anhydrous sodium sulfate and filtered. The filtrate was concentrated under reduced pressure. The residue was purified by flash column chromatography with CH2Cl2/MeOH (5/1, v/v) to afford 7-[2-(4-methylpiperazin-1-yl)ethoxy]-6-nitro-3H-quinazolin-4-one (2.0 g, 62%) as a yellow solid. LCMS (ESI, m/z): [M+H]+=334.1.

### Step 2: Synthesis of 4-chloro-7-[2-(4-methylpiperazin-1-yl)ethoxy]-6-nitroquinazoline

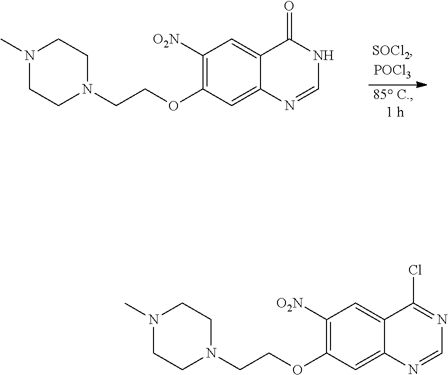 [SMILES: CN1CCN(CCOc2cc3nc[nH]c(=O)c3cc2[N+](=O)[O-])CC1 ; CN1CCN(CCOc2cc3ncnc(Cl)c3cc2[N+](=O)[O-])CC1]

To a solution of 7-[2-(4-methylpiperazin-1-yl)ethoxy]-6-nitro-3H-quinazolin-4-one (1.0 g, 3.00 mmol) in SOCl₂ (20.0 mL) was added POCl₃ (1.0 mL) and DMF (0.2 mL) at room temperature. The resulting mixture was stirred at 85° C. for 1 h. After the reaction was completed, the mixture was concentrated under vacuum to afford 4-chloro-7-[2-(4-methylpiperazin-1-yl)ethoxy]-6-nitroquinazoline (1.0 g, crude) as a brown yellow solid. LCMS (ESI, m/z): [M+H]+=352.1.

### Step 3: N-(3-methyl-4-{[1,2,4]triazolo[1,5-a]pyridin-7-yloxy}phenyl)-7-[2-(4-methylpiperazin-1-yl)ethoxy]-6-nitroquinazolin-4-amine

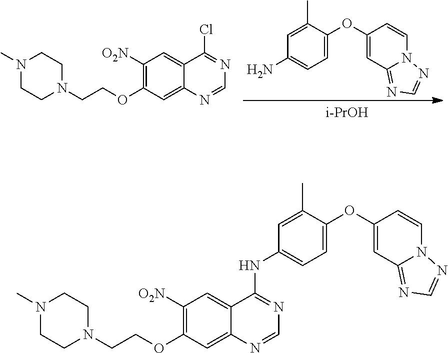 [SMILES: "i-PrOH" ; CN1CCN(CCOc2cc3ncnc(Cl)c3cc2[N+](=O)[O-])CC1 ; Cc1cc(N)ccc1Oc1ccn2ncnc2c1 ; Cc1cc(Nc2ncnc3cc(OCCN4CCN(C)CC4)c([N+](=O)[O-])cc23)ccc1Oc1ccn2ncnc2c1]

To a solution of 3-methyl-4-{[1,2,4]triazolo[1,5-a]pyridin-7-yloxy}aniline (0.7 g, 2.8 mmol) in i-PrOH (40.0 mL) was added 4-chloro-7-[2-(4-methylpiperazin-1-yl)ethoxy]-6-nitroquinazoline (1.0 g, crude) at room temperature. The resulting mixture was stirred at room temperature for 2 h. After the reaction was completed, the resulting mixture was concentrated under reduced pressure. The residue was purified by flash column chromatography with CH2Cl2/MeOH (5/1, v/v) to afford N-(3-methyl-4-{[1,2,4]triazolo[1,5-a]pyridin-7-yloxy}phenyl)-7-[2-(4-methylpiperazin-1-yl)ethoxy]-6-nitroquinazolin-4-amine (1.1 g, 69%) as a brown yellow solid. LCMS (ESI, m/z): [M+H]+=556.2.

### Step 4: N4-(3-methyl-4-{[1,2,4]triazolo[1,5-a]pyridin-7-yloxy}phenyl)-7-[2-(4-methylpiperazin-1-yl)ethoxy]quinazoline-4,6-diamine

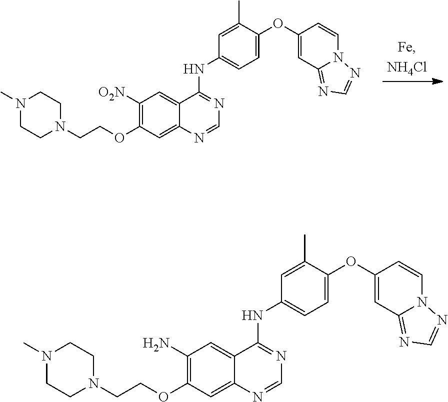 [SMILES: Cc1cc(Nc2ncnc3cc(OCCN4CCN(C)CC4)c([N+](=O)[O-])cc23)ccc1Oc1ccn2ncnc2c1 ; Cc1cc(Nc2ncnc3cc(OCCN4CCN(C)CC4)c(N)cc23)ccc1Oc1ccn2ncnc2c1]

To a mixture of N-(3-methyl-4-{[1,2,4]triazolo[1,5-a]pyridin-7-yloxy}phenyl)-7-[2-(4-methylpiperazin-1-yl)ethoxy]-6-nitroquinazolin-4-amine (1.0 g, 1.80 mmol) and NH4Cl (0.3 g, 5.40 mmol) in EtOH (20.0 mL)/H2O (4.0 mL) was added Fe (0.3 g, 5.57 mmol) at 80° C. The resulting mixture was stirred at 80° C. for 3 h. After the reaction was completed, the resulting mixture was filtered. The filtrate was concentrated under reduced pressure. The residue was purified by flash column chromatography with CH2Cl2/MeOH (5/1, v/v) to afford N4-(3-methyl-4-{[1,2,4]triazolo[1,5-a]pyridin-7-yloxy}phenyl)-7-[2-(4-methylpiperazin-1-yl)ethoxy]quinazoline-4,6-diamine (0.9 g, 95%) as a Brown yellow solid. LCMS (ESI, m/z): [M+H]+=526.3.

### Step 5: N-{4-[(3-methyl-4-{[1,2,4]triazolo[1,5-a]pyridin-7-yloxy}phenyl)amino]-7-[2-(4-methylpiperazin-1-yl)ethoxy]quinazolin-6-yl}but-2-ynamide (Compound 203)

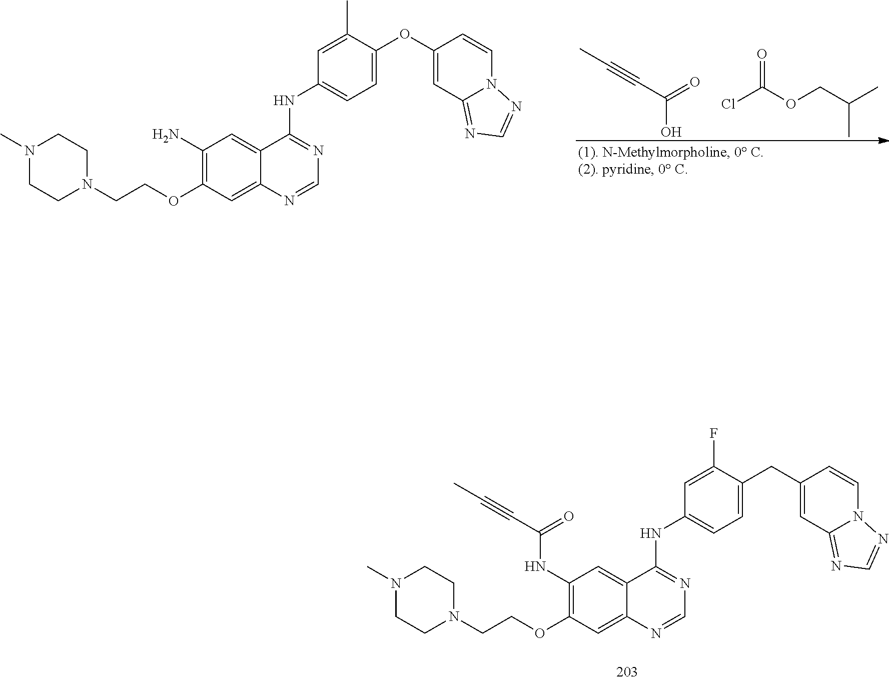

To a solution of 2-butynoic acid (41.8 mg, 0.50 mmol) in THF (10.0 mL) was added 2-methylpropyl carbonochloridate (67.9 mg, 0.50 mmol) at 0° C. The resulting mixture was stirred at 0° C. for 5 min. To the above mixture was added N-Methylmorpholine (125.7 mg, 1.24 mmol) at 0° C. The mixture was stirred at 0° C. for 30 min. Then a solution of N4-(3-methyl-4-{[1,2,4]triazolo[1,5-a]pyridin-7-yloxy}phenyl)-7-[2-(4-methylpiperazin-1-yl)ethoxy]quinazoline-4,6-diamine (200.0 mg, 0.41 mmol) in pyridine (2.0 mL) was added dropwise to the mixture at 0° C. The mixture was stirred at 0° C. for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Atlantis HILIC OBD Column, 19×150 mm, 5 um; Mobile Phase A: Water (10 mmol/L NH4HCO3), Mobile Phase B: ACN; Flow rate: 60 mL/min; Gradient: 25% B to 45% B in 11 min; Wave Length: 254 nm) to afford N-{4-[(3-methyl-4-{[1,2,4]triazolo[1,5-a]pyridin-7-yloxy}phenyl)amino]-7-[2-(4-methylpiperazin-1-yl)ethoxy]quinazolin-6-yl}but-2-ynamide (Compound 203) (42.5 mg, 18%) as a white solid. LCMS (ESI, m/z): [M+H]+=592.4. 1H NMR (400 MHz, CD3OD): δ 8.90 (s, 1H), 8.75 (d, J=7.6 Hz, 1H), 8.47 (s, 1H), 8.30 (s, 1H), 7.75 (d, J=2.0 Hz, 1H), 7.71-7.68 (m, 1H), 7.27 (s, 1H), 7.19 (d, J=8.4 Hz, 1H), 7.10-7.08 (m, 1H), 6.87 (d, J=2.4 Hz, 1H), 4.42-4.39 (m, 2H), 3.01-2.98 (m, 2H), 2.89-2.49 (m, 8H), 2.35 (s, 3H), 2.26 (s, 3H), 2.12 (s, 3H).

### Example S93. N-{7-[2-(dimethylamino)ethoxy]-4-({3-methyl-4-[(1-methyl-1,3-benzodiazol-5-yl)oxy]phenyl}amino)quinazolin-6-yl}but-2-ynamide (Compound 204)

### Step 1: Synthesis of {2-[(4-chloro-6-nitroquinazolin-7-yl)oxy]ethyl}dimethylamine

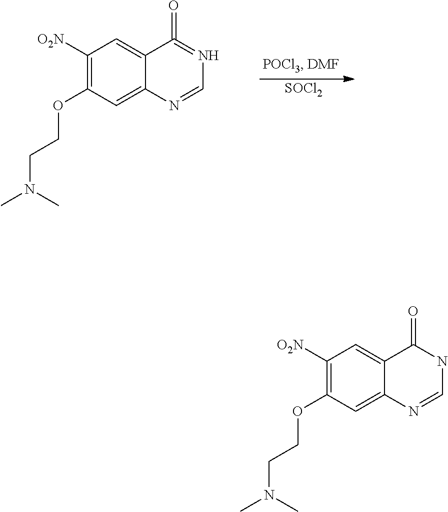

To a solution of 7-[2-(dimethylamino)ethoxy]-6-nitro-3H-quinazolin-4-one (500.0 mg, 1.79 mmol) in thionyl chloride (20.0 mL) was added POCl₃ (826.5 mg, 5.39 mmol) and DMF (0.2 mL) at room temperature. The resulting mixture was stirred at 85° C. for 1 h. After the reaction was completed, the mixture was concentrated under vacuum to afford {2-[(4-chloro-6-nitroquinazolin-7-yl)oxy]ethyl}dimethylamine (630.0 mg, crude) as a yellow solid. LCMS (ESI, m/z): [M+H]+=297.1.

### Step 2: Synthesis of 7-[2-(dimethylamino)ethoxy]-N-{3-methyl-4-[(1-methyl-1,3-benzodiazol-5-yl)oxy]phenyl}-6-nitroquinazolin-4-amine

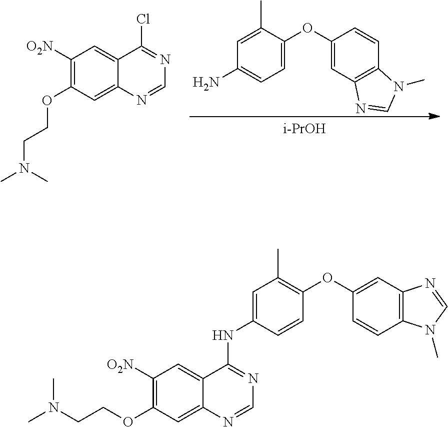 [SMILES: "i-PrOH" ; CN(C)CCOc1cc2ncnc(Cl)c2cc1[N+](=O)[O-] ; Cc1cc(N)ccc1Oc1ccc2c(c1)ncn2C ; Cc1cc(Nc2ncnc3cc(OCCN(C)C)c([N+](=O)[O-])cc23)ccc1Oc1ccc2c(c1)ncn2C]

To a solution of 3-methyl-4-[(1-methyl-1,3-benzodiazol-5-yl)oxy]aniline (500.0 mg, 1.97 mmol) in i-PrOH (20.0 mL) was added {2-[(4-chloro-6-nitroquinazolin-7-yl)oxy]ethyl}dimethylamine (585.6 mg, crude) at room temperature. The resulting mixture was stirred at room temperature for 16 h. After the reaction was completed, the resulting mixture was concentrated under vacuum. The residue was purified by flash column chromatography with ethyl acetate/petroleum ether (90/10, v/v) to afford 7-[2-(dimethylamino)ethoxy]-N-{3-methyl-4-[(1-methyl-1,3-benzodiazol-5-yl)oxy]phenyl}-6-nitroquinazolin-4-amine (550.0 mg, 48%) as a yellow solid. LCMS (ESI, m/z): [M+H]+=514.2.

### Step 3: Synthesis of 7-[2-(dimethylamino)ethoxy]-N4-{3-methyl-4-[(1-methyl-1,3-benzodiazol-5-yl)oxy]phenyl}quinazoline-4,6-diamine

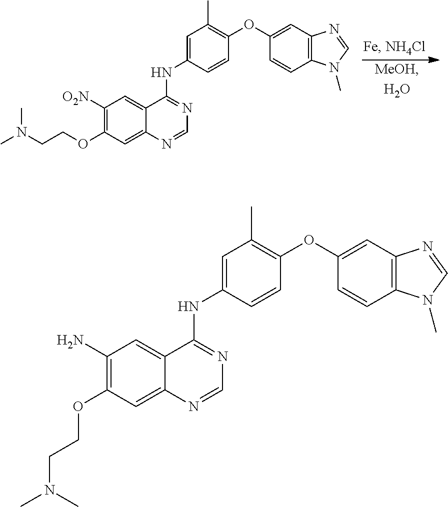 [SMILES: Cc1cc(Nc2ncnc3cc(OCCN(C)C)c([N+](=O)[O-])cc23)ccc1Oc1ccc2c(c1)ncn2C ; Cc1cc(Nc2ncnc3cc(OCCN(C)C)c(N)cc23)ccc1Oc1ccc2c(c1)ncn2C]

To a solution of 7-[2-(dimethylamino)ethoxy]-N-{3-methyl-4-[(1-methyl-1,3-benzodiazol-5-yl)oxy]phenyl}-6-nitroquinazolin-4-amine (500.0 mg, 0.97 mmol) in methanol (10.0 mL) and H2O (3.0 mL) was added Fe (543.7 mg, 9.74 mmol) and NH4Cl (520.7 mg, 9.74 mmol) at room temperature. The resulting mixture was stirred at 80° C. for 1 h. After the reaction was completed, the resulting mixture was filtered. The filtrate was concentrated under vacuum. The residue was purified by flash column chromatography with CH2Cl2/MeOH (10/1, v/v) to afford 7-[2-(dimethylamino)ethoxy]-N4-{3-methyl-4-[(1-methyl-1,3-benzodiazol-5-yl)oxy]phenyl}quinazoline-4,6-diamine (500.0 mg, 95%) as a yellow solid. LCMS (ESI, m/z): [M+H]+=484.2.

### Step 4: Synthesis of N-{7-[2-(dimethylamino)ethoxy]-4-[(3-methyl-4-[(1-methyl-1,3-benzodiazol-5-yl)oxy]phenyl}amino)quinazolin-6-yl]but-2-ynamide (Compound 204)

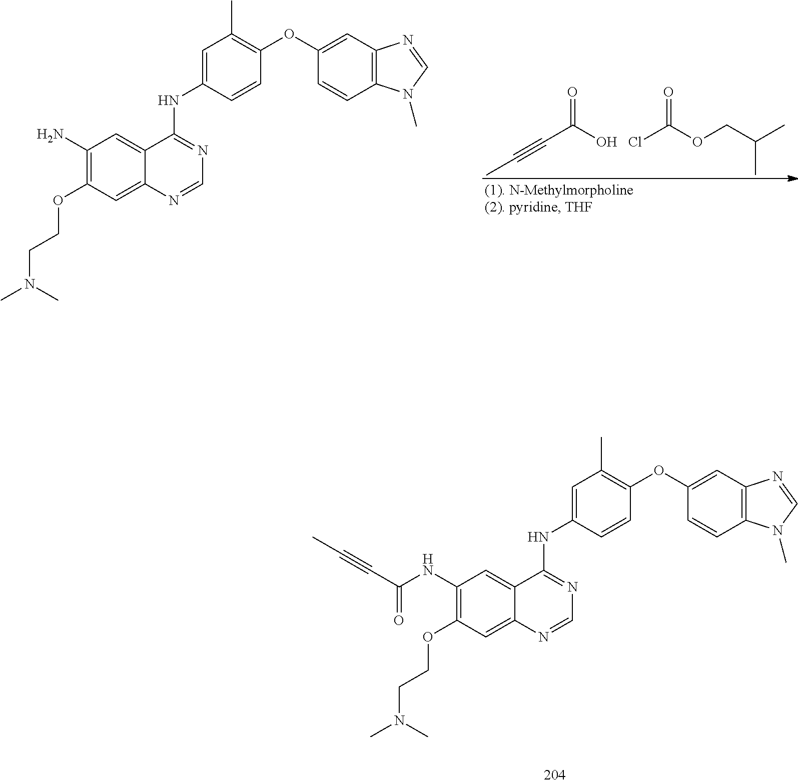

To a solution of 2-butynoic acid (434.6 mg, 5.17 mmol) in THF (15.0 mL) was added 2-methylpropyl carbonochloridate (706.1 mg, 5.17 mmol) at 0° C. The resulting mixture was stirred at 0° C. for 10 min. Then N-Methylmorpholine (1045.8 mg, 10.34 mmol) was added to the mixture at 0° C. The resulting mixture was stirred at 0° C. for another 10 min. Then a solution of 7-[2-(dimethylamino)ethoxy]-N4-{3-methyl-4-[(1-methyl-1,3-benzodiazol-5-yl)oxy]phenyl}quinazoline-4,6-diamine (500.0 mg, 1.034 mmol) in pyridine (10.0 mL) was added dropwise to the mixture at 0° C. The resulting mixture was stirred at 0° C. for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) and then purified by Prep-HPLC with the following conditions Column (Xselect CSH OBD Column 30×150 mm, 5 um; Mobile Phase A: Water (0.1% FA), Mobile Phase B: ACN; Flow rate: 60 mL/min; Gradient: 3% B to 12% B in 12 min; Wave Length: 254 nm) to afford N-{7-[2-(dimethylamino)ethoxy]-4-({3-methyl-4-[(1-methyl-1,3-benzodiazol-5-yl)oxy]phenyl}amino)quinazolin-6-yl}but-2-ynamide (Compound 204) (5.3 mg, 1%) as a white solid. LCMS (ESI, m/z): [M+H]⁺=550.4.41 NMR (400 MHz, DMSO-d₆): δ 10.21 (s, 1H), 9.65 (s, 1H), 8.69 (s, 1H), 8.48 (s, 1H), 8.17 (s, 1H), 7.71 (s, 1H), 7.61-7.56 (m, 2H), 7.32 (s, 1H), 7.10 (d, J=2.0 Hz, 1H), 7.01-6.99 (m, 1H), 6.86 (d, J=8.0 Hz, 1H), 4.30-4.27 (m, 2H), 3.84 (s, 3H), 2.73-2.68 (m, 2H), 2.29 (s, 6H), 2.24 (s, 3H), 2.08 (s, 3H).

### Example S94. N-{7-[2-(dimethylamino)ethoxy]-5-fluoro-4-{[3-methyl-4-(pyridin-3-yloxy)phenyl}amino]quinazolin-6-yl}but-2-ynamide (Compound 205)

### Step 1: Synthesis of 2-azido-6-fluoro-4-hydroxybenzonitrile

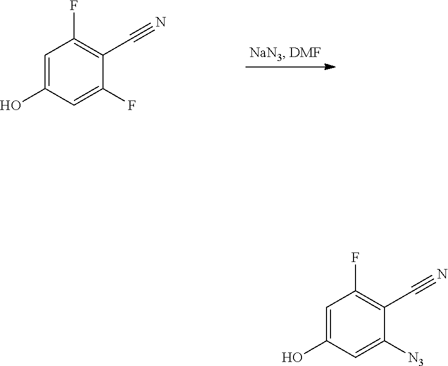 [SMILES: N#Cc1c(F)cc(O)cc1F ; N#Cc1c(F)cc(O)cc1N=[N+]=[N-]]

To a solution of 2,6-difluoro-4-hydroxybenzonitrile (25.0 g, 161.18 mmol) in DMF (250.0 mL) was added NaN₃ (11.5 g, 177.35 mmol) at room temperature. The resulting mixture was stirred at 70° C. for 16 h. After the reaction was completed, the pH value of the mixture was adjusted to 7 with HCl (1 mol/L) and then extracted with ethyl acetate. The combined organic layers were washed with brine, dried over anhydrous Na₂SO₄ and filtered. The filtrate was concentrated under reduced pressure. The residue was purified by flash column chromatography with petroleum ether/ethyl acetate (1/1, v/v) to afford 2-azido-6-fluoro-4-hydroxybenzonitrile (10.0 g, 24%) as an off-white solid. LCMS (ESI, m/z): [M+H]⁺=179.0.

### Step 2: Synthesis of 2-amino-6-fluoro-4-hydroxybenzonitrile

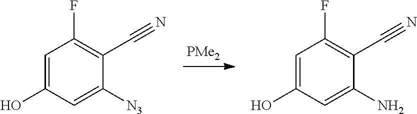 [SMILES: N#Cc1c(F)cc(O)cc1N=[N+]=[N-] ; N#Cc1c(N)cc(O)cc1F]

To a solution of 2-azido-6-fluoro-4-hydroxybenzonitrile (5.0 g, 28.09 mmol) in THF (50.0 mL) was added dropwise trimethylphosphine (30.0 mL, 2 mol/L) at 0° C. The resulting mixture was stirred at room temperature for 2 h. After the reaction was completed, the resulting mixture was quenched with water and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 2-amino-6-fluoro-4-hydroxybenzonitrile (1.2 g, 28%) as a light yellow solid. LCMS (ESI, m/z): [M+H]+=153.0.

### Step 3: Synthesis of 6-amino-2-fluoro-4-hydroxy-3-iodobenzonitril

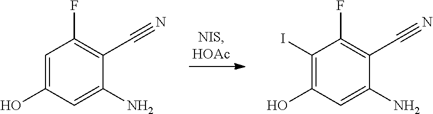 [SMILES: N#Cc1c(N)cc(O)cc1F ; N#Cc1c(N)cc(O)c(I)c1F]

A solution of 2-amino-6-fluoro-4-hydroxybenzonitrile (3.0 g, 19.71 mmol) and MS (4.4 g, 19.71 mmol) in HOAc (10.0 mL) was stirred at 50° C. for 16 h. After the reaction was completed, the mixture was concentrated under reduced pressure. The residue was purified by flash column chromatography with petroleum ether/ethyl acetate (7/3, v/v) to afford 6-amino-2-fluoro-4-hydroxy-3-iodobenzonitrile (1.0 g, 18%) as a yellow solid. LCMS (ESI, m/z): [M+H]⁺=278.9.

### Step 4: Synthesis of (E)-N′-(2-cyano-3-fluoro-5-hydroxy-4-iodophenyl)-N,N-dimethylmethanimidamide

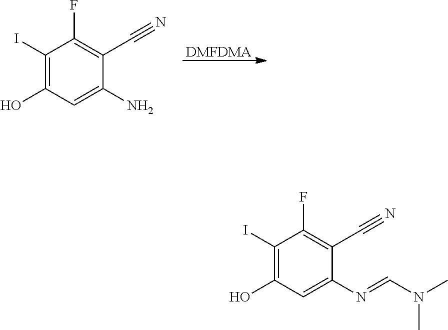

To a mixture of 6-amino-2-fluoro-4-hydroxy-3-iodobenzonitrile (700.0 mg, 2.52 mmol) in MeOH (20.0 mL) was added DMF-DMA (450.0 mg, 3.77 mmol) at room temperature. The resulting mixture was stirred at 70° C. for 1 h. After the reaction was completed, the mixture was concentrated under reduced pressure to afford (E)-N′-(2-cyano-3-fluoro-5-hydroxy-4-iodophenyl)-N,N-dimethylmethanimidamide (850.0 mg, crude) as a brown solid. LCMS (ESI, m/z): [M+H]+=334.0.

### Step 5: Synthesis of 3-(2-methyl-4-nitrophenoxy)pyridine

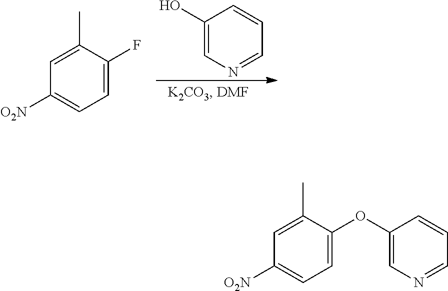 [SMILES: Cc1cc([N+](=O)[O-])ccc1F ; Oc1cccnc1 ; Cc1cc([N+](=O)[O-])ccc1Oc1cccnc1]

To a solution of 1-fluoro-2-methyl-4-nitrobenzene (5.0 g, 32.23 mmol) in DMF (150.0 mL) was added K₂CO₃ (8.9 g, 64.39 mmol) and 3-hydroxypyridine (3.0 g, 32.17 mmol) at room temperature. The resulting mixture was stirred at 50° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (5/1, v/v) to afford 3-(2-methyl-4-nitrophenoxy)pyridine (5.6 g, 76%) as a yellow solid. LCMS (ESI, m/z): [M+H]+=231.1.

### Step 6: Synthesis of 3-methyl-4-(pyridin-3-yloxy)aniline

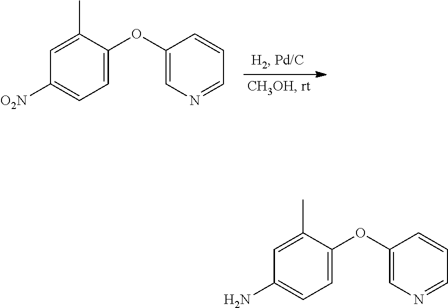 [SMILES: Cc1cc([N+](=O)[O-])ccc1Oc1cccnc1 ; Cc1cc(N)ccc1Oc1cccnc1]

To a solution of 3-(2-methyl-4-nitrophenoxy)pyridine (5.6 g, 24.32 mmol) in MeOH (100.0 mL) was added Pd/C (1.7 g, dry) at room temperature. The resulting mixture was stirred at room temperature for 3 h under H2. After the reaction was completed, the resulting mixture was filtered. The filtrate was concentrated under reduced pressure. The residue was purified by flash column chromatography with CH₂Cl₂/MeOH (10/1, v/v) to afford 3-methyl-4-(pyridin-3-yloxy)aniline (3.7 g, 75%) as a yellow solid. LCMS (ESI, m/z): [M+H]+=201.1.

### Step 7: Synthesis of 5-fluoro-6-iodo-4-{[3-methyl-4-(pyridin-3-yloxy)phenyl]amino}quinazolin-7-ol

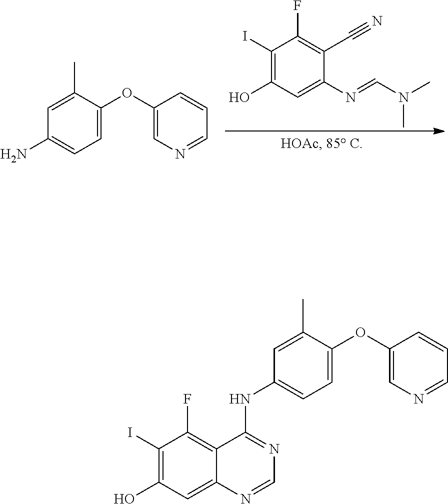

To a solution of 3-methyl-4-(pyridin-3-yloxy)aniline (750.0 mg, 3.74 mmol) in HOAc (10.0 mL) was added (E)-N′-(2-cyano-3-fluoro-5-hydroxy-4-iodophenyl)-N,N-dimethylmethanimidamide (1247.6 mg, 3.75 mmol) at room temperature. The resulting mixture was stirred at 85° C. for 1 h. The mixture was concentrated under reduced pressure. The residue was purified by flash column chromatography with CH₂Cl₂/MeOH (5/1, v/v) to afford 5-fluoro-6-iodo-4-{[3-methyl-4-(pyridin-3-yloxy)phenyl]amino}quinazolin-7-ol (800.0 mg, 43%) as a brown solid. LCMS (ESI, m/z): [M+H]+=489.0.

### Step 8: Synthesis of 7-[2-(dimethylamino)ethoxy]-5-fluoro-6-iodo-N-[3-methyl-4-(pyridin-3-yloxy)phenyl]quinazolin-4-amine

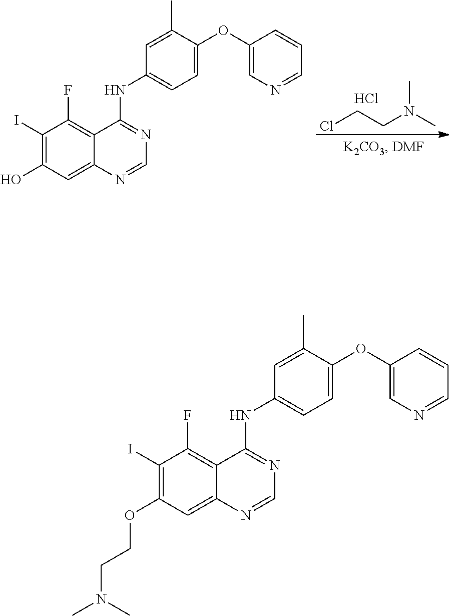

To a solution of 5-fluoro-6-iodo-4-{[3-methyl-4-(pyridin-3-yloxy)phenyl]amino}quinazolin-7-ol (550.0 mg, 1.13 mmol) in DMF (10.0 mL) was added dimethylaminoethyl chloride hydrochloride (162.2 mg, 1.13 mmol), K2CO3 (622.7 mg, 4.50 mmol) and KI (22.4 mg, 0.14 mmol) at room temperature. The resulting mixture was stirred at 50° C. for 16 h. After the reaction was completed, the resulting mixture was purified by reverse phase flash chromatography with MeOH/H₂O (60/40, v/v) to afford 7-[2-(dimethylamino)ethoxy]-5-fluoro-6-iodo-N-[3-methyl-4-(pyridin-3-yloxy)phenyl]quinazolin-4-amine (200.0 mg, 31%) as an off-white solid. LCMS (ESI, m/z): [M+H]+=560.1

### Step 9: Synthesis of 7-[2-(dimethylamino)ethoxy]-N6-(diphenylmethylidene)-5-fluoro-N4-[3-methyl-4-(pyridin-3-yloxy)phenyl]quinazoline-4,6-diamine

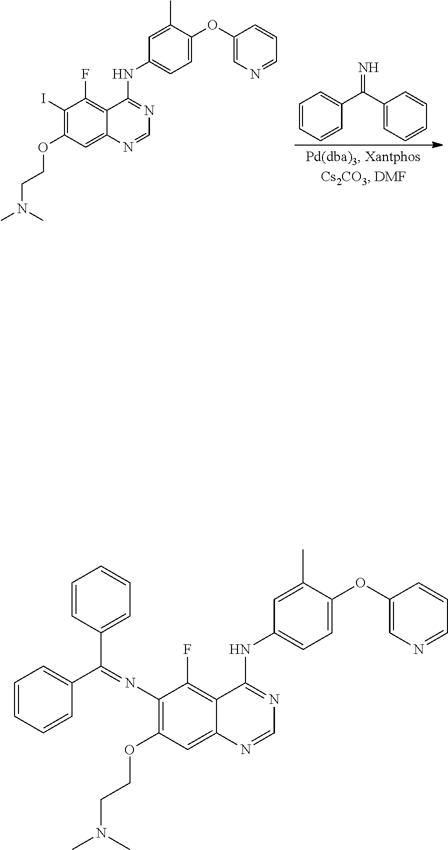 [SMILES: Cc1cc(Nc2ncnc3cc(OCCN(C)C)c(I)c(F)c23)ccc1Oc1cccnc1 ; N=C(c1ccccc1)c1ccccc1 ; Cc1cc(Nc2ncnc3cc(OCCN(C)C)c(N=C(c4ccccc4)c4ccccc4)c(F)c23)ccc1Oc1cccnc1]

To a solution of 7-[2-(dimethylamino)ethoxy]-5-fluoro-6-iodo-N-[3-methyl-4-(pyridin-3-yloxy)phenyl]quinazolin-4-amine (200.0 mg, 0.36 mmol) in DMF (8.0 mL) was added diphenylmethanimine (129.6 mg, 0.72 mmol), Cs2CO3 (233.0 mg, 0.72 mmol), Xantphos (41.3 mg, 0.072 mmol) and Pd₂(dba)₃ (5.1 mg, 0.01 mmol) at room temperature under N₂. The resulting mixture was stirred at 100° C. for 16 h. After the reaction was completed, the mixture was diluted with H2O and extracted with ethyl acetate. The combined organic layer was washed with brine, dried over anhydrous sodium sulfate and filtered. The filtrate was concentrated under reduced pressure. The residue was purified by reverse phase flash chromatography with MeOH/H₂O (60/40, v/v) to afford 7-[2-(dimethylamino)ethoxy]-N6-(diphenylmethylidene)-5-fluoro-N4-[3-methyl-4-(pyridin-3-yloxy)phenyl]quinazoline-4,6-diamine (150.0 mg, 68%) as an off-white solid. LCMS (ESI, m/z): [M+H]+=613.3.

### Step 10: Synthesis of 7-[2-(dimethylamino)ethoxy]-5-fluoro-N4-[3-methyl-4-(pyridin-3-yloxy)phenyl]quinazoline-4,6-diamine

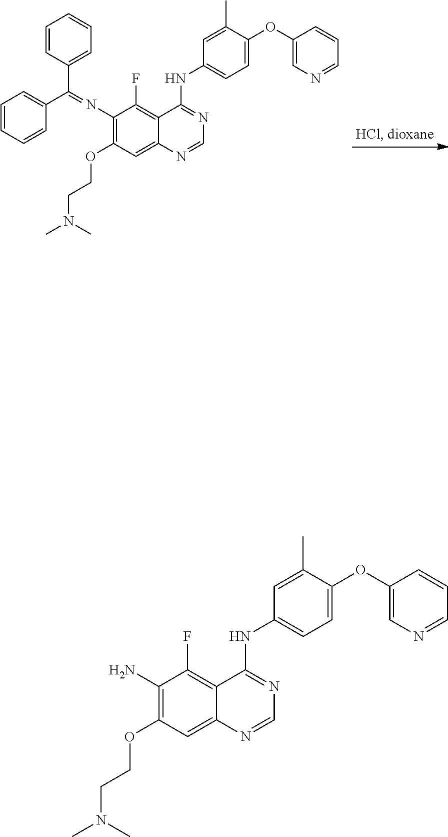 [SMILES: Cc1cc(Nc2ncnc3cc(OCCN(C)C)c(N=C(c4ccccc4)c4ccccc4)c(F)c23)ccc1Oc1cccnc1 ; Cc1cc(Nc2ncnc3cc(OCCN(C)C)c(N)c(F)c23)ccc1Oc1cccnc1]

The mixture of 7-[2-(dimethylamino)ethoxy]-N6-(diphenylmethylidene)-5-fluoro-N4-[3-methyl-4-(pyridin-3-yloxy)phenyl]quinazoline-4,6-diamine (150.0 mg, 0.25 mmol) in HCl/dioxane (5.0 mL, 4 mol/L) was stirred at room temperature for 1 h. After the reaction was completed, the pH value of the mixture was adjusted to 8 with NaHCO₃. The mixture was diluted with H₂O and extracted with CH₂Cl₂. The combined organic layer was washed with brine, dried over Na₂SO₄ and filtered. The filtrate was evaporated in vacuo. The residue was purified by reverse phase flash chromatography with MeOH/H₂O (60/40, v/v) to afford 7-[2-(dimethylamino)ethoxy]-5-fluoro-N4-[3-methyl-4-(pyridin-3-yloxy)phenyl]quinazoline-4,6-diamine (44.0 mg, 40%) as an off-white solid. LCMS (ESI, m/z): [M+H]+=449.2.

### Step 11: Synthesis of N-{7-[2-(dimethylamino)ethoxy]-5-fluoro-4-{[3-methyl-4-(pyridin-3-yloxy)phenyl]amino}quinazolin-6-yl}but-2-ynamide (Compound 205)

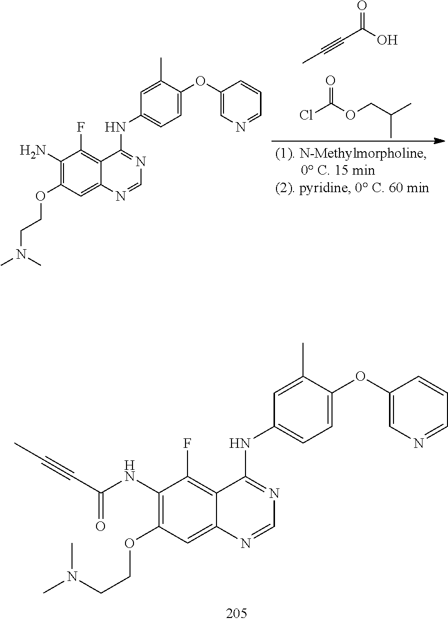

To a solution of 2-butynoic acid (471.7 mg, 5.61 mmol) in THF (10.0 mL) was added 2-methylpropyl carbonochloridate (1010.1 mg, 7.39 mmol) at 0 C under N₂. The resulting mixture was stirred at 0 C for 5 min. To the above mixture was added N-Methylmorpholine (1031.8 mg, 10.20 mmol) at 0 C under N₂. The mixture was stirred at 0 C for 10 min. Then a solution of 7-[2-(dimethylamino)ethoxy]-5-fluoro-N4-[3-methyl-4-(pyridin-3-yloxy)phenyl]quinazoline-4,6-diamine (600.0 mg, 1.27 mmol) in pyridine (10.0 mL) was added dropwise to the mixture at 0 C. The mixture was stirred at 0 C for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 25% B to 50% B in 8 min; Wave Length: 254 nm) to afford N-{7-[2-(dimethylamino)ethoxy]-5-fluoro-4-{[3-methyl-4-(pyridin-3-yloxy)phenyl]amino}quinazolin-6-yl}but-2-ynamide (Compound 205) (0.4 mg, 1%) as a white solid. LCMS (ESI, m/z): [M+H]+=515.1. ¹H NMR (400 MHz, CD₃OD): δ 8.46 (s, 1H), 8.31-8.27 (m, 2H), 7.65 (d, J=2.4 Hz, 1H), 7.60-7.57 (m, 1H), 7.45-7.39 (m, 2H), 7.13 (s, 1H), 7.06 (d, J=8.4 Hz, 1H), 4.35-4.33 (m, 2H), 2.92-2.90 (m, 2H), 2.44 (s, 6H), 2.27 (s, 3H), 2.11 (s, 3H).

### Example S95. N-(7-(2-(dimethylamino)ethoxy)-4-((4-((1-ethyl-2-oxo-1,2-dihydropyridin-4-yl)oxy)-3-methylphenyl)amino)quinazolin-6-yl)but-2-ynamide (Compound 206)

### Step 1: Synthesis of 4-(benzyloxy)-1-ethylpyridin-2(1H)-one

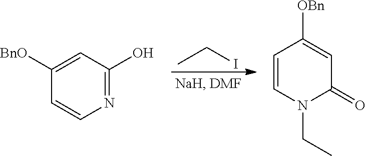

To a solution of 4-(benzyloxy)pyridin-2-ol (10.0 g, 49.70 mmol) in DMF (100.0 mL) was added NaH (3.6 g, 60%) at 0° C. under N₂. The resulting mixture was stirred at 0° C. for 1 h. Then iodoethane (11.6 g, 75.54 mmol) was added dropwise to the mixture at 0° C. The resulting mixture was stirred at room temperature for 16 h. After the reaction was completed, the resulting mixture was quenched with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (10/1, v/v) to afford 4-(benzyloxy)-1-ethylpyridin-2(1H)-one (6.8 g, 59%) as a yellow oil. LCMS (ESI, m/z): [M+H]⁺=230.1.

### Step 2: Synthesis of 1-ethyl-4-hydroxypyridin-2(1H)-one

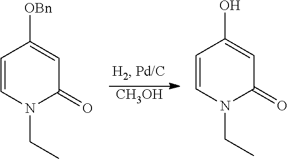

To a solution of 4-(benzyloxy)-1-ethylpyridin-2(1H)-one (4.8 g, 20.93 mmol) in CH₃OH (100.0 mL) was added Pd/C (1.4 g, dry) at room temperature under N₂. The resulting mixture was stirred at room temperature for 3 h under H₂. After the reaction was completed, the resulting mixture was filtered. The filtrate was concentrated under reduced pressure to afford 1-ethyl-4-hydroxypyridin-2(1H)-one (2.2 g, crude) as a white solid. LCMS (ESI, m/z): [M+H]⁺=140.1.

### Step 3: Synthesis of 1-ethyl-4-(2-methyl-4-nitrophenoxy)pyridin-2(1H)-one

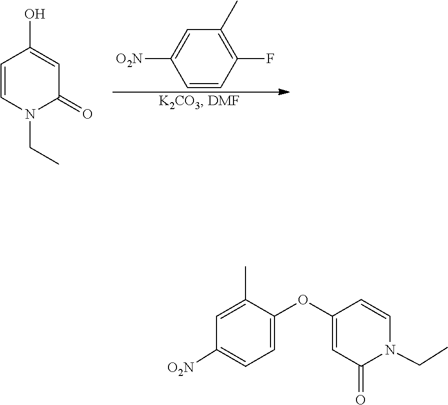

To a solution of 1-ethyl-4-hydroxypyridin-2(1H)-one (2.2 g, crude) in DMF (50.0 mL) was added 1-fluoro-2-methyl-4-nitrobenzene (2.5 g, 15.81 mmol) and K₂CO₃ (4.4 g, 31.62 mmol) room temperature. The resulting mixture was stirred at 50° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford 1-ethyl-4-(2-methyl-4-nitrophenoxy)pyridin-2(1H)-one (2.4 g, 55%) as a white solid. LCMS (ESI, m/z): [M+H]⁺=275.1.

### Step 4: Synthesis of 4-(4-amino-2-methylphenoxy)-1-ethylpyridin-2(1H)-one

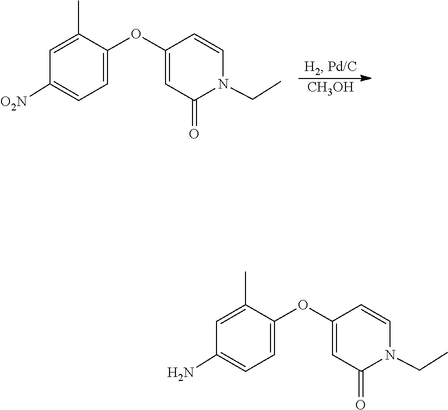

To a solution of 1-ethyl-4-(2-methyl-4-nitrophenoxy)pyridin-2(1H)-one (2.4 g, 8.75 mmol) in CH₃OH (50.0 mL) was added Pd/C (720.0 mg, dry) at room temperature. The resulting mixture was stirred at room temperature for 3 h under N₂. After the reaction was completed, the resulting mixture was filtered. The filtrate was concentrated under reduced pressure to afford 4-(4-amino-2-methylphenoxy)-1-ethylpyridin-2(1H)-one (2.0 g, crude) as a white solid. LCMS (ESI, m/z): [M+H]⁺=245.1.

### Step 5: Synthesis of 4-(4-((7-(2-(dimethylamino)ethoxy)-6-nitroquinazolin-4-yl)amino)-2-methylphenoxy)-1-ethylpyridin-2(1H)-one

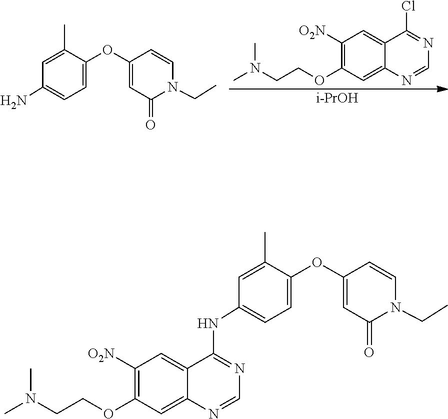

To a solution of 4-(4-amino-2-methylphenoxy)-1-ethylpyridin-2(1H)-one (580.0 mg, crude) in i-PrOH (10.0 mL) was added 2-(4-chloro-6-nitroquinazolin-7-yloxy)-N,N-dimethylethanamine (700.0 mg, 2.36 mmol) at room temperature. The resulting mixture was stirred at room temperature for 2 h. After the reaction was completed, the resulting mixture was concentrated under reduced pressure. The residue was purified by flash column chromatography with CH₂Cl₂/CH₃OH (5/1, v/v) to afford 4-(4-((7-(2-(dimethylamino)ethoxy)-6-nitroquinazolin-4-yl)amino)-2-methylphenoxy)-1-ethylpyridin-2(1H)-one (1.3 g, 86%) as a yellow solid. LCMS (ESI, m/z): [M+H]⁺=505.2.

### Step 6: Synthesis of 4-(4-((6-amino-7-(2-(dimethylamino)ethoxy)quinazolin-4-yl)amino)-2-methylphenoxy)-1-ethylpyridin-2(1H)-one

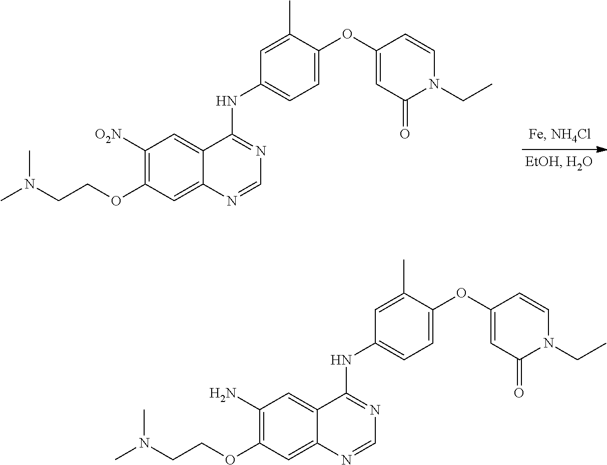

To a solution of 4-(4-((7-(2-(dimethylamino)ethoxy)-6-nitroquinazolin-4-yl)amino)-2-methylphenoxy)-1-ethylpyridin-2(1H)-one (800.0 mg, 1.57 mmol) in EtOH/H₂O (16.0/4.0 mL) was added NH₄Cl (339.3 mg, 6.34 mmol) at room temperature. The Fe (265.6 mg, 4.76 mmol) was added to the mixture at 80° C. The resulting mixture was stirred at 80° C. for 4 h. After the reaction was completed, the resulting mixture was filtered. The filtrate was concentrated under reduced pressure. The residue was purified by flash column chromatography with CH₂Cl₂/CH₃OH (7/3, v/v) to afford 4-(4-((6-amino-7-(2-(dimethylamino)ethoxy)quinazolin-4-yl)amino)-2-methylphenoxy)-1-ethylpyridin-2(1H)-one (530.0 mg, 70%) as a yellow solid. LCMS (ESI, m/z): [M+H]⁺=475.2.

### Step 7: Synthesis of N-(7-(2-(dimethylamino)ethoxy)-4-((4-((1-ethyl-2-oxo-1,2-dihydropyridin-4-yl)oxy)-3-methylphenyl)amino)quinazolin-6-yl)but-2-ynamide (Compound 206)

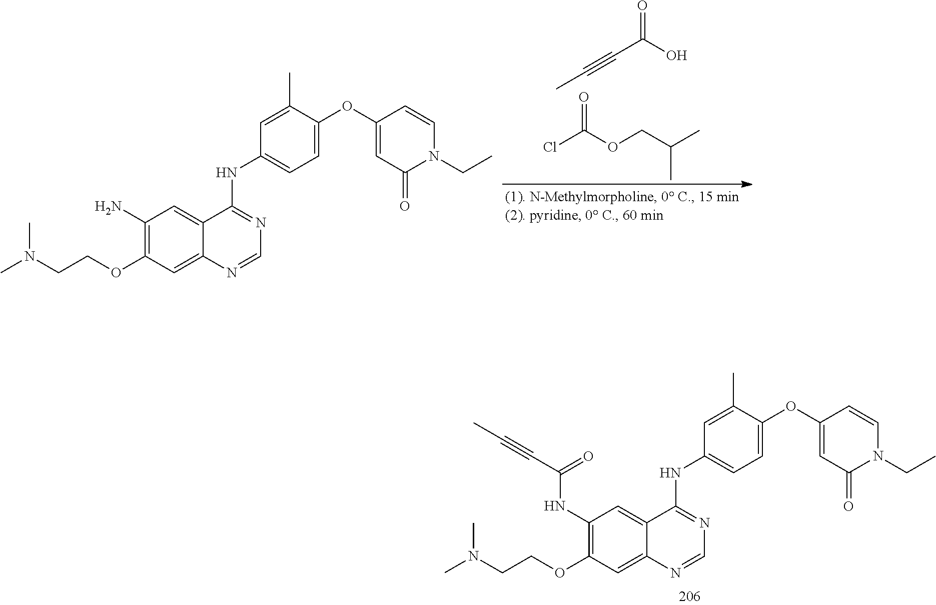

To a solution of but-2-ynoic acid (177.2 mg, 2.11 mmol) in THF (10.0 mL) was added isobutyl carbonochloridate (287.8 mg, 2.11 mmol) at 0° C. under N₂. The resulting mixture was stirred at 0° C. for 5 min. Then N-Methylmorpholine (426.3 mg, 4.21 mmol) was added to the mixture at 0° C. The resulting mixture was stirred at 0° C. for 10 min. Then a solution of 4-(4-((6-amino-7-(2-(dimethylamino)ethoxy)quinazolin-4-yl)amino)-2-methylphenoxy)-1-ethylpyridin-2(1H)-one (200.0 mg, 0.42 mmol) in pyridine (1.0 mL) was added dropwise to the mixture at 0° C. under N₂. The resulting mixture was stirred at 0° C. for another 30 min. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (5/1, v/v) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 23% to 43% in 8 min, 254 nm) to afford N-(7-(2-(dimethylamino)ethoxy)-4-((4-((1-ethyl-2-oxo-1,2-dihydropyridin-4-yl)oxy)-3-methylphenyl)amino)quinazolin-6-yl)but-2-ynamide (Compound 206) (25.8 mg, 11%) as a white solid. LCMS (ESI, m/z): [M+H]⁺=541.4. ¹H NMR (400 MHz, DMSO-d₆): δ 10.21 (s, 1H), 9.72 (s, 1H), 8.71 (s, 1H), 8.52 (s, 1H), 7.77-7.72 (m, 3H), 7.34 (s, 1H), 7.11 (d, J=8.4 Hz, 1H), 6.12-6.10 (m, 1H), 5.33 (d, J=2.8 Hz, 1H), 4.30-4.28 (m, 2H), 3.88-3.83 (m, 2H), 2.73-2.67 (m, 2H), 2.28 (s, 6H), 2.20 (s, 3H), 2.12 (s, 3H), 1.24-1.17 (m, 3H).

### Example S96. N-(4-((4-([1,2,4]triazolo[1,5-a]pyridin-7-yloxy)-3-methylphenyl)amino)-3-cyano-7-(2-(dimethylamino)ethoxy)quinolin-6-yl)but-2-ynamide (Compound 207)

### Step 1: Synthesis of N-(4-((4-([1,2,4]triazolo[1,5-a]pyridin-7-yloxy)-3-methylphenyl)amino)-3-cyano-7-ethoxyquinolin-6-yl)acetamide

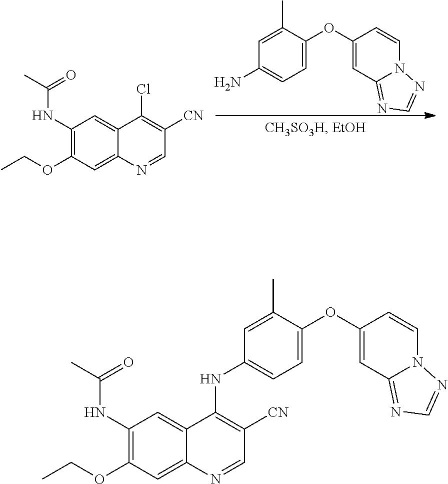 [SMILES: CCOc1cc2ncc(C#N)c(Cl)c2cc1NC(C)=O ; Cc1cc(N)ccc1Oc1ccn2ncnc2c1 ; CCOc1cc2ncc(C#N)c(Nc3ccc(Oc4ccn5ncnc5c4)c(C)c3)c2cc1NC(C)=O]

To a solution of N-(4-chloro-3-cyano-7-ethoxyquinolin-6-yl)acetamide (1.0 g, 3.45 mmol) in EtOH (20.0 mL) was added 4-([1,2,4]triazolo[1,5-a]pyridin-7-yloxy)-3-methylaniline (0.8 g, 3.45 mmol) and CH₃SO₃H (0.2 g, 1.73 mmol) at room temperature under N₂. The resulting mixture was stirred at 80° C. for 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (10/1, v/v) to afford N-(4-((4-([1,2,4]triazolo[1,5-a]pyridin-7-yloxy)-3-methylphenyl)amino)-3-cyano-7-ethoxyquinolin-6-yl)acetamide (1.3 g, 76%) as a yellow solid. LCMS (ESI, m/z): [M+H]⁺=494.2.

### Step 2: Synthesis of N-(4-((4-([1,2,4]triazolo[1,5-a]pyridin-7-yloxy)-3-methylphenyl)amino)-3-cyano-7-hydroxyquinolin-6-yl)acetamide

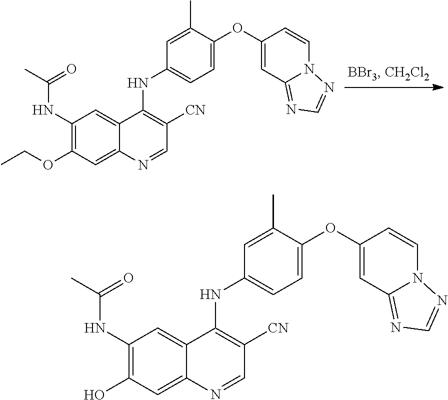 [SMILES: CCOc1cc2ncc(C#N)c(Nc3ccc(Oc4ccn5ncnc5c4)c(C)c3)c2cc1NC(C)=O ; CC(=O)Nc1cc2c(Nc3ccc(Oc4ccn5ncnc5c4)c(C)c3)c(C#N)cnc2cc1O]

To a solution of N-(4-((4-([1,2,4]triazolo[1,5-a]pyridin-7-yloxy)-3-methylphenyl)amino)-3-cyano-7-ethoxyquinolin-6-yl)acetamide (1.4 g, 2.84 mmol) in CH₂Cl₂ (20.0 mL) was added dropwise BBr₃ (8.5 mL, 1 mol/L) at room temperature. The resulting mixture was stirred at room temperature for 16 h. After the reaction was completed, the resulting mixture was concentrated under reduced pressure. The residue was purified by flash column chromatography with CH₂Cl₂/CH₃OH (10/1, v/v) to afford N-(4-((4-([1,2,4]triazolo[1,5-a]pyridin-7-yloxy)-3-methylphenyl)amino)-3-cyano-7-hydroxyquinolin-6-yl)acetamide (220.0 mg, 36%) as a yellow solid. LCMS (ESI, m/z): [M+H]⁺=466.2.

### Step 3: Synthesis of N-(4-((4-([1,2,4]triazolo[1,5-a]pyridin-7-yloxy)-3-methylphenyl)amino)-3-cyano-7-(2-(dimethylamino)ethoxy)quinolin-6-yl)acetamide

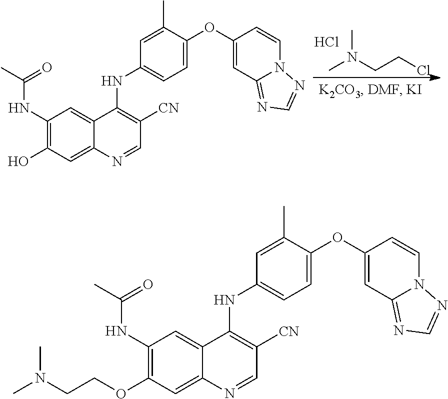

To a solution of N-(4-((4-([1,2,4]triazolo[1,5-a]pyridin-7-yloxy)-3-methylphenyl)amino)-3-cyano-7-hydroxyquinolin-6-yl)acetamide (670.0 mg, 1.44 mmol) in DMF (20.0 mL) was added 2-chloro-N,N-dimethylethan-1-amine hydrochloride (370.4 mg, 2.59 mmol), K₂CO₃ (596.8 mg, 4.32 mmol) and KI (47.8 mg, 0.29 mmol) at room temperature. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (10/1, v/v) to afford N-(4-((4-([1,2,4]triazolo[1,5-a]pyridin-7-yloxy)-3-methylphenyl)amino)-3-cyano-7-(2-(dimethylamino)ethoxy)quinolin-6-yl)acetamide (160.0 mg, 20%) as a yellow solid. LCMS (ESI, m/z): [M+H]⁺=537.2.

### Step 4: Synthesis of 4-((4-([1,2,4]triazolo[1,5-a]pyridin-7-yloxy)-3-methylphenyl)amino)-6-amino-7-(2-(dimethylamino)ethoxy)quinoline-3-carbonitrile

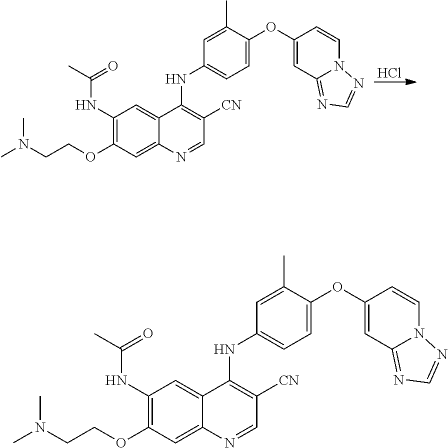 [SMILES: CC(=O)Nc1cc2c(Nc3ccc(Oc4ccn5ncnc5c4)c(C)c3)c(C#N)cnc2cc1OCCN(C)C ; Cl ; CC(=O)Nc1cc2c(Nc3ccc(Oc4ccn5ncnc5c4)c(C)c3)c(C#N)cnc2cc1OCCN(C)C]

A solution of N-(4-((4-([1,2,4]triazolo[1,5-a]pyridin-7-yloxy)-3-methylphenyl)amino)-3-cyano-7-(2-(dimethylamino)ethoxy)quinolin-6-yl)acetamide (160.0 mg, 0.30 mmol) in HCl (5.0 mL) was stirred at 100° C. for 4 h. After the reaction was completed, the resulting mixture was diluted with H₂O. The mixture was adjusted pH to 8 with aq. NaHCO₃ and then extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (10/1, v/v) to afford 4-((4-([1,2,4]triazolo[1,5-a]pyridin-7-yloxy)-3-methylphenyl)amino)-6-amino-7-(2-(dimethylamino)ethoxy)quinoline-3-carbonitrile (100.0 mg, 67%) as a yellow solid. LCMS (ESI, m/z): [M+H]⁺=495.2.

### Step 5: Synthesis of N-(4-((4-([1,2,4]triazolo[1,5-a]pyridin-7-yloxy)-3-methylphenyl)amino)-3-cyano-7-(2-(dimethylamino)ethoxy)quinolin-6-yl)but-2-ynamide (Compound 207)

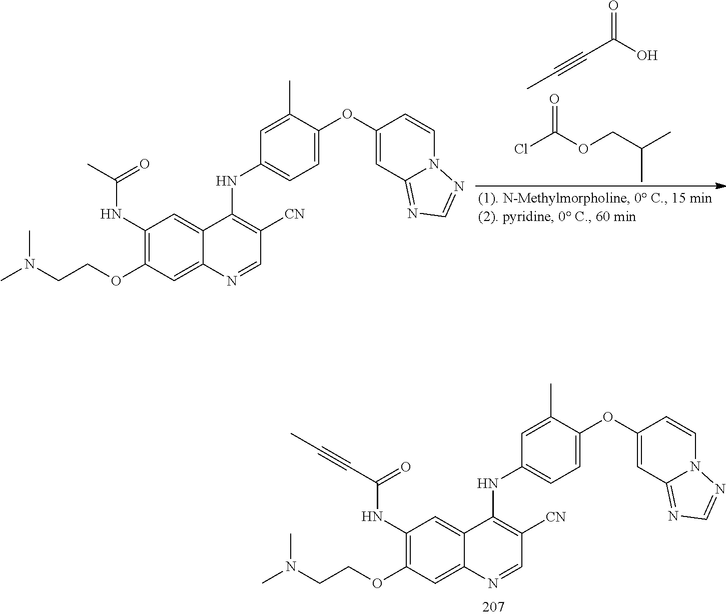

To a solution of but-2-ynoic acid (85.0 mg, 1.01 mmol) in THF (10.0 mL) was added isobutyl carbonochloridate (138.1 mg, 1.01 mmol) at 0° C. under N₂. The resulting mixture was stirred at 0° C. for 5 min. Then N-Methylmorpholine (204.5 mg, 2.02 mmol) was added to the mixture at 0° C. under N₂. The resulting mixture was stirred at 0° C. for 10 min. Then a solution of 4-((4-([1,2,4]triazolo[1,5-a]pyridin-7-yloxy)-3-methylphenyl)amino)-6-amino-7-(2-(dimethylamino)ethoxy)quinoline-3-carbonitrile (100.0 mg, 0.20 mmol) in pyridine (1.0 mL) was added dropwise to the mixture at 0° C. under N₂. The resulting mixture was stirred at 0° C. for 30 min. After the reaction was completed, the resulting mixture was quenched with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (5/1, v/v) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 31% to 41% in 8 min; 254 nm) to afford N-(4-((4-([1,2,4]triazolo[1,5-a]pyridin-7-yloxy)-3-methylphenyl)amino)-3-cyano-7-(2-(dimethylamino)ethoxy)quinolin-6-yl)but-2-ynamide (Compound 207) (19.1 mg, 16%) as a white solid. LCMS (ESI, m/z): [M+H]⁺=561.4. ¹H NMR (400 MHz, DMSO-d₆): δ 10.25 (s, 1H), 9.81 (s, 1H), 8.93 (d, J=7.6 Hz, 1H), 8.76 (s, 1H), 8.50 (s, 1H), 8.36 (s, 1H), 7.49 (s, 1H), 7.32-7.22 (m, 3H), 7.07-7.05 (m, 1H), 6.89 (s, 1H), 4.33-4.30 (m, 2H), 2.79-2.68 (m, 2H), 2.29 (s, 6H), 2.19-2.00 (m, 6H).

### Example S97. N-{7-ethoxy-4-[(3-methyl-4-{[1,2,4]triazolo[1,5-a]pyridin-7-yloxy}phenyl)amino]quinazolin-6-yl}-4-(4-methylpiperazin-1-yl)but-2-ynamide triformate (Compound 208)

### Step 1: Synthesis of 7-ethoxy-6-nitro-3H-quinazolin-4-one

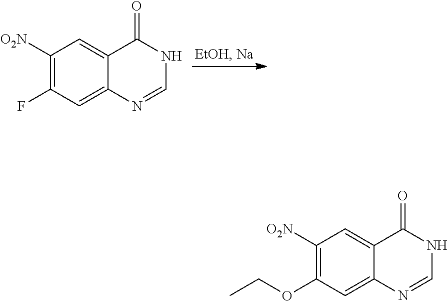 [SMILES: O=c1[nH]cnc2cc(F)c([N+](=O)[O-])cc12 ; CCOc1cc2nc[nH]c(=O)c2cc1[N+](=O)[O-]]

A solution of Na (824.4 mg, 35.86 mmol) in EtOH (50.0 mL) was stirred at room temperature for 30 min. To the above mixture was added 7-fluoro-6-nitro-3H-quinazolin-4-one (2.5 g, 11.95 mmol) at room temperature. The resulting mixture was stirred at 65° C. for 1.5 h. After the reaction was completed, the reaction mixture was quenched with water at room temperature. The resulting mixture was concentrated under vacuum. The residue was purified by flash column chromatography with CH₂Cl₂/CH₃OH (9/1, v/v) to afford 7-ethoxy-6-nitro-3H-quinazolin-4-one (2.5 g, 88%) as a yellow solid. LCMS (ESI, m/z): [M+H]⁺=236.1.

### Step 2: Synthesis of 4-chloro-7-ethoxy-6-nitroquinazoline

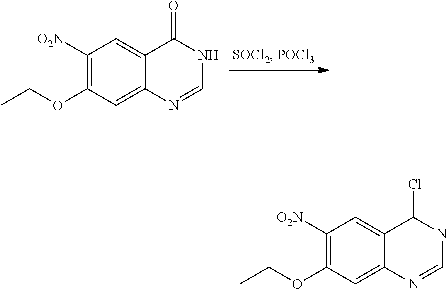 [SMILES: CCOc1cc2nc[nH]c(=O)c2cc1[N+](=O)[O-] ; CCOc1cc2c(cc1[N+](=O)[O-])C(Cl)NC=N2]

To a solution of 7-ethoxy-6-nitro-3H-quinazolin-4-one (1.0 g, 4.25 mmol) in SOCl₂ (15.0 mL) was added POCl₃ (5.0 mL) and DMF (0.1 mL) at room temperature. The resulting mixture was stirred at 85° C. for 1 h. After the reaction was completed, the mixture was evaporated in vacuo to afford 4-chloro-7-ethoxy-6-nitroquinazoline (1.0 g, crude) as a yellow solid. LCMS (ESI, m/z): [M+H]⁺=254.0.

### Step 3: Synthesis of 7-ethoxy-N-(3-methyl-4-{[1,2,4]triazolo[1,5-a]pyridin-7-yloxy}phenyl)-6-nitroquinazolin-4-amine

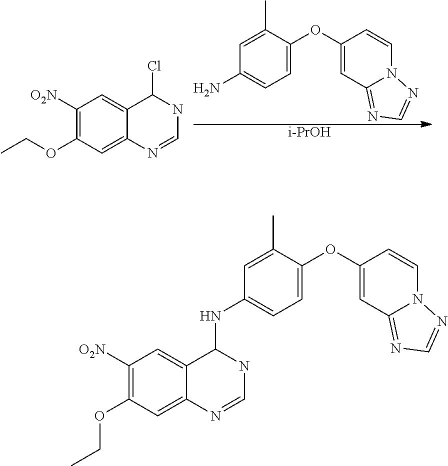

A mixture of 4-chloro-7-ethoxy-6-nitroquinazoline (1.0 g, crude) and 3-methyl-4-{[1,2,4]triazolo[1,5-a]pyridin-7-yloxy}aniline (1.0 g, 3.94 mmol) in i-PrOH (20.0 mL) was stirred at room temperature for 2 h. After the reaction was completed, the mixture was concentrated under reduced pressure. The residue was purified by flash column chromatography with CH₂Cl₂/MeOH (8/2, v/v) to afford 7-ethoxy-N-(3-methyl-4-{[1,2,4]triazolo[1,5-a]pyridin-7-yloxy}phenyl)-6-nitroquinazolin-4-amine (1.0 g, 55%) as a yellow solid. LCMS (ESI, m/z): [M+H]⁺=458.1.

### Step 4: Synthesis of 7-ethoxy-N4-(3-methyl-4-{[1,2,4]triazolo[1,5-a]pyridin-7-yloxy}phenyl)quinazoline-4,6-diamine

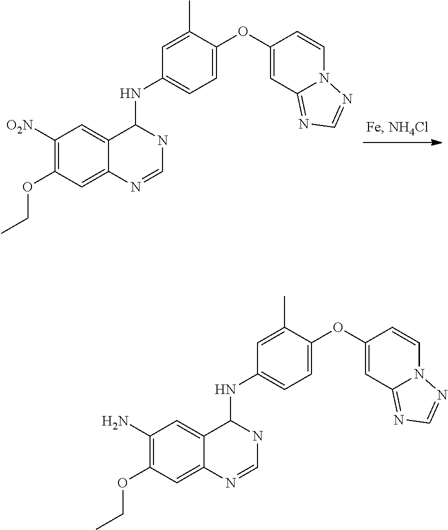 [SMILES: CCOc1cc2c(cc1[N+](=O)[O-])C(Nc1ccc(Oc3ccn4ncnc4c3)c(C)c1)NC=N2 ; CCOc1cc2c(cc1N)C(Nc1ccc(Oc3ccn4ncnc4c3)c(C)c1)NC=N2]

A mixture of N-(4-([1,2,4]triazolo[1,5-a]pyridin-7-yloxy)-3-methylphenyl)-7-ethoxy-6-nitroquinazolin-4-amine (1.0 g, 2.18 mmol), Fe (1.2 g, 21.86 mmol) and NH₄Cl (1.1 g, 21.86 mmol) in CH₃OH (20.0 mL)/H2O (2.0 mL) was heated at 80° C. for 2 h. After the reaction was completed, the resulting mixture was filtered. The filtrate was concentrated under reduced pressure. The residue was purified by flash column chromatography with CH₂Cl₂/CH₃OH (7/3, v/v)) to afford 7-ethoxy-N4-(3-methyl-4-{[1,2,4]triazolo[1,5-a]pyridin-7-yloxy}phenyl)quinazoline-4,6-diamine (600.0 mg, 64%) as a brown solid. LCMS (ESI, m/z): [M+H]+=428.2.

### Step 5: Synthesis of 1-methyl-4-(prop-2-yn-1-yl)piperazine

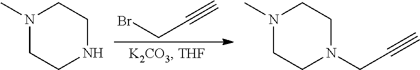

To a solution of 1-methylpiperazine (10.0 g, 99.83 mmol) in THF (30.0 mL) was added propargyl bromide (11.8 g, 99.83 mmol) and K₂CO₃ (27.6 g, 199.6 mmol) at room temperature. The mixture was stirred at 65° C. for 16 h. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (9/1, v/v) to afford 1-methyl-4-(prop-2-yn-1-yl)piperazine (3.0 g, 21%) as a brown oil. LCMS (ESI, m/z): [M+H]+=139.1.

### Step 6: Synthesis of 4-(4-methylpiperazin-1-yl)but-2-ynoic acid

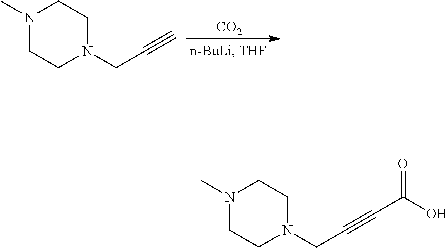

To a solution of 1-methyl-4-(prop-2-yn-1-yl)piperazine (2.0 g, 14.5 mmol) in THF (30.0 mL) was added dropwise n-BuLi (11.6 mL, 2.5 mol/L) at −78° C. under N₂. The mixture was stirred at −78° C. for 1.5 h. Then CO₂ (g) was inserted into the mixture at −78° C. The mixture was stirred at −78° C. for 1 h. After the reaction was completed, the reaction mixture was quenched with H₂O and extracted with EtOAc. The aqueous layer was evaporated under reduced pressure to give the crude product. The crude product was dissolved in methanol and then filtered. The filtrate was concentrated under vacuum to afford 4-(4-methylpiperazin-1-yl)but-2-ynoic acid (2.4 g, crude) as a brown solid. LCMS (ESI, m/z): [M+H]+=183.1.

### Step 7: Synthesis of N-{7-ethoxy-4-[(3-methyl-4-{[1,2,4]triazolo[1,5-a]pyridin-7-yloxy}phenyl)amino]quinazolin-6-yl}-4-(4-methylpiperazin-1-yl)but-2-ynamide triformate (Compound 208)

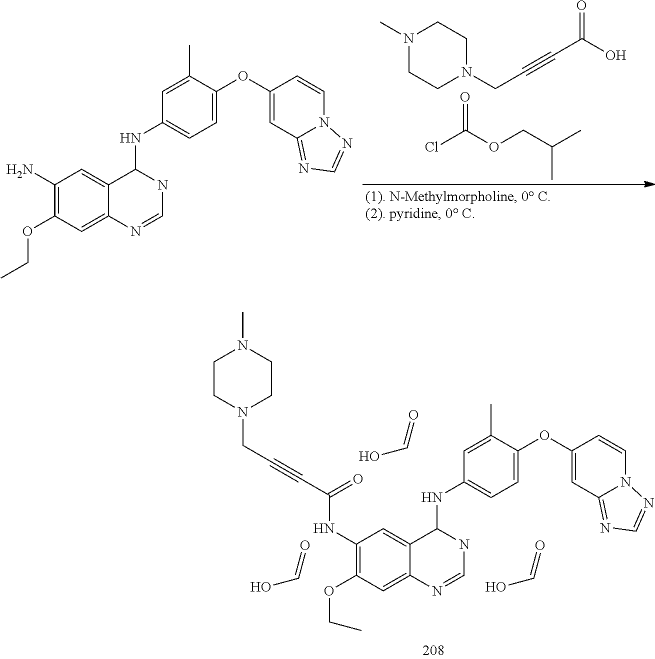

To a solution of 4-(4-methylpiperazin-1-yl)but-2-ynoic acid (1.0 g, crude) and in THF (10.0 mL) was added 2-methylpropyl chloroformate (575.1 mg, 4.21 mmol) at 0° C. The mixture was stirred at 0° C. for 5 min. To the above mixture was added N-Methylmorpholine (851.8 mg, 8.42 mmol) at 0° C. The resulting mixture was stirred at 0° C. for additional 10 min. Then a solution of 7-ethoxy-N4-(3-methyl-4-{[1,2,4]triazolo[1,5-a]pyridin-7-yloxy}phenyl)quinazoline-4,6-diamine (300.0 mg, 0.70 mmol) in pyridine (3.0 mL) was added dropwise to the mixture at 0° C. The mixture was stirred at 0° C. for additional 40 min. After the reaction was completed, the reaction mixture was quenched with water and extracted with ethyl acetate. The combined organic layers were washed with brine, dried over anhydrous Na₂SO₄ and filtered. The filtrate was concentrated under reduced pressure. The residue was purified by reverse phase flash column chromatography with CH₃CN/H₂O (5/5, v/v) and then purified by Prep-HPLC with the following conditions Column (Xselect CSH F-Phenyl OBD column, 19×250 mm, 5 μm; Mobile Phase A: Water (0.1% FA), Mobile Phase B: MeOH-HPLC; Flow rate: 25 mL/min; Gradient: 30% B to 30% B in 12 min; Wavelength: 254 nm) to afford N-{7-ethoxy-4-[(3-methyl-4-{[1,2,4]triazolo[1,5-a]pyridin-7-yloxy}phenyl)amino]quinazolin-6-yl}-4-(4-methylpiperazin-1-yl)but-2-ynamide triformate (Compound 208) (9.4 mg, 2%) as a yellow solid. LCMS (ESI, m/z): [M+H]⁺=592.2. ¹H NMR (400 MHz, CD₃OD): δ 9.07 (s, 1H), 8.76 (d, J=7.2 Hz, 1H), 8.49 (s, 3H), 8.30 (s, 1H), 7.76-7.70 (m, 2H), 7.27-7.19 (m, 2H), 7.12-7.09 (m, 2H), 6.86 (d, J=2.4 Hz, 1H), 4.47 (s, 2H), 4.43-4.37 (m, 2H), 3.96-3.82 (m, 4H), 3.45 (s, 3H), 2.27 (s, 3H), 1.63-1.55 (m, 3H).

### Example S98. N-{7-[3-(dimethylamino)propoxy]-5-fluoro-4-[(3-methyl-4-{[1,2,4]triazolo[1,5-a]pyridin-7-ylmethyl}phenyl)amino]quinazolin-6-yl}but-2-ynamide (Compound 209)

### Step 1: Synthesis of 4-[3-(dimethylamino)propoxy]-2,6-difluorobenzonitrile

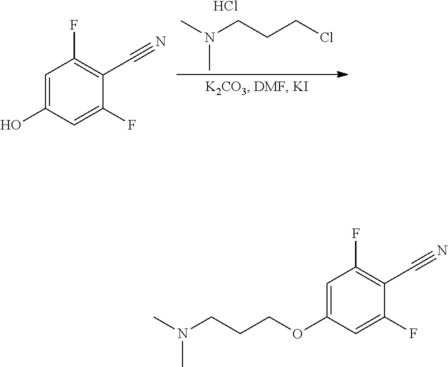

To a solution of 2,6-difluoro-4-hydroxybenzonitrile (20.0 g, 128.95 mmol) in DMF (600.0 mL) was added (3-chloropropyl)dimethylamine (31.4 g, 257.89 mmol), K₂CO₃ (53.5 g, 386.84 mmol) and KI (4.3 g, 25.79 mmol) at room temperature. The resulting mixture was stirred at 50° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford 4-[3-(dimethylamino)propoxy]-2,6-difluorobenzonitrile (10.0 g, 32%) as a white solid. LCMS (ESI, m/z): [M+H]⁺=241.1.

### Step 2: Synthesis of 2-amino-4-[3-(dimethylamino)propoxy]-6-fluorobenzonitrile

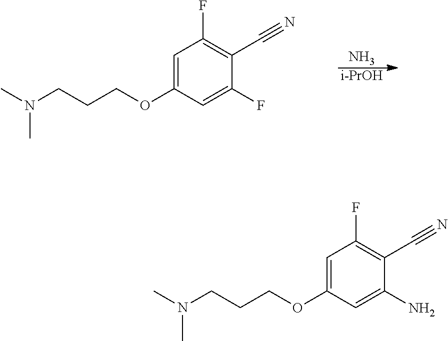

To a solution of 4-[3-(dimethylamino)propoxy]-2,6-difluorobenzonitrile (10.0 g, 41.62 mmol) in i-PrOH (50.0 mL) was added ammonia (28.4 g, 25%) at room temperature. The resulting mixture was stirred at 100° C. for 16 h. After the reaction was completed, the resulting mixture was concentrated under reduced pressure. The residue was purified by flash column chromatography with CH₂Cl₂/MeOH (5/1, v/v) to afford 2-amino-4-[3-(dimethylamino)propoxy]-6-fluorobenzonitrile (8.0 g, 81%) as an off-white solid. LCMS (ESI, m/z): [M+H]⁺=238.1.

### Step 3: Synthesis of 6-amino-4-[3-(dimethylamino)propoxy]-2-fluoro-3-iodobenzonitrile

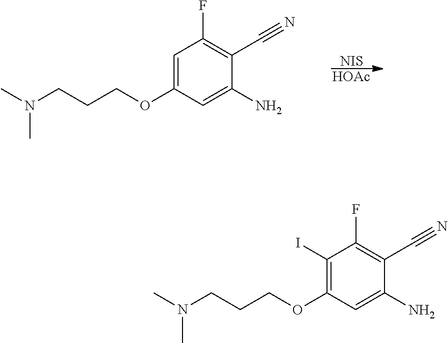

To a solution of 2-amino-4-[3-(dimethylamino)propoxy]-6-fluorobenzonitrile (4.0 g, 16.89 mmol) in AcOH (100.0 mL) was added NIS (3.8 g, 16.89 mmol) at room temperature. The resulting mixture was stirred at 50° C. for 16 h. After the reaction was completed, the resulting mixture was concentrated under reduced pressure. The residue was purified by flash column chromatography with CH₂Cl₂/MeOH (5/1, v/v) to afford 6-amino-4-[3-(dimethylamino)propoxy]-2-fluoro-3-iodobenzonitrile (3.9 g, 63%) as a yellow solid. LCMS (ESI, m/z): [M+H]⁺=364.0.

### Step 4: Synthesis of (E)-N′-{2-cyano-5-[3-(dimethylamino)propoxy]-3-fluoro-4-iodophenyl}-N,N-dimethylmethanimidamide

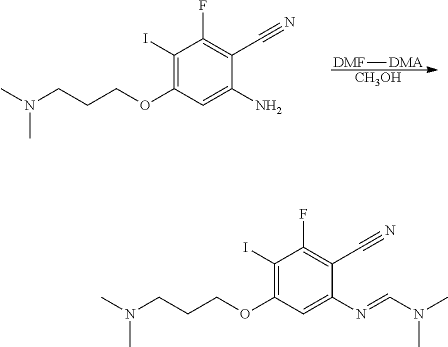

To a solution of 6-amino-4-[3-(dimethylamino)propoxy]-2-fluoro-3-iodobenzonitrile (3.9 g, 10.74 mmol) in CH₃OH (65.0 mL) was added DMF-DMA (1.9 g, 16.12 mmol) at room temperature. The resulting mixture was stirred at 75° C. for 2 h. After the reaction was completed, the mixture was concentrated under vacuum to afford (E)-N′-{2-cyano-5-[3-(dimethylamino)propoxy]-3-fluoro-4-iodophenyl}-N,N-dimethylmethanimidamide (4.3 g, crude) as a yellow solid. LCMS (ESI, m/z): [M+H]⁺=419.1.

### Step 5: Synthesis of 7-[3-(dimethylamino)propoxy]-5-fluoro-6-iodo-N-(3-methyl-4-{[1,2,4]triazolo[1,5-a]pyridin-7-ylmethyl}phenyl)quinazolin-4-amine

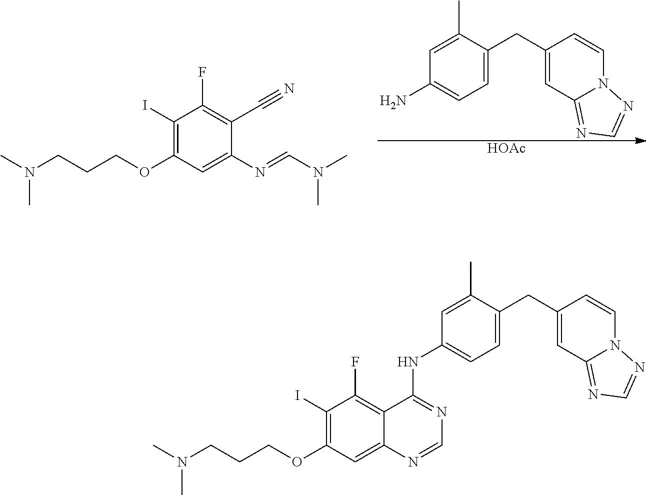

To a solution of (E)-N′-{2-cyano-5-[3-(dimethylamino)propoxy]-3-fluoro-4-iodophenyl}-N,N-dimethylmethanimidamide (4.3 g, crude) in AcOH (65.0 mL) was added 3-methyl-4-{[1,2,4]triazolo[1,5-a]pyridin-7-ylmethyl}aniline (2.4 g, 10.28 mmol) at room temperature. The resulting mixture was stirred at 85° C. for 2 h. After the reaction was completed, the resulting mixture was concentrated under reduced pressure. The residue was purified by flash column chromatography with CH₂Cl₂/MeOH (5/1, v/v) to afford 7-[3-(dimethylamino)propoxy]-5-fluoro-6-iodo-N-(3-methyl-4-{[1,2,4]triazolo[1,5-a]pyridin-7-ylmethyl}phenyl)quinazolin-4-amine (3.0 g, 47%) as a white solid. LCMS (ESI, m/z): [M+H]⁺=612.1.

### Step 6: Synthesis of 7-[3-(dimethylamino)propoxy]-N6-(diphenylmethylidene)-5-fluoro-N4-(3-methyl-4-{[1,2,4]triazolo[1,5-a]pyridin-7-ylmethyl}phenyl)quinazoline-4,6-diamine

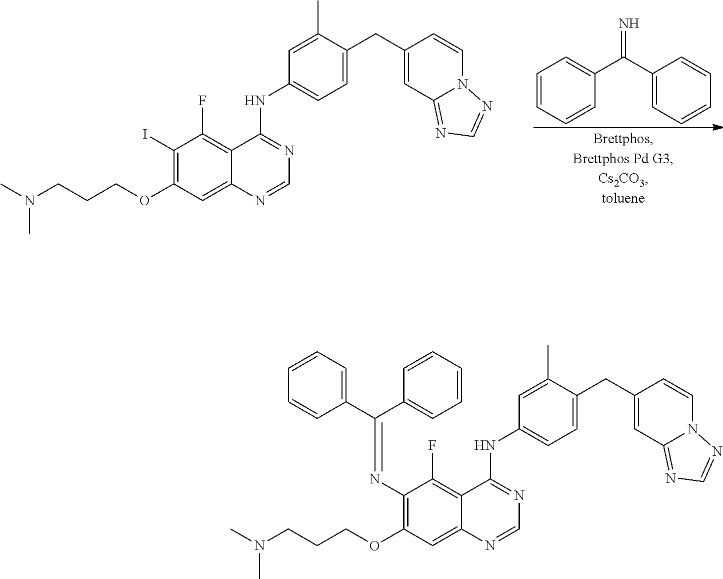 [SMILES: Cc1cc(Nc2ncnc3cc(OCCCN(C)C)c(I)c(F)c23)ccc1Cc1ccn2ncnc2c1 ; N=C(c1ccccc1)c1ccccc1 ; Cc1cc(Nc2ncnc3cc(OCCCN(C)C)c(N=C(c4ccccc4)c4ccccc4)c(F)c23)ccc1Cc1ccn2ncnc2c1]

To a solution of 7-[3-(dimethylamino)propoxy]-5-fluoro-6-iodo-N-(3-methyl-4-{[1,2,4]triazolo[1,5-a]pyridin-7-ylmethyl}phenyl)quinazolin-4-amine (1.2 g, 1.96 mmol) in toluene (30.0 mL) was added diphenylmethanimine (1.1 g, 5.89 mmol), Cs₂CO₃ (1.9 g, 5.89 mmol), BrettPhos (0.2 g, 0.39 mmol) and BrettPhos Pd G3 (0.2 g, 0.20 mmol) at room temperature under N₂. The resulting mixture was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (5/1, v/v) to afford 7-[3-(dimethylamino)propoxy]-N6-(diphenylmethylidene)-5-fluoro-N4-(3-methyl-4-{[1,2,4]triazolo[1,5-a]pyridin-7-ylmethyl}phenyl)quinazoline-4,6-diamine (600.0 mg, 46%) as a yellow solid. LCMS (ESI, m/z): [M+H]⁺=665.3.

### Step 7: Synthesis of 7-[3-(dimethylamino)propoxy]-5-fluoro-N4-(3-methyl-4-{[1,2,4]triazolo[1,5-a]pyridin-7-ylmethyl}phenyl)quinazoline-4,6-diamine

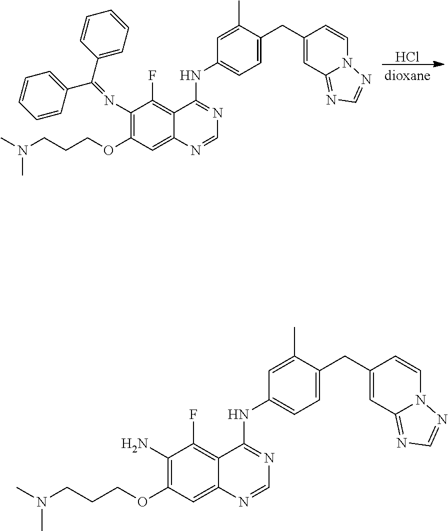 [SMILES: Cc1cc(Nc2ncnc3cc(OCCCN(C)C)c(N=C(c4ccccc4)c4ccccc4)c(F)c23)ccc1Cc1ccn2ncnc2c1 ; Cl ; Cc1cc(Nc2ncnc3cc(OCCCN(C)C)c(N)c(F)c23)ccc1Cc1ccn2ncnc2c1]

The solution of 7-[3-(dimethylamino)propoxy]-N6-(diphenylmethylidene)-5-fluoro-N4-(3-methyl-4-{[1,2,4]triazolo[1,5-a]pyridin-7-ylmethyl}phenyl)quinazoline-4,6-diamine (270.0 mg, 0.41 mmol) in HCl/dioxane (20.0 mL, 4 mol/L) was stirred at room temperature for 2 h. After the reaction was completed, the mixture was basified to pH=8 with saturated NaHCO₃(aq.). The resulting mixture was extracted with DCM. The combined organic layer was washed with brine, dried over anhydrous sodium sulfate and filtered. The filtrate was concentrated under reduced pressure. The residue was purified by flash column chromatography with CH₂Cl₂/MeOH (5/1, v/v) to afford 7-[3-(dimethylamino)propoxy]-5-fluoro-N4-(3-methyl-4-{[1,2,4]triazolo[1,5-a]pyridin-7-ylmethyl}phenyl)quinazoline-4,6-diamine (220.0 mg, 72%) as a yellow solid. LCMS (ESI, m/z): [M+H]⁺=501.2.

### Step 8: Synthesis of N-{7-[3-(dimethylamino)propoxy]-5-fluoro-4-[(3-methyl-4-{[1,2,4]triazolo[1,5-a]pyridin-7-ylmethyl}phenyl)amino]quinazolin-6-yl}but-2-ynamide (Compound 209)

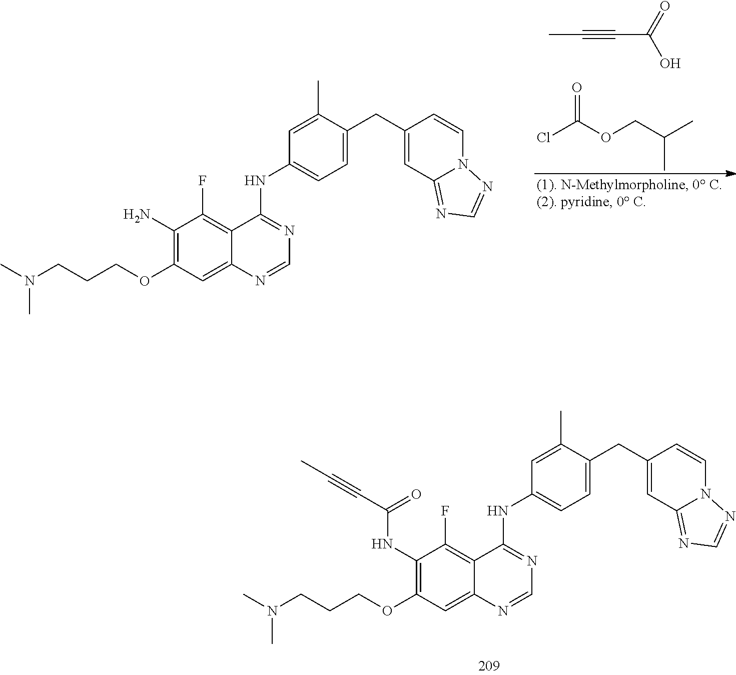

To a solution of 2-butynoic acid (40.3 mg, 0.48 mmol) in THF (5.0 mL) was added 2-methylpropyl carbonochloridate (65.5 mg, 0.48 mmol) at 0° C. The resulting mixture was stirred at 0° C. for 5 min. Then N-Methylmorpholine (97.0 mg, 0.96 mmol) was added to the mixture at 0° C. The resulting mixture was stirred at 0° C. for 10 min. Then a solution of 7-[3-(dimethylamino)propoxy]-5-fluoro-N4-(3-methyl-4-{[1,2,4]triazolo[1,5-a]pyridin-7-ylmethyl}phenyl)quinazoline-4,6-diamine (60.0 mg, 0.12 mmol) in pyridine (1.0 mL) was added dropwise to the mixture at 0° C. The resulting mixture was stirred at 0° C. for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 52% B to 61% B in 8 min; Wavelength: 254 nm) to afford N-{7-[3-(dimethylamino)propoxy]-5-fluoro-4-[(3-methyl-4-{[1,2,4]triazolo[1,5-a]pyridin-7-ylmethyl}phenyl)amino]quinazolin-6-yl}but-2-ynamide (Compound 209) (6.2 mg, 9%) as a white solid. LCMS (ESI, m/z): [M+H]⁺=567.2.41 NMR (400 MHz, DMSO-d₆): δ 10.13 (s, 1H), 8.97-8.94 (m, 1H), 8.87-8.85 (m, 1H), 8.48-8.44 (m, 2H), 7.57-7.52 (m, 3H), 7.25 (d, J=8.4 Hz, 1H), 7.11 (s, 1H), 7.03-7.01 (m, 1H), 4.22-4.19 (m, 2H), 4.13 (s, 2H), 2.41-2.37 (m, 2H), 2.25 (s, 3H), 2.16 (s, 6H), 2.07 (s, 3H), 1.90-1.87 (m, 2H).

### Example S99. N-{5-fluoro-4-[(2-fluoro-5-methyl-4-{[1,2,4]triazolo[1,5-a]pyridin-7-yloxy}phenyl)amino]quinazolin-6-yl}but-2-ynamide (Compound 210)

### Step 1: Synthesis of N-{5-fluoro-4-[(2-fluoro-5-methyl-4-{[1,2,4]triazolo[1,5-a]pyridin-7-yloxy}phenyl)amino]quinazolin-6-yl}but-2-ynamide (Compound 210)

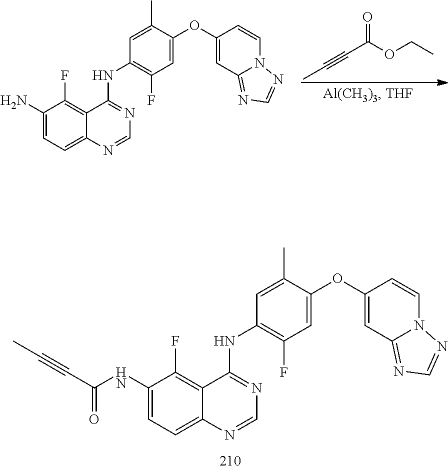

To a mixture of 5-fluoro-N4-(2-fluoro-5-methyl-4-{[1,2,4]triazolo[1,5-a]pyridin-7-yloxy}phenyl)quinazoline-4,6-diamine (130.0 mg, 0.31 mmol) and but-2-ynoic acid ethyl ester (104.3 mg, 0.93 mmol) in THF (8.0 mL) was added Al(CH₃)₃ (0.6 mL, 2 mol/L) at 0° C. The resulting mixture was stirred at 80° C. for 3 h. After the reaction was completed, the reaction was quenched with NH₄Cl (aq) at 0° C. and then extracted with DCM. The combined organic layer was washed with brine, dried over anhydrous sodium sulfate and filtered. The filtrate was concentrated under reduced pressure. The residue was purified by flash column chromatography, eluted with CH₂Cl₂/MeOH (10/1, v/v) to afford N-{5-fluoro-4-[(2-fluoro-5-methyl-4-{[1,2,4]triazolo[1,5-a]pyridin-7-yloxy}phenyl)amino]quinazolin-6-yl}but-2-ynamide (Compound 210) (63.6 mg, 41%) as an off-white solid. LCMS (ESI, m/z): [M+H]⁺=486.1. ¹H NMR (400 MHz, DMSO-d₆): δ 10.76 (s, 1H), 9.18-9.15 (m, 1H), 8.99 (d, J=7.6 Hz, 1H), 8.55 (s, 1H), 8.42 (s, 1H), 8.05-8.01 (m, 1H), 7.82 (d, J=8.8 Hz, 1H), 7.66 (d, J=9.2 Hz, 1H), 7.32 (d, J=10.4 Hz, 1H), 7.08-7.05 (m, 1H), 6.90 (d, J=2.8 Hz, 1H), 2.19 (s, 3H), 2.09 (s, 3H).

### Example S100. N-(4-{[3-methyl-4-(quinoxalin-6-yloxy)phenyl}amino]quinazolin-6-yl)but-2-ynamide (Compound 211)

### Step 1: Synthesis of 6-(2-methyl-4-nitrophenoxy)quinoxaline

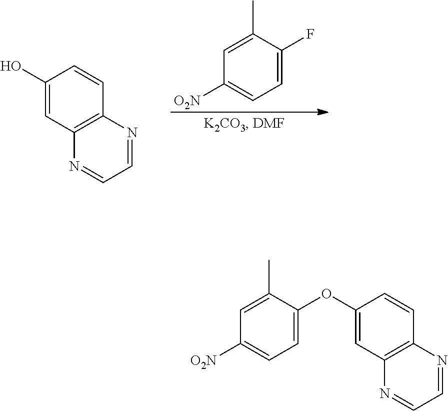 [SMILES: Oc1ccc2nccnc2c1 ; Cc1cc([N+](=O)[O-])ccc1F ; Cc1cc([N+](=O)[O-])ccc1Oc1ccc2nccnc2c1]

To a solution of quinoxalin-6-ol (2.0 g, 13.69 mmol) in DMF (20.0 mL) was added 1-fluoro-2-methyl-4-nitrobenzene (2.1 g, 13.69 mmol) and K₂CO₃ (5.7 g, 41.06 mmol) at room temperature. The resulting mixture was stirred at 50° C. for 16 h. After the reaction was completed, the resulting mixture was diluted with water and extracted with dichloromethane. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 6-(2-methyl-4-nitrophenoxy)quinoxaline (3.8 g, 97%) as a yellow solid. LCMS (ESI, m/z): [M+H]⁺=282.1.

### Step 2: Synthesis of 3-methyl-4-(quinoxalin-6-yloxy)aniline

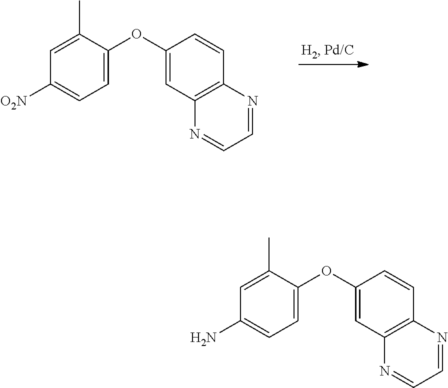 [SMILES: Cc1cc([N+](=O)[O-])ccc1Oc1ccc2nccnc2c1 ; Cc1cc(N)ccc1Oc1ccc2nccnc2c1]

To a solution of 6-(2-methyl-4-nitrophenoxy)quinoxaline (1.5 g, 5.33 mmol) in EtOH/H₂O (20.0 mL/4.0 mL) was added NH₄Cl (1.4 g, 26.67 mmol) and Fe (1.5 g, 26.67 mmol) at room temperature. The resulting mixture was stirred at 80° C. for 2 h. After the reaction was completed, the resulting mixture was filtered. The filtrate was concentrated under reduced pressure. The residue was purified by reverse phase flash column chromatography with acetonitrile/water (1/1, v/v) to afford 3-methyl-4-(quinoxalin-6-yloxy)aniline (895.0 mg, 66%) as a yellow solid. LCMS (ESI, m/z): [M+H]⁺=2521

### Step 3: Synthesis of N-(4-{[3-methyl-4-(quinoxalin-6-yloxy)phenyl]amino}quinazolin-6-yl)but-2-ynamide (Compound 211)

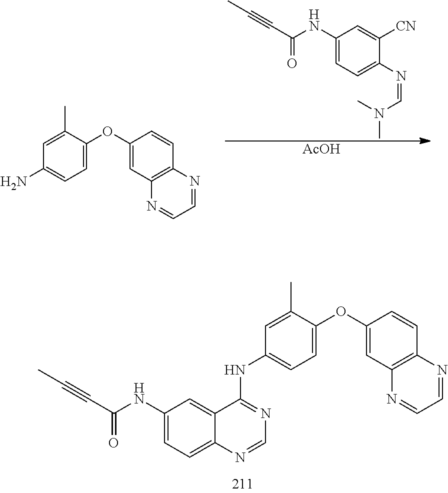 [SMILES: "AcOH" ; Cc1cc(N)ccc1Oc1ccc2nccnc2c1 ; CC#CC(=O)Nc1ccc(/N=C\N(C)C)c(C#N)c1 ; CC#CC(=O)Nc1ccc2ncnc(Nc3ccc(Oc4ccc5nccnc5c4)c(C)c3)c2c1]

To a solution of 3-methyl-4-(quinoxalin-6-yloxy)aniline (300.0 mg, 1.19 mmol) in AcOH (10.0 mL) was added N-{3-cyano-4-[(Z)-[(dimethylamino)methylidene]amino]phenyl}but-2-ynamide (303.6 mg, 1.19 mmol at room temperature. The resulting mixture was stirred at 85° C. for 4 h. After the reaction was completed, the resulting mixture was concentrated under reduced pressure. The residue was purified by Prep-HPLC (Column: Xselect CSH C18 OBD Column 30×150 mm, 5 μm; Mobile Phase A: Water (0.1% FA), Mobile Phase B: ACN; Flow rate: 60 mL/min; Gradient: 20% B to 33% B in 8 min, Wave Length: 254 nm) to afford N-(4-{[3-methyl-4-(quinoxalin-6-yloxy)phenyl]amino}quinazolin-6-yl)but-2-ynamide (Compound 211) (58.7 mg, 10%) as a yellow solid. LCMS (ESI, m/z): [M+H]⁺=461.3. ¹H NMR (400 MHz, DMSO-d₆): δ 10.96 (s, 1H), 9.90 (s, 1H), 8.87-8.78 (m, 3H), 8.55 (s, 1H), 8.16-8.14 (m, 1H), 7.95-7.55 (m, 5H), 7.27-7.13 (m, 2H), 2.20 (s, 3H), 2.10 (s, 3H).

### Example S101. N-[4-({3-methyl-4-[(1-methyl-1,3-benzodiazol-5-yl)oxy]phenyl}amino)quinazolin-6-yl]but-2-ynamide (Compound 212)

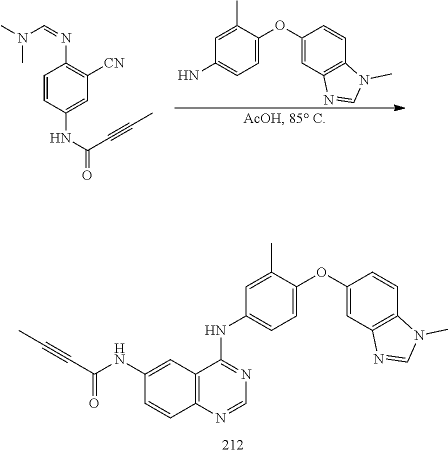 [SMILES: CC#CC(=O)Nc1ccc(/N=C\N(C)C)c(C#N)c1 ; Cc1cc(N)ccc1Oc1ccc2c(c1)ncn2C ; CC#CC(=O)Nc1ccc2ncnc(Nc3ccc(Oc4ccc5c(c4)ncn5C)c(C)c3)c2c1]

A mixture of N-{3-cyano-4-[(Z)-[(dimethylamino)methylidene]amino]phenyl}but-2-ynamide (120.0 mg, 0.47 mmol) and 3-methyl-4-[(1-methyl-1,3-benzodiazol-5-yl)oxy]aniline (119.5 mg, 0.47 mmol) in AcOH (3.0 mL) was stirred at 85° C. for 2 h. After the reaction was completed, the resulting mixture was concentrated under reduced pressure. The residue was purified by flash column chromatography with CH₂Cl₂/CH₃OH (9/1, v/v) and then purified by Prep-HPLC with the following conditions (Column: Xselect CSH C₁₈ OBD Column 30×150 mm, 5 μm; Mobile Phase A: Water (0.1% FA), Mobile Phase B: ACN; Flow rate: 60 mL/min; Gradient: 6% B to 20% B in 12 min; Wave Length: 254 nm) to afford N-[4-({3-methyl-4-[(1-methyl-1,3-benzodiazol-5-yl)oxy]phenyl}amino)quinazolin-6-yl]but-2-ynamide (Compound 212) (28.5 mg, 13%) as a yellow solid. LCMS (ESI, m/z): [M+H]⁺=463.2. ¹H NMR (400 MHz, DMSO-d₆): δ 10.90 (s, 1H), 9.77 (s, 1H), 8.73 (s, 1H), 8.50 (s, 1H), 8.17 (s, 1H), 7.80-7.70 (m, 3H), 7.58-7.56 (m, 2H), 7.10 (d, J=2.0 Hz, 1H), 7.02-6.99 (m, 1H), 6.86 (d, J=8.8 Hz, 1H), 3.84 (s, 3H), 2.25 (s, 3H), 2.09 (s, 3H).

### Example S102. N-[5-fluoro-4-({3-methyl-4-[(1-methyl-1,3-benzodiazol-5-yl)oxy]phenyl}amino)quinazolin-6-yl]but-2-ynamide (Compound 213)

### Step 1: Synthesis of N-{3-cyano-4-[(E)-[(dimethylamino)methylidene]amino]-2-fluorophenyl}but-2-ynamide

To a stirred mixture of (E)-N′-(4-amino-2-cyano-3-fluorophenyl)-N,N-dimethylmethanimidamide (500.0 mg, 2.43 mmol) and 2-butynoic acid (407.7 mg, 4.85 mmol) in pyridine (12.0 mL) was added EDCI (929.6 mg, 4.85 mmol) at room temperature. The resulting mixture was stirred at room temperature for 30 min. After the reaction was completed, the resulting mixture was concentrated under reduced pressure. The residue was purified by reverse phase flash column chromatography with acetonitrile/water (64/36, v/v) to afford N-{3-cyano-4-[(E)-[(dimethylamino)methylidene]amino]-2-fluorophenyl}but-2-ynamide (600.0 mg, 90%) as a brown oil. LCMS (ESI, m/z): [M+H]⁺=273.1.

### Step 2: Synthesis of N-[5-fluoro-4-({3-methyl-4-[(1-methyl-1,3-benzodiazol-5-yl)oxy]phenyl}amino)quinazolin-6-yl]but-2-ynamide (Compound 213)

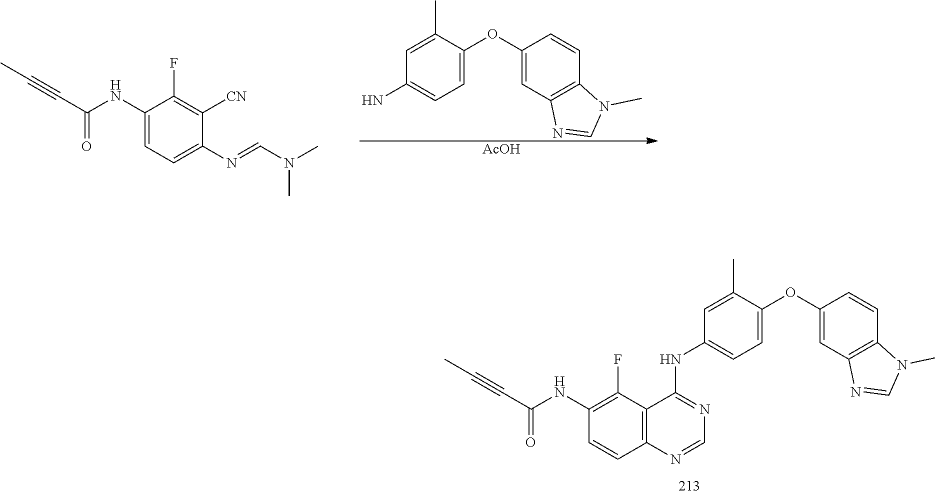 [SMILES: "AcOH" ; CC#CC(=O)Nc1ccc(/N=C/N(C)C)c(C#N)c1F ; Cc1cc(N)ccc1Oc1ccc2c(c1)ncn2C ; CC#CC(=O)Nc1ccc2ncnc(Nc3ccc(Oc4ccc5c(c4)ncn5C)c(C)c3)c2c1F]

To a stirred mixture of 3-methyl-4-[(1-methyl-1,3-benzodiazol-5-yl)oxy]aniline (180.0 mg, 0.71 mmol) in AcOH (6.0 mL) was added N-{3-cyano-4-[(E)-[(dimethylamino)methylidene]amino]-2-fluorophenyl}but-2-ynamide (387.0 mg, 1.42 mmol) at room temperature. The resulting mixture was stirred at 80° C. for 2 h. After the reaction was completed, the resulting mixture was concentrated under reduced pressure. The residue was purified by reverse phase flash column chromatography with acetonitrile/water (64/36, v/v) and then purified by Prep-HPLC with the following conditions (Column: XBridge Prep Phenyl OBD Column, 19×250 mm, 5 μm; Mobile Phase A: Water (10 mmol/L NH₄HCO₃), Mobile Phase B: MeOH-HPLC; Flow rate: 25 mL/min; Gradient: 68% B to 68% B in 10 min; Wave Length: 254 nm) to afford N-[5-fluoro-4-({3-methyl-4-[(1-methyl-1,3-benzodiazol-5-yl)oxy]phenyl}amino)quinazolin-6-yl]but-2-ynamide (9.6 mg, 2%) as a white solid. LCMS (ESI, m/z): [M+H]⁺=481.1. ¹H NMR (400 MHz, DMSO-d₆): δ 10.69 (s, 1H), 9.13-9.04 (m, 1H), 8.52 (s, 1H), 8.18 (s, 1H), 8.05-7.95 (m, 1H), 7.63-7.57 (m, 3H), 7.52-7.49 (m, 1H), 7.12 (d, J=2.0 Hz, 1H), 7.02-7.00 (m, 1H), 6.85 (d, J=8.8 Hz, 1H), 3.85 (s, 3H), 2.25 (s, 3H), 2.09 (s, 3H).

### BIOLOGICAL EXAMPLES

### Example B1. Reaction Biology Kinase Activity Assay

Inhibition of kinase activity was assessed using a kinase activity assay (Anastassiadis, T. et al. Nat Biotechnol 2011 29: 1039-1045). In particular, substrate (poly Glu-Tyr sodium salt (Sigma Aldrich, cat #P7244)) was freshly prepared in reaction buffer (20 mM Hepes (pH 7.5), 10 mM MgCl₂, 1 mM EGTA, 0.02% Brij35, 0.02 mg/ml BSA, 0.1 mM Na₃VO₄, 2 mM DTT, 1% DMSO). EGFR, ERBB2 or ERBB2 mutant kinases were added to the substrate solution and mixed at the final concentrations in Table B1, below. Compounds were dissolved in 100% DMSO and the resulting solutions were added to the kinase reaction mixture. The kinase reaction mixture was incubated with the compounds for 20 minutes at room temperature. 100 μM ³³P-ATP (Specific activity 10 μCi/μl) was then added to initiate the reaction. This mixture was incubated for 2 hours at 25° C.

Reactions were spotted onto P81 ion exchange filter paper (Whatman). Unbound phosphate was removed by extensive washing of filters in 0.75% phosphoric acid. Radioactivity was detected using a HotSpot, a proprietary technology of Reaction Biology Corporation (Anastassiadis, T. et al., Nat Biotechnol. 2011 29(11):1039-45). Kinase activity data were expressed as the percentage kinase activity remaining in test samples relative to vehicle only (i.e., DMSO only) control reactions. IC₅₀ values and curve fits were obtained using Prism (GraphPad Software), using a nonlinear regression model with a sigmoidal dose response and are shown in Table B1.

TABLE B1

| | Final enzyme | Tucatinib | Compound 1 |
|---|---|---|---|
| Kinase | concentration (nM) | IC₅₀ (nM) | IC₅₀ (nM) |
| ERBB2/HER2 | 50 | 3.07 | 0.49 |
| ERBB2 (D769H) | 150 | 5 | 1.04 |
| ERBB2 (D769Y) | 15 | 13.75 | 1.56 |
| ERBB2 (P1170A) | 20 | 3.99 | 0.68 |
| ERBB2 (R896C) | 200 | 2.95 | 2.29 |
| ERBB2 | 100 | 4.43 | 0.97 |
| (V777_G778insCG) | | | |
| ERBB2 (V777L) | 40 | 4.3 | 1.38 |
| EGFR | 3 | 954 | 58 |

### Example B2. Cell Viability Assays

Cells were treated with compounds, and cell viability was measured as a metric of kinase inhibition.

BT-474, A431, MDA-MB-175VII, NCI-H1781, MCF7, and Ba/F3 cell lines were tested. The Ba/F3 cell line is IL-3-dependent mouse cell line derived from the C3H mouse strain. Ba/F3 cell lines were engineered to express human ERBB2 or EGFR kinases, rendering the cells IL-3 independent. The lines were generated via retroviral transduction utilizing a Moloney murine leukemia virus (MMLV) promoter, and constructs are stably integrated into the cell genome. The sequences of the ERBB2 and EGFR genes used were NCBI Reference Sequences NM 004448.3 and NM 005228.3, respectively.

BT-474, A431, MDA-MB-175VII, NCI-H1781 and MCF7 cells were grown in the appropriate growth medium as described in Table B2 below, and harvested at 50-80% confluence. BT-474, A431, MDA-MB-175VII, NCI-H1781 and MCF7 cells were counted and seeded at 2,000 or 1,500 cells per well in 384-well tissue culture plates (see Table B2). Similarly, Ba/F3 cell lines engineered to express EGFR, ERBB2, or ERBB2 mutants were grown, harvested, counted and seeded at 3000 cells per well in 96-well plates for the data sets shown in Tables B3-B4, and seeded at 300 cells per well in 384-well plates for the data set shown in Table B5. A subset of wells contained media only (low control, “LC”).

Table B2 provides the growth media and number of cells seeded per well for the each cell line.

TABLE B2

| | | Number of cells |
|---|---|---|
| Cell Line | Growth Medium | seeded per well |
| BT-474 | Dulbecco's Modified Eagle | 2000 |
| | Medium (DMEM) + 10% | |
| | fetal bovine serum (FBS) | |
| A431 | DMEM + 10% FBS | 1500 |
| MDA-MB-175VII | RPMI + 10% FBS | 2000 |
| NCI-H1781 | RPMI + 10% FBS | 1500 |
| MCF7 | EMEM + 10% FBS + | 2000 |
| | 100 ng/ml human insulin | |
| Ba/F3 | RPMI + 10% FBS | 3000 or 300* |

*Ba/F3 cells in Tables B3-B4 were seeded at 3000 cells per well in 96-well plates; Ba/F3 cells in Table B5 were seeded at 300 cells per well in 384-well plates.

Compounds were dissolved in DMSO and serially diluted. Serially-diluted compound or a DMSO only control (high control, “HC”) was added to the plated cells in each well. Compounds were tested at concentrations of about 10 μM to 0.51 nM, using three-fold dilutions. The final proportion of DMSO never exceeded 0.1%.

Plates were placed in a 37° C., 5% CO₂ incubator for 72 hours. Plates were then removed from the incubator and equilibrated for 15 minutes at room temperature. 40 IA of CellTiter Glo reagent (Promega) was added to measure the relative level of metabolically active cells by quantifying intracellular ATP concentrations. Plates were incubated for 30 minutes at room temperature, and luminescence was measured. Percent viability was normalized to a vehicle control only using the following formula: % viability=100×(Lum_{Sample}−Lum_{LC}) (Lum_{HC}−Lum_{LC}). IC₅₀ values were calculated using XLFit software or Prism (GraphPad Software), as shown in Tables B3-B4, below. Graphical curves were fitted using a nonlinear regression model with a sigmoidal dose response.

TABLE B3

| | Compound (IC₅₀, nM) | | | |
|---|---|---|---|---|
| Cell Line | Tucatinib | Neratinib | 1 | 3 |
| BT-474 | 41.8 | 2.2 | 3.89 | 13.5 |
| MDA MB 175VII | 89 | 7.1 | 9.0 | 43.3 |
| A431 | >10,000 | 43.3 | 1729 | 4441 |
| Ba/F3 ERBB2 wt | 26.5 | 1.1 | 1.5 | 18 |
| Ba/F3 ERBB2 | 356 | 3.5 | 4.4 | 8.1 |
| A775_G776insYVMA | | | | |
| Ba/F3 EGFR wt | >10,000 | 287 | 2753 | |
| Ba/F3 ERBB2-G776VC | 193 | 3.9 | 2.6 | 30 |
| Ba/F3 ERBB2- | 25.3 | 3.4 | 0.65 | 7.1 |
| P780_Y781insGSP | | | | |
| Ba/F3 ERBB2- | 12.5 | 0.79 | <1 | * |
| V777_G778insGC | | | | |
| Ba/F3 ERBB2-V777L | 11 | 0.73 | <1 | * |
| Ba/F3 ERBB2-T798M | 1367 | 20 | 15 | * |
| Ba/F3 ERBB2-L755S | 228 | 6.4 | approx 1 | * |
| Ba/F3 ERBB2-L869R | 63.5 | 2.3 | <1 | * |
| BaF3-EGFR- | * | 62 | 861 | * |
| V769 D770insASV | | | | |
| BaF3 EGFR-D770- | * | 108 | 775 | * |
| N771insSVD | | | | |
| BaF3 EGFR- | * | 481 | >1000 | * |
| H773 V774insNPH | | | | |

*not evaluated

TABLE B4

| | Compound (IC₅₀, nM) | | | | | | |
|---|---|---|---|---|---|---|---|
| Cell Line | 5 | 8 | 9 | 10 | 14 | 150 | 168 |
| BT-474 | 5.72 | 16.7 | 12.3 | 35.5 | 9.60 | 1.05 | 2.17 |
| MDA MB 175VII | * | 562 | 142 | 232 | 30.4 | * | * |
| A431 | 591 | >10000 | 454 | 1083 | 3553 | * | * |
| Ba/F3 ERBB2 wt | 2.3 | 31 | 5.7 | 13.1 | 5.1 | 2.12 | * |
| Ba/F3 ERBB2 | 8.3 | 276 | 20 | 49 | 36 | 8.42 | * |
| A775_G776insYVMA | | | | | | | |
| Ba/F3 ERBB2-G776VC | 3.8 | 117 | 21 | 81 | 13 | 13.6 | * |
| Ba/F3 ERBB2- | 1.1 | 20.4 | 5.5 | 16 | 3.8 | 2.63 | * |
| P780_Y781insGSP | | | | | | | |
| Ba/F3 ERBB2- | 1.3 | 34 | 2.4 | 9.3 | 4 | 3.01 | * |
| V777_G778insGC | | | | | | | |
| Ba/F3 ERBB2-V777L | 1.1 | 11 | 4.1 | 7.3 | 1.5 | 1.73 | * |
| Ba/F3 ERBB2-T798M | 44 | 1496 | 239 | 220 | 482 | 56.9 | * |
| Ba/F3 ERBB2-L755S | 6 | 159 | 35 | 81 | 25 | 11.4 | * |
| Ba/F3 ERBB2-L869R | 2.3 | 52 | 15 | 15 | 9.5 | 4.31 | * |

*not evaluated

TABLE B5

| | Cell Line: Ba/F3 ERBB2 |
|---|---|
| | A775 G776ins YVMA |
| Compound | IC₅₀ (nM) |
| 185 | 5.11 |
| 187 | 19.6 |
| 189 | 2.47 |
| 190 | 34.5 |
| 191 | 177 |
| 192 | 11 |
| 193 | 9.32 |
| 194 | 32 |
| 195 | 86.8 |
| 196 | 9.96 |
| 197 | 13.1 1 |
| 198 | 11.5 |
| 199 | 2.51 |
| 200 | 2.63 |
| 201 | 346 |
| 202 | 49.4 |
| 203 | 5.11 |
| 208 | >500 |
| 209 | >500 |
| 210 | 10.8 |
| 211 | 3.2 |
| 212 | 9.41 |
| 213 | 9.65 |

### Example B3. Detection of phosphorylated ERBB2 (pERBB2) and phosphorylated EGFR (pEGFR)

BT-474 cells were seeded into a 96-well at 2.0*10⁴ cells/100 μl/well.

Compounds were dissolved and serially diluted in DMSO. The compounds were then were added, mixed, and incubated for four hours at 37° C., 5% CO₂. Compounds were added using four-fold dilutions at final concentrations ranging from 10 μM to 0.01 nM.

Following the four hour incubation with compounds, cell lysates were prepared. Plates were centrifuged for 5 min at 3000 RPM, and supernatant was removed from each well. Cells were washed 3 times by resuspension in 150 μl PBS, followed by centrifugation and removal of the supernatant, as above. 100 μl of cell lysis buffer (Boston BioProducts, cat #BP-115D) supplied with 1× complete ULTRA cocktail inhibitor (Thermo Scientific™, cat #78443) was then added to the washed cells. Cells were incubated with lysis buffer for 1 hour at 4° C., and then stored at −80° C.

Enzyme-linked immunosorbent assays (ELISA) were performed to measure phosphorylated ERBB2 levels. A capture antibody able to detect phosphorylated and non-phosphorylated ERBB2 (R&D Systems, cat #841425) was added to ELISA plates and incubated at 4° C. overnight. The next day, plates were washed with PBS+0.05% Tween20 (PBST). 150 μl of 5% BSA blocking solution was added for 1 hour at room temperature, with shaking. Plates were washed with PBST. Cell lysates were thawed and 100 μl of lysate was added to the ELISA plate. The plates were incubated for 2 hours at room temperature, with shaking. ELISA plates were then washed with PBST and 100 μl of an HRP-labeled detection antibody that binds phosphorylated tyrosine (R&D Systems, cat #841913) was added to each well. Plates were incubated for 1 hour at room temperature, with shaking. Plates were then washed with PBST, and 100 μl TMB substrate solution (R&D Systems, cat #DY999) was added. Plate were incubated in the dark for 20 minutes at room temperature. 50 μl of Stop solution (R&D Systems, cat #DY994) (50 μl) was added to each well and mixed.

Optical density at 450 nm was read on an EnSpire plate reader (Perkin Elmer). The remaining kinase activity by calculated using the following formula: % Relative activity=100×(A450ₛₐₘₚₗₑ−A450_{LC})/(A450_{HC}−A450_{LC}). The low and high control values (“LC” and “HC”) were generated from lysate from wells without cells or with cells treated with 0.1% DMSO, respectively. IC₅₀ values were calculated using XLFit software using a nonlinear regression model with a sigmoidal dose response, as shown in Table B6 below.

TABLE B6

| Compound | PERBB2 IC₅₀ (nM) |
|---|---|
| Tucatinib | 23.1 |
| Neratinib | 35.6 |
| 1 | 10.3 |
| 3 | 152 |
| 8 | 72.9 |
| 9 | 43.9 |
| 10 | 74.9 |
| 14 | 14.3 |
| 150 | 7.05 |
| 168 | 17 |
| 170 | 14 |
| 179 | 121 |
| 180 | 23.4 |
| 181 | 22.8 |
| 182 | 13.2 |
| 185 | 17.3 |
| 186 | 16.1 |
| 187 | 30.5 |
| 188 | 78 |
| 189 | 1.87 |
| 190 | 24.9 |
| 191 | 93.2 |
| 192 | 9.51 |
| 193 | 13.8 |
| 194 | 18.2 |
| 195 | 104 |
| 196 | 8.36 |
| 197 | 11.4 |
| 198 | 6.23 |
| 199 | 4.7 |
| 200 | 3.7 |
| 201 | 153 |
| 202 | 21.8 |
| 203 | 6.05 |
| 208 | 1180 |
| 209 | 1250 |
| 210 | 12.1 |
| 211 | 36.6 |
| 212 | 52.3 |
| 213 | 32.4 |

Enzyme-linked immunosorbent assays (ELISA) were performed to measure phosphorylated EGFR levels using A431 cells (10% FBS). A431 (1.0*10⁴ cells/40 μl/well) cells were seeded in 384 well. Compounds were dissolved in DMSO, serially diluted in DMSO and then were added, mixed, and incubated for 4 hours at 37° C., 5% CO₂. Following the 4-hours incubation, cells were stimulated for 10 minutes with EGF (Invitrogen, cat #PHG0311) at a final concentration of 30 ng/mL in the incubator. The media was aspirated and cells were lysed in 10 μL lysis buffer with protease and phosphatase inhibitors (PerkinElmer, cat #ALSU-PEGFR-A50K). The plates were placed on a shaker for 5 minutes and then incubated for 30 min at 4° C. for complete lysis. The lysate was transferred to an Optiplate (Perkin Elmer, cat #6007290).

Acceptor mix (PerkinElmer, cat #ALSU-PEGFR-A50K) was prepared just before use and 5 μL was dispensed to all the wells, followed by a 1.5-2 h incubation at room temperature in dark. The donor mix (PerkinElmer, cat #ALSU-PEGFR-A50K) was prepared under low light conditions prior to use and 50 of donor mix was added to all the wells under subdued lighting or green filters. The plates were placed on a shaker for 5 min, sealed, and incubated overnight at room temperature in dark. Plates were read on the Envision (PerkinElmer) using standard AlphaLISA settings.

The percentage of inhibition on EGFR phosphorylation was calculated following equation: % Inhibition=100×(Lum_{HC}−Lum_{Sample})/(Lum_{HC}−Lum_{LC}). The low and high controls (LC/HC) are generated from lysate from wells with cells treated with DMSO or 10 mM Staurosporine (BioAustralis, cat #BIA-S1086), respectively. IC50 values were calculated by fitting the Curve using XLfit (v5.3.1.3), equation 201: Y=Bottom+(Top−Bottom)/(1+10\hat{ }((LogIC50−X)*HillSlope)). The IC₅₀ values are shown in Table B7 below.

TABLE B7

| Compound | pEGFR IC₅₀ (nM) |
|---|---|
| 150 | 233 |
| 168 | 70.9 |
| 170 | 418 |
| 179 | 2550 |
| 180 | 1590 |
| 181 | 971 |
| 182 | 1490 |
| 185 | 680 |
| 186 | 74.5 |
| 187 | 434 |
| 188 | 297 |
| 189 | 106 |
| 190 | 1220 |
| 191 | >10.0E+03 |
| 192 | 1350 |
| 193 | 658 |
| 194 | 804 |
| 195 | 7070 |
| 196 | 943 |
| 197 | 555 |
| 198 | 508 |
| 199 | 57.5 |
| 200 | 69.6 |
| 201 | 1970 |
| 202 | 543 |
| 203 | 45.5 |
| 208 | 2260 |
| 209 | >10.0E+03 |
| 210 | 1850 |
| 211 | 101 |
| 212 | 377 |
| 213 | 579 |

1. A compound of formula (I) 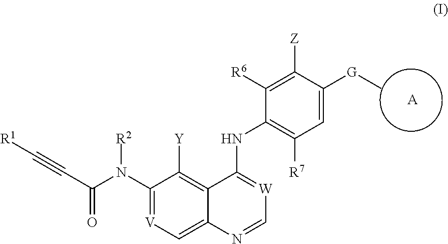
or a pharmaceutically acceptable salt thereof, wherein:
ring A is 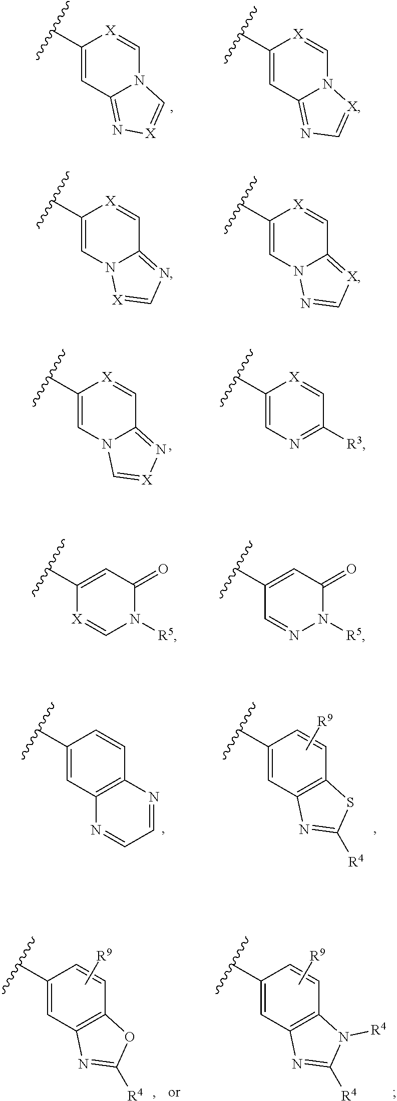
V is N or C—R⁸;
W is N or C—CN;
each X is independently N or CH;
G is CH₂, CH(CH₃), 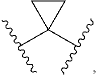 [SMILES: *C1(*)CC1]
O, or S;
Y is H, F, or —O(C₁-C₃ alkyl);
Z is H, F, Cl, C₁-C₂ alkyl, or cyclopropyl;
R¹ is H, C₁-C₄ alkyl, C₃-C₆ cycloalkyl, or 4- to 6-membered heterocyclyl containing 1-2 ring heteroatoms selected from the group consisting of N, O and S, wherein the C₁-C₃ alkyl is optionally substituted by 1-3 substituents selected from the group consisting of F, —OH, —OPO₃²—, —N(C₁-C₃ alkyl)(C₁-C₃ alkyl), and 4- to 6-membered heterocyclyl containing 1-2 ring heteroatoms selected from the group consisting of N and O; and wherein the C₃-C₆ cycloalkyl and 4- to 6-membered heterocyclyl are optionally substituted by 1-3 substituents selected from the group consisting of F, —OH, C₁-C₃ alkyl, —N(C₁-C₃ alkyl)(C₁-C₃ alkyl), and 4- to 6-membered heterocyclyl containing 1-2 ring heteroatoms selected from the group consisting of N and O;
R² is H, C₁-C₃ alkyl or cyclopropyl;
R³ is H, —CD₃, C₁-C₃ alkyl, —CF₂H, —CF₃, —CFH—CFH₂, allyl, —CH₂-cyclopropyl, cyclopropyl, —CN, —OR⁴, —SR⁴, —S(O)(C₁-C₃ alkyl), or —S(O)₂(C₁-C₃ alkyl);
each R⁴ is independently H, C₁-C₃ alkyl, —CF₂H, —CF₃, or cyclopropyl;
R⁵ is C₁-C₃ alkyl, —CD₃, —CF₂H, —CF₃, allyl, —CH₂-cyclopropyl, cyclopropyl, or —OR⁴;
R⁶ is H or F;
R⁷ is H or F;
R⁸ is H or —O(C₁-C₃ alkyl), wherein the C₁-C₃ alkyl is optionally substituted by 1-4 substituents selected from the group consisting of —F, —OH, —OR^{8a}, and —NR^{8a}R^{8b}, wherein each R^{8a} and R^{8b} are independently H or C₁-C₃ alkyl, or wherein each pair of geminal R^{8a} and R^{8b} may be taken together with the nitrogen atom to which they are attached to form an N-heterocycloalkyl wherein the N-heterocycloalkyl is optionally substituted by C₁-C₃ alkyl; and
R⁹ is H, halogen, C₁-C₃ alkyl, —CF₂H, —CF₃, cyclopropyl, —CN or —OR⁴.

2. The compound of claim 1, or a pharmaceutically acceptable salt thereof, wherein V is C—R⁸, and
R⁸ is —O(C₂-C₃ alkyl), wherein the C₂-C₃ alkyl is substituted by 1-2 substituents selected from the group consisting of —OH and —NR^{8a}R^{8b}, wherein each R^{8a} and R^{8b} are independently H or C₁-C₃ alkyl.

3-5. (canceled)

6. The compound of claim 1, or a pharmaceutically acceptable salt thereof, wherein W is N.

7. (canceled)

8. The compound of claim 1, wherein the compound of formula (I) is a compound of formula (II) 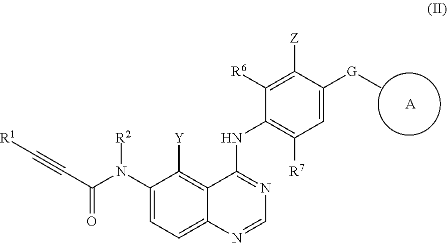
or a pharmaceutically acceptable salt thereof, wherein:
ring A is 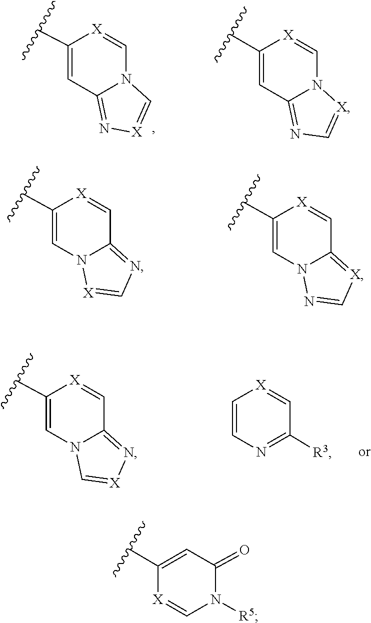
each X is independently N or CH;
G is CH₂, O, or S;
Y is H, F, or —O(C₁-C₃ alkyl);
Z is H, F, Cl, C₁-C₂ alkyl, or cyclopropyl;
R¹ is H, C₁-C₃ alkyl, C₃-C₆ cycloalkyl, or 4- to 6-membered heterocyclyl containing 1-2 ring heteroatoms selected from the group consisting of N, O and S, wherein the C₁-C₃ alkyl, C₃-C₆ cycloalkyl, and 4- to 6-membered heterocyclyl are optionally substituted by 1-3 substituents selected from the group consisting of F, —OH, —N(C₁-C₃ alkyl)(C₁-C₃ alkyl), and 4- to 6-membered heterocyclyl containing 1-2 ring heteroatoms selected from the group consisting of N and O;
R² is H, C₁-C₃ alkyl or cyclopropyl;
R³ is H, —CD₃, C₁-C₃ alkyl, —CF₂H, —CF₃, allyl, —CH₂-cyclopropyl, cyclopropyl, —CN, —OR⁴, —SR⁴, —S(O)(C₁-C₃ alkyl), or —S(O)₂(C₁-C₃ alkyl);
R⁴ is H, C₁-C₃ alkyl, —CF₂H, —CF₃, or cyclopropyl;
R⁵ is C₁-C₃ alkyl, —CD₃, —CF₂H, —CF₃, allyl, —CH₂-cyclopropyl, cyclopropyl, or —OR⁴;
R⁶ is H or F; and
R⁷ is H or F.

9. The compound of claim 1, or a pharmaceutically acceptable salt thereof, wherein:
ring A is 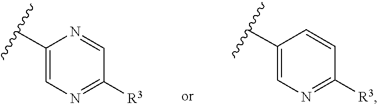
and
R³ is H, —CD₃, C₁-C₂ alkyl, —CFH—CFH₂, CF₂H, CF₃, cyclopropyl, —CN, —OR⁴, —SR⁴, —S(O)(C₁-C₂ alkyl), or —S(O)₂(C₁-C₂ alkyl).

10-14. (canceled)

15. The compound of claim 1, or a pharmaceutically acceptable salt thereof, wherein:
ring A is 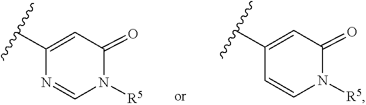
and R⁵ is C₁-C₂ alkyl, —CD₃, CF₂H, CF₃, allyl, —CH₂-cyclopropyl, cyclopropyl, or —OR⁴.

16-20. (canceled)

21. The compound of claim 1, or a pharmaceutically acceptable salt thereof, wherein:
ring A is

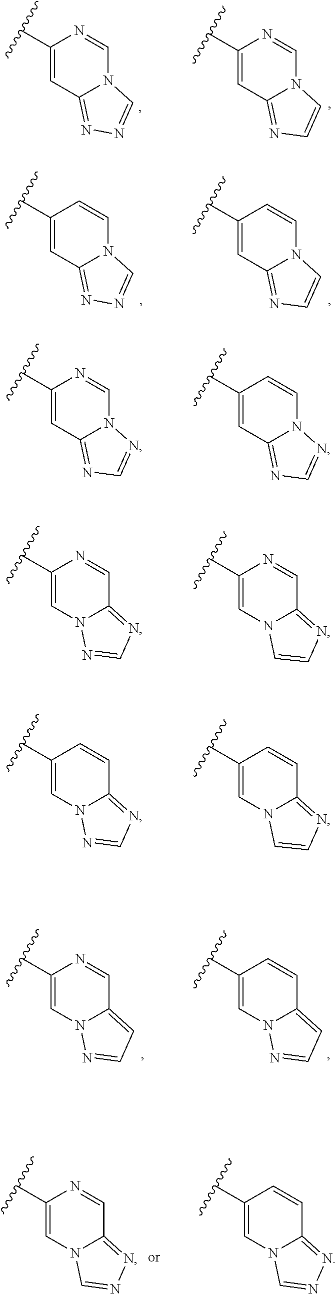

22-23. (canceled)

24. The compound of claim 1, or a pharmaceutically acceptable salt thereof, wherein:
Z is H, C₁, or —CH₃.

25. (canceled)

26. The compound of claim 1, or a pharmaceutically acceptable salt thereof, wherein:
R¹ is H, C₁-C₂ alkyl, cyclopropyl, or 4- to 6-membered heterocyclyl containing 1-2 ring heteroatoms selected from the group consisting of N, O and S, wherein the C₁-C₂ alkyl, cyclopropyl, and 4- to 6-membered heterocyclyl are optionally substituted by 1-3 substituents selected from the group consisting of F, —OH, —N(C₁-C₃ alkyl)(C₁-C₃ alkyl), and 4- to 6-membered heterocyclyl containing 1-2 ring heteroatoms selected from the group consisting of N and O.

27-29. (canceled)

30. The compound of claim 1, or a pharmaceutically acceptable salt thereof, wherein:
Y is H, F, or —O(C₁-C₂ alkyl).

31-32. (canceled)

33. The compound of claim 1, or a pharmaceutically acceptable salt thereof, wherein:
R² is H or C₁-C₃ alkyl.

34-35. (canceled)

36. The compound of claim 1, or a pharmaceutically acceptable salt thereof, wherein:
G is O.

37-44. (canceled)

45. A compound selected from the group consisting of: 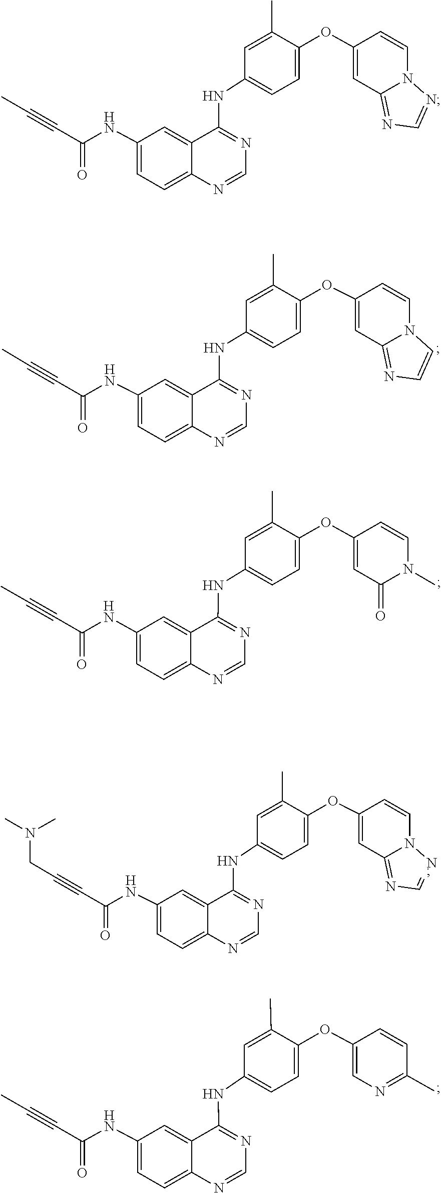 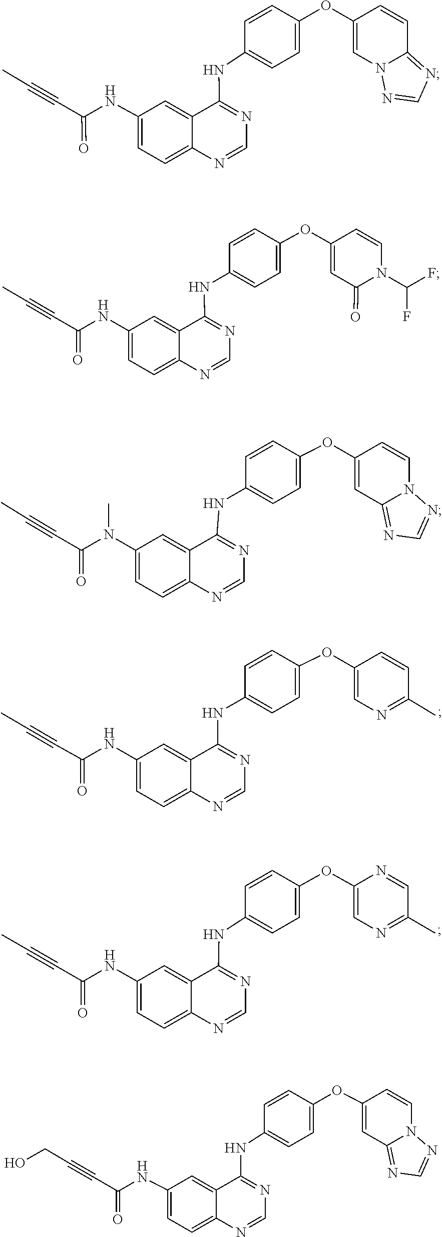 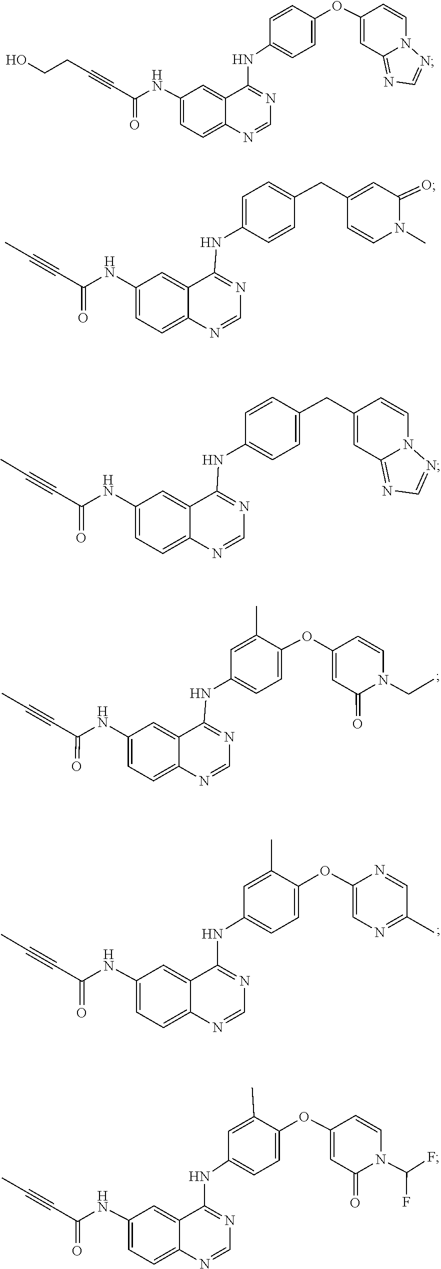 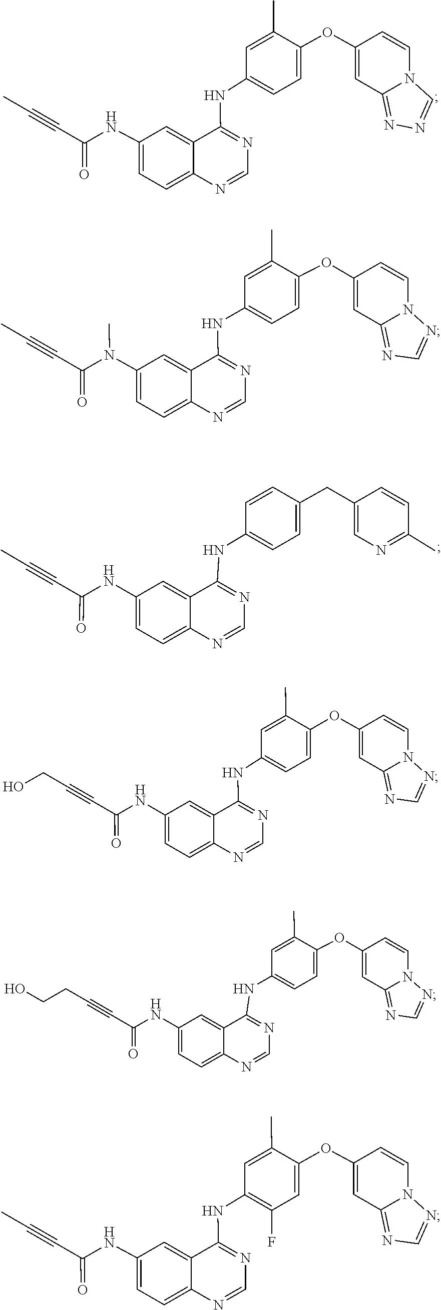 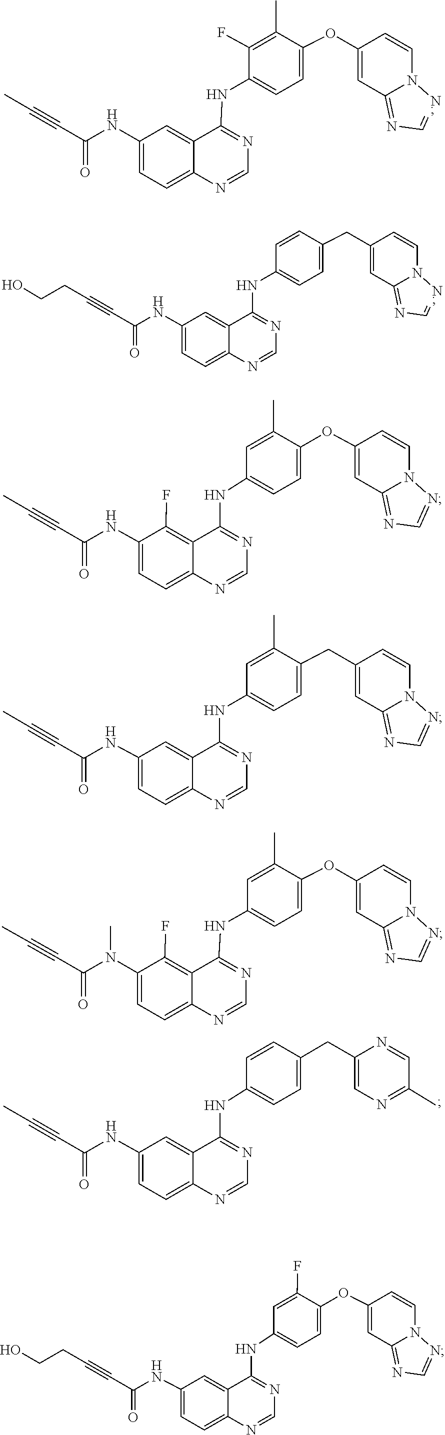 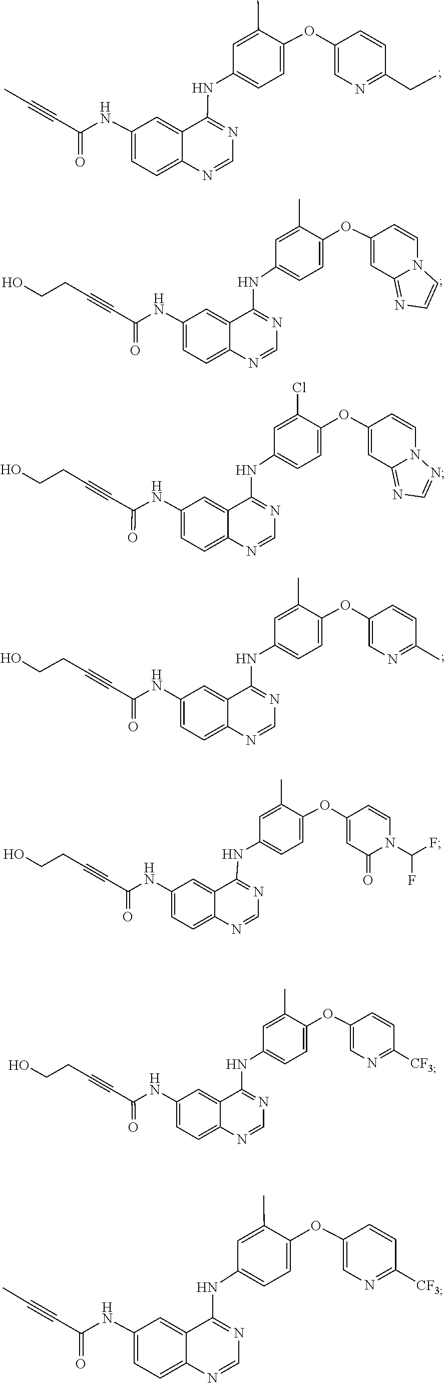 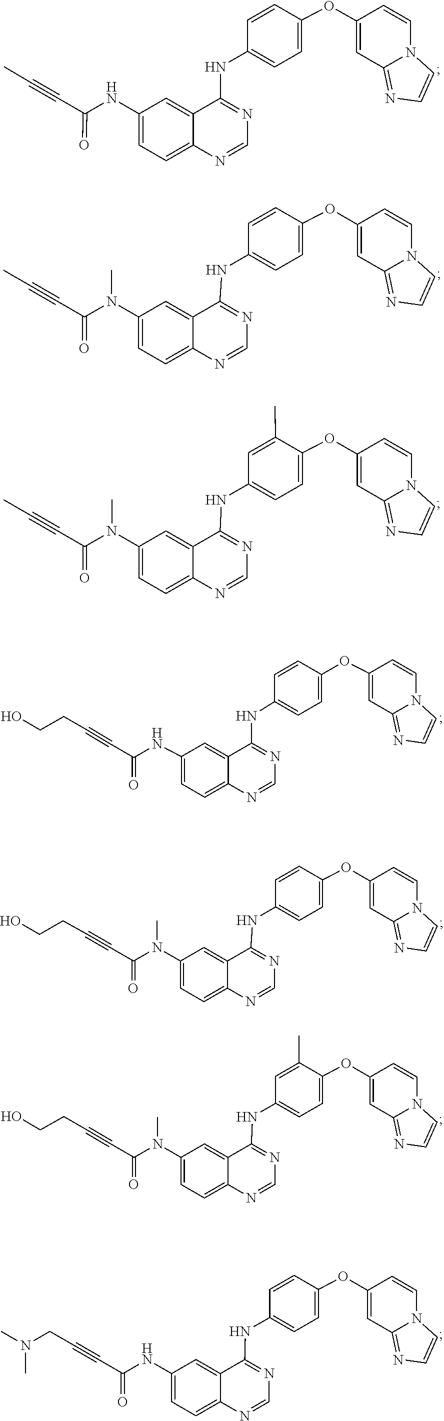 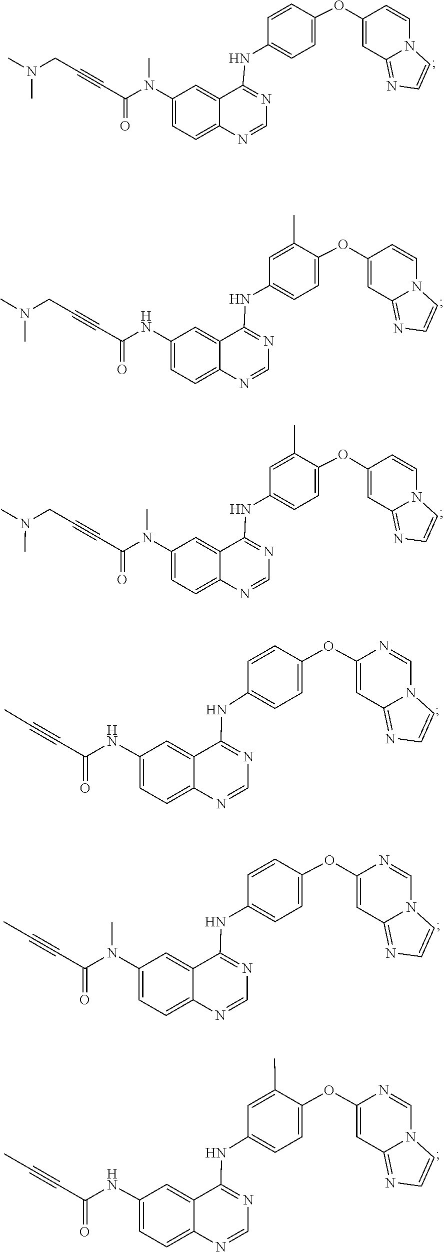 [SMILES: CN(C)CC#CC(=O)N(C)c1ccc2ncnc(Nc3ccc(Oc4ccn5ccnc5c4)cc3)c2c1 ; Cc1cc(Nc2ncnc3ccc(NC(=O)C#CCN(C)C)cc23)ccc1Oc1ccn2ccnc2c1 ; Cc1cc(Nc2ncnc3ccc(N(C)C(=O)C#CCN(C)C)cc23)ccc1Oc1ccn2ccnc2c1 ; CC#CC(=O)Nc1ccc2ncnc(Nc3ccc(Oc4cc5nccn5cn4)cc3)c2c1 ; CC#CC(=O)N(C)c1ccc2ncnc(Nc3ccc(Oc4cc5nccn5cn4)cc3)c2c1 ; CC#CC(=O)Nc1ccc2ncnc(Nc3ccc(Oc4cc5nccn5cn4)c(C)c3)c2c1] 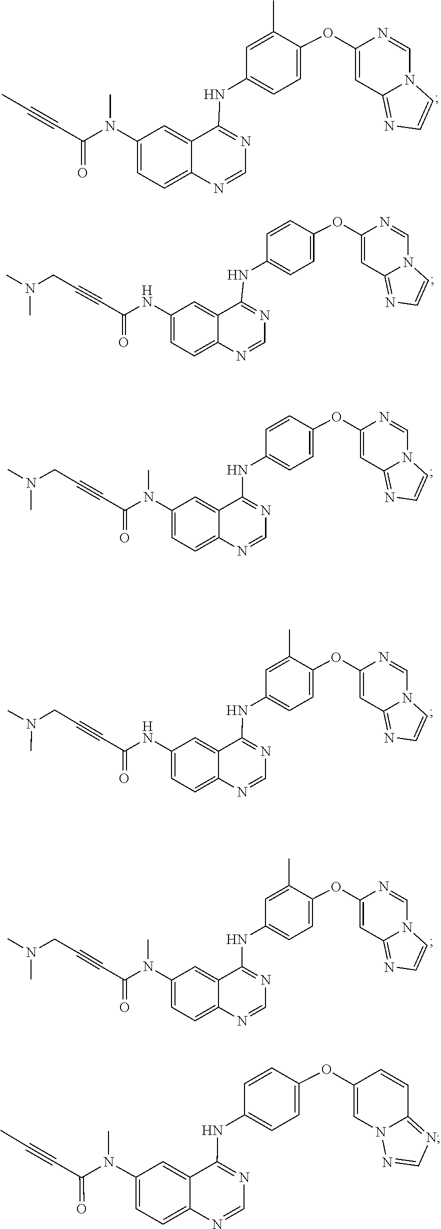 [SMILES: CC#CC(=O)N(C)c1ccc2ncnc(Nc3ccc(Oc4cc5nccn5cn4)c(C)c3)c2c1 ; CN(C)CC#CC(=O)Nc1ccc2ncnc(Nc3ccc(Oc4cc5nccn5cn4)cc3)c2c1 ; CN(C)CC#CC(=O)N(C)c1ccc2ncnc(Nc3ccc(Oc4cc5nccn5cn4)cc3)c2c1 ; Cc1cc(Nc2ncnc3ccc(NC(=O)C#CCN(C)C)cc23)ccc1Oc1cc2nccn2cn1 ; Cc1cc(Nc2ncnc3ccc(N(C)C(=O)C#CCN(C)C)cc23)ccc1Oc1cc2nccn2cn1 ; CC#CC(=O)N(C)c1ccc2ncnc(Nc3ccc(Oc4ccc5ncnn5c4)cc3)c2c1] 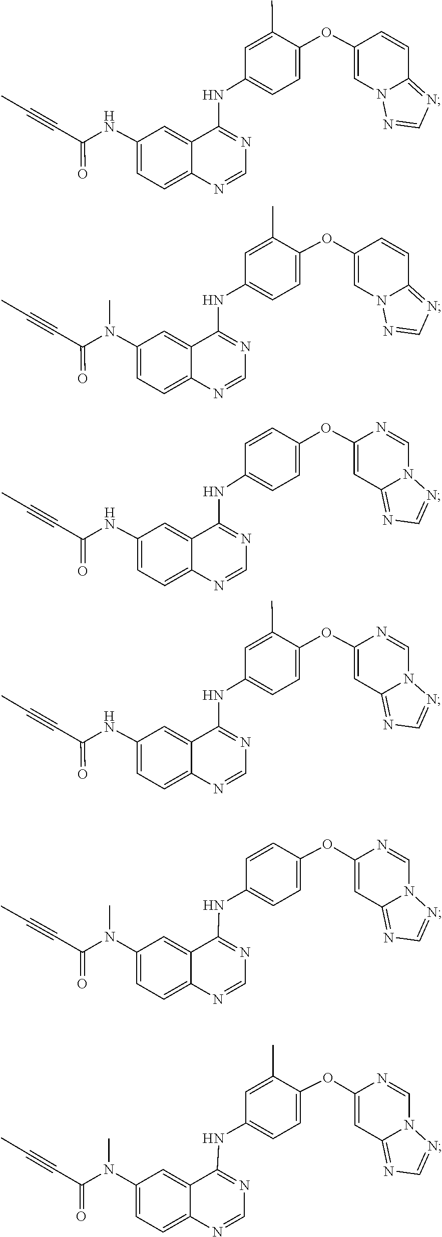 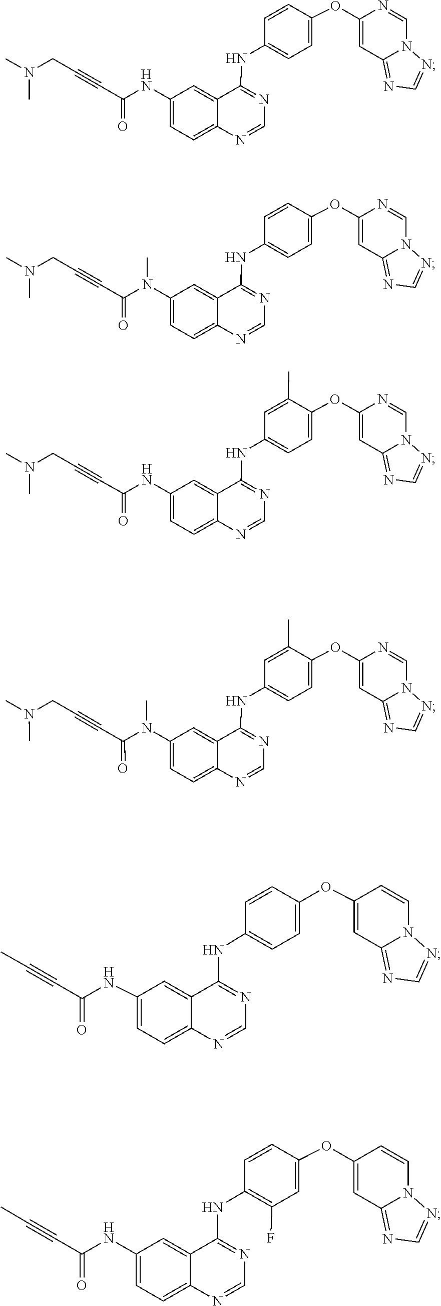 [SMILES: CN(C)CC#CC(=O)Nc1ccc2ncnc(Nc3ccc(Oc4cc5ncnn5cn4)cc3)c2c1 ; CN(C)CC#CC(=O)N(C)c1ccc2ncnc(Nc3ccc(Oc4cc5ncnn5cn4)cc3)c2c1 ; Cc1cc(Nc2ncnc3ccc(NC(=O)C#CCN(C)C)cc23)ccc1Oc1cc2ncnn2cn1 ; Cc1cc(Nc2ncnc3ccc(N(C)C(=O)C#CCN(C)C)cc23)ccc1Oc1cc2ncnn2cn1 ; CC#CC(=O)Nc1ccc2ncnc(Nc3ccc(Oc4ccn5ncnc5c4)cc3)c2c1 ; CC#CC(=O)Nc1ccc2ncnc(Nc3ccc(Oc4ccn5ncnc5c4)cc3F)c2c1] 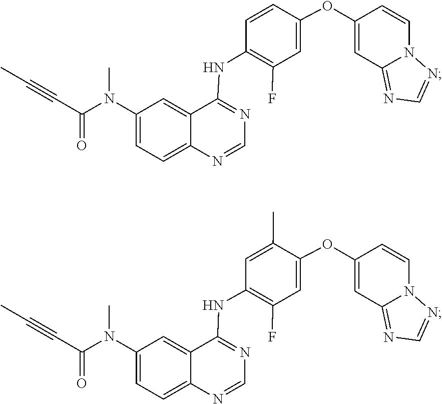 [SMILES: CC#CC(=O)N(C)c1ccc2ncnc(Nc3ccc(Oc4ccn5ncnc5c4)cc3F)c2c1 ; CC#CC(=O)N(C)c1ccc2ncnc(Nc3cc(C)c(Oc4ccn5ncnc5c4)cc3F)c2c1] 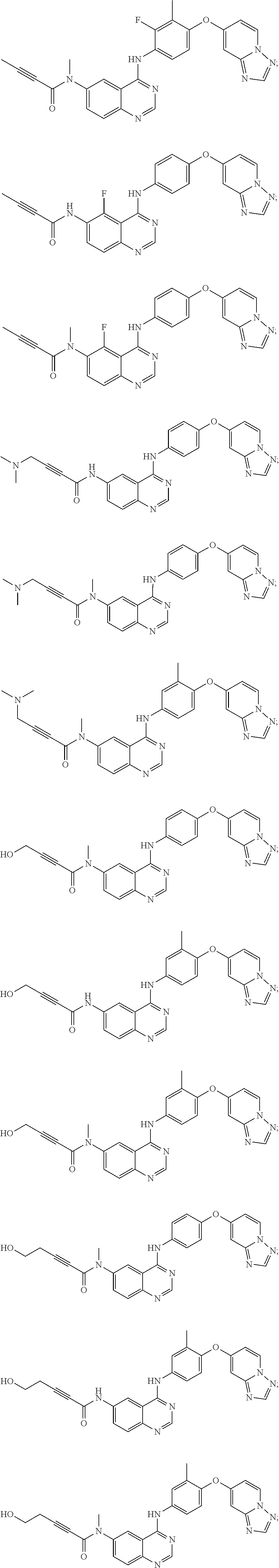 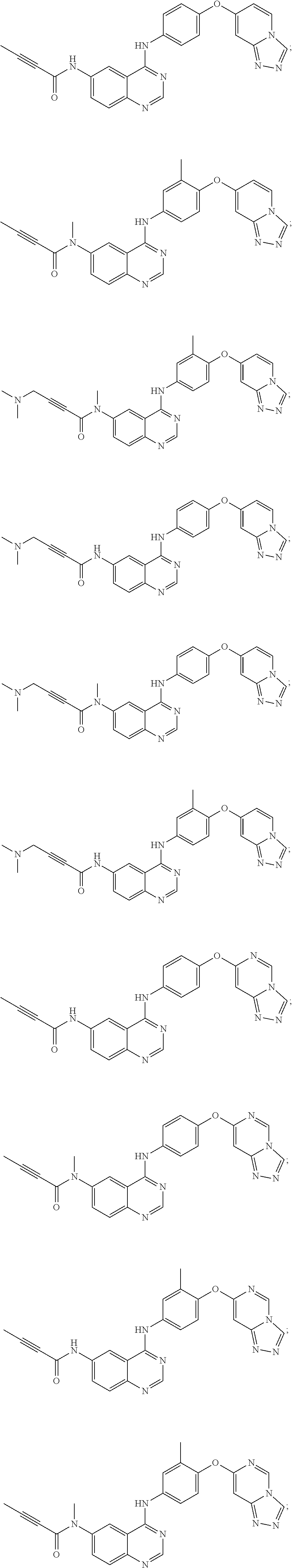 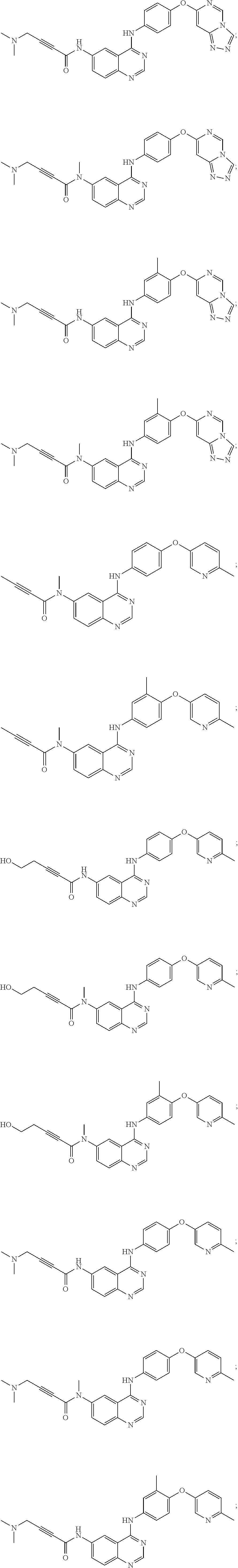 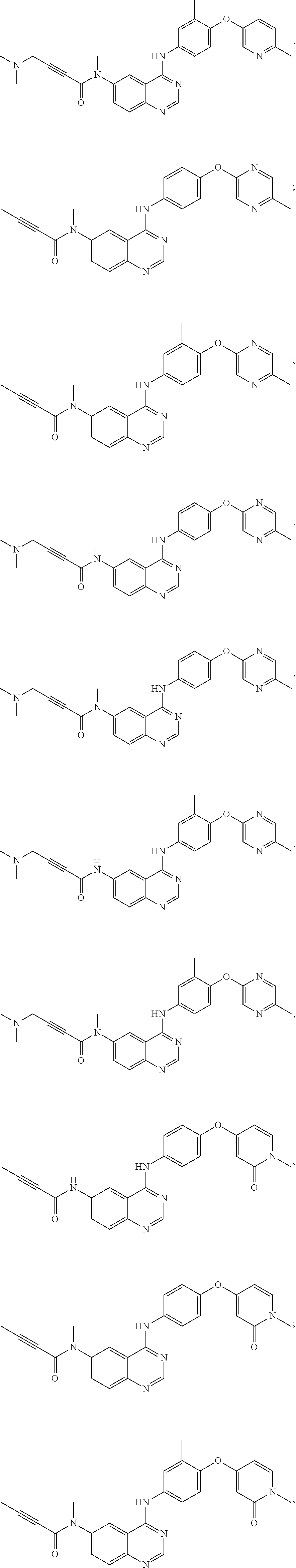 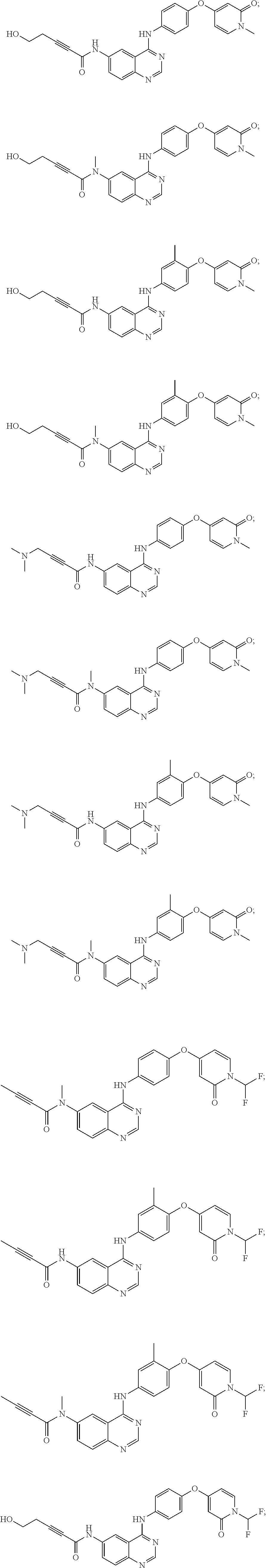 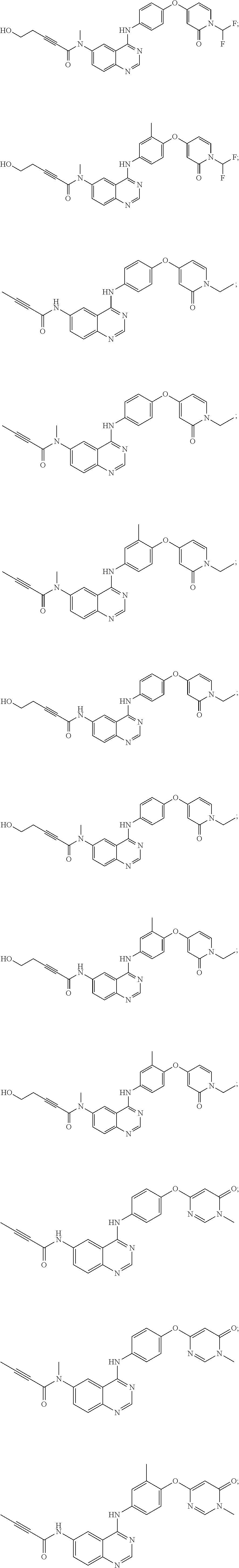 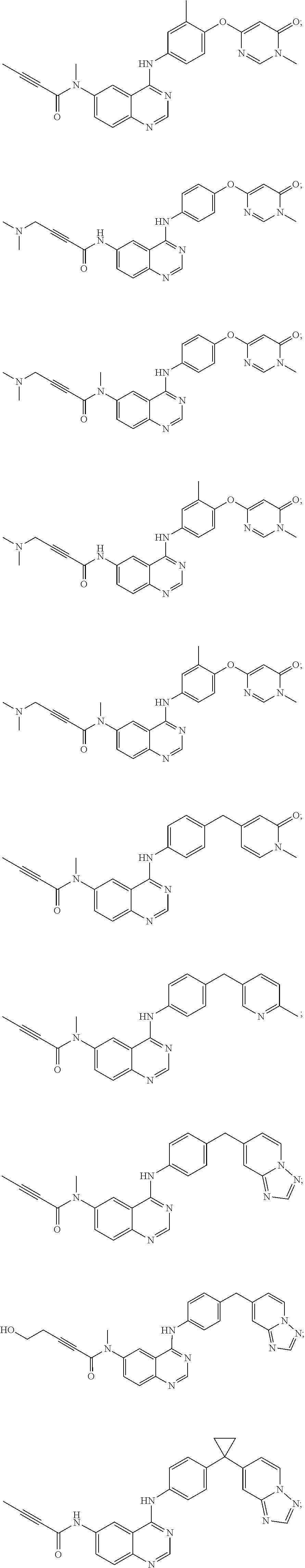 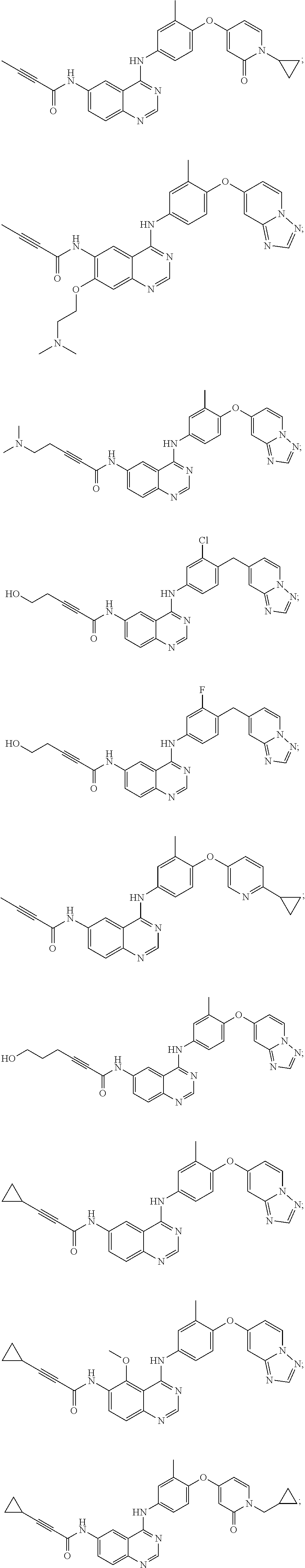 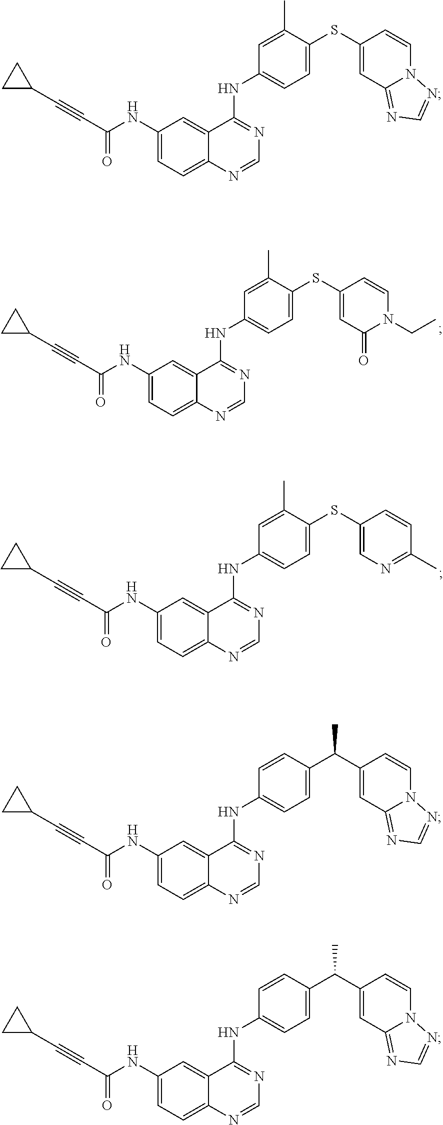 [SMILES: Cc1cc(Nc2ncnc3ccc(NC(=O)C#CC4CC4)cc23)ccc1Sc1ccn2ncnc2c1 ; CCn1ccc(Sc2ccc(Nc3ncnc4ccc(NC(=O)C#CC5CC5)cc34)cc2C)cc1=O ; Cc1ccc(Sc2ccc(Nc3ncnc4ccc(NC(=O)C#CC5CC5)cc34)cc2C)cn1 ; C[C@H](c1ccc(Nc2ncnc3ccc(NC(=O)C#CC4CC4)cc23)cc1)c1ccn2ncnc2c1 ; C[C@@H](c1ccc(Nc2ncnc3ccc(NC(=O)C#CC4CC4)cc23)cc1)c1ccn2ncnc2c1] 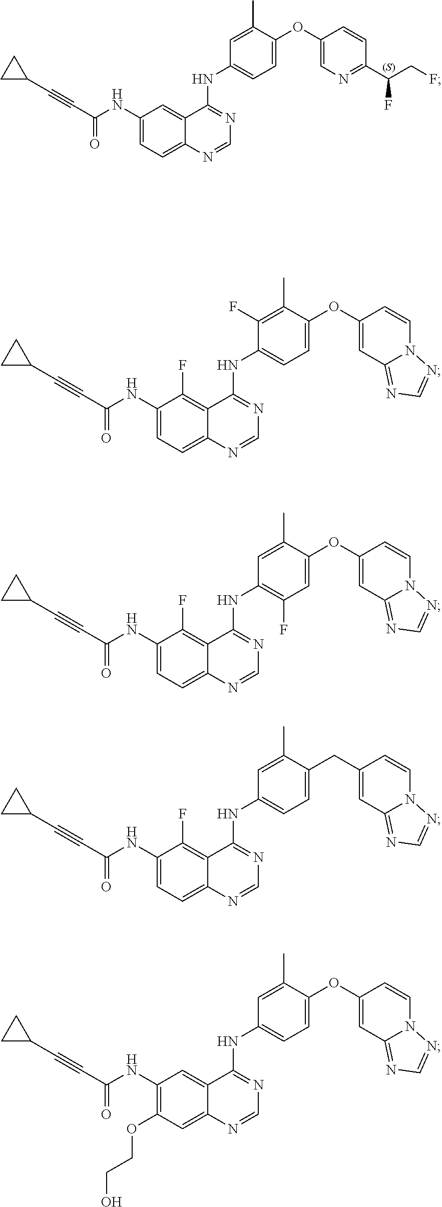 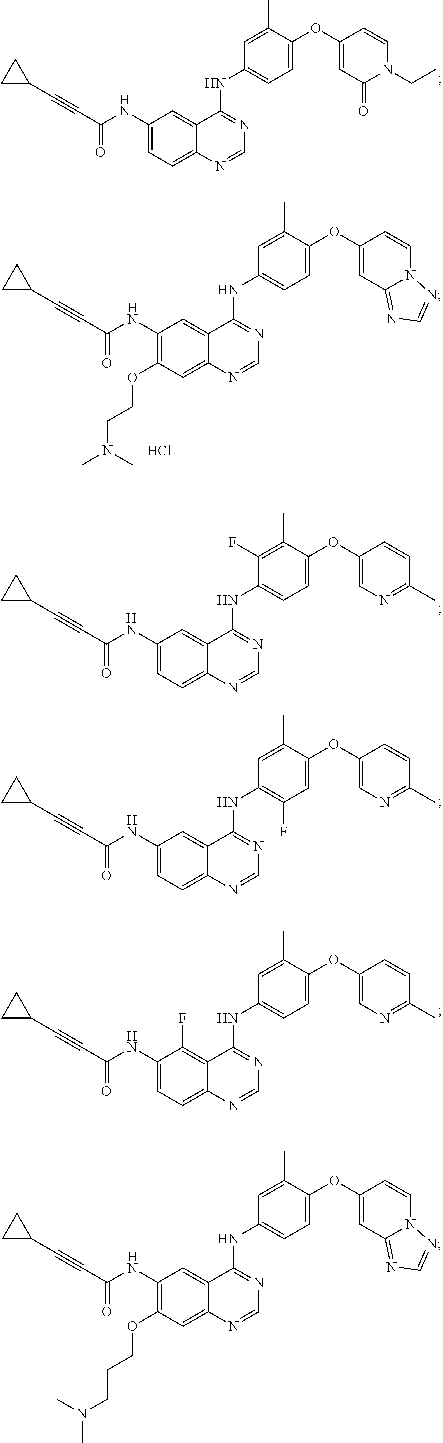 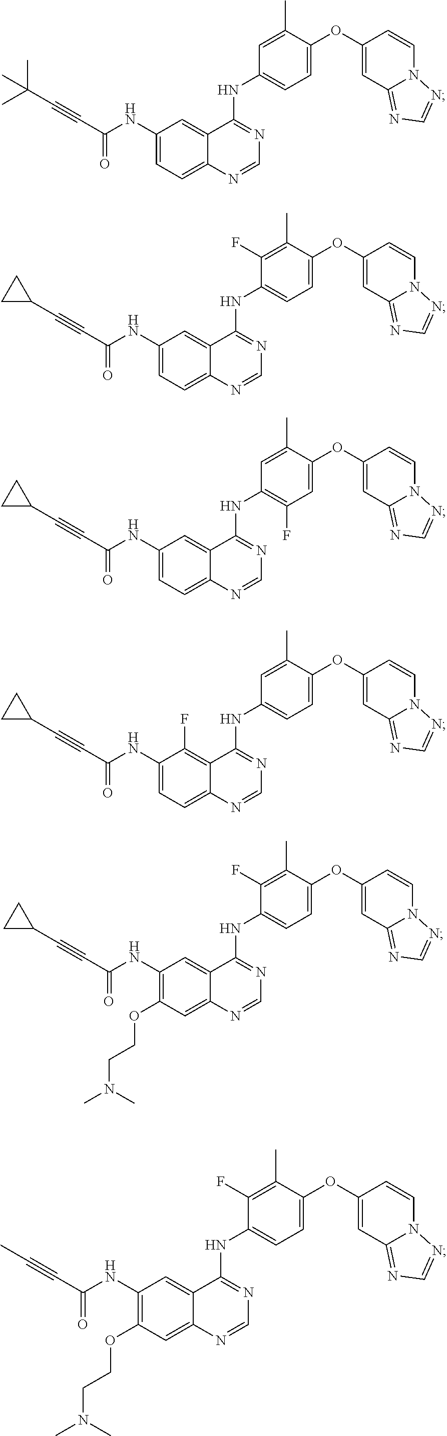 [SMILES: Cc1cc(Nc2ncnc3ccc(NC(=O)C#CC(C)(C)C)cc23)ccc1Oc1ccn2ncnc2c1 ; Cc1c(Oc2ccn3ncnc3c2)ccc(Nc2ncnc3ccc(NC(=O)C#CC4CC4)cc23)c1F ; Cc1cc(Nc2ncnc3ccc(NC(=O)C#CC4CC4)cc23)c(F)cc1Oc1ccn2ncnc2c1 ; Cc1cc(Nc2ncnc3ccc(NC(=O)C#CC4CC4)c(F)c23)ccc1Oc1ccn2ncnc2c1 ; Cc1c(Oc2ccn3ncnc3c2)ccc(Nc2ncnc3cc(OCCN(C)C)c(NC(=O)C#CC4CC4)cc23)c1F ; CC#CC(=O)Nc1cc2c(Nc3ccc(Oc4ccn5ncnc5c4)c(C)c3F)ncnc2cc1OCCN(C)C] 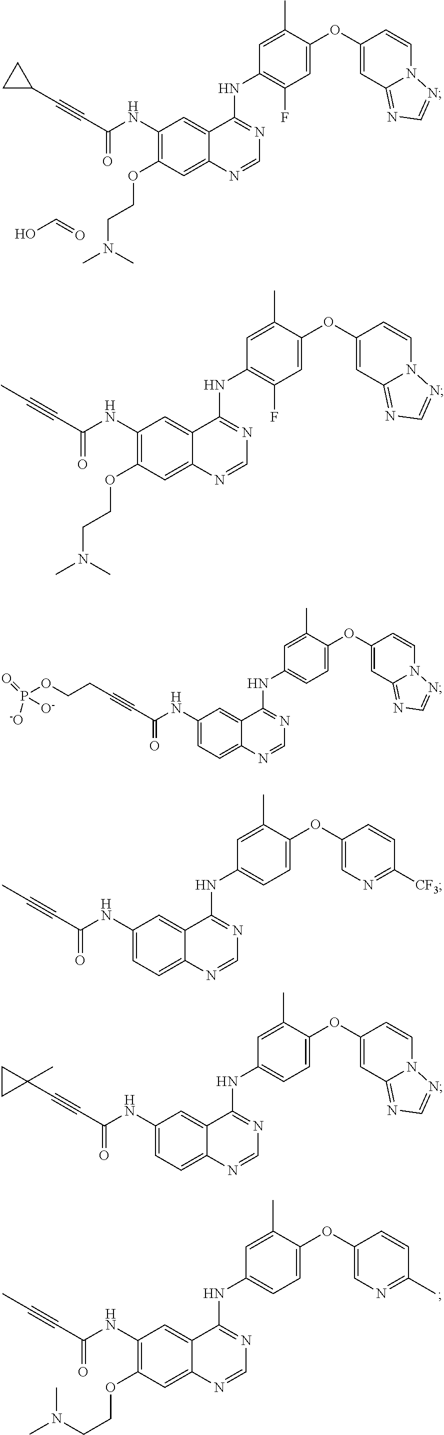 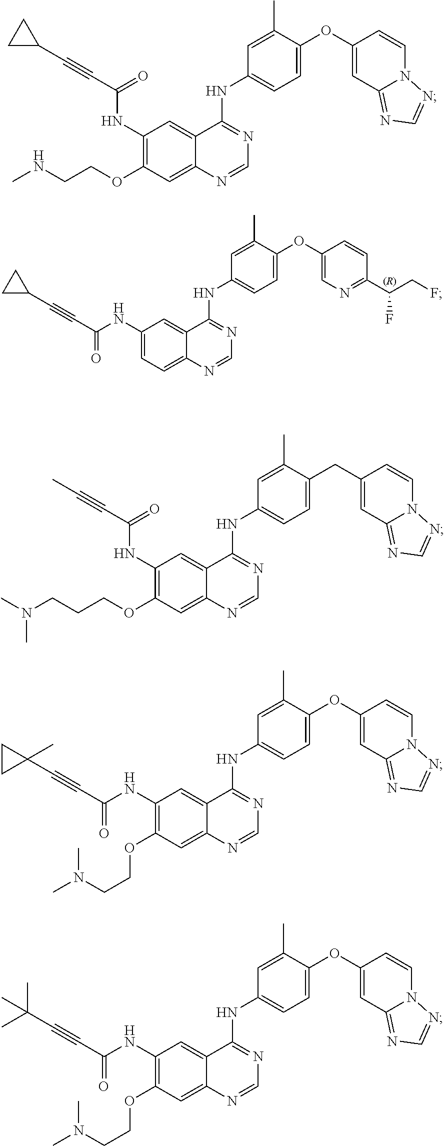 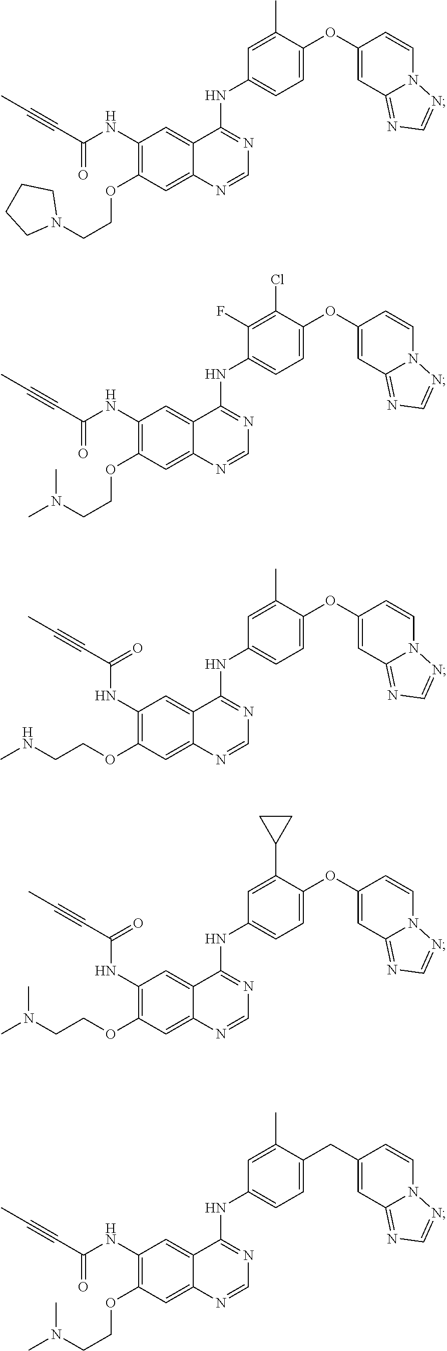 [SMILES: CC#CC(=O)Nc1cc2c(Nc3ccc(Oc4ccn5ncnc5c4)c(C)c3)ncnc2cc1OCCN1CCCC1 ; CC#CC(=O)Nc1cc2c(Nc3ccc(Oc4ccn5ncnc5c4)c(Cl)c3F)ncnc2cc1OCCN(C)C ; CC#CC(=O)Nc1cc2c(Nc3ccc(Oc4ccn5ncnc5c4)c(C)c3)ncnc2cc1OCCNC ; CC#CC(=O)Nc1cc2c(Nc3ccc(Oc4ccn5ncnc5c4)c(C4CC4)c3)ncnc2cc1OCCN(C)C ; CC#CC(=O)Nc1cc2c(Nc3ccc(Cc4ccn5ncnc5c4)c(C)c3)ncnc2cc1OCCN(C)C] 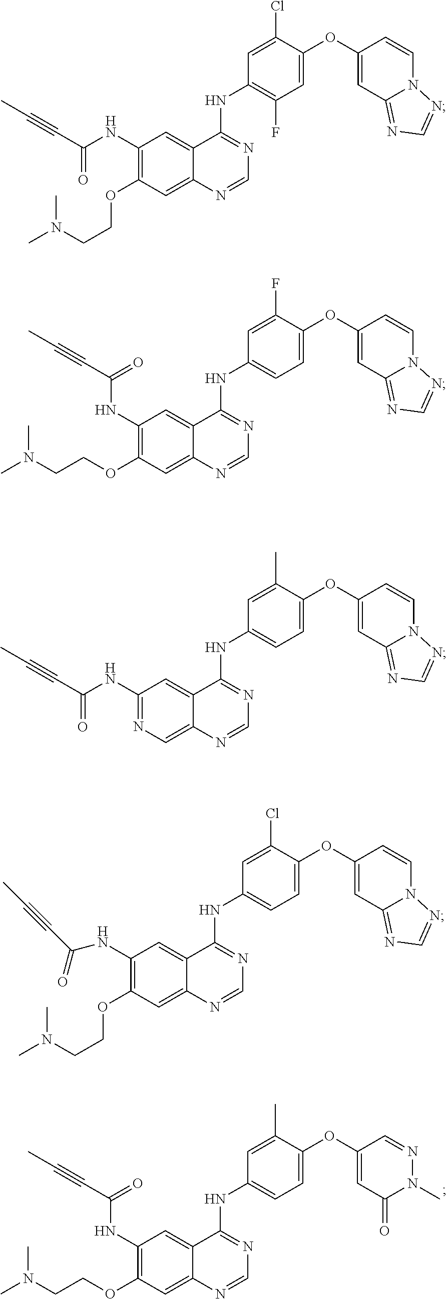 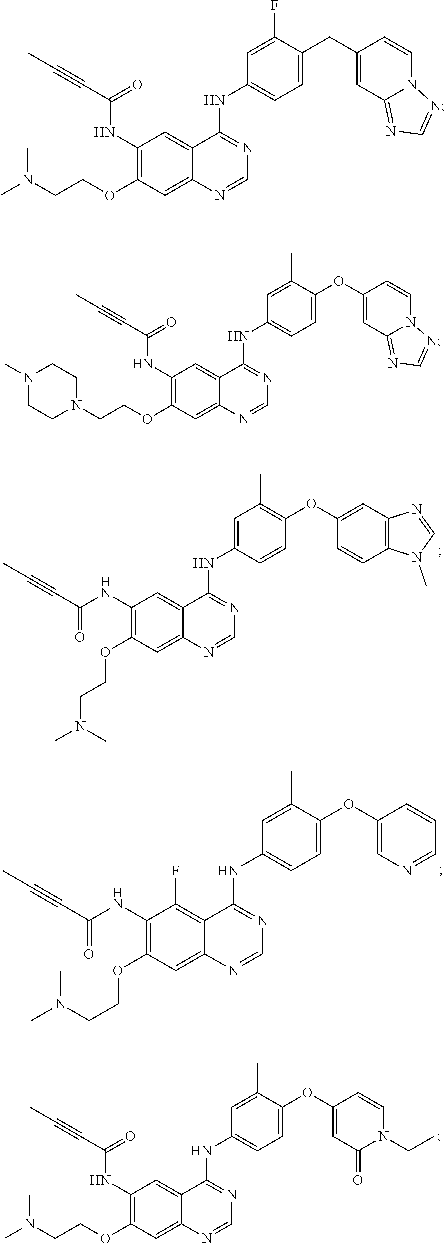 [SMILES: CC#CC(=O)Nc1cc2c(Nc3ccc(Cc4ccn5ncnc5c4)c(F)c3)ncnc2cc1OCCN(C)C ; CC#CC(=O)Nc1cc2c(Nc3ccc(Oc4ccn5ncnc5c4)c(C)c3)ncnc2cc1OCCN1CCN(C)CC1 ; CC#CC(=O)Nc1cc2c(Nc3ccc(Oc4ccc5c(c4)ncn5C)c(C)c3)ncnc2cc1OCCN(C)C ; CC#CC(=O)Nc1c(OCCN(C)C)cc2ncnc(Nc3ccc(Oc4cccnc4)c(C)c3)c2c1F ; CC#CC(=O)Nc1cc2c(Nc3ccc(Oc4ccn(CC)c(=O)c4)c(C)c3)ncnc2cc1OCCN(C)C] 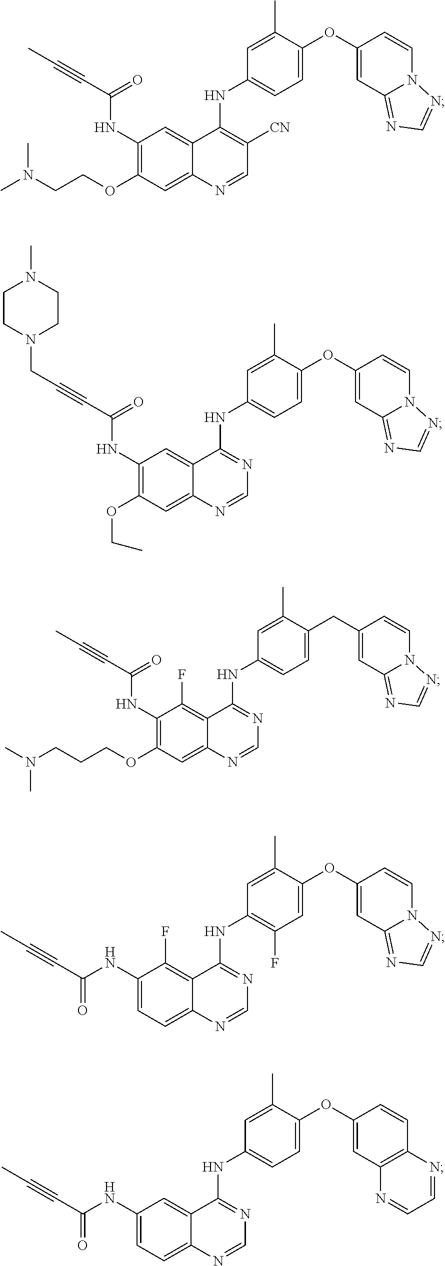 [SMILES: CC#CC(=O)Nc1cc2c(Nc3ccc(Oc4ccn5ncnc5c4)c(C)c3)c(C#N)cnc2cc1OCCN(C)C ; CCOc1cc2ncnc(Nc3ccc(Oc4ccn5ncnc5c4)c(C)c3)c2cc1NC(=O)C#CCN1CCN(C)CC1 ; CC#CC(=O)Nc1c(OCCCN(C)C)cc2ncnc(Nc3ccc(Cc4ccn5ncnc5c4)c(C)c3)c2c1F ; CC#CC(=O)Nc1ccc2ncnc(Nc3cc(C)c(Oc4ccn5ncnc5c4)cc3F)c2c1F ; CC#CC(=O)Nc1ccc2ncnc(Nc3ccc(Oc4ccc5nccnc5c4)c(C)c3)c2c1] 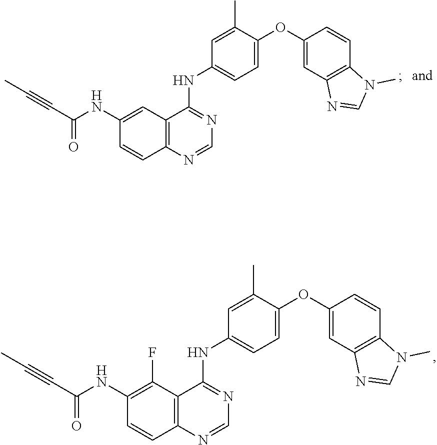
or a pharmaceutically acceptable salt thereof.

46. A pharmaceutical composition comprising the compound of claim 1, or a pharmaceutically acceptable salt thereof, and at least one pharmaceutically acceptable excipient.

47. A method of inhibiting kinase activity of a human receptor tyrosine kinase ErbB2 or a mutant form of human ErbB2 comprising contacting the ErbB2 or the mutant form with a therapeutically effective amount of the compound of claim 1, or a pharmaceutically acceptable salt thereof.

48-51. (canceled)

52. A method of treating a patient having a cancer, comprising administering to the patient a therapeutically effective amount of the compound of claim 1, or a pharmaceutically acceptable salt thereof.

53-54. (canceled)

55. The method of claim 52, wherein the cancer comprises cells or cell tissue having one or more mutations in Exon 20 of the ErbB2 that introduce amino acid deletions and/or insertions selected from the group consisting of A775_A776insYVMA, G778_P780insGSP, G776delinsVC, P780_Y781insGSP, M774delinsWLV, A775_G776insSVMA, A775_G776insI, G776delinsLC, G778_S779InsCPG, and V777_G778insGSP.

56. (canceled)

57. The method of claim 52, wherein the cancer comprises cells or cell tissue having one or more point mutations that introduce:
(a) an amino acid substitution selected from the group consisting of P122L, R217C, I263T, A293T, S305C, S310F/Y, H470Q, I655V, V659E, G660D, R678Q/C, L755R/S/P, I767M, D769H/N/Y, V777L/M, V842I, R868W, H878Y, E930K/D, E1021Q, F1030C, V1128I, and N1219S; or
(b) a frameshift at A1232.

58. The method of claim 52, wherein the cancer is lung, glioma, skin, head and neck, salivary gland, breast, esophageal, liver, stomach (gastric), uterine, cervical, biliary tract, pancreatic, colorectal, renal, bladder or prostate cancer.

59. (canceled)

60. The method of claim 52, wherein the patient has received at least one, at least two, or at least three prior therapies for the cancer.

61-62. (canceled)
